(19) United States
(12) Patent Application Publication
(10) Pub. No.: US 2024/0130232 A1
(43) Pub. Date: Apr. 18, 2024

(54) HETEROCYCLIC COMPOUND, ORGANIC LIGHT EMITTING DEVICE, AND COMPOSITION FOR ORGANIC MATERIAL LAYER OF ORGANIC LIGHT EMITTING DEVICE

(71) Applicant: LT MATERIALS CO., LTD., Yongin-City (KR)

(72) Inventors: Hyun-Joo LEE, Yongin-City (KR); Geon-Yu PARK, Yongin-City (KR); Young-Seok NO, Yongin-City (KR); Dong-Jun KIM, Yongin-City (KR); Dae-Hyuk CHOI, Yongin-City (KR)

(73) Assignee: LT MATERIALS CO., LTD., Yongin-City (KR)

(21) Appl. No.: 18/225,831

(22) Filed: Jul. 25, 2023

(30) Foreign Application Priority Data
Aug. 24, 2022 (KR) 10-2022-0106465

(51) Int. Cl. H10K 85/60; C07D 405/04; C07D 405/14; C07D 409/04; C07D 409/14; C09K 11/06

(52) U.S. Cl. CPC H10K 85/6574; C07D 405/04; C07D 405/14; C07D 409/04; C07D 409/14; C09K 11/06; H10K 85/615; H10K 85/654; H10K 85/6576; H10K 50/12

(57) ABSTRACT

The present specification relates to a heterocyclic compound, an organic light emitting device, and a composition for an organic material layer of an organic light emitting device.

### CROSS-REFERENCE TO RELATED APPLICATION

This application claims priority to and the benefit of Korean Patent Application No. 10-2022-0106465, filed on Aug. 24, 2022, the disclosure of which is incorporated herein by reference in its entirety.

### BACKGROUND

### 1. Field of the Invention

The present specification relates to a heterocyclic compound, an organic light emitting device, and a composition for an organic material layer of the organic light emitting device.

### 2. Discussion of Related Art

An electroluminescent device is a type of self-emissive display device, and it has a wide viewing angle, excellent contrast, and a fast response speed.

An organic light emitting device has a structure in which an organic thin film is disposed between two electrodes. When a voltage is applied to an organic light emitting device having such a structure, electrons and holes injected from the two electrodes are combined in an organic thin film to form pairs, and then the pairs disappear, thereby emitting light. The organic thin film may be formed of a single layer or multiple layers.

A material for the organic thin film may have a light-emitting function as needed. For example, as a material for the organic thin film, either a compound capable of constituting a light emissive layer by itself or a compound capable of serving as a host or dopant of a host-dopant-based light emissive layer may be used. Alternatively, as a material for the organic thin film, a compound capable of performing hole injection, hole transport, electron blocking, hole blocking, electron transport, and electron injection may be used.

To improve the performance, lifetime, or efficiency of an organic light emitting device, there is a consistent demand for the development of materials for an organic thin film.

### RELATED ART DOCUMENT

[Patent Document]
(Patent Document 1) U.S. Pat. No. 4,356,429

### SUMMARY OF THE INVENTION

The present specification is directed to providing a heterocyclic compound, an organic light emitting device, and a composition for an organic material layer of the organic light emitting device.

In one aspect, the present specification provides a heterocyclic compound of Formula 1 below.

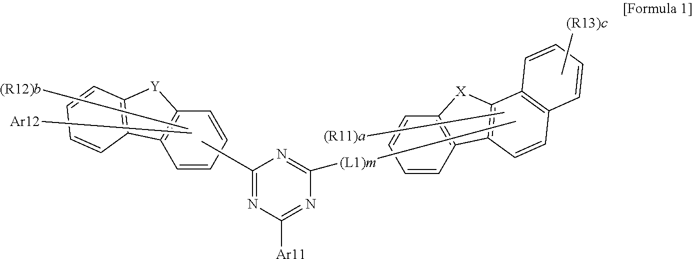

In Formula 1,
X and Y are the same or different, and are each independently O; S; or CRR′,
Ar11 and Ar12 are the same or different, and are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
L1 is a direct bond; a substituted or unsubstituted C6 to C60 arylene group; or a substituted or unsubstituted C2 to C60 heteroarylene group,
R11 to R13 are the same or different, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R and R′ are the same or different, and are each independently a substituted or unsubstituted C1 to C60 alkyl group; a substituted or unsubstituted C6 to C60 aryl group; or a substituted or unsubstituted C2 to C60 heteroaryl group,
m and c are the same or different, and are each independently an integer of 0 to 4,
a is an integer of 0 to 5,
b is an integer of 0 to 6,
when a to c, and m are 2 or more, the substituents in parentheses are the same or different,
the deuterium content in

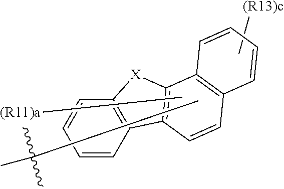

of Formula 1 is more than 0% and 100% or less,
the deuterium content of Formula 1 is less than 100%, and

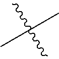

indicates the bonding position with Formula 1.

In another aspect, the present specification provides an organic light emitting device, which includes a first electrode; a second electrode disposed opposite to the first electrode; and one or more organic material layers disposed between the first electrode and the second electrode, wherein one or more of the organic material layers include the heterocyclic compound.

In still another aspect, a composition for an organic material layer of the organic light emitting device including a heterocyclic compound is provided.

### BRIEF DESCRIPTION OF THE DRAWINGS

The above and other objects, features and advantages of the present application will become more apparent to those of ordinary skill in the art by describing in detail exemplary embodiments thereof with reference to the accompanying drawings, in which:

FIGS. 1 to 3 are diagrams illustrating the stacked structures of an organic light emitting device according to one embodiment of the present specification, respectively.

### DETAILED DESCRIPTION OF EXEMPLARY EMBODIMENTS

Hereinafter, the present specification will be described in further detail.

In the present specification, when a part “includes” one component, it means that another component is further included, rather than excluding another component unless particularly stated otherwise.

In the present specification,

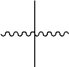

of the formula indicates a bonding position.

The term “substituted” refers to changing a hydrogen atom bonded to a carbon atom of a compound to another substituent, and the substituted position is not limited as long as it is a position that can be substituted with a substituent. When two or more positions are substituted, two or more substituents may be the same or different.

In the present specification, the “substituted” or “unsubstituted” means that one or more substituents selected from the group consisting of deuterium; a halogen group; a cyano group; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; a silyl group; a phosphine oxide group; and an amine group are substituted or unsubstituted, or two or more substituents selected from the above-listed substituents are substituted or unsubstituted with a linked substituent.

In the present specification, the “the case in which a substituent is not represented in a formula or compound structure” means a hydrogen atom is bonded to a carbon atom. However, deuterium (²H) may be an isotope of hydrogen, and some hydrogen atoms may be deuterium.

In one embodiment of the present application, the “case in which a substituent is not represented in a formula or compound structure” may mean that hydrogen or deuterium are present at all substitution positions as substituents. That is, deuterium is an isotope of hydrogen, and some hydrogen atoms may be deuterium as isotopes. Here, a deuterium content may be 0 to 100%.

In one embodiment of the present application, in the “case in which a substituent is not present in a formula or compound structure,” when a deuterium content is 0%, a hydrogen content is 100%, or all substituents are hydrogen, unless deuterium is explicitly excluded, hydrogen and deuterium may be used together in the compound.

In one embodiment of the present application, deuterium is one of the isotopes of hydrogen, which is an element having a deuteron consisting of one proton and one neutron as a nucleus. Deuterium is expressed as hydrogen-2, and its element symbol may also be written as D or ²H.

In one embodiment of the present application, isotopes meaning atoms with the same atomic number (Z) but different mass numbers (A) may be interpreted as elements with the same number of protons but different numbers of neutrons.

In one embodiment of the present application, the content T % of specific substituents may be defined as T2/T1×100=T % wherein the total number of substituents of a base compound is defined as T1, and the number of specific substituents among all substituents is defined as T2.

That is, in one example, in a phenyl group represented by

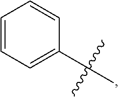

a deuterium content of 20% may be obtained when the total number of substituents of the phenyl group is 5 (T1 of the formula), and the number of deuterium atoms is 1 (T2 of the formula). That is, the phenyl groups having a deuterium content of 20% may be represented by the following structural formulas.

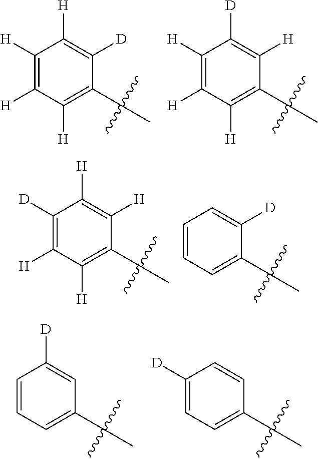

In addition, in one embodiment of the present application, a “phenyl group with a deuterium content of 0%” may mean a phenyl group that does not include a deuterium atom, that is, five hydrogen atoms.

In the present specification, the halogen may be fluorine, chlorine, bromine, or iodine.

In the present specification, the alkyl group may be a linear or branched chain having 1 to 60 carbon atoms, and may be further substituted by a different substituent. The alkyl group may have 1 to 60 carbon atoms, specifically, 1 to 40 carbon atoms, and more specifically, 1 to 20 carbon atoms. As a specific example, the alkyl group may be a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, a n-nonyl group, a 2,2-dimethylheptyl group, a 1-ethyl-propyl group, a 1,1-dimethyl-propyl group, an isohexyl group, a 2-methylpentyl group, a 4-methylhexyl group, or a 5-methylhexyl group, but the present application is not limited thereto.

In the present specification, the alkenyl group may be a linear or branched chain having 2 to 60 carbon atoms, and may be further substituted by a different substituent. The alkenyl group may have 2 to 60 carbon atoms, specifically 2 to 40 carbon atoms, and more specifically 2 to 20 carbon atoms. As a specific example, the alkenyl group may be a vinyl group, a 1-prophenyl group, an isoprophenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stylbenyl group, or a styrenyl group, but the present application is not limited thereto.

In the present specification, the alkynyl group may be a linear or branched chain having 2 to 60 carbon atoms, and may be further substituted by a different substituent. The alkynyl group may have 2 to 60 carbon atoms, specifically 2 to 40 carbon atoms, and more specifically 2 to 20 carbon atoms.

In the present specification, the alkoxy group may be a linear, branched or cyclic chain. The number of carbon atoms of the alkoxy group is not particularly limited, but is preferably 1 to 20. Specifically, the alkoxy group may be a methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, or p-methylbenzyloxy group, but the present application is not limited thereto.

In the present specification, the cycloalkyl group may include a monocyclic or polycyclic group having 3 to 60 carbon atoms, and may be further substituted by a different substituent. Here, a polycyclic group refers to a group in which a cycloalkyl group is directly bonded or condensed with another cyclic group. Here, the other cyclic group may be a cycloalkyl group, but may also be a different type of cyclic group, for example, a heterocycloalkyl group, an aryl group, and a heteroaryl group. The cycloalkyl group may have 3 to 60 carbon atoms, specifically, 3 to 40 carbon atoms, and more specifically 5 to 20 carbon atoms. Specifically, the cycloalkyl group may be a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, or a cyclooctyl group, but the present application is not limited thereto.

In the present specification, the heterocycloalkyl group may be a monocyclic or polycyclic group, which includes 0, S, Se, N or Si as a hetero atom and has 2 to 60 carbon atoms, and may be further substituted by a different substituent. Here, the polycyclic group refers to a group in which a cycloalkyl group is directly bonded or condensed with another cyclic group. Here, the other cyclic group may be a heterocycloalkyl group, but may also be a different type of cyclic group, for example, a cycloalkyl group, an aryl group, and a heteroaryl group. The heterocycloalkyl group may have 2 to 60 carbon atoms, specifically 2 to 40 carbon atoms, and further specifically, 3 to 20 carbon atoms.

In the present specification, the aryl group may include a monocyclic or polycyclic group having 6 to 60 carbon atoms, and may be further substituted by another substituent. Here, the polycyclic group refers to a group in which a cycloalkyl group is directly bonded or condensed with another cyclic group. Here, the other cyclic group may be an aryl group, but may also be a different type of cyclic group, for example, a cycloalkyl group, a heterocycloalkyl group, or a heteroaryl group. The aryl group includes a spiro group. The aryl group may have 6 to 60 carbon atoms, specifically 6 to 40 carbon atoms, and more specifically 6 to 25 carbon atoms. A specific example of aryl group may be a phenyl group, a non-phenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-TH-indenyl group, or a condensed cyclic group thereof, but the present application is not limited thereto.

In the present specification, the terphenyl group may be selected from the following structures.

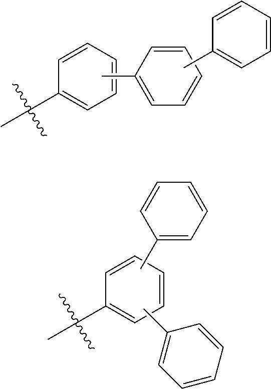

In the present specification, the fluorenyl group may be substituted, and adjacent substituents may be bonded with each other, thereby forming a ring.

When the fluorenyl group is substituted,

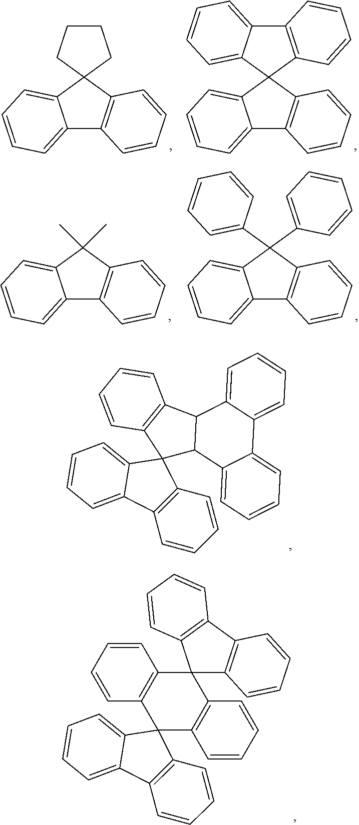

etc. may be formed, but the present application is not limited thereto.

In the present specification, the heteroaryl group may be a monocyclic or polycyclic group, which includes S, O, Se, N, or Si as a hetero atom and has 2 to 60 carbon atoms, and may be further substituted by a different substituent. Here, the polycyclic group refers to a group in which a cycloalkyl group is directly bonded or condensed with another cyclic group. Here, the other cyclic group may be a heteroaryl group, or may be a different type of cyclic group, such as a cycloalkyl group, a heterocycloalkyl group, or an aryl group. The heteroaryl group may have 2 to 60 carbon atoms, specifically 2 to 40 carbon atoms, and more specifically 3 to 25 carbon atoms. A specific example of heteroaryl group may be a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiazolyl group, dithiazolyl group, a tetrazolyl group, a pyranyl group, a thiophyranyl group, a diazinyl group, an oxazinyl group, a thiazinyl group, dioxynyl group, a triazinyl group, a tetrazinyl group, a quinolyl group, an isoquinolyl group, a quinazolinyl group an isoquinazolinyl group, a quinoxalinyl group, a naphthyridyl group, an acridinyl group, a phenanthridynyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilol group, a spirobi(dibenzosilole), dihydrophenazinyl group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 2,3-dihydrobenzo[b]thiophene, a 2,3-dihydrobenzofuran, a 5,10-dihydrodibenzo[b,e][1,4]azasilynyl, a pyrazolo[1,5-c]quinazolinyl group, a pyrido[1,2-b]indazolyl group, a pyrido[1,2-a]imidazo[1,2-e]indolinyl group, or a 5,11-dihydroindeno[1,2-b]carbazolyl group, but the present application is not limited thereto.

In the present specification, a silyl group is a substituent including Si, in which the Si atom is directly connected as a radical. The silyl group is represented by —Si(R101)(R102)(R103), in which R101 to R103 are the same or different and are each independently a substituent consisting of at least one of hydrogen; deuterium; a halogen group; an alkyl group; an alkenyl group; an alkoxy group; a cycloalkyl group; a heterocycloalkyl group; an aryl group; and a heteroaryl group. A specific example of the silyl group may be

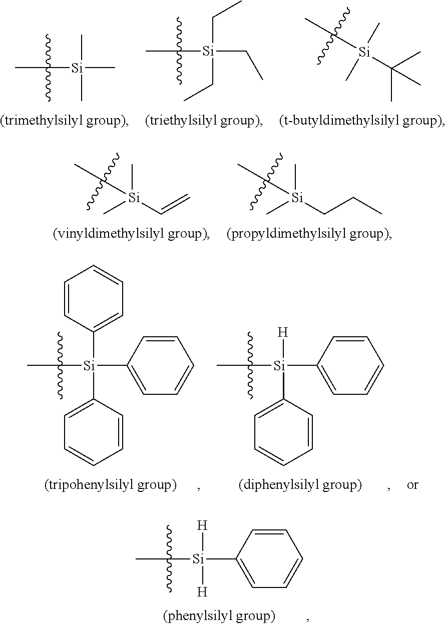

but the present application is not limited thereto.

In the present specification, the phosphine oxide group may be represented by —P(═O)(R104)(R105), in which R104 and R105 are the same or different and are each independently a substituent consisting of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described examples may be applied for the alkyl group and the aryl group. For example, the phosphine oxide group may be a dimethylphosphine oxide group, a diphenylphosphine oxide group, or a dinaphthylphosphine oxide group, but the present application is not limited thereto.

In the present specification, the amine group may be represented by —N(R106)(R107), in which R106 and R107 are the same or different and are each independently a substituent consisting of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diaryamine group; a diheteroarylamine group; an alkylarylamine group; an alkylheteroarylamine group; and an arylheteroarylamine group, and preferably has 1 to 30 carbon atoms, but not limited thereto. A specific example of the amine group may be a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, or a biphenyltriphenylenylamine group, but the present application is not limited thereto.

In the present specification, the above-described examples of aryl groups may be applied to the arylene group, except for a divalent arylene group.

In the present specification, the above-described examples of heteroaryl groups may be applied to the heteroarylene group, except for a divalent heteroarylene group.

In one embodiment of the present specification, a heterocyclic compound of Formula 1 is provided.

In one embodiment of the present application, X and Y are the same or different, and are each independently O; S; or CRR′.

In one embodiment of the present application, X may be O.

In one embodiment of the present application, X may be S

In one embodiment of the present application, Y may be O.

In one embodiment of the present application, Y may be S.

In one embodiment of the present application, X may be O, and Y may be O.

In one embodiment of the present application, X may be O, and Y may be S.

In one embodiment of the present application, X may be S, and Y may be S.

In one embodiment of the present application, X may be S, and Y may be O.

In one embodiment of the present application, Ar11 may be a substituted or unsubstituted C1 to C60 alkyl group; a substituted or unsubstituted C3 to C60 cycloalkyl group; a substituted or unsubstituted C2 to C60 heterocycloalkyl groups; a substituted or unsubstituted C6 to C60 aryl group; or a substituted or unsubstituted C2 to C60 heteroaryl group.

In another embodiment, Ar11 is a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another embodiment, Ar1 is a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In yet another embodiment, Ar11 is a C1 to C20 alkyl group; a C3 to C20 cycloalkyl group; a C2 to C20 heterocycloalkyl group; a C6 to C20 aryl group; or a C2 to C20 heteroaryl group.

In yet another embodiment, Ar11 is a C6 to C20 aryl group.

In yet another embodiment, Ar11 may be a substituted or unsubstituted phenyl group; a substituted or unsubstituted biphenyl group; or a substituted or unsubstituted terphenyl group.

In yet another embodiment, Ar11 may be a phenyl group; a biphenyl group; or a terphenyl group.

In one embodiment of the present application, Ar12 is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another embodiment, Ar12 is a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another embodiment, Ar12 is a substituted or unsubstituted C6 to C40 aryl group.

In yet another embodiment, Ar12 is a C6 to C40 aryl group.

In yet another embodiment, Ar12 is a monocyclic C6 to C10 aryl group; or a polycyclic C10 to C40 aryl group.

In yet another embodiment, Ar12 is a monocyclic C6 to C10 aryl group.

In yet another embodiment, Ar12 is a substituted or unsubstituted phenyl group.

In yet another embodiment, Ar12 is a phenyl group.

In one embodiment of the present application, R11 to R13 are the same or different, and each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another embodiment, R11 to R13 are the same or different, and each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In still another embodiment, R11 to R13 are the same or different, and each independently hydrogen; deuterium; a substituted or unsubstituted C1 to C40 alkyl group; a substituted or unsubstituted C6 to C40 aryl group; or a substituted or unsubstituted C2 to C40 heteroaryl group.

In yet another embodiment, R11 to R13 are the same or different, and each independently hydrogen; deuterium; a C1 to C40 alkyl group; a C6 to C40 aryl group; or a C2 to C40 heteroaryl group.

In yet another embodiment, R11 to R13 are the same or different, and each independently hydrogen or deuterium.

In one embodiment of the present application, R11 may be deuterium, and a may be an integer of 5.

In one embodiment of the present application, R13 may be deuterium, and c may be an integer of 4.

In one embodiment of the present application, R12 may be hydrogen, and b may be an integer of 6.

In one embodiment of the present application, L1 is a direct bond; a substituted or unsubstituted C6 to C60 arylene group; or a substituted or unsubstituted C2 to C60 heteroarylene group.

In another embodiment, L1 is a direct bond; a substituted or unsubstituted C6 to C40 arylene group; or a substituted or unsubstituted C2 to C40 heteroarylene group.

In still another embodiment, L1 is a direct bond; or a substituted or unsubstituted C6 to C40 arylene group.

In yet another embodiment, L1 is a direct bond; or a C6 to C40 arylene group.

In yet another embodiment, L1 is a direct bond; or a C6 to C20 arylene group.

In yet another embodiment, L1 is a direct bond; a substituted or unsubstituted phenylene group; or a substituted or unsubstituted biphenylene group.

In yet another embodiment, L1 is a direct bond; a phenylene group; or a biphenylene group.

In one embodiment of the present application, Formula 1 provides a heterocyclic compound represented by Formula 3 or 4 below.

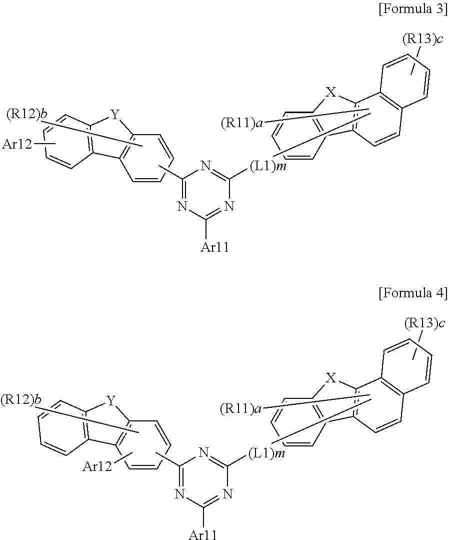

In Formulas 3 and 4,
the definition of each substituent is the same as in Formula 1.

In one embodiment of the present application, Formula 3 provides a heterocyclic compound represented by any one of Formulas 3-1 to 3-4 below.

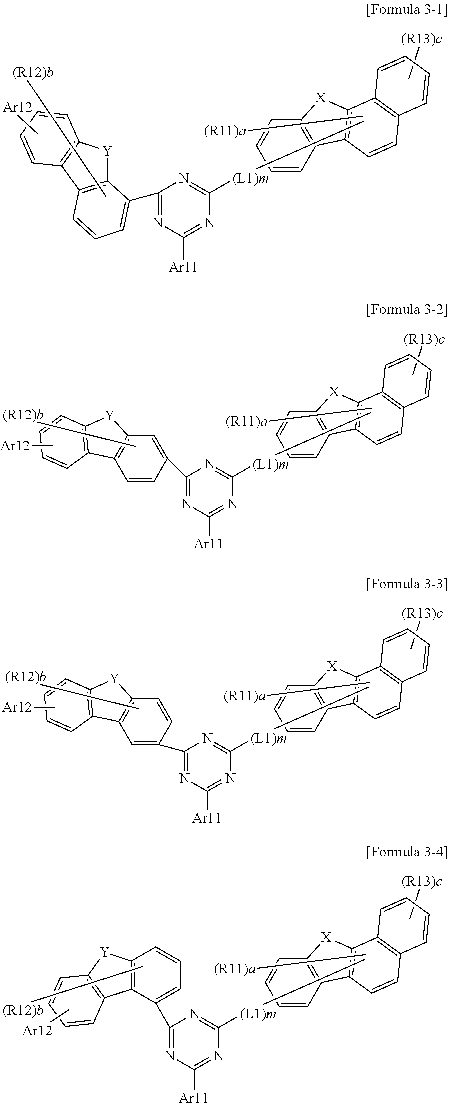

In Formulas 3-1 to 3-4,
the definition of each substituent is the same as in Formula 3.

In one embodiment of the present application, Formula 4 provides a heterocyclic compound represented by Formulas 4-1 to 4-4 below.

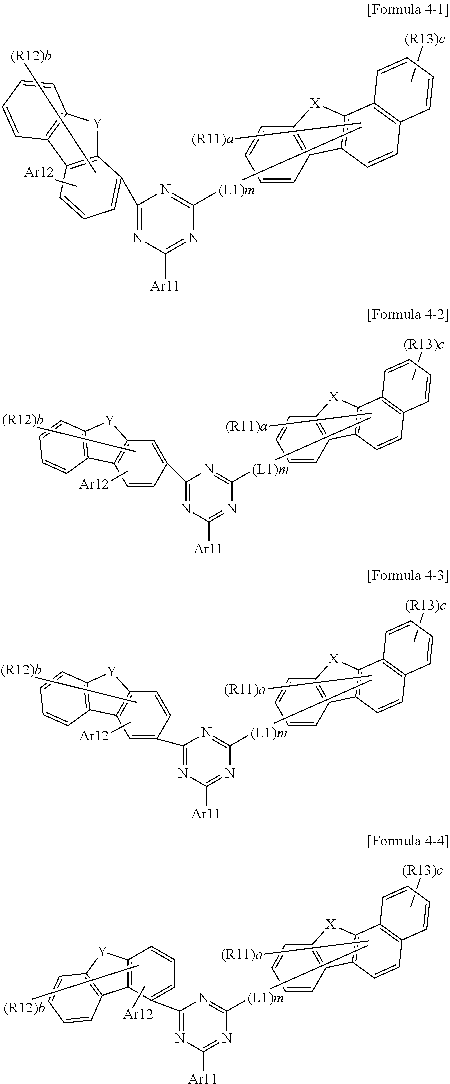

In Formulas 4-1 to 4-4,
the definition of each substituent is the same as in Formula 4.

In one embodiment of the present application,

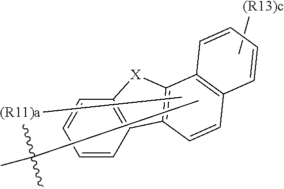

of Formula 1 provides a heterocyclic compound represented by any one of Formulas 1-1 to 1-6 below.

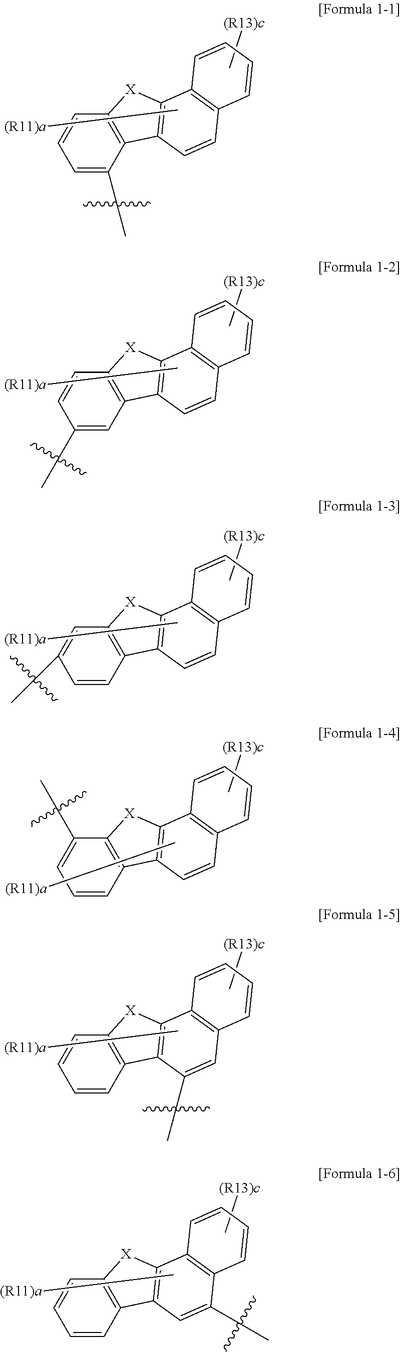

In Formulas 1-1 to 1-6,
the definition of each substituent is the same as in Formula 1.

In one embodiment of the present application, Formula 1 may include the structures of Formulas A to C below.

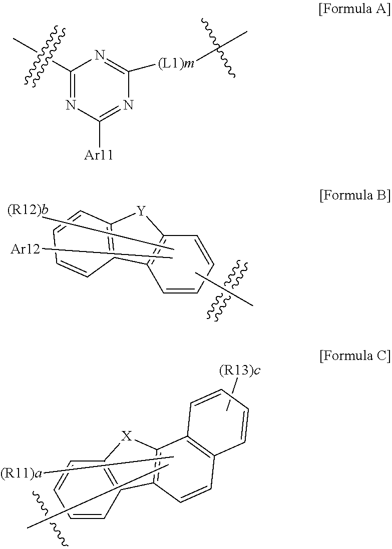

In Formulas A to C,

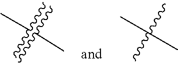

in the structures indicate the positions where the corresponding structure are bonded,
the definition of each substituent is the same as in Formula 1.

In one embodiment of the present application, the deuterium content in Formula A may be 0% to 100%.

In another embodiment, the deuterium content in Formula A may be 0% to 90%.

In still another embodiment, the deuterium content in Formula A may be 0% to 80%, or 0% to 50%, and preferably 0%.

In one embodiment of the present application, the deuterium content in Formula B may be 0% to 100%.

In another embodiment, the deuterium content in Formula B may be 0% to 90%.

In still another embodiment, the deuterium content in Formula B may be 0% to 80%, or 0% to 50%, and preferably, 0%.

In one embodiment of the present application, the deuterium content in Formula C may be 50% to 100%.

In yet another embodiment, the deuterium content in Formula C may be 60% to 100%, 70% to 100%, 80% to 100%, or 90% to 100%, and preferably 100%.

In one embodiment of the present application, Formula 1 may include the structures of Formulas A to C. Here, the deuterium content in Formula C may be 50% or more and 100% or less, and the contents of deuterium in Formulas A and B may be 0% or more and 10% or less.

In one embodiment of the present application, a heterocyclic compound in which the deuterium content of

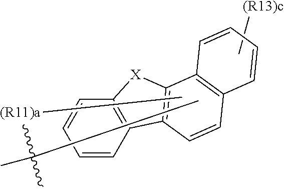

in Formula 1 is more than 50% and 100% or less is provided.

In one embodiment of the present application,

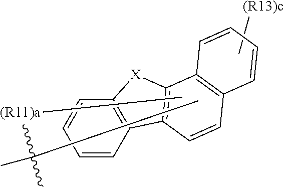

of Formula 1 provides a heterocyclic compound, which is any one of Formulas A-1 to A-4 below.

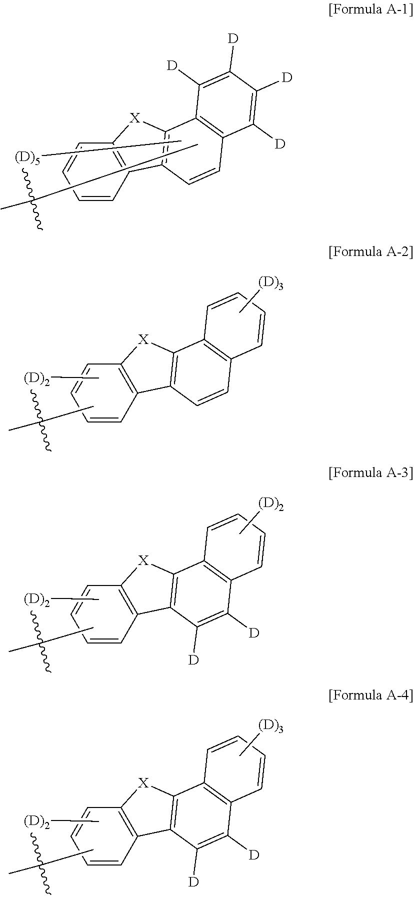

In Formulas A-1 to A-4,

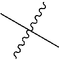

indicates the bonding position with Formula 1,
X is the same as the definition in Formula 1.

In one embodiment of the present application, in (D)ₓ of Formulas A-1 to A-4, x may represent the number of deuterium atoms. For example, (D)₅ in Formula A-1 may represent 5 deuterium atoms, and an example of Formulas A-1 to A-4 in which (D)ₓ is (D)₅ may has the following structure.

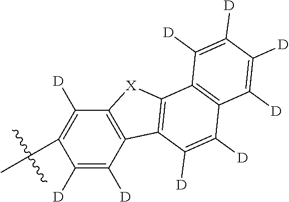

In one embodiment of the present application, all of L1, Ar11, Ar12, and R11 to R13 may include undeuterated H.

In one embodiment of the present application, at least one of L1, Ar11, Ar12, and R11 to R13 may include D, and at least one of L1, Ar11, Ar12, and R11 to R13 may include at least one undeuterated H.

In one embodiment of the present application, all of L1, Ar11, Ar12, and R11 to R13 may include D.

In one embodiment of the present specification, while the compound not including deuterium and the compound including deuterium have almost similar photochemical characteristics, when deposited as a thin film, the material including deuterium has a tendency to be packed with a narrower intermolecular distance.

Therefore, when an electron only device (EOD) and a hole only device (HOD) are manufactured and the current density according to voltage for each device is checked, it can be seen that the compound of Formula 1 according to the present application including deuterium exhibits a much more balanced charge transport property than the compound not including deuterium.

In addition, observing the thin film surface with an atomic force microscope (AFM), it can be confirmed that a thin film made of the compound including deuterium is deposited with a more uniform surface without an aggregated part.

In one embodiment of the present specification, Formula 1 may be represented by any one of the following compounds.

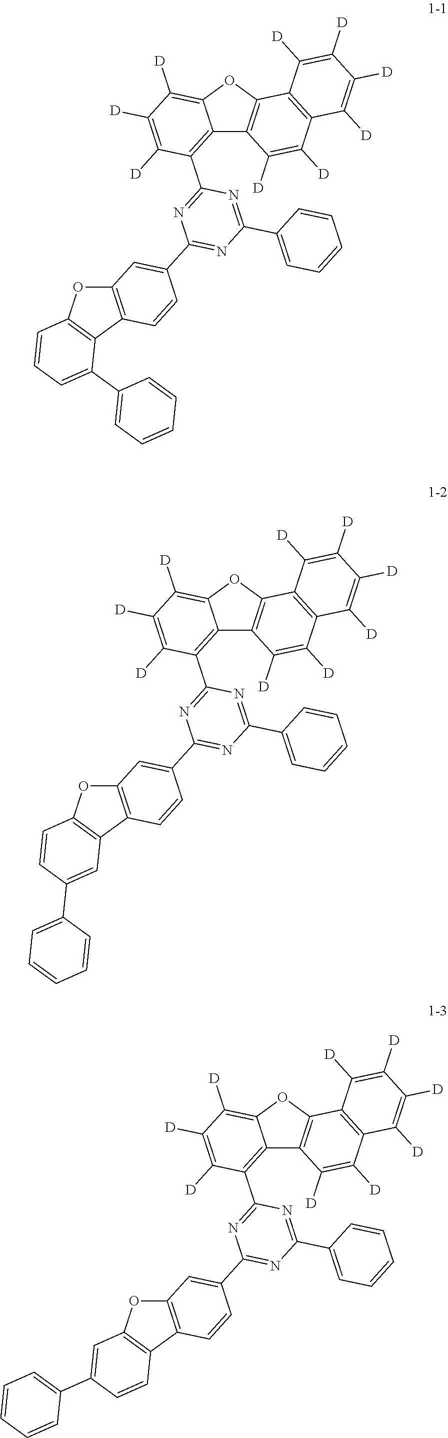 [SMILES: [2H]c1c([2H])c(-c2nc(-c3ccccc3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3ccc(-c5ccccc5)cc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3cc(-c5ccccc5)ccc34)n2)c2c(oc3c4c([2H])c([2H])c([2H])c([2H])c4c([2H])c([2H])c32)c1[2H]]

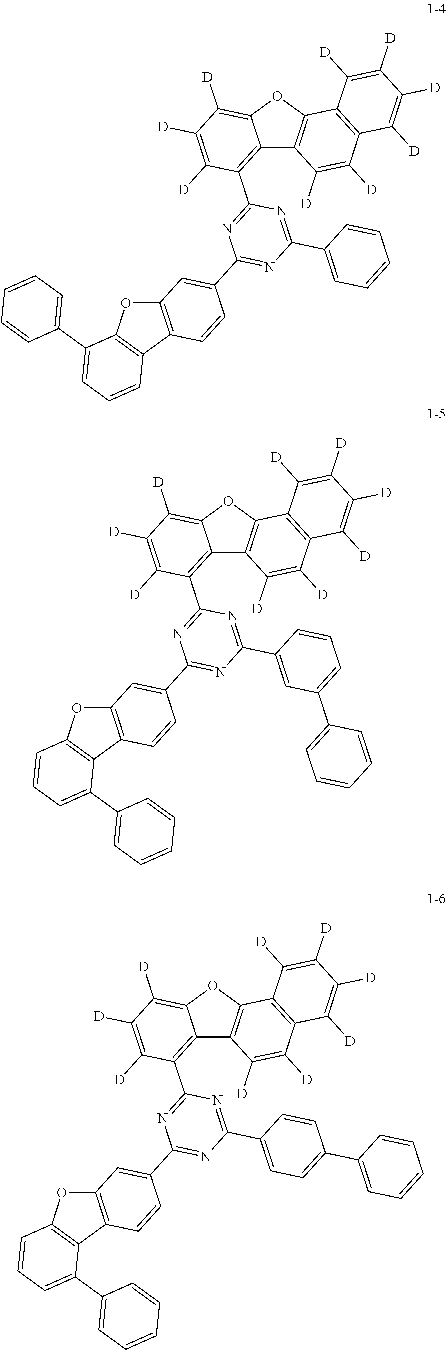 [SMILES: [2H]c1c([2H])c(-c2nc(-c3ccccc3)nc(-c3ccc4c(c3)oc3c(-c5ccccc5)cccc34)n2)c2c(oc3c4c([2H])c([2H])c([2H])c([2H])c4c([2H])c([2H])c32)c1[2H] ; [2H]c1c([2H])c(-c2nc(-c3cccc(-c4ccccc4)c3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccc(-c4ccccc4)cc3)nc(-c3ccc4c(c3)oc3cccc(-c5ccccc5)c34)n2)c2c(oc3c4c([2H])c([2H])c([2H])c([2H])c4c([2H])c([2H])c32)c1[2H]]

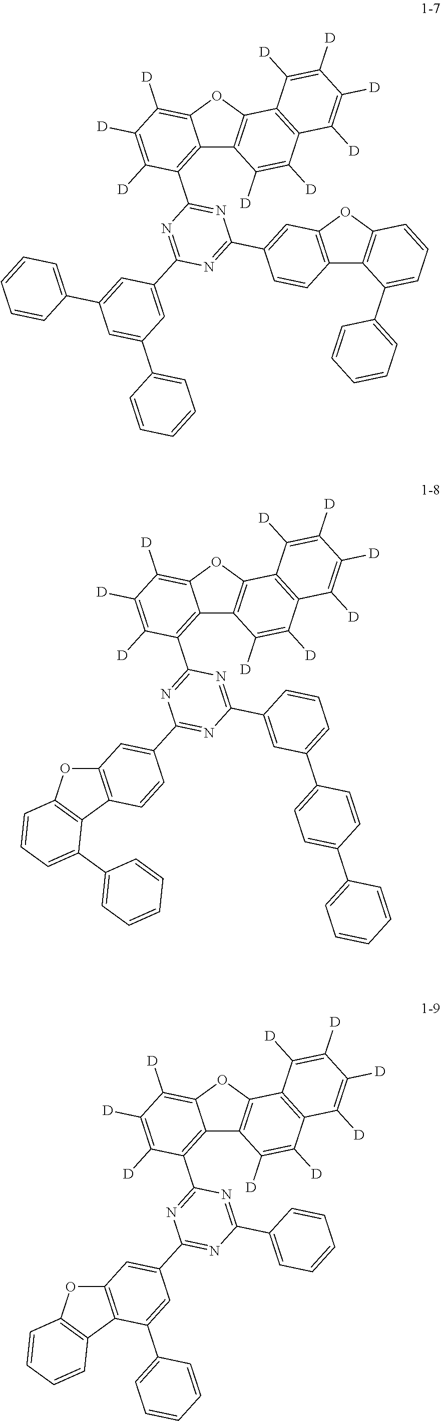 [SMILES: [2H]c1c([2H])c(-c2nc(-c3cc(-c4ccccc4)cc(-c4ccccc4)c3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3cccc(-c4ccc(-c5ccccc5)cc4)c3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3cc(-c4ccccc4)c4c(c3)oc3ccccc34)n2)c2c(oc3c4c([2H])c([2H])c([2H])c([2H])c4c([2H])c([2H])c32)c1[2H]]

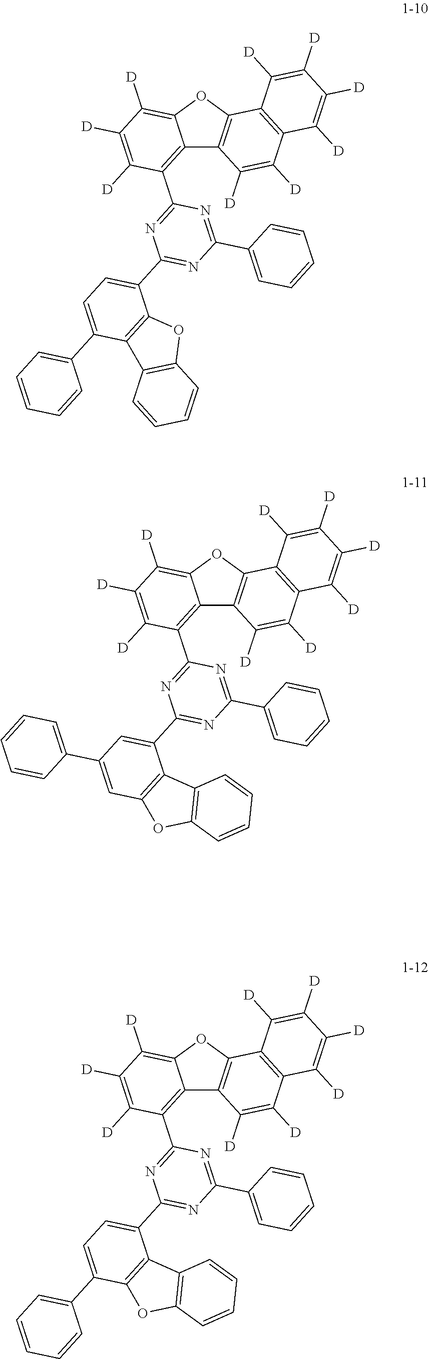 [SMILES: [2H]c1c([2H])c(-c2nc(-c3ccccc3)nc(-c3ccc(-c4ccccc4)c4c3oc3ccccc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3cc(-c4ccccc4)cc4oc5ccccc5c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc(-c4ccccc4)c4oc5ccccc5c34)n2)c2c(oc3c4c([2H])c([2H])c([2H])c([2H])c4c([2H])c([2H])c32)c1[2H]]

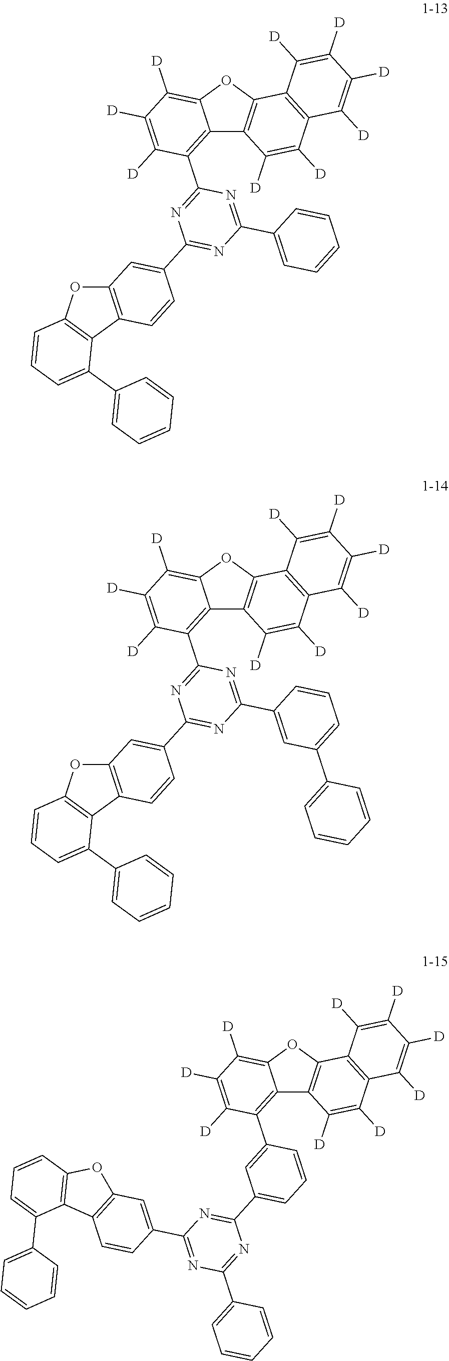 [SMILES: [2H]c1c([2H])c(-c2nc(-c3ccccc3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3cccc(-c4ccccc4)c3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2cccc(-c3nc(-c4ccccc4)nc(-c4ccc5c(c4)oc4cccc(-c6ccccc6)c45)n3)c2)c2c(oc3c4c([2H])c([2H])c([2H])c([2H])c4c([2H])c([2H])c32)c1[2H]]

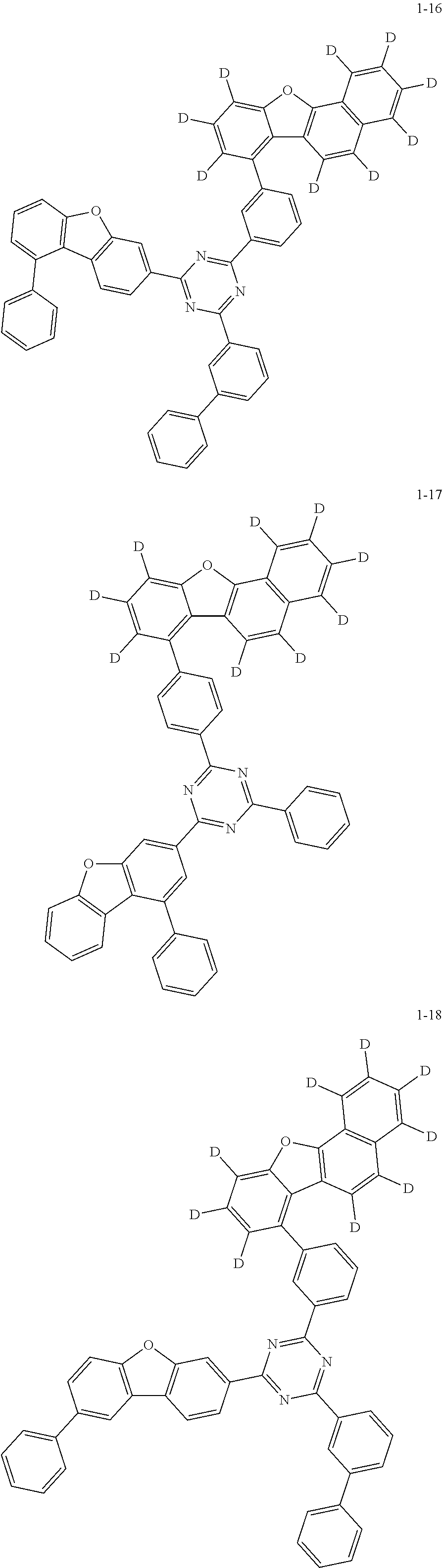 [SMILES: [2H]c1c([2H])c(-c2cccc(-c3nc(-c4cccc(-c5ccccc5)c4)nc(-c4ccc5c(c4)oc4cccc(-c6ccccc6)c45)n3)c2)c2c(oc3c4c([2H])c([2H])c([2H])c([2H])c4c([2H])c([2H])c32)c1[2H] ; [2H]c1c([2H])c(-c2ccc(-c3nc(-c4ccccc4)nc(-c4cc(-c5ccccc5)c5c(c4)oc4ccccc45)n3)cc2)c2c(oc3c4c([2H])c([2H])c([2H])c([2H])c4c([2H])c([2H])c32)c1[2H] ; [2H]c1c([2H])c(-c2cccc(-c3nc(-c4cccc(-c5ccccc5)c4)nc(-c4ccc5c(c4)oc4ccc(-c6ccccc6)cc45)n3)c2)c2c(oc3c4c([2H])c([2H])c([2H])c([2H])c4c([2H])c([2H])c32)c1[2H]]

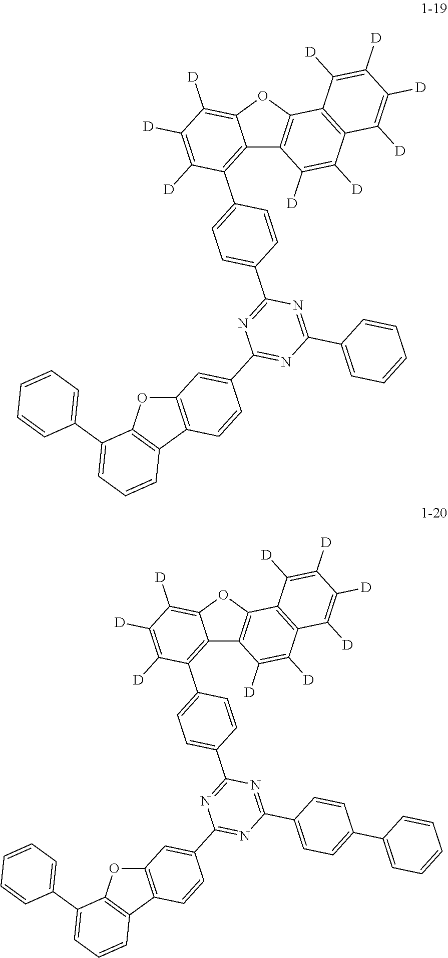 [SMILES: [2H]c1c([2H])c(-c2ccc(-c3nc(-c4ccccc4)nc(-c4ccc5c(c4)oc4c(-c6ccccc6)cccc45)n3)cc2)c2c(oc3c4c([2H])c([2H])c([2H])c([2H])c4c([2H])c([2H])c32)c1[2H] ; [2H]c1c([2H])c(-c2ccc(-c3nc(-c4ccc(-c5ccccc5)cc4)nc(-c4ccc5c(c4)oc4c(-c6ccccc6)cccc45)n3)cc2)c2c(oc3c4c([2H])c([2H])c([2H])c([2H])c4c([2H])c([2H])c32)c1[2H]]

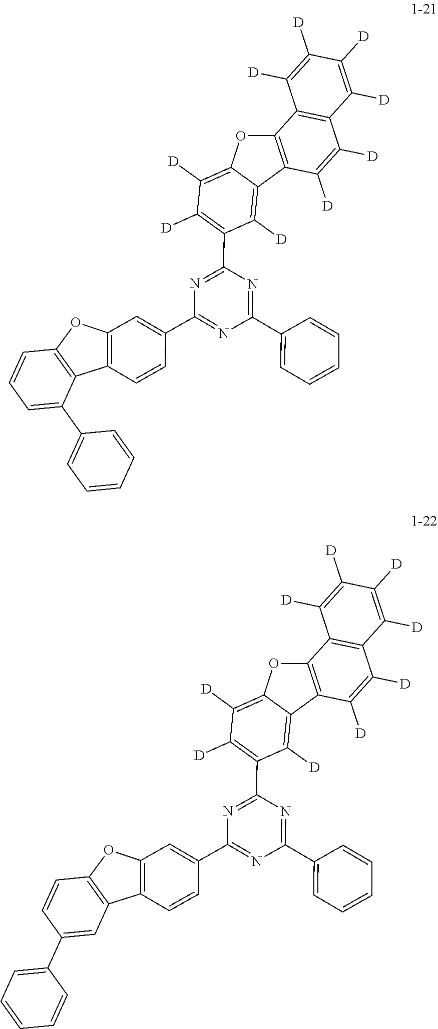 [SMILES: [2H]c1c(-c2nc(-c3ccccc3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)oc3ccc(-c5ccccc5)cc34)n2)c([2H])c2c(oc3c4c([2H])c([2H])c([2H])c([2H])c4c([2H])c([2H])c32)c1[2H]]

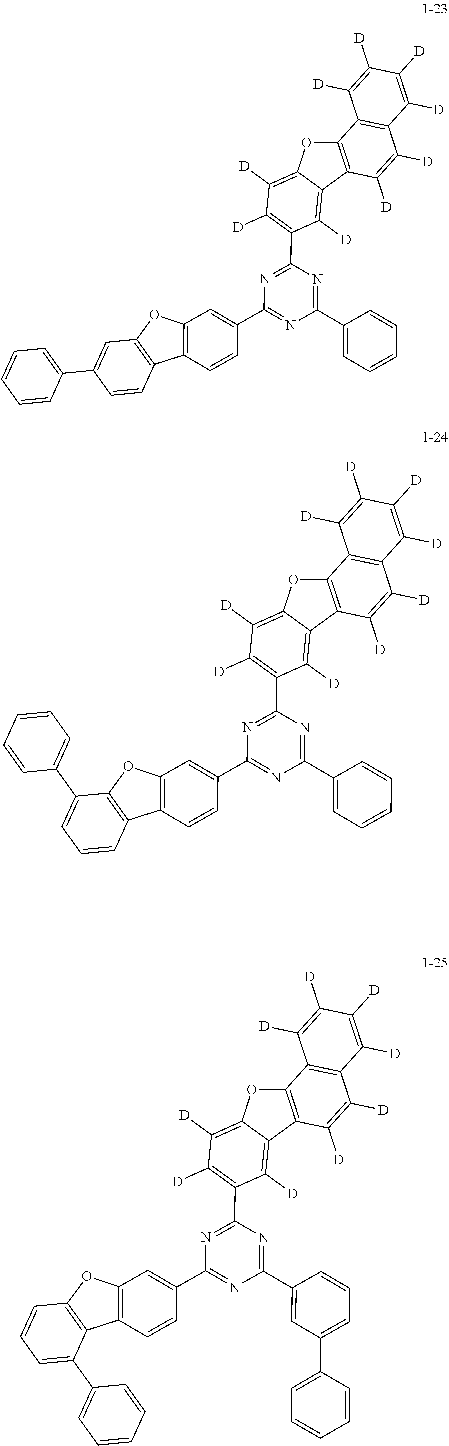 [SMILES: [2H]c1c(-c2nc(-c3ccccc3)nc(-c3ccc4c(c3)oc3cc(-c5ccccc5)ccc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)oc3c(-c5ccccc5)cccc34)n2)c([2H])c2c(oc3c4c([2H])c([2H])c([2H])c([2H])c4c([2H])c([2H])c32)c1[2H] ; [2H]c1c(-c2nc(-c3cccc(-c4ccccc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]]

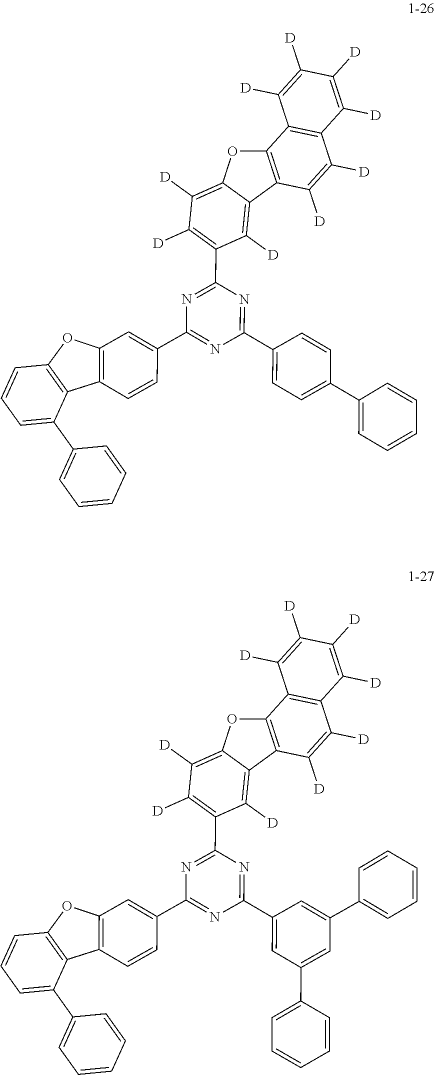 [SMILES: [2H]c1c(-c2nc(-c3ccc(-c4ccccc4)cc3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3cc(-c4ccccc4)cc(-c4ccccc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]]

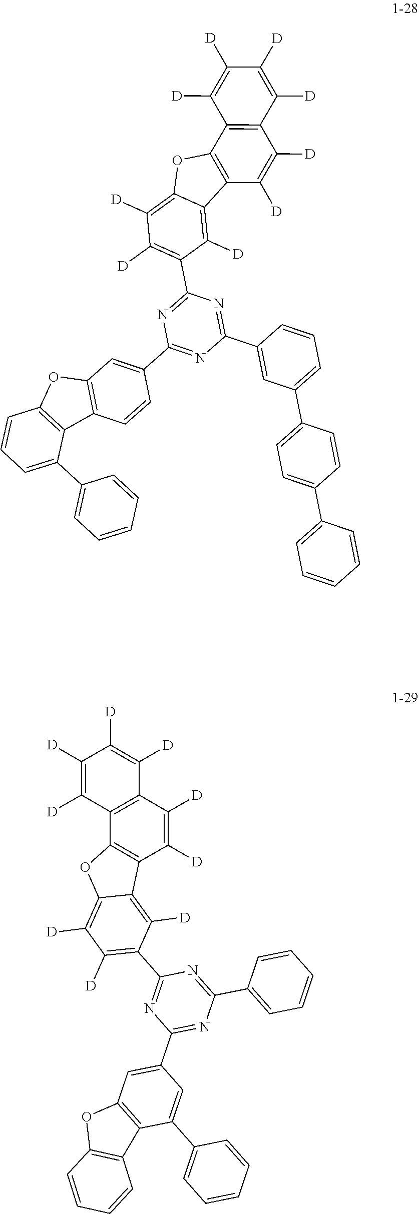 [SMILES: [2H]c1c(-c2nc(-c3cccc(-c4ccc(-c5ccccc5)cc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3cc(-c4ccccc4)c4c(c3)oc3ccccc34)n2)c([2H])c2c(oc3c4c([2H])c([2H])c([2H])c([2H])c4c([2H])c([2H])c32)c1[2H]]

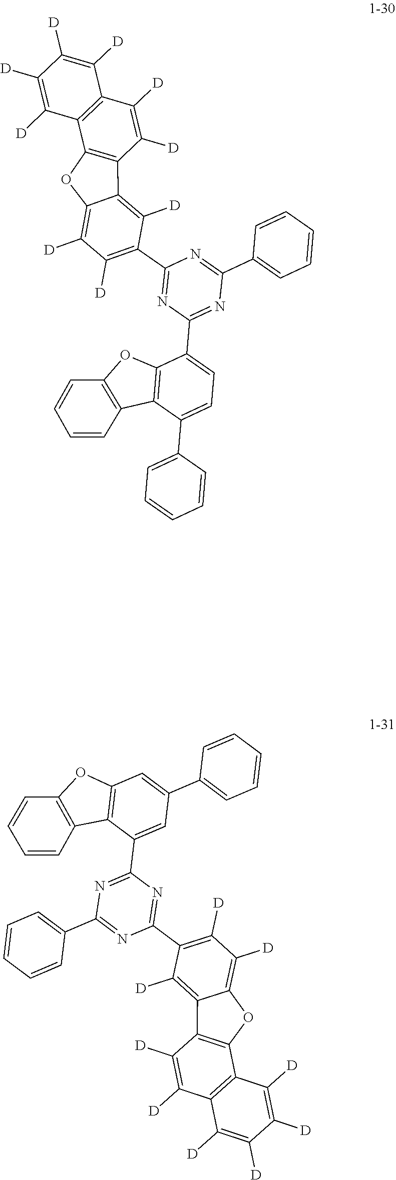 [SMILES: [2H]c1c(-c2nc(-c3ccccc3)nc(-c3ccc(-c4ccccc4)c4c3oc3ccccc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3cc(-c4ccccc4)cc4oc5ccccc5c34)n2)c([2H])c2c(oc3c4c([2H])c([2H])c([2H])c([2H])c4c([2H])c([2H])c32)c1[2H]]

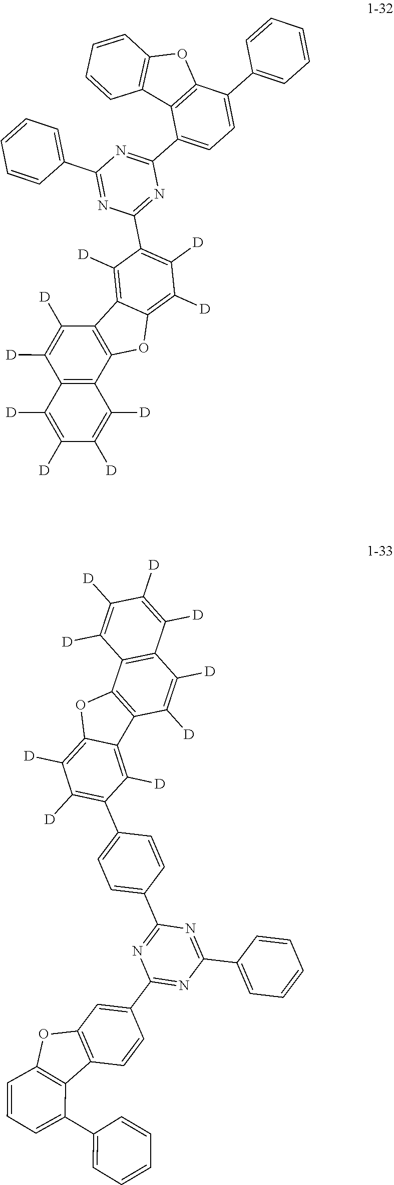 [SMILES: [2H]c1c(-c2nc(-c3ccccc3)nc(-c3ccc(-c4ccccc4)c4oc5ccccc5c34)n2)c([2H])c2c(oc3c4c([2H])c([2H])c([2H])c([2H])c4c([2H])c([2H])c32)c1[2H] ; [2H]c1c(-c2ccc(-c3nc(-c4ccccc4)nc(-c4ccc5c(c4)oc4cccc(-c6ccccc6)c45)n3)cc2)c([2H])c2c(oc3c4c([2H])c([2H])c([2H])c([2H])c4c([2H])c([2H])c32)c1[2H]]

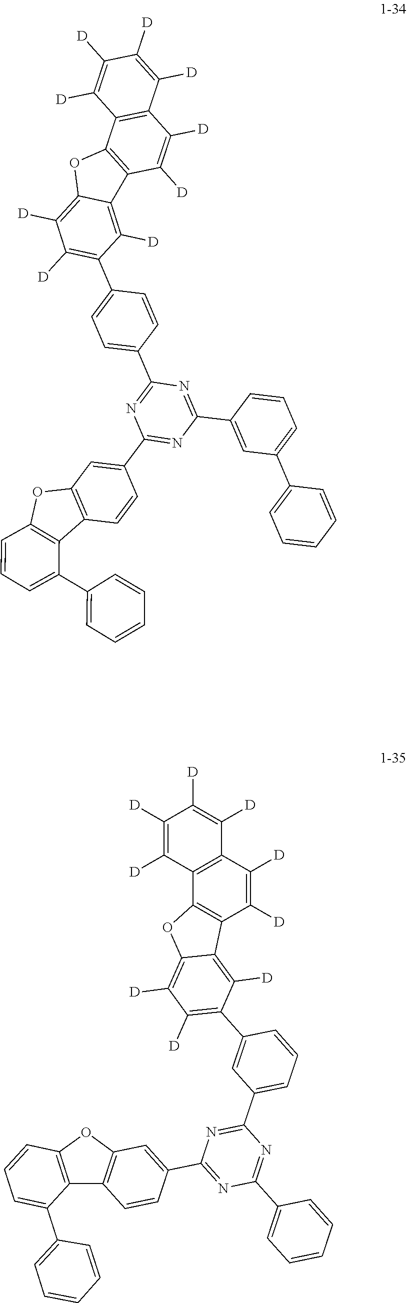 [SMILES: [2H]c1c(-c2ccc(-c3nc(-c4cccc(-c5ccccc5)c4)nc(-c4ccc5c(c4)oc4cccc(-c6ccccc6)c45)n3)cc2)c([2H])c2c(oc3c4c([2H])c([2H])c([2H])c([2H])c4c([2H])c([2H])c32)c1[2H] ; [2H]c1c(-c2cccc(-c3nc(-c4ccccc4)nc(-c4ccc5c(c4)oc4cccc(-c6ccccc6)c45)n3)c2)c([2H])c2c(oc3c4c([2H])c([2H])c([2H])c([2H])c4c([2H])c([2H])c32)c1[2H]]

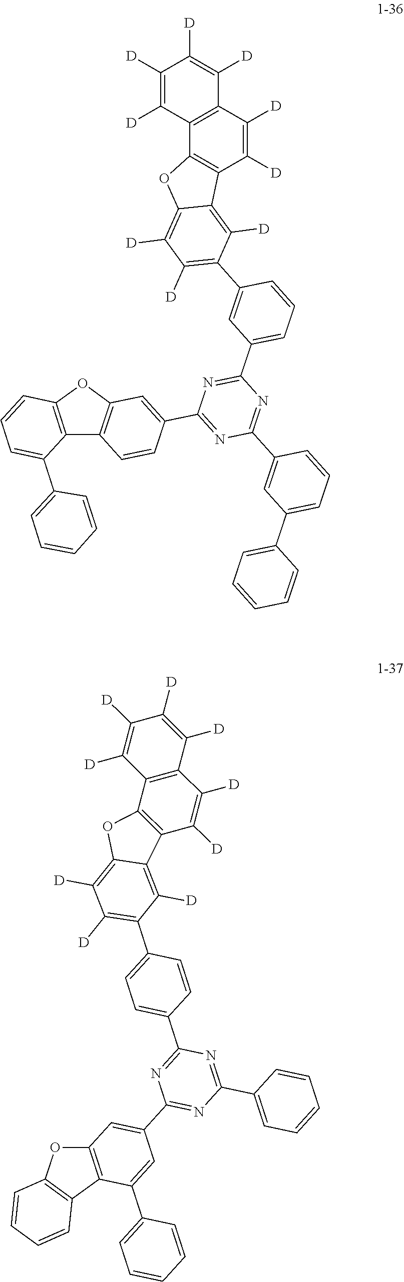 [SMILES: [2H]c1c(-c2cccc(-c3nc(-c4cccc(-c5ccccc5)c4)nc(-c4ccc5c(c4)oc4cccc(-c6ccccc6)c45)n3)c2)c([2H])c2c(oc3c4c([2H])c([2H])c([2H])c([2H])c4c([2H])c([2H])c32)c1[2H] ; [2H]c1c(-c2ccc(-c3nc(-c4ccccc4)nc(-c4cc(-c5ccccc5)c5c(c4)oc4ccccc45)n3)cc2)c([2H])c2c(oc3c4c([2H])c([2H])c([2H])c([2H])c4c([2H])c([2H])c32)c1[2H]]

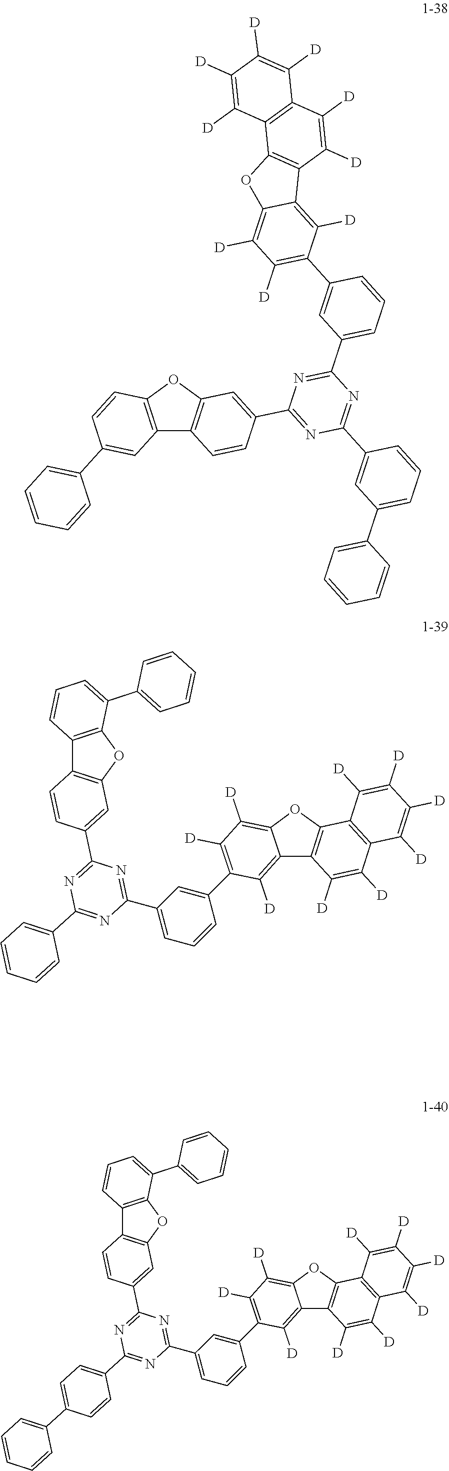 [SMILES: [2H]c1c(-c2cccc(-c3nc(-c4cccc(-c5ccccc5)c4)nc(-c4ccc5c(c4)oc4ccc(-c6ccccc6)cc45)n3)c2)c([2H])c2c(oc3c4c([2H])c([2H])c([2H])c([2H])c4c([2H])c([2H])c32)c1[2H] ; [2H]c1c(-c2cccc(-c3nc(-c4ccccc4)nc(-c4ccc5c(c4)oc4c(-c6ccccc6)cccc45)n3)c2)c([2H])c2c(oc3c4c([2H])c([2H])c([2H])c([2H])c4c([2H])c([2H])c32)c1[2H] ; [2H]c1c(-c2cccc(-c3nc(-c4ccc(-c5ccccc5)cc4)nc(-c4ccc5c(c4)oc4c(-c6ccccc6)cccc45)n3)c2)c([2H])c2c(oc3c4c([2H])c([2H])c([2H])c([2H])c4c([2H])c([2H])c32)c1[2H]]

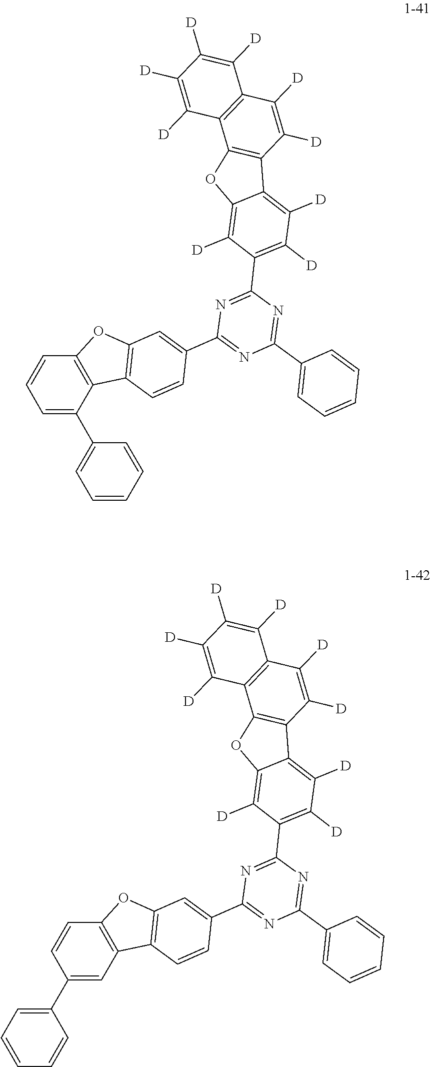 [SMILES: [2H]c1c([2H])c([2H])c2c(c1[2H])c([2H])c([2H])c1c2oc2c([2H])c(-c3nc(-c4ccccc4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)oc4ccc(-c6ccccc6)cc45)n3)c([2H])c([2H])c21]

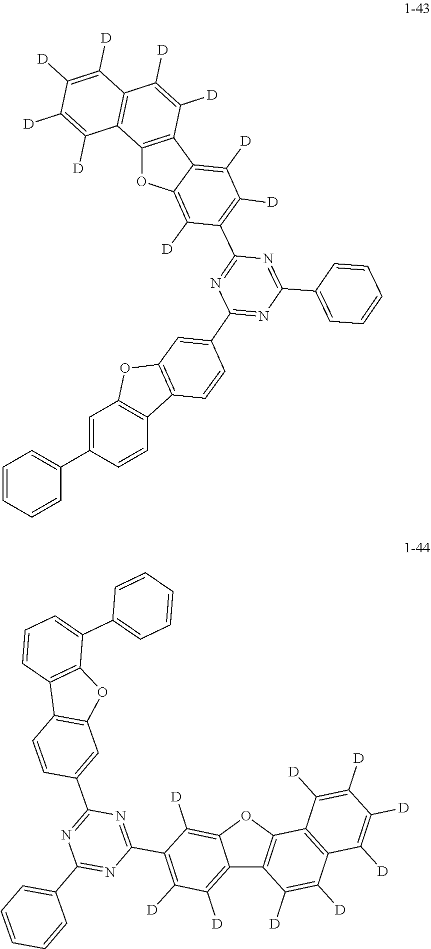 [SMILES: [2H]c1c([2H])c([2H])c2c(c1[2H])c([2H])c([2H])c1c2oc2c([2H])c(-c3nc(-c4ccccc4)nc(-c4ccc5c(c4)oc4cc(-c6ccccc6)ccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)oc4c(-c6ccccc6)cccc45)n3)c([2H])c([2H])c21]

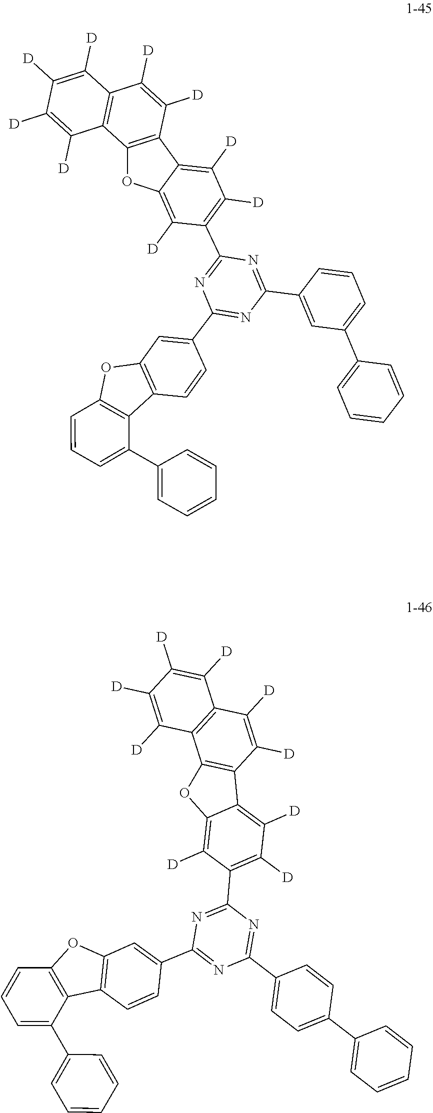 [SMILES: [2H]c1c([2H])c([2H])c2c(c1[2H])c([2H])c([2H])c1c2oc2c([2H])c(-c3nc(-c4cccc(-c5ccccc5)c4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4ccc(-c5ccccc5)cc4)nc(-c4ccc5c(c4)oc4cccc(-c6ccccc6)c45)n3)c([2H])c([2H])c21]

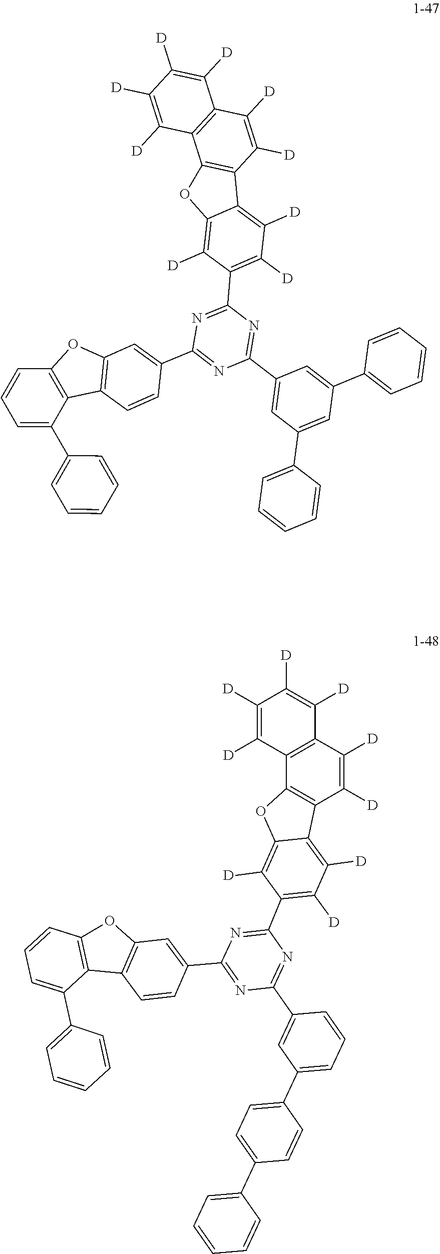 [SMILES: [2H]c1c([2H])c([2H])c2c(c1[2H])c([2H])c([2H])c1c2oc2c([2H])c(-c3nc(-c4cc(-c5ccccc5)cc(-c5ccccc5)c4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4cccc(-c5ccc(-c6ccccc6)cc5)c4)nc(-c4ccc5c(c4)oc4cccc(-c6ccccc6)c45)n3)c([2H])c([2H])c21]

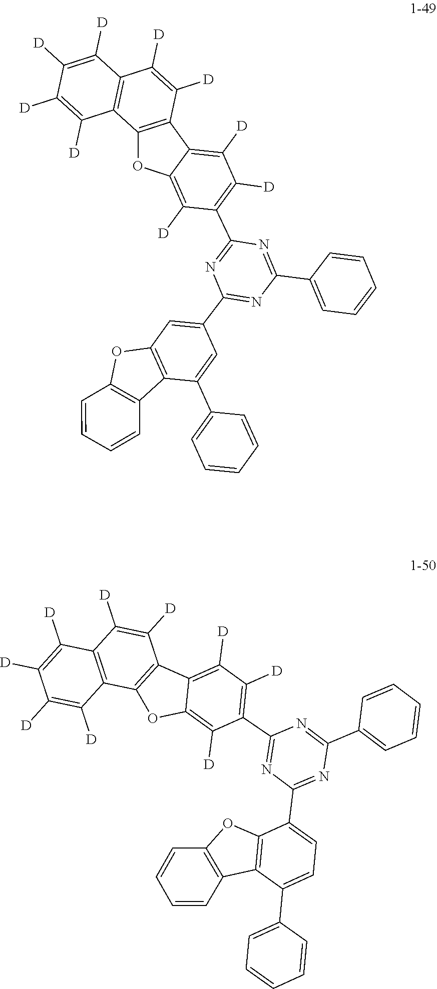 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)c5c(c4)oc4ccccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc(-c5ccccc5)c5c4oc4ccccc45)n3)c([2H])c([2H])c21]

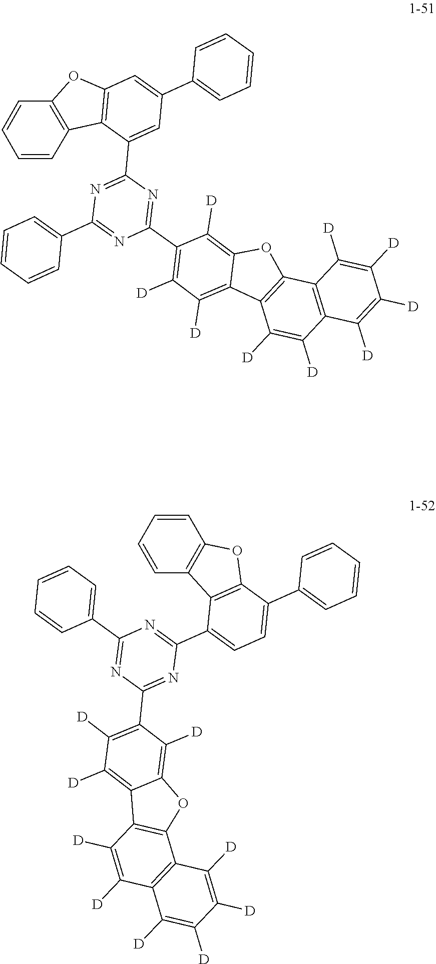 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)cc5oc6ccccc6c45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc(-c5ccccc5)c5oc6ccccc6c45)n3)c([2H])c([2H])c21]

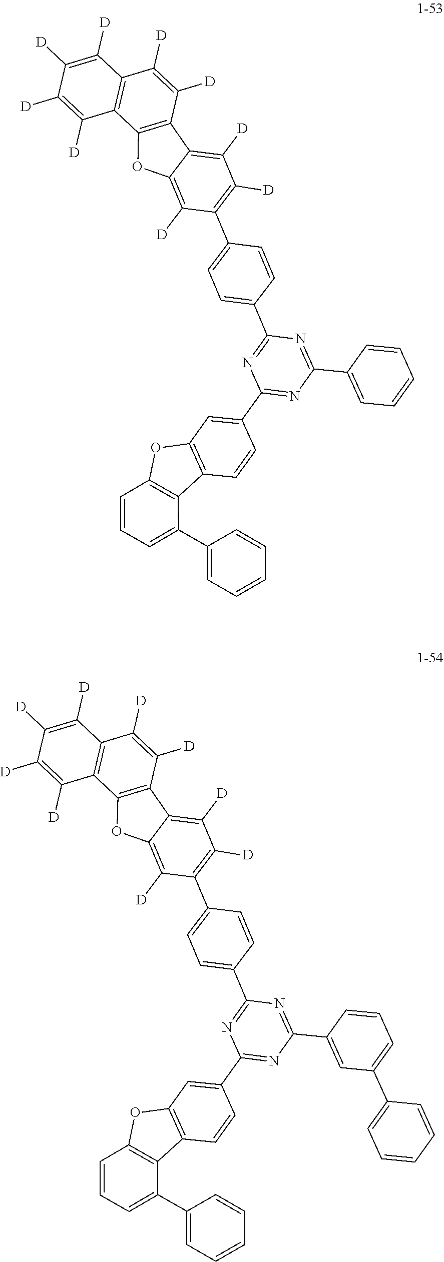 [SMILES: [2H]c1c([2H])c([2H])c2c(c1[2H])c([2H])c([2H])c1c2oc2c([2H])c(-c3ccc(-c4nc(-c5ccccc5)nc(-c5ccc6c(c5)oc5cccc(-c7ccccc7)c56)n4)cc3)c([2H])c([2H])c21 ; [2H]c1c([2H])c([2H])c2c(c1[2H])c([2H])c([2H])c1c2oc2c([2H])c(-c3ccc(-c4nc(-c5cccc(-c6ccccc6)c5)nc(-c5ccc6c(c5)oc5cccc(-c7ccccc7)c56)n4)cc3)c([2H])c([2H])c21]

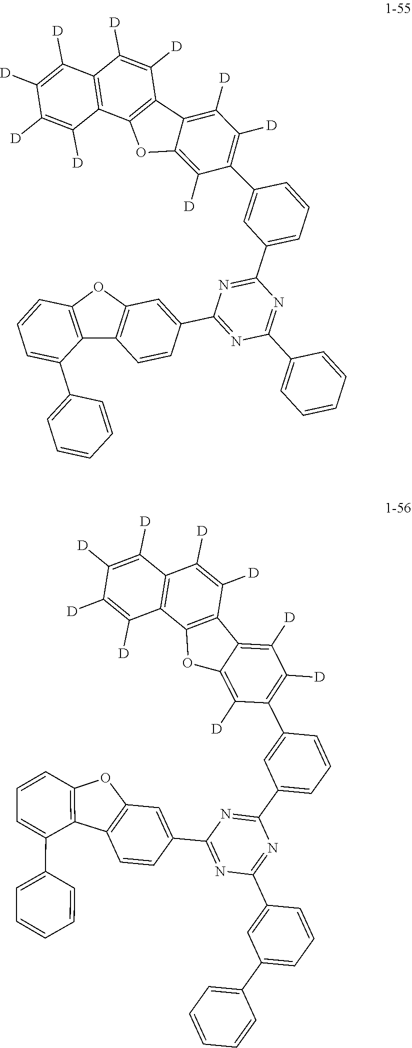 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)oc5cccc(-c7ccccc7)c56)n4)c3)c([2H])c([2H])c21 ; [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)oc5cccc(-c7ccccc7)c56)n4)c3)c([2H])c([2H])c21]

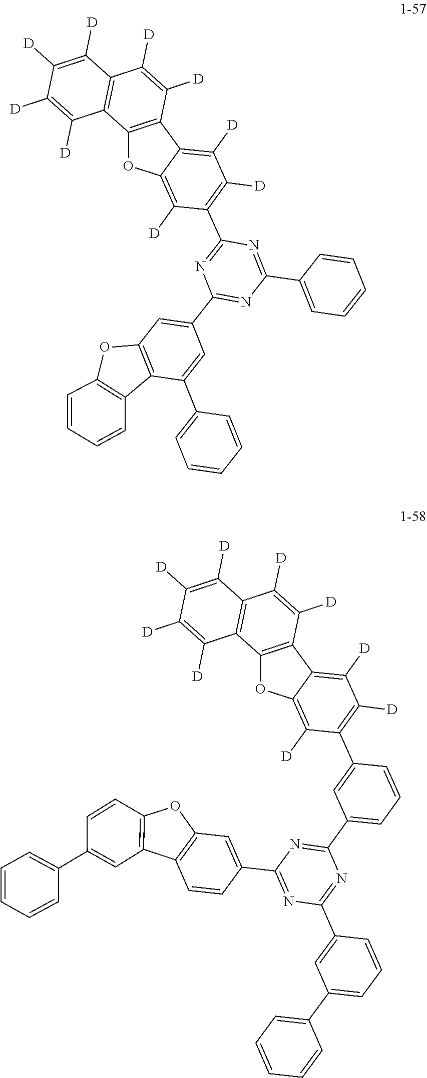 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)c5c(c4)oc4ccccc45)n3)c([2H])c([2H])c21 ; [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)oc5ccc(-c7ccccc7)cc56)n4)c3)c([2H])c([2H])c21]

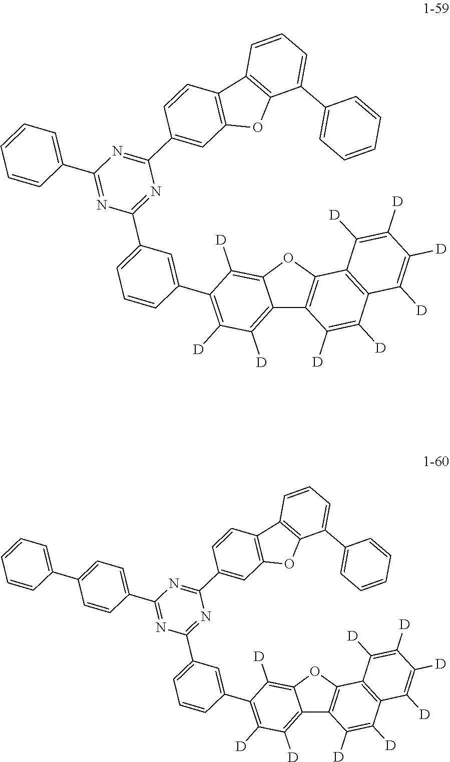 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)oc5c(-c7ccccc7)cccc56)n4)c3)c([2H])c([2H])c21 ; [2H]c1c([2H])c([2H])c2c(c1[2H])c([2H])c([2H])c1c2oc2c([2H])c(-c3cccc(-c4nc(-c5ccc(-c6ccccc6)cc5)nc(-c5ccc6c(c5)oc5c(-c7ccccc7)cccc56)n4)c3)c([2H])c([2H])c21]

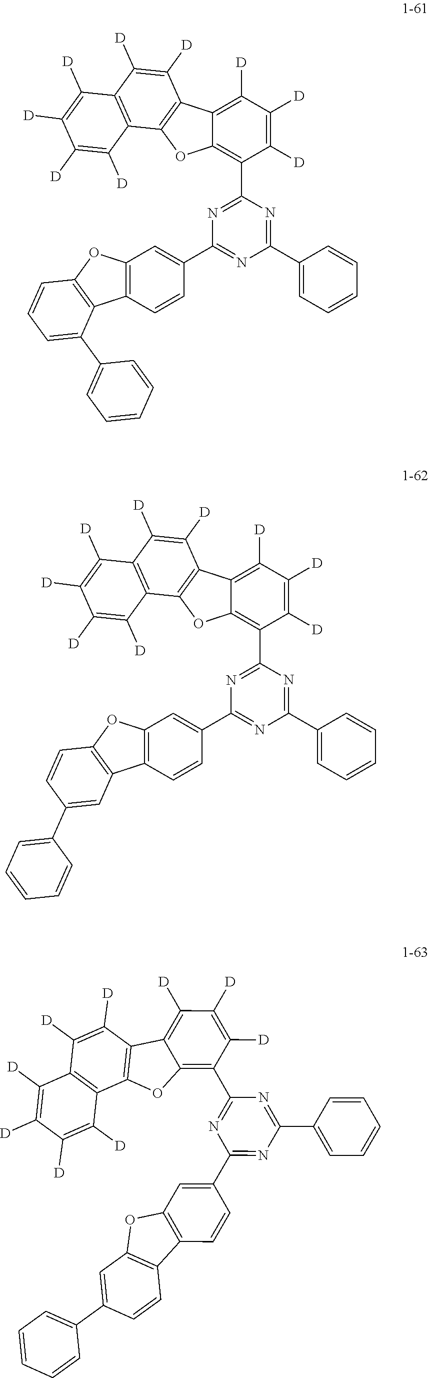 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2ccc(-c4ccccc4)cc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2cc(-c4ccccc4)ccc23)n1]

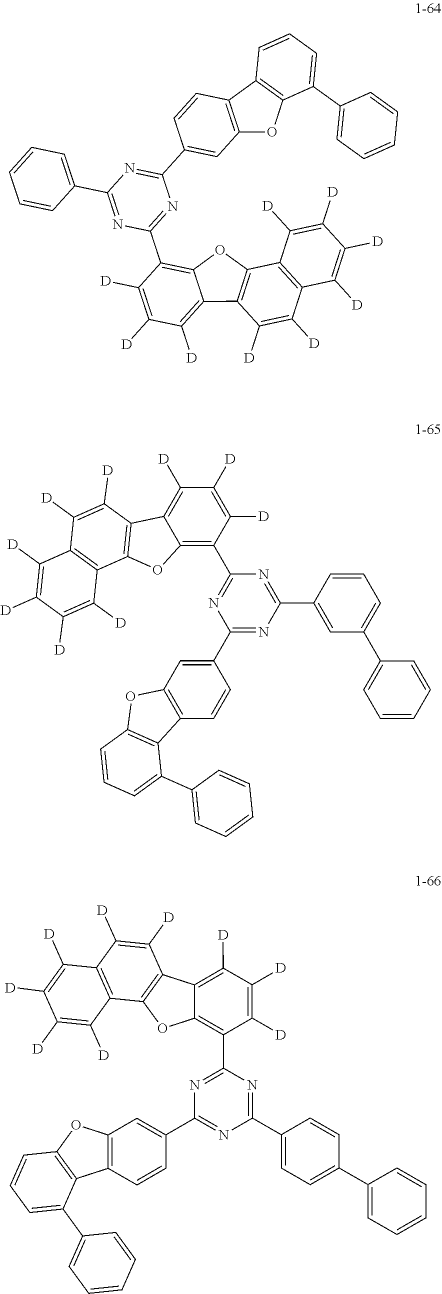 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2c(-c4ccccc4)cccc23)n1 ; [2H]c1c([2H])c([2H])c2c(oc3c4c([2H])c([2H])c([2H])c([2H])c4c([2H])c([2H])c32)c1-c1nc(-c2cccc(-c3ccccc3)c2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccc(-c3ccccc3)cc2)nc(-c2ccc3c(c2)oc2cccc(-c4ccccc4)c23)n1]

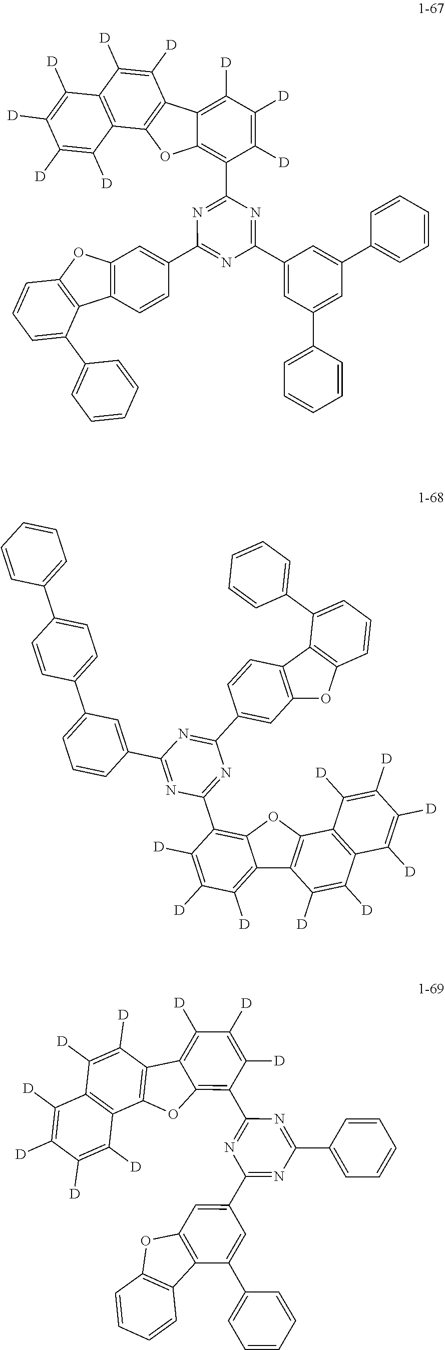 [SMILES: [2H]c1c([2H])c([2H])c2c(oc3c4c([2H])c([2H])c([2H])c([2H])c4c([2H])c([2H])c32)c1-c1nc(-c2cc(-c3ccccc3)cc(-c3ccccc3)c2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2cccc(-c3ccc(-c4ccccc4)cc3)c2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)c3c(c2)oc2ccccc23)n1]

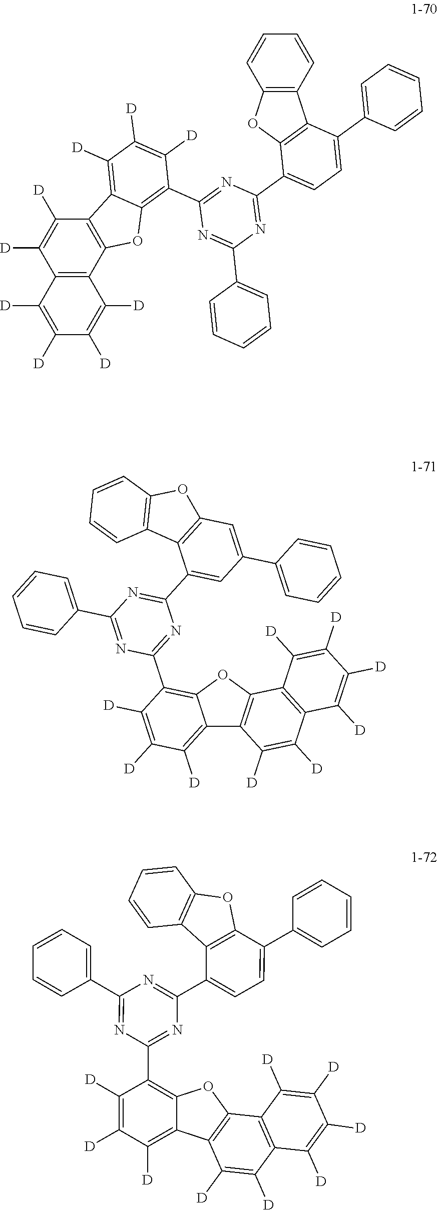 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3c2oc2ccccc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)cc3oc4ccccc4c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3oc4ccccc4c23)n1]

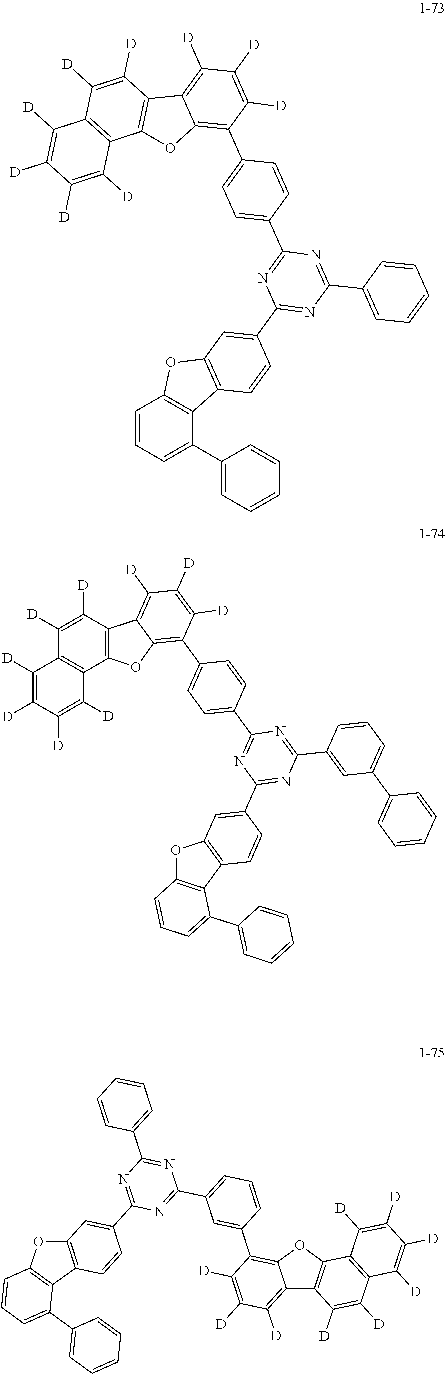 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3ccc4c(c3)oc3cccc(-c5ccccc5)c34)n2)cc1 ; [2H]c1c([2H])c([2H])c2c(oc3c4c([2H])c([2H])c([2H])c([2H])c4c([2H])c([2H])c32)c1-c1ccc(-c2nc(-c3cccc(-c4ccccc4)c3)nc(-c3ccc4c(c3)oc3cccc(-c5ccccc5)c34)n2)cc1 ; [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)oc3cccc(-c5ccccc5)c34)n2)c1]

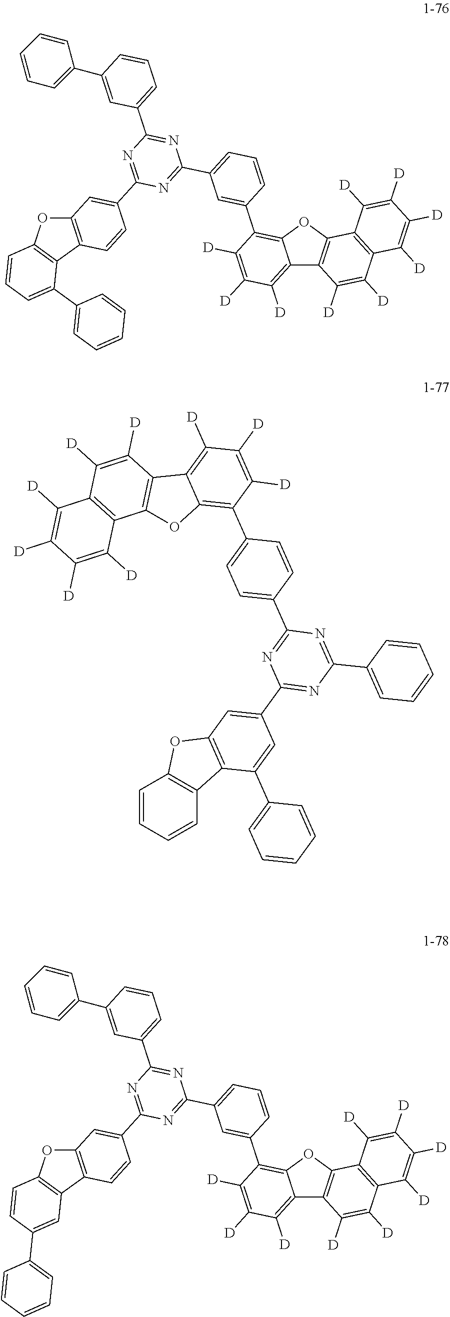 [SMILES: [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3cccc(-c5ccccc5)c34)n2)c1 ; [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3cc(-c4ccccc4)c4c(c3)oc3ccccc34)n2)cc1 ; [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3ccc(-c5ccccc5)cc34)n2)c1]

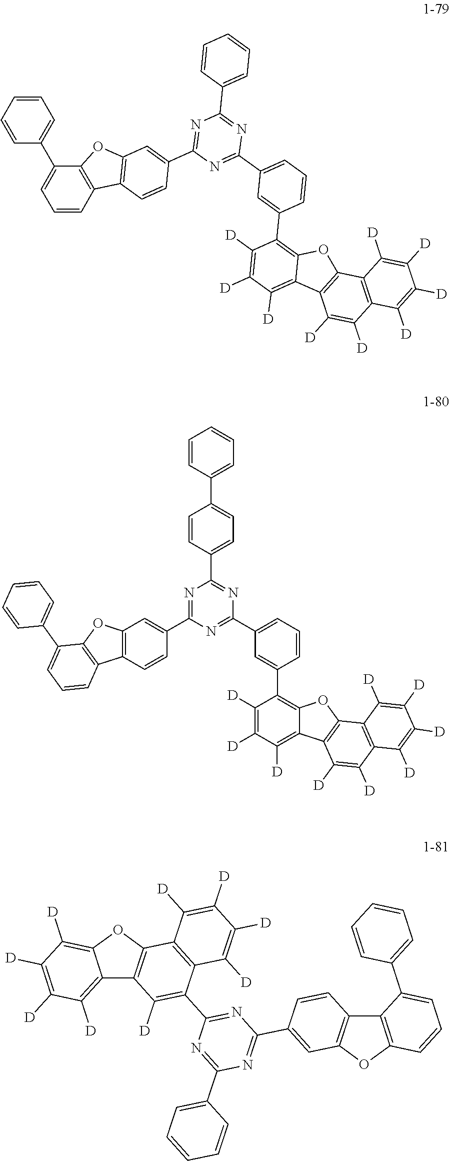 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)oc3c(-c5ccccc5)cccc34)n2)c1 ; [2H]c1c([2H])c([2H])c2c(oc3c4c([2H])c([2H])c([2H])c([2H])c4c([2H])c([2H])c32)c1-c1cccc(-c2nc(-c3ccc(-c4ccccc4)cc3)nc(-c3ccc4c(c3)oc3c(-c5ccccc5)cccc34)n2)c1 ; [2H]c1c([2H])c([2H])c2c(oc3c4c([2H])c([2H])c([2H])c([2H])c4c(-c4nc(-c5ccccc5)nc(-c5ccc6c(c5)oc5cccc(-c7ccccc7)c56)n4)c([2H])c32)c1[2H]]

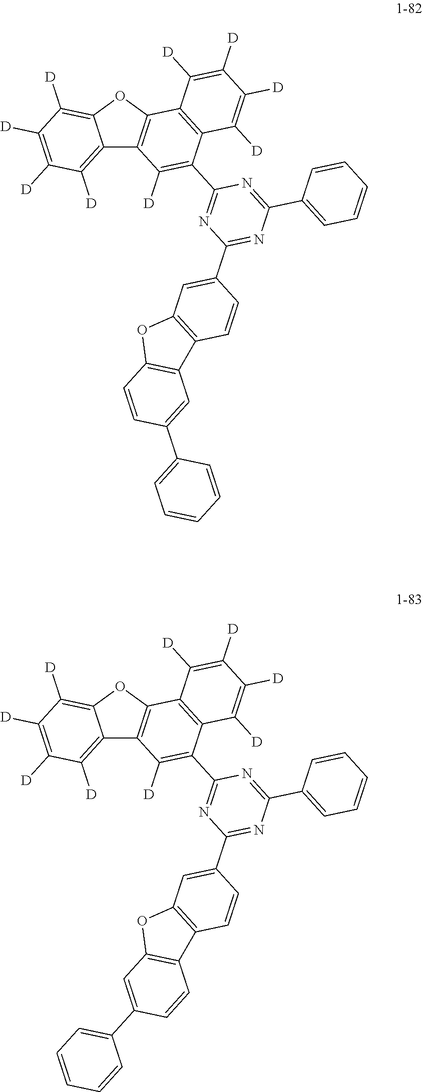 [SMILES: [2H]c1c([2H])c([2H])c2c(oc3c4c([2H])c([2H])c([2H])c([2H])c4c(-c4nc(-c5ccccc5)nc(-c5ccc6c(c5)oc5ccc(-c7ccccc7)cc56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc6c(c5)oc5cc(-c7ccccc7)ccc56)n4)c([2H])c32)c1[2H]]

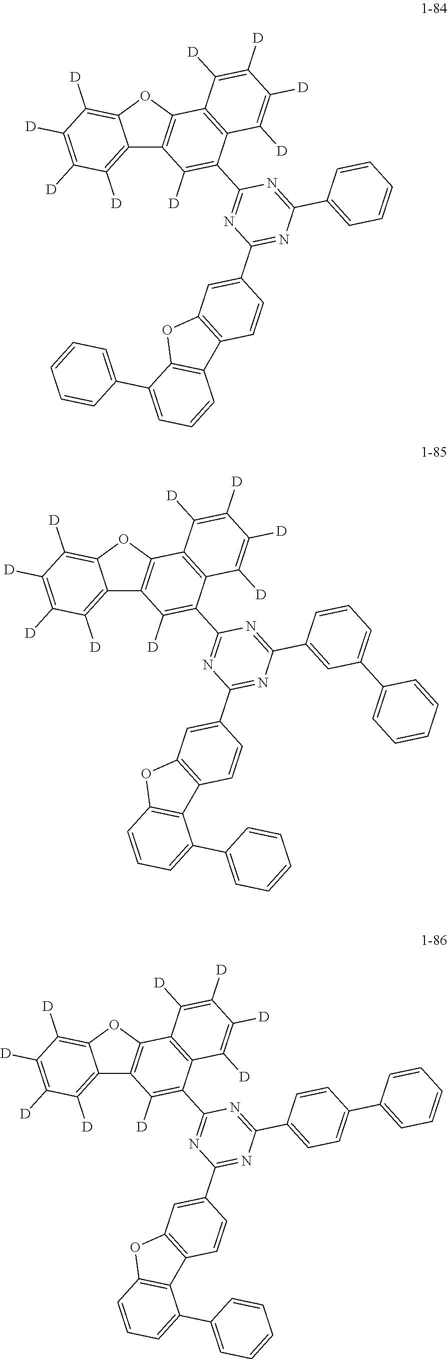 [SMILES: [2H]c1c([2H])c([2H])c2c(oc3c4c([2H])c([2H])c([2H])c([2H])c4c(-c4nc(-c5ccccc5)nc(-c5ccc6c(c5)oc5c(-c7ccccc7)cccc56)n4)c([2H])c32)c1[2H] ; [2H]c1c([2H])c([2H])c2c(oc3c4c([2H])c([2H])c([2H])c([2H])c4c(-c4nc(-c5cccc(-c6ccccc6)c5)nc(-c5ccc6c(c5)oc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5ccc(-c6ccccc6)cc5)nc(-c5ccc6c(c5)oc5cccc(-c7ccccc7)c56)n4)c([2H])c32)c1[2H]]

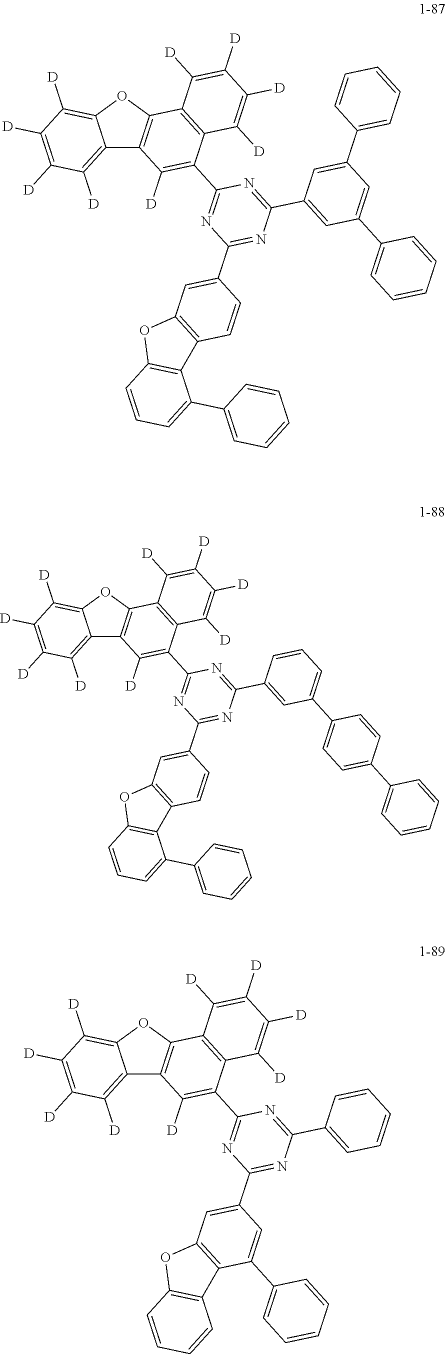 [SMILES: [2H]c1c([2H])c([2H])c2c(oc3c4c([2H])c([2H])c([2H])c([2H])c4c(-c4nc(-c5cc(-c6ccccc6)cc(-c6ccccc6)c5)nc(-c5ccc6c(c5)oc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5cccc(-c6ccc(-c7ccccc7)cc6)c5)nc(-c5ccc6c(c5)oc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5cc(-c6ccccc6)c6c(c5)oc5ccccc56)n4)c([2H])c32)c1[2H]]

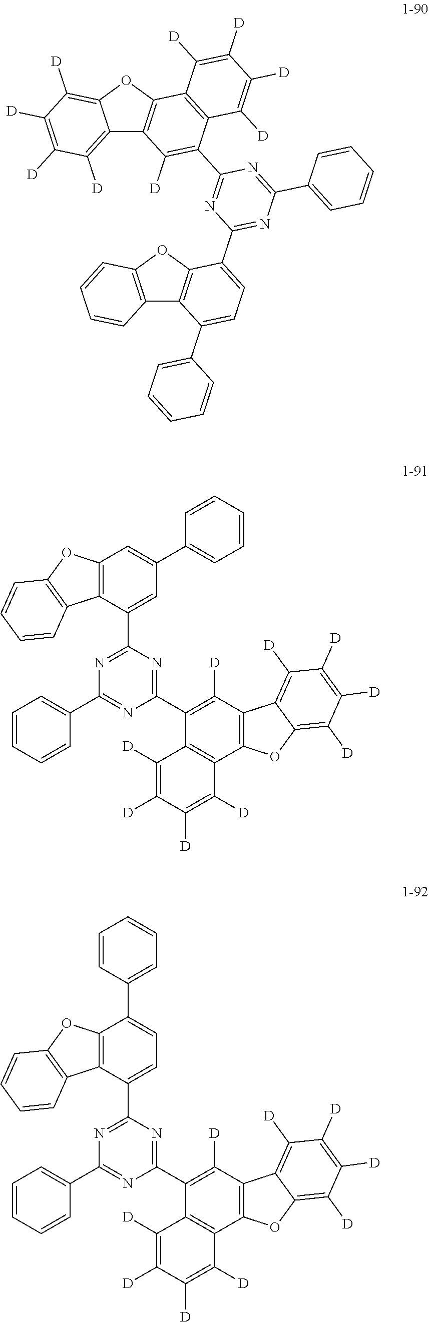 [SMILES: [2H]c1c([2H])c([2H])c2c(oc3c4c([2H])c([2H])c([2H])c([2H])c4c(-c4nc(-c5ccccc5)nc(-c5ccc(-c6ccccc6)c6c5oc5ccccc56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5cc(-c6ccccc6)cc6oc7ccccc7c56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc(-c6ccccc6)c6oc7ccccc7c56)n4)c([2H])c32)c1[2H]]

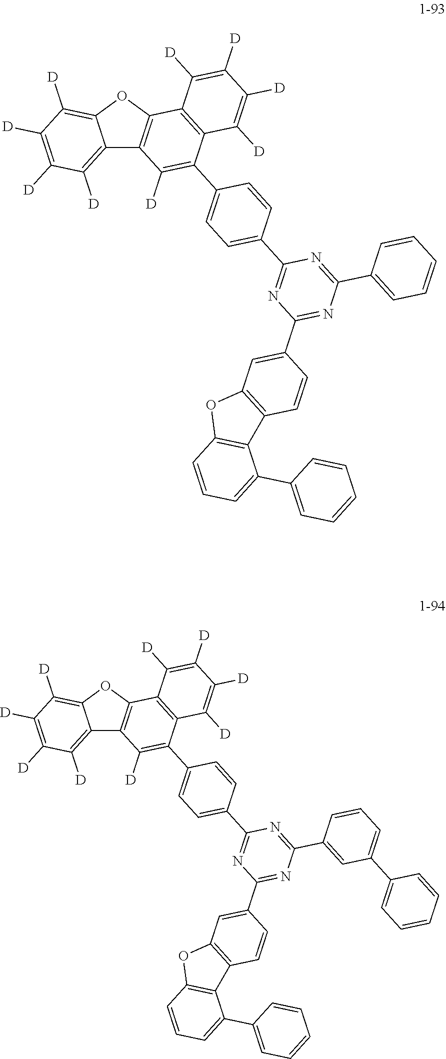 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]]

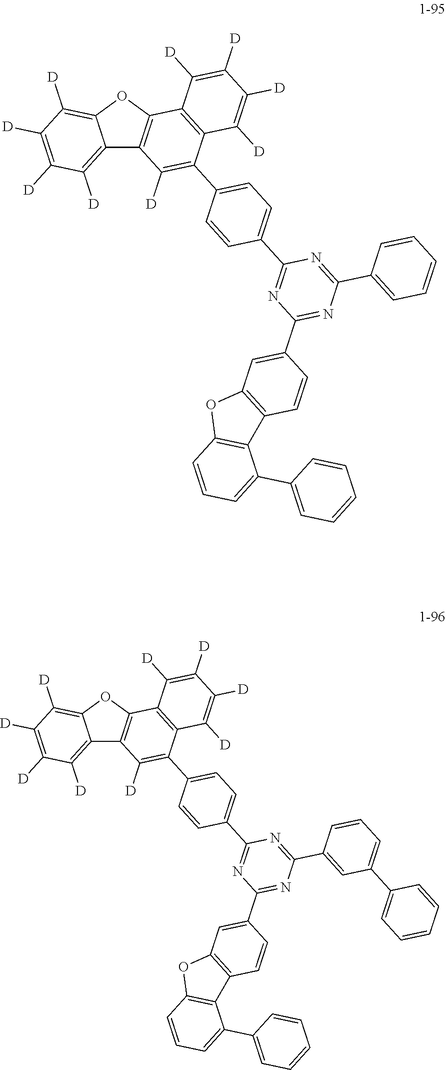 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]]

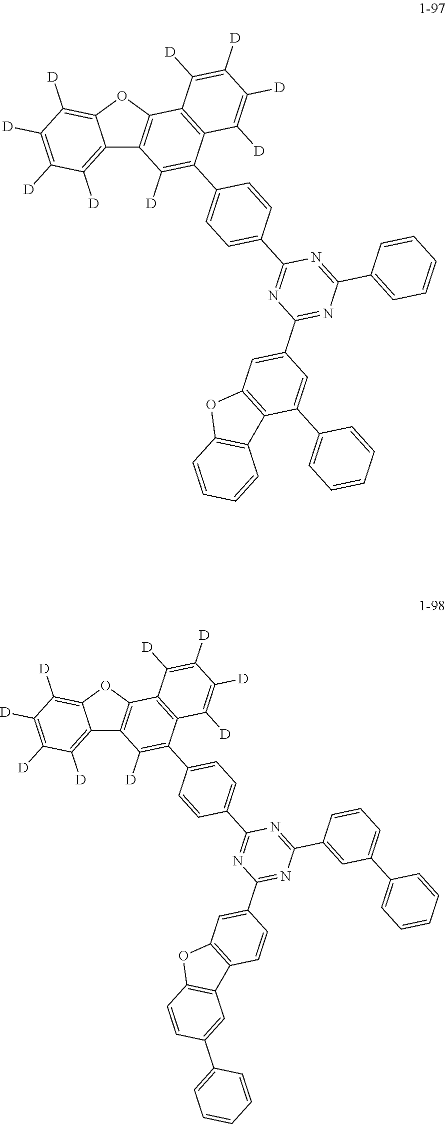 [SMILES: [2H]c1c([2H])c([2H])c2c(oc3c4c([2H])c([2H])c([2H])c([2H])c4c(-c4ccc(-c5nc(-c6ccccc6)nc(-c6cc(-c7ccccc7)c7c(c6)oc6ccccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6ccc(-c8ccccc8)cc67)n5)cc4)c([2H])c32)c1[2H]]

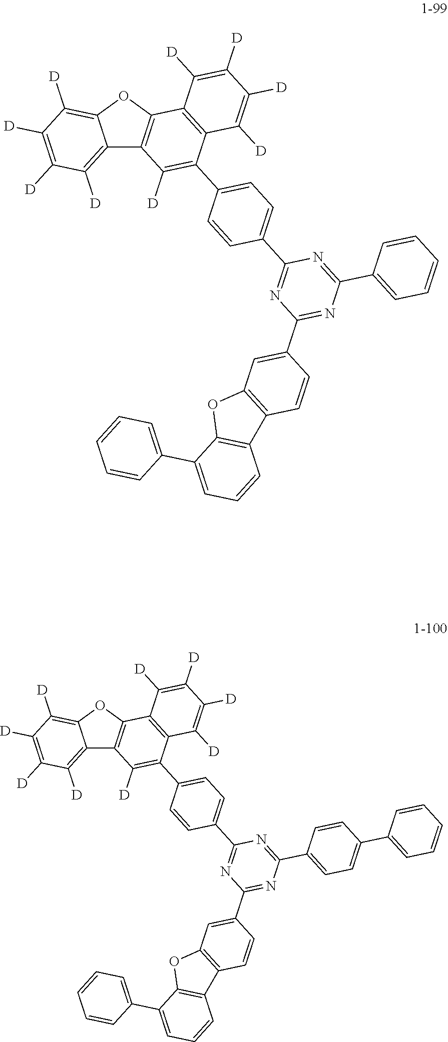 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6c(-c8ccccc8)cccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6ccc(-c7ccccc7)cc6)nc(-c6ccc7c(c6)oc6c(-c8ccccc8)cccc67)n5)cc4)c([2H])c32)c1[2H]]

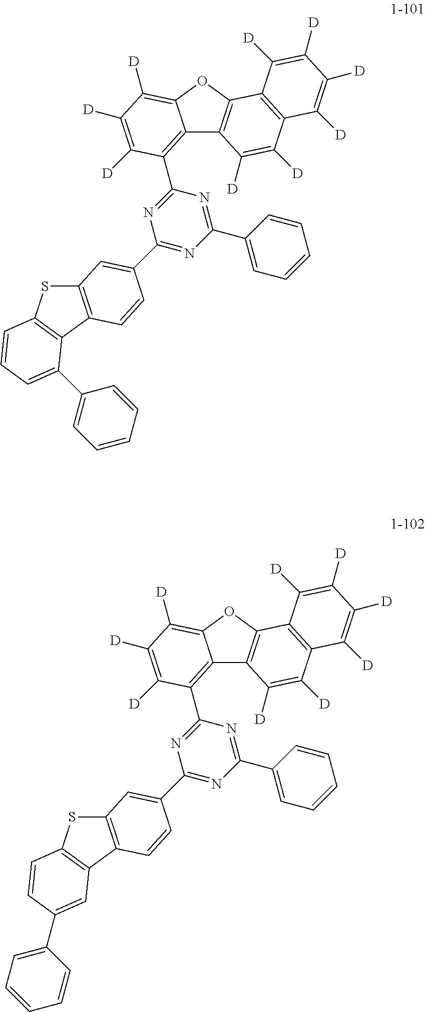 [SMILES: [2H]c1c([2H])c(-c2nc(-c3ccccc3)nc(-c3ccc4c(c3)sc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)sc3ccc(-c5ccccc5)cc34)n2)c2c(oc3c4c([2H])c([2H])c([2H])c([2H])c4c([2H])c([2H])c32)c1[2H]]

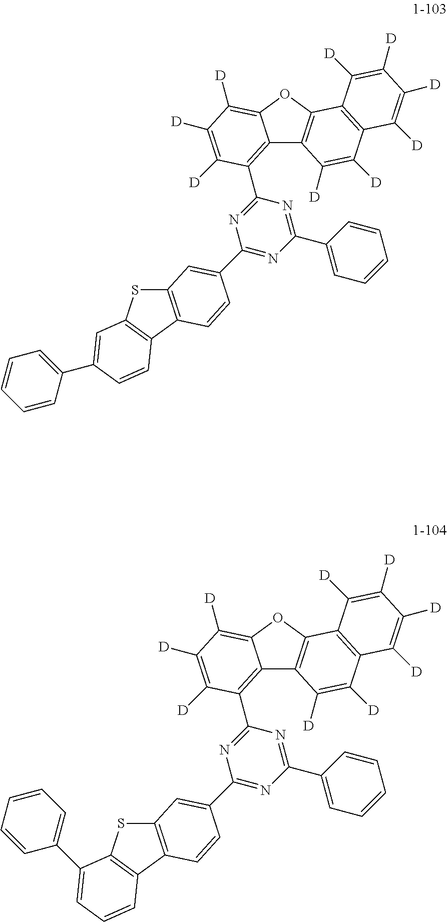 [SMILES: [2H]c1c([2H])c(-c2nc(-c3ccccc3)nc(-c3ccc4c(c3)sc3cc(-c5ccccc5)ccc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)sc3c(-c5ccccc5)cccc34)n2)c2c(oc3c4c([2H])c([2H])c([2H])c([2H])c4c([2H])c([2H])c32)c1[2H]]

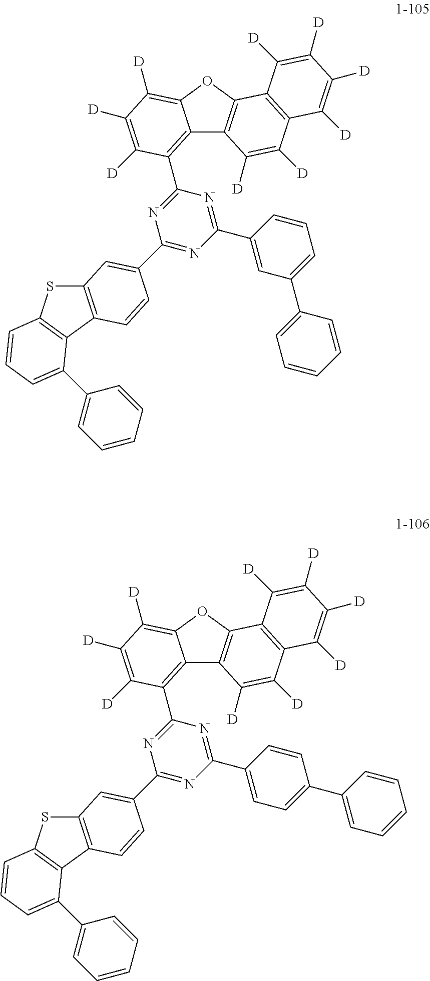 [SMILES: [2H]c1c([2H])c(-c2nc(-c3cccc(-c4ccccc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccc(-c4ccccc4)cc3)nc(-c3ccc4c(c3)sc3cccc(-c5ccccc5)c34)n2)c2c(oc3c4c([2H])c([2H])c([2H])c([2H])c4c([2H])c([2H])c32)c1[2H]]

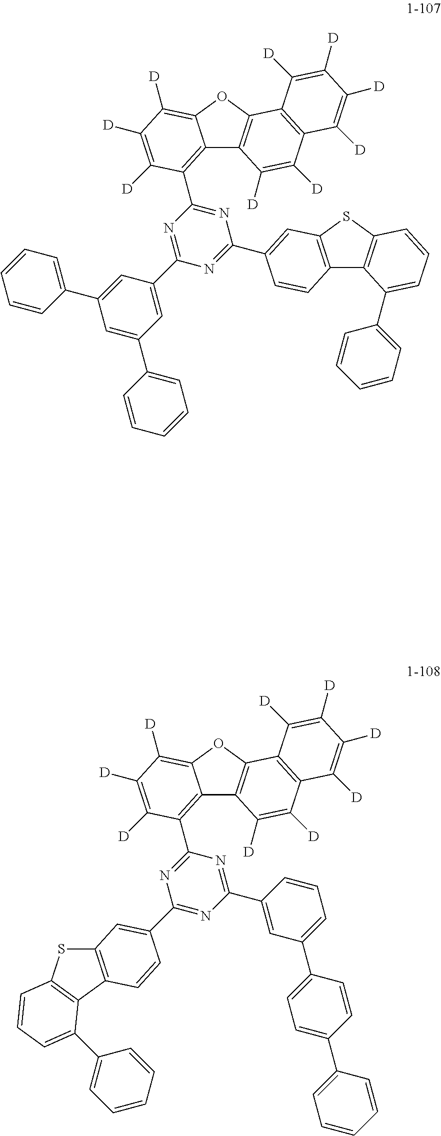 [SMILES: [2H]c1c([2H])c(-c2nc(-c3cc(-c4ccccc4)cc(-c4ccccc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3cccc(-c4ccc(-c5ccccc5)cc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H]]

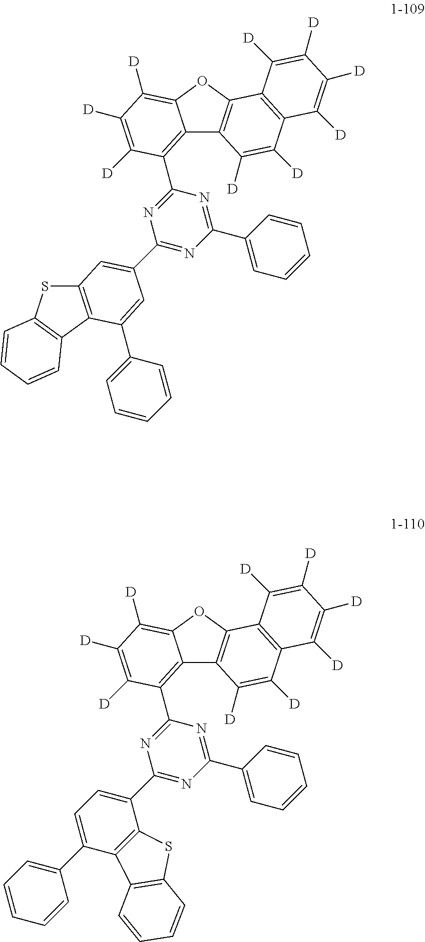 [SMILES: [2H]c1c([2H])c(-c2nc(-c3ccccc3)nc(-c3cc(-c4ccccc4)c4c(c3)sc3ccccc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc(-c4ccccc4)c4c3sc3ccccc34)n2)c2c(oc3c4c([2H])c([2H])c([2H])c([2H])c4c([2H])c([2H])c32)c1[2H]]

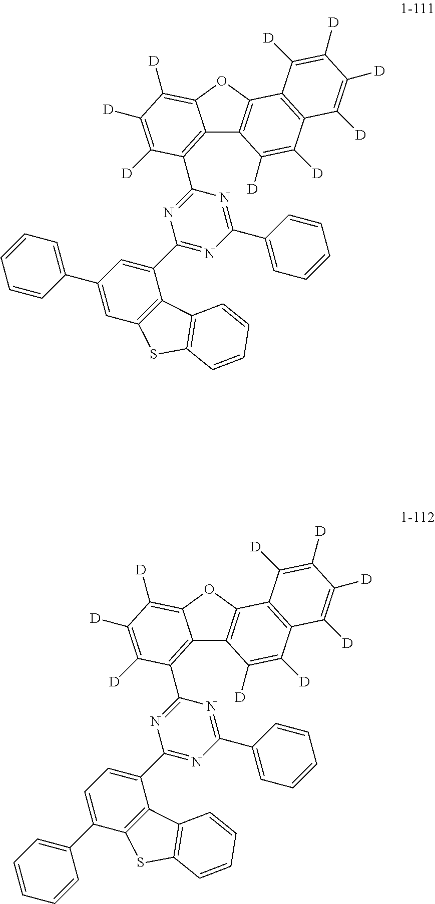 [SMILES: [2H]c1c([2H])c(-c2nc(-c3ccccc3)nc(-c3cc(-c4ccccc4)cc4sc5ccccc5c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc(-c4ccccc4)c4sc5ccccc5c34)n2)c2c(oc3c4c([2H])c([2H])c([2H])c([2H])c4c([2H])c([2H])c32)c1[2H]]

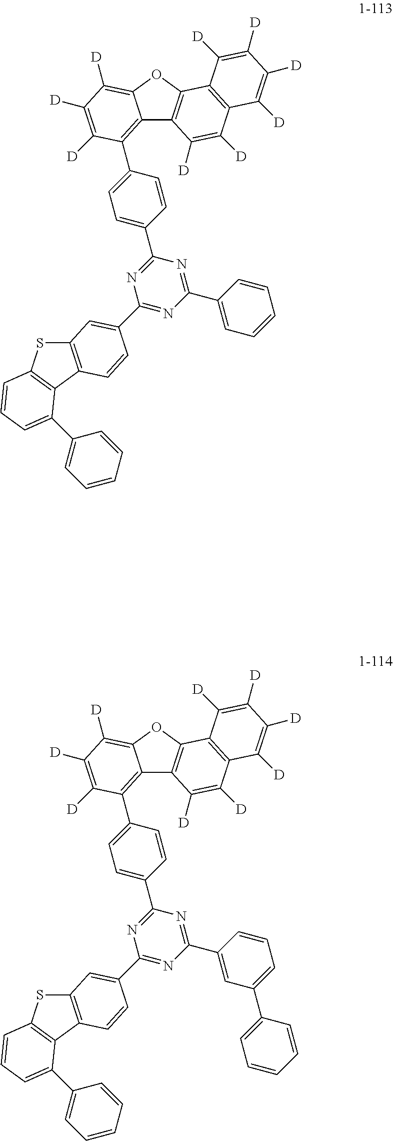 [SMILES: [2H]c1c([2H])c(-c2ccc(-c3nc(-c4ccccc4)nc(-c4ccc5c(c4)sc4cccc(-c6ccccc6)c45)n3)cc2)c2c(oc3c4c([2H])c([2H])c([2H])c([2H])c4c([2H])c([2H])c32)c1[2H] ; [2H]c1c([2H])c(-c2ccc(-c3nc(-c4cccc(-c5ccccc5)c4)nc(-c4ccc5c(c4)sc4cccc(-c6ccccc6)c45)n3)cc2)c2c(oc3c4c([2H])c([2H])c([2H])c([2H])c4c([2H])c([2H])c32)c1[2H]]

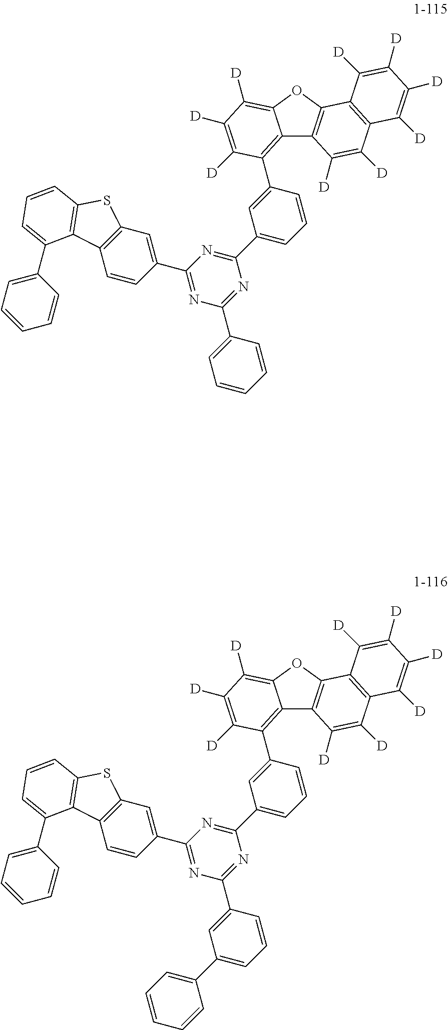 [SMILES: [2H]c1c([2H])c(-c2cccc(-c3nc(-c4ccccc4)nc(-c4ccc5c(c4)sc4cccc(-c6ccccc6)c45)n3)c2)c2c(oc3c4c([2H])c([2H])c([2H])c([2H])c4c([2H])c([2H])c32)c1[2H] ; [2H]c1c([2H])c(-c2cccc(-c3nc(-c4cccc(-c5ccccc5)c4)nc(-c4ccc5c(c4)sc4cccc(-c6ccccc6)c45)n3)c2)c2c(oc3c4c([2H])c([2H])c([2H])c([2H])c4c([2H])c([2H])c32)c1[2H]]

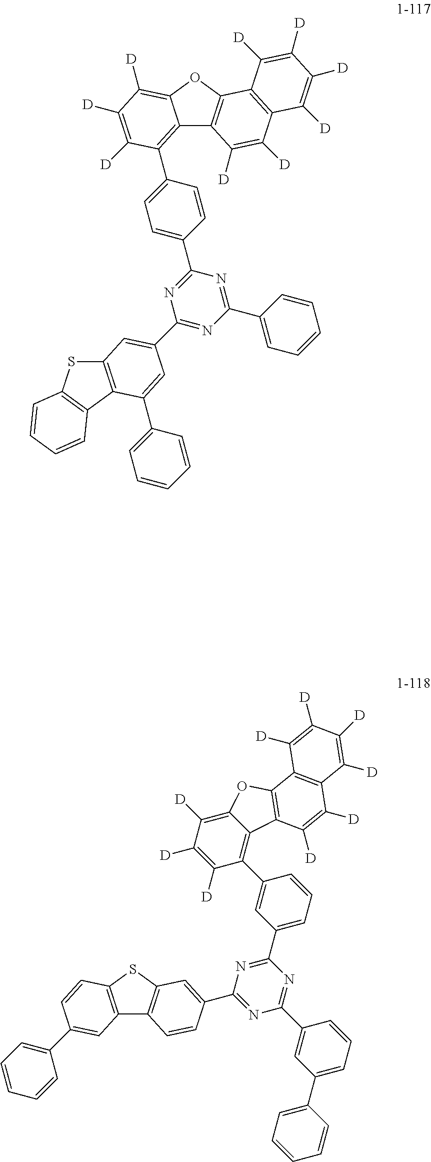 [SMILES: [2H]c1c([2H])c(-c2ccc(-c3nc(-c4ccccc4)nc(-c4cc(-c5ccccc5)c5c(c4)sc4ccccc45)n3)cc2)c2c(oc3c4c([2H])c([2H])c([2H])c([2H])c4c([2H])c([2H])c32)c1[2H] ; [2H]c1c([2H])c(-c2cccc(-c3nc(-c4cccc(-c5ccccc5)c4)nc(-c4ccc5c(c4)sc4ccc(-c6ccccc6)cc45)n3)c2)c2c(oc3c4c([2H])c([2H])c([2H])c([2H])c4c([2H])c([2H])c32)c1[2H]]

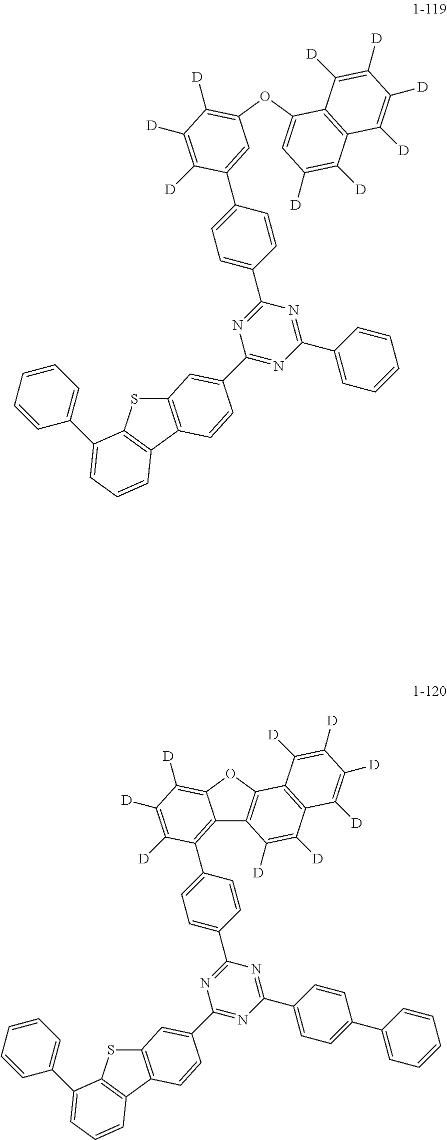 [SMILES: [2H]c1cc(Oc2cc(-c3ccc(-c4nc(-c5ccccc5)nc(-c5ccc6c(c5)sc5c(-c7ccccc7)cccc56)n4)cc3)c([2H])c([2H])c2[2H])c2c([2H])c([2H])c([2H])c([2H])c2c1[2H] ; [2H]c1c([2H])c(-c2ccc(-c3nc(-c4ccc(-c5ccccc5)cc4)nc(-c4ccc5c(c4)sc4c(-c6ccccc6)cccc45)n3)cc2)c2c(oc3c4c([2H])c([2H])c([2H])c([2H])c4c([2H])c([2H])c32)c1[2H]]

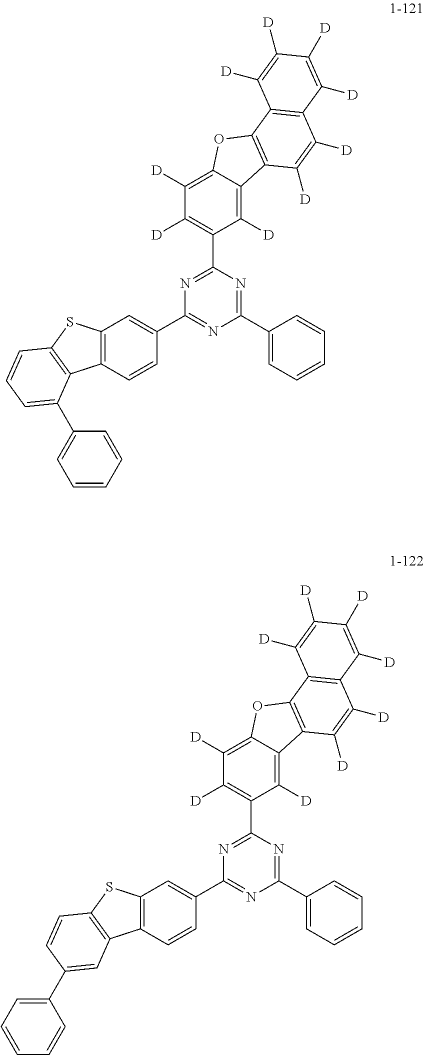 [SMILES: [2H]c1c(-c2nc(-c3ccccc3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)sc3ccc(-c5ccccc5)cc34)n2)c([2H])c2c(oc3c4c([2H])c([2H])c([2H])c([2H])c4c([2H])c([2H])c32)c1[2H]]

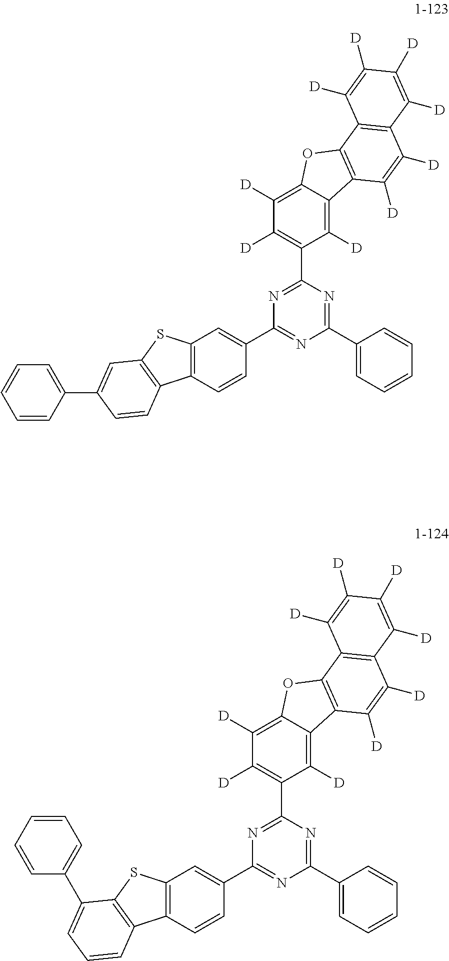 [SMILES: [2H]c1c(-c2nc(-c3ccccc3)nc(-c3ccc4c(c3)sc3cc(-c5ccccc5)ccc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)sc3c(-c5ccccc5)cccc34)n2)c([2H])c2c(oc3c4c([2H])c([2H])c([2H])c([2H])c4c([2H])c([2H])c32)c1[2H]]

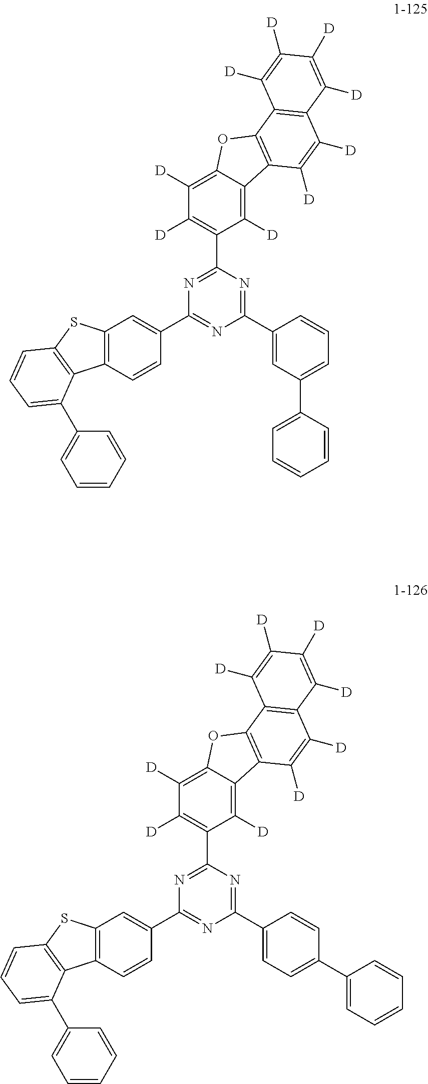 [SMILES: [2H]c1c(-c2nc(-c3cccc(-c4ccccc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3ccc(-c4ccccc4)cc3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]]

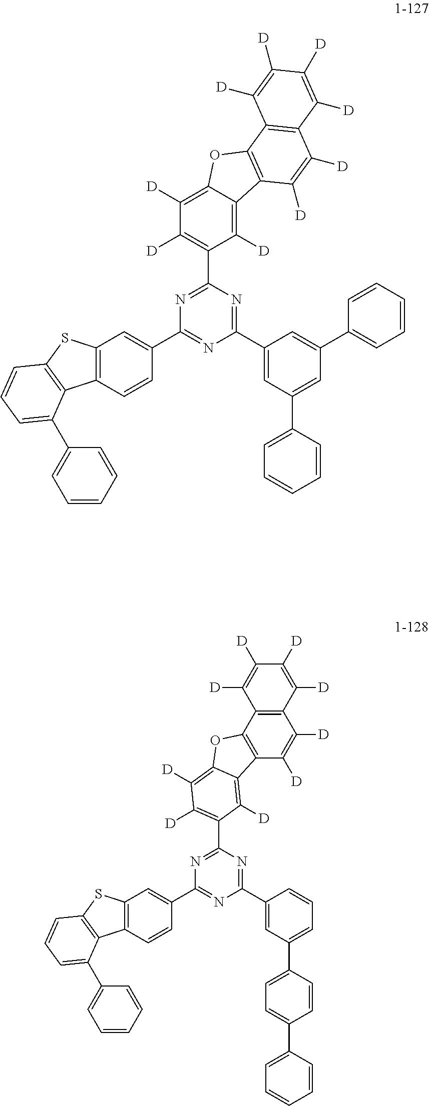 [SMILES: [2H]c1c(-c2nc(-c3cc(-c4ccccc4)cc(-c4ccccc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3cccc(-c4ccc(-c5ccccc5)cc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]]

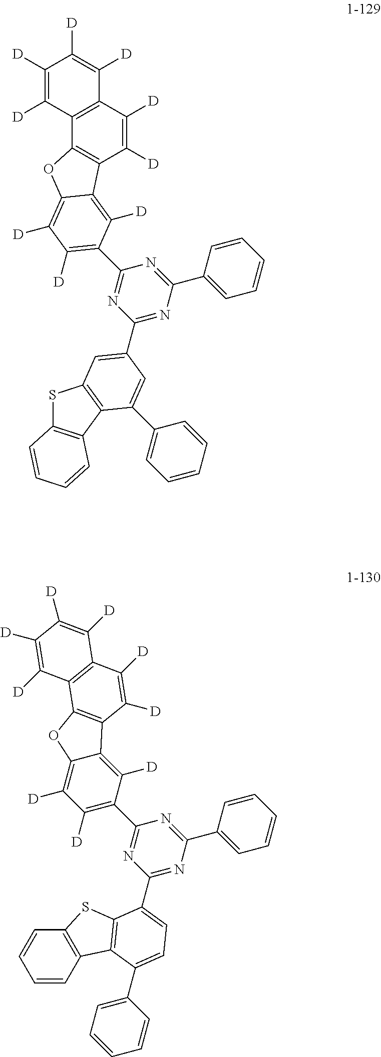 [SMILES: [2H]c1c(-c2nc(-c3ccccc3)nc(-c3cc(-c4ccccc4)c4c(c3)sc3ccccc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc(-c4ccccc4)c4c3sc3ccccc34)n2)c([2H])c2c(oc3c4c([2H])c([2H])c([2H])c([2H])c4c([2H])c([2H])c32)c1[2H]]

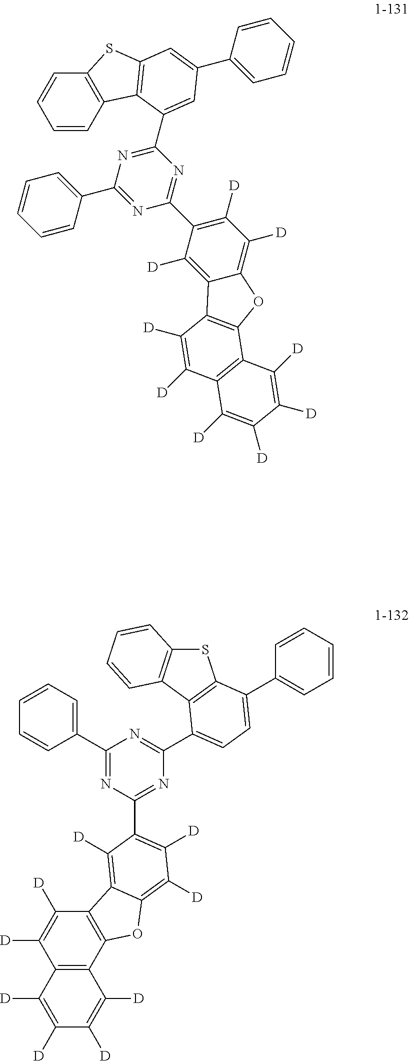 [SMILES: [2H]c1c(-c2nc(-c3ccccc3)nc(-c3cc(-c4ccccc4)cc4sc5ccccc5c34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc(-c4ccccc4)c4sc5ccccc5c34)n2)c([2H])c2c(oc3c4c([2H])c([2H])c([2H])c([2H])c4c([2H])c([2H])c32)c1[2H]]

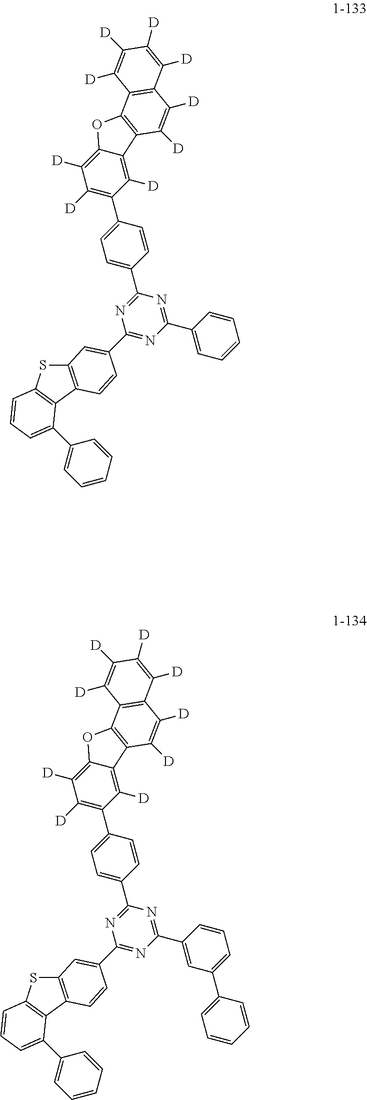 [SMILES: [2H]c1c(-c2ccc(-c3nc(-c4ccccc4)nc(-c4ccc5c(c4)sc4cccc(-c6ccccc6)c45)n3)cc2)c([2H])c2c(oc3c4c([2H])c([2H])c([2H])c([2H])c4c([2H])c([2H])c32)c1[2H] ; [2H]c1c(-c2ccc(-c3nc(-c4cccc(-c5ccccc5)c4)nc(-c4ccc5c(c4)sc4cccc(-c6ccccc6)c45)n3)cc2)c([2H])c2c(oc3c4c([2H])c([2H])c([2H])c([2H])c4c([2H])c([2H])c32)c1[2H]]

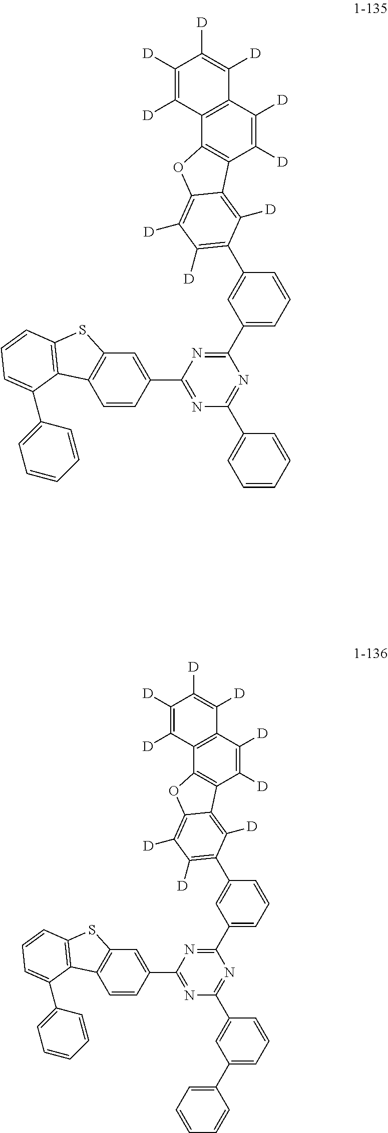 [SMILES: [2H]c1c(-c2cccc(-c3nc(-c4ccccc4)nc(-c4ccc5c(c4)sc4cccc(-c6ccccc6)c45)n3)c2)c([2H])c2c(oc3c4c([2H])c([2H])c([2H])c([2H])c4c([2H])c([2H])c32)c1[2H] ; [2H]c1c(-c2cccc(-c3nc(-c4cccc(-c5ccccc5)c4)nc(-c4ccc5c(c4)sc4cccc(-c6ccccc6)c45)n3)c2)c([2H])c2c(oc3c4c([2H])c([2H])c([2H])c([2H])c4c([2H])c([2H])c32)c1[2H]]

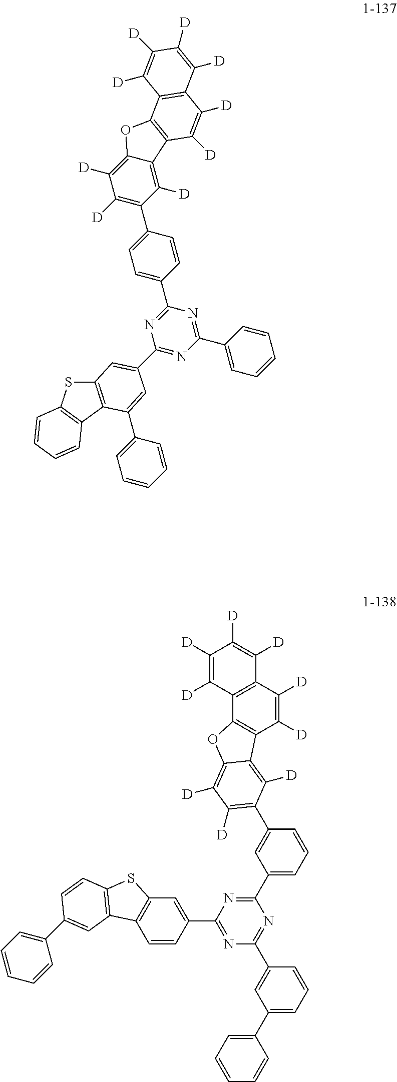 [SMILES: [2H]c1c(-c2ccc(-c3nc(-c4ccccc4)nc(-c4cc(-c5ccccc5)c5c(c4)sc4ccccc45)n3)cc2)c([2H])c2c(oc3c4c([2H])c([2H])c([2H])c([2H])c4c([2H])c([2H])c32)c1[2H] ; [2H]c1c(-c2cccc(-c3nc(-c4cccc(-c5ccccc5)c4)nc(-c4ccc5c(c4)sc4ccc(-c6ccccc6)cc45)n3)c2)c([2H])c2c(oc3c4c([2H])c([2H])c([2H])c([2H])c4c([2H])c([2H])c32)c1[2H]]

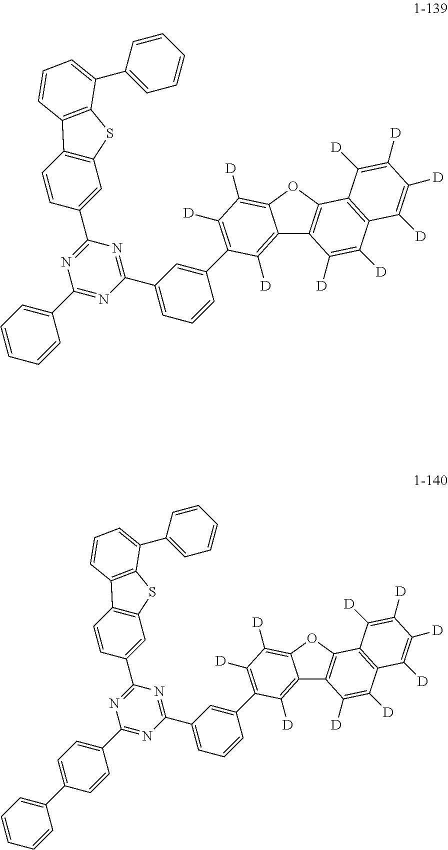 [SMILES: [2H]c1c(-c2cccc(-c3nc(-c4ccccc4)nc(-c4ccc5c(c4)sc4c(-c6ccccc6)cccc45)n3)c2)c([2H])c2c(oc3c4c([2H])c([2H])c([2H])c([2H])c4c([2H])c([2H])c32)c1[2H] ; [2H]c1c(-c2cccc(-c3nc(-c4ccc(-c5ccccc5)cc4)nc(-c4ccc5c(c4)sc4c(-c6ccccc6)cccc45)n3)c2)c([2H])c2c(oc3c4c([2H])c([2H])c([2H])c([2H])c4c([2H])c([2H])c32)c1[2H]]

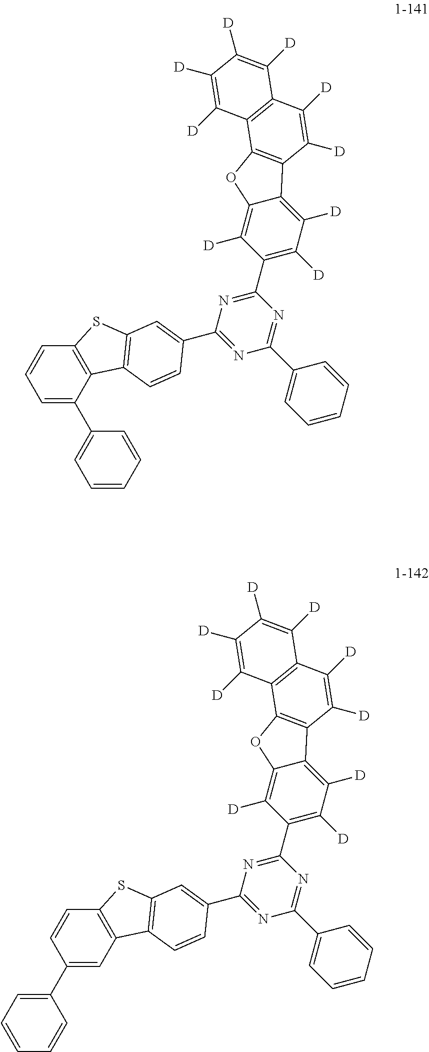 [SMILES: [2H]c1c([2H])c([2H])c2c(c1[2H])c([2H])c([2H])c1c2oc2c([2H])c(-c3nc(-c4ccccc4)nc(-c4ccc5c(c4)sc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)sc4ccc(-c6ccccc6)cc45)n3)c([2H])c([2H])c21]

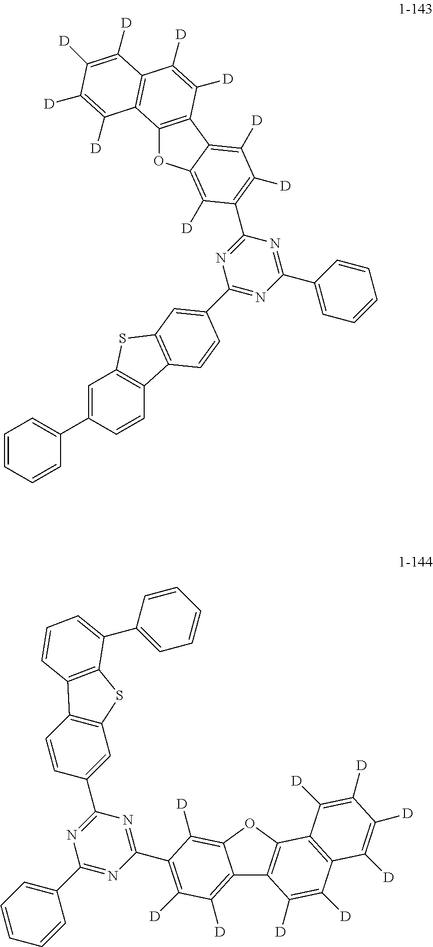 [SMILES: [2H]c1c([2H])c([2H])c2c(c1[2H])c([2H])c([2H])c1c2oc2c([2H])c(-c3nc(-c4ccccc4)nc(-c4ccc5c(c4)sc4cc(-c6ccccc6)ccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)sc4c(-c6ccccc6)cccc45)n3)c([2H])c([2H])c21]

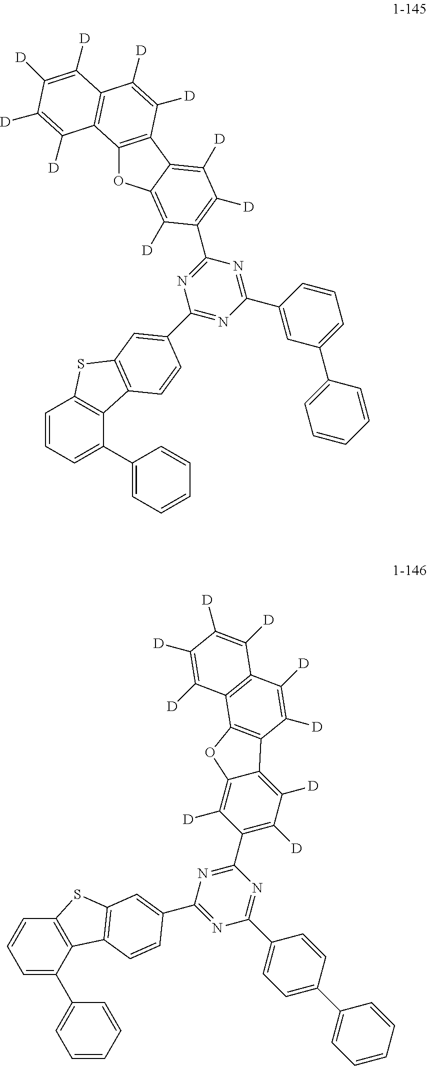 [SMILES: [2H]c1c([2H])c([2H])c2c(c1[2H])c([2H])c([2H])c1c2oc2c([2H])c(-c3nc(-c4cccc(-c5ccccc5)c4)nc(-c4ccc5c(c4)sc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4ccc(-c5ccccc5)cc4)nc(-c4ccc5c(c4)sc4cccc(-c6ccccc6)c45)n3)c([2H])c([2H])c21]

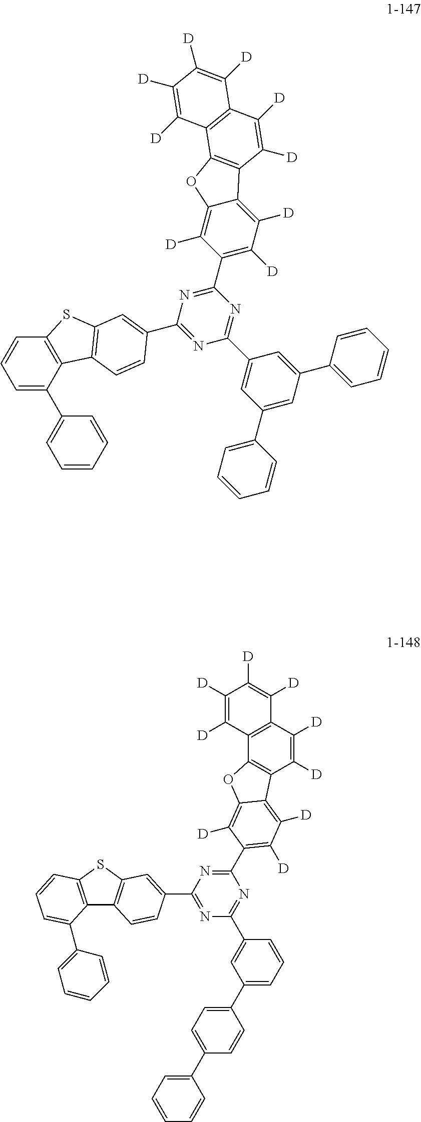 [SMILES: [2H]c1c([2H])c([2H])c2c(c1[2H])c([2H])c([2H])c1c2oc2c([2H])c(-c3nc(-c4cc(-c5ccccc5)cc(-c5ccccc5)c4)nc(-c4ccc5c(c4)sc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4cccc(-c5ccc(-c6ccccc6)cc5)c4)nc(-c4ccc5c(c4)sc4cccc(-c6ccccc6)c45)n3)c([2H])c([2H])c21]

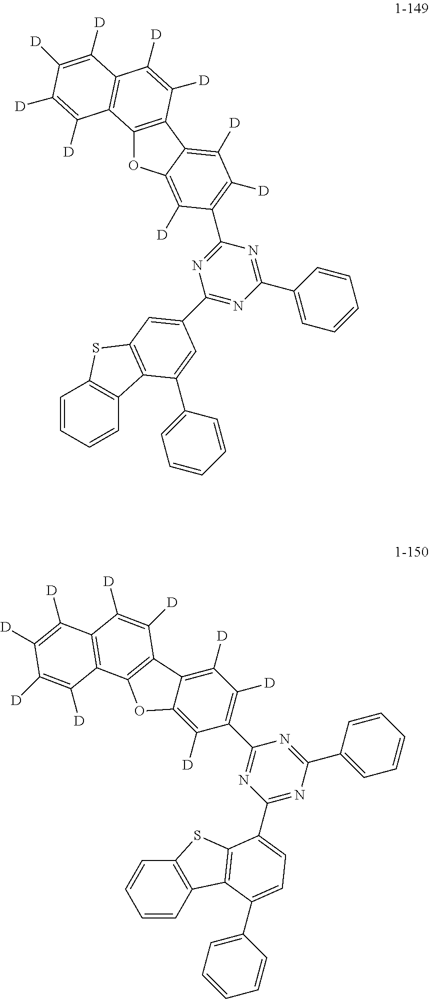 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)c5c(c4)sc4ccccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc(-c5ccccc5)c5c4sc4ccccc45)n3)c([2H])c([2H])c21]

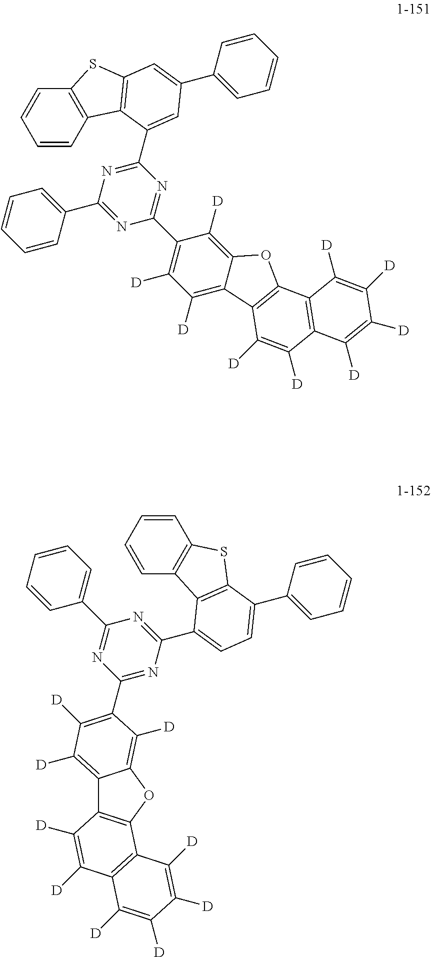 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)cc5sc6ccccc6c45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc(-c5ccccc5)c5sc6ccccc6c45)n3)c([2H])c([2H])c21]

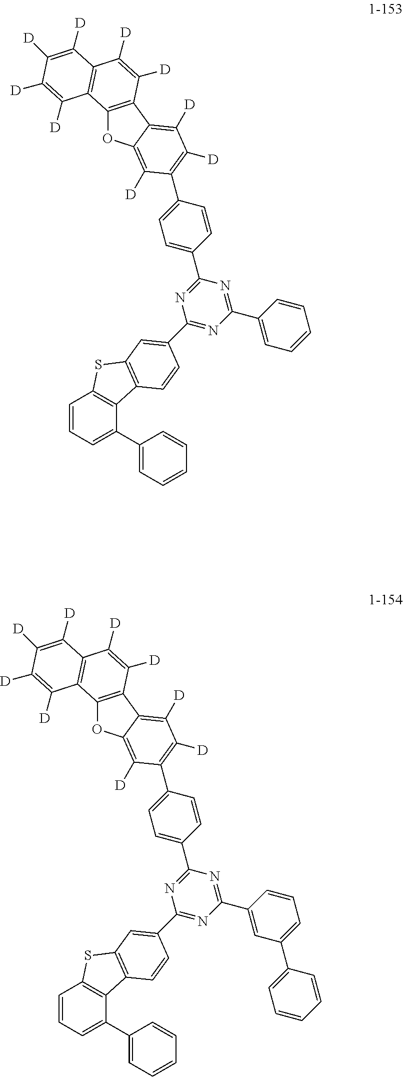 [SMILES: [2H]c1c([2H])c([2H])c2c(c1[2H])c([2H])c([2H])c1c2oc2c([2H])c(-c3ccc(-c4nc(-c5ccccc5)nc(-c5ccc6c(c5)sc5cccc(-c7ccccc7)c56)n4)cc3)c([2H])c([2H])c21 ; [2H]c1c([2H])c([2H])c2c(c1[2H])c([2H])c([2H])c1c2oc2c([2H])c(-c3ccc(-c4nc(-c5cccc(-c6ccccc6)c5)nc(-c5ccc6c(c5)sc5cccc(-c7ccccc7)c56)n4)cc3)c([2H])c([2H])c21]

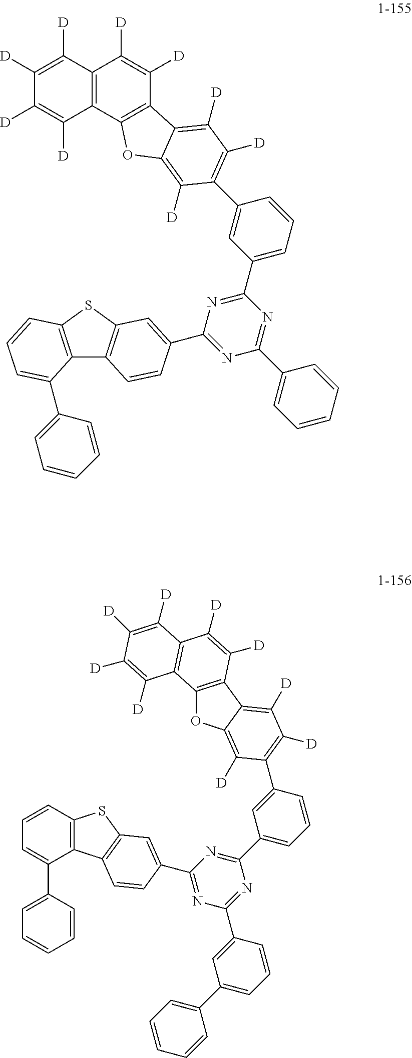 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)sc5cccc(-c7ccccc7)c56)n4)c3)c([2H])c([2H])c21 ; [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)sc5cccc(-c7ccccc7)c56)n4)c3)c([2H])c([2H])c21]

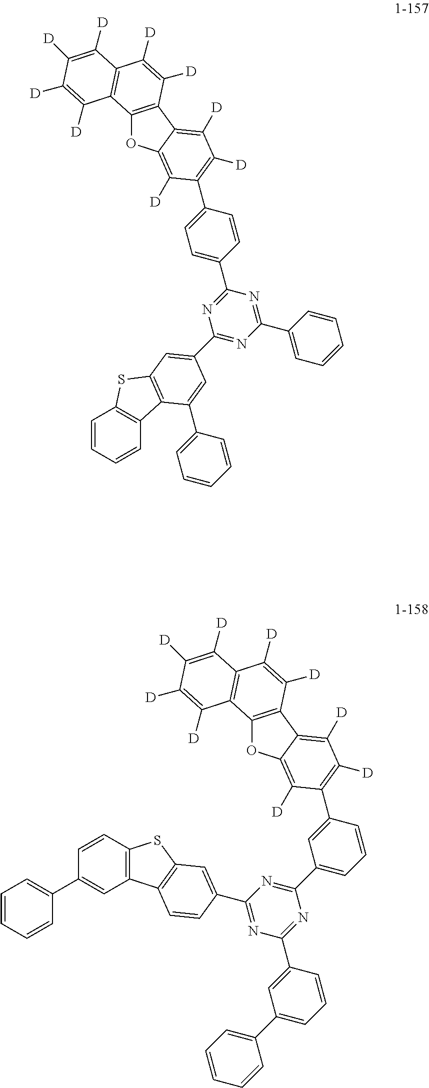 [SMILES: [2H]c1c([2H])c([2H])c2c(c1[2H])c([2H])c([2H])c1c2oc2c([2H])c(-c3ccc(-c4nc(-c5ccccc5)nc(-c5cc(-c6ccccc6)c6c(c5)sc5ccccc56)n4)cc3)c([2H])c([2H])c21 ; [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)sc5ccc(-c7ccccc7)cc56)n4)c3)c([2H])c([2H])c21]

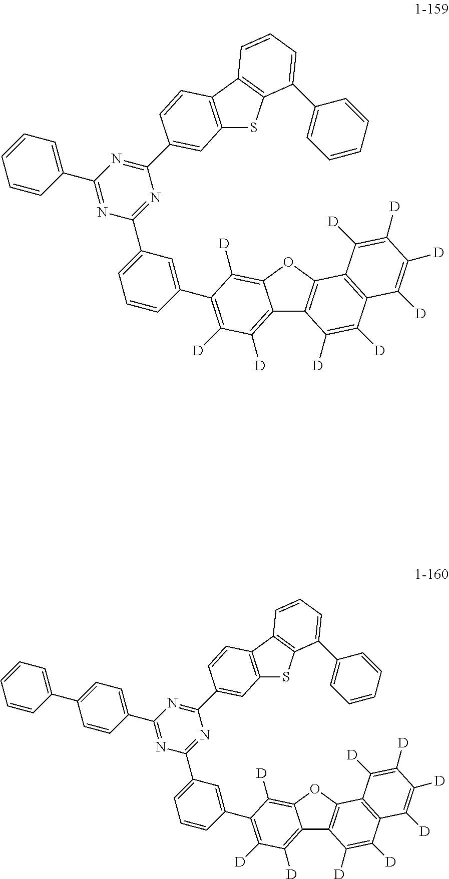 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)sc5c(-c7ccccc7)cccc56)n4)c3)c([2H])c([2H])c21 ; [2H]c1c([2H])c([2H])c2c(c1[2H])c([2H])c([2H])c1c2oc2c([2H])c(-c3cccc(-c4nc(-c5ccc(-c6ccccc6)cc5)nc(-c5ccc6c(c5)sc5c(-c7ccccc7)cccc56)n4)c3)c([2H])c([2H])c21]

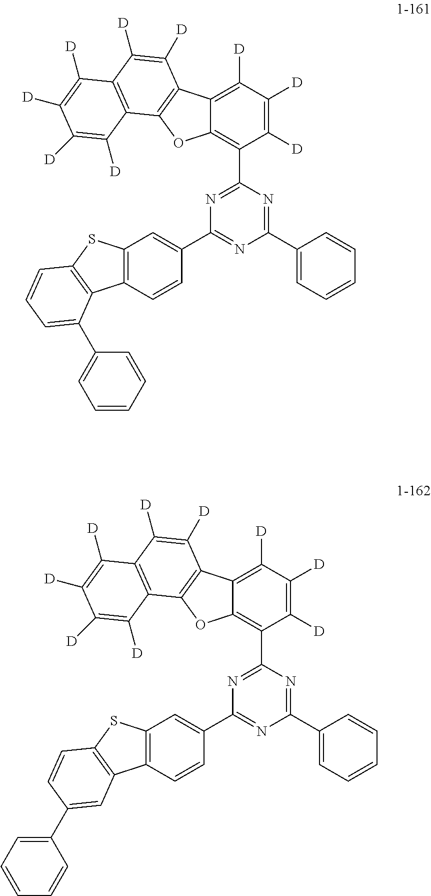 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2ccc(-c4ccccc4)cc23)n1]

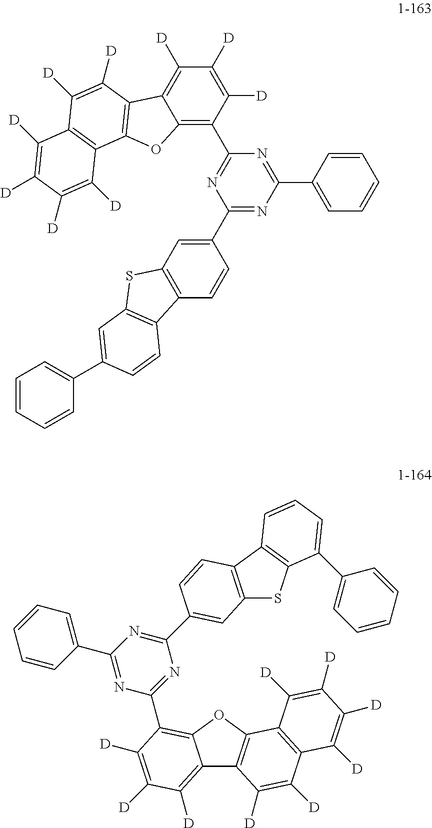 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2cc(-c4ccccc4)ccc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2c(-c4ccccc4)cccc23)n1]

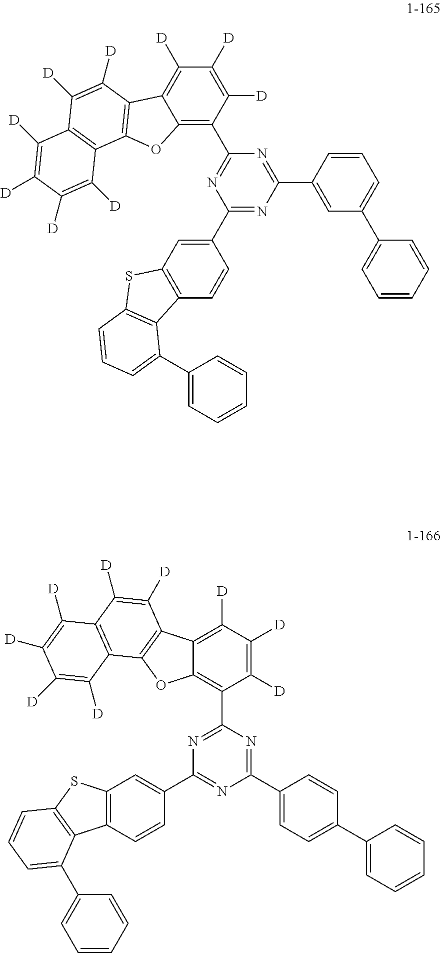 [SMILES: [2H]c1c([2H])c([2H])c2c(oc3c4c([2H])c([2H])c([2H])c([2H])c4c([2H])c([2H])c32)c1-c1nc(-c2cccc(-c3ccccc3)c2)nc(-c2ccc3c(c2)sc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccc(-c3ccccc3)cc2)nc(-c2ccc3c(c2)sc2cccc(-c4ccccc4)c23)n1]

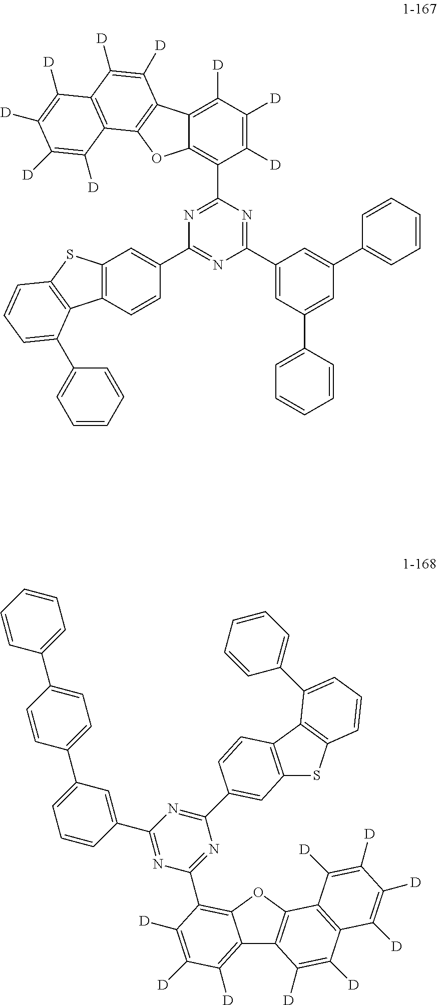 [SMILES: [2H]c1c([2H])c([2H])c2c(oc3c4c([2H])c([2H])c([2H])c([2H])c4c([2H])c([2H])c32)c1-c1nc(-c2cc(-c3ccccc3)cc(-c3ccccc3)c2)nc(-c2ccc3c(c2)sc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2cccc(-c3ccc(-c4ccccc4)cc3)c2)nc(-c2ccc3c(c2)sc2cccc(-c4ccccc4)c23)n1]

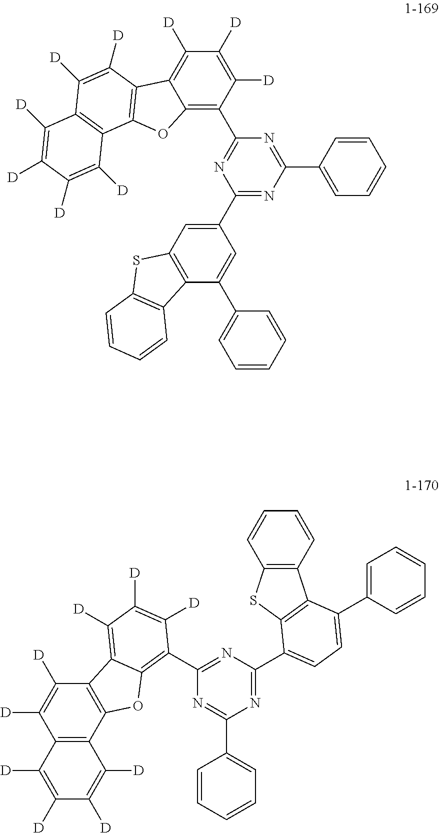 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)c3c(c2)sc2ccccc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3c2sc2ccccc23)n1]

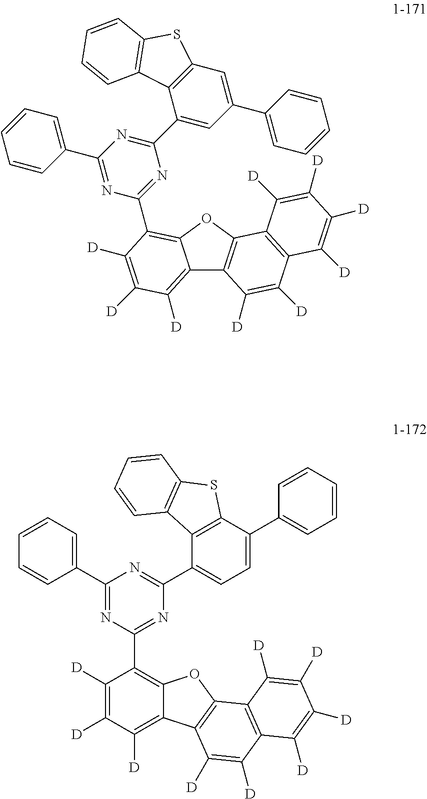 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)cc3sc4ccccc4c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3sc4ccccc4c23)n1]

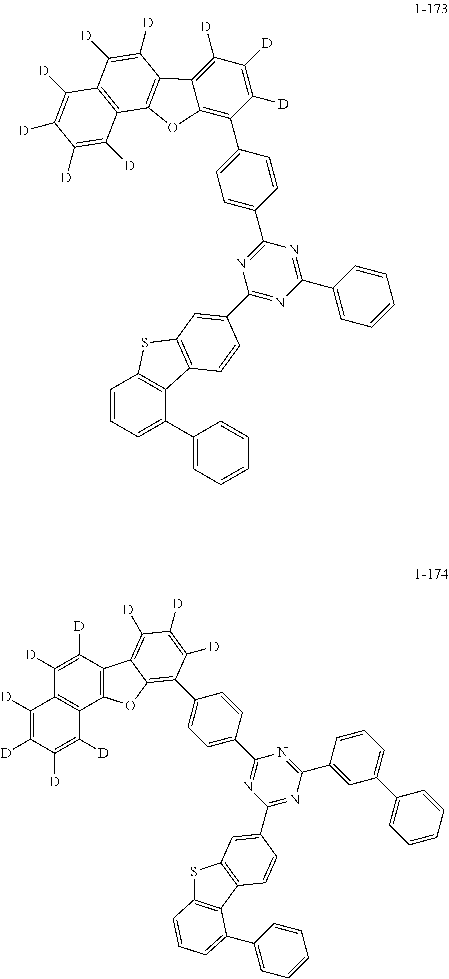 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3ccc4c(c3)sc3cccc(-c5ccccc5)c34)n2)cc1 ; [2H]c1c([2H])c([2H])c2c(oc3c4c([2H])c([2H])c([2H])c([2H])c4c([2H])c([2H])c32)c1-c1ccc(-c2nc(-c3cccc(-c4ccccc4)c3)nc(-c3ccc4c(c3)sc3cccc(-c5ccccc5)c34)n2)cc1]

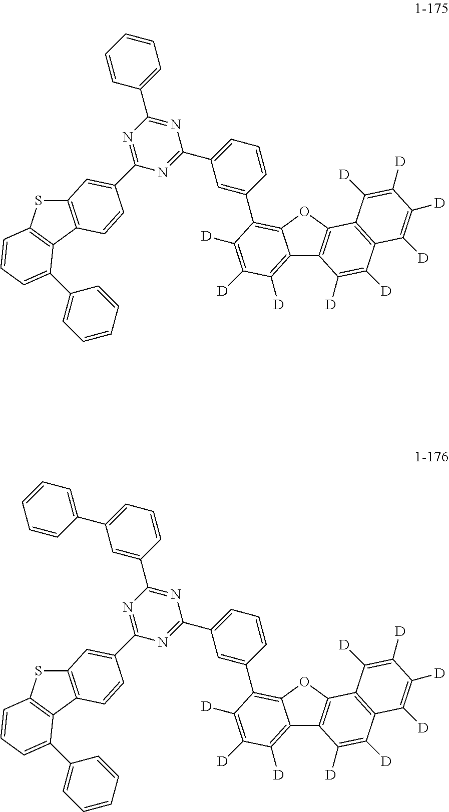 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)sc3cccc(-c5ccccc5)c34)n2)c1 ; [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3cccc(-c5ccccc5)c34)n2)c1]

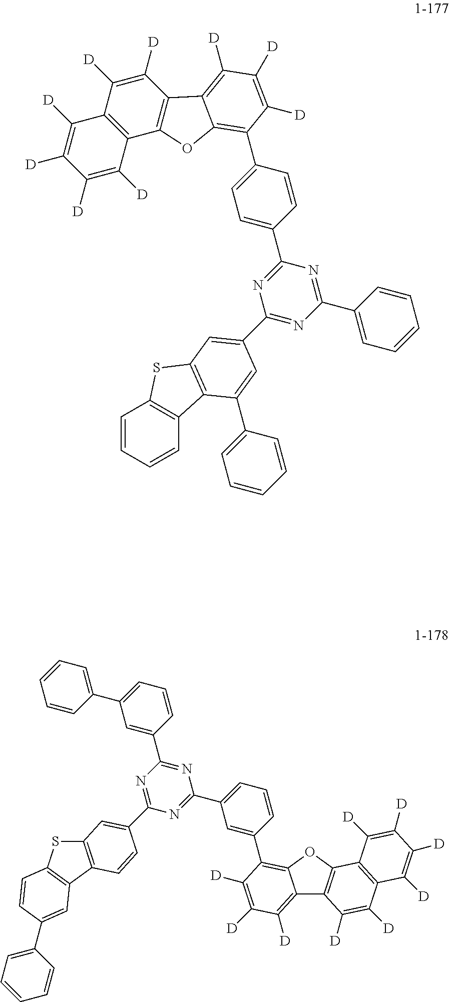 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3cc(-c4ccccc4)c4c(c3)sc3ccccc34)n2)cc1 ; [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3ccc(-c5ccccc5)cc34)n2)c1]

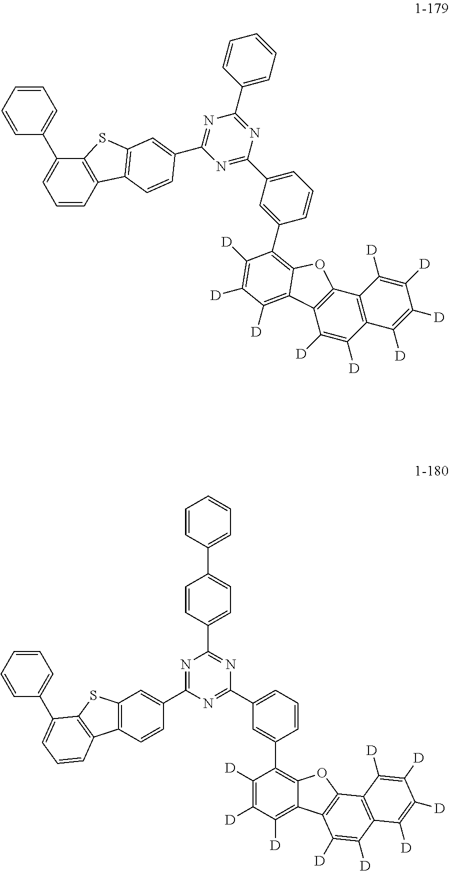 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)sc3c(-c5ccccc5)cccc34)n2)c1 ; [2H]c1c([2H])c([2H])c2c(oc3c4c([2H])c([2H])c([2H])c([2H])c4c([2H])c([2H])c32)c1-c1cccc(-c2nc(-c3ccc(-c4ccccc4)cc3)nc(-c3ccc4c(c3)sc3c(-c5ccccc5)cccc34)n2)c1]

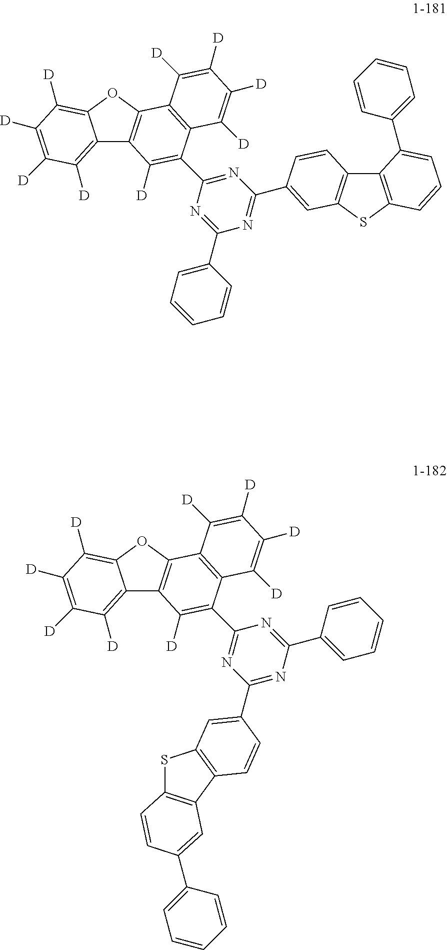 [SMILES: [2H]c1c([2H])c([2H])c2c(oc3c4c([2H])c([2H])c([2H])c([2H])c4c(-c4nc(-c5ccccc5)nc(-c5ccc6c(c5)sc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc6c(c5)sc5ccc(-c7ccccc7)cc56)n4)c([2H])c32)c1[2H]]

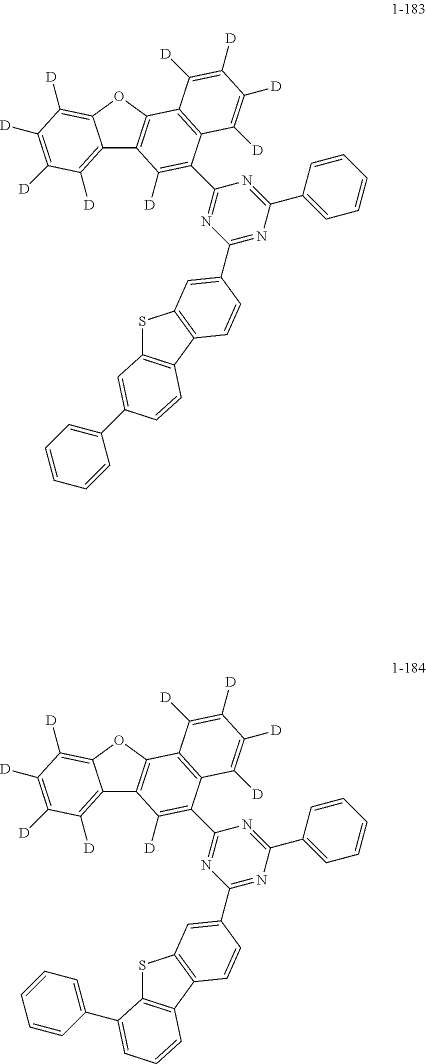 [SMILES: [2H]c1c([2H])c([2H])c2c(oc3c4c([2H])c([2H])c([2H])c([2H])c4c(-c4nc(-c5ccccc5)nc(-c5ccc6c(c5)sc5cc(-c7ccccc7)ccc56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc6c(c5)sc5c(-c7ccccc7)cccc56)n4)c([2H])c32)c1[2H]]

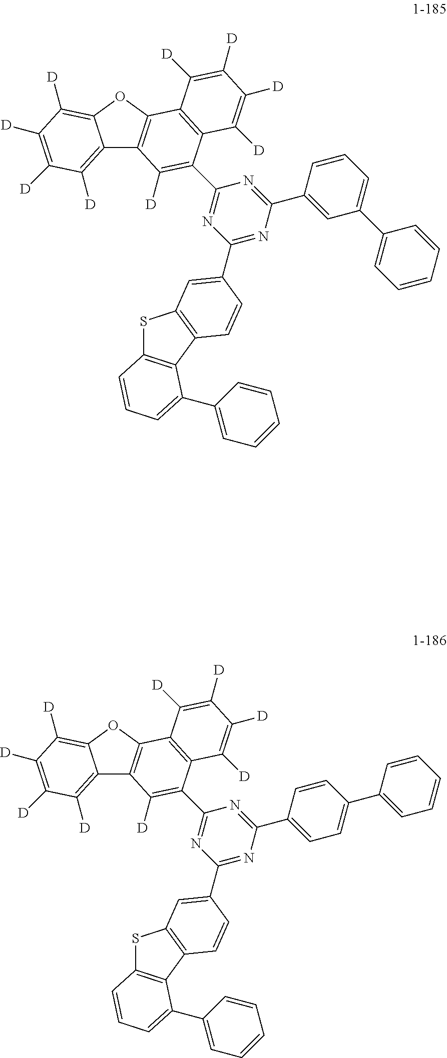 [SMILES: [2H]c1c([2H])c([2H])c2c(oc3c4c([2H])c([2H])c([2H])c([2H])c4c(-c4nc(-c5cccc(-c6ccccc6)c5)nc(-c5ccc6c(c5)sc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5ccc(-c6ccccc6)cc5)nc(-c5ccc6c(c5)sc5cccc(-c7ccccc7)c56)n4)c([2H])c32)c1[2H]]

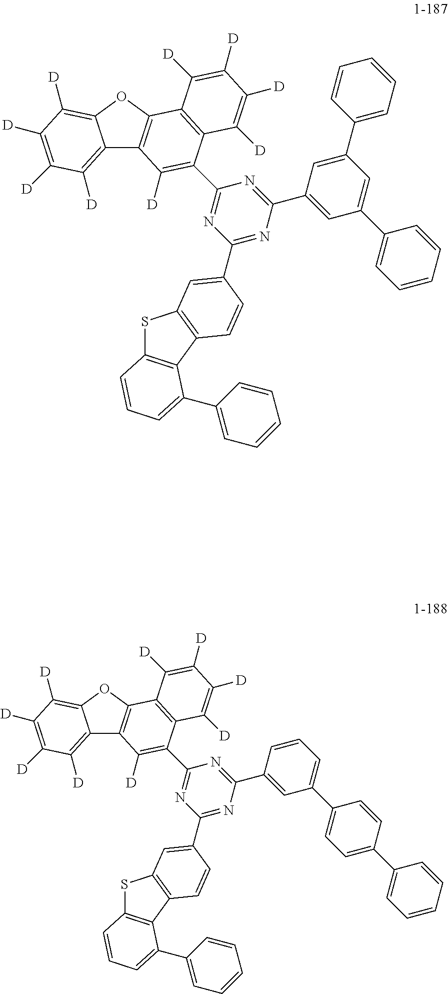 [SMILES: [2H]c1c([2H])c([2H])c2c(oc3c4c([2H])c([2H])c([2H])c([2H])c4c(-c4nc(-c5cc(-c6ccccc6)cc(-c6ccccc6)c5)nc(-c5ccc6c(c5)sc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5cccc(-c6ccc(-c7ccccc7)cc6)c5)nc(-c5ccc6c(c5)sc5cccc(-c7ccccc7)c56)n4)c([2H])c32)c1[2H]]

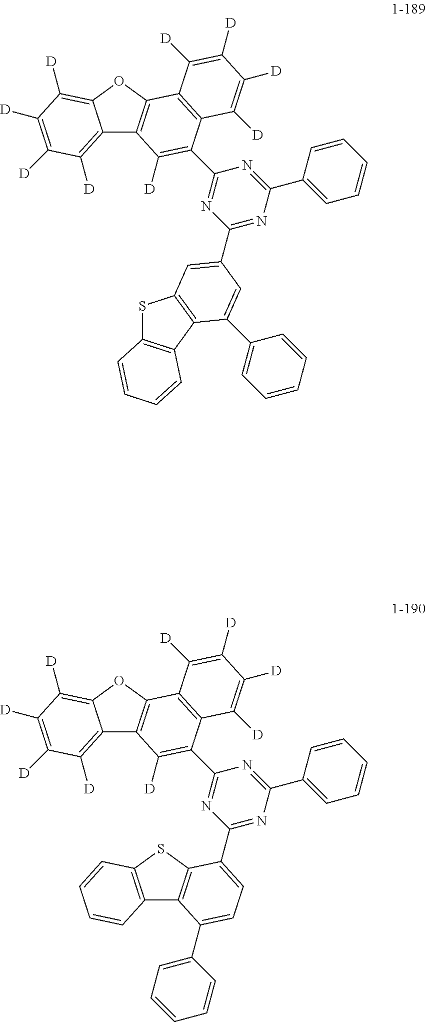 [SMILES: [2H]c1c([2H])c([2H])c2c(oc3c4c([2H])c([2H])c([2H])c([2H])c4c(-c4nc(-c5ccccc5)nc(-c5cc(-c6ccccc6)c6c(c5)sc5ccccc56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc(-c6ccccc6)c6c5sc5ccccc56)n4)c([2H])c32)c1[2H]]

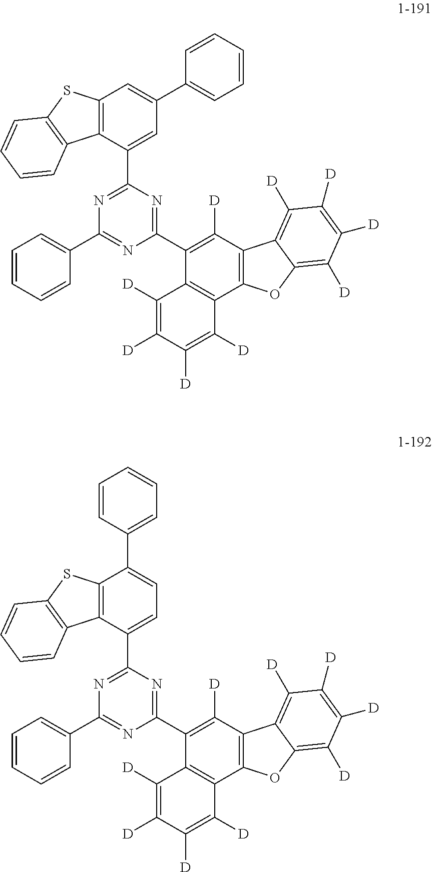 [SMILES: [2H]c1c([2H])c([2H])c2c(oc3c4c([2H])c([2H])c([2H])c([2H])c4c(-c4nc(-c5ccccc5)nc(-c5cc(-c6ccccc6)cc6sc7ccccc7c56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc(-c6ccccc6)c6sc7ccccc7c56)n4)c([2H])c32)c1[2H]]

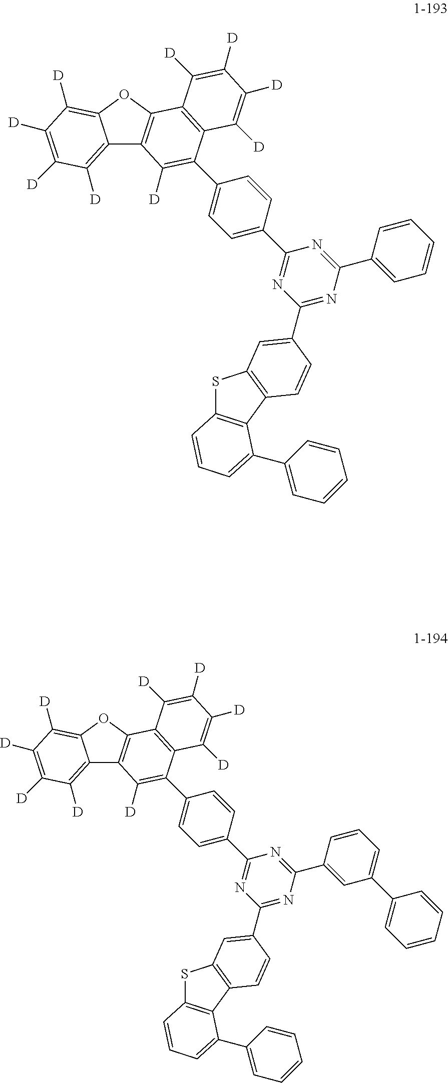 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)sc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)sc6cccc(-c8ccccc8)c67)n5)cc4)c([2H])c32)c1[2H]]

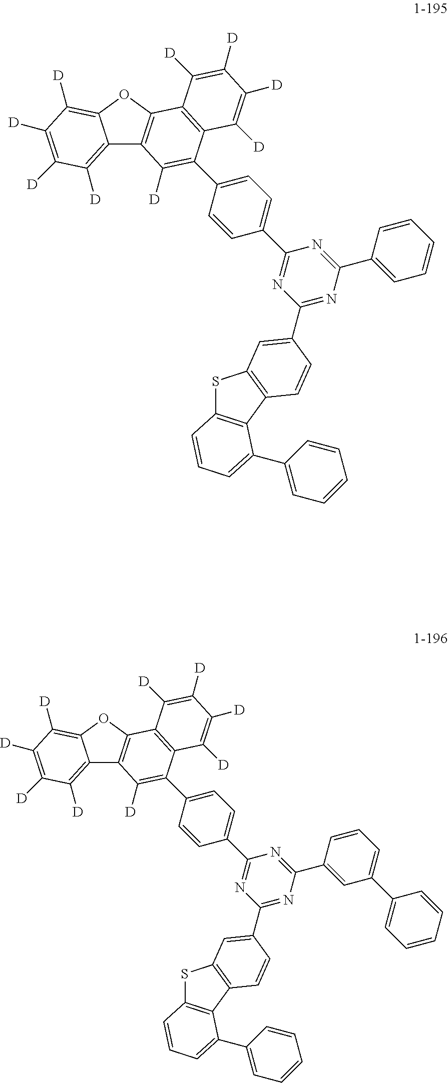 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)sc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)sc6cccc(-c8ccccc8)c67)n5)cc4)c([2H])c32)c1[2H]]

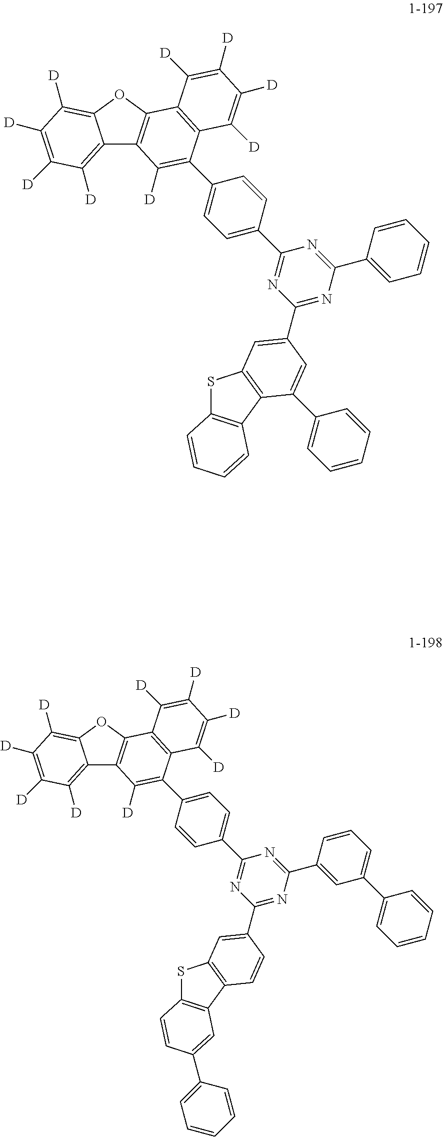 [SMILES: [2H]c1c([2H])c([2H])c2c(oc3c4c([2H])c([2H])c([2H])c([2H])c4c(-c4ccc(-c5nc(-c6ccccc6)nc(-c6cc(-c7ccccc7)c7c(c6)sc6ccccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)sc6ccc(-c8ccccc8)cc67)n5)cc4)c([2H])c32)c1[2H]]

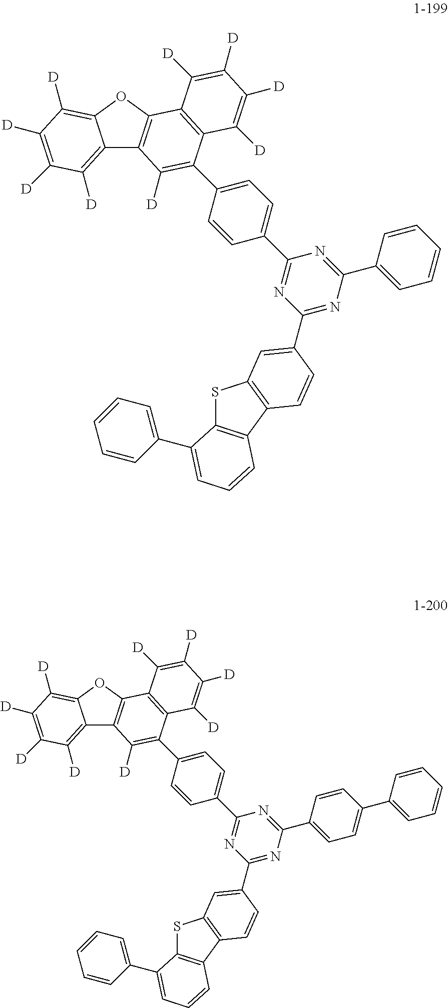 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)sc6c(-c8ccccc8)cccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6ccc(-c7ccccc7)cc6)nc(-c6ccc7c(c6)sc6c(-c8ccccc8)cccc67)n5)cc4)c([2H])c32)c1[2H]]

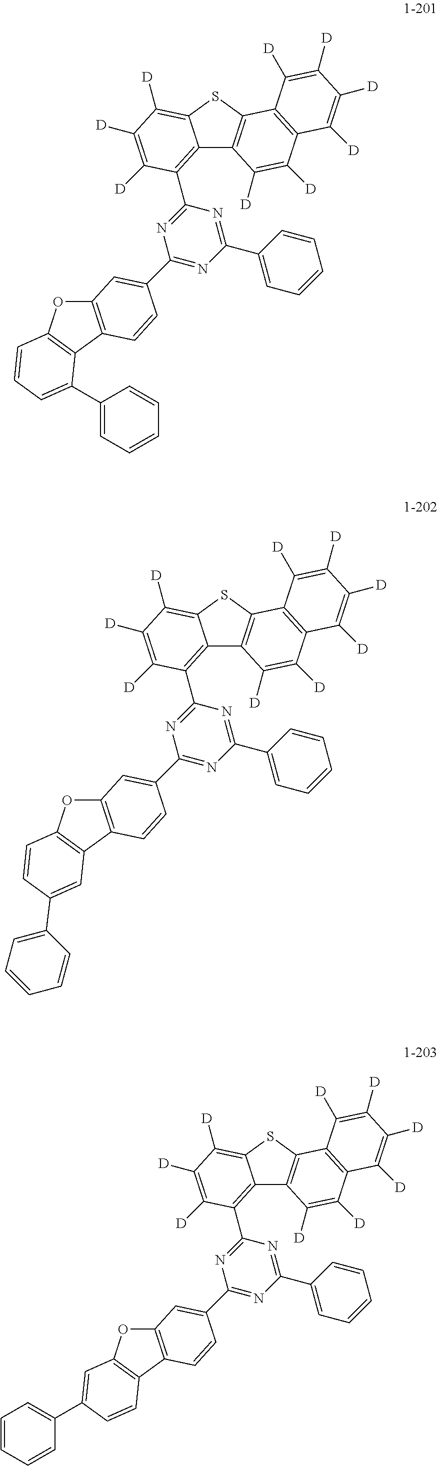 [SMILES: [2H]c1c([2H])c(-c2nc(-c3ccccc3)nc(-c3ccc4c(c3)oc3cccc(-c5ccccc5)c34)n2)c2c(sc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3ccc(-c5ccccc5)cc34)n2)c2c(sc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3cc(-c5ccccc5)ccc34)n2)c2c(sc3c4c([2H])c([2H])c([2H])c([2H])c4c([2H])c([2H])c32)c1[2H]]

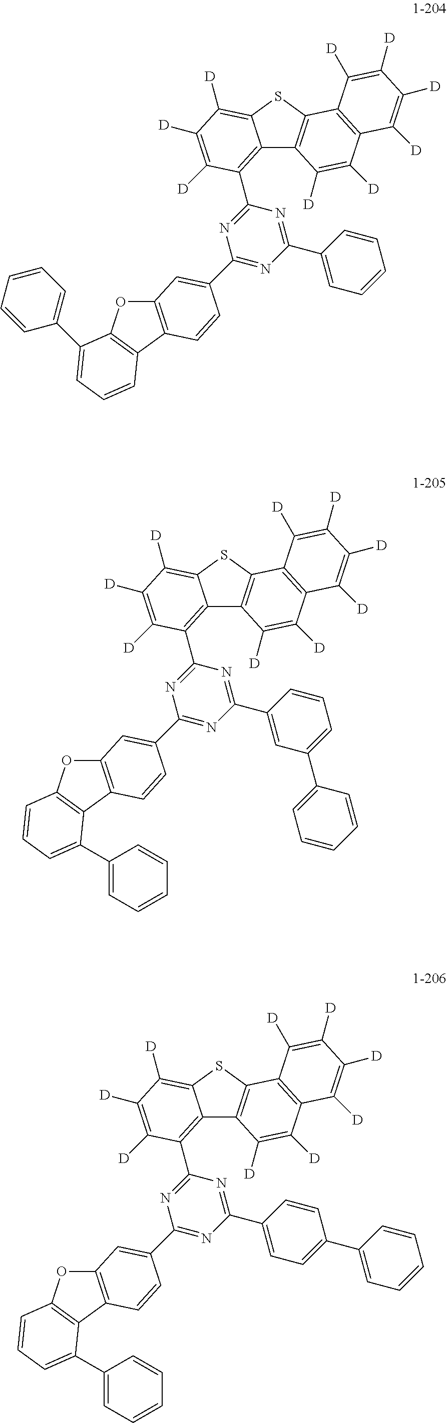 [SMILES: [2H]c1c([2H])c(-c2nc(-c3ccccc3)nc(-c3ccc4c(c3)oc3c(-c5ccccc5)cccc34)n2)c2c(sc3c4c([2H])c([2H])c([2H])c([2H])c4c([2H])c([2H])c32)c1[2H] ; [2H]c1c([2H])c(-c2nc(-c3cccc(-c4ccccc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H] ; [2H]c1c([2H])c(-c2nc(-c3ccc(-c4ccccc4)cc3)nc(-c3ccc4c(c3)oc3cccc(-c5ccccc5)c34)n2)c2c(sc3c4c([2H])c([2H])c([2H])c([2H])c4c([2H])c([2H])c32)c1[2H]]

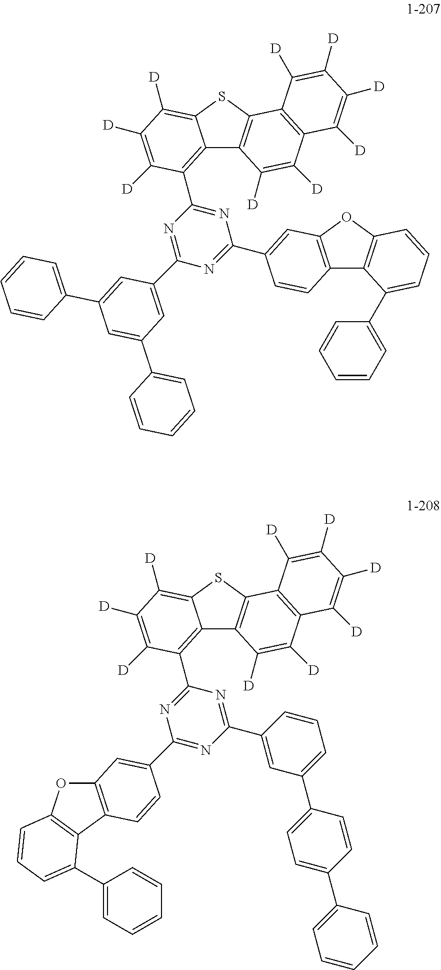 [SMILES: [2H]c1c([2H])c(-c2nc(-c3cc(-c4ccccc4)cc(-c4ccccc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H] ; [2H]c1c([2H])c(-c2nc(-c3cccc(-c4ccc(-c5ccccc5)cc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H]]

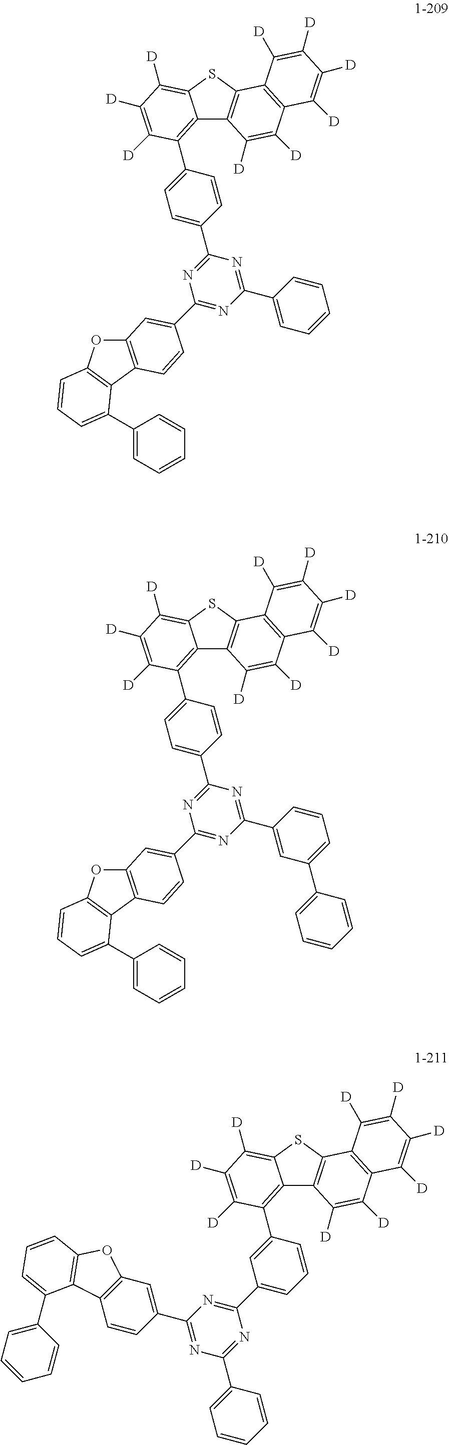 [SMILES: [2H]c1c([2H])c(-c2ccc(-c3nc(-c4ccccc4)nc(-c4ccc5c(c4)oc4cccc(-c6ccccc6)c45)n3)cc2)c2c(sc3c4c([2H])c([2H])c([2H])c([2H])c4c([2H])c([2H])c32)c1[2H] ; [2H]c1c([2H])c(-c2ccc(-c3nc(-c4cccc(-c5ccccc5)c4)nc(-c4ccc5c(c4)oc4cccc(-c6ccccc6)c45)n3)cc2)c2c(sc3c4c([2H])c([2H])c([2H])c([2H])c4c([2H])c([2H])c32)c1[2H] ; [2H]c1c([2H])c(-c2cccc(-c3nc(-c4ccccc4)nc(-c4ccc5c(c4)oc4cccc(-c6ccccc6)c45)n3)c2)c2c(sc3c4c([2H])c([2H])c([2H])c([2H])c4c([2H])c([2H])c32)c1[2H]]

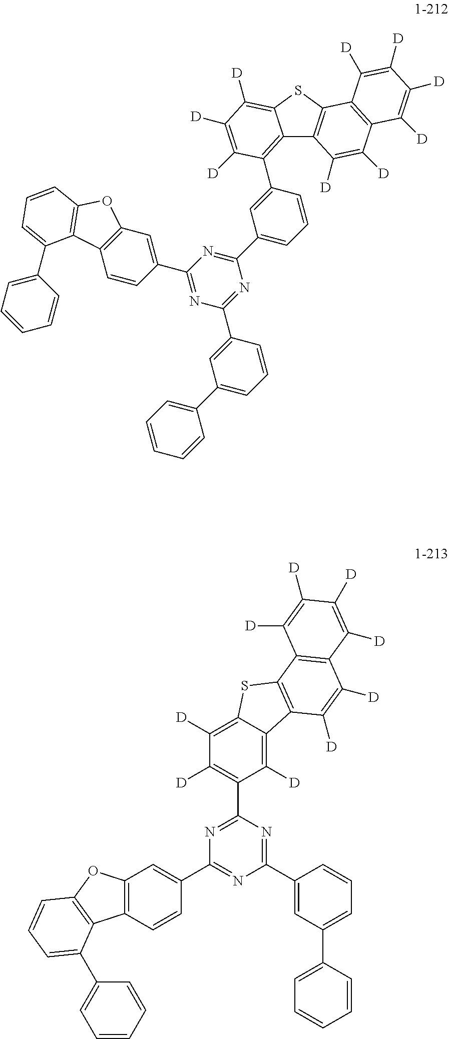 [SMILES: [2H]c1c([2H])c(-c2cccc(-c3nc(-c4cccc(-c5ccccc5)c4)nc(-c4ccc5c(c4)oc4cccc(-c6ccccc6)c45)n3)c2)c2c(sc3c4c([2H])c([2H])c([2H])c([2H])c4c([2H])c([2H])c32)c1[2H] ; [2H]c1c(-c2nc(-c3cccc(-c4ccccc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H]]

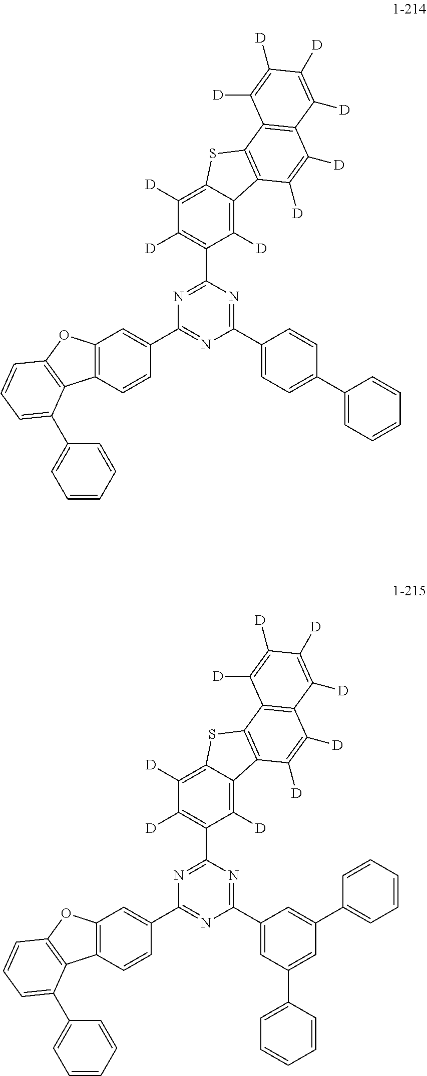 [SMILES: [2H]c1c(-c2nc(-c3ccc(-c4ccccc4)cc3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H] ; [2H]c1c(-c2nc(-c3cc(-c4ccccc4)cc(-c4ccccc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H]]

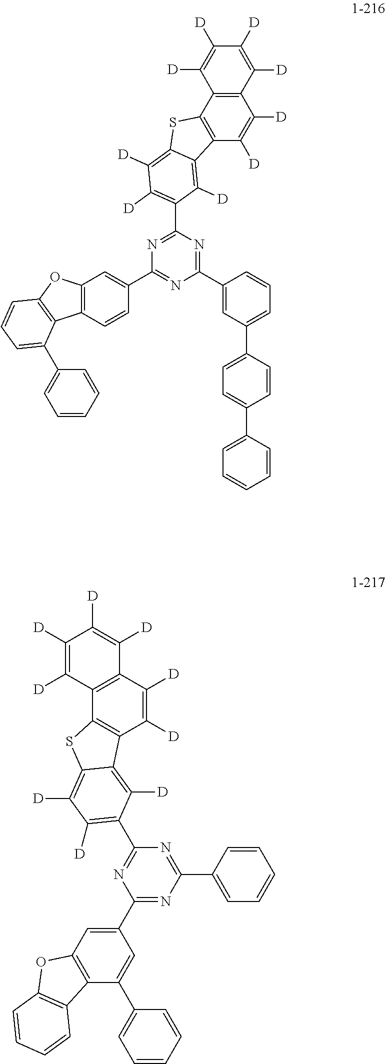 [SMILES: [2H]c1c(-c2nc(-c3cccc(-c4ccc(-c5ccccc5)cc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H] ; [2H]c1c(-c2nc(-c3ccccc3)nc(-c3cc(-c4ccccc4)c4c(c3)oc3ccccc34)n2)c([2H])c2c(sc3c4c([2H])c([2H])c([2H])c([2H])c4c([2H])c([2H])c32)c1[2H]]

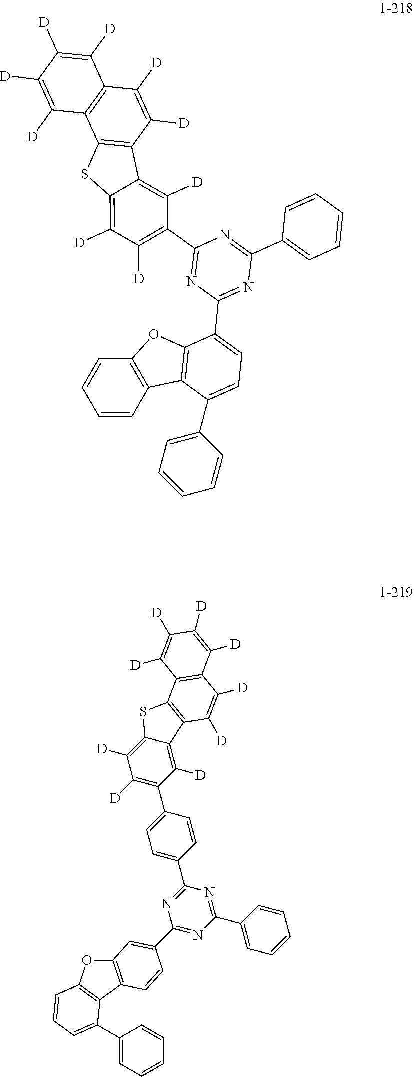 [SMILES: [2H]c1c(-c2nc(-c3ccccc3)nc(-c3ccc(-c4ccccc4)c4c3oc3ccccc34)n2)c([2H])c2c(sc3c4c([2H])c([2H])c([2H])c([2H])c4c([2H])c([2H])c32)c1[2H] ; [2H]c1c(-c2ccc(-c3nc(-c4ccccc4)nc(-c4ccc5c(c4)oc4cccc(-c6ccccc6)c45)n3)cc2)c([2H])c2c(sc3c4c([2H])c([2H])c([2H])c([2H])c4c([2H])c([2H])c32)c1[2H]]

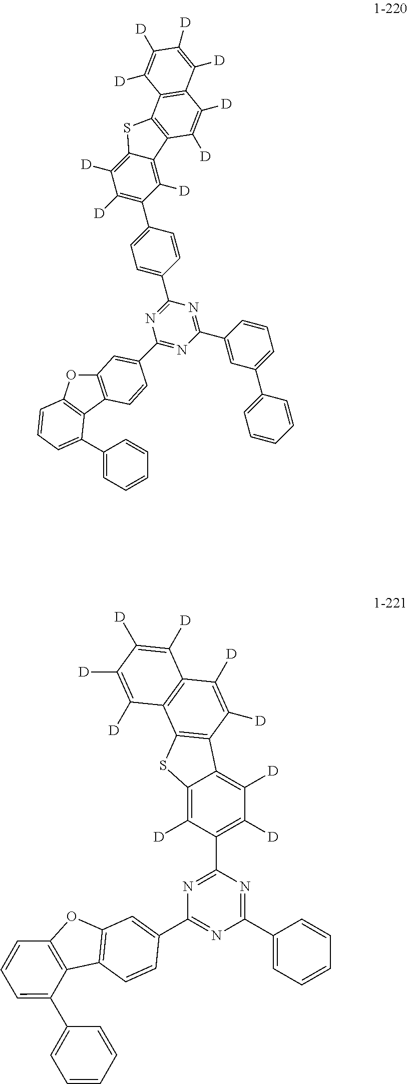 [SMILES: [2H]c1c(-c2ccc(-c3nc(-c4cccc(-c5ccccc5)c4)nc(-c4ccc5c(c4)oc4cccc(-c6ccccc6)c45)n3)cc2)c([2H])c2c(sc3c4c([2H])c([2H])c([2H])c([2H])c4c([2H])c([2H])c32)c1[2H] ; [2H]c1c([2H])c([2H])c2c(c1[2H])c([2H])c([2H])c1c2sc2c([2H])c(-c3nc(-c4ccccc4)nc(-c4ccc5c(c4)oc4cccc(-c6ccccc6)c45)n3)c([2H])c([2H])c21]

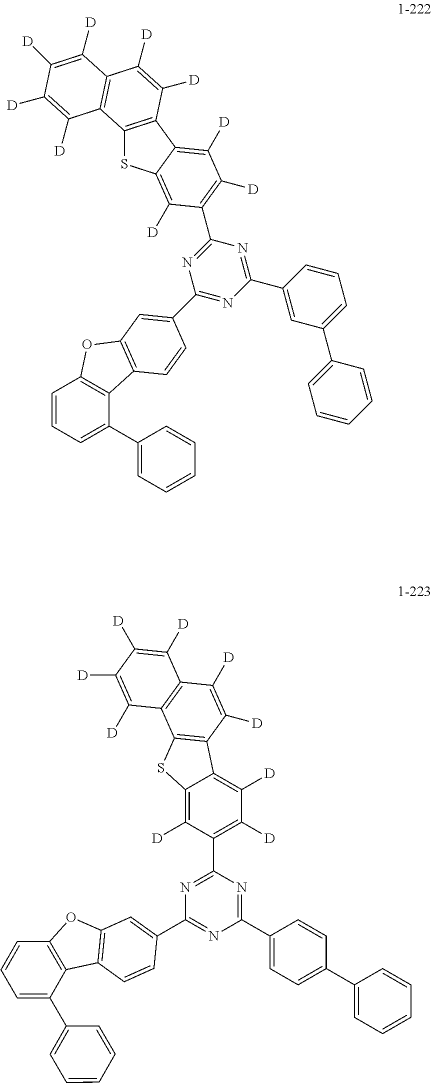 [SMILES: [2H]c1c([2H])c([2H])c2c(c1[2H])c([2H])c([2H])c1c2sc2c([2H])c(-c3nc(-c4cccc(-c5ccccc5)c4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2sc2c([2H])c(-c3nc(-c4ccc(-c5ccccc5)cc4)nc(-c4ccc5c(c4)oc4cccc(-c6ccccc6)c45)n3)c([2H])c([2H])c21]

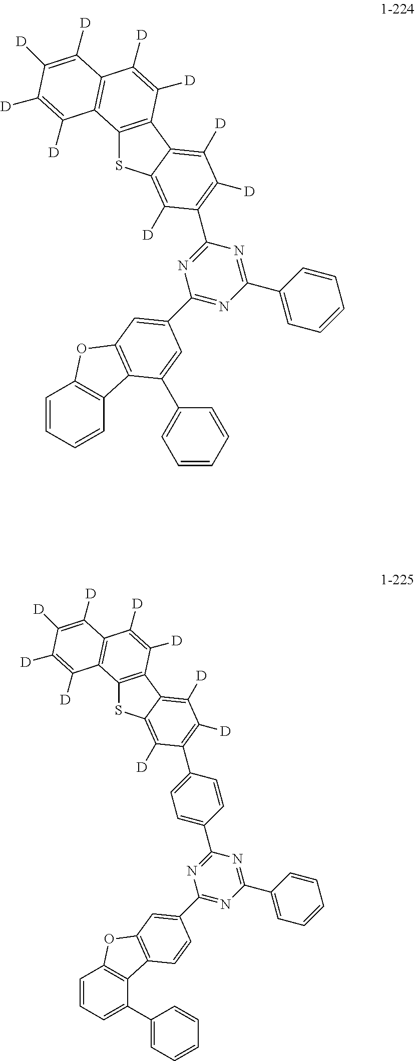 [SMILES: [2H]c1c([2H])c([2H])c2c(c1[2H])c([2H])c([2H])c1c2sc2c([2H])c(-c3nc(-c4ccccc4)nc(-c4cc(-c5ccccc5)c5c(c4)oc4ccccc45)n3)c([2H])c([2H])c21 ; [2H]c1c([2H])c([2H])c2c(c1[2H])c([2H])c([2H])c1c2sc2c([2H])c(-c3ccc(-c4nc(-c5ccccc5)nc(-c5ccc6c(c5)oc5cccc(-c7ccccc7)c56)n4)cc3)c([2H])c([2H])c21]

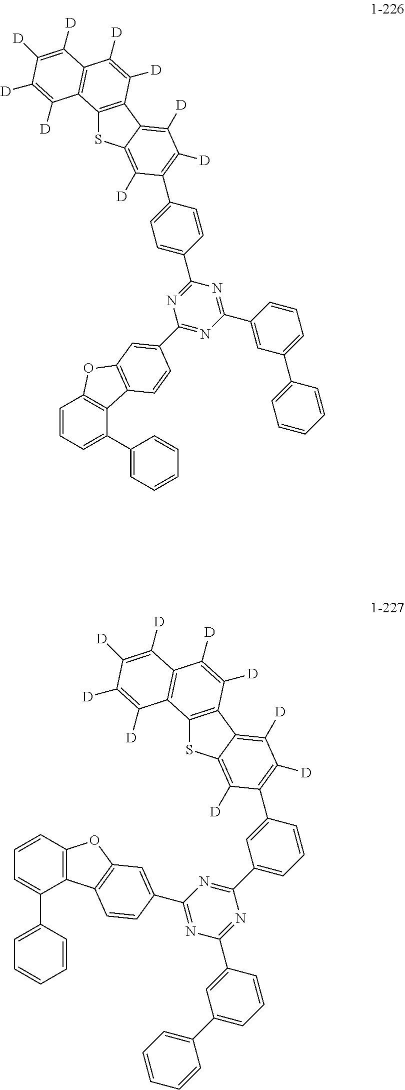 [SMILES: [2H]c1c([2H])c([2H])c2c(c1[2H])c([2H])c([2H])c1c2sc2c([2H])c(-c3ccc(-c4nc(-c5cccc(-c6ccccc6)c5)nc(-c5ccc6c(c5)oc5cccc(-c7ccccc7)c56)n4)cc3)c([2H])c([2H])c21 ; [2H]c1c([2H])c([2H])c2c(c1[2H])c([2H])c([2H])c1c2sc2c([2H])c(-c3cccc(-c4nc(-c5cccc(-c6ccccc6)c5)nc(-c5ccc6c(c5)oc5cccc(-c7ccccc7)c56)n4)c3)c([2H])c([2H])c21]

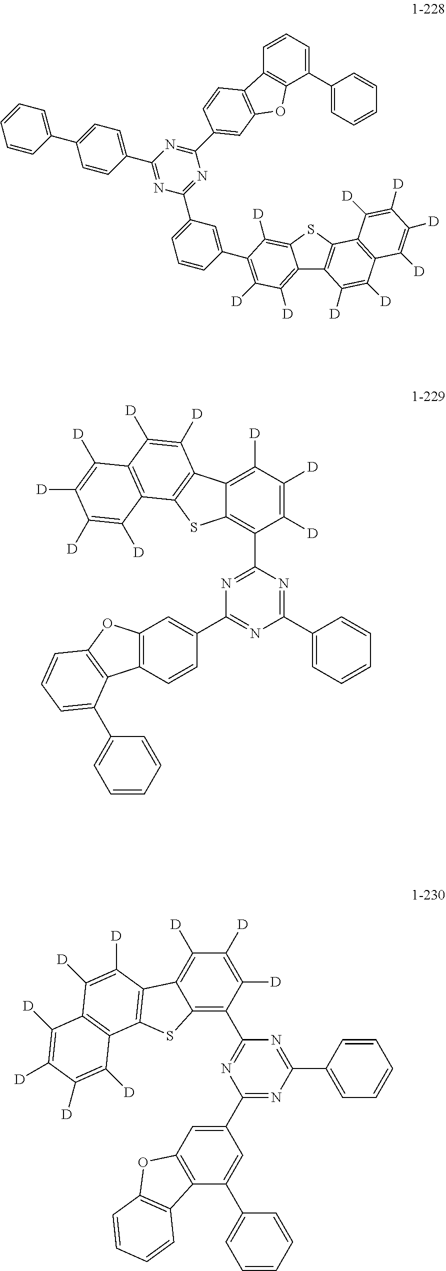 [SMILES: [2H]c1c([2H])c([2H])c2c(c1[2H])c([2H])c([2H])c1c2sc2c([2H])c(-c3cccc(-c4nc(-c5ccc(-c6ccccc6)cc5)nc(-c5ccc6c(c5)oc5c(-c7ccccc7)cccc56)n4)c3)c([2H])c([2H])c21 ; [2H]c1c([2H])c([2H])c2c(sc3c4c([2H])c([2H])c([2H])c([2H])c4c([2H])c([2H])c32)c1-c1nc(-c2ccccc2)nc(-c2ccc3c(c2)oc2cccc(-c4ccccc4)c23)n1 ; [2H]c1c([2H])c([2H])c2c(sc3c4c([2H])c([2H])c([2H])c([2H])c4c([2H])c([2H])c32)c1-c1nc(-c2ccccc2)nc(-c2cc(-c3ccccc3)c3c(c2)oc2ccccc23)n1]

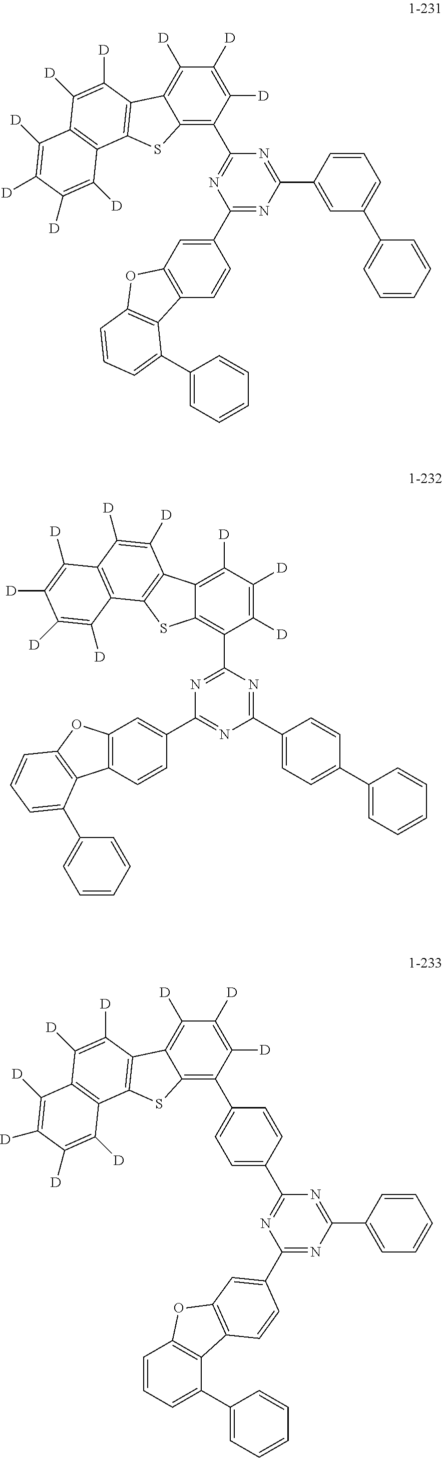 [SMILES: [2H]c1c([2H])c([2H])c2c(sc3c4c([2H])c([2H])c([2H])c([2H])c4c([2H])c([2H])c32)c1-c1nc(-c2cccc(-c3ccccc3)c2)nc(-c2ccc3c(c2)oc2cccc(-c4ccccc4)c23)n1 ; [2H]c1c([2H])c([2H])c2c(sc3c4c([2H])c([2H])c([2H])c([2H])c4c([2H])c([2H])c32)c1-c1nc(-c2ccc(-c3ccccc3)cc2)nc(-c2ccc3c(c2)oc2cccc(-c4ccccc4)c23)n1 ; [2H]c1c([2H])c([2H])c2c(sc3c4c([2H])c([2H])c([2H])c([2H])c4c([2H])c([2H])c32)c1-c1ccc(-c2nc(-c3ccccc3)nc(-c3ccc4c(c3)oc3cccc(-c5ccccc5)c34)n2)cc1]

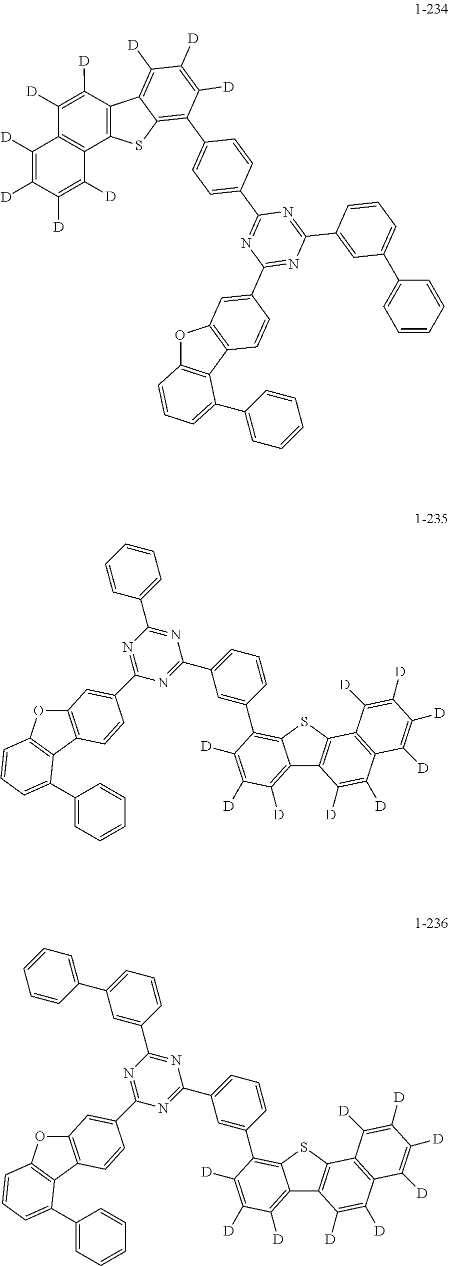 [SMILES: [2H]c1c([2H])c([2H])c2c(sc3c4c([2H])c([2H])c([2H])c([2H])c4c([2H])c([2H])c32)c1-c1ccc(-c2nc(-c3cccc(-c4ccccc4)c3)nc(-c3ccc4c(c3)oc3cccc(-c5ccccc5)c34)n2)cc1 ; [2H]c1c([2H])c([2H])c2c(sc3c4c([2H])c([2H])c([2H])c([2H])c4c([2H])c([2H])c32)c1-c1cccc(-c2nc(-c3ccccc3)nc(-c3ccc4c(c3)oc3cccc(-c5ccccc5)c34)n2)c1 ; [2H]c1c([2H])c([2H])c2c(sc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3cccc(-c5ccccc5)c34)n2)c1]

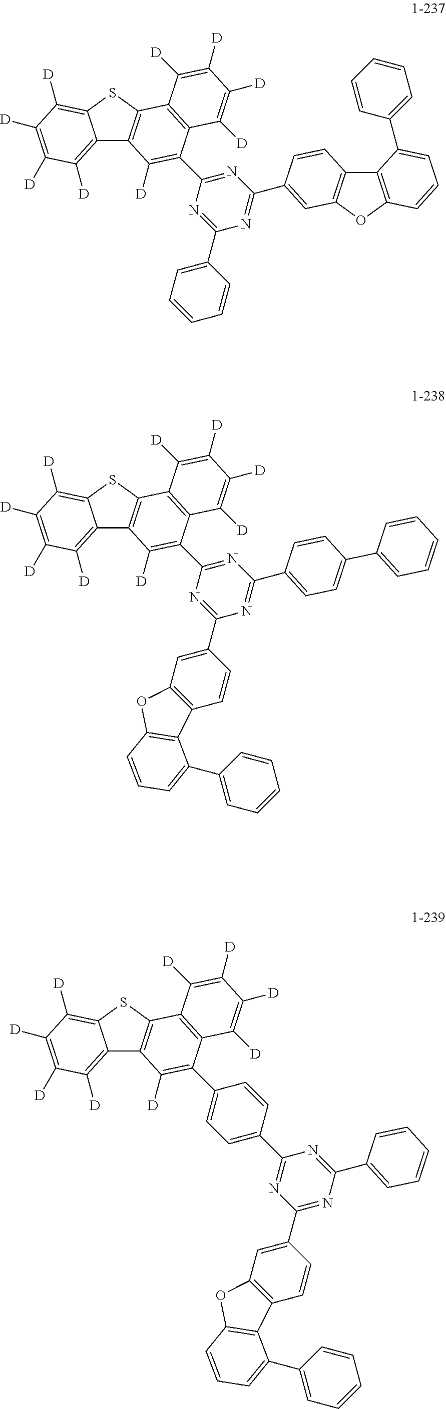 [SMILES: [2H]c1c([2H])c([2H])c2c(sc3c4c([2H])c([2H])c([2H])c([2H])c4c(-c4nc(-c5ccccc5)nc(-c5ccc6c(c5)oc5cccc(-c7ccccc7)c56)n4)c([2H])c32)c1[2H] ; [2H]c1c([2H])c([2H])c2c(sc3c4c([2H])c([2H])c([2H])c([2H])c4c(-c4nc(-c5ccc(-c6ccccc6)cc5)nc(-c5ccc6c(c5)oc5cccc(-c7ccccc7)c56)n4)c([2H])c32)c1[2H] ; [2H]c1c([2H])c([2H])c2c(sc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H]]

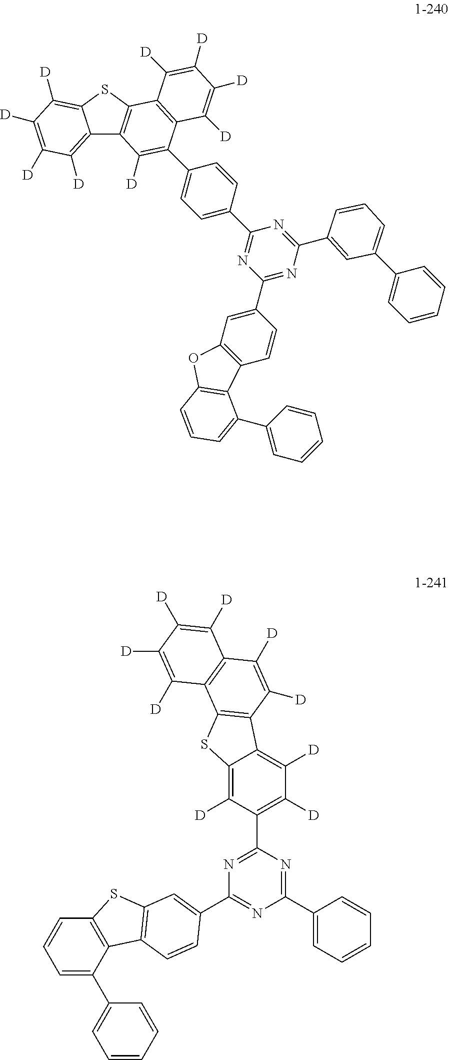 [SMILES: [2H]c1c([2H])c([2H])c2c(sc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H] ; [2H]c1c([2H])c([2H])c2c(c1[2H])c([2H])c([2H])c1c2sc2c([2H])c(-c3nc(-c4ccccc4)nc(-c4ccc5c(c4)sc4cccc(-c6ccccc6)c45)n3)c([2H])c([2H])c21]

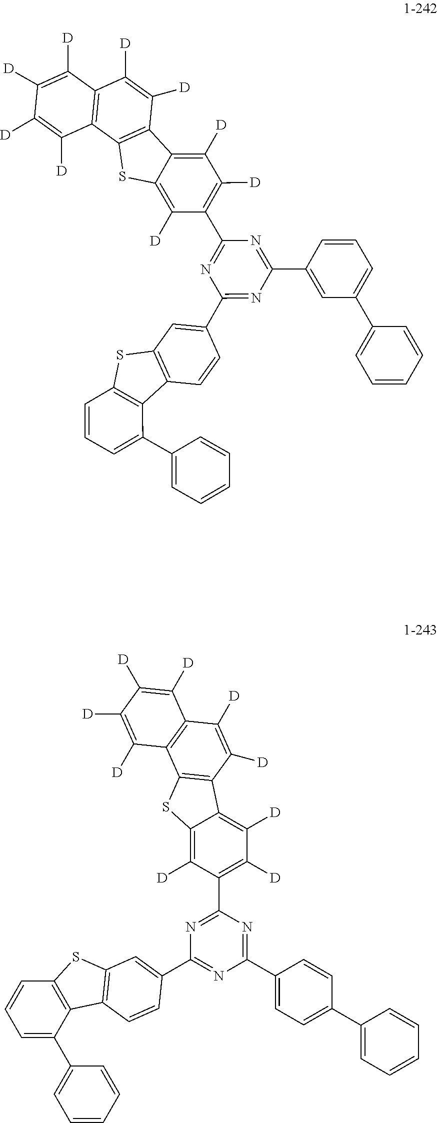 [SMILES: [2H]c1c([2H])c([2H])c2c(c1[2H])c([2H])c([2H])c1c2sc2c([2H])c(-c3nc(-c4cccc(-c5ccccc5)c4)nc(-c4ccc5c(c4)sc4cccc(-c6ccccc6)c45)n3)c([2H])c([2H])c21 ; [2H]c1c([2H])c([2H])c2c(c1[2H])c([2H])c([2H])c1c2sc2c([2H])c(-c3nc(-c4ccc(-c5ccccc5)cc4)nc(-c4ccc5c(c4)sc4cccc(-c6ccccc6)c45)n3)c([2H])c([2H])c21]

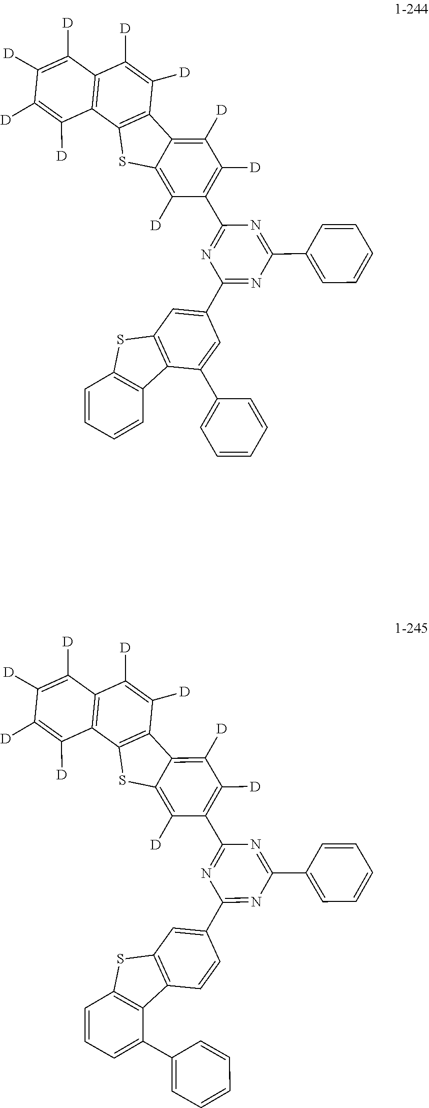 [SMILES: [2H]c1c([2H])c([2H])c2c(c1[2H])c([2H])c([2H])c1c2sc2c([2H])c(-c3nc(-c4ccccc4)nc(-c4cc(-c5ccccc5)c5c(c4)sc4ccccc45)n3)c([2H])c([2H])c21 ; [2H]c1c([2H])c([2H])c2c(c1[2H])c([2H])c([2H])c1c2sc2c([2H])c(-c3nc(-c4ccccc4)nc(-c4ccc5c(c4)sc4cccc(-c6ccccc6)c45)n3)c([2H])c([2H])c21]

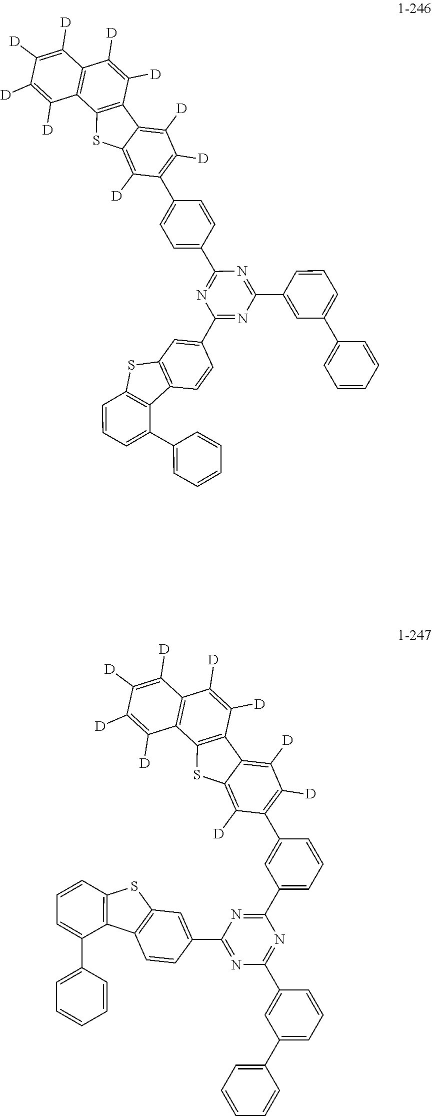 [SMILES: [2H]c1c([2H])c([2H])c2c(c1[2H])c([2H])c([2H])c1c2sc2c([2H])c(-c3ccc(-c4nc(-c5cccc(-c6ccccc6)c5)nc(-c5ccc6c(c5)sc5cccc(-c7ccccc7)c56)n4)cc3)c([2H])c([2H])c21 ; [2H]c1c([2H])c([2H])c2c(c1[2H])c([2H])c([2H])c1c2sc2c([2H])c(-c3cccc(-c4nc(-c5cccc(-c6ccccc6)c5)nc(-c5ccc6c(c5)sc5cccc(-c7ccccc7)c56)n4)c3)c([2H])c([2H])c21]

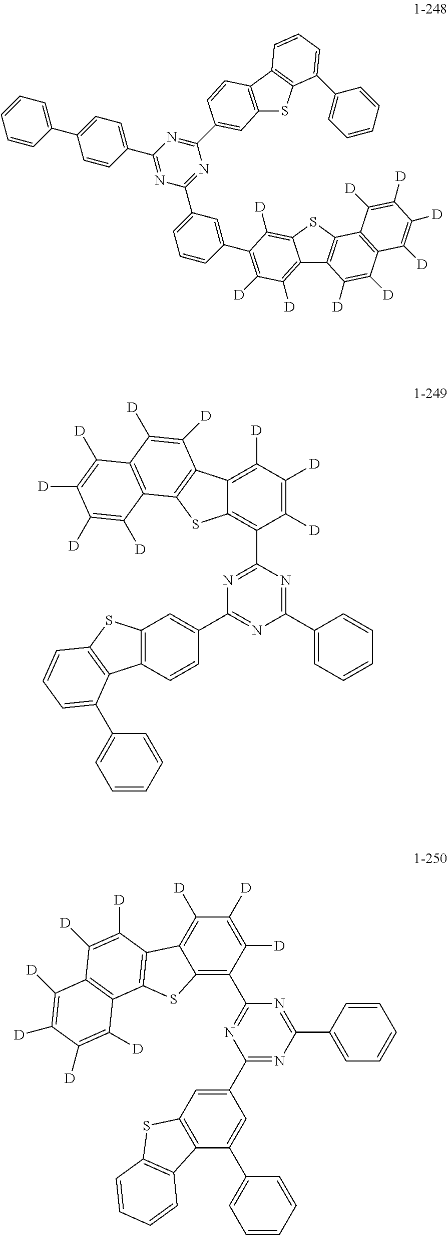 [SMILES: [2H]c1c([2H])c([2H])c2c(c1[2H])c([2H])c([2H])c1c2sc2c([2H])c(-c3cccc(-c4nc(-c5ccc(-c6ccccc6)cc5)nc(-c5ccc6c(c5)sc5c(-c7ccccc7)cccc56)n4)c3)c([2H])c([2H])c21 ; [2H]c1c([2H])c([2H])c2c(sc3c4c([2H])c([2H])c([2H])c([2H])c4c([2H])c([2H])c32)c1-c1nc(-c2ccccc2)nc(-c2ccc3c(c2)sc2cccc(-c4ccccc4)c23)n1 ; [2H]c1c([2H])c([2H])c2c(sc3c4c([2H])c([2H])c([2H])c([2H])c4c([2H])c([2H])c32)c1-c1nc(-c2ccccc2)nc(-c2cc(-c3ccccc3)c3c(c2)sc2ccccc23)n1]

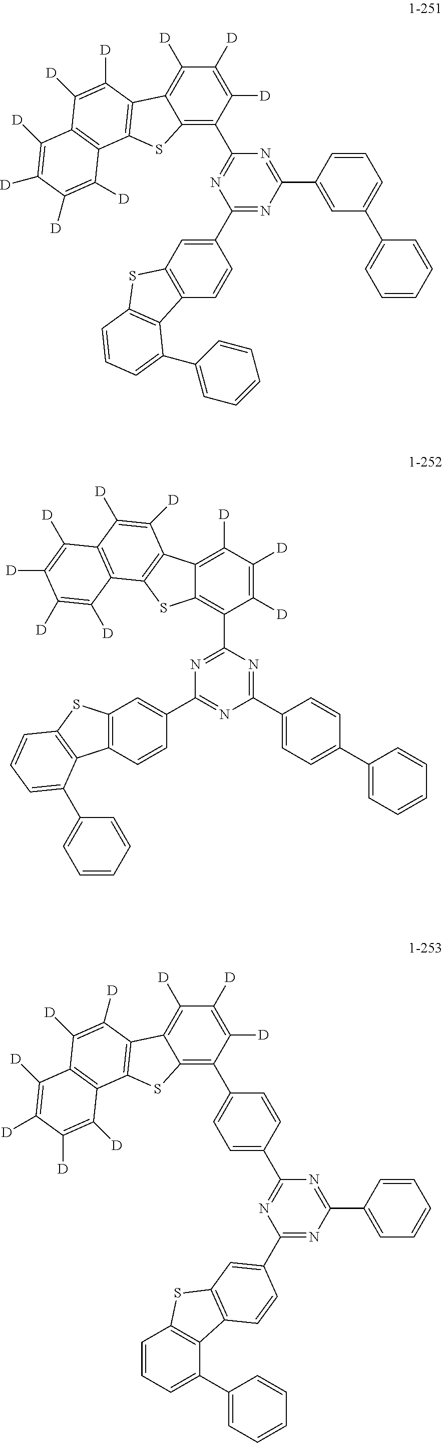 [SMILES: [2H]c1c([2H])c([2H])c2c(sc3c4c([2H])c([2H])c([2H])c([2H])c4c([2H])c([2H])c32)c1-c1nc(-c2cccc(-c3ccccc3)c2)nc(-c2ccc3c(c2)sc2cccc(-c4ccccc4)c23)n1 ; [2H]c1c([2H])c([2H])c2c(sc3c4c([2H])c([2H])c([2H])c([2H])c4c([2H])c([2H])c32)c1-c1nc(-c2ccc(-c3ccccc3)cc2)nc(-c2ccc3c(c2)sc2cccc(-c4ccccc4)c23)n1 ; [2H]c1c([2H])c([2H])c2c(sc3c4c([2H])c([2H])c([2H])c([2H])c4c([2H])c([2H])c32)c1-c1ccc(-c2nc(-c3ccccc3)nc(-c3ccc4c(c3)sc3cccc(-c5ccccc5)c34)n2)cc1]

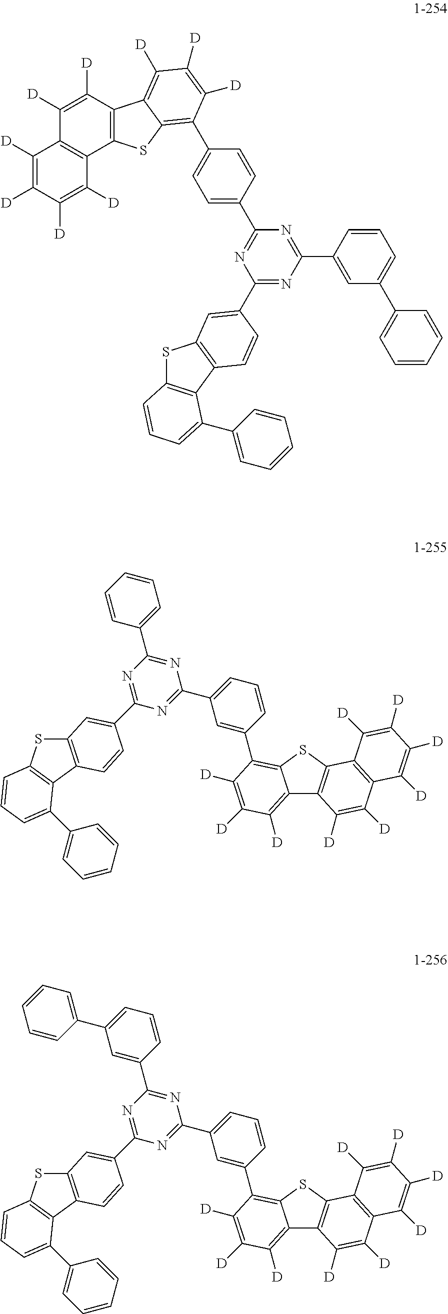 [SMILES: [2H]c1c([2H])c([2H])c2c(sc3c4c([2H])c([2H])c([2H])c([2H])c4c([2H])c([2H])c32)c1-c1ccc(-c2nc(-c3cccc(-c4ccccc4)c3)nc(-c3ccc4c(c3)sc3cccc(-c5ccccc5)c34)n2)cc1 ; [2H]c1c([2H])c([2H])c2c(sc3c4c([2H])c([2H])c([2H])c([2H])c4c([2H])c([2H])c32)c1-c1cccc(-c2nc(-c3ccccc3)nc(-c3ccc4c(c3)sc3cccc(-c5ccccc5)c34)n2)c1 ; [2H]c1c([2H])c([2H])c2c(sc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3cccc(-c5ccccc5)c34)n2)c1]

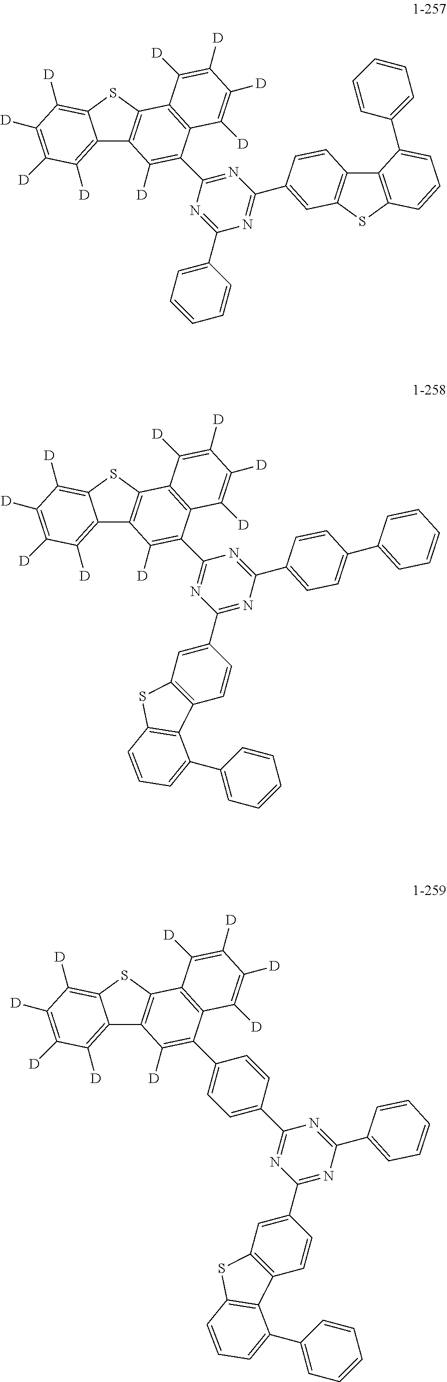 [SMILES: [2H]c1c([2H])c([2H])c2c(sc3c4c([2H])c([2H])c([2H])c([2H])c4c(-c4nc(-c5ccccc5)nc(-c5ccc6c(c5)sc5cccc(-c7ccccc7)c56)n4)c([2H])c32)c1[2H] ; [2H]c1c([2H])c([2H])c2c(sc3c4c([2H])c([2H])c([2H])c([2H])c4c(-c4nc(-c5ccc(-c6ccccc6)cc5)nc(-c5ccc6c(c5)sc5cccc(-c7ccccc7)c56)n4)c([2H])c32)c1[2H] ; [2H]c1c([2H])c([2H])c2c(sc3c4c([2H])c([2H])c([2H])c([2H])c4c(-c4ccc(-c5nc(-c6ccccc6)nc(-c6ccc7c(c6)sc6cccc(-c8ccccc8)c67)n5)cc4)c([2H])c32)c1[2H]]

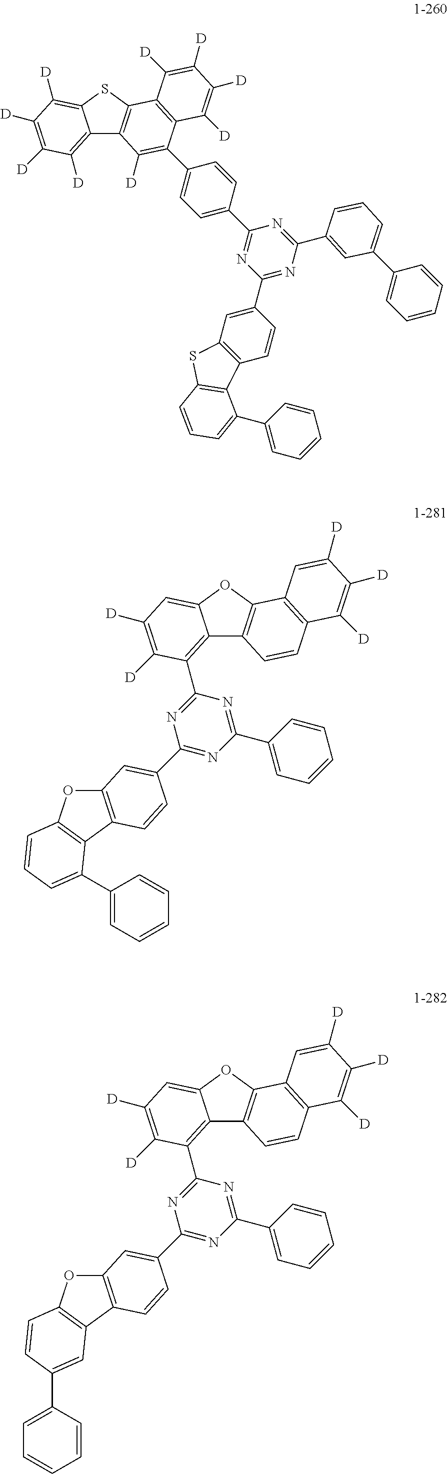 [SMILES: [2H]c1c([2H])c([2H])c2c(sc3c4c([2H])c([2H])c([2H])c([2H])c4c(-c4ccc(-c5nc(-c6cccc(-c7ccccc7)c6)nc(-c6ccc7c(c6)sc6cccc(-c8ccccc8)c67)n5)cc4)c([2H])c32)c1[2H] ; [2H]c1cc2c(ccc3c2oc2cc([2H])c([2H])c(-c4nc(-c5ccccc5)nc(-c5ccc6c(c5)oc5cccc(-c7ccccc7)c56)n4)c23)c([2H])c1[2H] ; [2H]c1cc2c(ccc3c2oc2cc([2H])c([2H])c(-c4nc(-c5ccccc5)nc(-c5ccc6c(c5)oc5ccc(-c7ccccc7)cc56)n4)c23)c([2H])c1[2H]]

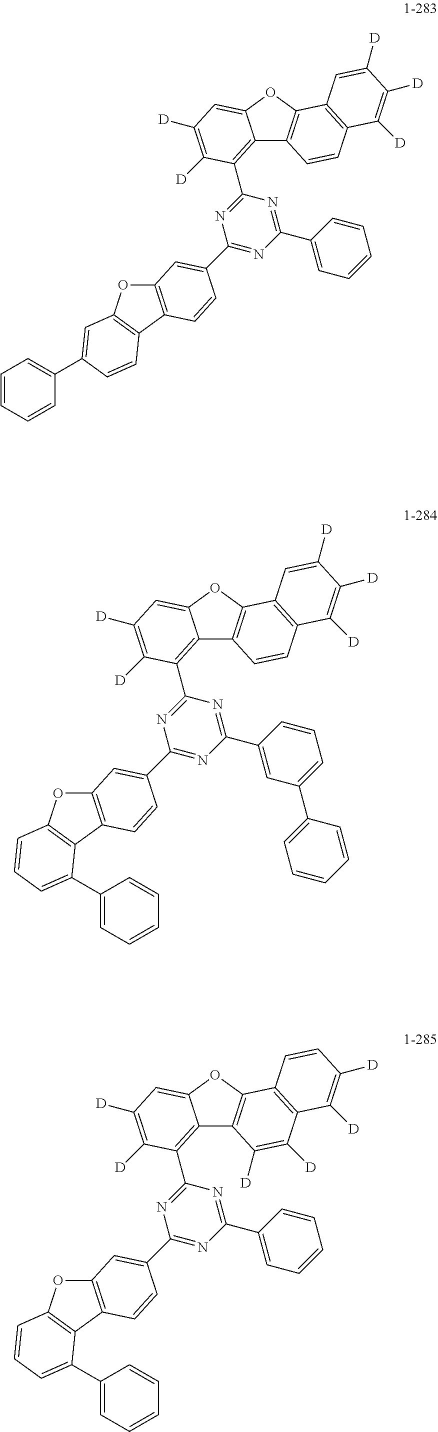 [SMILES: [2H]c1cc2c(ccc3c2oc2cc([2H])c([2H])c(-c4nc(-c5ccccc5)nc(-c5ccc6c(c5)oc5cc(-c7ccccc7)ccc56)n4)c23)c([2H])c1[2H] ; [2H]c1cc2c(ccc3c2oc2cc([2H])c([2H])c(-c4nc(-c5cccc(-c6ccccc6)c5)nc(-c5ccc6c(c5)oc5cccc(-c7ccccc7)c56)n4)c23)c([2H])c1[2H] ; [2H]c1cc2oc3c4ccc([2H])c([2H])c4c([2H])c([2H])c3c2c(-c2nc(-c3ccccc3)nc(-c3ccc4c(c3)oc3cccc(-c5ccccc5)c34)n2)c1[2H]]

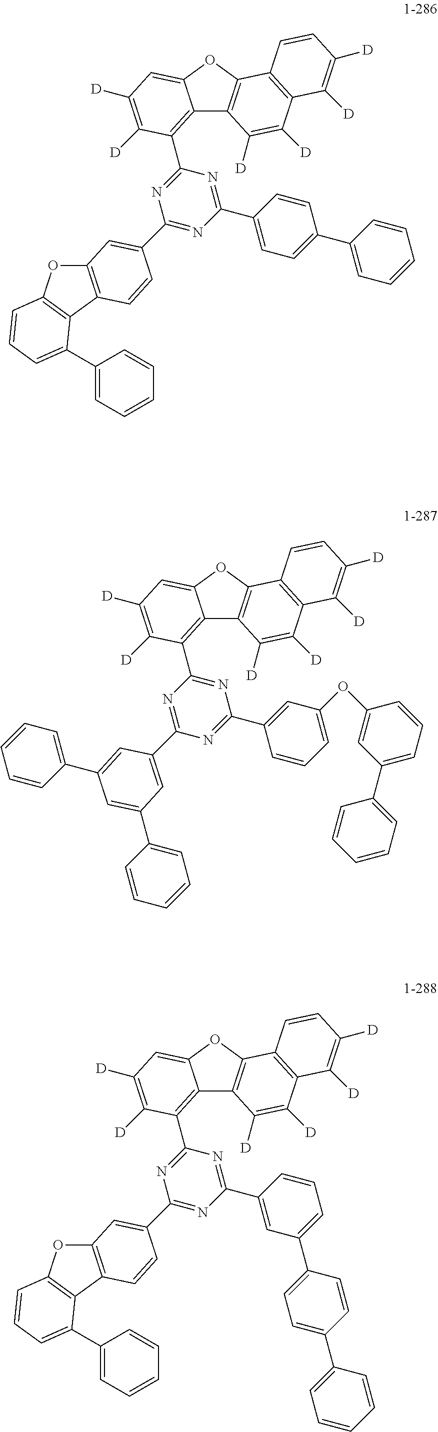 [SMILES: [2H]c1cc2oc3c4ccc([2H])c([2H])c4c([2H])c([2H])c3c2c(-c2nc(-c3ccc(-c4ccccc4)cc3)nc(-c3ccc4c(c3)oc3cccc(-c5ccccc5)c34)n2)c1[2H] ; [2H]c1cc2oc3c4ccc([2H])c([2H])c4c([2H])c([2H])c3c2c(-c2nc(-c3cccc(Oc4cccc(-c5ccccc5)c4)c3)nc(-c3cc(-c4ccccc4)cc(-c4ccccc4)c3)n2)c1[2H] ; [2H]c1cc2oc3c4ccc([2H])c([2H])c4c([2H])c([2H])c3c2c(-c2nc(-c3cccc(-c4ccc(-c5ccccc5)cc4)c3)nc(-c3ccc4c(c3)oc3cccc(-c5ccccc5)c34)n2)c1[2H]]

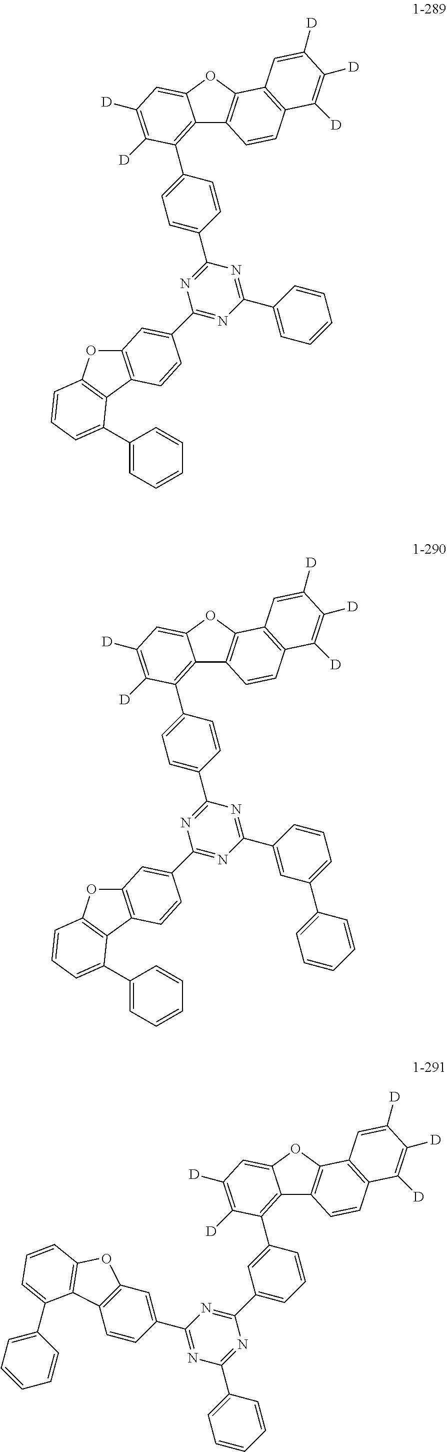 [SMILES: [2H]c1cc2c(ccc3c2oc2cc([2H])c([2H])c(-c4ccc(-c5nc(-c6ccccc6)nc(-c6ccc7c(c6)oc6cccc(-c8ccccc8)c67)n5)cc4)c23)c([2H])c1[2H] ; [2H]c1cc2c(ccc3c2oc2cc([2H])c([2H])c(-c4ccc(-c5nc(-c6cccc(-c7ccccc7)c6)nc(-c6ccc7c(c6)oc6cccc(-c8ccccc8)c67)n5)cc4)c23)c([2H])c1[2H] ; [2H]c1cc2c(ccc3c2oc2cc([2H])c([2H])c(-c4cccc(-c5nc(-c6ccccc6)nc(-c6ccc7c(c6)oc6cccc(-c8ccccc8)c67)n5)c4)c23)c([2H])c1[2H]]

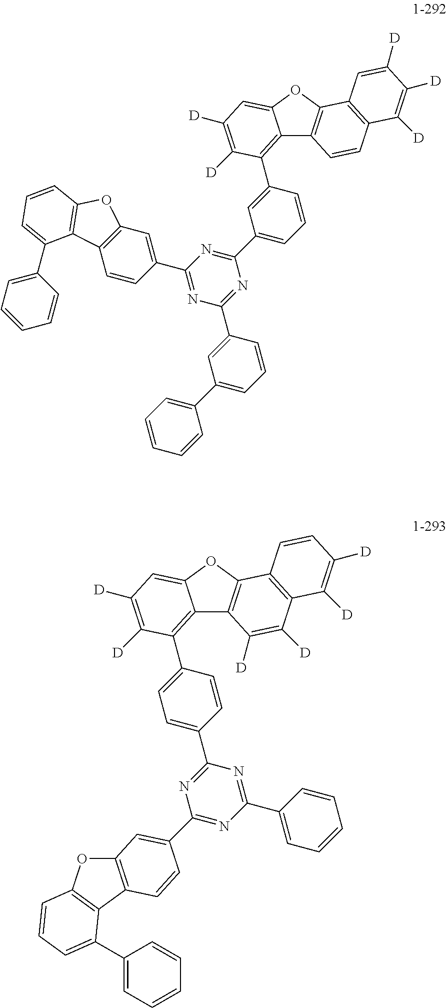 [SMILES: [2H]c1cc2c(ccc3c2oc2cc([2H])c([2H])c(-c4cccc(-c5nc(-c6cccc(-c7ccccc7)c6)nc(-c6ccc7c(c6)oc6cccc(-c8ccccc8)c67)n5)c4)c23)c([2H])c1[2H] ; [2H]c1cc2oc3c4ccc([2H])c([2H])c4c([2H])c([2H])c3c2c(-c2ccc(-c3nc(-c4ccccc4)nc(-c4ccc5c(c4)oc4cccc(-c6ccccc6)c45)n3)cc2)c1[2H]]

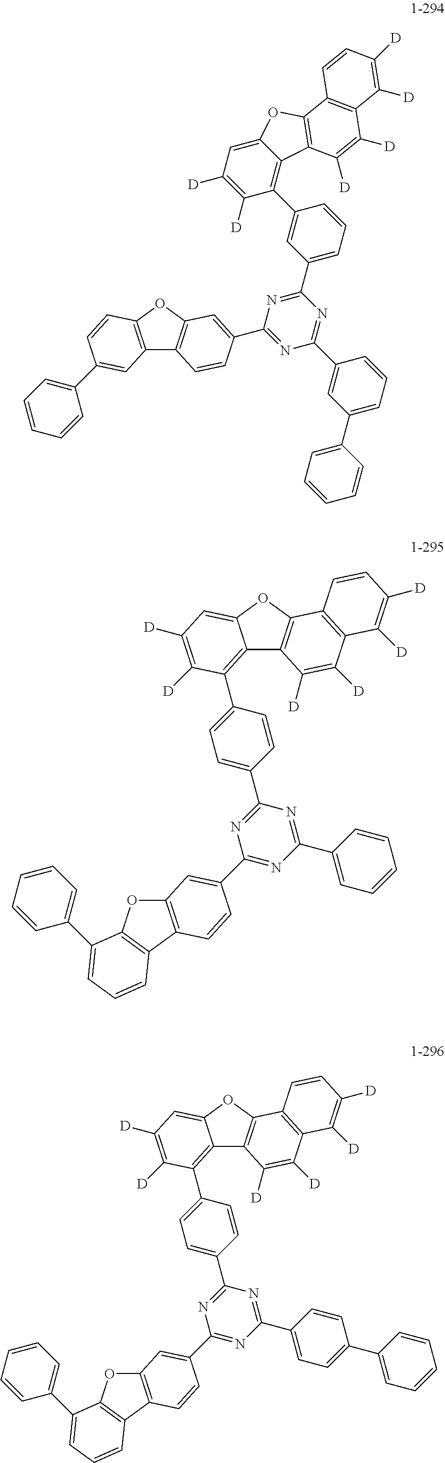 [SMILES: [2H]c1cc2oc3c4ccc([2H])c([2H])c4c([2H])c([2H])c3c2c(-c2cccc(-c3nc(-c4cccc(-c5ccccc5)c4)nc(-c4ccc5c(c4)oc4ccc(-c6ccccc6)cc45)n3)c2)c1[2H] ; [2H]c1cc2oc3c4ccc([2H])c([2H])c4c([2H])c([2H])c3c2c(-c2ccc(-c3nc(-c4ccccc4)nc(-c4ccc5c(c4)oc4c(-c6ccccc6)cccc45)n3)cc2)c1[2H] ; [2H]c1cc2oc3c4ccc([2H])c([2H])c4c([2H])c([2H])c3c2c(-c2ccc(-c3nc(-c4ccc(-c5ccccc5)cc4)nc(-c4ccc5c(c4)oc4c(-c6ccccc6)cccc45)n3)cc2)c1[2H]]

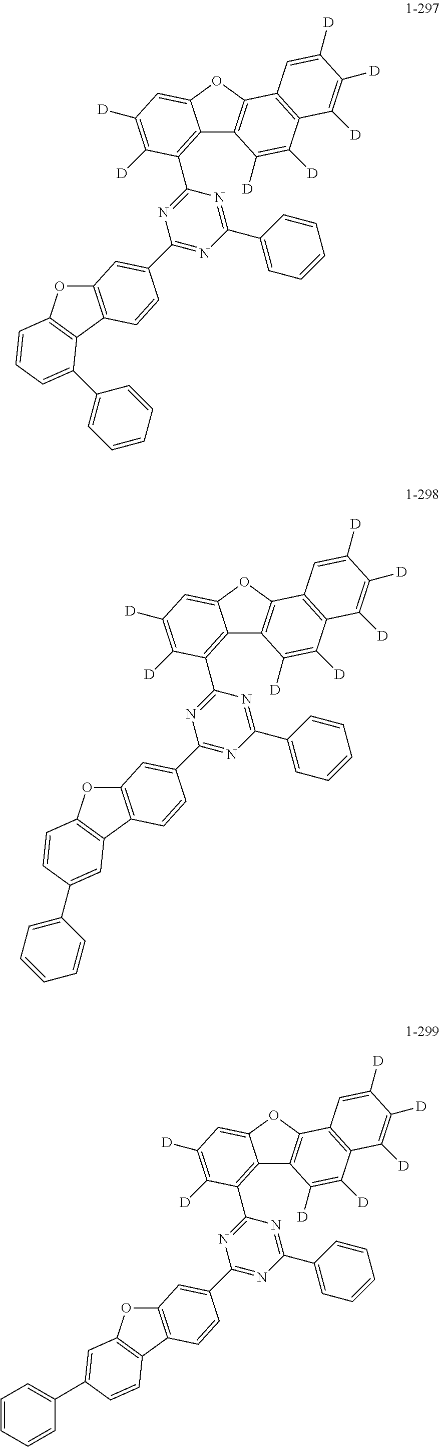 [SMILES: [2H]c1cc2c(c([2H])c1[2H])c([2H])c([2H])c1c2oc2cc([2H])c([2H])c(-c3nc(-c4ccccc4)nc(-c4ccc5c(c4)oc4cccc(-c6ccccc6)c45)n3)c21 ; [2H]c1cc2c(c([2H])c1[2H])c([2H])c([2H])c1c2oc2cc([2H])c([2H])c(-c3nc(-c4ccccc4)nc(-c4ccc5c(c4)oc4ccc(-c6ccccc6)cc45)n3)c21 ; [2H]c1cc2c(c([2H])c1[2H])c([2H])c([2H])c1c2oc2cc([2H])c([2H])c(-c3nc(-c4ccccc4)nc(-c4ccc5c(c4)oc4cc(-c6ccccc6)ccc45)n3)c21]

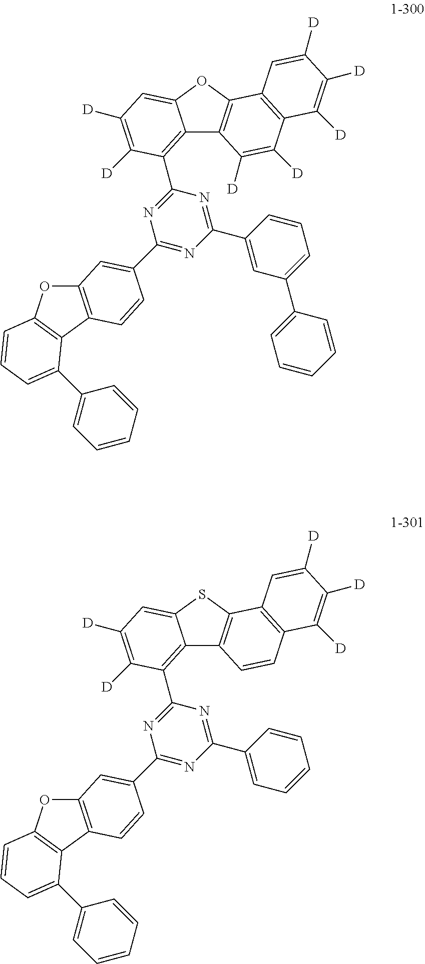 [SMILES: [2H]c1cc2c(c([2H])c1[2H])c([2H])c([2H])c1c2oc2cc([2H])c([2H])c(-c3nc(-c4cccc(-c5ccccc5)c4)nc(-c4ccc5c(c4)oc4cccc(-c6ccccc6)c45)n3)c21 ; [2H]c1cc2c(ccc3c2sc2cc([2H])c([2H])c(-c4nc(-c5ccccc5)nc(-c5ccc6c(c5)oc5cccc(-c7ccccc7)c56)n4)c23)c([2H])c1[2H]]

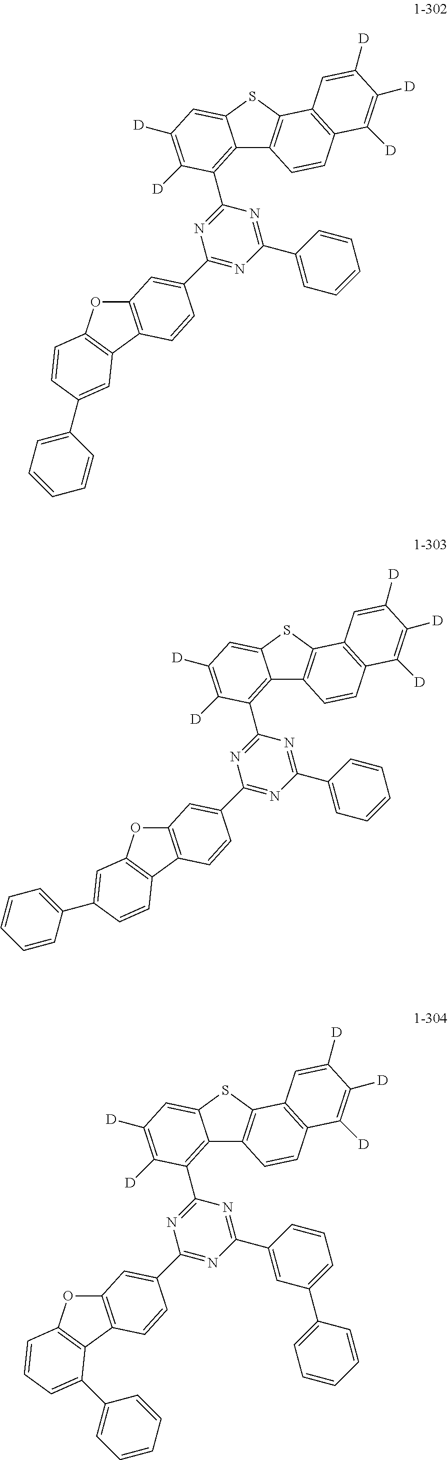 [SMILES: [2H]c1cc2c(ccc3c2sc2cc([2H])c([2H])c(-c4nc(-c5ccccc5)nc(-c5ccc6c(c5)oc5ccc(-c7ccccc7)cc56)n4)c23)c([2H])c1[2H] ; [2H]c1cc2c(ccc3c2sc2cc([2H])c([2H])c(-c4nc(-c5ccccc5)nc(-c5ccc6c(c5)oc5cc(-c7ccccc7)ccc56)n4)c23)c([2H])c1[2H] ; [2H]c1cc2c(ccc3c2sc2cc([2H])c([2H])c(-c4nc(-c5cccc(-c6ccccc6)c5)nc(-c5ccc6c(c5)oc5cccc(-c7ccccc7)c56)n4)c23)c([2H])c1[2H]]

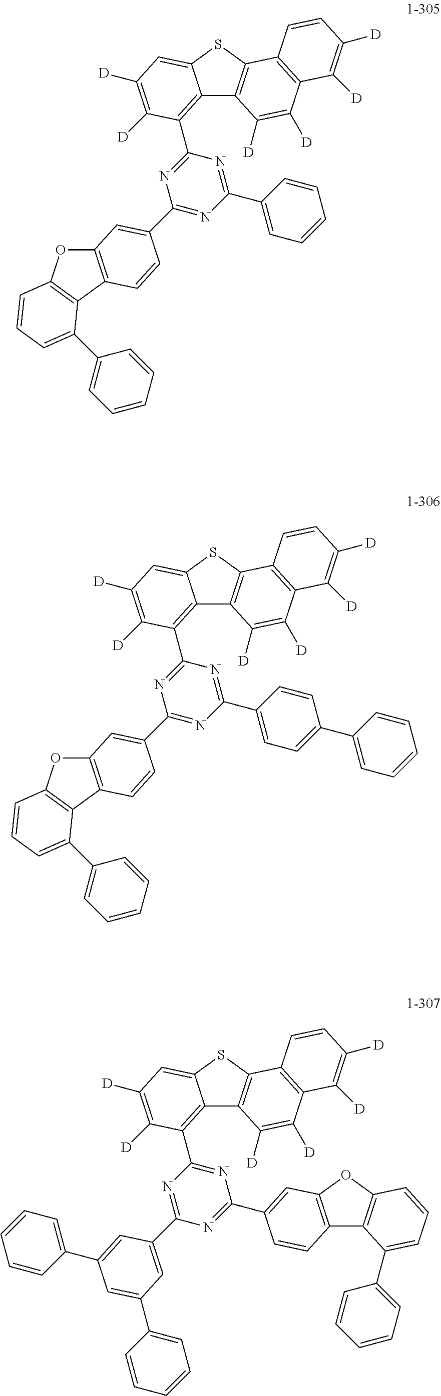 [SMILES: [2H]c1cc2sc3c4ccc([2H])c([2H])c4c([2H])c([2H])c3c2c(-c2nc(-c3ccccc3)nc(-c3ccc4c(c3)oc3cccc(-c5ccccc5)c34)n2)c1[2H] ; [2H]c1cc2sc3c4ccc([2H])c([2H])c4c([2H])c([2H])c3c2c(-c2nc(-c3ccc(-c4ccccc4)cc3)nc(-c3ccc4c(c3)oc3cccc(-c5ccccc5)c34)n2)c1[2H] ; [2H]c1cc2sc3c4ccc([2H])c([2H])c4c([2H])c([2H])c3c2c(-c2nc(-c3cc(-c4ccccc4)cc(-c4ccccc4)c3)nc(-c3ccc4c(c3)oc3cccc(-c5ccccc5)c34)n2)c1[2H]]

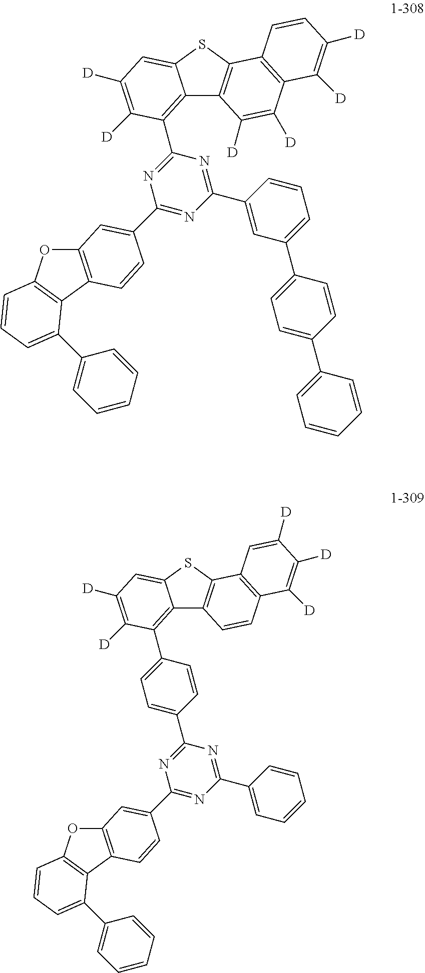 [SMILES: [2H]c1cc2sc3c4ccc([2H])c([2H])c4c([2H])c([2H])c3c2c(-c2nc(-c3cccc(-c4ccc(-c5ccccc5)cc4)c3)nc(-c3ccc4c(c3)oc3cccc(-c5ccccc5)c34)n2)c1[2H] ; [2H]c1cc2c(ccc3c2sc2cc([2H])c([2H])c(-c4ccc(-c5nc(-c6ccccc6)nc(-c6ccc7c(c6)oc6cccc(-c8ccccc8)c67)n5)cc4)c23)c([2H])c1[2H]]

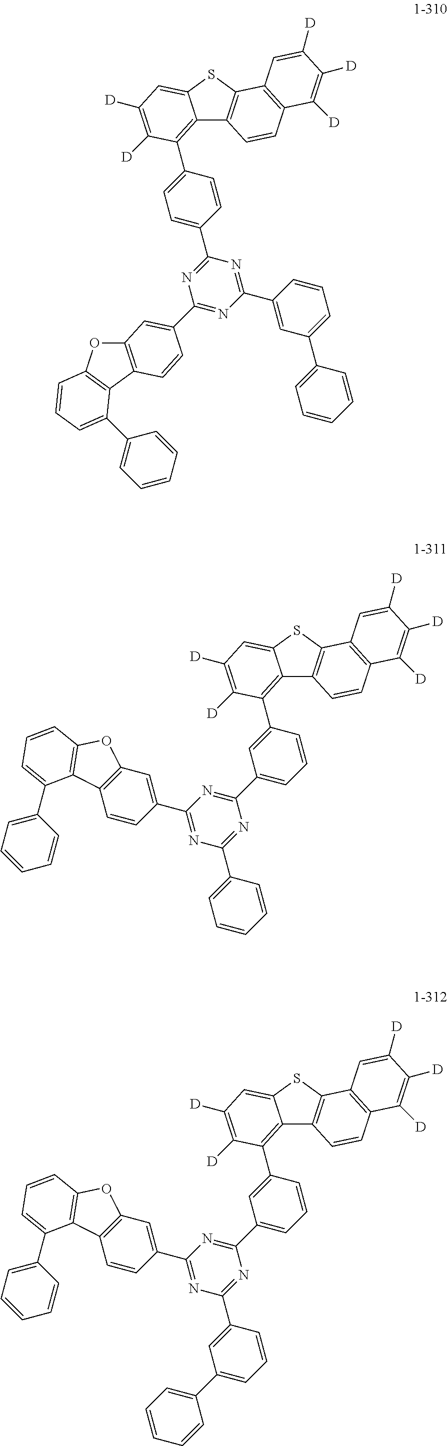 [SMILES: [2H]c1cc2c(ccc3c2sc2cc([2H])c([2H])c(-c4ccc(-c5nc(-c6cccc(-c7ccccc7)c6)nc(-c6ccc7c(c6)oc6cccc(-c8ccccc8)c67)n5)cc4)c23)c([2H])c1[2H] ; [2H]c1cc2c(ccc3c2sc2cc([2H])c([2H])c(-c4cccc(-c5nc(-c6ccccc6)nc(-c6ccc7c(c6)oc6cccc(-c8ccccc8)c67)n5)c4)c23)c([2H])c1[2H] ; [2H]c1cc2c(ccc3c2sc2cc([2H])c([2H])c(-c4cccc(-c5nc(-c6cccc(-c7ccccc7)c6)nc(-c6ccc7c(c6)oc6cccc(-c8ccccc8)c67)n5)c4)c23)c([2H])c1[2H]]

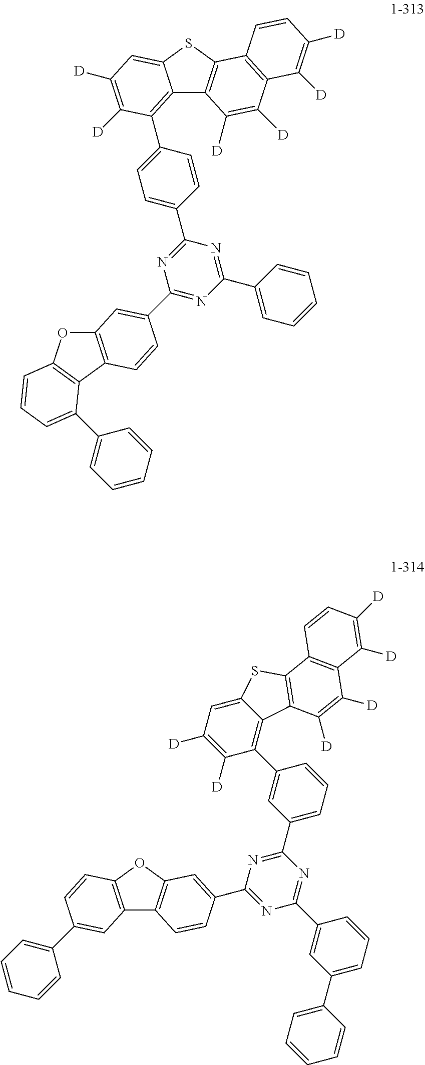 [SMILES: [2H]c1cc2sc3c4ccc([2H])c([2H])c4c([2H])c([2H])c3c2c(-c2ccc(-c3nc(-c4ccccc4)nc(-c4ccc5c(c4)oc4cccc(-c6ccccc6)c45)n3)cc2)c1[2H] ; [2H]c1cc2sc3c4ccc([2H])c([2H])c4c([2H])c([2H])c3c2c(-c2cccc(-c3nc(-c4cccc(-c5ccccc5)c4)nc(-c4ccc5c(c4)oc4ccc(-c6ccccc6)cc45)n3)c2)c1[2H]]

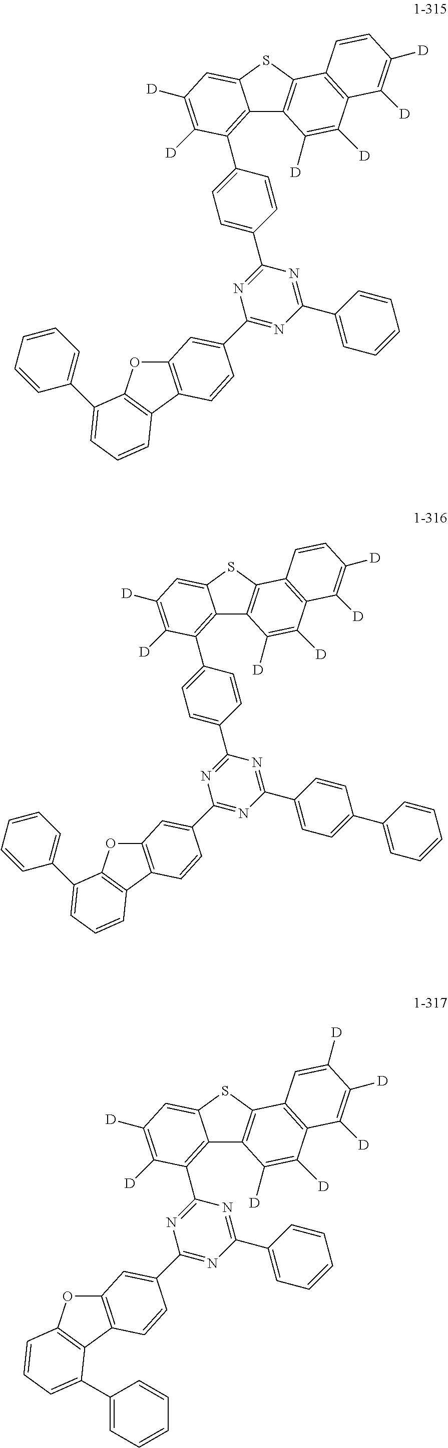 [SMILES: [2H]c1cc2sc3c4ccc([2H])c([2H])c4c([2H])c([2H])c3c2c(-c2ccc(-c3nc(-c4ccccc4)nc(-c4ccc5c(c4)oc4c(-c6ccccc6)cccc45)n3)cc2)c1[2H] ; [2H]c1cc2sc3c4ccc([2H])c([2H])c4c([2H])c([2H])c3c2c(-c2ccc(-c3nc(-c4ccc(-c5ccccc5)cc4)nc(-c4ccc5c(c4)oc4c(-c6ccccc6)cccc45)n3)cc2)c1[2H] ; [2H]c1cc2c(c([2H])c1[2H])c([2H])c([2H])c1c2sc2cc([2H])c([2H])c(-c3nc(-c4ccccc4)nc(-c4ccc5c(c4)oc4cccc(-c6ccccc6)c45)n3)c21]

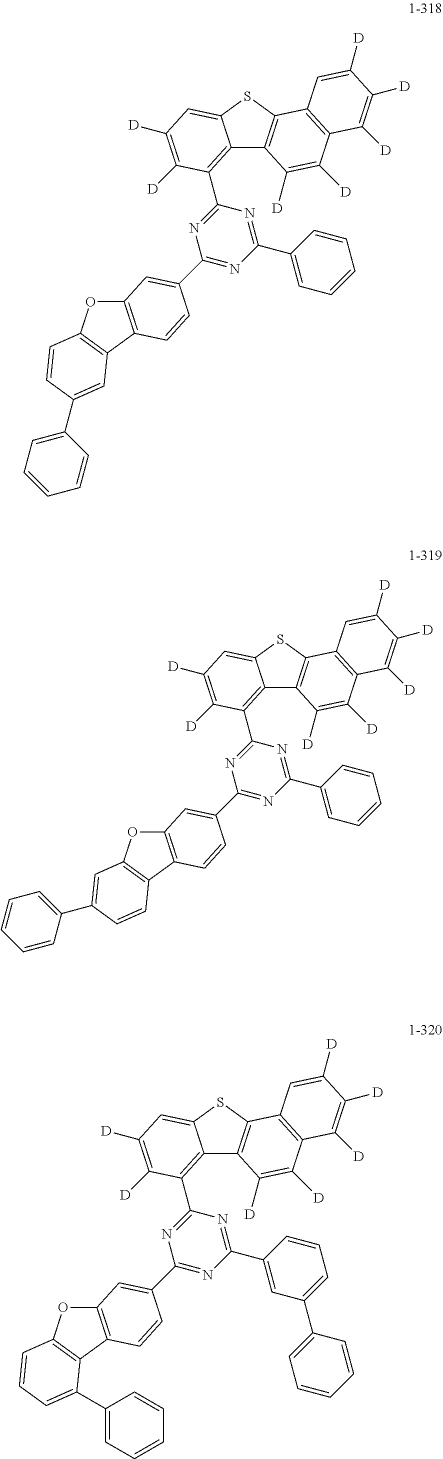 [SMILES: [2H]c1cc2c(c([2H])c1[2H])c([2H])c([2H])c1c2sc2cc([2H])c([2H])c(-c3nc(-c4ccccc4)nc(-c4ccc5c(c4)oc4ccc(-c6ccccc6)cc45)n3)c21 ; [2H]c1cc2c(c([2H])c1[2H])c([2H])c([2H])c1c2sc2cc([2H])c([2H])c(-c3nc(-c4ccccc4)nc(-c4ccc5c(c4)oc4cc(-c6ccccc6)ccc45)n3)c21 ; [2H]c1cc2c(c([2H])c1[2H])c([2H])c([2H])c1c2sc2cc([2H])c([2H])c(-c3nc(-c4cccc(-c5ccccc5)c4)nc(-c4ccc5c(c4)oc4cccc(-c6ccccc6)c45)n3)c21]

In another embodiment of the present specification, a first electrode; a second electrode; and one or more organic material layers disposed between the first electrode and the second electrode are included in an organic light emitting device, and one or more of the organic material layers include the heterocyclic compound of Formula 1.

In one embodiment of the present specification, the organic material layer including the heterocyclic compound may further include a compound of Formula 2 below.

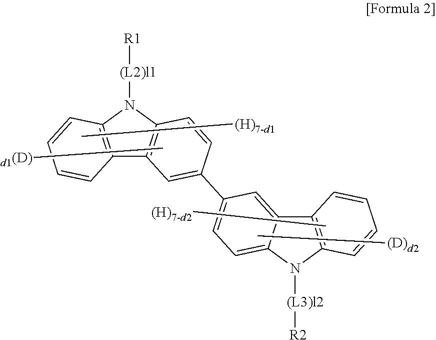

In Formula 2,
L2 and L3 are each independently a direct bond; or a substituted or unsubstituted arylene group having 6 to 60 carbon atoms,
each of 11 and 12 is an integer of 1 to 3, and when it is 2 or higher, each substituent in the parentheses is the same or different,
R1 and R2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group 6 to 60 carbon atoms; or a substituted or unsubstituted heteroaryl group having 2 to 60 carbon atoms,
H is hydrogen, and D is deuterium, and
d1 and d2 are each independently an integer of 0 to 7.

An organic light emitting device including both the heterocyclic compound of Formula 1 and the compound of Formula 2 exhibits more excellent efficiency and lifetime effects. This result can be expected because, when both compounds are included, an exciplex phenomenon occurs.

The exciplex phenomenon is a phenomenon in which energy with a HOMO level of a donor (p-host) and a LUMO level of an acceptor (n-host) is emitted due to electron exchange between two molecules. When the exciplex phenomenon between two molecules occurs, reverse intersystem crossing (RISC) occurs, and due to RISC, the internal quantum efficiency of fluorescence may increase up to 100%. When a donor (p-host) with excellent hole transport ability and an acceptor (n-host) with excellent electron transport ability are used as hosts for an emissive layer, since holes are injected into the p-host and electrons are injected into the n-host, a driving voltage may be lowered, thereby helping to improve the lifetime. In the present application, as the compound of Formula 2 serves as a donor and the compound of Formula 1 serves as an acceptor, it can be confirmed that when used as the hosts for the emissive layer, an excellent device characteristic is exhibited.

In one embodiment of the present specification, L2 and L3 are each independently a direct bond; or a substituted or unsubstituted C6 to C30 arylene group.

In one embodiment of the present specification, L2 and L3 are each independently a direct bond; or a C6 to C30 arylene group substituted or unsubstituted with deuterium.

In one embodiment of the present specification, L2 and L3 are each independently a direct bond; or a C6 to C12 arylene group substituted or unsubstituted with deuterium.

In one embodiment of the present specification, L2 and L3 are each independently a direct bond; a phenylene group substituted or unsubstituted with deuterium; or a biphenylene group substituted or unsubstituted with deuterium.

In one embodiment of the present specification, R1 and R2 are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In one embodiment of the present specification, R1 and R2 are each independently a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In one embodiment of the present specification, R1 and R2 are each independently a substituted or unsubstituted C1 to C30 alkyl group; a substituted or unsubstituted C3 to C30 cycloalkyl group; a substituted or unsubstituted C6 to C30 aryl group; or a substituted or unsubstituted C2 to C30 heteroaryl group.

In one embodiment of the present specification, R1 and R2 are each independently a substituted or unsubstituted C6 to C30 aryl group; or a substituted or unsubstituted C2 to C30 heteroaryl group.

In one embodiment of the present specification, R1 and R2 are each independently a substituted or unsubstituted C6 to C30 aryl group; or a substituted or unsubstituted C2 to C30 heteroaryl group including O or S.

In one embodiment of the present specification, R1 and R2 are each independently a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted fluorene group; a substituted or unsubstituted dibenzofuran group; or a substituted or unsubstituted dibenzothiophene group.

In one embodiment of the present specification, R1 and R2 are each independently a phenyl group substituted or unsubstituted with deuterium; a biphenyl group substituted or unsubstituted with deuterium; a terphenyl group substituted or unsubstituted with deuterium; a naphthyl group substituted or unsubstituted with deuterium; a triphenylene group substituted or unsubstituted with deuterium; a fluorene group substituted or unsubstituted with one or more substituents selected from deuterium and an alkyl group; a dibenzofuran group substituted or unsubstituted with deuterium; or a dibenzothiophene group substituted or unsubstituted with deuterium.

In one embodiment of the present specification, R1 and R2 are each independently a C6 to C30 aryl group substituted or unsubstituted with one or more substituents selected from deuterium and an alkyl group; or a C2 to C30 heteroaryl group substituted or unsubstituted with deuterium.

In one embodiment of the present specification, d1 and d2 may each be independently 0 or 7.

In one embodiment of the present specification, Formula 2 may be represented by Formula 2-1 or 2-2.

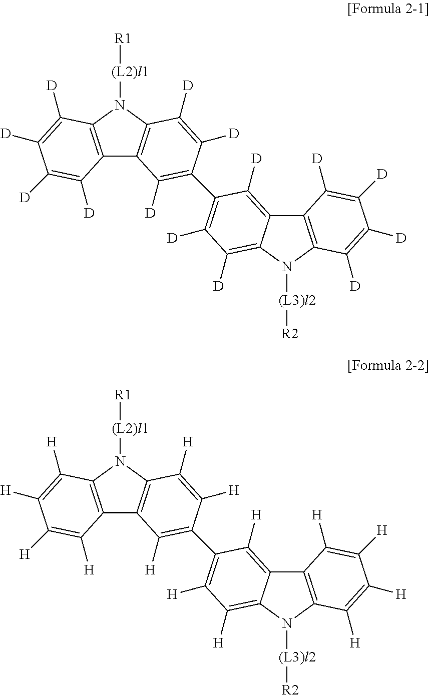

In Formulas 2-1 and 2-2, the definition of each substituent is the same as that in Formula 2.

In one embodiment of the present specification, the deuterium content in Formula 2 is 0% to 100%.

In one embodiment of the present specification, the deuterium content in Formula 2 is 0%, or 10% to 100%.

In one embodiment of the present specification, the deuterium content in Formula 2 is 0%, or 30% to 100%.

In one embodiment of the present specification, the deuterium content in Formula 2 is 0%.

In one embodiment of the present specification, the deuterium content in Formula 2 is more than 0% and 100% or less.

In one embodiment of the present specification, the deuterium content in Formula 2 is 10% to 100%.

In the organic light emitting device according to one embodiment of the present specification, the organic material layer may include a heterocyclic compound of Formula 1 in which a deuterium content is more than 0% and a compound of Formula 2 in which a deuterium content is 0%.

In the organic light emitting device according to another embodiment, the organic material layer may include a heterocyclic compound of Formula 1 in which a deuterium content is more than 0% and a compound of Formula 2 in which a deuterium content is more than 0%.

In one embodiment of the present specification, when Formula 2 includes deuterium, compared to when Formula 2 does not include deuterium, a driving voltage is lowered, and light emitting efficiency and lifetime are increased.

Likewise, in the compound of Formula 2, the electron transport moiety and the hole transport moiety, which directly exchange electrons, continuously change the vibrational frequency of the interatomic bonds in a molecule as electrons move, this affects the bonding stability between atoms in the molecule and the stability of the molecular structure. By substitution with deuterium, which has a higher molecular weight than hydrogen, the change in vibrational frequency is reduced and thus the molecular energy is lowered, thereby increasing the stability of the molecule.

Moreover, since the bond dissociation energy of carbon and deuterium is higher than that of carbon and hydrogen, the thermal stability of the molecule is increased, and thus the lifetime of the device is improved.

In one embodiment of the present specification, Formula 2 may be represented by any one of the following compounds.

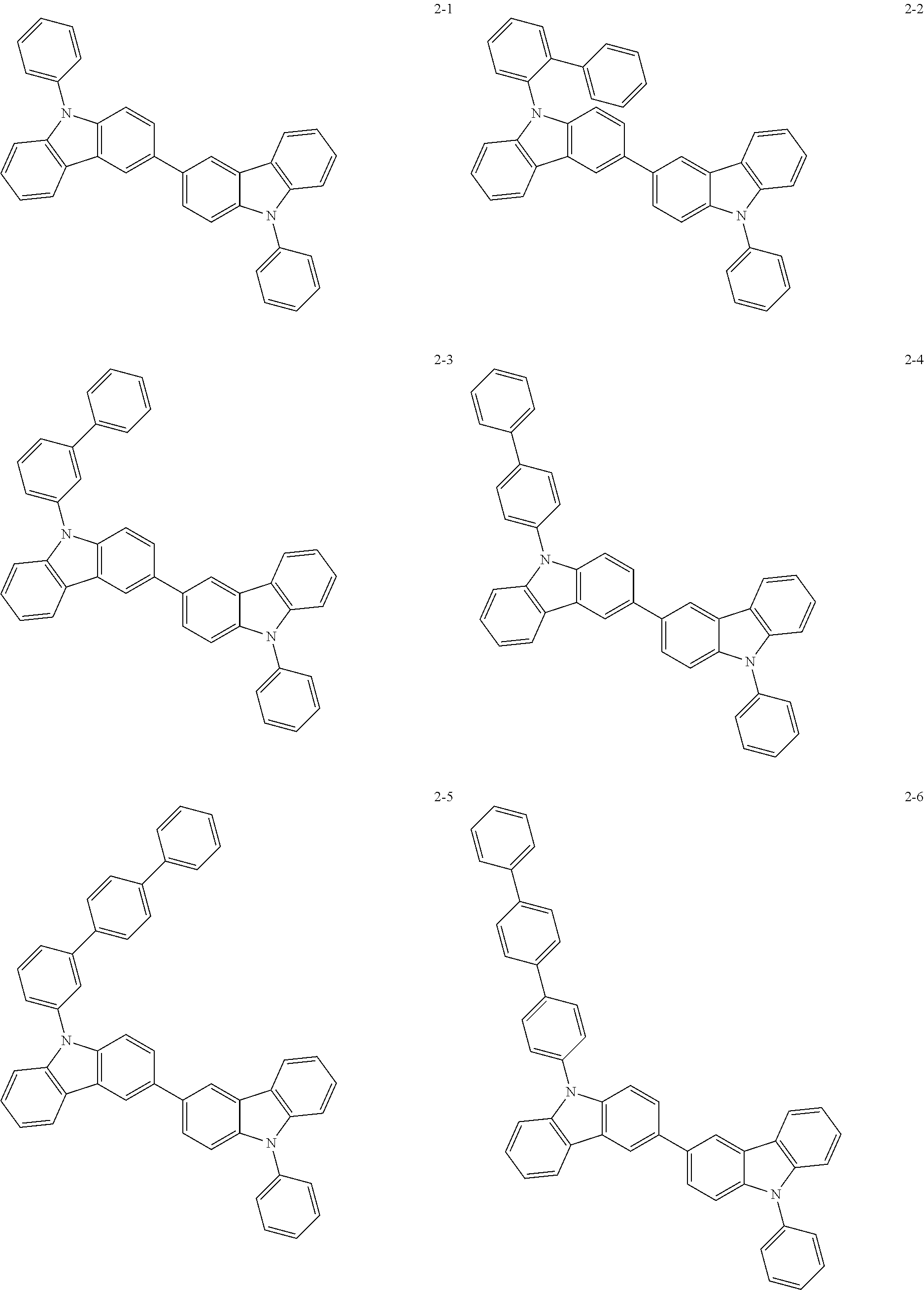 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

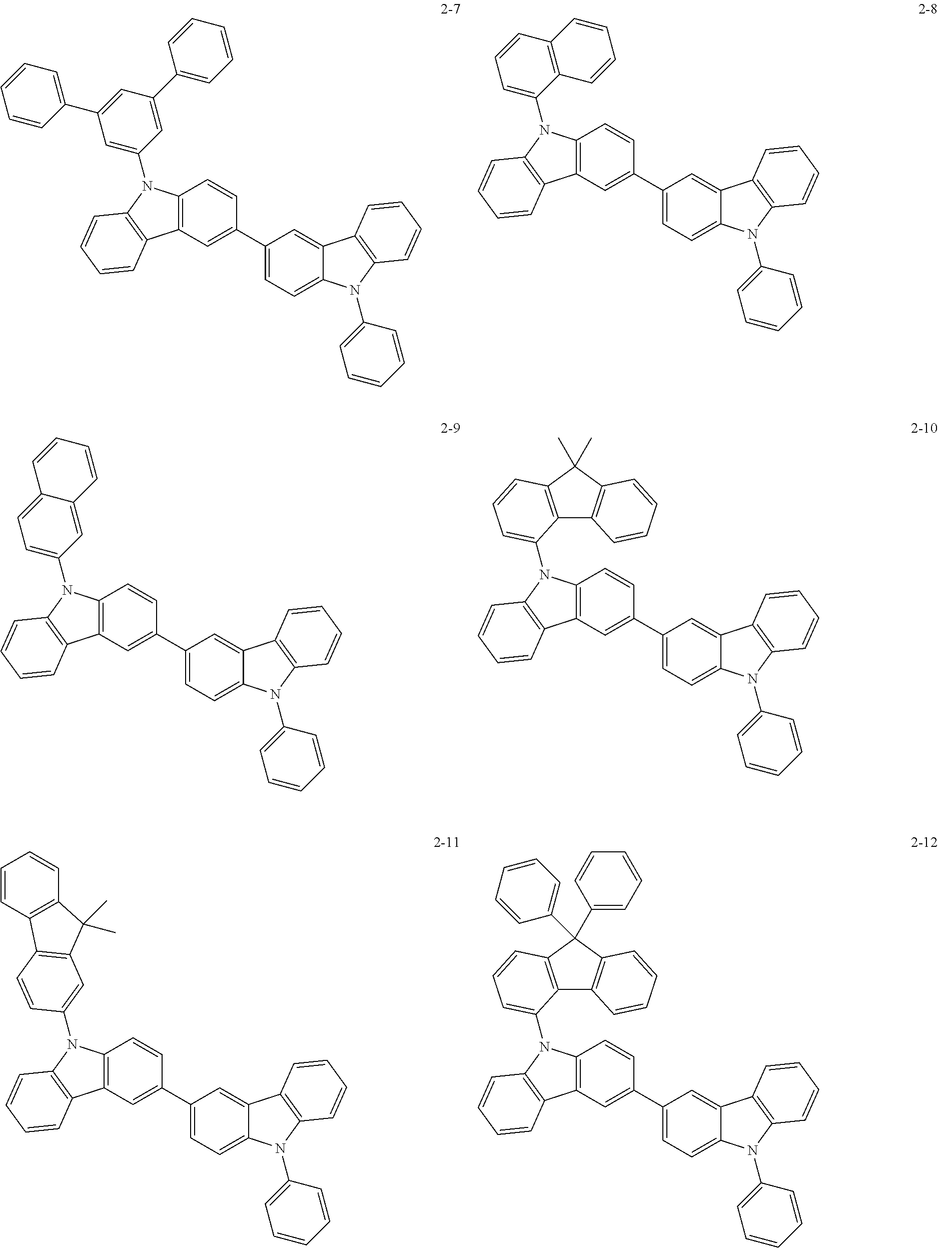 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

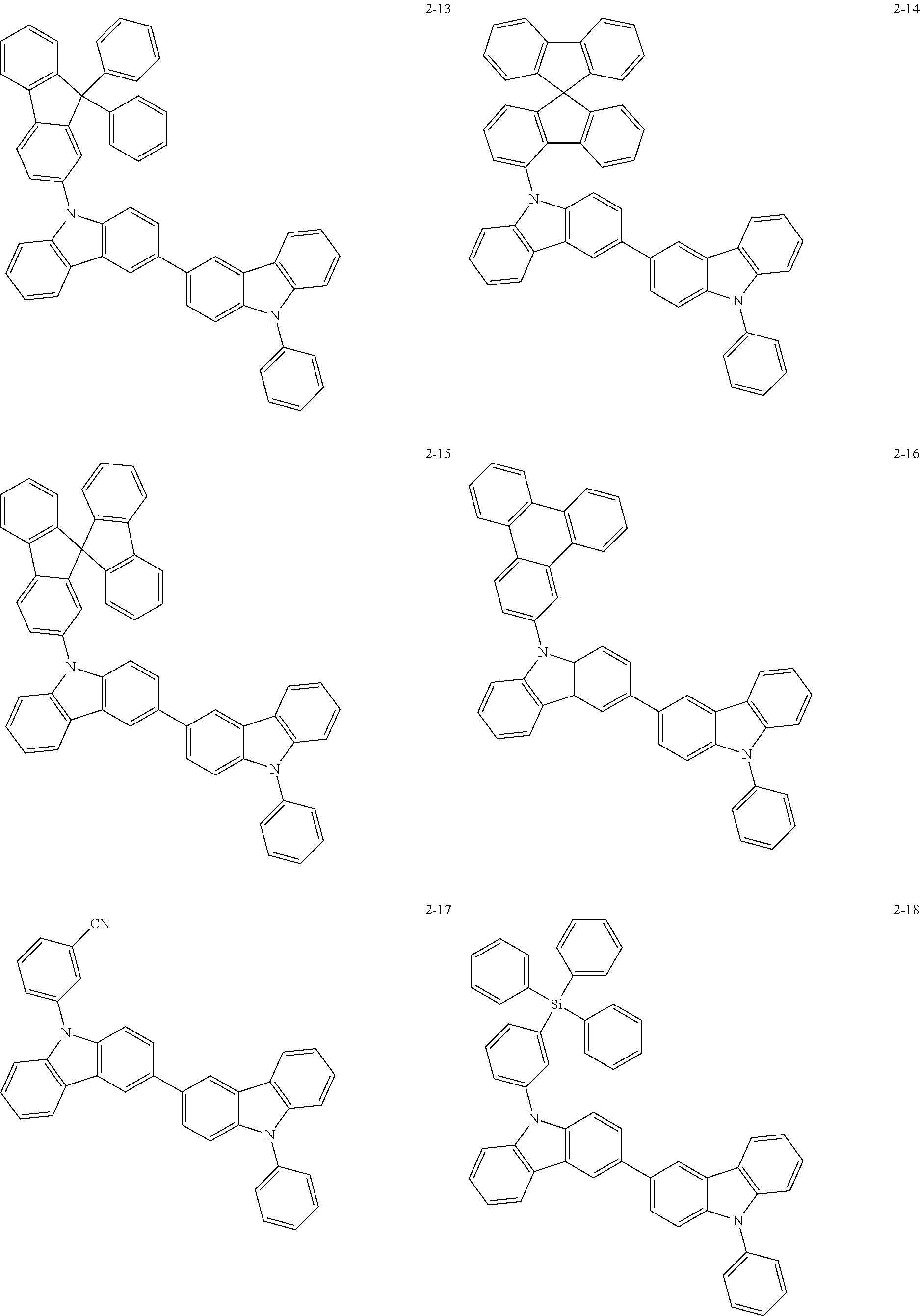

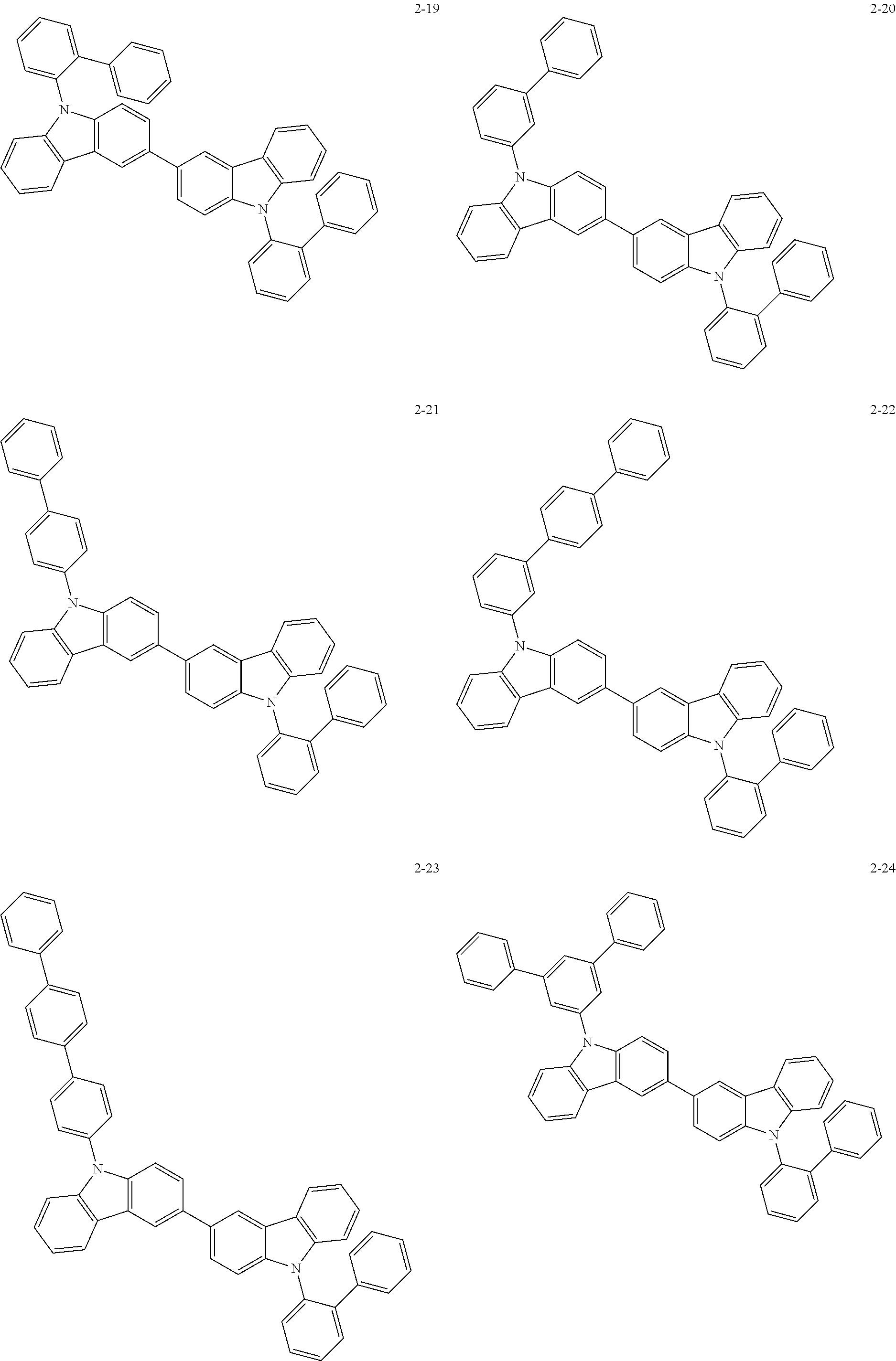

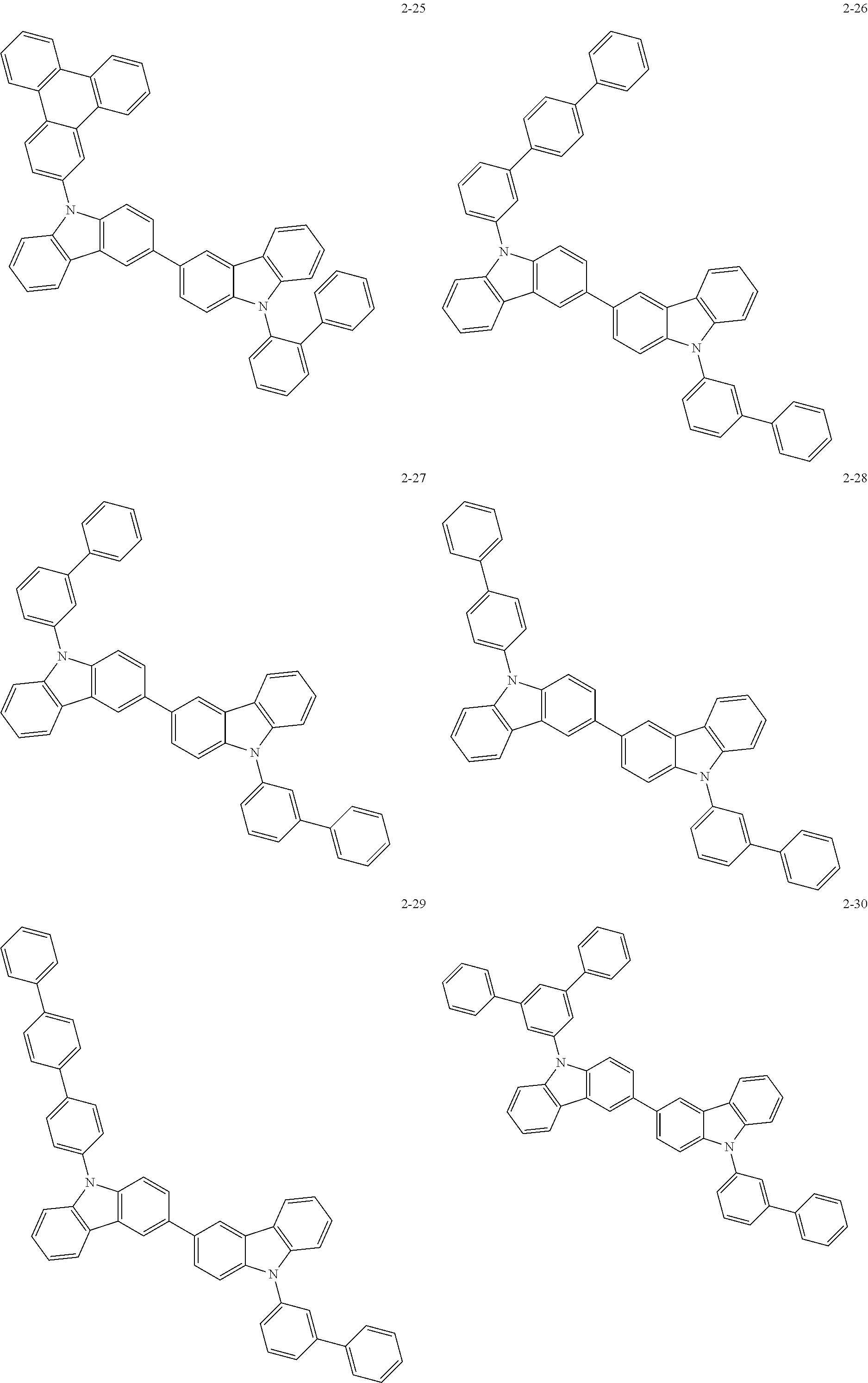

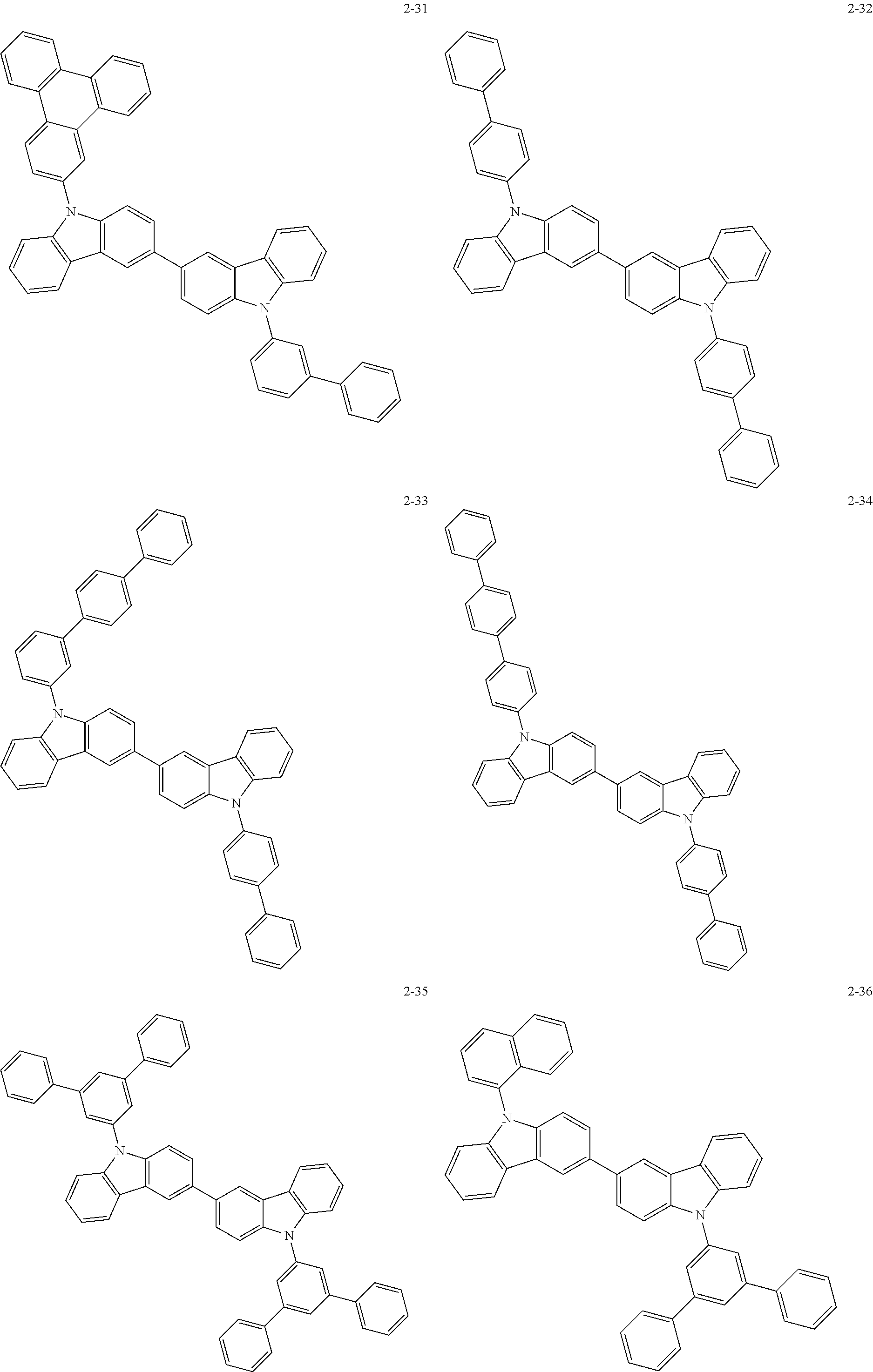

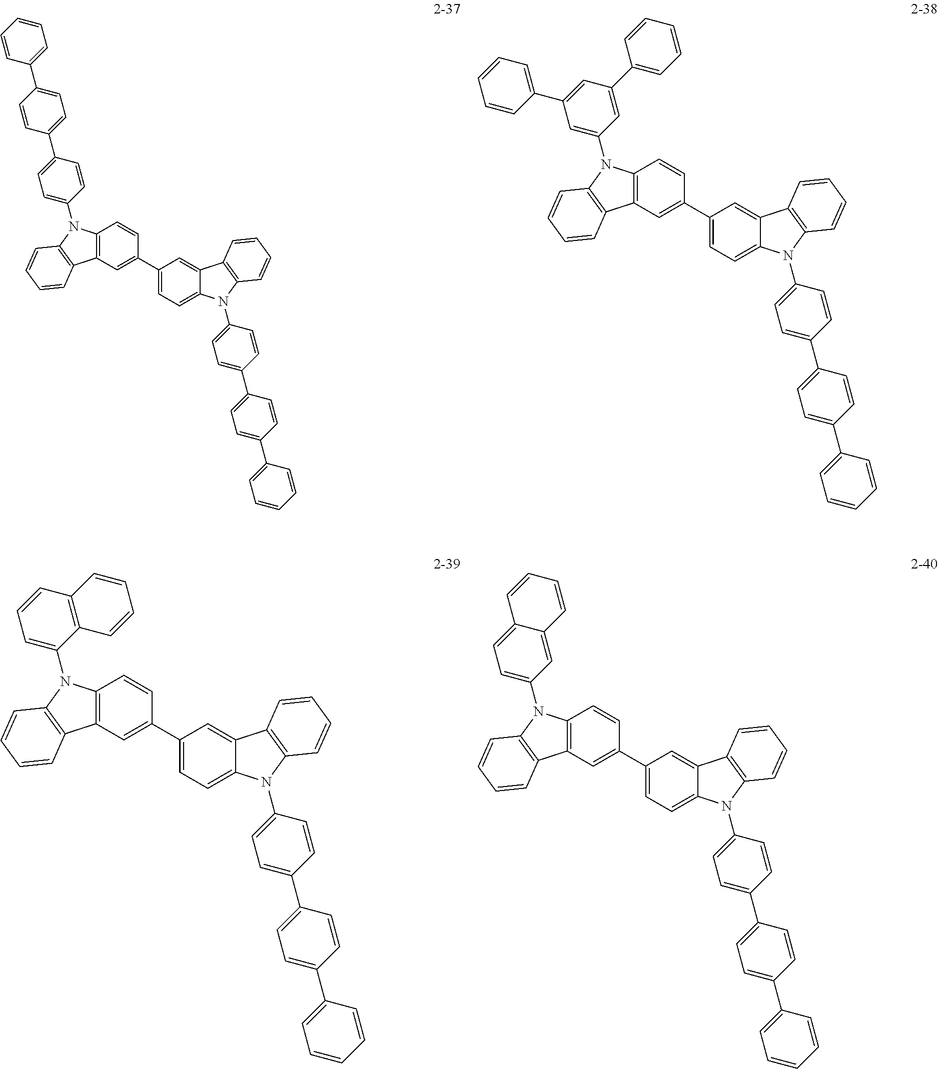 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

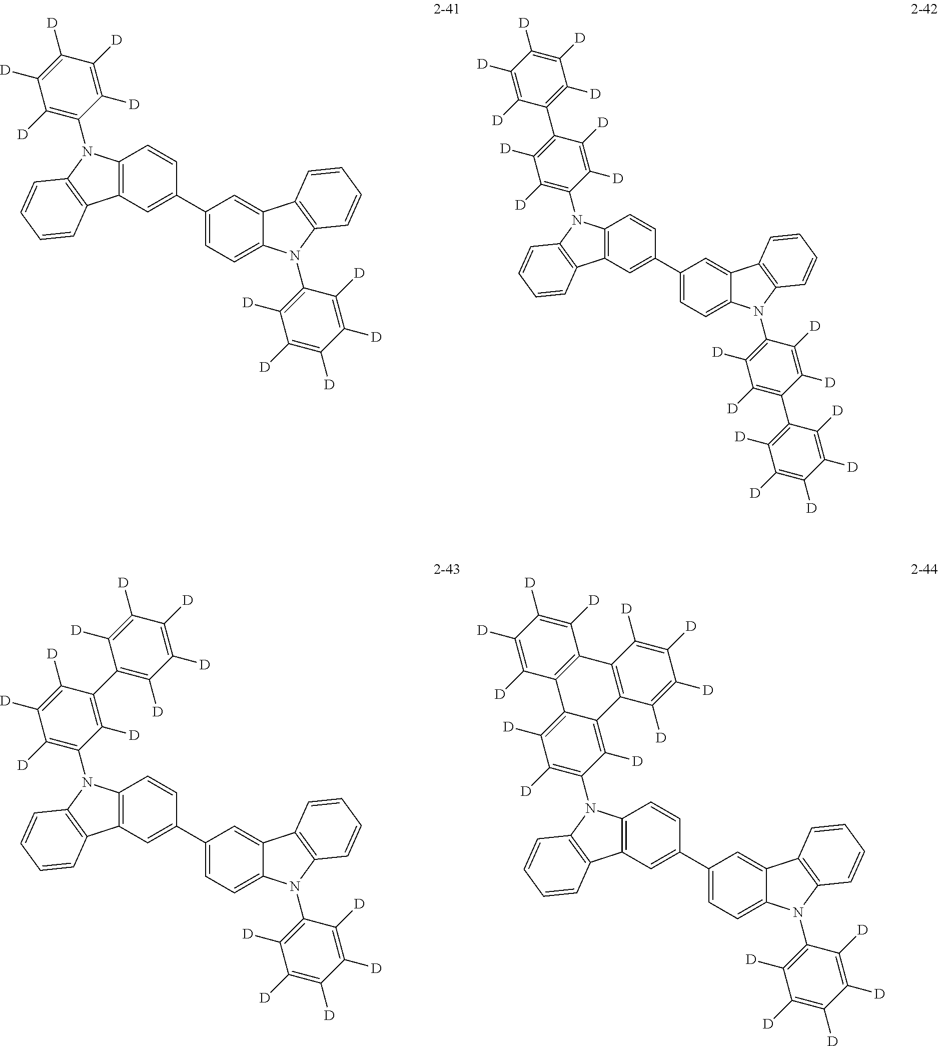

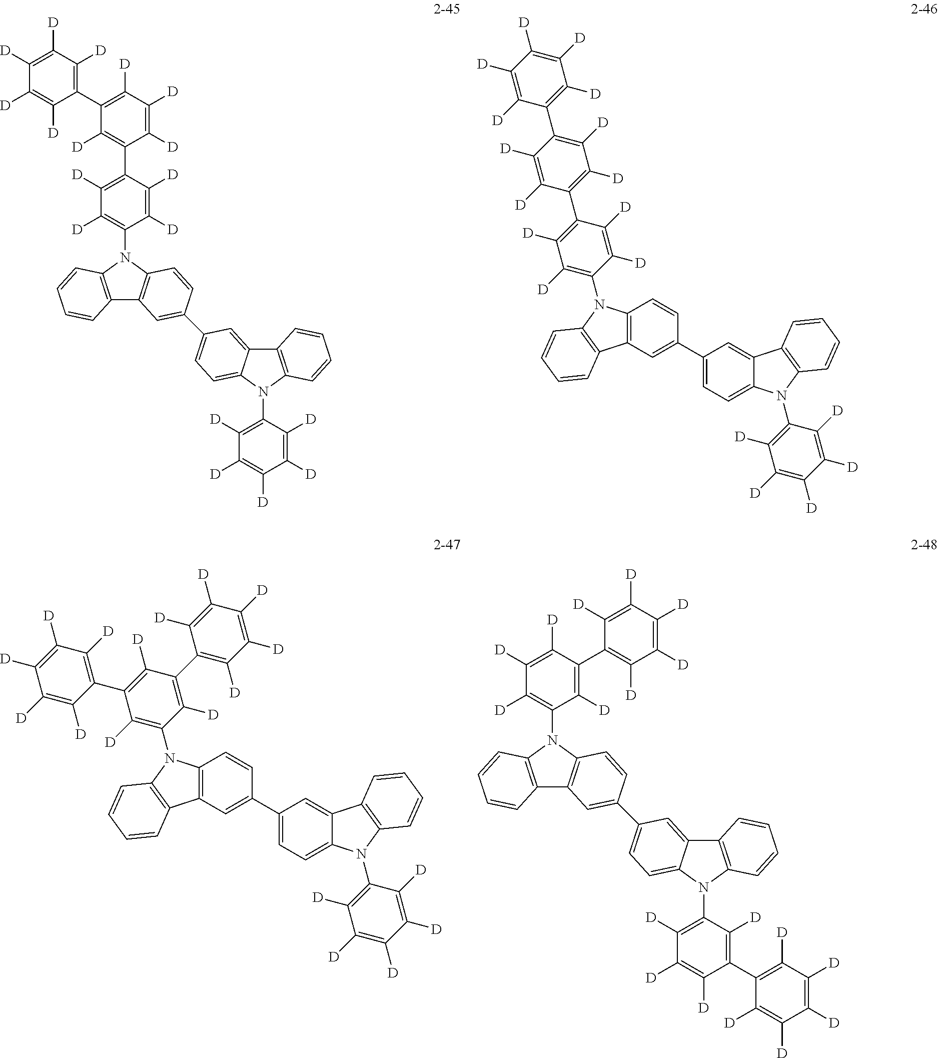

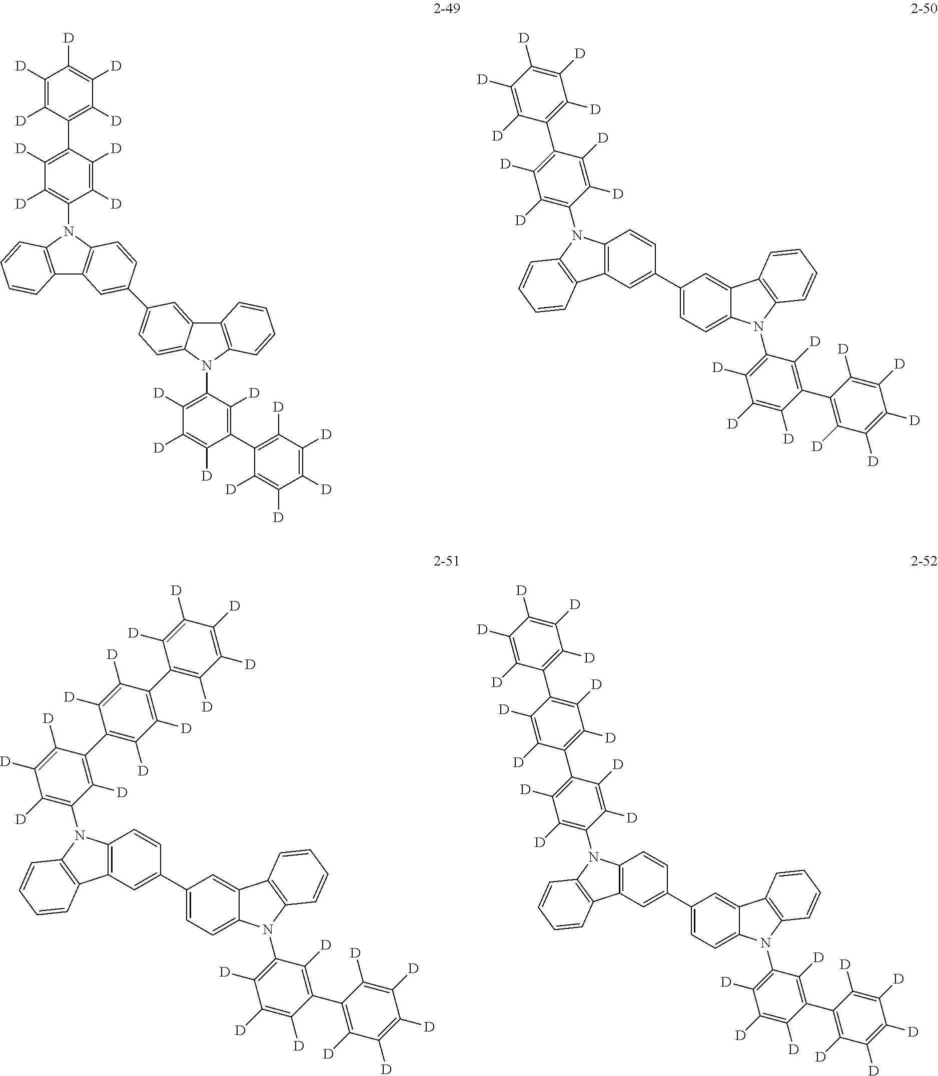

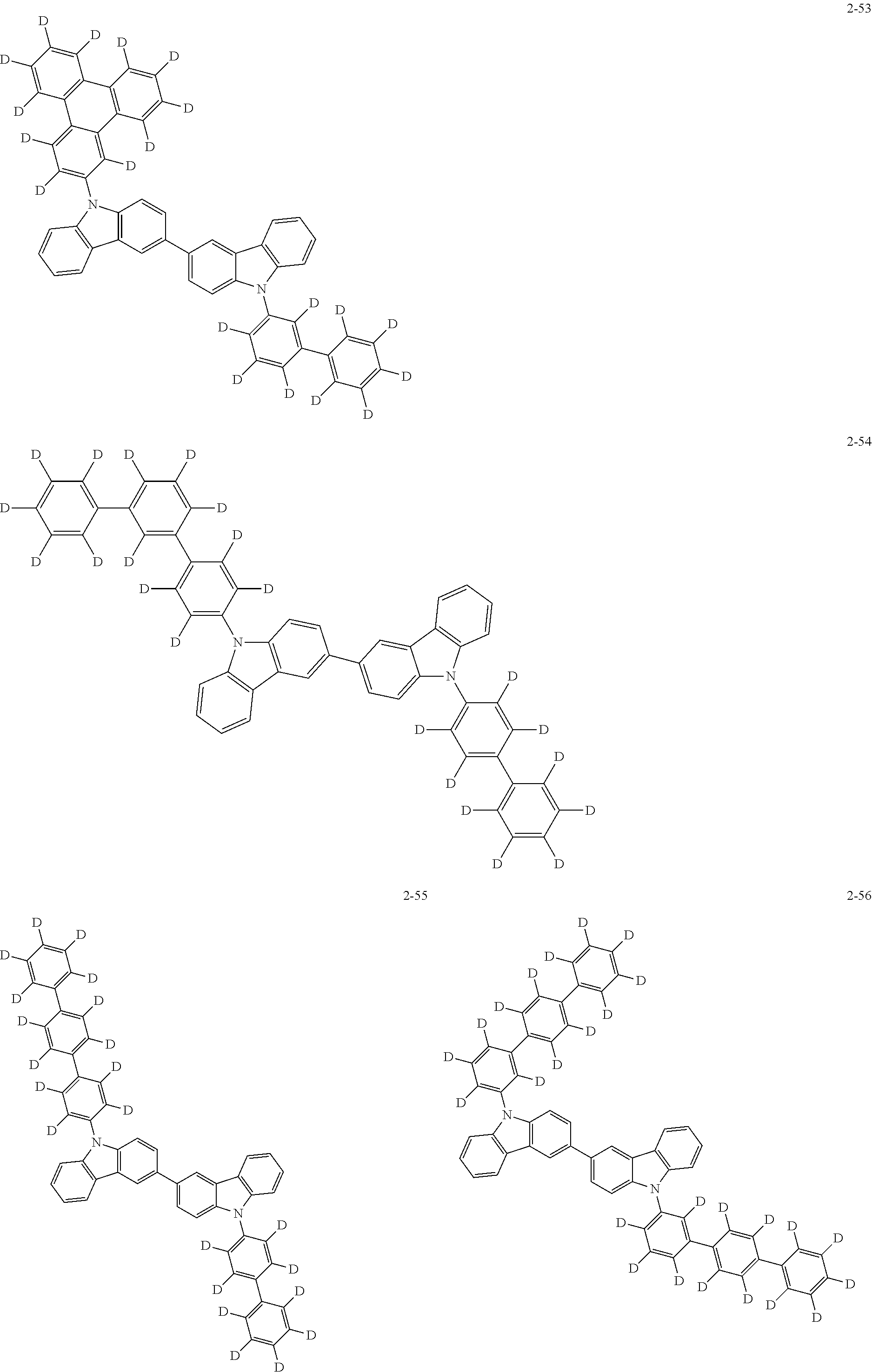

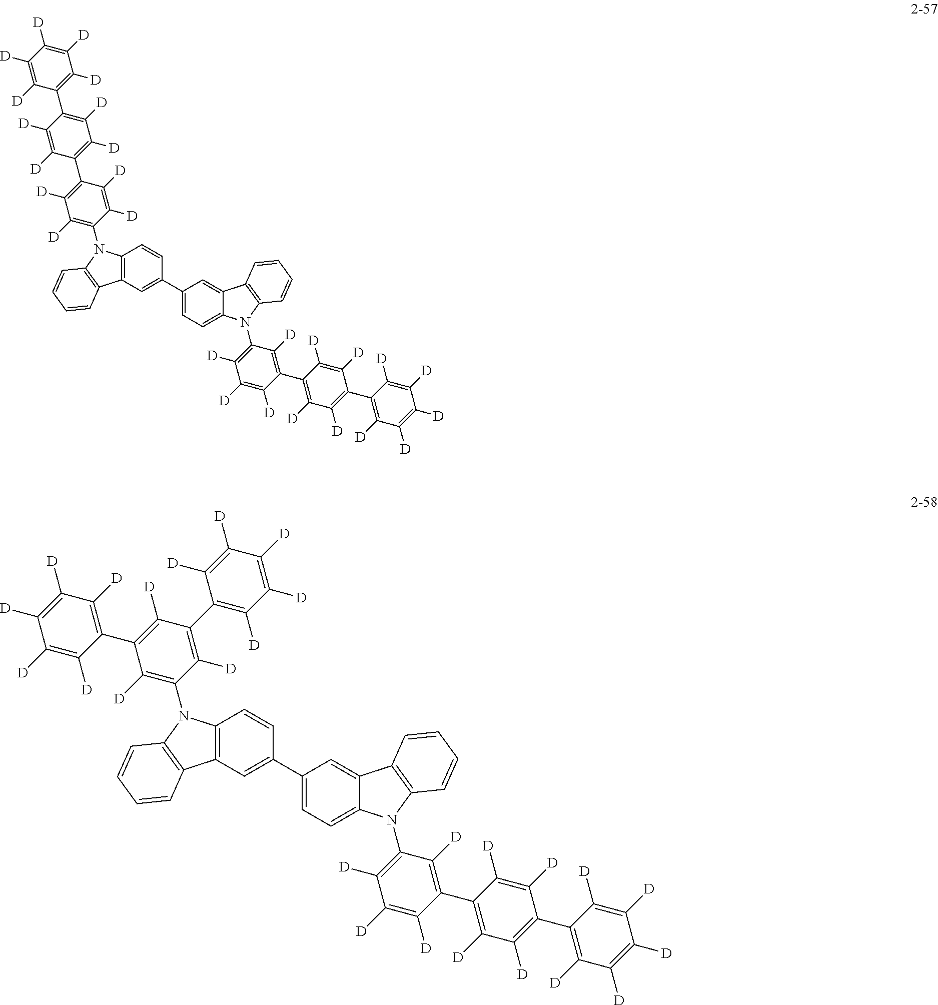

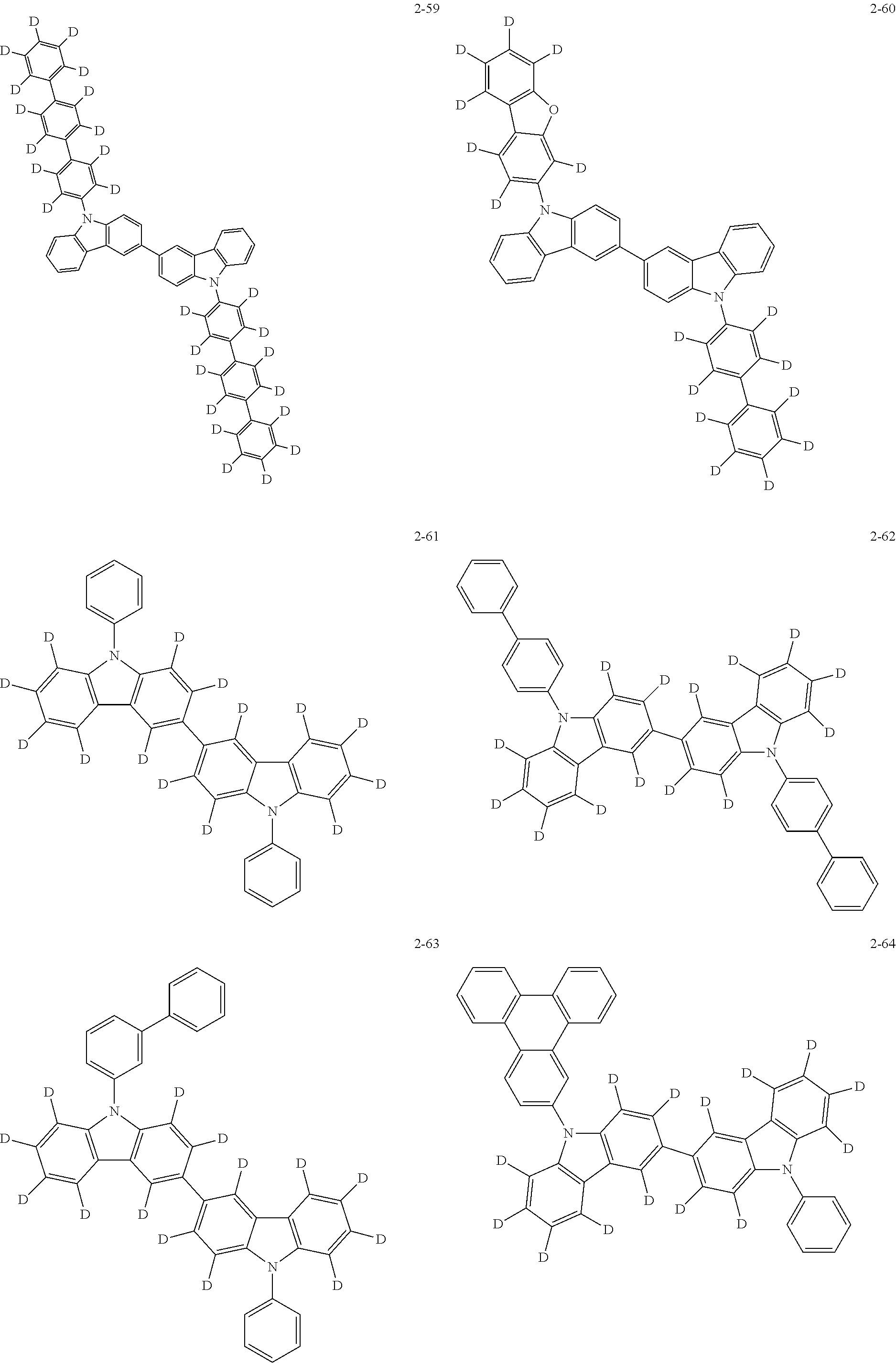

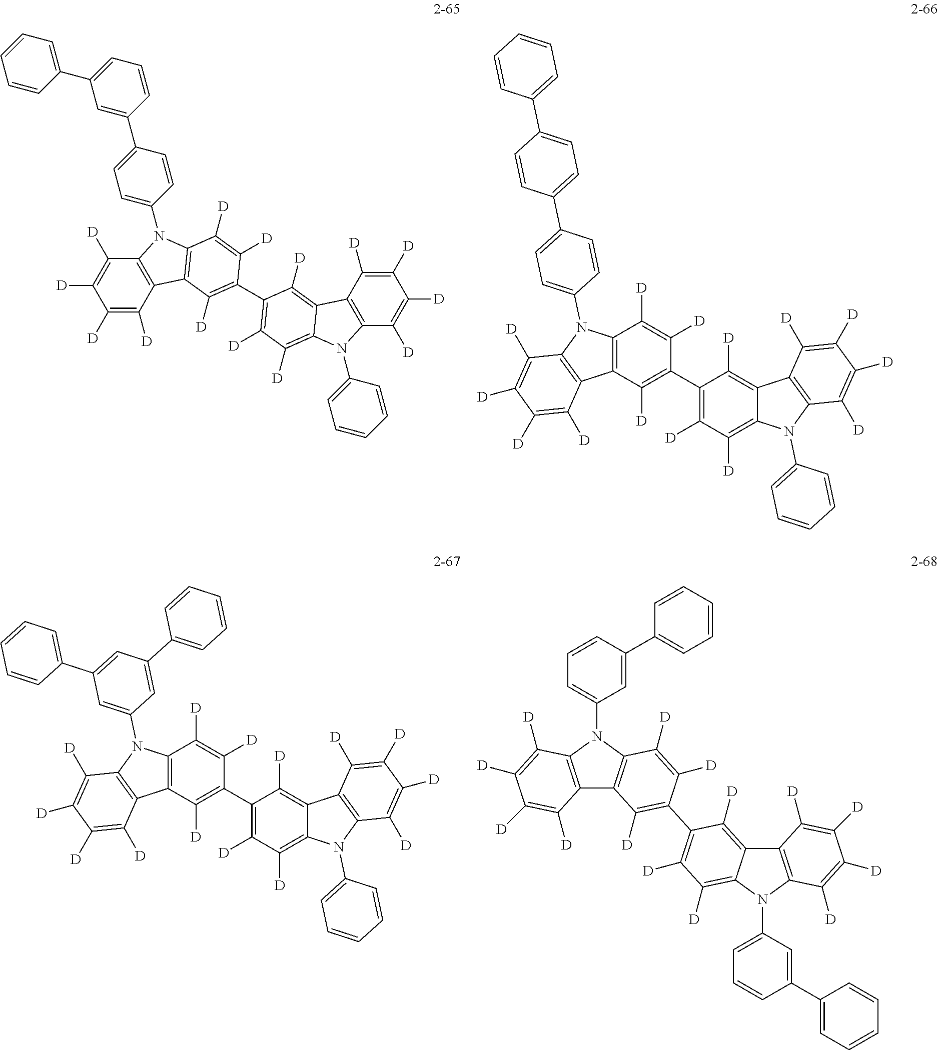

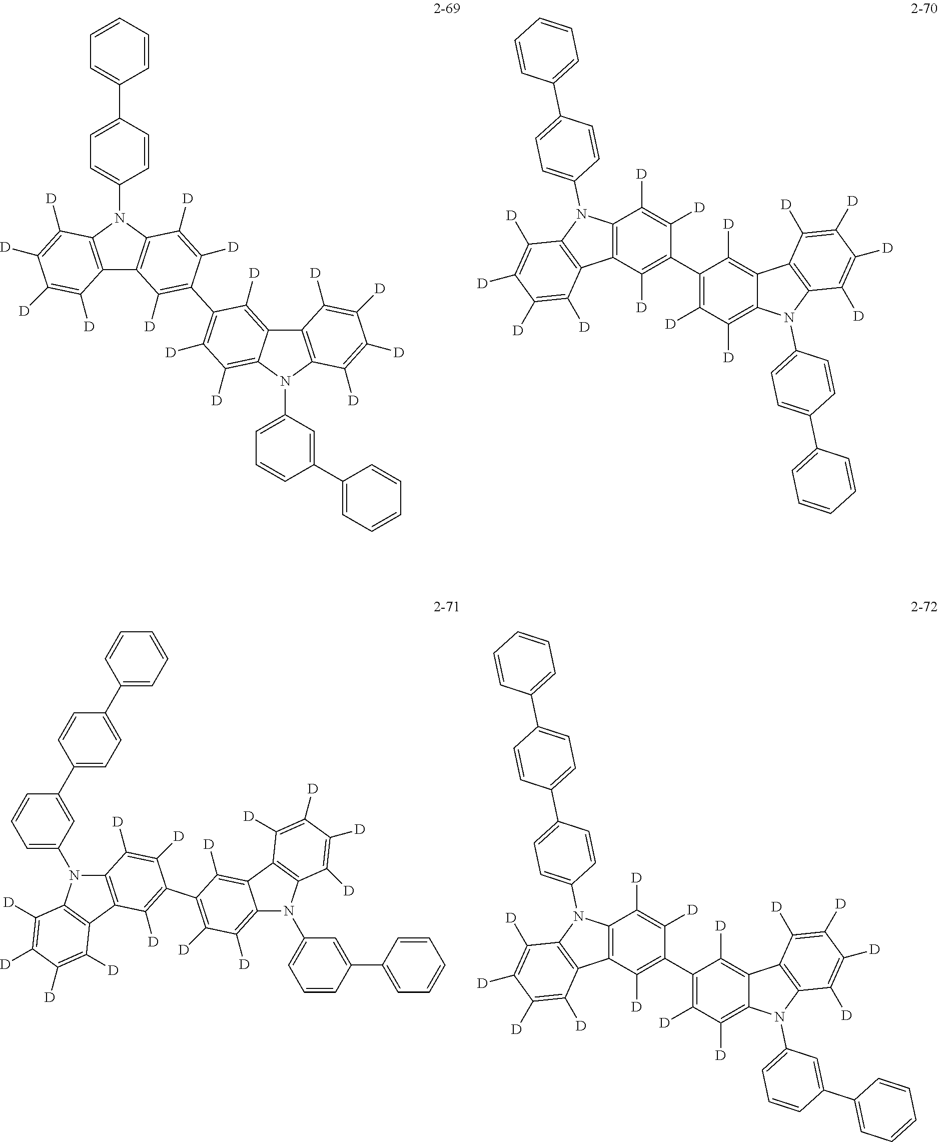

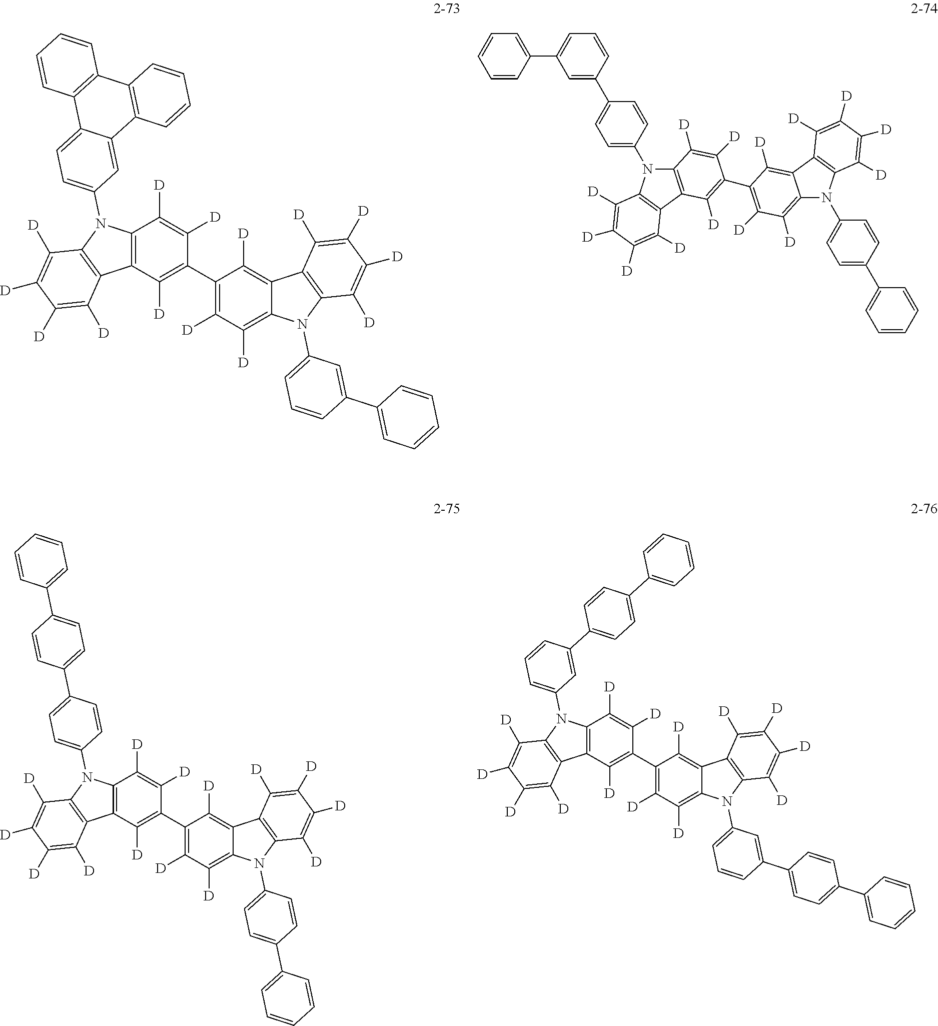

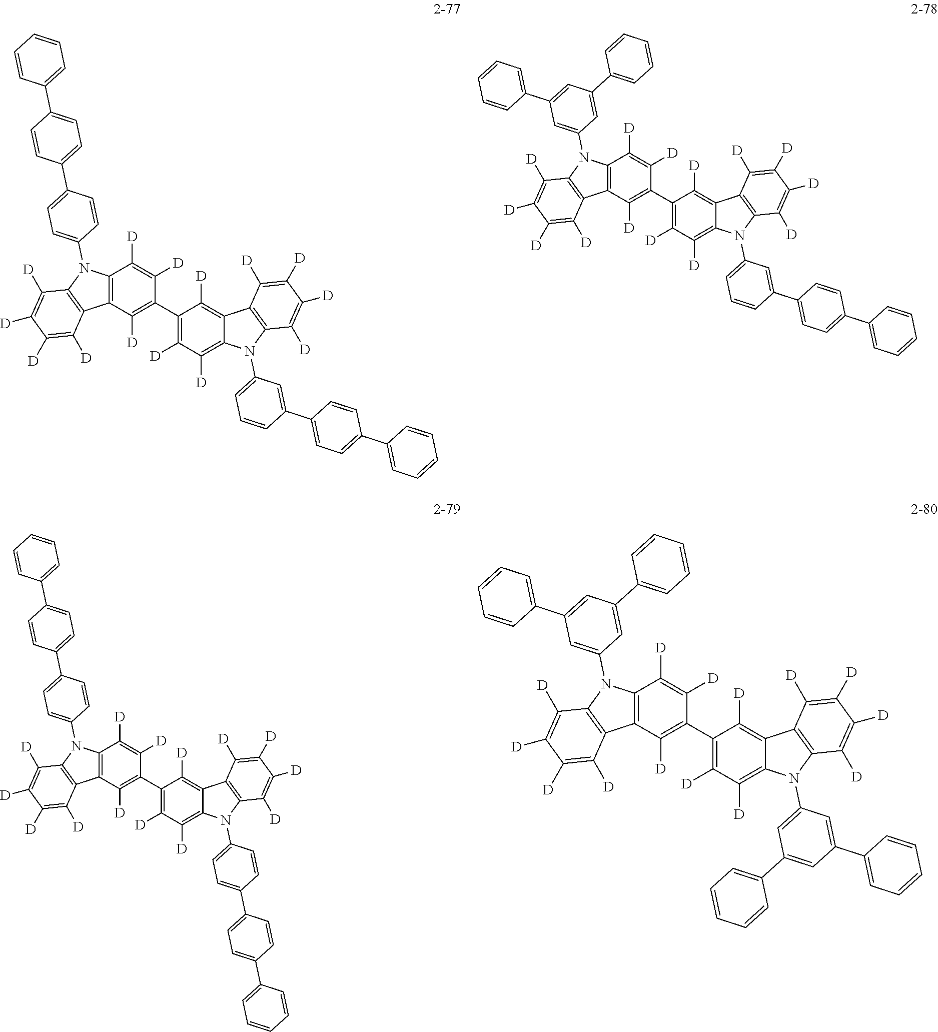

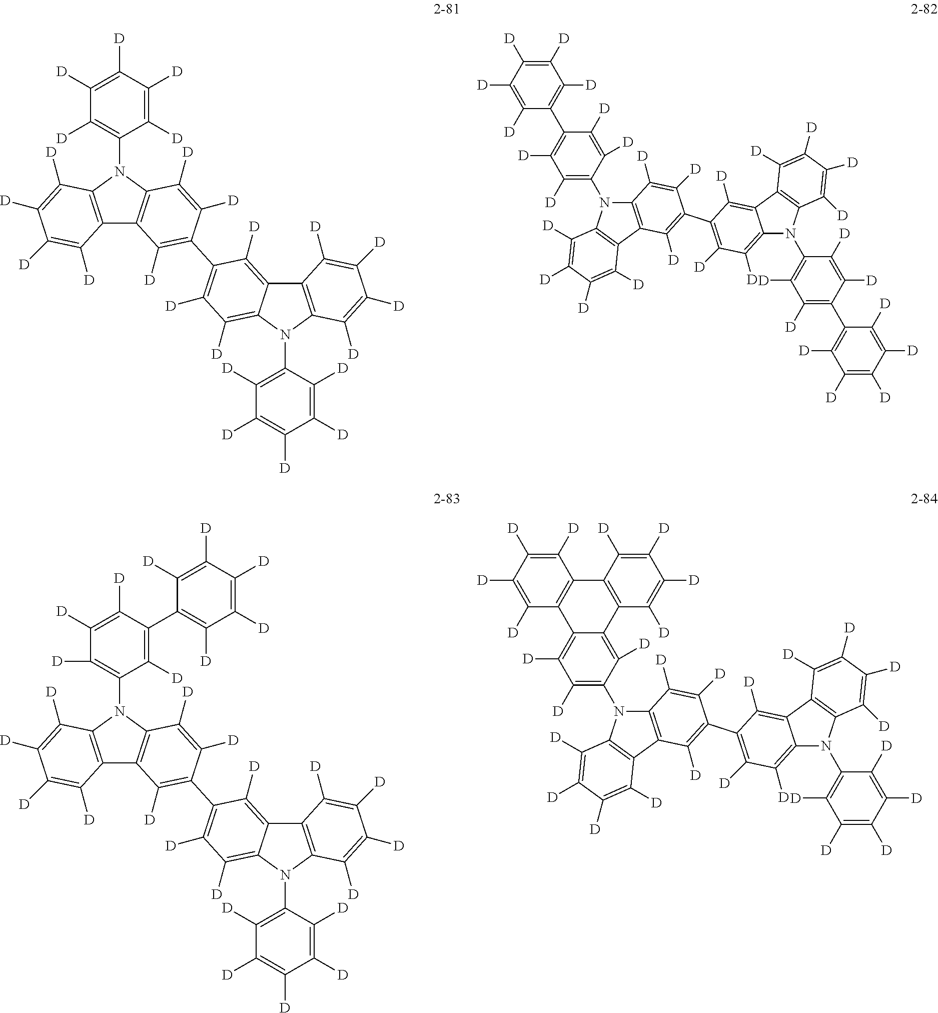

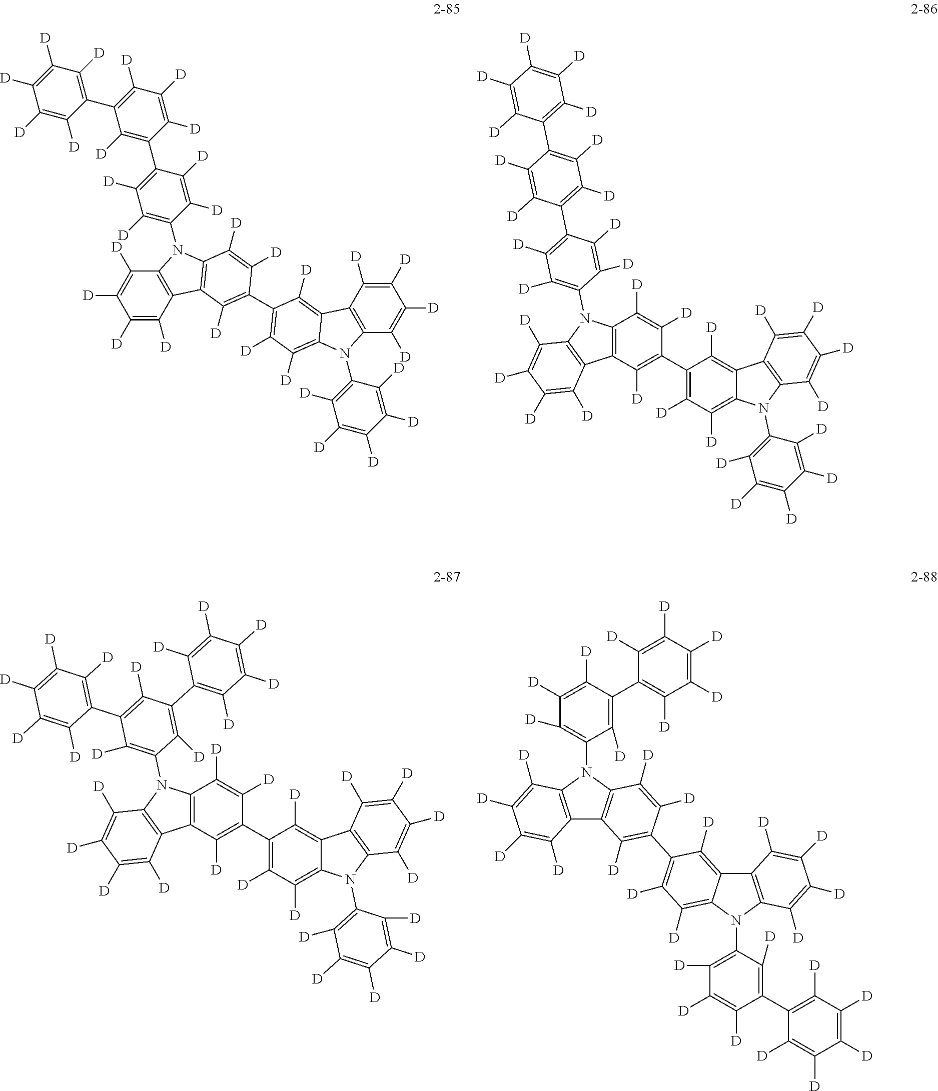

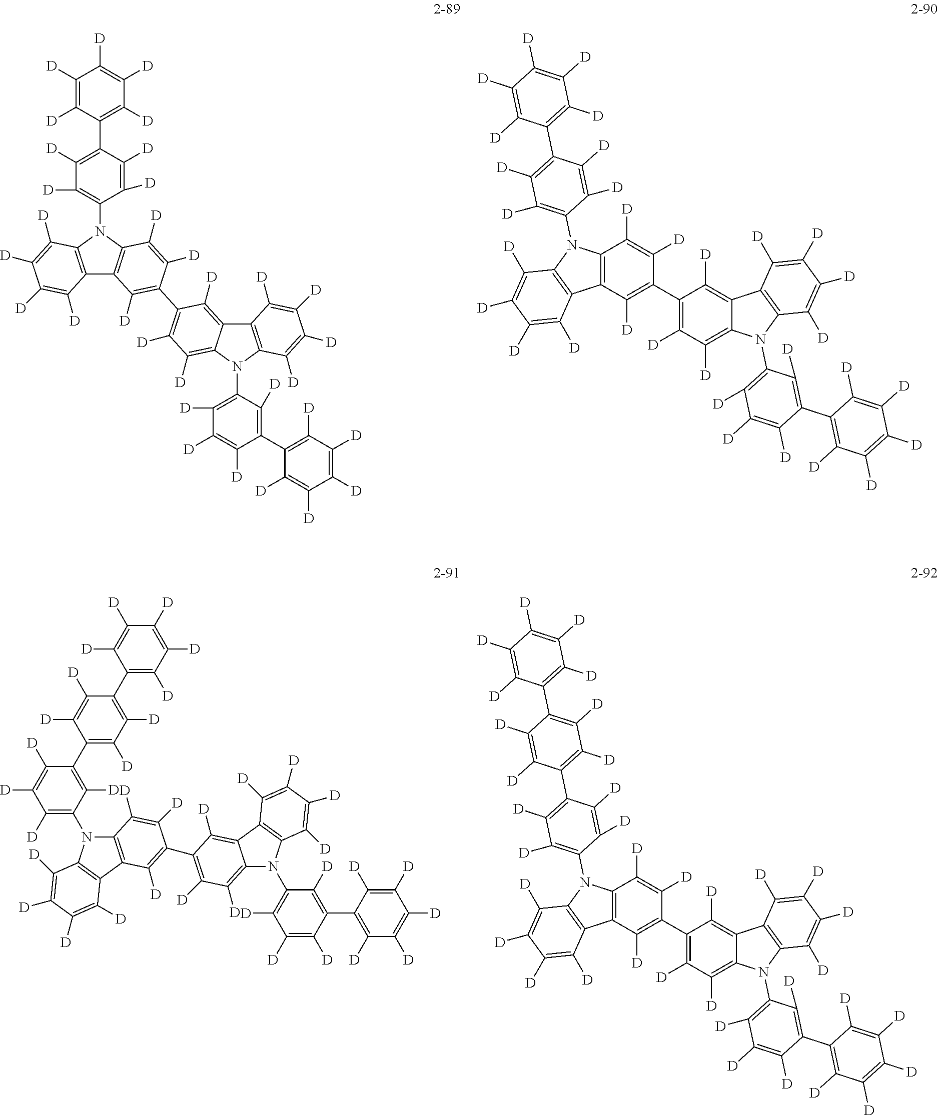

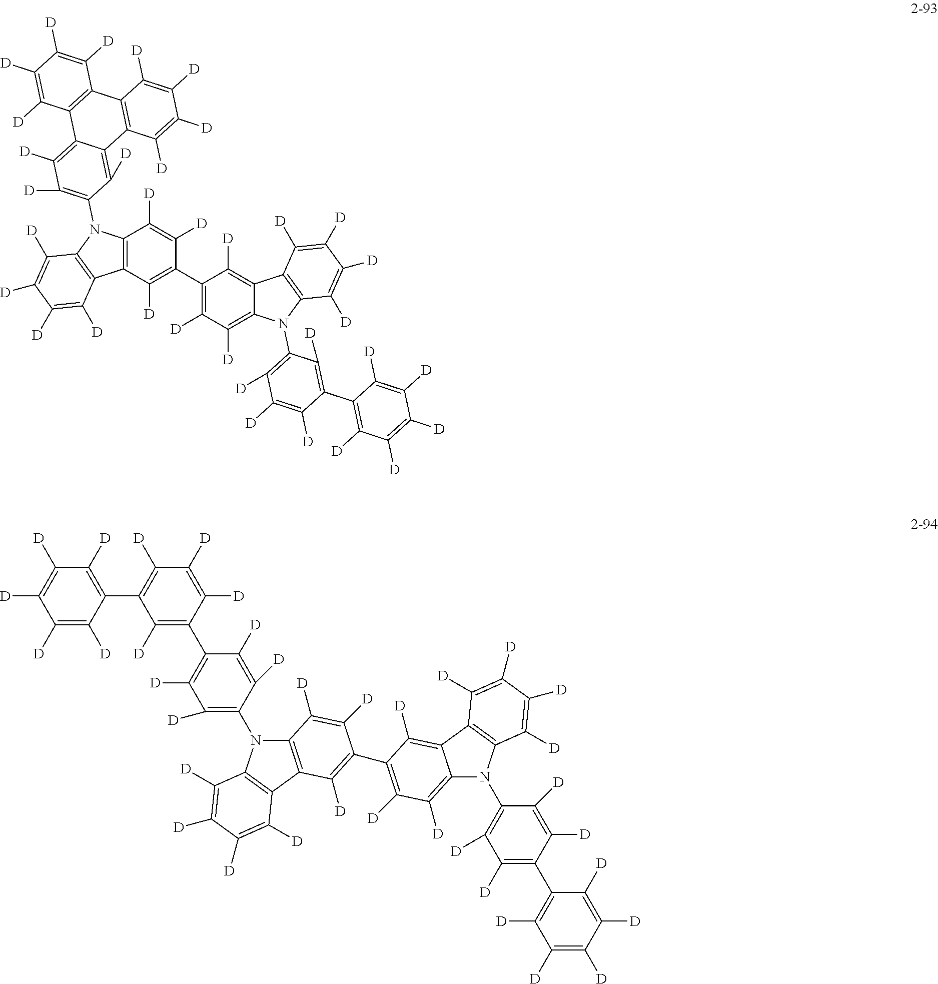

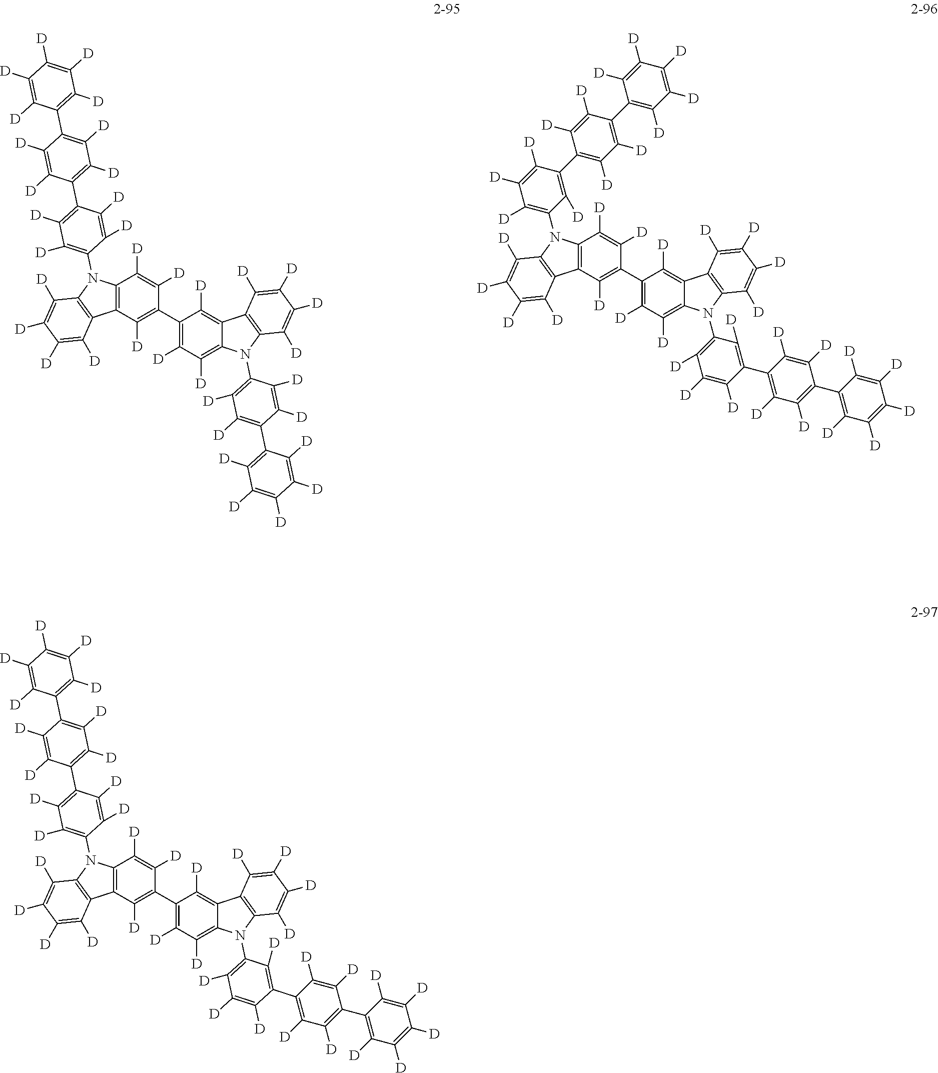

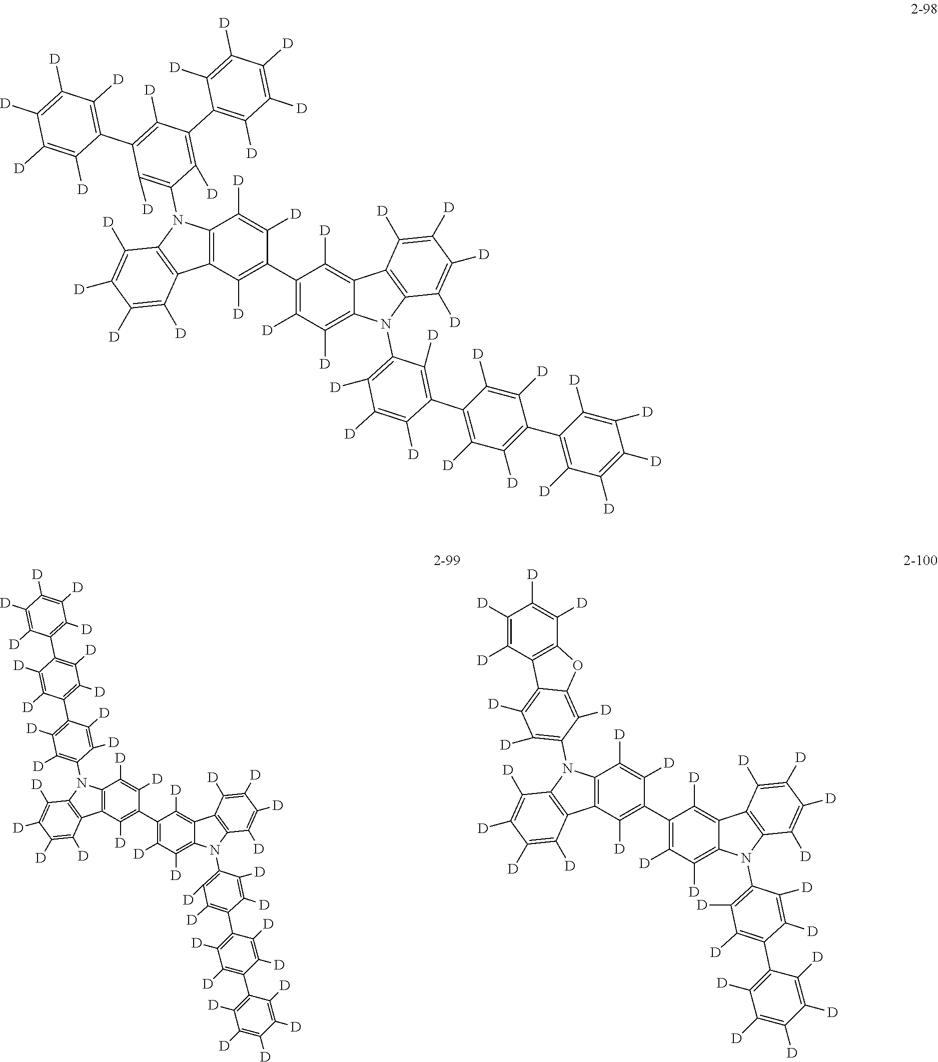

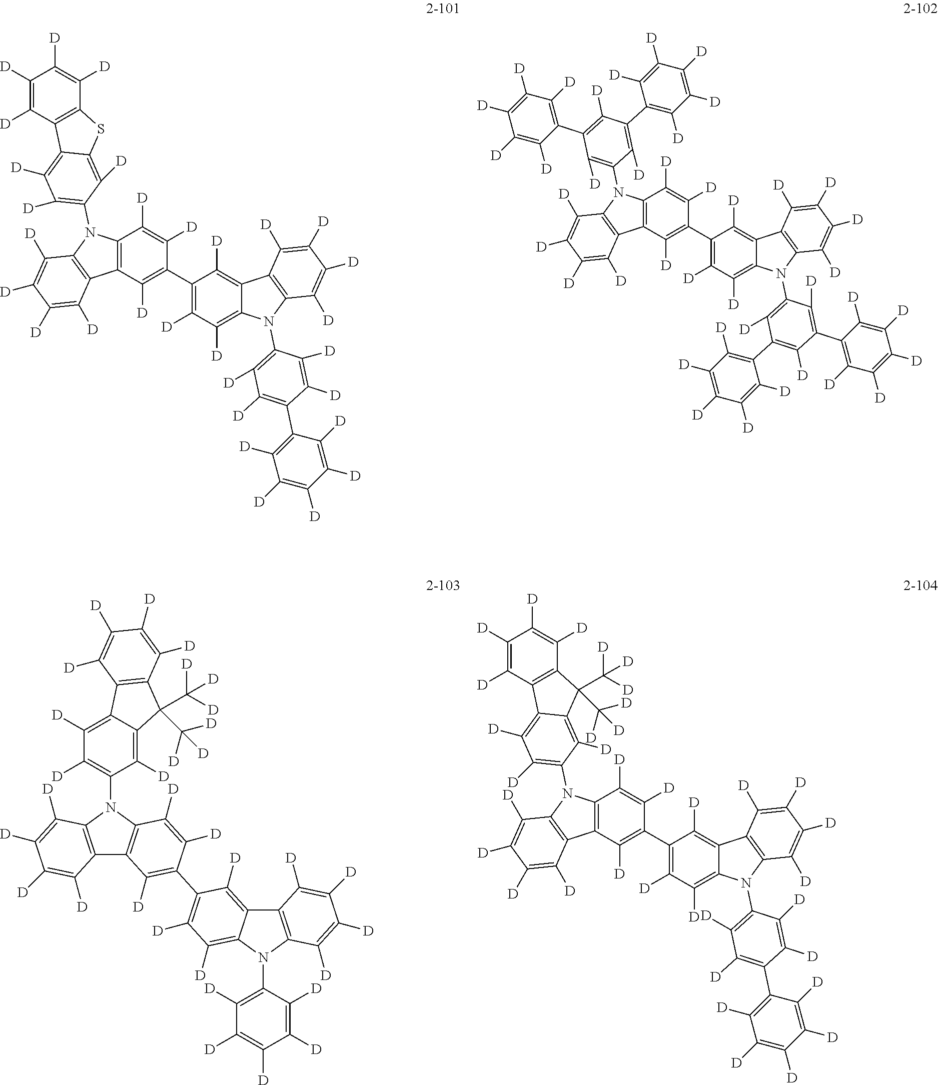

In one embodiment of the present specification, the organic material layer may include the heterocyclic compound of Formula 1 and the compound of Formula 2 at a weight ratio of 1:10 to 10:1, and preferably 1:8 to 8:1, 1:5 to 5:1, or 1:3 to 3:1.

In one embodiment of the present specification, the organic material layer may include the heterocyclic compound of Formula 1 and the compound of Formula 2 at a weight ratio of 1:1 to 1:3.

In one embodiment of the present specification, the organic material layer may further include a phosphorescent dopant.

In one embodiment of the present specification, the phosphorescent dopant may be a green, blue, or red phosphorescent dopant.

In one embodiment of the present specification, the phosphorescent dopant may be a green phosphorescent dopant.

In another embodiment, the phosphorescent dopant may be a red phosphorescent dopant.

In one embodiment of the present specification, the phosphorescent dopant may be an iridium-based dopant.

As a material for the phosphorescent dopant, materials known in the art may be used. For example, a phosphorescent dopant material represented by LL′MX′, LL′L″M, LMX′X″, L₂MX′, or L₃M may be used, but the scope of the present application is not limited by the above examples.

Here, L, L′, L″, X′ and X″ are different bidentate ligands, and M is a metal forming an octahedral complex.
M may be iridium, platinum, or osmium.
L, L′ and L″ are anionic bidentate ligands coordinated to M with the iridium-based dopant by sp2 carbon and a heteroatom, and non-limiting examples of L, L′ and L″ may include 1-phenylisoquinoline, 2-(1-naphthyl)benzoxazole, 2-phenylbenzoxazole, 2-phenylbenzothiazole, 2-phenylbenzothiazole, 7,8-benzoquinoline, thiophene pyrizine, phenylpyridine, benzothiophene pyrizine, 3-methoxy-2-phenylpyridine, thiophene pyridine, and tolylpyridine.
X′ and X″ have a function of trapping electrons or holes, and non-limiting examples of X′ and X″ include acetylacetonate (acac), hexafluoroacetylacetonate, salicylidene, picolinate, and 8-hydroxyquinolinate.

In one embodiment of the present specification, as the iridium-based dopant, a green phosphorescent dopant Ir(ppy)₃ may be used.

In one embodiment of the present specification, as the iridium-based dopant, a red phosphorescent dopant Ir(piq)2(acac) may be used.

In one embodiment of the present specification, the content of the dopant may be 1% to 15%, and preferably 1% to 10% based on the total content of the emissive layer.

The organic light emitting device according to one embodiment of the present specification may be manufactured by conventional methods and materials for manufacturing an organic light emitting device, except that the above-described heterocyclic compound of Formula 1 is used alone, or one or more organic material layers are formed with the compound of Formula 2.

The heterocyclic compound of Formula 1 and the compound of Formula 2 may form an organic material layer by solution coating as well as vacuum deposition in the manufacture of the organic light emitting device. Here, the solution coating includes spin coating, dip coating, inkjet printing, screen printing, spraying, and roll coating, but the present application is not limited thereto.

The organic material layer of the organic light emitting device of the present application may be formed in a monolayer structure, or a multilayer structure in which two or more organic layers are stacked. For example, the organic light emitting device of the present application may have a structure including a hole injection layer, a hole transport layer, an emissive layer, an electron transport layer, and an electron injection layer as organic material layers. However, the structure of the organic light emitting device is not limited, and may include a lower number of organic material layers.

In one embodiment of the present specification, the first electrode may be a positive electrode, and the second electrode may be a negative electrode.

In another embodiment of the present specification, the first electrode may be a negative electrode, and the second electrode may be a positive electrode.

In the organic light emitting device of the present specification, the organic material layer may include an emissive layer, and the emissive layer may include the heterocyclic compound of Formula 1.

In the organic light emitting device of the present specification, the organic material layer may include an emissive layer, and the emissive layer may include the heterocyclic compound of Formula 1 and the compound of Formula 2.

In the organic light emitting device of the present specification, the organic material layer may include an emissive layer, the emissive layer may include a host, and the host may include the heterocyclic compound of Formula 1.

In the organic light emitting device of the present specification, the heterocyclic compound of Formula 1 may be used as a green host or a red host.

In the organic light emitting device of the present specification, the organic material layer may include an emissive layer, the emissive layer may include a host, and the host may include the heterocyclic compound of Formula 1 and the compound of Formula 2.

In the organic light emitting device of the present specification, the compound of Formula 2 may be used as a green host or a red host.

When the heterocyclic compound of Formula 1 and the compound of Formula 2 are combined to form a host, compared with when one host is used, much better efficiency and a much better lifetime are exhibited.

In one embodiment of the present specification, the heterocyclic compound of Formula 1 may be used as an N-type host material, and the compound of Formula 2 may be used as a P-type host material.

In one embodiment of the present specification, the organic light emitting device may be a blue organic light emitting device, and the heterocyclic compound of Formula 1 may be used as a material of the blue organic light emitting device. For example, the heterocyclic compound of Formula 1 may be included in a host material of an emissive layer of the blue organic light emitting device.

In another embodiment of the present specification, the organic light emitting device may be a green organic light emitting device, and the heterocyclic compound of Formula 1 may be used as a material of the green organic light emitting device. For example, the heterocyclic compound of Formula 1 may be included in a host material of an emissive layer of the green organic light emitting device.

In still another embodiment of the present specification, the organic light emitting device may be a red organic light emitting device, and the heterocyclic compound of Formula 1 may be used as a material of the red organic light emitting device. For example, the heterocyclic compound of Formula 1 may be included in a host material of an emissive layer of the red organic light emitting device.

The organic light emitting device of the present application may further include one or more layers selected from the group consisting of an emissive layer, a hole injection layer, a hole transport layer, an electron injection layer, an electron transport layer, an electron blocking layer, and a hole blocking layer.

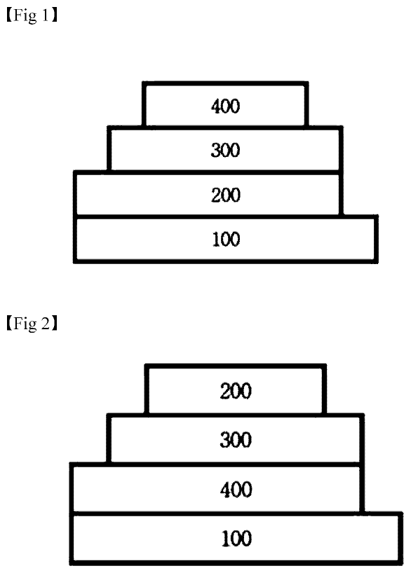

FIGS. 1 to 3 illustrate the stacking orders of electrodes and organic material layers of organic light emitting devices according to an embodiment of the present specification. However, it is not intended that the scope of the present application is limited by these drawings, and the structures of organic light emitting device known in the art may also be applied to the present application.

Referring to FIG. 1, an organic light emitting device in which a positive electrode 200, an organic material layer 300 and a negative electrode 400 are sequentially stacked on a substrate 100 is illustrated. However, the organic light emitting device is not limited to the exemplified structure, and as shown in FIG. 2, an organic light emitting device in which a negative electrode, an organic material layer and a positive electrode are sequentially stacked on a substrate may be implemented.

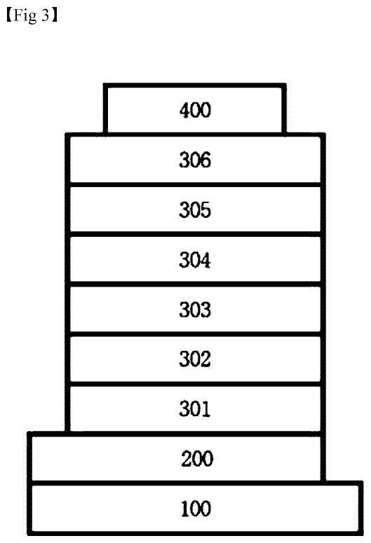

FIG. 3 illustrates that an organic material layer is multilayered. The organic light emitting device shown in FIG. 3 includes a hole injection layer 301, a hole transport layer 302, an emissive layer 303, a hole blocking layer 304, an electron transport layer 305, and an electron injection layer 306. However, the scope of the present application is not limited by the stacked structure, and as needed, layers other than the emissive layer may be omitted, and a necessary functional layer may be further added.

The organic material layer including the heterocyclic compound of Formula 1 may further include another material as needed.

In the organic light emitting device according to one embodiment of the present specification, materials other than the heterocyclic compound of Formula 1 and the compound of Formula 2 are exemplified below. However, these materials are merely provided to exemplify, and not to limit the scope of the present application. The materials may be replaced with materials known in the art.

As a positive electrode material, materials having a relatively large work function may be used, and a transparent conductive oxide, a metal or a conductive polymer may be used. As a specific example of the positive electrode material, a metal such as vanadium, chromium, copper, zinc, or gold, or an alloy thereof; a metal oxide such as zinc oxide, indium oxide, indium tin oxide (ITO), or indium zinc oxide (IZO); a combination of a metal and an oxide, such as ZnO:Al or SnO₂:Sb; or a conductive polymer such as poly(3-methylthiophene), poly[3,4-(ethylene-1,2-dioxy)thiophene] (PEDOT), polypyrrole, or polyaniline may be used, but the present application is not limited thereto.

As a negative electrode material, materials having a relatively low work function, for example, a metal, a metal oxide, or a conductive polymer, may be used. As a specific example of the negative electrode material, a metal such as magnesium, calcium, sodium, potassium, titanium, indium, yttrium, lithium, gadolinium, aluminum, silver, tin, or lead, or an alloy thereof, or a multilayered material such as LiF/Al or LiO₂/Al may be used, but the present application is not limited thereto.

As a hole injection material, a known hole injection material may be used, and for example, a phthalocyanine compound such as copper phthalocyanine, disclosed in U.S. Pat. No. 4,356,429, a starburst-based amine derivatives disclosed in the literature [Advanced Materials, 6, p. 677 (1994)], for example, tris(4-crbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), or a conductive polymer with solubility, such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate) may be used.

As a hole transport material, a pyrazoline derivative, an arylamine-based derivative, a stilbene derivative, or a triphenyl diamine derivative may be used, and a low molecular weight or high molecular weight material may be used.

As an electron transport material, an oxadiazole derivative, anthraquinodimethane and a derivative thereof, benzoquinone and a derivative thereof, naphthoquinone and a derivative thereof, anthraquinone and a derivative thereof, tetracyanoanthratraquinodimethane and a derivative thereof, a fluorenone derivative, diphenyldicyanoethylene and a derivative thereof, a diphenoquinone derivative, or a metal complex of 8-hydroxyquinoline and a derivative thereof may be used, and a low molecular weight material as well as a high molecular weight material may also be used.

As an electron injection material, for example, LiF is representatively used in the art, but the present application is not limited thereto.

As an emissive material, a red, green or blue emissive material may be used, and when needed, two or more emissive materials may be mixed and used. Here, two or more emissive materials may be deposited and used as an individual source, or may be pre-mixed and deposited to be used as one source. In addition, a fluorescent material may be used as an emissive material, but a phosphorescent material may also be used. As an emissive material, although a material that emits light by combining holes and electrons, which are injected from a positive electrode and a negative electrode, respectively, may be used alone, materials in which a host material and a dopant material are involved in light emission may also be used.

When a host of the emissive material is mixed and used, hosts of the same series may be mixed and used, or hosts of different series may be mixed and used For example, any two or more types of materials may be selected from N-type host materials and P-type host materials and used as host materials of the emissive layer.

The organic light emitting device according to one embodiment of the present in specification may be a top emission type, bottom emission type, or dual emission type depending on materials used.

The compound according to one embodiment of the present specification may act on a principle similar to that applied to organic light emitting devices in organic electronic devices including an organic solar cell, an organic photoreceptor, and an organic transistor.

In addition, by introducing various substituents into the structure of Formula 1, it is possible to finely control an energy band gap, improve characteristics at the interface between organic materials, and diversify the use of the material

In another embodiment of the present specification, a composition for an organic material layer of an organic light emitting device including the heterocyclic compound of Formula 1 is provided.

In still another embodiment of the present specification, a composition for an organic material layer of an organic light emitting device including the heterocyclic compound of Formula 1 and the compound of Formula 2 is provided.

Specific details of the heterocyclic compound of Formula 1 and the compound of Formula 2 are the same as described above.

The weight ratio of the heterocyclic compound of Formula 1 and the compound of Formula 2 in the composition may be 1:10 to 10:1, 1:8 to 8:1, 1:5 to 5:1, or 1:3 to 3:1, but the present application is not limited thereto.

In one embodiment of the present specification, the composition is formed by simply mixing two or more compounds, before the formation of an organic material layer of the organic light emitting device, a powder-type material may be mixed, and a compound present in a liquid state at an appropriate temperature or more may be mixed. The composition is in a solid state below the melting point of each material, and may be maintained in a liquid phase by adjusting the temperature.

In one embodiment of the present specification, the composition may be in a form in which the heterocyclic compound of Formula 1 and the compound of Formula 2 are simply mixed.

The composition may further include materials known in the art including a solvent, and an additive.

The composition may be used in the formation of an organic material of the organic light emitting device, and is more preferably used particularly in the formation of a host of the emissive layer.

In one embodiment of the present specification, a method of manufacturing an organic light emitting device, which includes: preparing a substrate; forming a first electrode on the substrate; forming one or more organic material layers on the first electrode; and forming a second electrode on the organic material layer(s) is provided, and here, the forming of one or more organic material layers includes forming one or more organic material layers using a composition for an organic material layer including the heterocyclic compound of Formula 1.

A method of manufacturing an organic light emitting device according to another embodiment may include preparing a substrate; forming a first electrode on the substrate; forming one or more organic material layers on the first electrode; and forming a second electrode on the organic material layer(s), and here, the forming of one or more organic material layers may include forming one or more organic material layers using a composition for an organic material layer, which includes the heterocyclic compound of Formula 1 and the compound of Formula 2, and the forming of one or more organic material layers may be performed by thermal vacuum deposition after pre-mixing the heterocyclic compound of Formula 1 and the compound of Formula 2.

The pre-mixing means mixing the heterocyclic compound of Formula 1 and the compound of Formula 2 and containing the mixture in one source before deposition as an organic material layer.

The pre-mixed material may be referred to as a composition for an organic material layer according to one embodiment of the present specification.

Hereinafter, the present specification is described in further detail with reference to examples, but these examples are provided to exemplify the present application, and are not intended to limit the scope of the present application.

### PREPARATION EXAMPLES

### [Preparation Example 1] Preparation of Compound 1-1

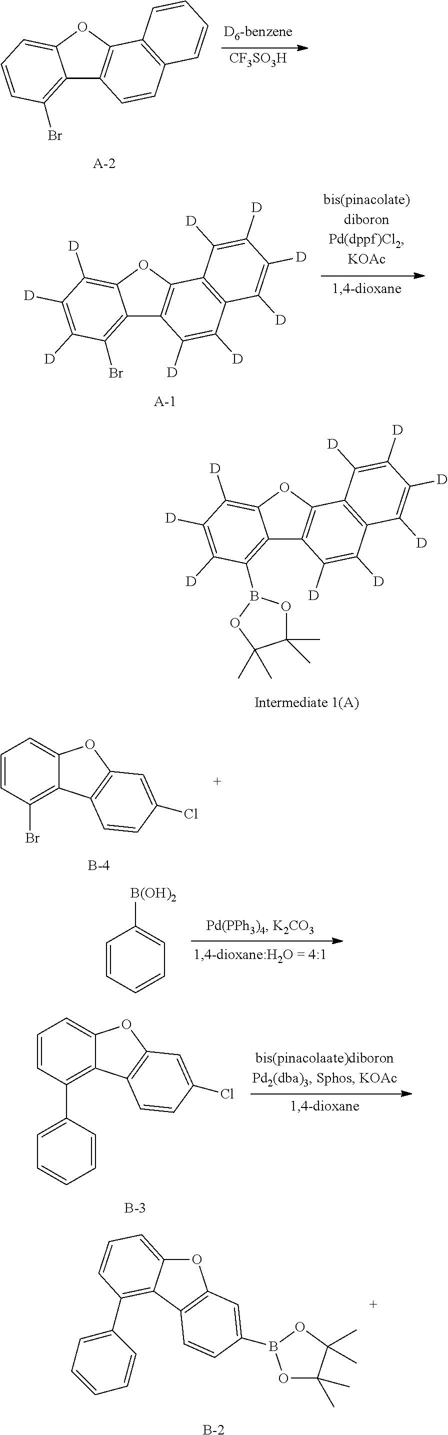

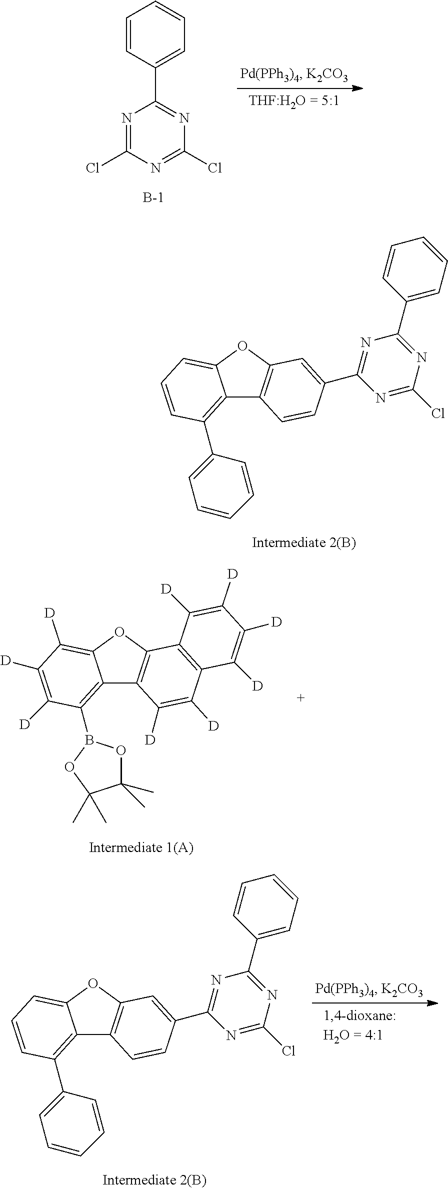

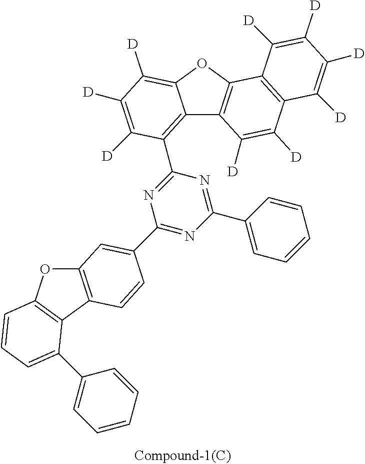 [SMILES: [2H]c1c([2H])c(-c2nc(-c3ccccc3)nc(-c3ccc4c(c3)oc3cccc(-c5ccccc5)c34)n2)c2c(oc3c4c([2H])c([2H])c([2H])c([2H])c4c([2H])c([2H])c32)c1[2H]]

### Synthesis of A-1

7-bromonaphtho[1,2-b]benzofuran (A-2) (10 g, 1 eq.), D6-benzene (500 g, 5941.7 mmol.), and CF₃SO₃H (255 g, 1613 mmol) were put into a reaction flask, and reacted at 50° C. for 1 hour. After completing the reaction, H₂O was added for neutralization, MC and H₂O were added for extraction and an organic layer was purified using a column, thereby obtaining Compound A-1 (8 g, 77%).

### Synthesis of Intermediate 1(A)

A-1 (10 g, 32.6 mmol), bis(pinacolato)diboron (16.6 g, 65.3 mmol), Pd(dppf)Cl₂ (1.2 g, 1.6 mmol), and KOAc (9.6 g, 97.8 mmol) were added to a reaction flask, 100 mL of 1,4-dioxane was added, followed by heating at 120° C. for 4 hours. After completing the reaction, a base was removed, a solvent was concentrated and then column purification was performed, thereby obtaining Intermediate 1(A) (10 g, 87%).

### Synthesis of B-3

1-bromo-7-chlorodibenzo[b,d]furan) (B-4) (15 g, 53.28 mmol), phenylboronic acid (7.79 g, 63.93 mmol), tetrakis(triphenylphosphine)palladium(0) (3.07 g, 2.66 mmol), potassium carbonate (22.09 g, 159.84 mmol), and a 1,4-dioxane/water mixture (150 mL/37.5 mL) were put into a reaction flask and refluxed at 120° C. for 3 hours. After completing the reaction, a solid produced after lowering the temperature to room temperature was washed with distilled water and MeOH, thereby obtaining B-3 (14 g, 94%).

### Synthesis of B-2

B-3 (14 g, 62 mmol), bis(pinacolato)diboron (25.5 g, 100 mmol), Sphos (4.1 g, 10 mmol), KOAc (14.7 g, 150 mmol), and Pd₂(dba)₃ (4.5 g, 5 mmol) were put into a reaction flask, 140 mL of 1,4-dioxane was added, and then the resulting product was heated at 120° C. for 4 hours. After completing the reaction, a solvent was concentrated and then column purification was performed, thereby obtaining B-2 (16 g, 86%).

### Synthesis of Intermediate 2(B)

2,4-dichloro-6-phenyl-1,3,5-triazine (B-1) (6.1 g, 27 mmol), B-2 (10 g, 27 mmol), Pd(PPh₃)₄ (1.6 g, 1.4 mmol), and K₂CO₃ (11.2 g, 81 mmol) were put into a reaction flask, and a THF/water mixture (100 mL/20 mL) were put into a reaction flask, and the reaction product was heated at 85° C. for 4 hours. After completing the reaction, a solid produced after lowering the temperature to room temperature was washed with distilled water and MeOH, thereby obtaining Intermediate 2(B) (9 g, 75%).

### Synthesis of Compound 1-1(C)

Intermediate 1(A)(10 g, 28.3 mmol), Intermediate 2(B) (14.8 g, 34 mmol), Pd(PPh₃)₄ (1.6 g, 1.4 mmol), K₂CO₃ (11.7 g, 84.9 mmol), and a 1,4-dioxane/water mixture (100 mL/25 mL) were added to a reaction flask and refluxed at 120° C. for 3 hours. After completing the reaction, a solid produced after lowering the temperature to room temperature was washed with distilled water and MeOH, thereby obtaining desired Compound 1-1(C) (14 g, 79%).

In Preparation Example 1, desired Compound (C) was synthesized in the same manner as described above, except that Reagents A-2, B-4 and B-1 in Table 1 below were used.

TABLE 1

| Com- | | |
|---|---|---|
| pound | A-2 | B-4 |
| 1-2 | 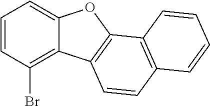 [SMILES: Brc1cccc2oc3c4ccccc4ccc3c12] | 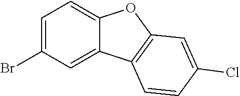 [SMILES: Clc1ccc2c(c1)oc1ccc(Br)cc12] |
| 1-3 | 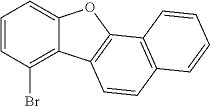 [SMILES: Brc1cccc2oc3c4ccccc4ccc3c12] | 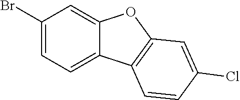 [SMILES: Clc1ccc2c(c1)oc1cc(Br)ccc12] |
| 1-4 | 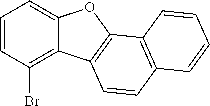 [SMILES: Brc1cccc2oc3c4ccccc4ccc3c12] | 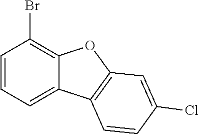 [SMILES: Clc1ccc2c(c1)oc1c(Br)cccc12] |
| 1-5 | 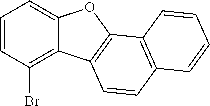 [SMILES: Brc1cccc2oc3c4ccccc4ccc3c12] | 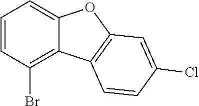 [SMILES: Clc1ccc2c(c1)oc1cccc(Br)c12] |
| 1-6 | 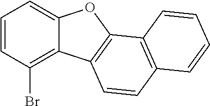 [SMILES: Brc1cccc2oc3c4ccccc4ccc3c12] | 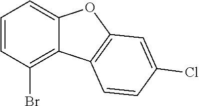 [SMILES: Clc1ccc2c(c1)oc1cccc(Br)c12] |
| 1-7 | 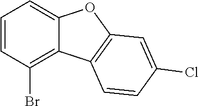 [SMILES: Clc1ccc2c(c1)oc1cccc(Br)c12] | 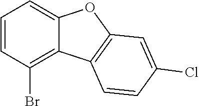 [SMILES: Clc1ccc2c(c1)oc1cccc(Br)c12] |
| 1-8 | 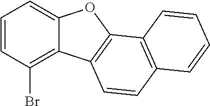 [SMILES: Brc1cccc2oc3c4ccccc4ccc3c12] | 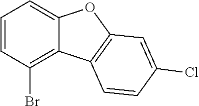 [SMILES: Clc1ccc2c(c1)oc1cccc(Br)c12] |
| 1-9 | 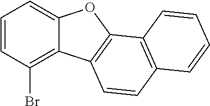 [SMILES: Brc1cccc2oc3c4ccccc4ccc3c12] | 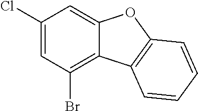 [SMILES: Clc1cc(Br)c2c(c1)oc1ccccc12] |
| 1-10 | 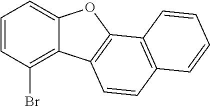 [SMILES: Brc1cccc2oc3c4ccccc4ccc3c12] | 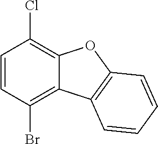 [SMILES: Clc1ccc(Br)c2c1oc1ccccc12] |
| 1-11 | 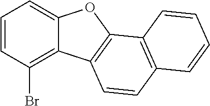 [SMILES: Brc1cccc2oc3c4ccccc4ccc3c12] | 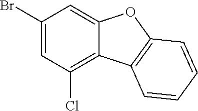 [SMILES: Clc1cc(Br)cc2oc3ccccc3c12] |
| 1-12 | 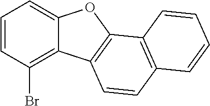 [SMILES: Brc1cccc2oc3c4ccccc4ccc3c12] | 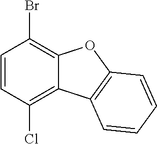 [SMILES: Clc1ccc(Br)c2oc3ccccc3c12] |
| 1-21 | 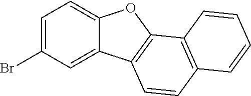 [SMILES: Brc1ccc2oc3c4ccccc4ccc3c2c1] | 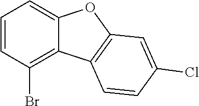 [SMILES: Clc1ccc2c(c1)oc1cccc(Br)c12] |
| 1-22 | 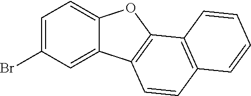 [SMILES: Brc1ccc2oc3c4ccccc4ccc3c2c1] | 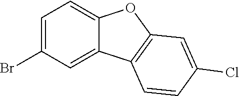 [SMILES: Clc1ccc2c(c1)oc1ccc(Br)cc12] |
| 1-23 | 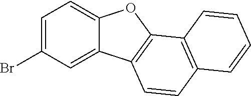 [SMILES: Brc1ccc2oc3c4ccccc4ccc3c2c1] | 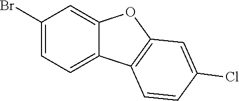 [SMILES: Clc1ccc2c(c1)oc1cc(Br)ccc12] |
| 1-24 | 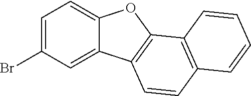 [SMILES: Brc1ccc2oc3c4ccccc4ccc3c2c1] | 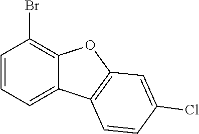 [SMILES: Clc1ccc2c(c1)oc1c(Br)cccc12] |
| 1-25 | 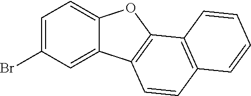 [SMILES: Brc1ccc2oc3c4ccccc4ccc3c2c1] | 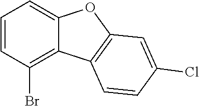 [SMILES: Clc1ccc2c(c1)oc1cccc(Br)c12] |
| 1-26 | 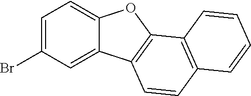 [SMILES: Brc1ccc2oc3c4ccccc4ccc3c2c1] | 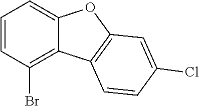 [SMILES: Clc1ccc2c(c1)oc1cccc(Br)c12] |
| 1-27 | 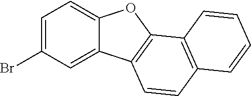 [SMILES: Brc1ccc2oc3c4ccccc4ccc3c2c1] | 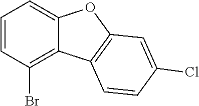 [SMILES: Clc1ccc2c(c1)oc1cccc(Br)c12] |
| 1-28 | 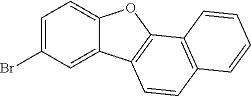 [SMILES: Brc1ccc2oc3c4ccccc4ccc3c2c1] | 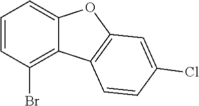 [SMILES: Clc1ccc2c(c1)oc1cccc(Br)c12] |
| 1-29 | 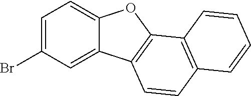 [SMILES: Brc1ccc2oc3c4ccccc4ccc3c2c1] | 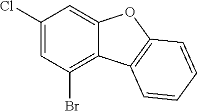 [SMILES: Clc1cc(Br)c2c(c1)oc1ccccc12] |
| 1-30 | 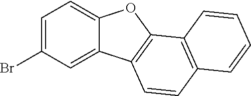 [SMILES: Brc1ccc2oc3c4ccccc4ccc3c2c1] | 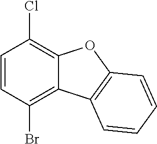 [SMILES: Clc1ccc(Br)c2c1oc1ccccc12] |
| 1-31 | 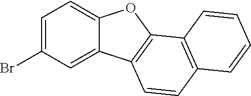 [SMILES: Brc1ccc2oc3c4ccccc4ccc3c2c1] | 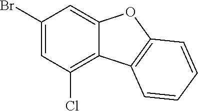 [SMILES: Clc1cc(Br)cc2oc3ccccc3c12] |
| 1-32 | 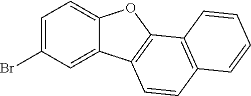 [SMILES: Brc1ccc2oc3c4ccccc4ccc3c2c1] | 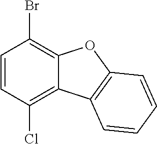 [SMILES: Clc1ccc(Br)c2oc3ccccc3c12] |
| 1-41 | 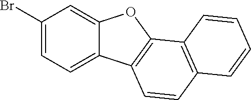 [SMILES: Brc1ccc2c(c1)oc1c3ccccc3ccc21] | 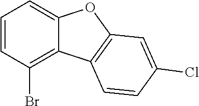 [SMILES: Clc1ccc2c(c1)oc1cccc(Br)c12] |
| 1-42 | 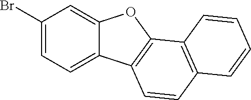 [SMILES: Brc1ccc2c(c1)oc1c3ccccc3ccc21] | 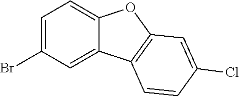 [SMILES: Clc1ccc2c(c1)oc1ccc(Br)cc12] |
| 1-43 | 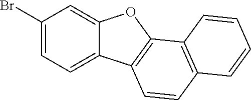 [SMILES: Brc1ccc2c(c1)oc1c3ccccc3ccc21] | 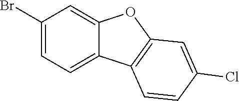 [SMILES: Clc1ccc2c(c1)oc1cc(Br)ccc12] |
| 1-44 | 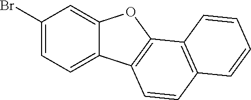 [SMILES: Brc1ccc2c(c1)oc1c3ccccc3ccc21] | 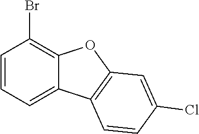 [SMILES: Clc1ccc2c(c1)oc1c(Br)cccc12] |
| 1-45 | 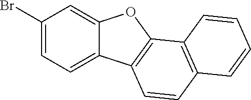 [SMILES: Brc1ccc2c(c1)oc1c3ccccc3ccc21] | 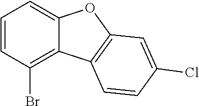 [SMILES: Clc1ccc2c(c1)oc1cccc(Br)c12] |
| 1-46 | 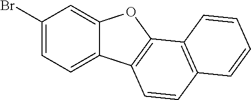 [SMILES: Brc1ccc2c(c1)oc1c3ccccc3ccc21] | 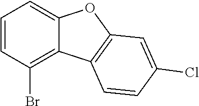 [SMILES: Clc1ccc2c(c1)oc1cccc(Br)c12] |
| 1-47 | 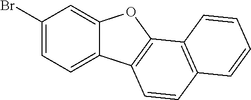 [SMILES: Brc1ccc2c(c1)oc1c3ccccc3ccc21] | 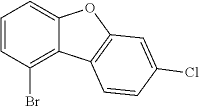 [SMILES: Clc1ccc2c(c1)oc1cccc(Br)c12] |
| 1-48 | 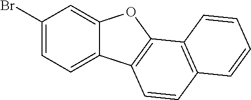 [SMILES: Brc1ccc2c(c1)oc1c3ccccc3ccc21] | 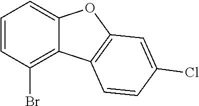 [SMILES: Clc1ccc2c(c1)oc1cccc(Br)c12] |
| 1-49 | 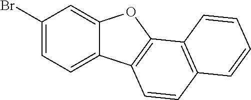 [SMILES: Brc1ccc2c(c1)oc1c3ccccc3ccc21] | 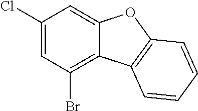 [SMILES: Clc1cc(Br)c2c(c1)oc1ccccc12] |
| 1-50 | 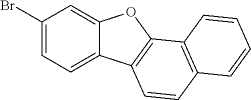 [SMILES: Brc1ccc2c(c1)oc1c3ccccc3ccc21] | 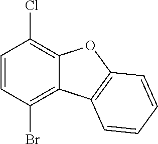 [SMILES: Clc1ccc(Br)c2c1oc1ccccc12] |
| 1-51 | 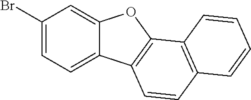 [SMILES: Brc1ccc2c(c1)oc1c3ccccc3ccc21] | 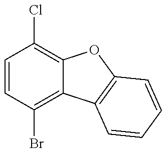 [SMILES: Clc1ccc(Br)c2c1oc1ccccc12] |
| 1-52 | 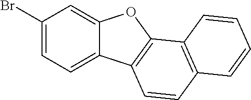 [SMILES: Brc1ccc2c(c1)oc1c3ccccc3ccc21] | 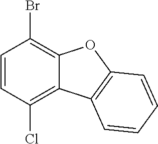 [SMILES: Clc1ccc(Br)c2oc3ccccc3c12] |
| 1-61 | 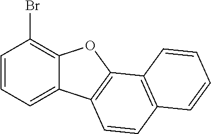 [SMILES: Brc1cccc2c1oc1c3ccccc3ccc21] | 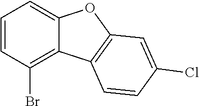 [SMILES: Clc1ccc2c(c1)oc1cccc(Br)c12] |
| 1-62 | 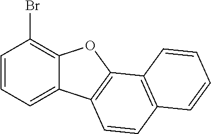 [SMILES: Brc1cccc2c1oc1c3ccccc3ccc21] | 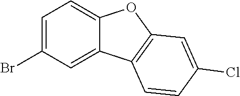 [SMILES: Clc1ccc2c(c1)oc1ccc(Br)cc12] |
| 1-63 | 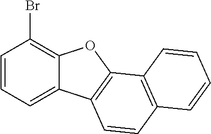 [SMILES: Brc1cccc2c1oc1c3ccccc3ccc21] | 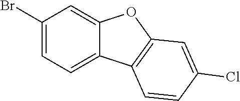 [SMILES: Clc1ccc2c(c1)oc1cc(Br)ccc12] |
| 1-64 | 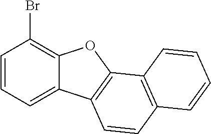 [SMILES: Brc1cccc2c1oc1c3ccccc3ccc21] | 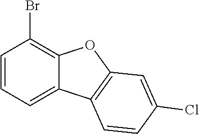 [SMILES: Clc1ccc2c(c1)oc1c(Br)cccc12] |
| 1-65 | 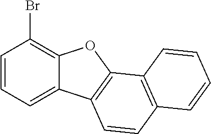 [SMILES: Brc1cccc2c1oc1c3ccccc3ccc21] | 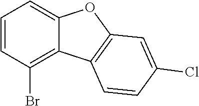 [SMILES: Clc1ccc2c(c1)oc1cccc(Br)c12] |
| 1-66 | 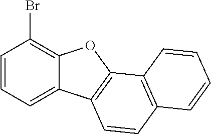 [SMILES: Brc1cccc2c1oc1c3ccccc3ccc21] | 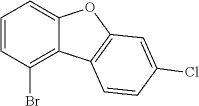 [SMILES: Clc1ccc2c(c1)oc1cccc(Br)c12] |
| 1-67 | 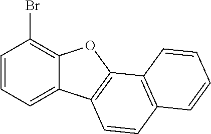 [SMILES: Brc1cccc2c1oc1c3ccccc3ccc21] | 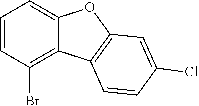 [SMILES: Clc1ccc2c(c1)oc1cccc(Br)c12] |
| 1-68 | 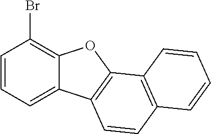 [SMILES: Brc1cccc2c1oc1c3ccccc3ccc21] | 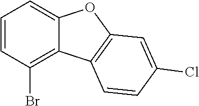 [SMILES: Clc1ccc2c(c1)oc1cccc(Br)c12] |
| 1-69 | 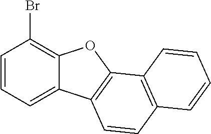 [SMILES: Brc1cccc2c1oc1c3ccccc3ccc21] | 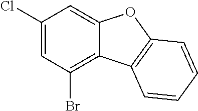 [SMILES: Clc1cc(Br)c2c(c1)oc1ccccc12] |
| 1-70 | 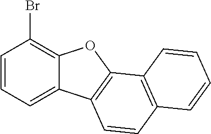 [SMILES: Brc1cccc2c1oc1c3ccccc3ccc21] | 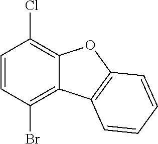 [SMILES: Clc1ccc(Br)c2c1oc1ccccc12] |
| 1-71 | 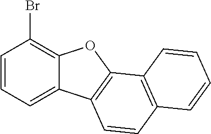 [SMILES: Brc1cccc2c1oc1c3ccccc3ccc21] | 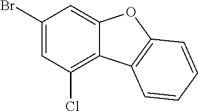 [SMILES: Clc1cc(Br)cc2oc3ccccc3c12] |
| 1-72 | 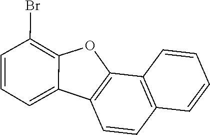 [SMILES: Brc1cccc2c1oc1c3ccccc3ccc21] | 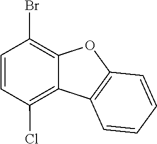 [SMILES: Clc1ccc(Br)c2oc3ccccc3c12] |
| 1-81 | 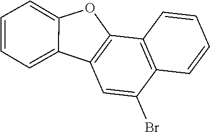 [SMILES: Brc1cc2c3ccccc3oc2c2ccccc12] | 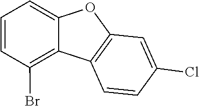 [SMILES: Clc1ccc2c(c1)oc1cccc(Br)c12] |
| 1-82 | 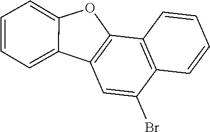 [SMILES: Brc1cc2c3ccccc3oc2c2ccccc12] | 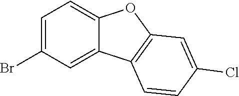 [SMILES: Clc1ccc2c(c1)oc1ccc(Br)cc12] |
| 1-83 | 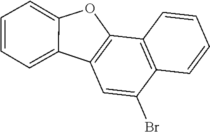 [SMILES: Brc1cc2c3ccccc3oc2c2ccccc12] | 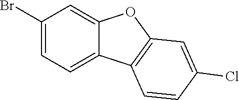 [SMILES: Clc1ccc2c(c1)oc1cc(Br)ccc12] |
| 1-84 | 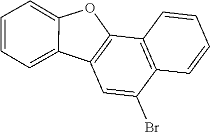 [SMILES: Brc1cc2c3ccccc3oc2c2ccccc12] | 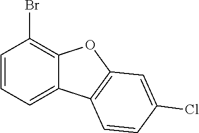 [SMILES: Clc1ccc2c(c1)oc1c(Br)cccc12] |
| 1-85 | 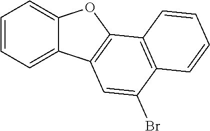 [SMILES: Brc1cc2c3ccccc3oc2c2ccccc12] | 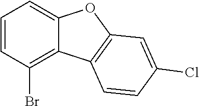 [SMILES: Clc1ccc2c(c1)oc1cccc(Br)c12] |
| 1-86 | 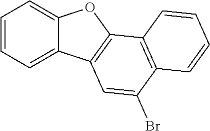 [SMILES: Brc1cc2c3ccccc3oc2c2ccccc12] | 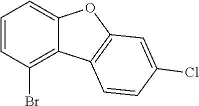 [SMILES: Clc1ccc2c(c1)oc1cccc(Br)c12] |
| 1-87 | 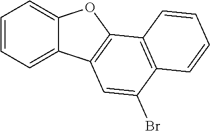 [SMILES: Brc1cc2c3ccccc3oc2c2ccccc12] | 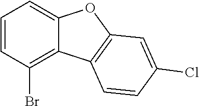 [SMILES: Clc1ccc2c(c1)oc1cccc(Br)c12] |
| 1-88 | 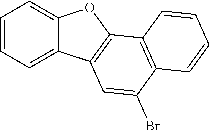 [SMILES: Brc1cc2c3ccccc3oc2c2ccccc12] | 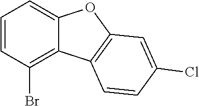 [SMILES: Clc1ccc2c(c1)oc1cccc(Br)c12] |
| 1-89 | 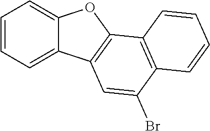 [SMILES: Brc1cc2c3ccccc3oc2c2ccccc12] | 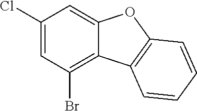 [SMILES: Clc1cc(Br)c2c(c1)oc1ccccc12] |
| 1-90 | 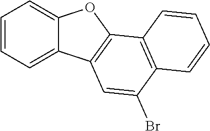 [SMILES: Brc1cc2c3ccccc3oc2c2ccccc12] | 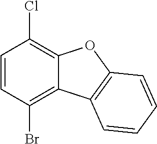 [SMILES: Clc1ccc(Br)c2c1oc1ccccc12] |
| 1-91 | 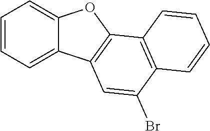 [SMILES: Brc1cc2c3ccccc3oc2c2ccccc12] | 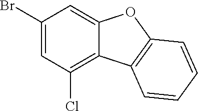 [SMILES: Clc1cc(Br)cc2oc3ccccc3c12] |
| 1-92 | 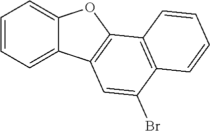 [SMILES: Brc1cc2c3ccccc3oc2c2ccccc12] | 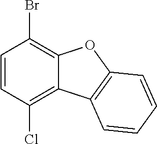 [SMILES: Clc1ccc(Br)c2oc3ccccc3c12] |
| 1-101 | 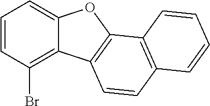 [SMILES: Brc1cccc2oc3c4ccccc4ccc3c12] | 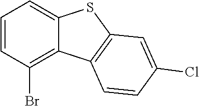 [SMILES: Clc1ccc2c(c1)sc1cccc(Br)c12] |
| 1-102 | 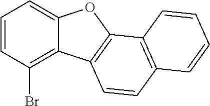 [SMILES: Brc1cccc2oc3c4ccccc4ccc3c12] | 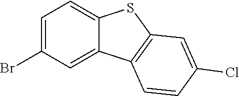 [SMILES: Clc1ccc2c(c1)sc1ccc(Br)cc12] |
| 1-103 | 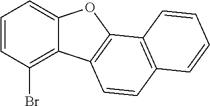 [SMILES: Brc1cccc2oc3c4ccccc4ccc3c12] | 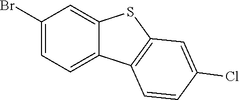 [SMILES: Clc1ccc2c(c1)sc1cc(Br)ccc12] |
| 1-104 | 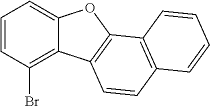 [SMILES: Brc1cccc2oc3c4ccccc4ccc3c12] | 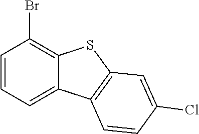 [SMILES: Clc1ccc2c(c1)sc1c(Br)cccc12] |
| 1-105 | 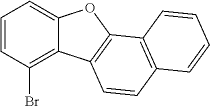 [SMILES: Brc1cccc2oc3c4ccccc4ccc3c12] | 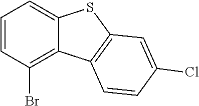 [SMILES: Clc1ccc2c(c1)sc1cccc(Br)c12] |
| 1-106 | 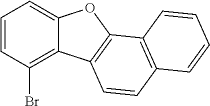 [SMILES: Brc1cccc2oc3c4ccccc4ccc3c12] | 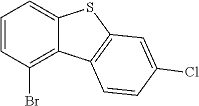 [SMILES: Clc1ccc2c(c1)sc1cccc(Br)c12] |
| 1-107 | 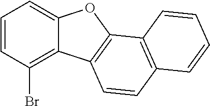 [SMILES: Brc1cccc2oc3c4ccccc4ccc3c12] | 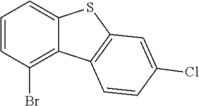 [SMILES: Clc1ccc2c(c1)sc1cccc(Br)c12] |
| 1-108 | 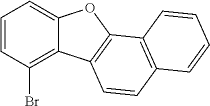 [SMILES: Brc1cccc2oc3c4ccccc4ccc3c12] | 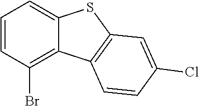 [SMILES: Clc1ccc2c(c1)sc1cccc(Br)c12] |
| 1-109 | 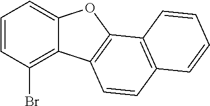 [SMILES: Brc1cccc2oc3c4ccccc4ccc3c12] | 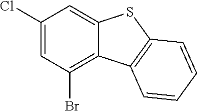 [SMILES: Clc1cc(Br)c2c(c1)sc1ccccc12] |
| 1-110 | 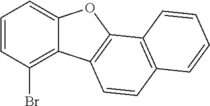 [SMILES: Brc1cccc2oc3c4ccccc4ccc3c12] | 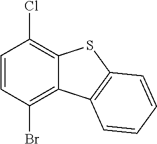 [SMILES: Clc1ccc(Br)c2c1sc1ccccc12] |
| 1-111 | 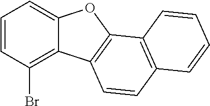 [SMILES: Brc1cccc2oc3c4ccccc4ccc3c12] | 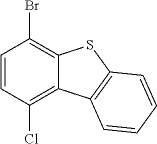 [SMILES: Clc1ccc(Br)c2sc3ccccc3c12] |
| 1-112 | 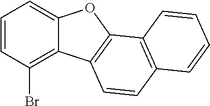 [SMILES: Brc1cccc2oc3c4ccccc4ccc3c12] | 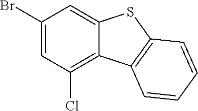 [SMILES: Clc1cc(Br)cc2sc3ccccc3c12] |
| 1-121 | 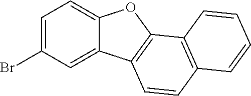 [SMILES: Brc1ccc2oc3c4ccccc4ccc3c2c1] | 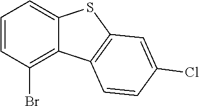 [SMILES: Clc1ccc2c(c1)sc1cccc(Br)c12] |
| 1-122 | 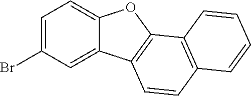 [SMILES: Brc1ccc2oc3c4ccccc4ccc3c2c1] | 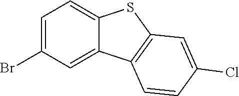 [SMILES: Clc1ccc2c(c1)sc1ccc(Br)cc12] |
| 1-123 | 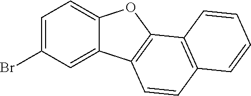 [SMILES: Brc1ccc2oc3c4ccccc4ccc3c2c1] | 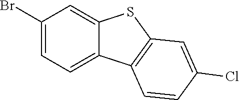 [SMILES: Clc1ccc2c(c1)sc1cc(Br)ccc12] |
| 1-124 | 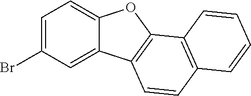 [SMILES: Brc1ccc2oc3c4ccccc4ccc3c2c1] | 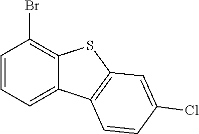 [SMILES: Clc1ccc2c(c1)sc1c(Br)cccc12] |
| 1-125 | 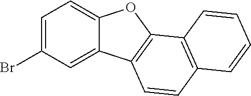 [SMILES: Brc1ccc2oc3c4ccccc4ccc3c2c1] | 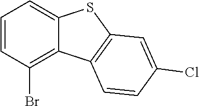 [SMILES: Clc1ccc2c(c1)sc1cccc(Br)c12] |
| 1-126 | 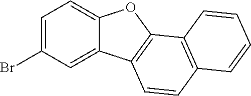 [SMILES: Brc1ccc2oc3c4ccccc4ccc3c2c1] | 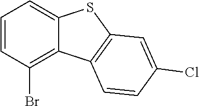 [SMILES: Clc1ccc2c(c1)sc1cccc(Br)c12] |
| 1-127 | 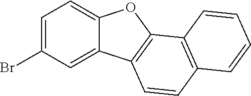 [SMILES: Brc1ccc2oc3c4ccccc4ccc3c2c1] | 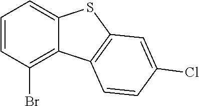 [SMILES: Clc1ccc2c(c1)sc1cccc(Br)c12] |
| 1-128 | 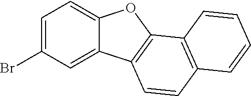 [SMILES: Brc1ccc2oc3c4ccccc4ccc3c2c1] | 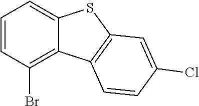 [SMILES: Clc1ccc2c(c1)sc1cccc(Br)c12] |
| 1-129 | 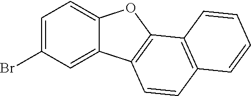 [SMILES: Brc1ccc2oc3c4ccccc4ccc3c2c1] | 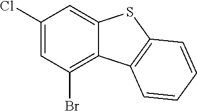 [SMILES: Clc1cc(Br)c2c(c1)sc1ccccc12] |
| 1-130 | 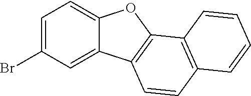 [SMILES: Brc1ccc2oc3c4ccccc4ccc3c2c1] | 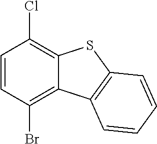 [SMILES: Clc1ccc(Br)c2c1sc1ccccc12] |
| 1-131 | 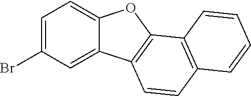 [SMILES: Brc1ccc2oc3c4ccccc4ccc3c2c1] | 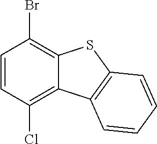 [SMILES: Clc1ccc(Br)c2sc3ccccc3c12] |
| 1-132 | 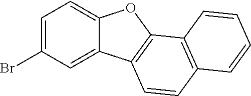 [SMILES: Brc1ccc2oc3c4ccccc4ccc3c2c1] | 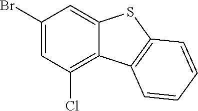 [SMILES: Clc1cc(Br)cc2sc3ccccc3c12] |
| 1-141 | 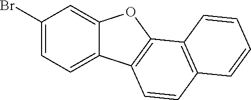 [SMILES: Brc1ccc2c(c1)oc1c3ccccc3ccc21] | 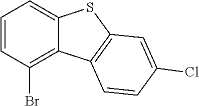 [SMILES: Clc1ccc2c(c1)sc1cccc(Br)c12] |
| 1-142 | 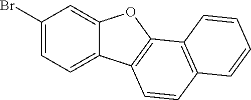 [SMILES: Brc1ccc2c(c1)oc1c3ccccc3ccc21] | 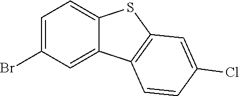 [SMILES: Clc1ccc2c(c1)sc1ccc(Br)cc12] |
| 1-143 | 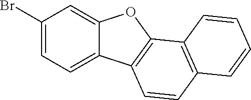 [SMILES: Brc1ccc2c(c1)oc1c3ccccc3ccc21] | 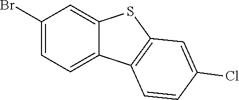 [SMILES: Clc1ccc2c(c1)sc1cc(Br)ccc12] |
| 1-144 | 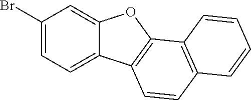 [SMILES: Brc1ccc2c(c1)oc1c3ccccc3ccc21] | 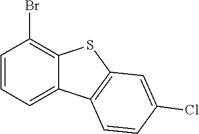 [SMILES: Clc1ccc2c(c1)sc1c(Br)cccc12] |
| 1-145 | 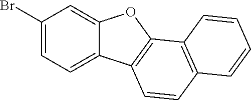 [SMILES: Brc1ccc2c(c1)oc1c3ccccc3ccc21] | 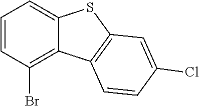 [SMILES: Clc1ccc2c(c1)sc1cccc(Br)c12] |
| 1-146 | 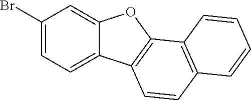 [SMILES: Brc1ccc2c(c1)oc1c3ccccc3ccc21] | 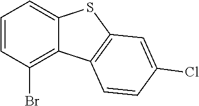 [SMILES: Clc1ccc2c(c1)sc1cccc(Br)c12] |
| 1-147 | 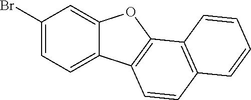 [SMILES: Brc1ccc2c(c1)oc1c3ccccc3ccc21] | 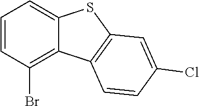 [SMILES: Clc1ccc2c(c1)sc1cccc(Br)c12] |
| 1-148 | 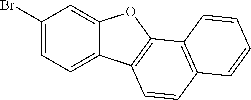 [SMILES: Brc1ccc2c(c1)oc1c3ccccc3ccc21] | 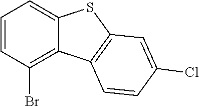 [SMILES: Clc1ccc2c(c1)sc1cccc(Br)c12] |
| 1-149 | 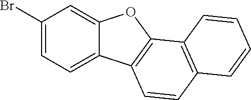 [SMILES: Brc1ccc2c(c1)oc1c3ccccc3ccc21] | 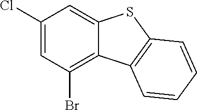 [SMILES: Clc1cc(Br)c2c(c1)sc1ccccc12] |
| 1-150 | 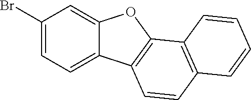 [SMILES: Brc1ccc2c(c1)oc1c3ccccc3ccc21] | 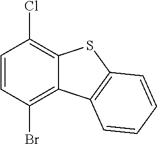 [SMILES: Clc1ccc(Br)c2c1sc1ccccc12] |
| 1-151 | 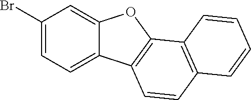 [SMILES: Brc1ccc2c(c1)oc1c3ccccc3ccc21] | 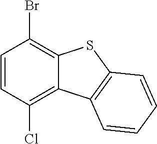 [SMILES: Clc1ccc(Br)c2sc3ccccc3c12] |
| 1-152 | 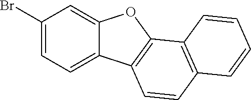 [SMILES: Brc1ccc2c(c1)oc1c3ccccc3ccc21] | 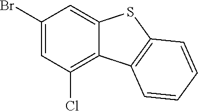 [SMILES: Clc1cc(Br)cc2sc3ccccc3c12] |
| 1-161 | 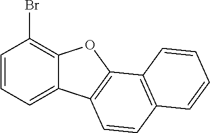 [SMILES: Brc1cccc2c1oc1c3ccccc3ccc21] | 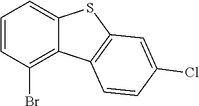 [SMILES: Clc1ccc2c(c1)sc1cccc(Br)c12] |
| 1-162 | 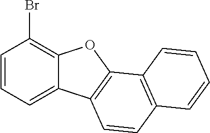 [SMILES: Brc1cccc2c1oc1c3ccccc3ccc21] | 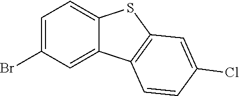 [SMILES: Clc1ccc2c(c1)sc1ccc(Br)cc12] |
| 1-163 | 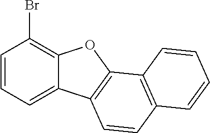 [SMILES: Brc1cccc2c1oc1c3ccccc3ccc21] | 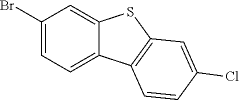 [SMILES: Clc1ccc2c(c1)sc1cc(Br)ccc12] |
| 1-164 | 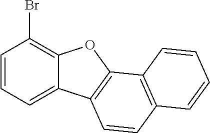 [SMILES: Brc1cccc2c1oc1c3ccccc3ccc21] | 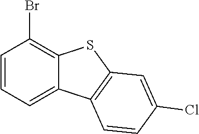 [SMILES: Clc1ccc2c(c1)sc1c(Br)cccc12] |
| 1-165 | 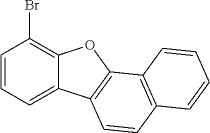 [SMILES: Brc1cccc2c1oc1c3ccccc3ccc21] | 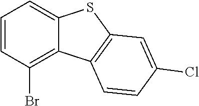 [SMILES: Clc1ccc2c(c1)sc1cccc(Br)c12] |
| 1-166 | 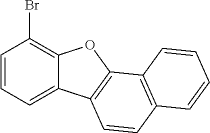 [SMILES: Brc1cccc2c1oc1c3ccccc3ccc21] | 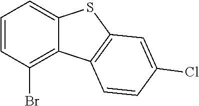 [SMILES: Clc1ccc2c(c1)sc1cccc(Br)c12] |
| 1-167 | 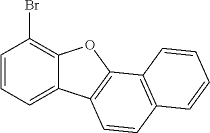 [SMILES: Brc1cccc2c1oc1c3ccccc3ccc21] | 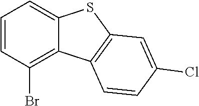 [SMILES: Clc1ccc2c(c1)sc1cccc(Br)c12] |
| 1-168 | 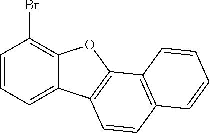 [SMILES: Brc1cccc2c1oc1c3ccccc3ccc21] | 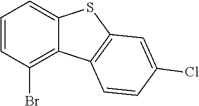 [SMILES: Clc1ccc2c(c1)sc1cccc(Br)c12] |
| 1-169 | 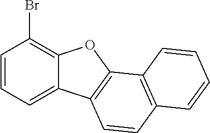 [SMILES: Brc1cccc2c1oc1c3ccccc3ccc21] | 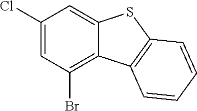 [SMILES: Clc1cc(Br)c2c(c1)sc1ccccc12] |
| 1-170 | 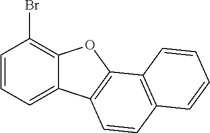 [SMILES: Brc1cccc2c1oc1c3ccccc3ccc21] | 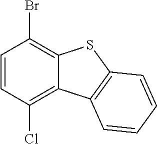 [SMILES: Clc1ccc(Br)c2sc3ccccc3c12] |
| 1-171 | 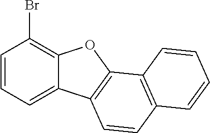 [SMILES: Brc1cccc2c1oc1c3ccccc3ccc21] | 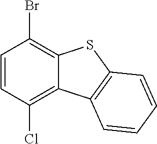 [SMILES: Clc1ccc(Br)c2sc3ccccc3c12] |
| 1-172 | 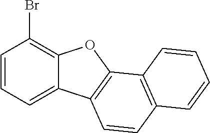 [SMILES: Brc1cccc2c1oc1c3ccccc3ccc21] | 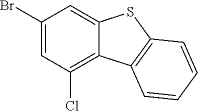 [SMILES: Clc1cc(Br)cc2sc3ccccc3c12] |
| 1-181 | 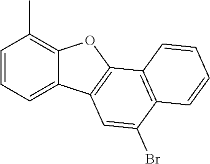 [SMILES: Cc1cccc2c1oc1c3ccccc3c(Br)cc21] | 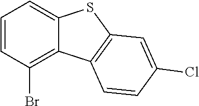 [SMILES: Clc1ccc2c(c1)sc1cccc(Br)c12] |
| 1-182 | 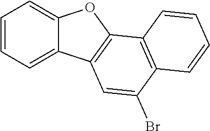 [SMILES: Brc1cc2c3ccccc3oc2c2ccccc12] | 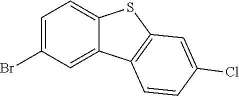 [SMILES: Clc1ccc2c(c1)sc1ccc(Br)cc12] |
| 1-183 | 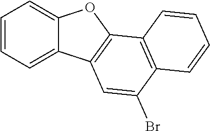 [SMILES: Brc1cc2c3ccccc3oc2c2ccccc12] | 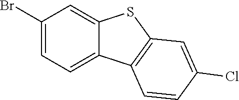 [SMILES: Clc1ccc2c(c1)sc1cc(Br)ccc12] |
| 1-184 | 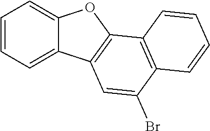 [SMILES: Brc1cc2c3ccccc3oc2c2ccccc12] | 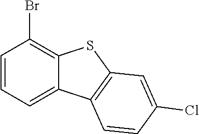 [SMILES: Clc1ccc2c(c1)sc1c(Br)cccc12] |
| 1-185 | 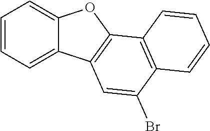 [SMILES: Brc1cc2c3ccccc3oc2c2ccccc12] | 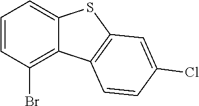 [SMILES: Clc1ccc2c(c1)sc1cccc(Br)c12] |
| 1-186 | 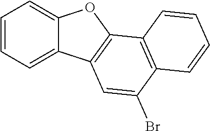 [SMILES: Brc1cc2c3ccccc3oc2c2ccccc12] | 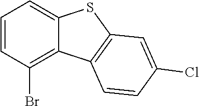 [SMILES: Clc1ccc2c(c1)sc1cccc(Br)c12] |
| 1-187 | 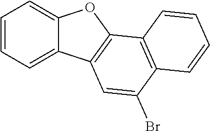 [SMILES: Brc1cc2c3ccccc3oc2c2ccccc12] | 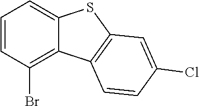 [SMILES: Clc1ccc2c(c1)sc1cccc(Br)c12] |
| 1-188 | 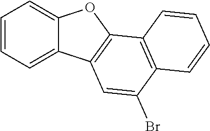 [SMILES: Brc1cc2c3ccccc3oc2c2ccccc12] | 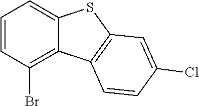 [SMILES: Clc1ccc2c(c1)sc1cccc(Br)c12] |
| 1-189 | 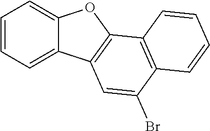 [SMILES: Brc1cc2c3ccccc3oc2c2ccccc12] | 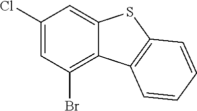 [SMILES: Clc1cc(Br)c2c(c1)sc1ccccc12] |
| 1-190 | 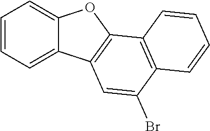 [SMILES: Brc1cc2c3ccccc3oc2c2ccccc12] | 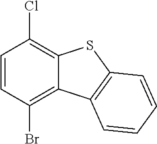 [SMILES: Clc1ccc(Br)c2c1sc1ccccc12] |
| 1-191 | 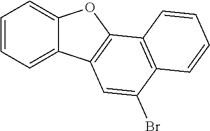 [SMILES: Brc1cc2c3ccccc3oc2c2ccccc12] | 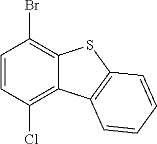 [SMILES: Clc1ccc(Br)c2sc3ccccc3c12] |
| 1-192 | 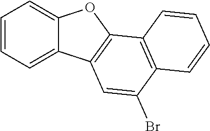 [SMILES: Brc1cc2c3ccccc3oc2c2ccccc12] | 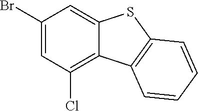 [SMILES: Clc1cc(Br)cc2sc3ccccc3c12] |
| 1-201 | 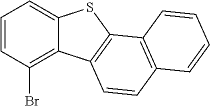 [SMILES: Brc1cccc2sc3c4ccccc4ccc3c12] | 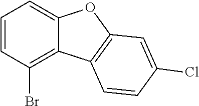 [SMILES: Clc1ccc2c(c1)oc1cccc(Br)c12] |
| 1-202 | 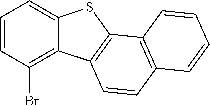 [SMILES: Brc1cccc2sc3c4ccccc4ccc3c12] | 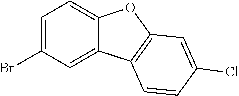 [SMILES: Clc1ccc2c(c1)oc1ccc(Br)cc12] |
| 1-203 | 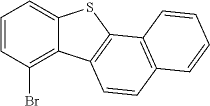 [SMILES: Brc1cccc2sc3c4ccccc4ccc3c12] | 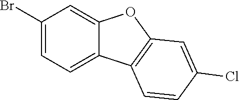 [SMILES: Clc1ccc2c(c1)oc1cc(Br)ccc12] |
| 1-204 | 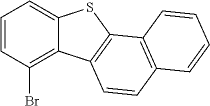 [SMILES: Brc1cccc2sc3c4ccccc4ccc3c12] | 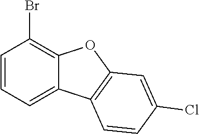 [SMILES: Clc1ccc2c(c1)oc1c(Br)cccc12] |
| 1-205 | 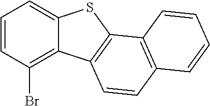 [SMILES: Brc1cccc2sc3c4ccccc4ccc3c12] | 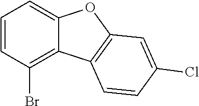 [SMILES: Clc1ccc2c(c1)oc1cccc(Br)c12] |
| 1-206 | 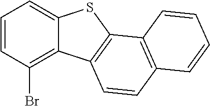 [SMILES: Brc1cccc2sc3c4ccccc4ccc3c12] | 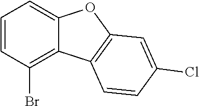 [SMILES: Clc1ccc2c(c1)oc1cccc(Br)c12] |
| 1-207 | 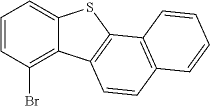 [SMILES: Brc1cccc2sc3c4ccccc4ccc3c12] | 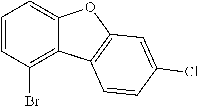 [SMILES: Clc1ccc2c(c1)oc1cccc(Br)c12] |
| 1-208 | 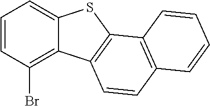 [SMILES: Brc1cccc2sc3c4ccccc4ccc3c12] | 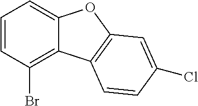 [SMILES: Clc1ccc2c(c1)oc1cccc(Br)c12] |
| 1-213 | 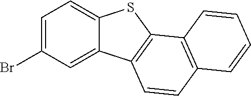 [SMILES: Brc1ccc2sc3c4ccccc4ccc3c2c1] | 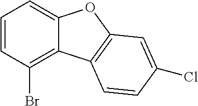 [SMILES: Clc1ccc2c(c1)oc1cccc(Br)c12] |
| 1-214 | 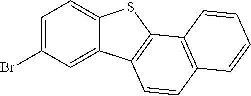 [SMILES: Brc1ccc2sc3c4ccccc4ccc3c2c1] | 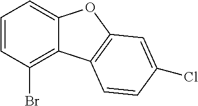 [SMILES: Clc1ccc2c(c1)oc1cccc(Br)c12] |
| 1-215 | 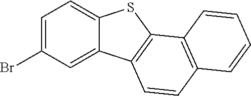 [SMILES: Brc1ccc2sc3c4ccccc4ccc3c2c1] | 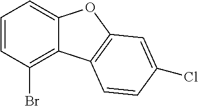 [SMILES: Clc1ccc2c(c1)oc1cccc(Br)c12] |
| 1-216 | 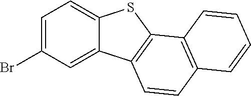 [SMILES: Brc1ccc2sc3c4ccccc4ccc3c2c1] | 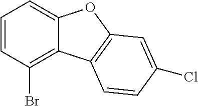 [SMILES: Clc1ccc2c(c1)oc1cccc(Br)c12] |
| 1-217 | 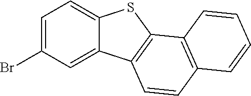 [SMILES: Brc1ccc2sc3c4ccccc4ccc3c2c1] | 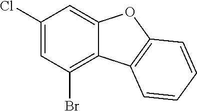 [SMILES: Clc1cc(Br)c2c(c1)oc1ccccc12] |
| 1-218 | 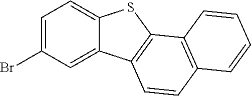 [SMILES: Brc1ccc2sc3c4ccccc4ccc3c2c1] | 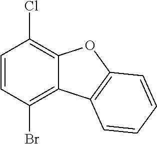 [SMILES: Clc1ccc(Br)c2c1oc1ccccc12] |
| 1-221 | 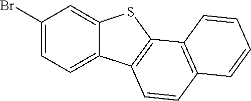 [SMILES: Brc1ccc2c(c1)sc1c3ccccc3ccc21] | 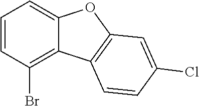 [SMILES: Clc1ccc2c(c1)oc1cccc(Br)c12] |
| 1-222 | 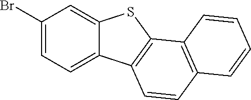 [SMILES: Brc1ccc2c(c1)sc1c3ccccc3ccc21] | 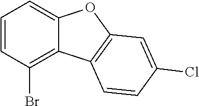 [SMILES: Clc1ccc2c(c1)oc1cccc(Br)c12] |
| 1-223 | 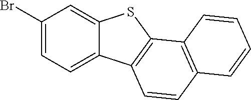 [SMILES: Brc1ccc2c(c1)sc1c3ccccc3ccc21] | 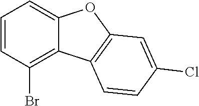 [SMILES: Clc1ccc2c(c1)oc1cccc(Br)c12] |
| 1-224 | 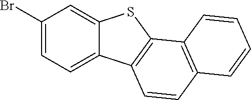 [SMILES: Brc1ccc2c(c1)sc1c3ccccc3ccc21] | 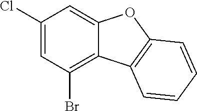 [SMILES: Clc1cc(Br)c2c(c1)oc1ccccc12] |
| 1-229 | 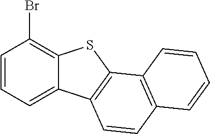 [SMILES: Brc1cccc2c1sc1c3ccccc3ccc21] | 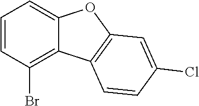 [SMILES: Clc1ccc2c(c1)oc1cccc(Br)c12] |
| 1-230 | 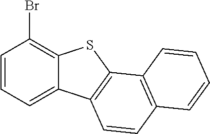 [SMILES: Brc1cccc2c1sc1c3ccccc3ccc21] | 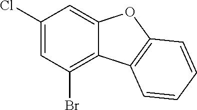 [SMILES: Clc1cc(Br)c2c(c1)oc1ccccc12] |
| 1-231 | 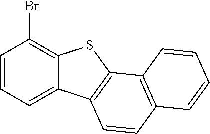 [SMILES: Brc1cccc2c1sc1c3ccccc3ccc21] | 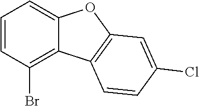 [SMILES: Clc1ccc2c(c1)oc1cccc(Br)c12] |
| 1-232 | 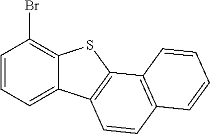 [SMILES: Brc1cccc2c1sc1c3ccccc3ccc21] | 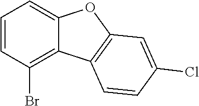 [SMILES: Clc1ccc2c(c1)oc1cccc(Br)c12] |
| 1-237 | 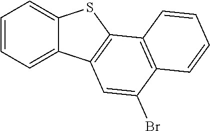 [SMILES: Brc1cc2c3ccccc3sc2c2ccccc12] | 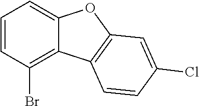 [SMILES: Clc1ccc2c(c1)oc1cccc(Br)c12] |
| 1-238 | 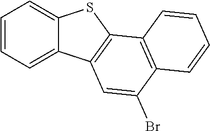 [SMILES: Brc1cc2c3ccccc3sc2c2ccccc12] | 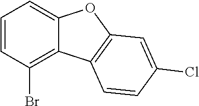 [SMILES: Clc1ccc2c(c1)oc1cccc(Br)c12] |
| 1-242 | 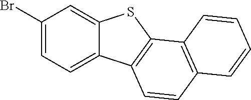 [SMILES: Brc1ccc2c(c1)sc1c3ccccc3ccc21] | 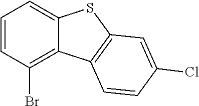 [SMILES: Clc1ccc2c(c1)sc1cccc(Br)c12] |
| 1-243 | 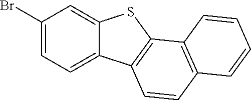 [SMILES: Brc1ccc2c(c1)sc1c3ccccc3ccc21] | 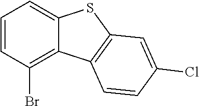 [SMILES: Clc1ccc2c(c1)sc1cccc(Br)c12] |
| 1-244 | 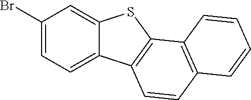 [SMILES: Brc1ccc2c(c1)sc1c3ccccc3ccc21] | 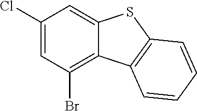 [SMILES: Clc1cc(Br)c2c(c1)sc1ccccc12] |
| 1-249 | 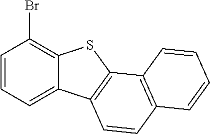 [SMILES: Brc1cccc2c1sc1c3ccccc3ccc21] | 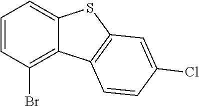 [SMILES: Clc1ccc2c(c1)sc1cccc(Br)c12] |
| 1-250 | 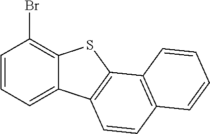 [SMILES: Brc1cccc2c1sc1c3ccccc3ccc21] | 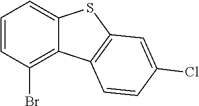 [SMILES: Clc1ccc2c(c1)sc1cccc(Br)c12] |
| 1-251 | 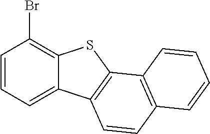 [SMILES: Brc1cccc2c1sc1c3ccccc3ccc21] | 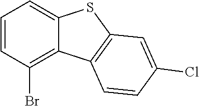 [SMILES: Clc1ccc2c(c1)sc1cccc(Br)c12] |
| 1-252 | 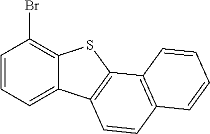 [SMILES: Brc1cccc2c1sc1c3ccccc3ccc21] | 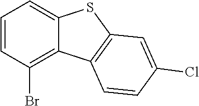 [SMILES: Clc1ccc2c(c1)sc1cccc(Br)c12] |
| 1-257 | 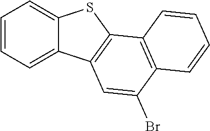 [SMILES: Brc1cc2c3ccccc3sc2c2ccccc12] | 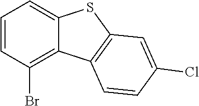 [SMILES: Clc1ccc2c(c1)sc1cccc(Br)c12] |
| 1-258 | 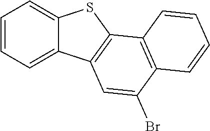 [SMILES: Brc1cc2c3ccccc3sc2c2ccccc12] | 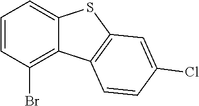 [SMILES: Clc1ccc2c(c1)sc1cccc(Br)c12] |

| Com- | | |
|---|---|---|
| pound | B-1 | compound C |
| 1-2 | 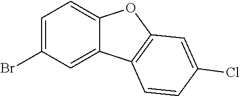 [SMILES: Clc1ccc2c(c1)oc1ccc(Br)cc12] | 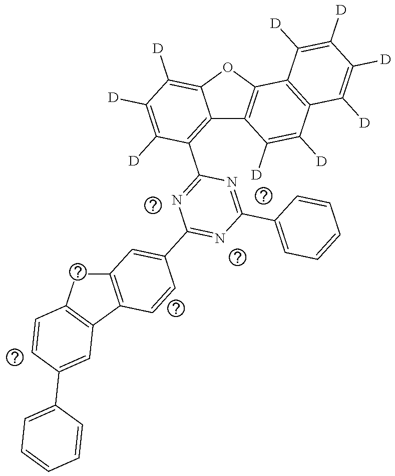 [SMILES: [2H]c1c([2H])c(-c2nc(-c3ccccc3)nc(-c3ccc4c(c3)Cc3ccc(-c5ccccc5)cc3-4)n2)c2c(oc3c4c([2H])c([2H])c([2H])c([2H])c4c([2H])c([2H])c32)c1[2H]] |
| 1-3 | 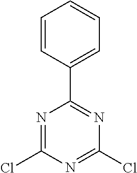 [SMILES: Clc1nc(Cl)nc(-c2ccccc2)n1] | 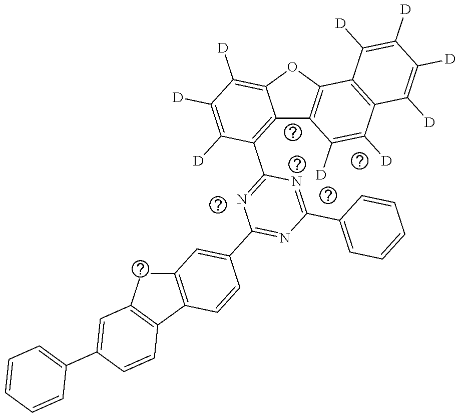 [SMILES: [2H]c1c([2H])c(-c2nc(-c3ccccc3)nc(-c3ccc4c(c3)Cc3cc(-c5ccccc5)ccc3-4)n2)c2c(oc3c4c([2H])c([2H])c([2H])c([2H])c4c([2H])c([2H])c32)c1[2H]] |
| 1-4 | 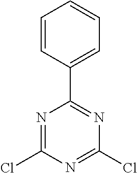 [SMILES: Clc1nc(Cl)nc(-c2ccccc2)n1] | 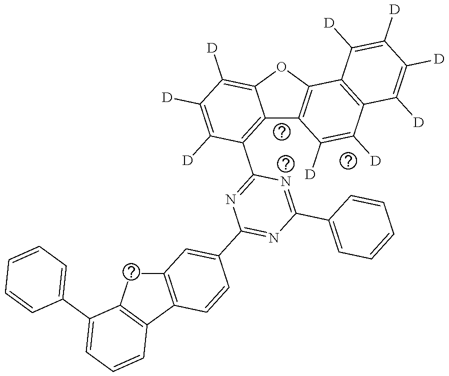 [SMILES: [2H]c1c([2H])c(-c2nc(-c3ccccc3)nc(-c3ccc4c(c3)Cc3c(-c5ccccc5)cccc3-4)n2)c2c(oc3c4c([2H])c([2H])c([2H])c([2H])c4c([2H])c([2H])c32)c1[2H]] |
| 1-5 | 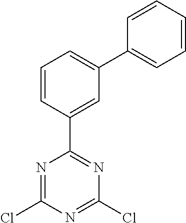 [SMILES: Clc1nc(Cl)nc(-c2cccc(-c3ccccc3)c2)n1] | 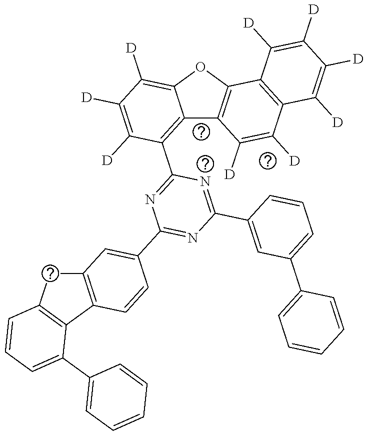 [SMILES: [2H]c1c([2H])c(-c2nc(-c3cccc(-c4ccccc4)c3)nc(-c3ccc4c(c3)Cc3cccc(-c5ccccc5)c3-4)n2)c2c(oc3c4c([2H])c([2H])c([2H])c([2H])c4c([2H])c([2H])c32)c1[2H]] |
| 1-6 | 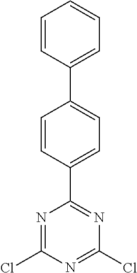 [SMILES: Clc1nc(Cl)nc(-c2ccc(-c3ccccc3)cc2)n1] | 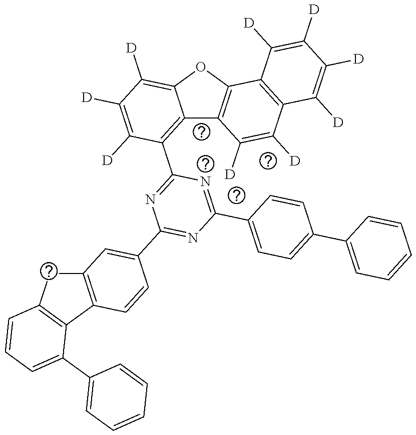 [SMILES: [2H]c1c([2H])c(-c2nc(-c3ccc(-c4ccccc4)cc3)nc(-c3ccc4c(c3)Cc3cccc(-c5ccccc5)c3-4)n2)c2c(oc3c4c([2H])c([2H])c([2H])c([2H])c4c([2H])c([2H])c32)c1[2H]] |
| 1-7 | 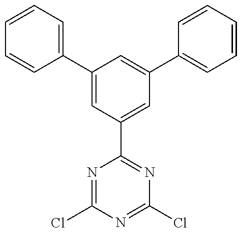 [SMILES: Clc1nc(Cl)nc(-c2cc(-c3ccccc3)cc(-c3ccccc3)c2)n1] | 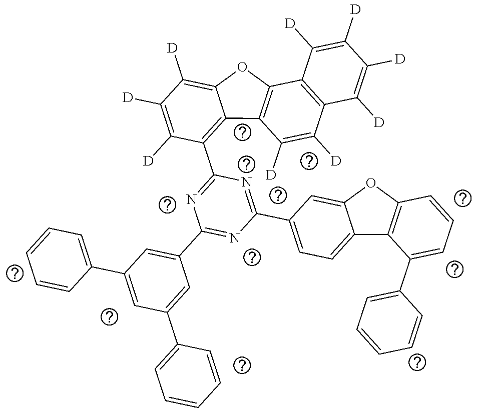 [SMILES: [2H]c1c([2H])c(-c2nc(-c3cc(-c4ccccc4)cc(-c4ccccc4)c3)nc(-c3ccc4c(c3)oc3cccc(-c5ccccc5)c34)n2)c2c(oc3c4c([2H])c([2H])c([2H])c([2H])c4c([2H])c([2H])c32)c1[2H]] |
| 1-8 | 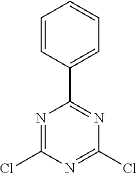 [SMILES: Clc1nc(Cl)nc(-c2ccccc2)n1] | 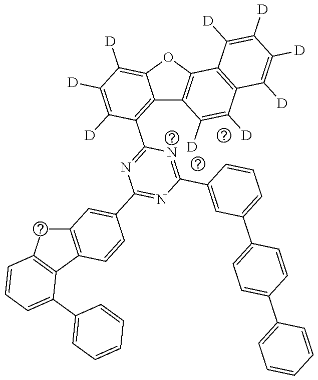 [SMILES: [2H]c1c([2H])c(-c2nc(-c3cccc(-c4ccc(-c5ccccc5)cc4)c3)nc(-c3ccc4c(c3)Cc3cccc(-c5ccccc5)c3-4)n2)c2c(oc3c4c([2H])c([2H])c([2H])c([2H])c4c([2H])c([2H])c32)c1[2H]] |
| 1-9 | 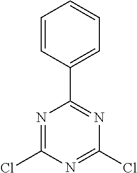 [SMILES: Clc1nc(Cl)nc(-c2ccccc2)n1] | 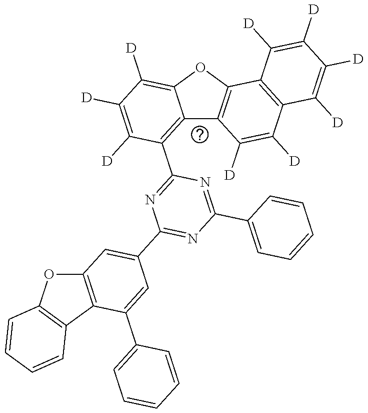 [SMILES: [2H]c1c([2H])c(-c2nc(-c3ccccc3)nc(-c3cc(-c4ccccc4)c4c(c3)oc3ccccc34)n2)c2c(oc3c4c([2H])c([2H])c([2H])c([2H])c4c([2H])c([2H])c32)c1[2H]] |
| 1-10 | 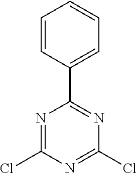 [SMILES: Clc1nc(Cl)nc(-c2ccccc2)n1] | 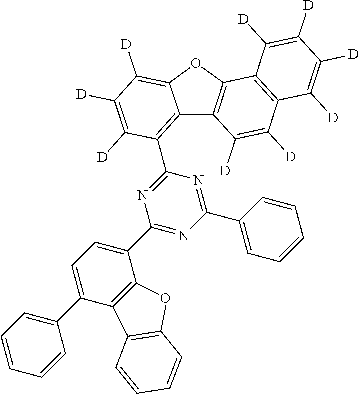 [SMILES: [2H]c1c([2H])c(-c2nc(-c3ccccc3)nc(-c3ccc(-c4ccccc4)c4c3oc3ccccc34)n2)c2c(oc3c4c([2H])c([2H])c([2H])c([2H])c4c([2H])c([2H])c32)c1[2H]] |
| 1-11 | 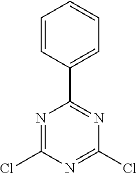 [SMILES: Clc1nc(Cl)nc(-c2ccccc2)n1] | 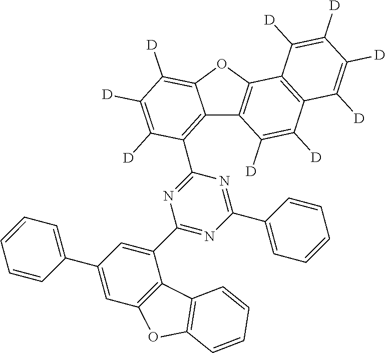 [SMILES: [2H]c1c([2H])c(-c2nc(-c3ccccc3)nc(-c3cc(-c4ccccc4)cc4oc5ccccc5c34)n2)c2c(oc3c4c([2H])c([2H])c([2H])c([2H])c4c([2H])c([2H])c32)c1[2H]] |
| 1-12 | 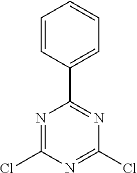 [SMILES: Clc1nc(Cl)nc(-c2ccccc2)n1] | 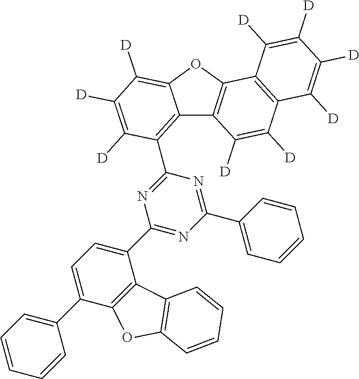 [SMILES: [2H]c1c([2H])c(-c2nc(-c3ccccc3)nc(-c3ccc(-c4ccccc4)c4oc5ccccc5c34)n2)c2c(oc3c4c([2H])c([2H])c([2H])c([2H])c4c([2H])c([2H])c32)c1[2H]] |
| 1-21 | 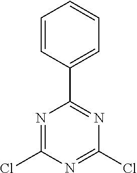 [SMILES: Clc1nc(Cl)nc(-c2ccccc2)n1] | 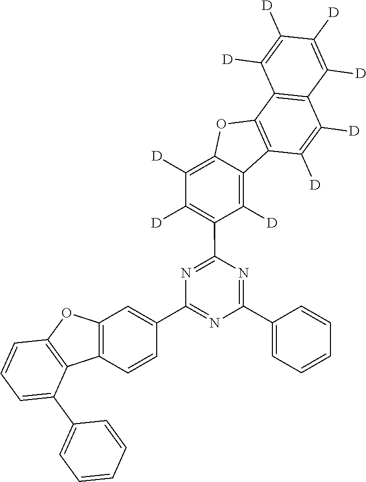 [SMILES: [2H]c1c(-c2nc(-c3ccccc3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]] |
| 1-22 | 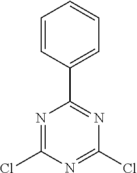 [SMILES: Clc1nc(Cl)nc(-c2ccccc2)n1] | 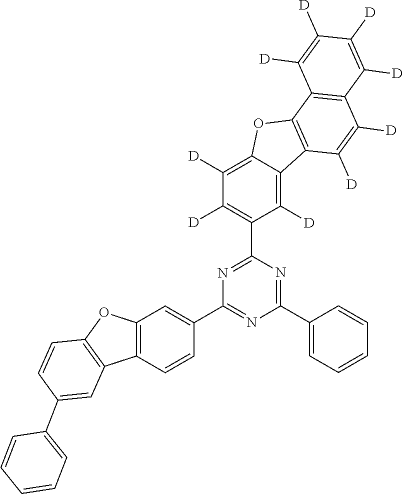 [SMILES: [2H]c1c(-c2nc(-c3ccccc3)nc(-c3ccc4c(c3)oc3ccc(-c5ccccc5)cc34)n2)c([2H])c2c(oc3c4c([2H])c([2H])c([2H])c([2H])c4c([2H])c([2H])c32)c1[2H]] |
| 1-23 | 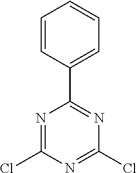 [SMILES: Clc1nc(Cl)nc(-c2ccccc2)n1] | 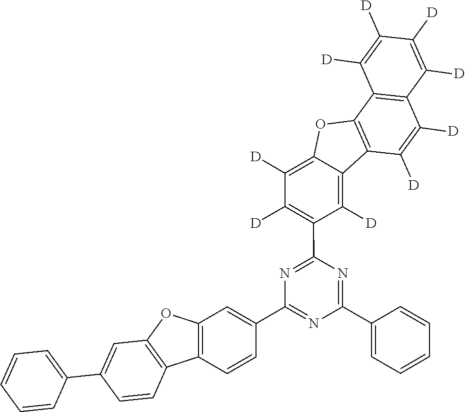 [SMILES: [2H]c1c(-c2nc(-c3ccccc3)nc(-c3ccc4c(c3)oc3cc(-c5ccccc5)ccc34)n2)c([2H])c2c(oc3c4c([2H])c([2H])c([2H])c([2H])c4c([2H])c([2H])c32)c1[2H]] |
| 1-24 | 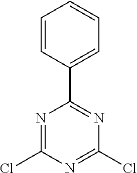 [SMILES: Clc1nc(Cl)nc(-c2ccccc2)n1] | 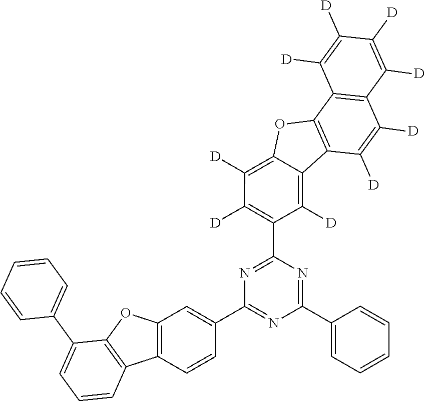 [SMILES: [2H]c1c(-c2nc(-c3ccccc3)nc(-c3ccc4c(c3)oc3c(-c5ccccc5)cccc34)n2)c([2H])c2c(oc3c4c([2H])c([2H])c([2H])c([2H])c4c([2H])c([2H])c32)c1[2H]] |
| 1-25 | 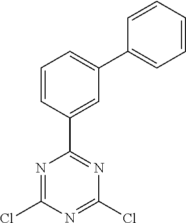 [SMILES: Clc1nc(Cl)nc(-c2cccc(-c3ccccc3)c2)n1] | 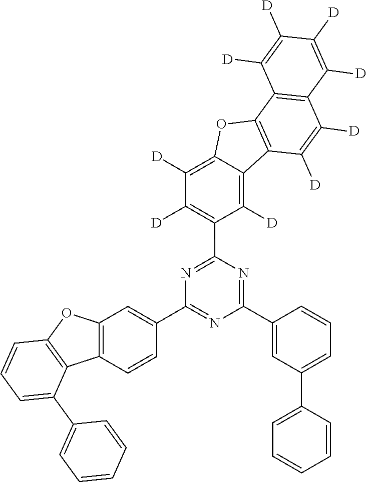 [SMILES: [2H]c1c(-c2nc(-c3cccc(-c4ccccc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]] |
| 1-26 | 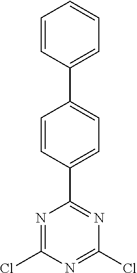 [SMILES: Clc1nc(Cl)nc(-c2ccc(-c3ccccc3)cc2)n1] | 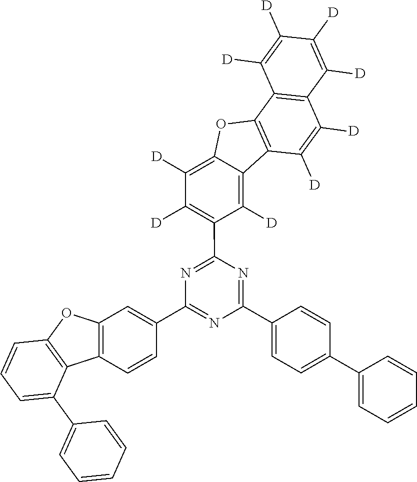 [SMILES: [2H]c1c(-c2nc(-c3ccc(-c4ccccc4)cc3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]] |
| 1-27 | 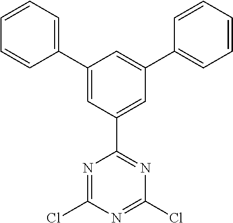 [SMILES: Clc1nc(Cl)nc(-c2cc(-c3ccccc3)cc(-c3ccccc3)c2)n1] | 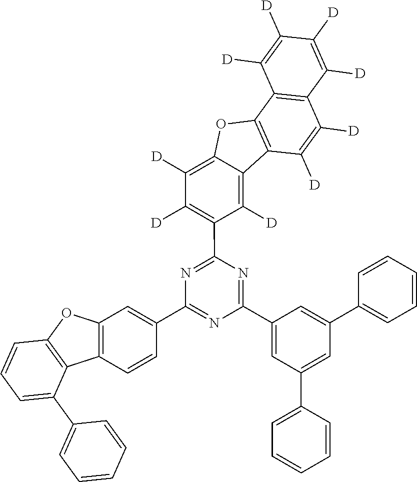 [SMILES: [2H]c1c(-c2nc(-c3cc(-c4ccccc4)cc(-c4ccccc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]] |
| 1-28 | 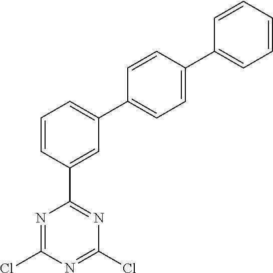 [SMILES: Clc1nc(Cl)nc(-c2cccc(-c3ccc(-c4ccccc4)cc3)c2)n1] | 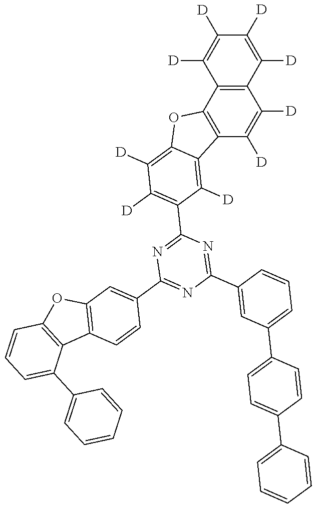 [SMILES: [2H]c1c(-c2nc(-c3cccc(-c4ccc(-c5ccccc5)cc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]] |
| 1-29 | 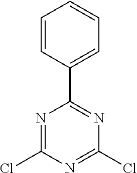 [SMILES: Clc1nc(Cl)nc(-c2ccccc2)n1] | 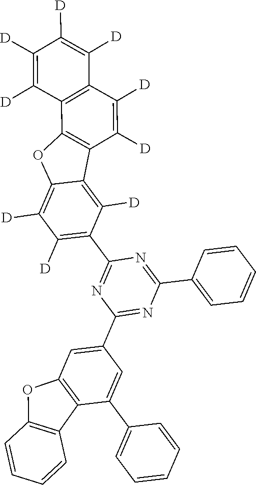 [SMILES: [2H]c1c(-c2nc(-c3ccccc3)nc(-c3cc(-c4ccccc4)c4c(c3)oc3ccccc34)n2)c([2H])c2c(oc3c4c([2H])c([2H])c([2H])c([2H])c4c([2H])c([2H])c32)c1[2H]] |
| 1-30 | 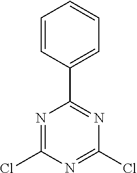 [SMILES: Clc1nc(Cl)nc(-c2ccccc2)n1] | 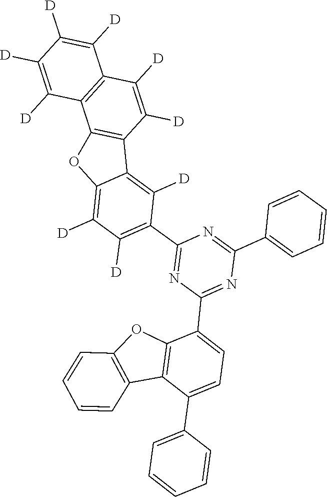 [SMILES: [2H]c1c(-c2nc(-c3ccccc3)nc(-c3ccc(-c4ccccc4)c4c3oc3ccccc34)n2)c([2H])c2c(oc3c4c([2H])c([2H])c([2H])c([2H])c4c([2H])c([2H])c32)c1[2H]] |
| 1-31 | 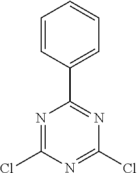 [SMILES: Clc1nc(Cl)nc(-c2ccccc2)n1] | 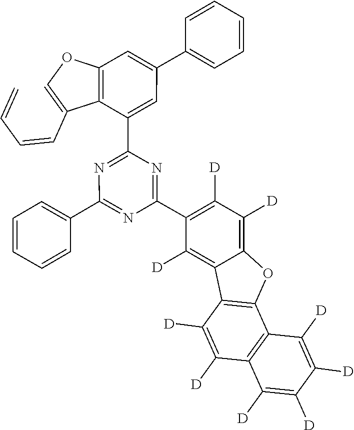 [SMILES: [2H]c1c(-c2nc(-c3ccccc3)nc(-c3cc(-c4ccccc4)cc4occ(/C=C\C=C)c34)n2)c([2H])c2c(oc3c4c([2H])c([2H])c([2H])c([2H])c4c([2H])c([2H])c32)c1[2H]] |
| 1-32 | 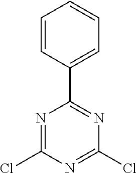 [SMILES: Clc1nc(Cl)nc(-c2ccccc2)n1] | 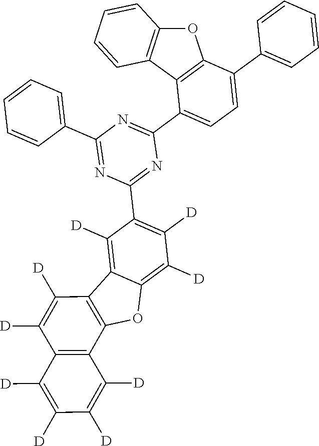 [SMILES: [2H]c1c(-c2nc(-c3ccccc3)nc(-c3ccc(-c4ccccc4)c4oc5ccccc5c34)n2)c([2H])c2c(oc3c4c([2H])c([2H])c([2H])c([2H])c4c([2H])c([2H])c32)c1[2H]] |
| 1-41 | 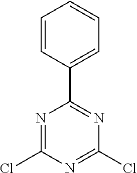 [SMILES: Clc1nc(Cl)nc(-c2ccccc2)n1] | 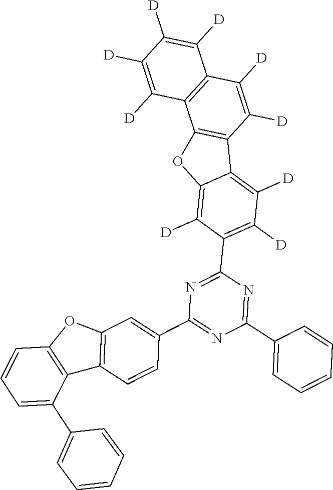 [SMILES: [2H]c1c([2H])c([2H])c2c(c1[2H])c([2H])c([2H])c1c2oc2c([2H])c(-c3nc(-c4ccccc4)nc(-c4ccc5c(c4)oc4cccc(-c6ccccc6)c45)n3)c([2H])c([2H])c21] |
| 1-42 | 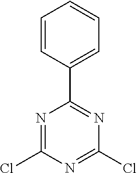 [SMILES: Clc1nc(Cl)nc(-c2ccccc2)n1] | 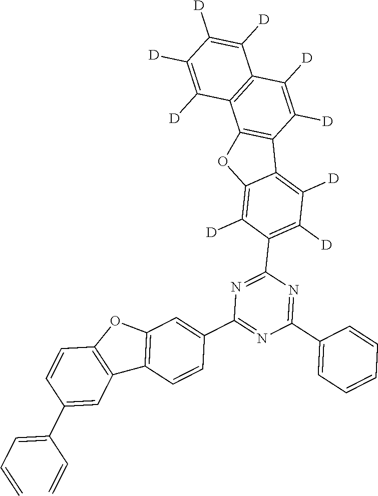 [SMILES: [2H]c1c([2H])c([2H])c2c(c1[2H])c([2H])c([2H])c1c2oc2c([2H])c(-c3nc(-c4ccccc4)nc(-c4ccc5c(c4)oc4ccc(/C(C=C)=C/C=C)cc45)n3)c([2H])c([2H])c21] |
| 1-43 | 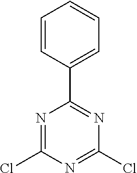 [SMILES: Clc1nc(Cl)nc(-c2ccccc2)n1] | 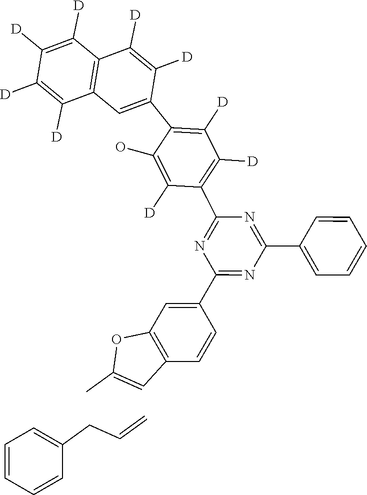 |
| 1-44 | 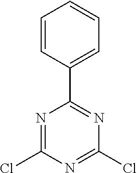 [SMILES: Clc1nc(Cl)nc(-c2ccccc2)n1] | 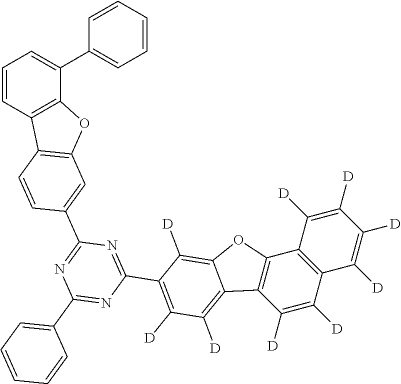 [SMILES: [2H]c1c([2H])c([2H])c2c(c1[2H])c([2H])c([2H])c1c2oc2c([2H])c(-c3nc(-c4ccccc4)nc(-c4ccc5c(c4)oc4c(-c6ccccc6)cccc45)n3)c([2H])c([2H])c21] |
| 1-45 | 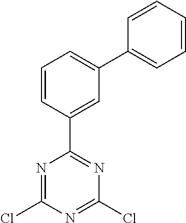 [SMILES: Clc1nc(Cl)nc(-c2cccc(-c3ccccc3)c2)n1] | 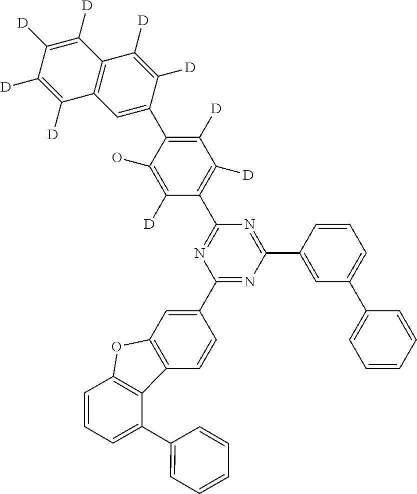 [SMILES: [2H]c1c([2H])c(-c2cc3c([2H])c([2H])c([2H])c([2H])c3c([2H])c2[2H])c(O)c([2H])c1-c1nc(-c2cccc(-c3ccccc3)c2)nc(-c2ccc3c(c2)oc2cccc(-c4ccccc4)c23)n1] |
| 1-46 | 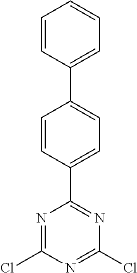 [SMILES: Clc1nc(Cl)nc(-c2ccc(-c3ccccc3)cc2)n1] | 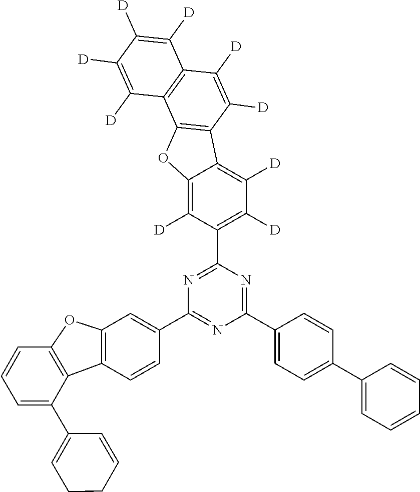 [SMILES: [2H]c1c([2H])c([2H])c2c(c1[2H])c([2H])c([2H])c1c2oc2c([2H])c(-c3nc(-c4ccc(-c5ccccc5)cc4)nc(-c4ccc5c(c4)oc4cccc(C6=CCCC=C6)c45)n3)c([2H])c([2H])c21] |
| 1-47 | 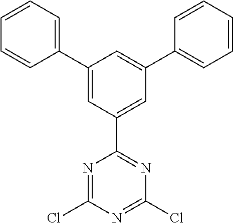 [SMILES: Clc1nc(Cl)nc(-c2cc(-c3ccccc3)cc(-c3ccccc3)c2)n1] | 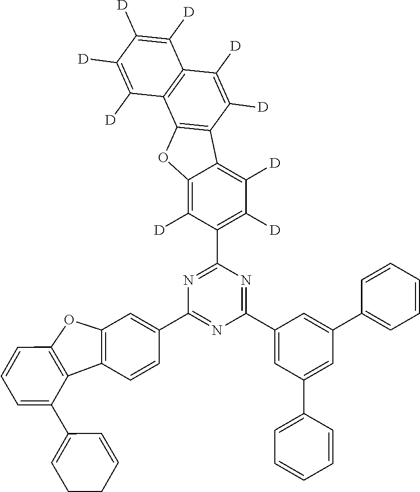 [SMILES: [2H]c1c([2H])c([2H])c2c(c1[2H])c([2H])c([2H])c1c2oc2c([2H])c(-c3nc(-c4cc(-c5ccccc5)cc(-c5ccccc5)c4)nc(-c4ccc5c(c4)oc4cccc(C6=CCCC=C6)c45)n3)c([2H])c([2H])c21] |
| 1-48 | 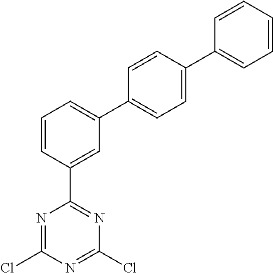 [SMILES: Clc1nc(Cl)nc(-c2cccc(-c3ccc(-c4ccccc4)cc3)c2)n1] | 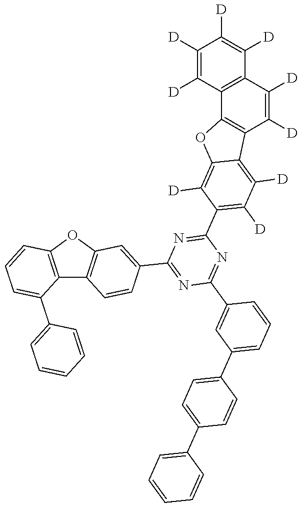 [SMILES: [2H]c1c([2H])c([2H])c2c(c1[2H])c([2H])c([2H])c1c2oc2c([2H])c(-c3nc(-c4cccc(-c5ccc(-c6ccccc6)cc5)c4)nc(-c4ccc5c(c4)oc4cccc(-c6ccccc6)c45)n3)c([2H])c([2H])c21] |
| 1-49 | 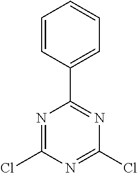 [SMILES: Clc1nc(Cl)nc(-c2ccccc2)n1] | 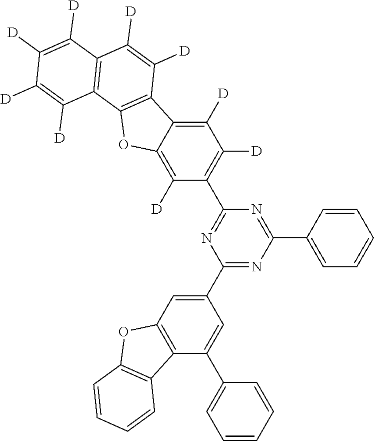 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)c5c(c4)oc4ccccc45)n3)c([2H])c([2H])c21] |
| 1-50 | 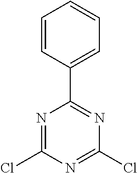 [SMILES: Clc1nc(Cl)nc(-c2ccccc2)n1] | 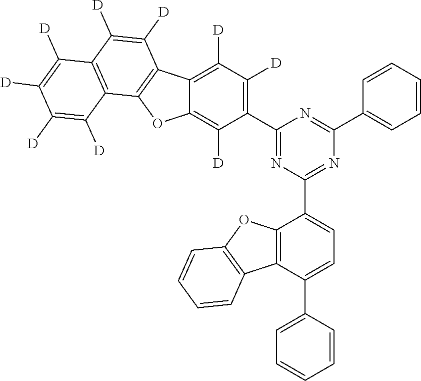 [SMILES: [2H]c1c([2H])c([2H])c2c(c1[2H])c([2H])c([2H])c1c2oc2c([2H])c(-c3nc(-c4ccccc4)nc(-c4ccc(-c5ccccc5)c5c4oc4ccccc45)n3)c([2H])c([2H])c21] |
| 1-51 | 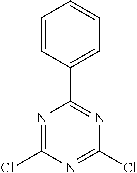 [SMILES: Clc1nc(Cl)nc(-c2ccccc2)n1] | 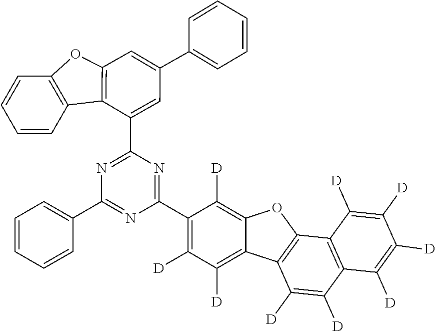 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)cc5oc6ccccc6c45)n3)c([2H])c([2H])c21] |
| 1-52 | 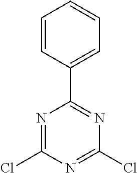 [SMILES: Clc1nc(Cl)nc(-c2ccccc2)n1] | 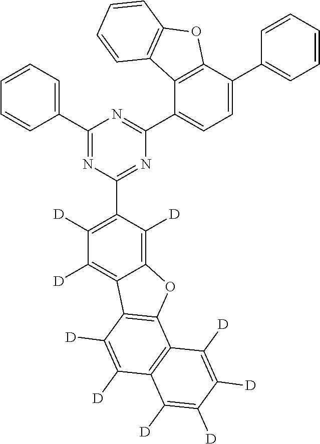 [SMILES: [2H]c1c([2H])c([2H])c2c(c1[2H])c([2H])c([2H])c1c2oc2c([2H])c(-c3nc(-c4ccccc4)nc(-c4ccc(-c5ccccc5)c5oc6ccccc6c45)n3)c([2H])c([2H])c21] |
| 1-61 | 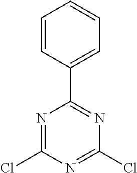 [SMILES: Clc1nc(Cl)nc(-c2ccccc2)n1] | 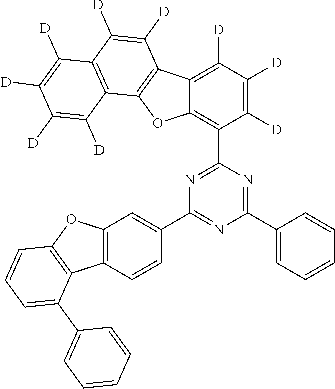 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2cccc(-c4ccccc4)c23)n1] |
| 1-62 | 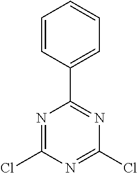 [SMILES: Clc1nc(Cl)nc(-c2ccccc2)n1] | 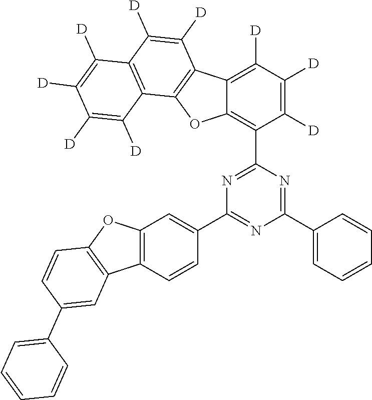 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2ccc(-c4ccccc4)cc23)n1] |
| 1-63 | 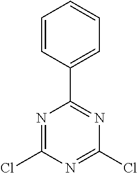 [SMILES: Clc1nc(Cl)nc(-c2ccccc2)n1] | 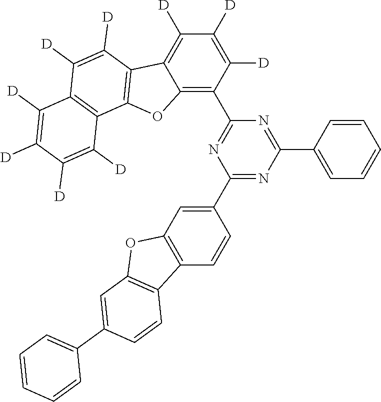 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2cc(-c4ccccc4)ccc23)n1] |
| 1-64 | 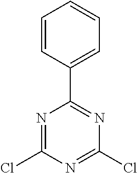 [SMILES: Clc1nc(Cl)nc(-c2ccccc2)n1] | 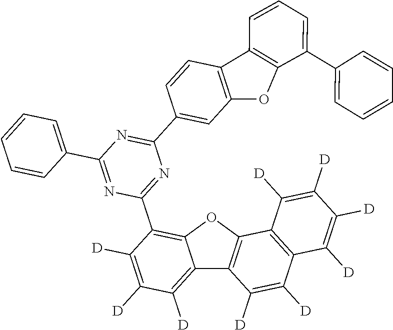 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2c(-c4ccccc4)cccc23)n1] |
| 1-65 | 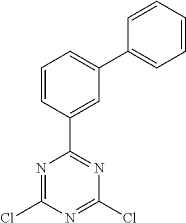 [SMILES: Clc1nc(Cl)nc(-c2cccc(-c3ccccc3)c2)n1] | 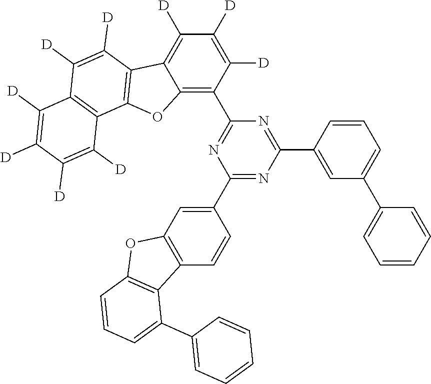 [SMILES: [2H]c1c([2H])c([2H])c2c(oc3c4c([2H])c([2H])c([2H])c([2H])c4c([2H])c([2H])c32)c1-c1nc(-c2cccc(-c3ccccc3)c2)nc(-c2ccc3c(c2)oc2cccc(-c4ccccc4)c23)n1] |
| 1-66 | 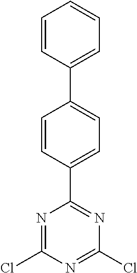 [SMILES: Clc1nc(Cl)nc(-c2ccc(-c3ccccc3)cc2)n1] | 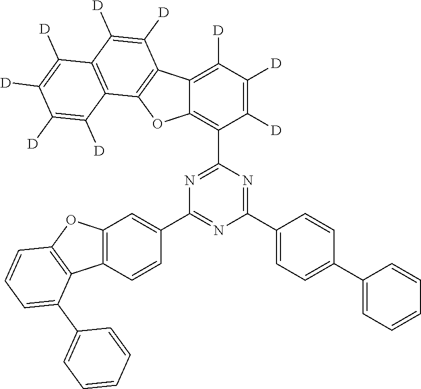 [SMILES: [2H]c1c([2H])c([2H])c2c(oc3c4c([2H])c([2H])c([2H])c([2H])c4c([2H])c([2H])c32)c1-c1nc(-c2ccc(-c3ccccc3)cc2)nc(-c2ccc3c(c2)oc2cccc(-c4ccccc4)c23)n1] |
| 1-67 | 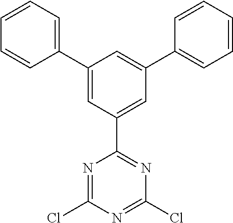 [SMILES: Clc1nc(Cl)nc(-c2cc(-c3ccccc3)cc(-c3ccccc3)c2)n1] | 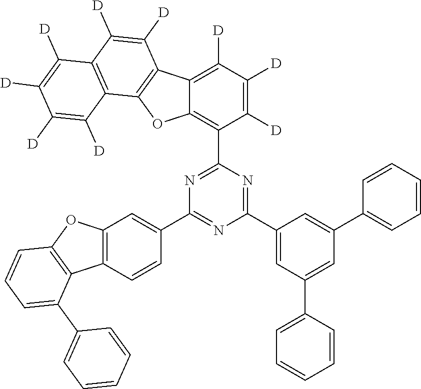 [SMILES: [2H]c1c([2H])c([2H])c2c(oc3c4c([2H])c([2H])c([2H])c([2H])c4c([2H])c([2H])c32)c1-c1nc(-c2cc(-c3ccccc3)cc(-c3ccccc3)c2)nc(-c2ccc3c(c2)oc2cccc(-c4ccccc4)c23)n1] |
| 1-68 | 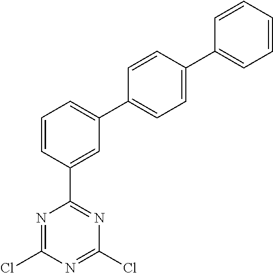 [SMILES: Clc1nc(Cl)nc(-c2cccc(-c3ccc(-c4ccccc4)cc3)c2)n1] | 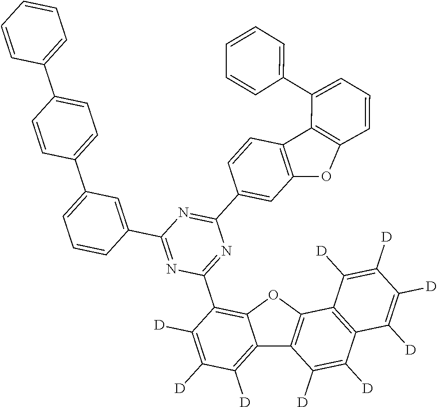 [SMILES: [2H]c1c([2H])c([2H])c2c(oc3c4c([2H])c([2H])c([2H])c([2H])c4c([2H])c([2H])c32)c1-c1nc(-c2cccc(-c3ccc(-c4ccccc4)cc3)c2)nc(-c2ccc3c(c2)oc2cccc(-c4ccccc4)c23)n1] |
| 1-69 | 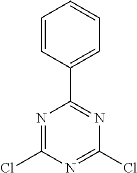 [SMILES: Clc1nc(Cl)nc(-c2ccccc2)n1] | 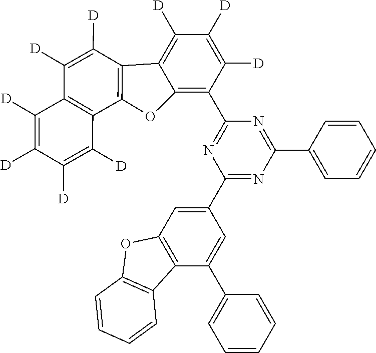 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)c3c(c2)oc2ccccc23)n1] |
| 1-70 | 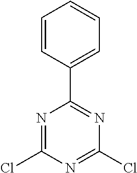 [SMILES: Clc1nc(Cl)nc(-c2ccccc2)n1] | 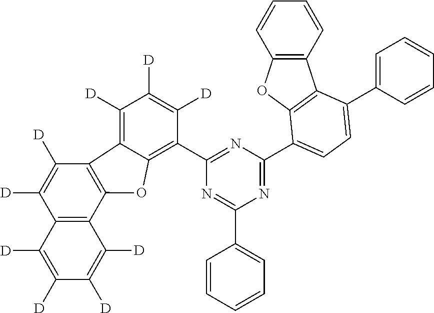 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3c2oc2ccccc23)n1] |
| 1-71 | 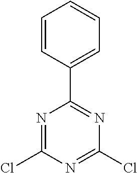 [SMILES: Clc1nc(Cl)nc(-c2ccccc2)n1] | 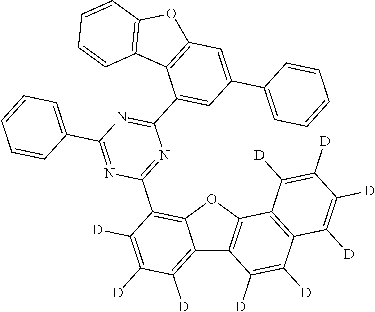 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)cc3oc4ccccc4c23)n1] |
| 1-72 | 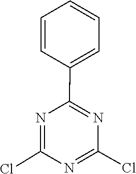 [SMILES: Clc1nc(Cl)nc(-c2ccccc2)n1] | 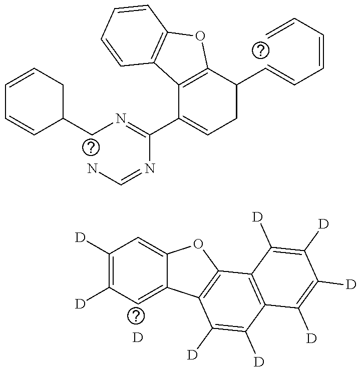 |
| 1-81 | 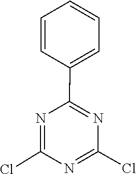 [SMILES: Clc1nc(Cl)nc(-c2ccccc2)n1] | 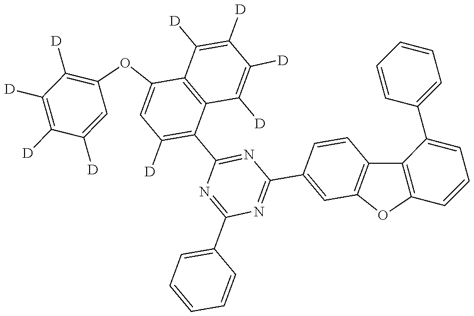 [SMILES: [2H]c1cc(Oc2cc([2H])c(-c3nc(-c4ccccc4)nc(-c4ccc5c(c4)oc4cccc(-c6ccccc6)c45)n3)c3c([2H])c([2H])c([2H])c([2H])c23)c([2H])c([2H])c1[2H]] |
| 1-82 | 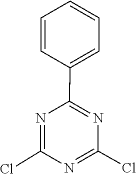 [SMILES: Clc1nc(Cl)nc(-c2ccccc2)n1] | 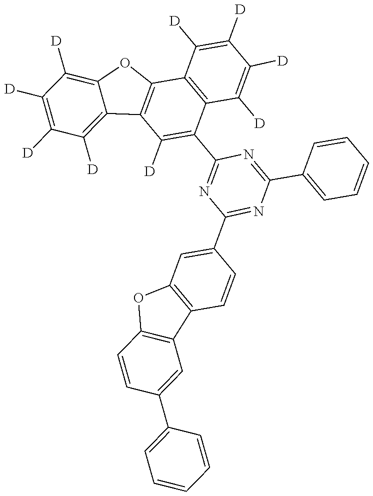 [SMILES: [2H]c1c([2H])c([2H])c2c(oc3c4c([2H])c([2H])c([2H])c([2H])c4c(-c4nc(-c5ccccc5)nc(-c5ccc6c(c5)oc5ccc(-c7ccccc7)cc56)n4)c([2H])c32)c1[2H]] |
| 1-83 | 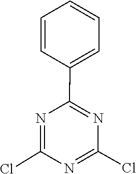 [SMILES: Clc1nc(Cl)nc(-c2ccccc2)n1] | 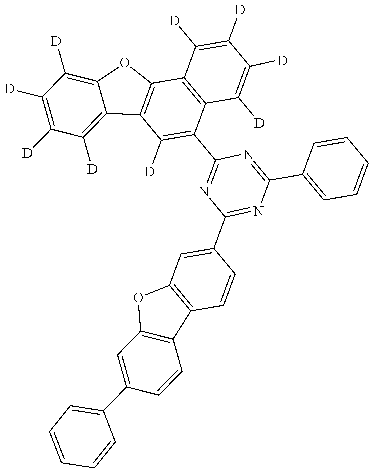 [SMILES: [2H]c1c([2H])c([2H])c2c(oc3c4c([2H])c([2H])c([2H])c([2H])c4c(-c4nc(-c5ccccc5)nc(-c5ccc6c(c5)oc5cc(-c7ccccc7)ccc56)n4)c([2H])c32)c1[2H]] |
| 1-84 | 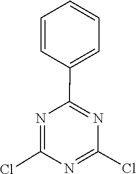 [SMILES: Clc1nc(Cl)nc(-c2ccccc2)n1] | 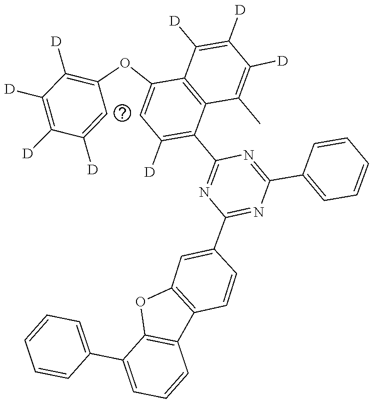 [SMILES: [2H]c1cc(Oc2cc([2H])c(-c3nc(-c4ccccc4)nc(-c4ccc5c(c4)oc4c(-c6ccccc6)cccc45)n3)c3c(C)c([2H])c([2H])c([2H])c23)c([2H])c([2H])c1[2H]] |
| 1-85 | 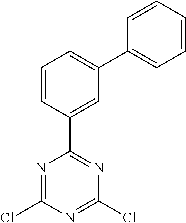 [SMILES: Clc1nc(Cl)nc(-c2cccc(-c3ccccc3)c2)n1] | 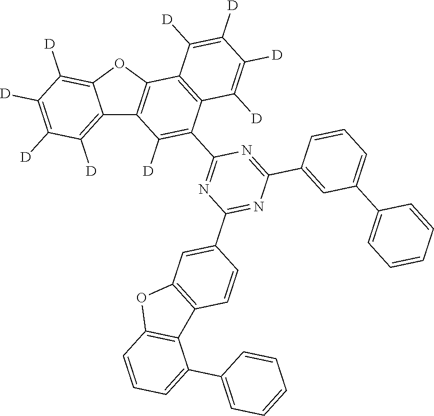 [SMILES: [2H]c1c([2H])c([2H])c2c(oc3c4c([2H])c([2H])c([2H])c([2H])c4c(-c4nc(-c5cccc(-c6ccccc6)c5)nc(-c5ccc6c(c5)oc5cccc(-c7ccccc7)c56)n4)c([2H])c32)c1[2H]] |
| 1-86 | 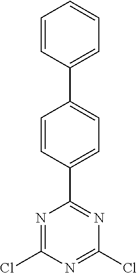 [SMILES: Clc1nc(Cl)nc(-c2ccc(-c3ccccc3)cc2)n1] | 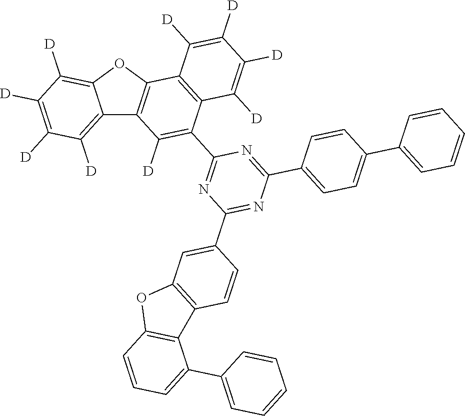 [SMILES: [2H]c1c([2H])c([2H])c2c(oc3c4c([2H])c([2H])c([2H])c([2H])c4c(-c4nc(-c5ccc(-c6ccccc6)cc5)nc(-c5ccc6c(c5)oc5cccc(-c7ccccc7)c56)n4)c([2H])c32)c1[2H]] |
| 1-87 | 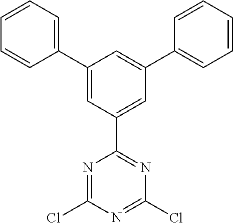 [SMILES: Clc1nc(Cl)nc(-c2cc(-c3ccccc3)cc(-c3ccccc3)c2)n1] | 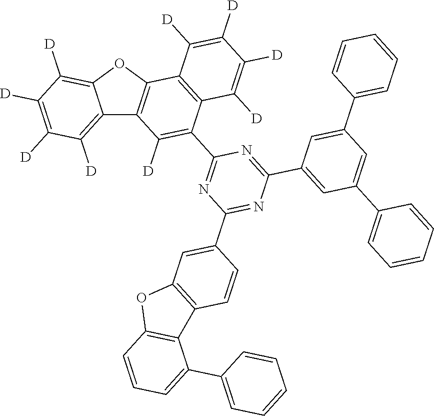 [SMILES: [2H]c1c([2H])c([2H])c2c(oc3c4c([2H])c([2H])c([2H])c([2H])c4c(-c4nc(-c5cc(-c6ccccc6)cc(-c6ccccc6)c5)nc(-c5ccc6c(c5)oc5cccc(-c7ccccc7)c56)n4)c([2H])c32)c1[2H]] |
| 1-88 | 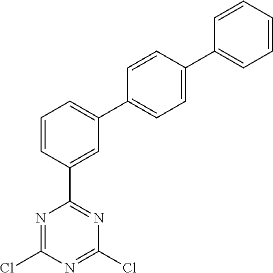 [SMILES: Clc1nc(Cl)nc(-c2cccc(-c3ccc(-c4ccccc4)cc3)c2)n1] | 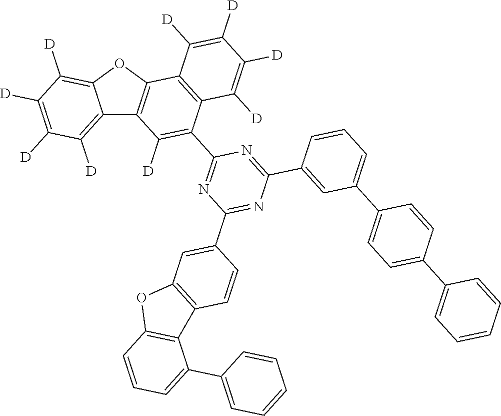 [SMILES: [2H]c1c([2H])c([2H])c2c(oc3c4c([2H])c([2H])c([2H])c([2H])c4c(-c4nc(-c5cccc(-c6ccc(-c7ccccc7)cc6)c5)nc(-c5ccc6c(c5)oc5cccc(-c7ccccc7)c56)n4)c([2H])c32)c1[2H]] |
| 1-89 | 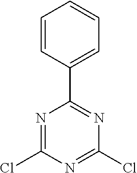 [SMILES: Clc1nc(Cl)nc(-c2ccccc2)n1] | 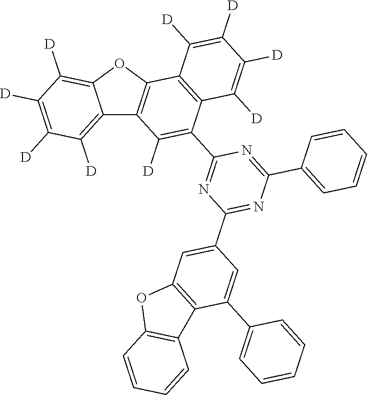 [SMILES: [2H]c1c([2H])c([2H])c2c(oc3c4c([2H])c([2H])c([2H])c([2H])c4c(-c4nc(-c5ccccc5)nc(-c5cc(-c6ccccc6)c6c(c5)oc5ccccc56)n4)c([2H])c32)c1[2H]] |
| 1-90 | 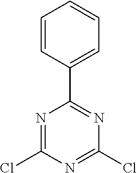 [SMILES: Clc1nc(Cl)nc(-c2ccccc2)n1] | 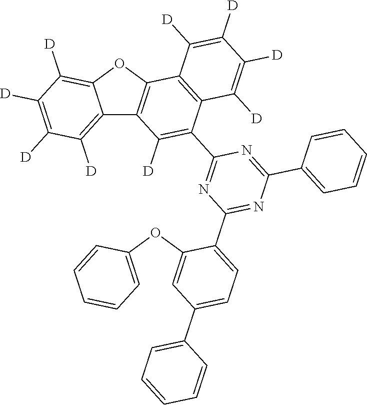 [SMILES: [2H]c1c([2H])c([2H])c2c(oc3c4c([2H])c([2H])c([2H])c([2H])c4c(-c4nc(-c5ccccc5)nc(-c5ccc(-c6ccccc6)cc5Oc5ccccc5)n4)c([2H])c32)c1[2H]] |
| 1-91 | 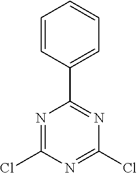 [SMILES: Clc1nc(Cl)nc(-c2ccccc2)n1] | 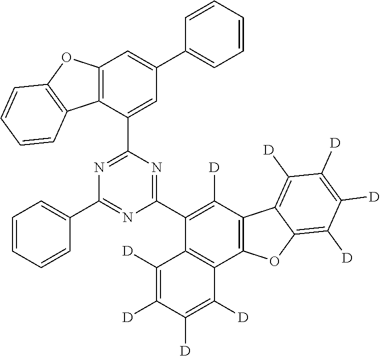 [SMILES: [2H]c1c([2H])c([2H])c2c(oc3c4c([2H])c([2H])c([2H])c([2H])c4c(-c4nc(-c5ccccc5)nc(-c5cc(-c6ccccc6)cc6oc7ccccc7c56)n4)c([2H])c32)c1[2H]] |
| 1-92 | 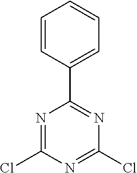 [SMILES: Clc1nc(Cl)nc(-c2ccccc2)n1] | 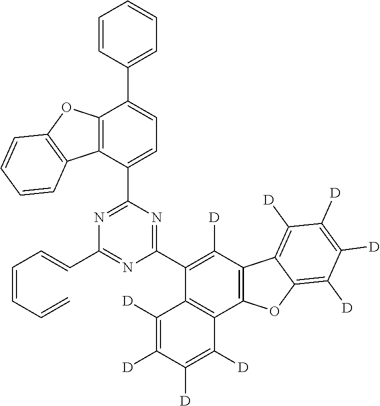 [SMILES: [2H]c1c([2H])c([2H])c2c(oc3c4c([2H])c([2H])c([2H])c([2H])c4c(-c4nc(/C=C/C=C\C=C)nc(-c5ccc(-c6ccccc6)c6oc7ccccc7c56)n4)c([2H])c32)c1[2H]] |
| 1-101 | 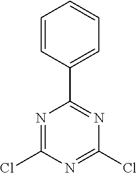 [SMILES: Clc1nc(Cl)nc(-c2ccccc2)n1] | 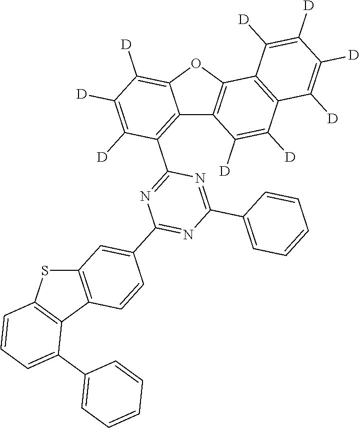 [SMILES: [2H]c1c([2H])c(-c2nc(-c3ccccc3)nc(-c3ccc4c(c3)sc3cccc(-c5ccccc5)c34)n2)c2c(oc3c4c([2H])c([2H])c([2H])c([2H])c4c([2H])c([2H])c32)c1[2H]] |
| 1-102 | 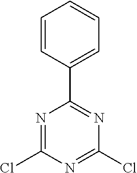 [SMILES: Clc1nc(Cl)nc(-c2ccccc2)n1] | 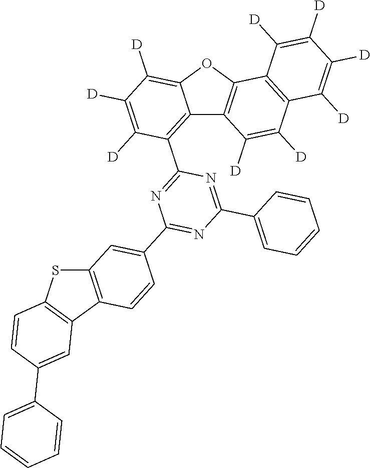 [SMILES: [2H]c1c([2H])c(-c2nc(-c3ccccc3)nc(-c3ccc4c(c3)sc3ccc(-c5ccccc5)cc34)n2)c2c(oc3c4c([2H])c([2H])c([2H])c([2H])c4c([2H])c([2H])c32)c1[2H]] |
| 1-103 | 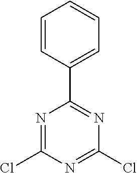 [SMILES: Clc1nc(Cl)nc(-c2ccccc2)n1] | 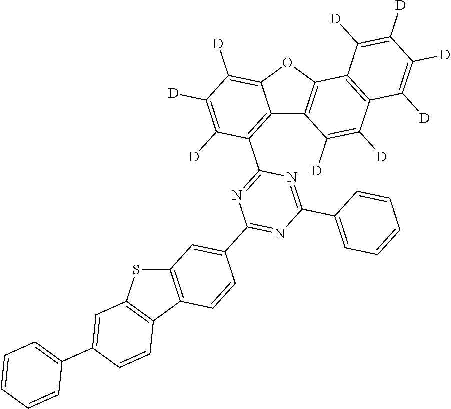 [SMILES: [2H]c1c([2H])c(-c2nc(-c3ccccc3)nc(-c3ccc4c(c3)sc3cc(-c5ccccc5)ccc34)n2)c2c(oc3c4c([2H])c([2H])c([2H])c([2H])c4c([2H])c([2H])c32)c1[2H]] |
| 1-104 | 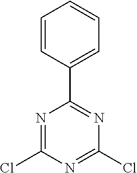 [SMILES: Clc1nc(Cl)nc(-c2ccccc2)n1] | 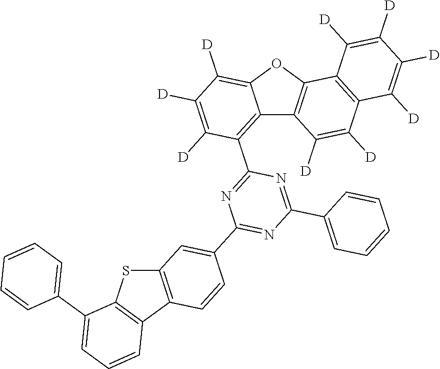 [SMILES: [2H]c1c([2H])c(-c2nc(-c3ccccc3)nc(-c3ccc4c(c3)sc3c(-c5ccccc5)cccc34)n2)c2c(oc3c4c([2H])c([2H])c([2H])c([2H])c4c([2H])c([2H])c32)c1[2H]] |
| 1-105 | 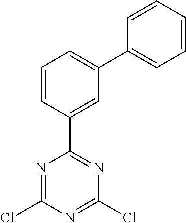 [SMILES: Clc1nc(Cl)nc(-c2cccc(-c3ccccc3)c2)n1] | 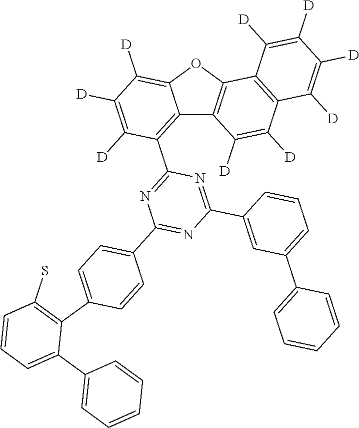 [SMILES: [2H]c1c([2H])c(-c2nc(-c3ccc(-c4c(S)cccc4-c4ccccc4)cc3)nc(-c3cccc(-c4ccccc4)c3)n2)c2c(oc3c4c([2H])c([2H])c([2H])c([2H])c4c([2H])c([2H])c32)c1[2H]] |
| 1-106 | 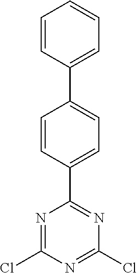 [SMILES: Clc1nc(Cl)nc(-c2ccc(-c3ccccc3)cc2)n1] | 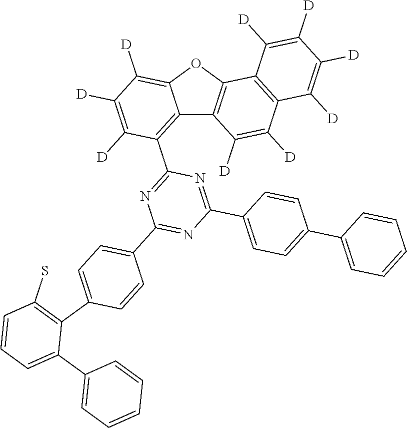 [SMILES: [2H]c1c([2H])c(-c2nc(-c3ccc(-c4ccccc4)cc3)nc(-c3ccc(-c4c(S)cccc4-c4ccccc4)cc3)n2)c2c(oc3c4c([2H])c([2H])c([2H])c([2H])c4c([2H])c([2H])c32)c1[2H]] |
| 1-107 | 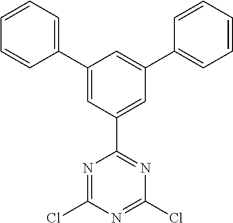 [SMILES: Clc1nc(Cl)nc(-c2cc(-c3ccccc3)cc(-c3ccccc3)c2)n1] | 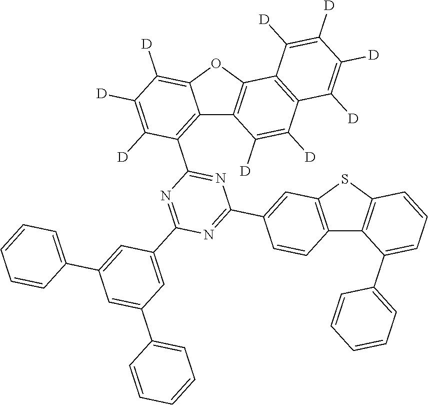 [SMILES: [2H]c1c([2H])c(-c2nc(-c3cc(-c4ccccc4)cc(-c4ccccc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H]] |
| 1-108 | 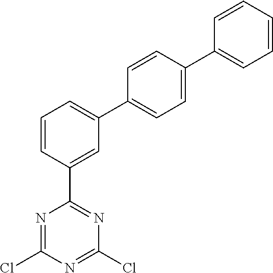 [SMILES: Clc1nc(Cl)nc(-c2cccc(-c3ccc(-c4ccccc4)cc3)c2)n1] | 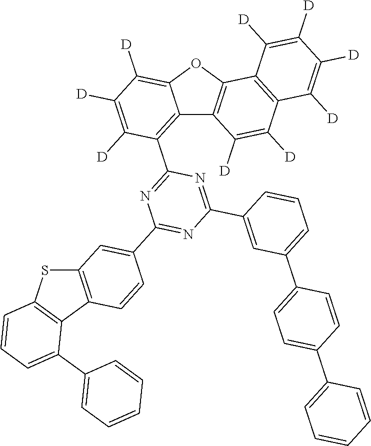 [SMILES: [2H]c1c([2H])c(-c2nc(-c3cccc(-c4ccc(-c5ccccc5)cc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H]] |
| 1-109 | 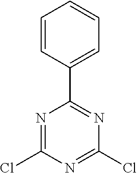 [SMILES: Clc1nc(Cl)nc(-c2ccccc2)n1] | 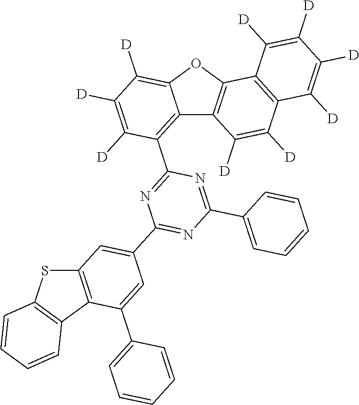 [SMILES: [2H]c1c([2H])c(-c2nc(-c3ccccc3)nc(-c3cc(-c4ccccc4)c4c(c3)sc3ccccc34)n2)c2c(oc3c4c([2H])c([2H])c([2H])c([2H])c4c([2H])c([2H])c32)c1[2H]] |
| 1-110 | 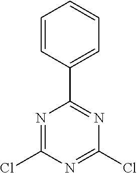 [SMILES: Clc1nc(Cl)nc(-c2ccccc2)n1] | 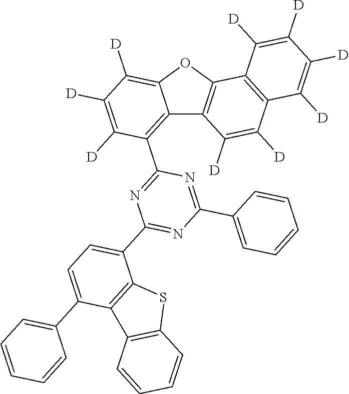 [SMILES: [2H]c1c([2H])c(-c2nc(-c3ccccc3)nc(-c3ccc(-c4ccccc4)c4c3sc3ccccc34)n2)c2c(oc3c4c([2H])c([2H])c([2H])c([2H])c4c([2H])c([2H])c32)c1[2H]] |
| 1-111 | 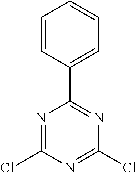 [SMILES: Clc1nc(Cl)nc(-c2ccccc2)n1] | 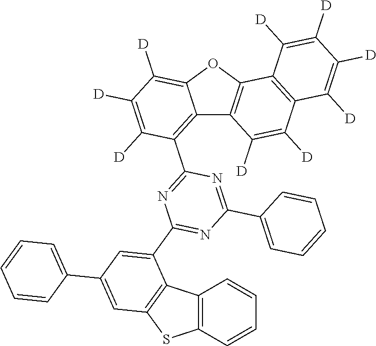 [SMILES: [2H]c1c([2H])c(-c2nc(-c3ccccc3)nc(-c3cc(-c4ccccc4)cc4sc5ccccc5c34)n2)c2c(oc3c4c([2H])c([2H])c([2H])c([2H])c4c([2H])c([2H])c32)c1[2H]] |
| 1-112 | 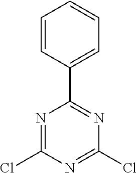 [SMILES: Clc1nc(Cl)nc(-c2ccccc2)n1] | 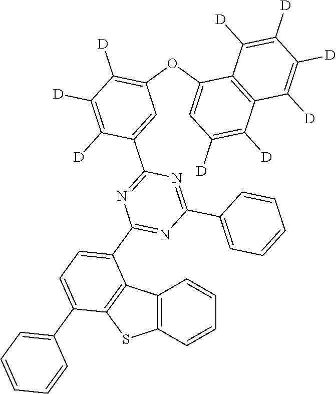 [SMILES: [2H]c1cc(Oc2cc(-c3nc(-c4ccccc4)nc(-c4ccc(-c5ccccc5)c5sc6ccccc6c45)n3)c([2H])c([2H])c2[2H])c2c([2H])c([2H])c([2H])c([2H])c2c1[2H]] |
| 1-121 | 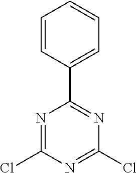 [SMILES: Clc1nc(Cl)nc(-c2ccccc2)n1] | 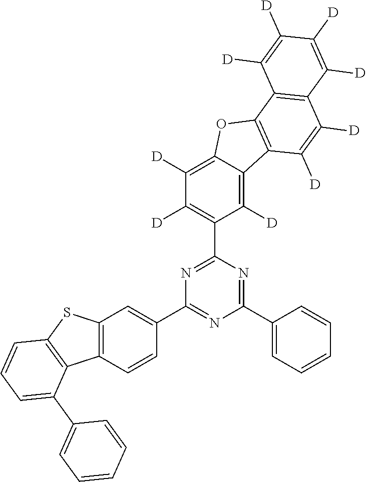 [SMILES: [2H]c1c(-c2nc(-c3ccccc3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]] |
| 1-122 | 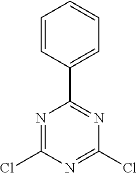 [SMILES: Clc1nc(Cl)nc(-c2ccccc2)n1] | 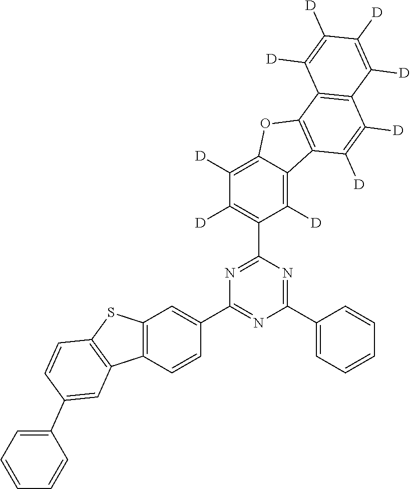 [SMILES: [2H]c1c(-c2nc(-c3ccccc3)nc(-c3ccc4c(c3)sc3ccc(-c5ccccc5)cc34)n2)c([2H])c2c(oc3c4c([2H])c([2H])c([2H])c([2H])c4c([2H])c([2H])c32)c1[2H]] |
| 1-123 | 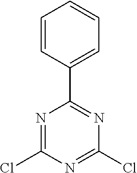 [SMILES: Clc1nc(Cl)nc(-c2ccccc2)n1] | 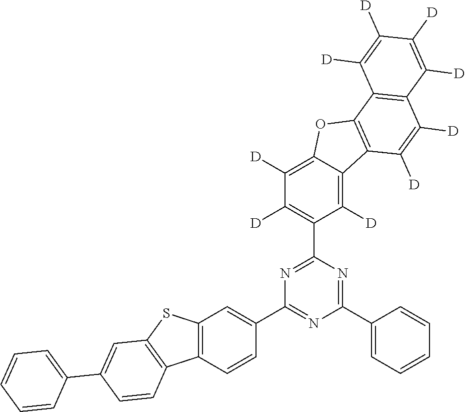 [SMILES: [2H]c1c(-c2nc(-c3ccccc3)nc(-c3ccc4c(c3)sc3cc(-c5ccccc5)ccc34)n2)c([2H])c2c(oc3c4c([2H])c([2H])c([2H])c([2H])c4c([2H])c([2H])c32)c1[2H]] |
| 1-124 | 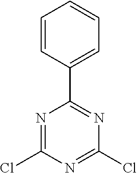 [SMILES: Clc1nc(Cl)nc(-c2ccccc2)n1] | 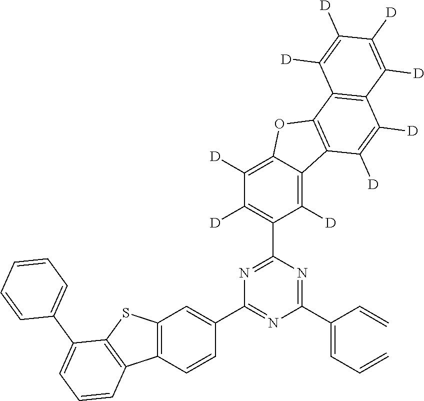 [SMILES: [2H]c1c(-c2nc(/C(C=C)=C/C=C)nc(-c3ccc4c(c3)sc3c(-c5ccccc5)cccc34)n2)c([2H])c2c(oc3c4c([2H])c([2H])c([2H])c([2H])c4c([2H])c([2H])c32)c1[2H]] |
| 1-125 | 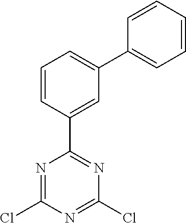 [SMILES: Clc1nc(Cl)nc(-c2cccc(-c3ccccc3)c2)n1] | 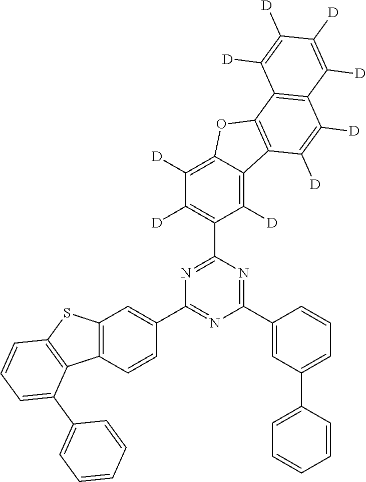 [SMILES: [2H]c1c(-c2nc(-c3cccc(-c4ccccc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]] |
| 1-126 | 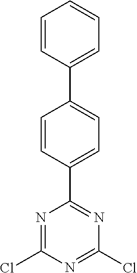 [SMILES: Clc1nc(Cl)nc(-c2ccc(-c3ccccc3)cc2)n1] | 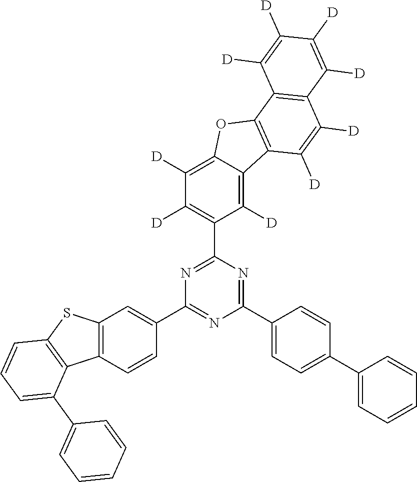 [SMILES: [2H]c1c(-c2nc(-c3ccc(-c4ccccc4)cc3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]] |
| 1-127 | 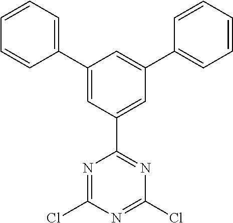 [SMILES: Clc1nc(Cl)nc(-c2cc(-c3ccccc3)cc(-c3ccccc3)c2)n1] | 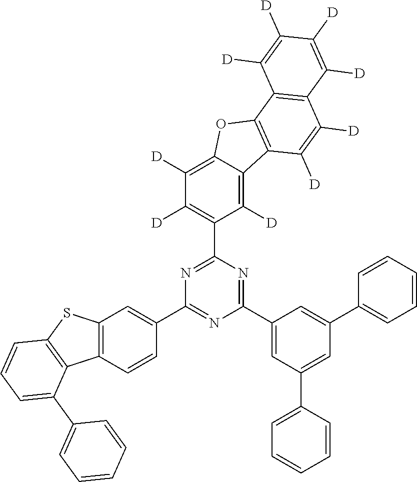 [SMILES: [2H]c1c(-c2nc(-c3cc(-c4ccccc4)cc(-c4ccccc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]] |
| 1-128 | 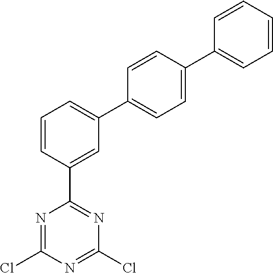 [SMILES: Clc1nc(Cl)nc(-c2cccc(-c3ccc(-c4ccccc4)cc3)c2)n1] | 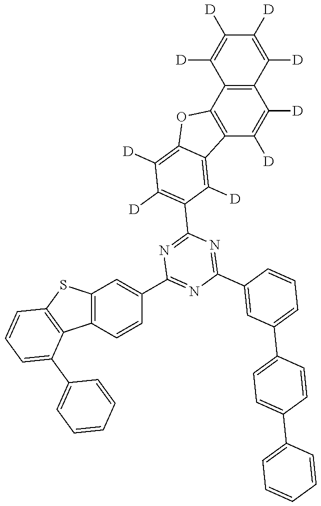 [SMILES: [2H]c1c(-c2nc(-c3cccc(-c4ccc(-c5ccccc5)cc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]] |
| 1-129 | 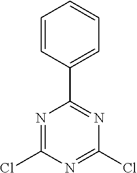 [SMILES: Clc1nc(Cl)nc(-c2ccccc2)n1] | 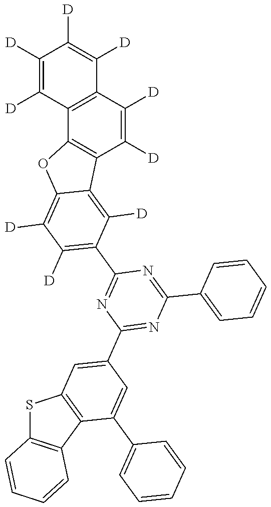 [SMILES: [2H]c1c(-c2nc(-c3ccccc3)nc(-c3cc(-c4ccccc4)c4c(c3)sc3ccccc34)n2)c([2H])c2c(oc3c4c([2H])c([2H])c([2H])c([2H])c4c([2H])c([2H])c32)c1[2H]] |
| 1-130 | 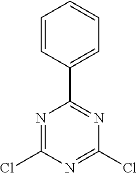 [SMILES: Clc1nc(Cl)nc(-c2ccccc2)n1] | 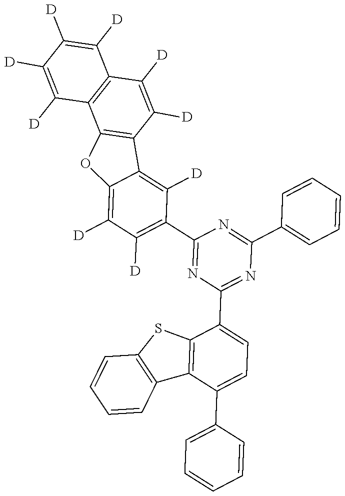 [SMILES: [2H]c1c(-c2nc(-c3ccccc3)nc(-c3ccc(-c4ccccc4)c4c3sc3ccccc34)n2)c([2H])c2c(oc3c4c([2H])c([2H])c([2H])c([2H])c4c([2H])c([2H])c32)c1[2H]] |
| 1-131 | 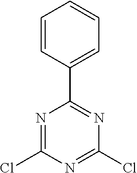 [SMILES: Clc1nc(Cl)nc(-c2ccccc2)n1] | 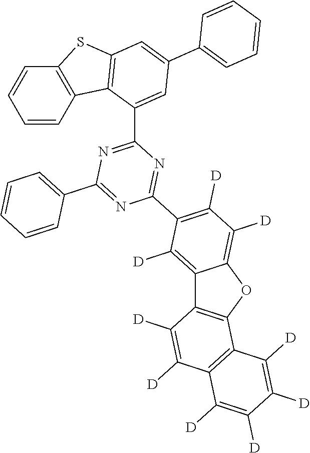 [SMILES: [2H]c1c(-c2nc(-c3ccccc3)nc(-c3cc(-c4ccccc4)cc4sc5ccccc5c34)n2)c([2H])c2c(oc3c4c([2H])c([2H])c([2H])c([2H])c4c([2H])c([2H])c32)c1[2H]] |
| 1-132 | 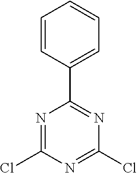 [SMILES: Clc1nc(Cl)nc(-c2ccccc2)n1] | 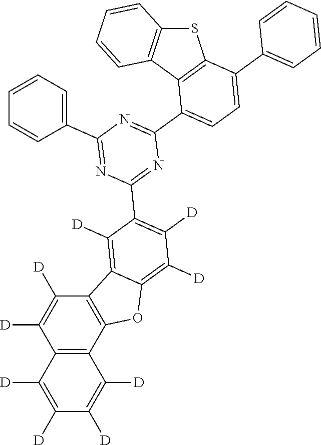 [SMILES: [2H]c1c(-c2nc(-c3ccccc3)nc(-c3ccc(-c4ccccc4)c4sc5ccccc5c34)n2)c([2H])c2c(oc3c4c([2H])c([2H])c([2H])c([2H])c4c([2H])c([2H])c32)c1[2H]] |
| 1-141 | 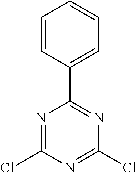 [SMILES: Clc1nc(Cl)nc(-c2ccccc2)n1] | 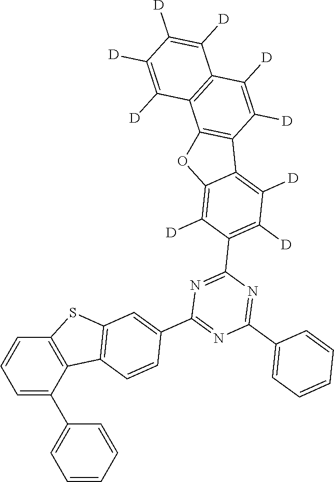 [SMILES: [2H]c1c([2H])c([2H])c2c(c1[2H])c([2H])c([2H])c1c2oc2c([2H])c(-c3nc(-c4ccccc4)nc(-c4ccc5c(c4)sc4cccc(-c6ccccc6)c45)n3)c([2H])c([2H])c21] |
| 1-142 | 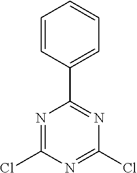 [SMILES: Clc1nc(Cl)nc(-c2ccccc2)n1] | 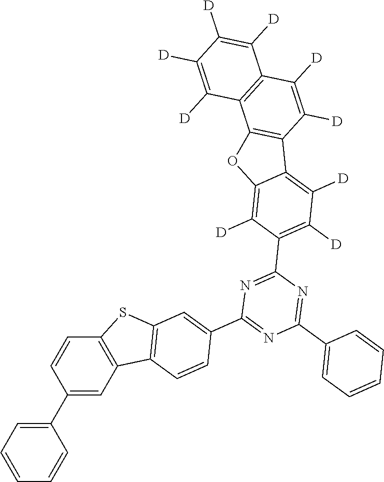 [SMILES: [2H]c1c([2H])c([2H])c2c(c1[2H])c([2H])c([2H])c1c2oc2c([2H])c(-c3nc(-c4ccccc4)nc(-c4ccc5c(c4)sc4ccc(-c6ccccc6)cc45)n3)c([2H])c([2H])c21] |
| 1-143 | 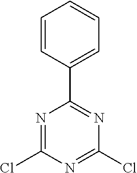 [SMILES: Clc1nc(Cl)nc(-c2ccccc2)n1] | 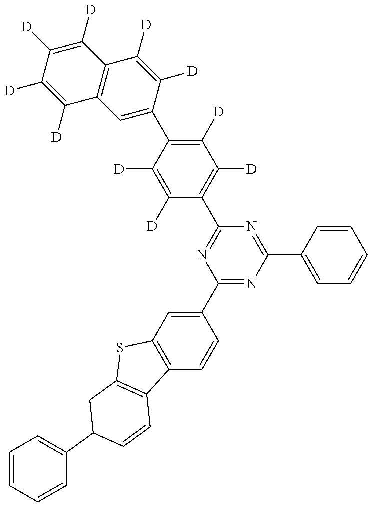 [SMILES: [2H]c1c([2H])c(-c2cc3c([2H])c([2H])c([2H])c([2H])c3c([2H])c2[2H])c([2H])c([2H])c1-c1nc(-c2ccccc2)nc(-c2ccc3c4c(sc3c2)CC(c2ccccc2)C=C4)n1] |
| 1-144 | 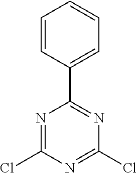 [SMILES: Clc1nc(Cl)nc(-c2ccccc2)n1] | 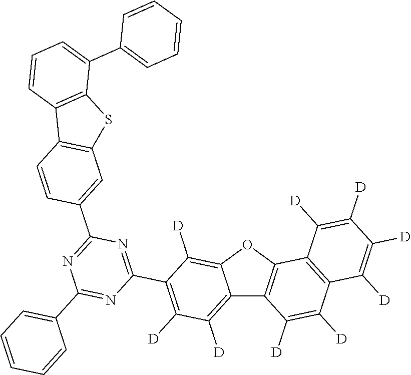 [SMILES: [2H]c1c([2H])c([2H])c2c(c1[2H])c([2H])c([2H])c1c2oc2c([2H])c(-c3nc(-c4ccccc4)nc(-c4ccc5c(c4)sc4c(-c6ccccc6)cccc45)n3)c([2H])c([2H])c21] |
| 1-145 | 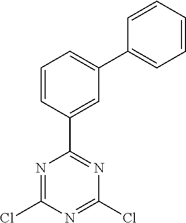 [SMILES: Clc1nc(Cl)nc(-c2cccc(-c3ccccc3)c2)n1] | 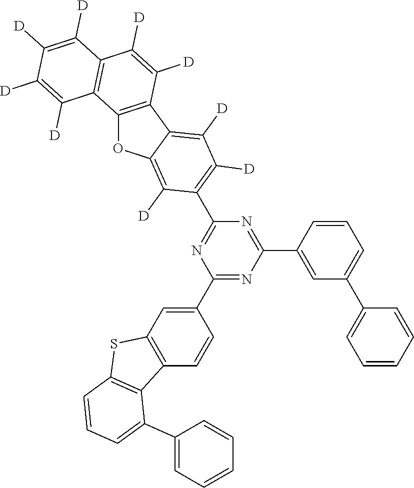 [SMILES: [2H]c1c([2H])c([2H])c2c(c1[2H])c([2H])c([2H])c1c2oc2c([2H])c(-c3nc(-c4cccc(-c5ccccc5)c4)nc(-c4ccc5c(c4)sc4cccc(-c6ccccc6)c45)n3)c([2H])c([2H])c21] |
| 1-146 | 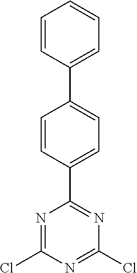 [SMILES: Clc1nc(Cl)nc(-c2ccc(-c3ccccc3)cc2)n1] | 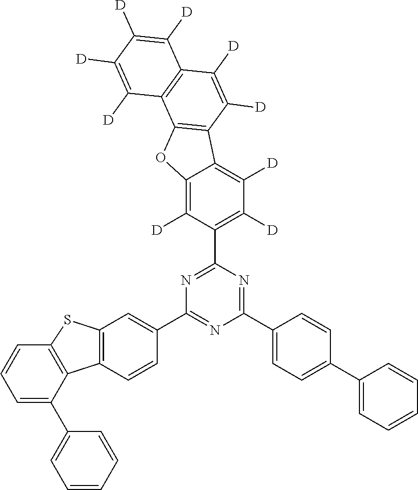 [SMILES: [2H]c1c([2H])c([2H])c2c(c1[2H])c([2H])c([2H])c1c2oc2c([2H])c(-c3nc(-c4ccc(-c5ccccc5)cc4)nc(-c4ccc5c(c4)sc4cccc(-c6ccccc6)c45)n3)c([2H])c([2H])c21] |
| 1-147 | 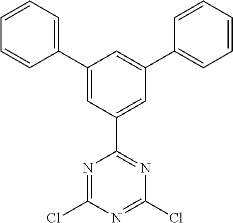 [SMILES: Clc1nc(Cl)nc(-c2cc(-c3ccccc3)cc(-c3ccccc3)c2)n1] | 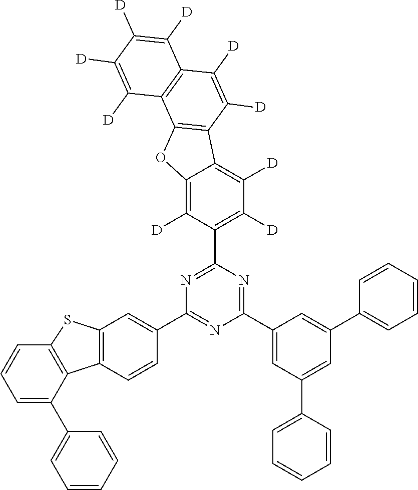 [SMILES: [2H]c1c([2H])c([2H])c2c(c1[2H])c([2H])c([2H])c1c2oc2c([2H])c(-c3nc(-c4cc(-c5ccccc5)cc(-c5ccccc5)c4)nc(-c4ccc5c(c4)sc4cccc(-c6ccccc6)c45)n3)c([2H])c([2H])c21] |
| 1-148 | 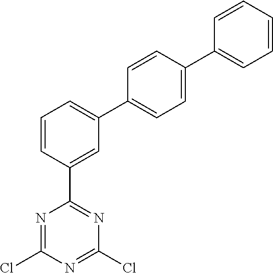 [SMILES: Clc1nc(Cl)nc(-c2cccc(-c3ccc(-c4ccccc4)cc3)c2)n1] | 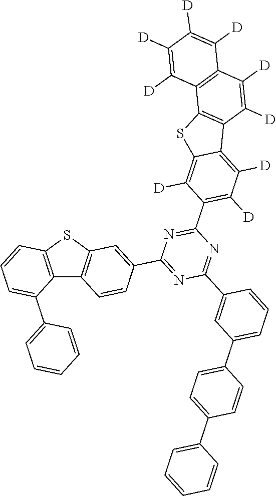 [SMILES: [2H]c1c([2H])c([2H])c2c(c1[2H])c([2H])c([2H])c1c2sc2c([2H])c(-c3nc(-c4cccc(-c5ccc(-c6ccccc6)cc5)c4)nc(-c4ccc5c(c4)sc4cccc(-c6ccccc6)c45)n3)c([2H])c([2H])c21] |
| 1-149 | 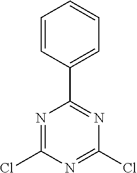 [SMILES: Clc1nc(Cl)nc(-c2ccccc2)n1] | 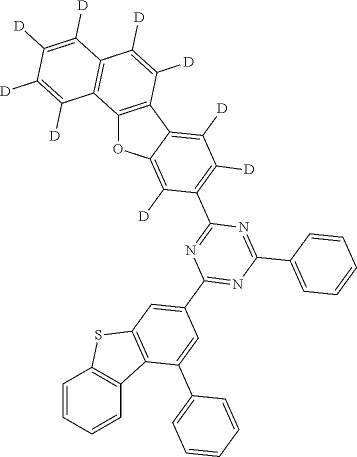 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)c5c(c4)sc4ccccc45)n3)c([2H])c([2H])c21] |
| 1-150 | 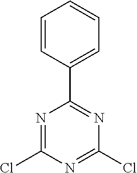 [SMILES: Clc1nc(Cl)nc(-c2ccccc2)n1] | 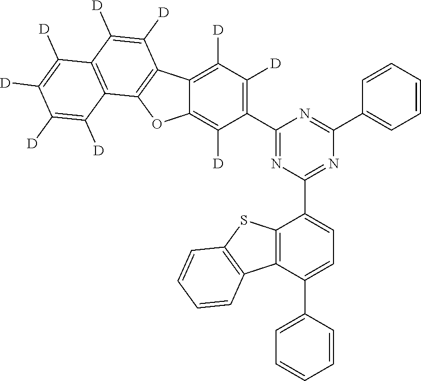 [SMILES: [2H]c1c([2H])c([2H])c2c(c1[2H])c([2H])c([2H])c1c2oc2c([2H])c(-c3nc(-c4ccccc4)nc(-c4ccc(-c5ccccc5)c5c4sc4ccccc45)n3)c([2H])c([2H])c21] |
| 1-151 | 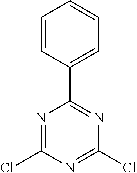 [SMILES: Clc1nc(Cl)nc(-c2ccccc2)n1] | 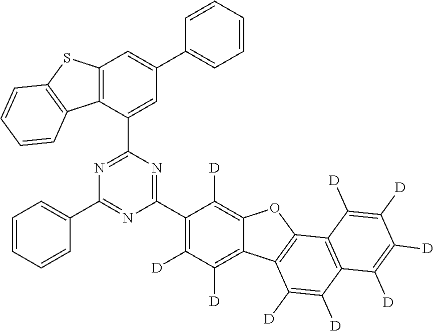 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)cc5sc6ccccc6c45)n3)c([2H])c([2H])c21] |
| 1-152 | 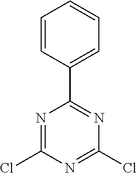 [SMILES: Clc1nc(Cl)nc(-c2ccccc2)n1] | 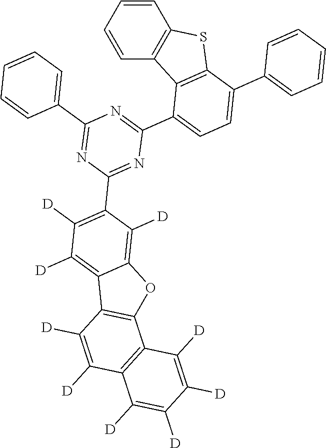 [SMILES: [2H]c1c([2H])c([2H])c2c(c1[2H])c([2H])c([2H])c1c2oc2c([2H])c(-c3nc(-c4ccccc4)nc(-c4ccc(-c5ccccc5)c5sc6ccccc6c45)n3)c([2H])c([2H])c21] |
| 1-161 | 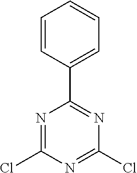 [SMILES: Clc1nc(Cl)nc(-c2ccccc2)n1] | 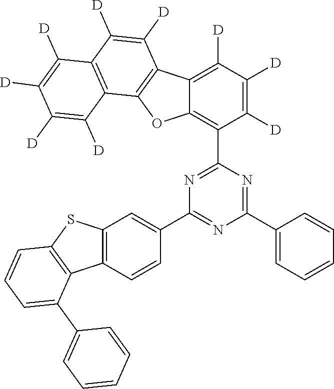 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2cccc(-c4ccccc4)c23)n1] |
| 1-162 | 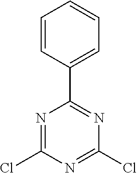 [SMILES: Clc1nc(Cl)nc(-c2ccccc2)n1] | 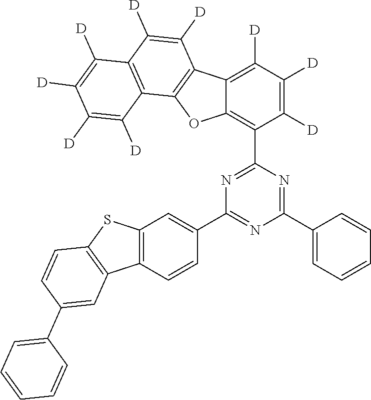 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2ccc(-c4ccccc4)cc23)n1] |
| 1-163 | 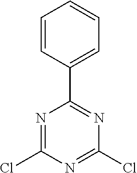 [SMILES: Clc1nc(Cl)nc(-c2ccccc2)n1] | 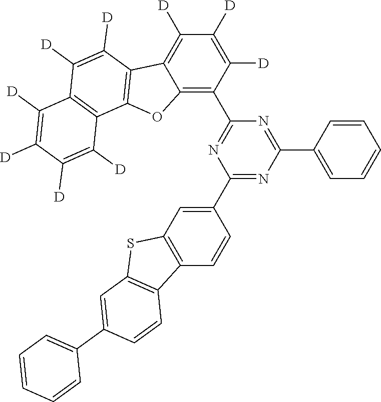 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2cc(-c4ccccc4)ccc23)n1] |
| 1-164 | 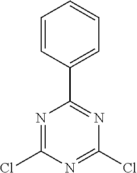 [SMILES: Clc1nc(Cl)nc(-c2ccccc2)n1] | 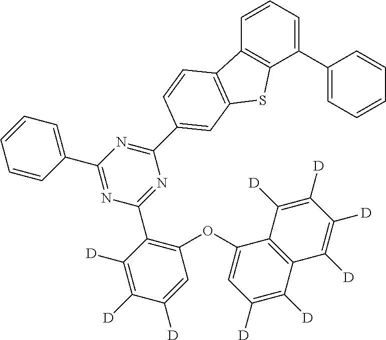 [SMILES: [2H]c1cc(Oc2cc([2H])c([2H])c3c([2H])c([2H])c([2H])c([2H])c23)c(-c2nc(-c3ccccc3)nc(-c3ccc4c(c3)sc3c(-c5ccccc5)cccc34)n2)c([2H])c1[2H]] |
| 1-165 | 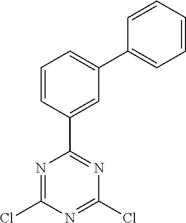 [SMILES: Clc1nc(Cl)nc(-c2cccc(-c3ccccc3)c2)n1] | 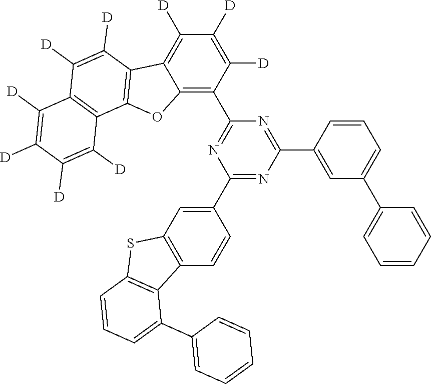 [SMILES: [2H]c1c([2H])c([2H])c2c(oc3c4c([2H])c([2H])c([2H])c([2H])c4c([2H])c([2H])c32)c1-c1nc(-c2cccc(-c3ccccc3)c2)nc(-c2ccc3c(c2)sc2cccc(-c4ccccc4)c23)n1] |
| 1-166 | 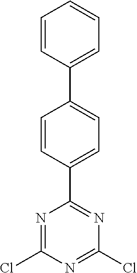 [SMILES: Clc1nc(Cl)nc(-c2ccc(-c3ccccc3)cc2)n1] | 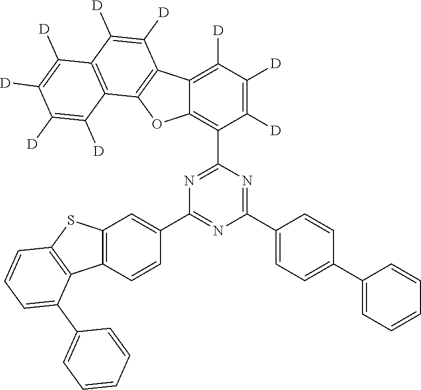 [SMILES: [2H]c1c([2H])c([2H])c2c(oc3c4c([2H])c([2H])c([2H])c([2H])c4c([2H])c([2H])c32)c1-c1nc(-c2ccc(-c3ccccc3)cc2)nc(-c2ccc3c(c2)sc2cccc(-c4ccccc4)c23)n1] |
| 1-167 | 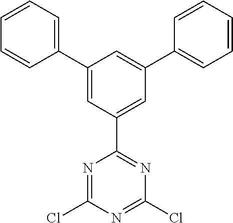 [SMILES: Clc1nc(Cl)nc(-c2cc(-c3ccccc3)cc(-c3ccccc3)c2)n1] | 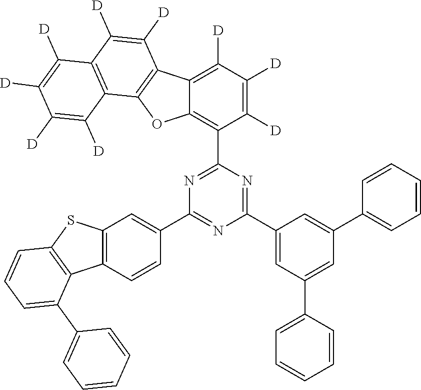 [SMILES: [2H]c1c([2H])c([2H])c2c(oc3c4c([2H])c([2H])c([2H])c([2H])c4c([2H])c([2H])c32)c1-c1nc(-c2cc(-c3ccccc3)cc(-c3ccccc3)c2)nc(-c2ccc3c(c2)sc2cccc(-c4ccccc4)c23)n1] |
| 1-168 | 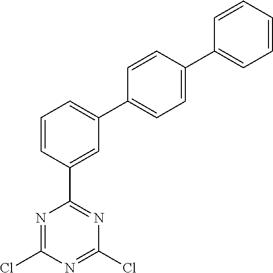 [SMILES: Clc1nc(Cl)nc(-c2cccc(-c3ccc(-c4ccccc4)cc3)c2)n1] | 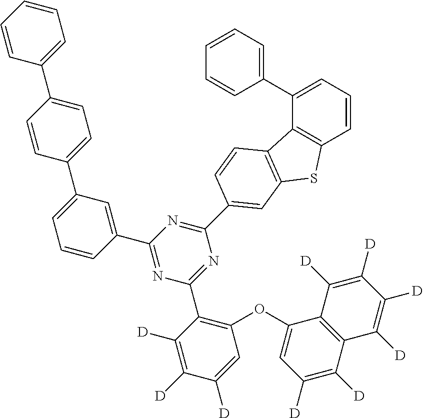 [SMILES: [2H]c1cc(Oc2cc([2H])c([2H])c3c([2H])c([2H])c([2H])c([2H])c23)c(-c2nc(-c3cccc(-c4ccc(-c5ccccc5)cc4)c3)nc(-c3ccc4c(c3)sc3cccc(-c5ccccc5)c34)n2)c([2H])c1[2H]] |
| 1-169 | 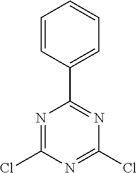 [SMILES: Clc1nc(Cl)nc(-c2ccccc2)n1] | 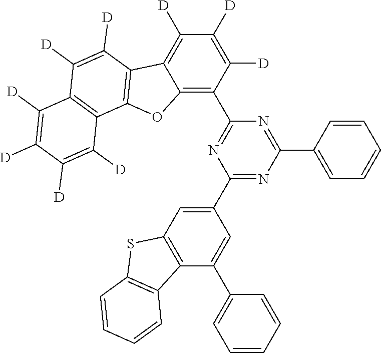 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)c3c(c2)sc2ccccc23)n1] |
| 1-170 | 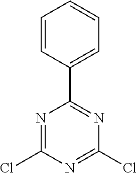 [SMILES: Clc1nc(Cl)nc(-c2ccccc2)n1] | 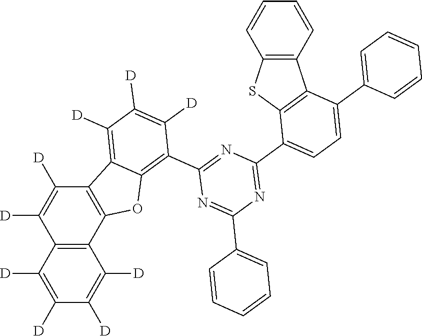 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3c2sc2ccccc23)n1] |
| 1-171 | 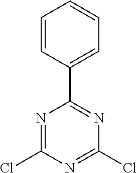 [SMILES: Clc1nc(Cl)nc(-c2ccccc2)n1] | 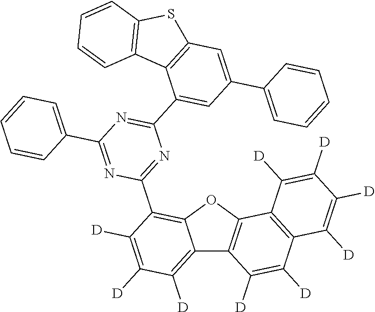 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)cc3sc4ccccc4c23)n1] |
| 1-172 | 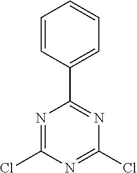 [SMILES: Clc1nc(Cl)nc(-c2ccccc2)n1] | 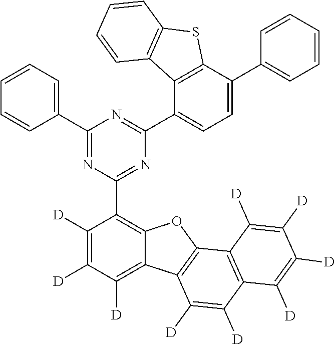 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3sc4ccccc4c23)n1] |
| 1-181 | 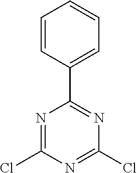 [SMILES: Clc1nc(Cl)nc(-c2ccccc2)n1] | 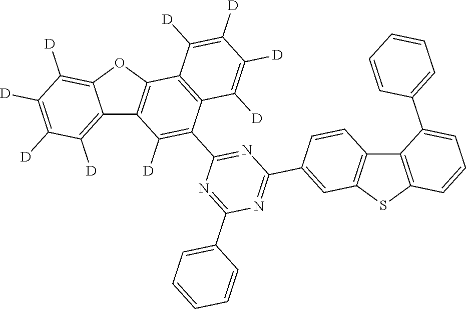 [SMILES: [2H]c1c([2H])c([2H])c2c(oc3c4c([2H])c([2H])c([2H])c([2H])c4c(-c4nc(-c5ccccc5)nc(-c5ccc6c(c5)sc5cccc(-c7ccccc7)c56)n4)c([2H])c32)c1[2H]] |
| 1-182 | 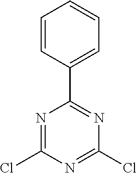 [SMILES: Clc1nc(Cl)nc(-c2ccccc2)n1] | 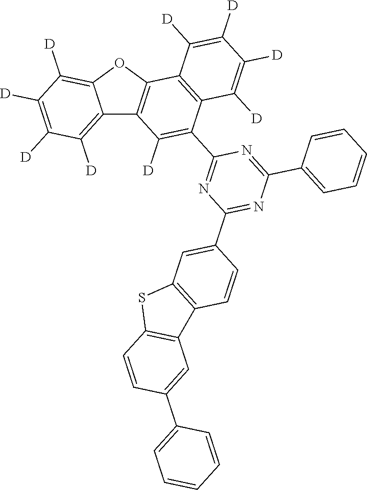 [SMILES: [2H]c1c([2H])c([2H])c2c(oc3c4c([2H])c([2H])c([2H])c([2H])c4c(-c4nc(-c5ccccc5)nc(-c5ccc6c(c5)sc5ccc(-c7ccccc7)cc56)n4)c([2H])c32)c1[2H]] |
| 1-183 | 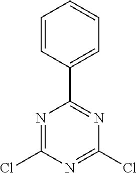 [SMILES: Clc1nc(Cl)nc(-c2ccccc2)n1] | 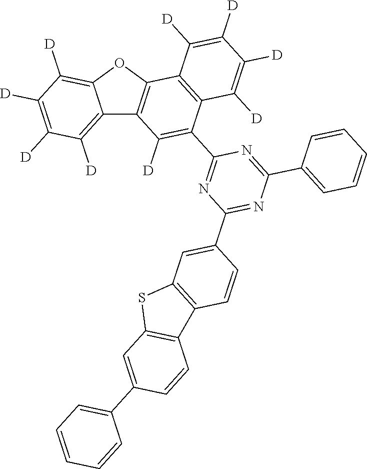 [SMILES: [2H]c1c([2H])c([2H])c2c(oc3c4c([2H])c([2H])c([2H])c([2H])c4c(-c4nc(-c5ccccc5)nc(-c5ccc6c(c5)sc5cc(-c7ccccc7)ccc56)n4)c([2H])c32)c1[2H]] |
| 1-184 | 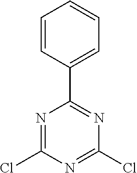 [SMILES: Clc1nc(Cl)nc(-c2ccccc2)n1] | 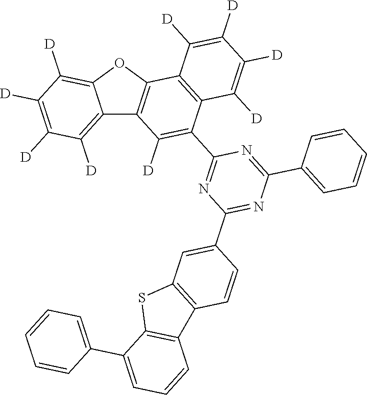 [SMILES: [2H]c1c([2H])c([2H])c2c(oc3c4c([2H])c([2H])c([2H])c([2H])c4c(-c4nc(-c5ccccc5)nc(-c5ccc6c(c5)sc5c(-c7ccccc7)cccc56)n4)c([2H])c32)c1[2H]] |
| 1-185 | 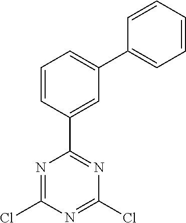 [SMILES: Clc1nc(Cl)nc(-c2cccc(-c3ccccc3)c2)n1] | 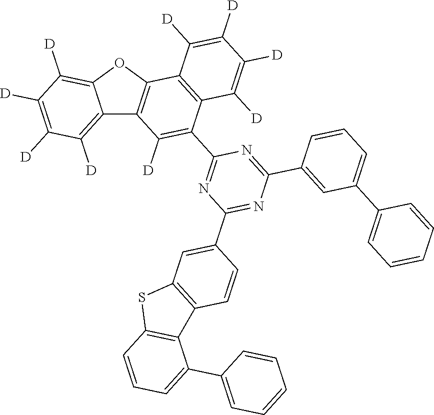 [SMILES: [2H]c1c([2H])c([2H])c2c(oc3c4c([2H])c([2H])c([2H])c([2H])c4c(-c4nc(-c5cccc(-c6ccccc6)c5)nc(-c5ccc6c(c5)sc5cccc(-c7ccccc7)c56)n4)c([2H])c32)c1[2H]] |
| 1-186 | 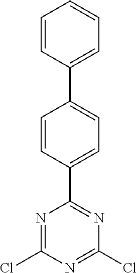 [SMILES: Clc1nc(Cl)nc(-c2ccc(-c3ccccc3)cc2)n1] | 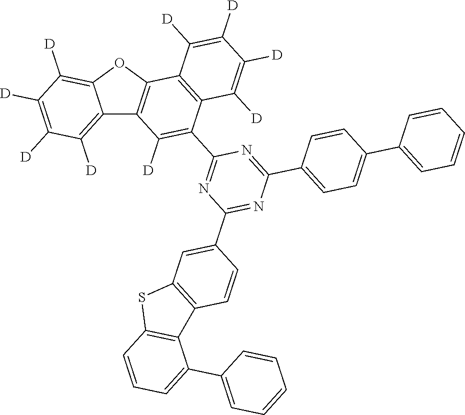 [SMILES: [2H]c1c([2H])c([2H])c2c(oc3c4c([2H])c([2H])c([2H])c([2H])c4c(-c4nc(-c5ccc(-c6ccccc6)cc5)nc(-c5ccc6c(c5)sc5cccc(-c7ccccc7)c56)n4)c([2H])c32)c1[2H]] |
| 1-187 | 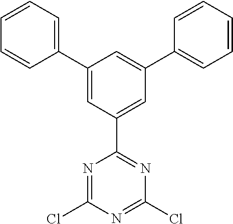 [SMILES: Clc1nc(Cl)nc(-c2cc(-c3ccccc3)cc(-c3ccccc3)c2)n1] | 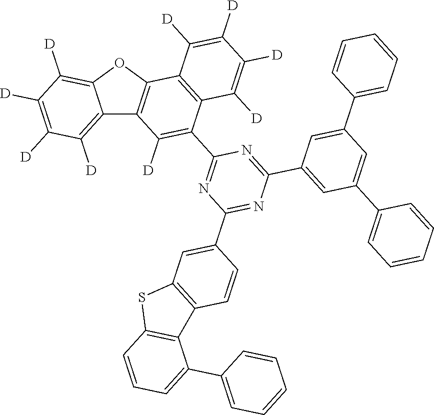 [SMILES: [2H]c1c([2H])c([2H])c2c(oc3c4c([2H])c([2H])c([2H])c([2H])c4c(-c4nc(-c5cc(-c6ccccc6)cc(-c6ccccc6)c5)nc(-c5ccc6c(c5)sc5cccc(-c7ccccc7)c56)n4)c([2H])c32)c1[2H]] |
| 1-188 | 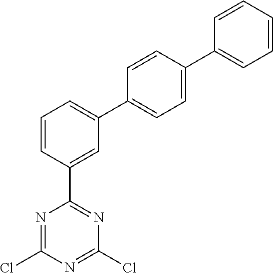 [SMILES: Clc1nc(Cl)nc(-c2cccc(-c3ccc(-c4ccccc4)cc3)c2)n1] | 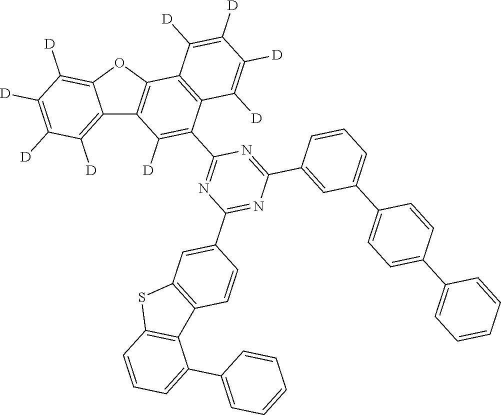 [SMILES: [2H]c1c([2H])c([2H])c2c(oc3c4c([2H])c([2H])c([2H])c([2H])c4c(-c4nc(-c5cccc(-c6ccc(-c7ccccc7)cc6)c5)nc(-c5ccc6c(c5)sc5cccc(-c7ccccc7)c56)n4)c([2H])c32)c1[2H]] |
| 1-189 | 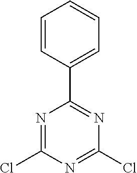 [SMILES: Clc1nc(Cl)nc(-c2ccccc2)n1] | 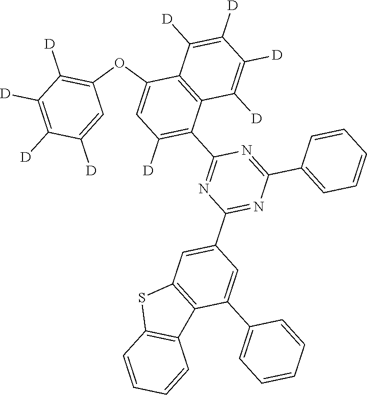 [SMILES: [2H]c1cc(Oc2cc([2H])c(-c3nc(-c4ccccc4)nc(-c4cc(-c5ccccc5)c5c(c4)sc4ccccc45)n3)c3c([2H])c([2H])c([2H])c([2H])c23)c([2H])c([2H])c1[2H]] |
| 1-190 | 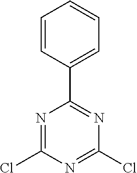 [SMILES: Clc1nc(Cl)nc(-c2ccccc2)n1] | 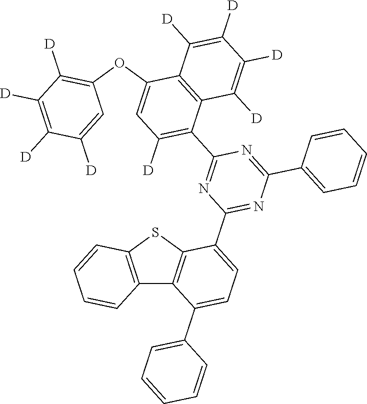 [SMILES: [2H]c1cc(Oc2cc([2H])c(-c3nc(-c4ccccc4)nc(-c4ccc(-c5ccccc5)c5c4sc4ccccc45)n3)c3c([2H])c([2H])c([2H])c([2H])c23)c([2H])c([2H])c1[2H]] |
| 1-191 | 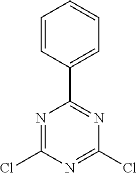 [SMILES: Clc1nc(Cl)nc(-c2ccccc2)n1] | 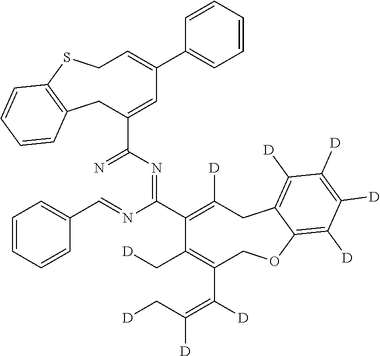 [SMILES: [2H]C\C([2H])=C([2H])/C1=C(C[2H])/C(C(=N/C(=N)/C2=C/C(c3ccccc3)=C\CSc3ccccc3C2)/N=C/c2ccccc2)=C(/[2H])Cc2c([2H])c([2H])c([2H])c([2H])c2OC1] |
| 1-192 | 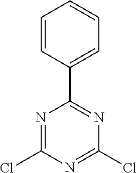 [SMILES: Clc1nc(Cl)nc(-c2ccccc2)n1] | 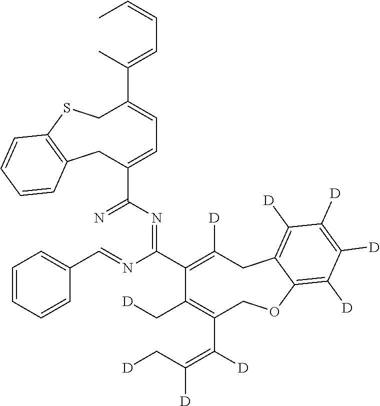 [SMILES: [2H]C\C([2H])=C([2H])/C1=C(C[2H])/C(C(=N/C(=N)/C2=C/C=C(/C(C)=C/C=C\C)CSc3ccccc3C2)/N=C/c2ccccc2)=C(/[2H])Cc2c([2H])c([2H])c([2H])c([2H])c2OC1] |
| 1-201 | 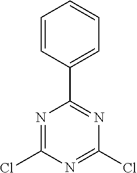 [SMILES: Clc1nc(Cl)nc(-c2ccccc2)n1] | 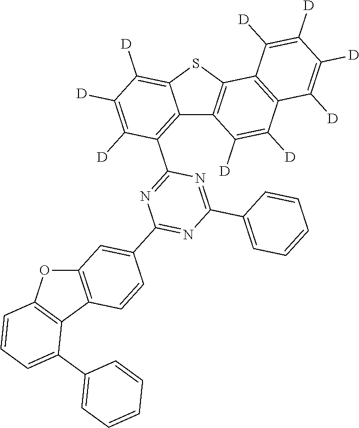 [SMILES: [2H]c1c([2H])c(-c2nc(-c3ccccc3)nc(-c3ccc4c(c3)oc3cccc(-c5ccccc5)c34)n2)c2c(sc3c4c([2H])c([2H])c([2H])c([2H])c4c([2H])c([2H])c32)c1[2H]] |
| 1-202 | 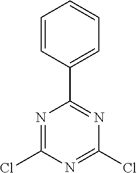 [SMILES: Clc1nc(Cl)nc(-c2ccccc2)n1] | 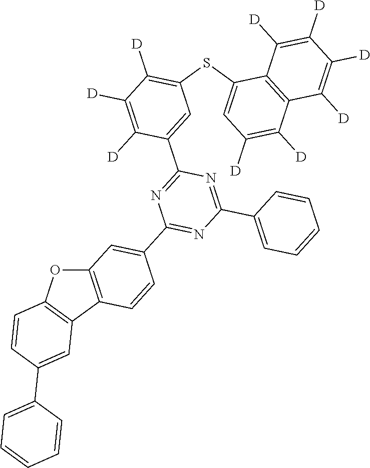 [SMILES: [2H]c1cc(Sc2cc(-c3nc(-c4ccccc4)nc(-c4ccc5c(c4)oc4ccc(-c6ccccc6)cc45)n3)c([2H])c([2H])c2[2H])c2c([2H])c([2H])c([2H])c([2H])c2c1[2H]] |
| 1-203 | 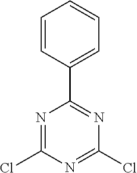 [SMILES: Clc1nc(Cl)nc(-c2ccccc2)n1] | 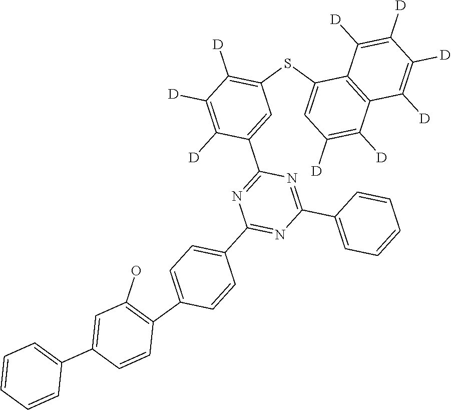 [SMILES: [2H]c1cc(Sc2cc(-c3nc(-c4ccccc4)nc(-c4ccc(-c5ccc(-c6ccccc6)cc5O)cc4)n3)c([2H])c([2H])c2[2H])c2c([2H])c([2H])c([2H])c([2H])c2c1[2H]] |
| 1-204 | 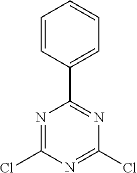 [SMILES: Clc1nc(Cl)nc(-c2ccccc2)n1] | 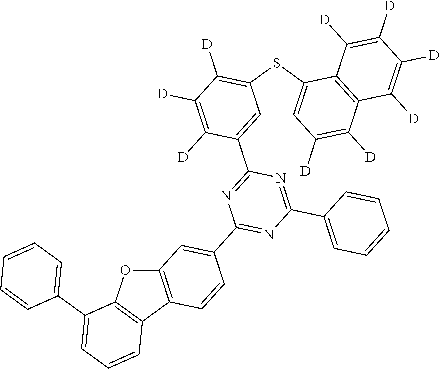 [SMILES: [2H]c1cc(Sc2cc(-c3nc(-c4ccccc4)nc(-c4ccc5c(c4)oc4c(-c6ccccc6)cccc45)n3)c([2H])c([2H])c2[2H])c2c([2H])c([2H])c([2H])c([2H])c2c1[2H]] |
| 1-205 | 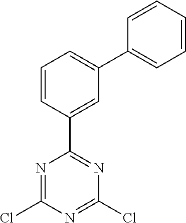 [SMILES: Clc1nc(Cl)nc(-c2cccc(-c3ccccc3)c2)n1] | 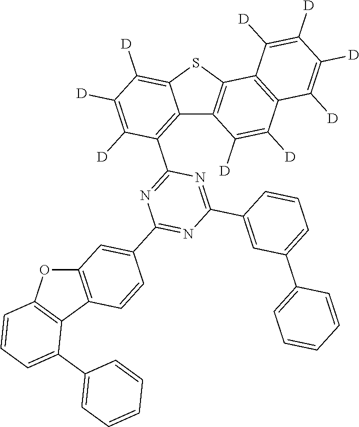 [SMILES: [2H]c1c([2H])c(-c2nc(-c3cccc(-c4ccccc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H]] |
| 1-206 | 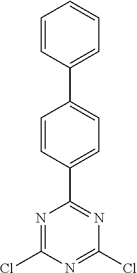 [SMILES: Clc1nc(Cl)nc(-c2ccc(-c3ccccc3)cc2)n1] | 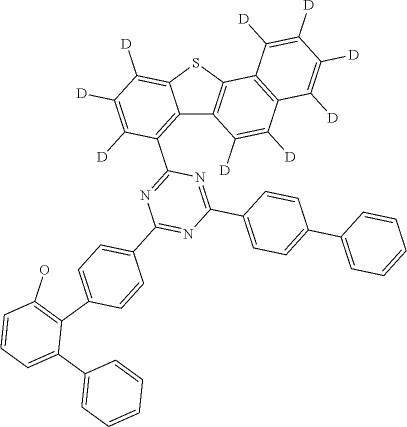 [SMILES: [2H]c1c([2H])c(-c2nc(-c3ccc(-c4ccccc4)cc3)nc(-c3ccc(-c4c(O)cccc4-c4ccccc4)cc3)n2)c2c(sc3c4c([2H])c([2H])c([2H])c([2H])c4c([2H])c([2H])c32)c1[2H]] |
| 1-207 | 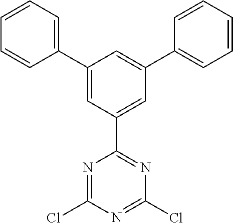 [SMILES: Clc1nc(Cl)nc(-c2cc(-c3ccccc3)cc(-c3ccccc3)c2)n1] | 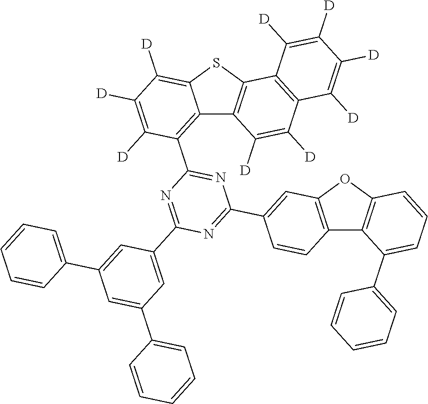 [SMILES: [2H]c1c([2H])c(-c2nc(-c3cc(-c4ccccc4)cc(-c4ccccc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H]] |
| 1-208 | 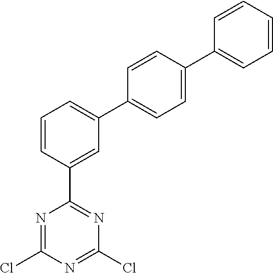 [SMILES: Clc1nc(Cl)nc(-c2cccc(-c3ccc(-c4ccccc4)cc3)c2)n1] | 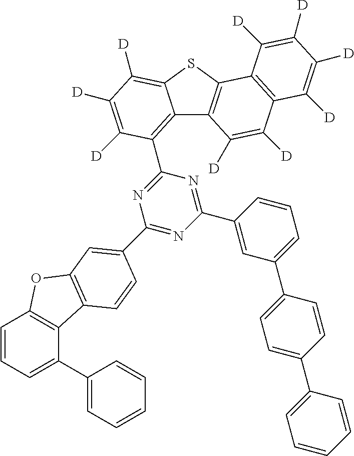 [SMILES: [2H]c1c([2H])c(-c2nc(-c3cccc(-c4ccc(-c5ccccc5)cc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H]] |
| 1-213 | 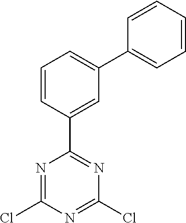 [SMILES: Clc1nc(Cl)nc(-c2cccc(-c3ccccc3)c2)n1] | 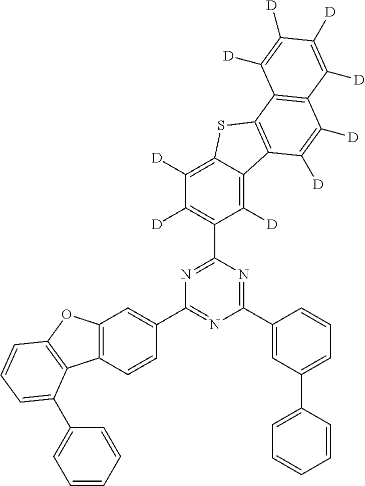 [SMILES: [2H]c1c(-c2nc(-c3cccc(-c4ccccc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H]] |
| 1-214 | 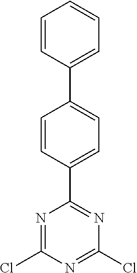 [SMILES: Clc1nc(Cl)nc(-c2ccc(-c3ccccc3)cc2)n1] | 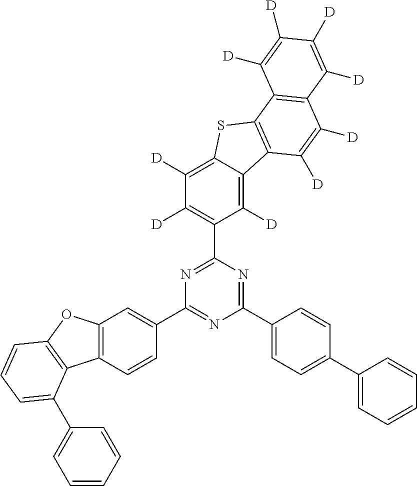 [SMILES: [2H]c1c(-c2nc(-c3ccc(-c4ccccc4)cc3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H]] |
| 1-215 | 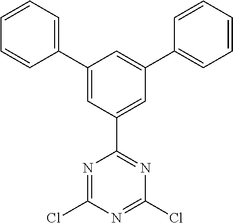 [SMILES: Clc1nc(Cl)nc(-c2cc(-c3ccccc3)cc(-c3ccccc3)c2)n1] | 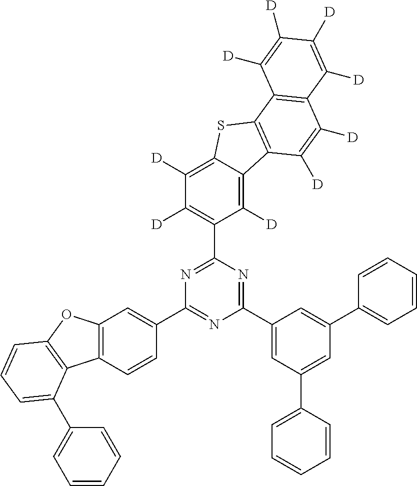 [SMILES: [2H]c1c(-c2nc(-c3cc(-c4ccccc4)cc(-c4ccccc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H]] |
| 1-216 | 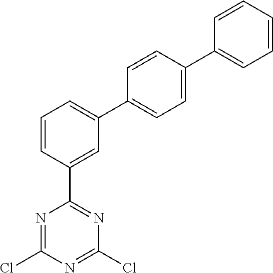 [SMILES: Clc1nc(Cl)nc(-c2cccc(-c3ccc(-c4ccccc4)cc3)c2)n1] | 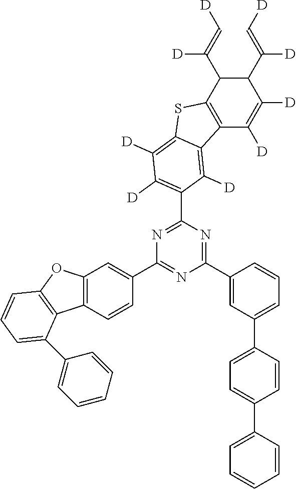 [SMILES: [2H]/C=C(\[2H])C1C([2H])=C([2H])c2c(sc3c([2H])c([2H])c(-c4nc(-c5cccc(-c6ccc(-c7ccccc7)cc6)c5)nc(-c5ccc6c(c5)oc5cccc(-c7ccccc7)c56)n4)c([2H])c23)C1/C([2H])=C/[2H]] |
| 1-217 | 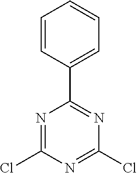 [SMILES: Clc1nc(Cl)nc(-c2ccccc2)n1] | 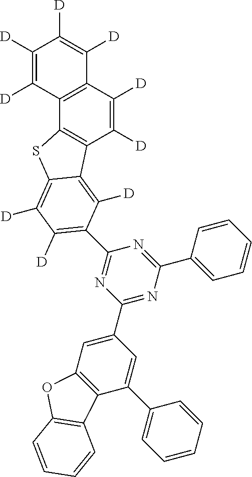 [SMILES: [2H]c1c(-c2nc(-c3ccccc3)nc(-c3cc(-c4ccccc4)c4c(c3)oc3ccccc34)n2)c([2H])c2c(sc3c4c([2H])c([2H])c([2H])c([2H])c4c([2H])c([2H])c32)c1[2H]] |
| 1-218 | 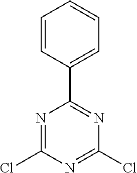 [SMILES: Clc1nc(Cl)nc(-c2ccccc2)n1] | 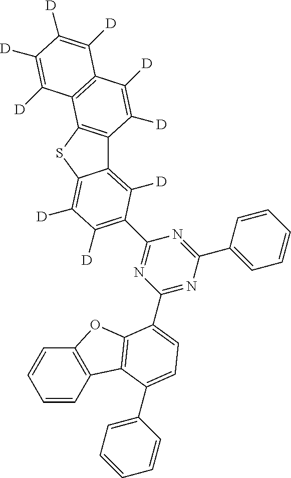 [SMILES: [2H]c1c(-c2nc(-c3ccccc3)nc(-c3ccc(-c4ccccc4)c4c3oc3ccccc34)n2)c([2H])c2c(sc3c4c([2H])c([2H])c([2H])c([2H])c4c([2H])c([2H])c32)c1[2H]] |
| 1-221 | 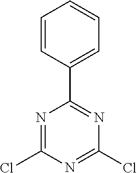 [SMILES: Clc1nc(Cl)nc(-c2ccccc2)n1] | 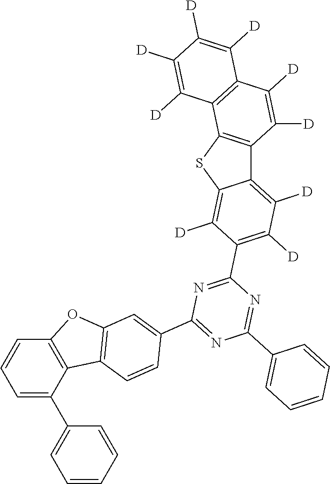 [SMILES: [2H]c1c([2H])c([2H])c2c(c1[2H])c([2H])c([2H])c1c2sc2c([2H])c(-c3nc(-c4ccccc4)nc(-c4ccc5c(c4)oc4cccc(-c6ccccc6)c45)n3)c([2H])c([2H])c21] |
| 1-222 | 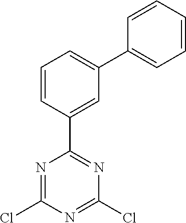 [SMILES: Clc1nc(Cl)nc(-c2cccc(-c3ccccc3)c2)n1] | 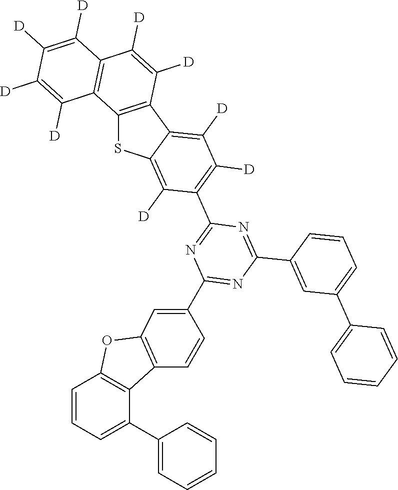 [SMILES: [2H]c1c([2H])c([2H])c2c(c1[2H])c([2H])c([2H])c1c2sc2c([2H])c(-c3nc(-c4cccc(-c5ccccc5)c4)nc(-c4ccc5c(c4)oc4cccc(-c6ccccc6)c45)n3)c([2H])c([2H])c21] |
| 1-223 | 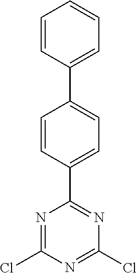 [SMILES: Clc1nc(Cl)nc(-c2ccc(-c3ccccc3)cc2)n1] | 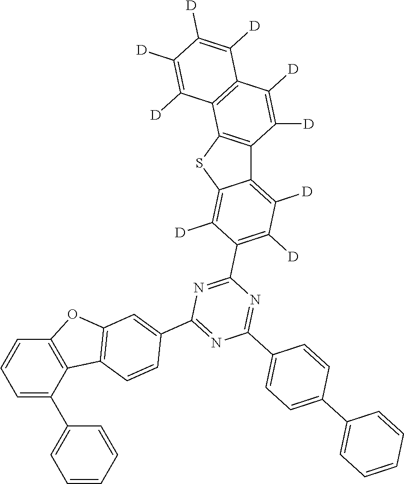 [SMILES: [2H]c1c([2H])c([2H])c2c(c1[2H])c([2H])c([2H])c1c2sc2c([2H])c(-c3nc(-c4ccc(-c5ccccc5)cc4)nc(-c4ccc5c(c4)oc4cccc(-c6ccccc6)c45)n3)c([2H])c([2H])c21] |
| 1-224 | 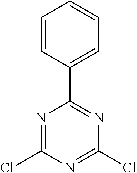 [SMILES: Clc1nc(Cl)nc(-c2ccccc2)n1] | 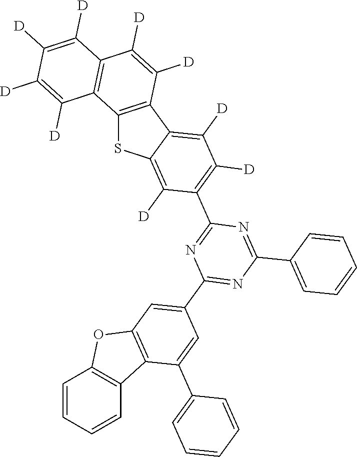 [SMILES: [2H]c1c([2H])c([2H])c2c(c1[2H])c([2H])c([2H])c1c2sc2c([2H])c(-c3nc(-c4ccccc4)nc(-c4cc(-c5ccccc5)c5c(c4)oc4ccccc45)n3)c([2H])c([2H])c21] |
| 1-229 | 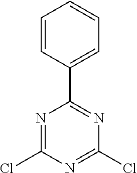 [SMILES: Clc1nc(Cl)nc(-c2ccccc2)n1] | 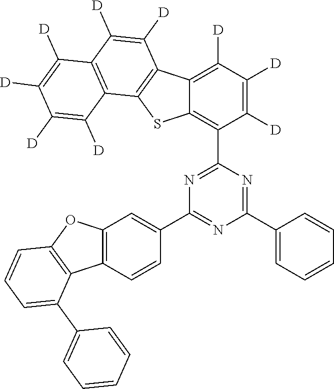 [SMILES: [2H]c1c([2H])c([2H])c2c(sc3c4c([2H])c([2H])c([2H])c([2H])c4c([2H])c([2H])c32)c1-c1nc(-c2ccccc2)nc(-c2ccc3c(c2)oc2cccc(-c4ccccc4)c23)n1] |
| 1-230 | 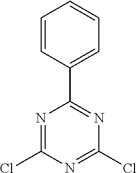 [SMILES: Clc1nc(Cl)nc(-c2ccccc2)n1] | 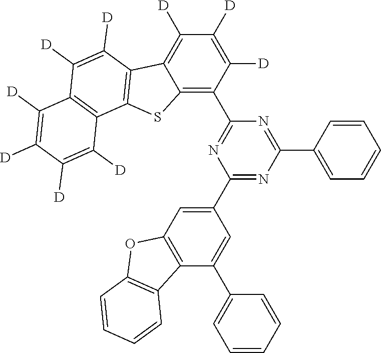 [SMILES: [2H]c1c([2H])c([2H])c2c(sc3c4c([2H])c([2H])c([2H])c([2H])c4c([2H])c([2H])c32)c1-c1nc(-c2ccccc2)nc(-c2cc(-c3ccccc3)c3c(c2)oc2ccccc23)n1] |
| 1-231 | 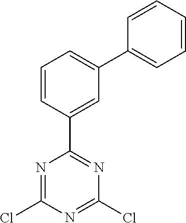 [SMILES: Clc1nc(Cl)nc(-c2cccc(-c3ccccc3)c2)n1] | 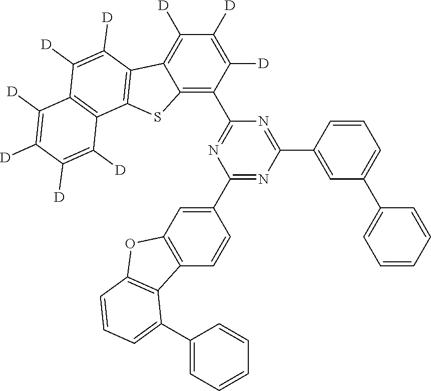 [SMILES: [2H]c1c([2H])c([2H])c2c(sc3c4c([2H])c([2H])c([2H])c([2H])c4c([2H])c([2H])c32)c1-c1nc(-c2cccc(-c3ccccc3)c2)nc(-c2ccc3c(c2)oc2cccc(-c4ccccc4)c23)n1] |
| 1-232 | 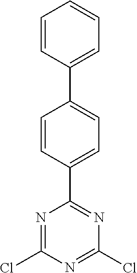 [SMILES: Clc1nc(Cl)nc(-c2ccc(-c3ccccc3)cc2)n1] | 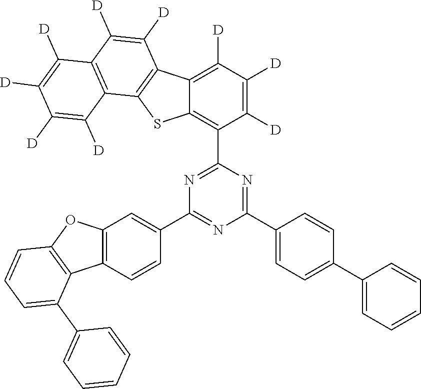 [SMILES: [2H]c1c([2H])c([2H])c2c(sc3c4c([2H])c([2H])c([2H])c([2H])c4c([2H])c([2H])c32)c1-c1nc(-c2ccc(-c3ccccc3)cc2)nc(-c2ccc3c(c2)oc2cccc(-c4ccccc4)c23)n1] |
| 1-237 | 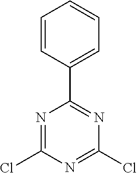 [SMILES: Clc1nc(Cl)nc(-c2ccccc2)n1] | 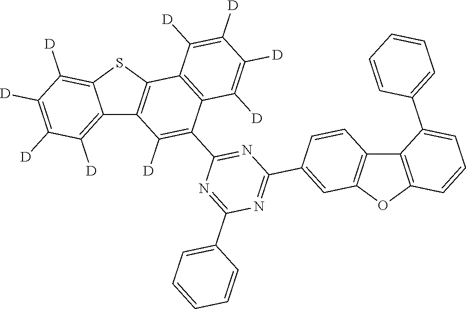 [SMILES: [2H]c1c([2H])c([2H])c2c(sc3c4c([2H])c([2H])c([2H])c([2H])c4c(-c4nc(-c5ccccc5)nc(-c5ccc6c(c5)oc5cccc(-c7ccccc7)c56)n4)c([2H])c32)c1[2H]] |
| 1-238 | 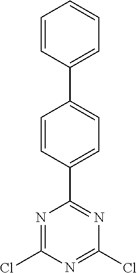 [SMILES: Clc1nc(Cl)nc(-c2ccc(-c3ccccc3)cc2)n1] | 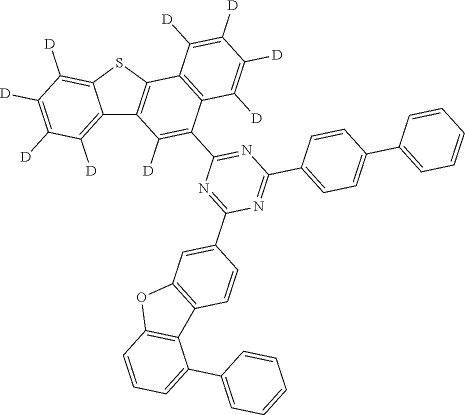 [SMILES: [2H]c1c([2H])c([2H])c2c(sc3c4c([2H])c([2H])c([2H])c([2H])c4c(-c4nc(-c5ccc(-c6ccccc6)cc5)nc(-c5ccc6c(c5)oc5cccc(-c7ccccc7)c56)n4)c([2H])c32)c1[2H]] |
| 1-242 | 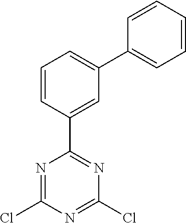 [SMILES: Clc1nc(Cl)nc(-c2cccc(-c3ccccc3)c2)n1] | 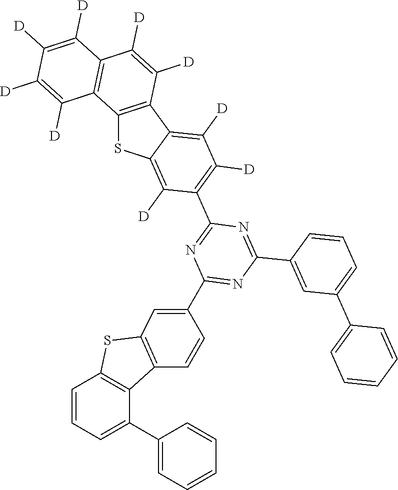 [SMILES: [2H]c1c([2H])c([2H])c2c(c1[2H])c([2H])c([2H])c1c2sc2c([2H])c(-c3nc(-c4cccc(-c5ccccc5)c4)nc(-c4ccc5c(c4)sc4cccc(-c6ccccc6)c45)n3)c([2H])c([2H])c21] |
| 1-243 | 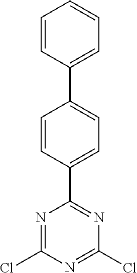 [SMILES: Clc1nc(Cl)nc(-c2ccc(-c3ccccc3)cc2)n1] | 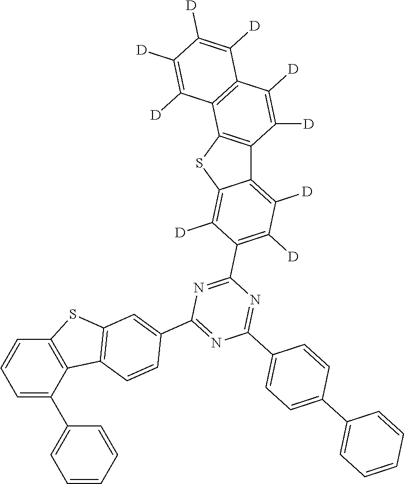 [SMILES: [2H]c1c([2H])c([2H])c2c(c1[2H])c([2H])c([2H])c1c2sc2c([2H])c(-c3nc(-c4ccc(-c5ccccc5)cc4)nc(-c4ccc5c(c4)sc4cccc(-c6ccccc6)c45)n3)c([2H])c([2H])c21] |
| 1-244 | 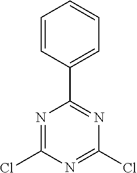 [SMILES: Clc1nc(Cl)nc(-c2ccccc2)n1] | 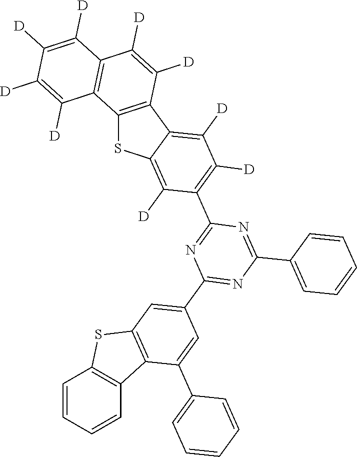 [SMILES: [2H]c1c([2H])c([2H])c2c(c1[2H])c([2H])c([2H])c1c2sc2c([2H])c(-c3nc(-c4ccccc4)nc(-c4cc(-c5ccccc5)c5c(c4)sc4ccccc45)n3)c([2H])c([2H])c21] |
| 1-249 | 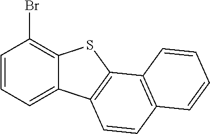 [SMILES: Brc1cccc2c1sc1c3ccccc3ccc21] | 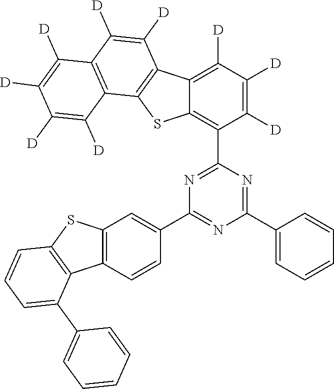 [SMILES: [2H]c1c([2H])c([2H])c2c(sc3c4c([2H])c([2H])c([2H])c([2H])c4c([2H])c([2H])c32)c1-c1nc(-c2ccccc2)nc(-c2ccc3c(c2)sc2cccc(-c4ccccc4)c23)n1] |
| 1-250 | 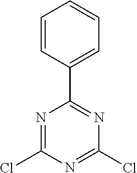 [SMILES: Clc1nc(Cl)nc(-c2ccccc2)n1] | 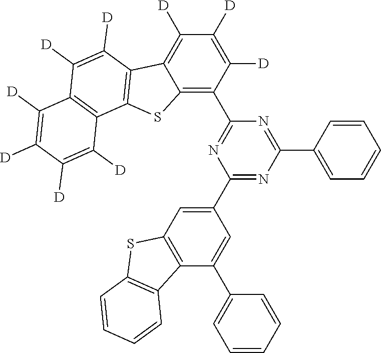 [SMILES: [2H]c1c([2H])c([2H])c2c(sc3c4c([2H])c([2H])c([2H])c([2H])c4c([2H])c([2H])c32)c1-c1nc(-c2ccccc2)nc(-c2cc(-c3ccccc3)c3c(c2)sc2ccccc23)n1] |
| 1-251 | 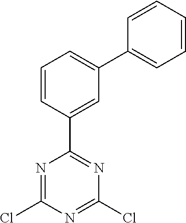 [SMILES: Clc1nc(Cl)nc(-c2cccc(-c3ccccc3)c2)n1] | 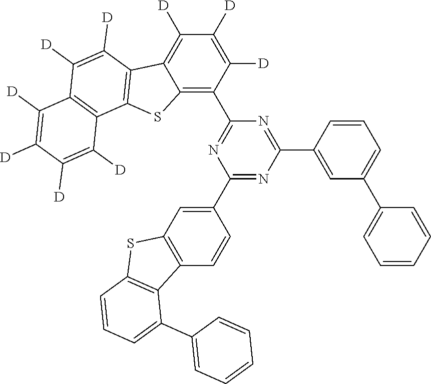 [SMILES: [2H]c1c([2H])c([2H])c2c(sc3c4c([2H])c([2H])c([2H])c([2H])c4c([2H])c([2H])c32)c1-c1nc(-c2cccc(-c3ccccc3)c2)nc(-c2ccc3c(c2)sc2cccc(-c4ccccc4)c23)n1] |
| 1-252 | 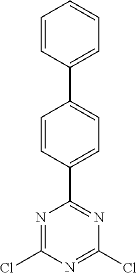 [SMILES: Clc1nc(Cl)nc(-c2ccc(-c3ccccc3)cc2)n1] | 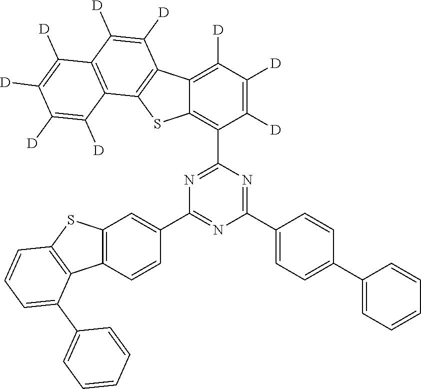 [SMILES: [2H]c1c([2H])c([2H])c2c(sc3c4c([2H])c([2H])c([2H])c([2H])c4c([2H])c([2H])c32)c1-c1nc(-c2ccc(-c3ccccc3)cc2)nc(-c2ccc3c(c2)sc2cccc(-c4ccccc4)c23)n1] |
| 1-257 | 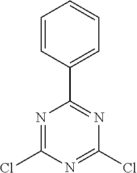 [SMILES: Clc1nc(Cl)nc(-c2ccccc2)n1] | 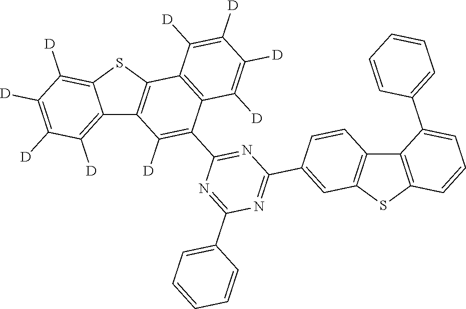 [SMILES: [2H]c1c([2H])c([2H])c2c(sc3c4c([2H])c([2H])c([2H])c([2H])c4c(-c4nc(-c5ccccc5)nc(-c5ccc6c(c5)sc5cccc(-c7ccccc7)c56)n4)c([2H])c32)c1[2H]] |
| 1-258 | 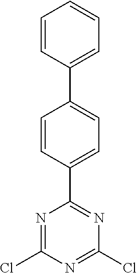 [SMILES: Clc1nc(Cl)nc(-c2ccc(-c3ccccc3)cc2)n1] | 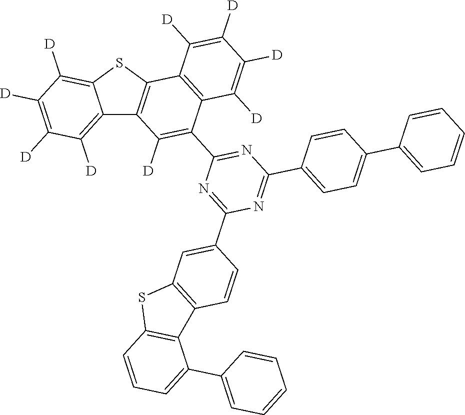 [SMILES: [2H]c1c([2H])c([2H])c2c(sc3c4c([2H])c([2H])c([2H])c([2H])c4c(-c4nc(-c5ccc(-c6ccccc6)cc5)nc(-c5ccc6c(c5)sc5cccc(-c7ccccc7)c56)n4)c([2H])c32)c1[2H]] |

| Com- | |
|---|---|
| pound | Yield |
| 1-2 | 83 |
| 1-3 | 81 |
| 1-4 | 80 |
| 1-5 | 84 |
| 1-6 | 85 |
| 1-7 | 84 |
| 1-8 | 83 |
| 1-9 | 82 |
| 1-10 | 83 |
| 1-11 | 84 |
| 1-12 | 80 |
| 1-21 | 87 |
| 1-22 | 81 |
| 1-23 | 82 |
| 1-24 | 80 |
| 1-25 | 81 |
| 1-26 | 80 |
| 1-27 | 82 |
| 1-28 | 83 |
| 1-29 | 82 |
| 1-30 | 81 |
| 1-31 | 84 |
| 1-32 | 83 |
| 1-41 | 84 |
| 1-42 | 82 |
| 1-43 | 81 |
| 1-44 | 83 |
| 1-45 | 81 |
| 1-46 | 80 |
| 1-47 | 84 |
| 1-48 | 85 |
| 1-49 | 84 |
| 1-50 | 83 |
| 1-51 | 82 |
| 1-52 | 83 |
| 1-61 | 84 |
| 1-62 | 80 |
| 1-63 | 87 |
| 1-64 | 81 |
| 1-65 | 82 |
| 1-66 | 80 |
| 1-67 | 81 |
| 1-68 | 80 |
| 1-69 | 82 |
| 1-70 | 83 |
| 1-71 | 82 |
| 1-72 | 81 |
| 1-81 | 84 |
| 1-82 | 83 |
| 1-83 | 84 |
| 1-84 | 82 |
| 1-85 | 81 |
| 1-86 | 83 |
| 1-87 | 81 |
| 1-88 | 80 |
| 1-89 | 84 |
| 1-90 | 85 |
| 1-91 | 84 |
| 1-92 | 83 |
| 1-101 | 82 |
| 1-102 | 82 |
| 1-103 | 81 |
| 1-104 | 84 |
| 1-105 | 83 |
| 1-106 | 84 |
| 1-107 | 82 |
| 1-108 | 81 |
| 1-109 | 83 |
| 1-110 | 81 |
| 1-111 | 80 |
| 1-112 | 82 |
| 1-121 | 81 |
| 1-122 | 84 |
| 1-123 | 83 |
| 1-124 | 84 |
| 1-125 | 82 |
| 1-126 | 81 |
| 1-127 | 83 |
| 1-128 | 81 |
| 1-129 | 80 |
| 1-130 | 81 |
| 1-131 | 83 |
| 1-132 | 81 |
| 1-141 | 80 |
| 1-142 | 82 |
| 1-143 | 81 |
| 1-144 | 84 |
| 1-145 | 83 |
| 1-146 | 84 |
| 1-147 | 82 |
| 1-148 | 81 |
| 1-149 | 83 |
| 1-150 | 81 |
| 1-151 | 80 |
| 1-152 | 81 |
| 1-161 | 83 |
| 1-162 | 81 |
| 1-163 | 80 |
| 1-164 | 82 |
| 1-165 | 81 |
| 1-166 | 84 |
| 1-167 | 83 |
| 1-168 | 84 |
| 1-169 | 82 |
| 1-170 | 81 |
| 1-171 | 81 |
| 1-172 | 83 |
| 1-181 | 81 |
| 1-182 | 81 |
| 1-183 | 80 |
| 1-184 | 82 |
| 1-185 | 81 |
| 1-186 | 84 |
| 1-187 | 83 |
| 1-188 | 84 |
| 1-189 | 82 |
| 1-190 | 81 |
| 1-191 | 81 |
| 1-192 | 83 |
| 1-201 | 81 |
| 1-202 | 81 |
| 1-203 | 80 |
| 1-204 | 82 |
| 1-205 | 81 |
| 1-206 | 84 |
| 1-207 | 83 |
| 1-208 | 84 |
| 1-213 | 82 |
| 1-214 | 81 |
| 1-215 | 81 |
| 1-216 | 83 |
| 1-217 | 81 |
| 1-218 | 80 |
| 1-221 | 82 |
| 1-222 | 81 |
| 1-223 | 84 |
| 1-224 | 83 |
| 1-229 | 84 |
| 1-230 | 82 |
| 1-231 | 81 |
| 1-232 | 83 |
| 1-237 | 81 |
| 1-238 | 80 |
| 1-242 | 80 |
| 1-243 | 82 |
| 1-244 | 81 |
| 1-249 | 84 |
| 1-250 | 83 |
| 1-251 | 84 |
| 1-252 | 82 |
| 1-257 | 81 |
| 1-258 | 83 |

### [Preparation Example 2] Preparation of Compound 1-13

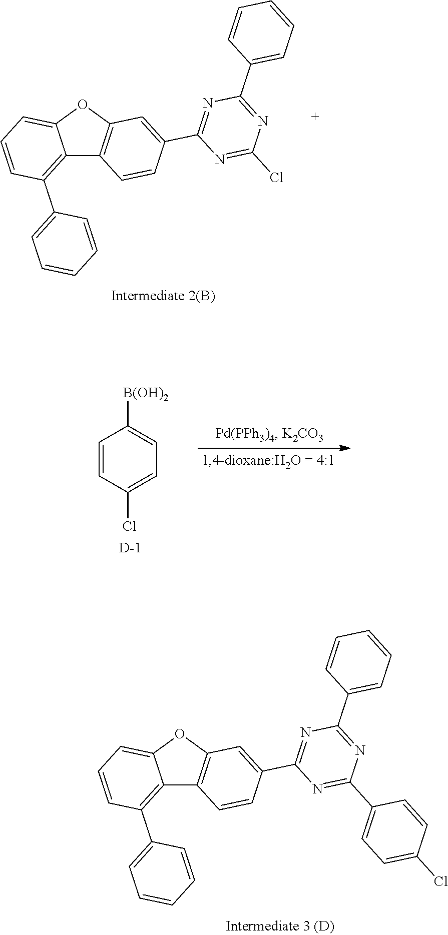

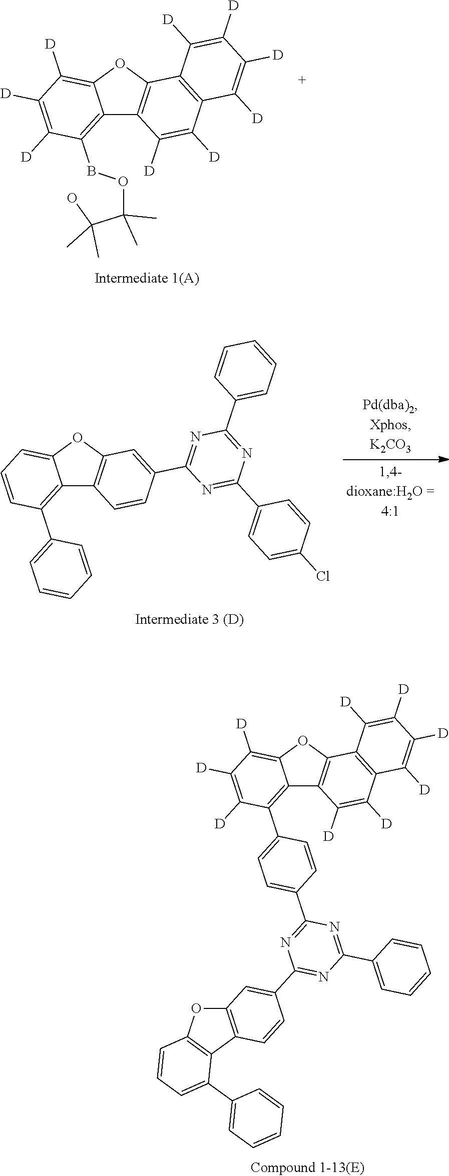

### Synthesis of Intermediate 3(D)

Intermediate 2(B) (10 g, 23 mmol), (4-chlorophenyl)boronic acid (D-1) (7.2 g, 46 mmol), tetrakis(triphenylphosphine)palladium(0) (1.3 g, 1.2 mmol), potassium carbonate (9.5 g, 69 mmol), a 1,4-dioxane/water mixture (100 mL/25 mL) were put into a reaction flask and refluxed at 120° C. for 3 hours. After completing the reaction, a solid produced after lowering the temperature to room temperature was washed with distilled water and MeOH, thereby obtaining Intermediate 3(D) (9 g, 77%).

### Synthesis of Compound 1-13(E)

Intermediate 1(A) (10 g, 53.28 mmol), Intermediate 3(D) (17.3 g, 34 mmol), Pd(dba)₂ (0.8 g, 1.4 mmol), potassium carbonate (11.7 g, 84.9 mmol), Xphos (1.3 g, 2.8 mmol), and a 1,4-dioxane/water mixture (100 mL/25 mL) were put into a reaction flask and refluxed at 120° C. for 3 hours. After completing the reaction, a solid produced after lowering the temperature to room temperature was washed with distilled water and MeOH, thereby obtaining desired Compound 1-13 (E) (13 g, 65%).

In Preparation Example 2, Intermediate 1(A) and Intermediate 2(B) were synthesized in the same manner as in Preparation Example 1 described above.

In Preparation Example 2, desired Compound E below was synthesized in the same manner as above, except that a different type of Reagent D-1 in Table 2 below was used.

[Table 2]

TABLE 2

| Compound | D-1 | Compound C | Yield (%) |
|---|---|---|---|
| 1-13 | 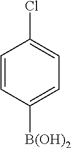 | 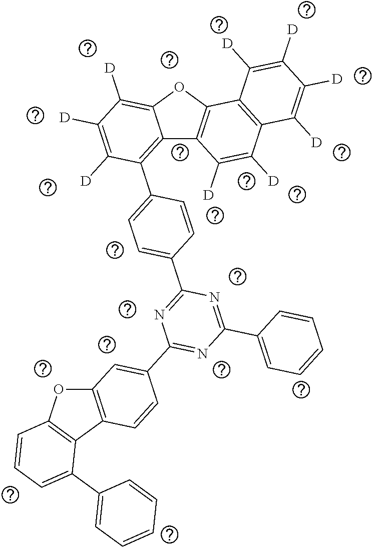 [SMILES: [2H]c1c([2H])c(-c2ccc(-c3nc(-c4ccccc4)nc(-c4ccc5c(c4)oc4cccc(-c6ccccc6)c45)n3)cc2)c2c(oc3c4c([2H])c([2H])c([2H])c([2H])c4c([2H])c([2H])c32)c1[2H]] | 73 |
| 1-14 | 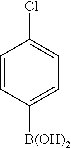 | 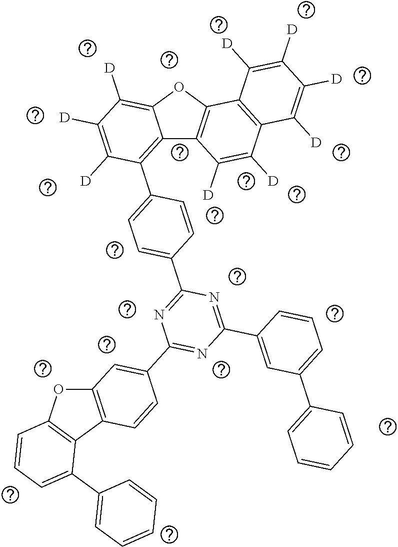 [SMILES: [2H]c1c([2H])c(-c2ccc(-c3nc(-c4cccc(-c5ccccc5)c4)nc(-c4ccc5c(c4)oc4cccc(-c6ccccc6)c45)n3)cc2)c2c(oc3c4c([2H])c([2H])c([2H])c([2H])c4c([2H])c([2H])c32)c1[2H]] | 78 |
| 1-15 | 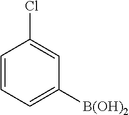 | 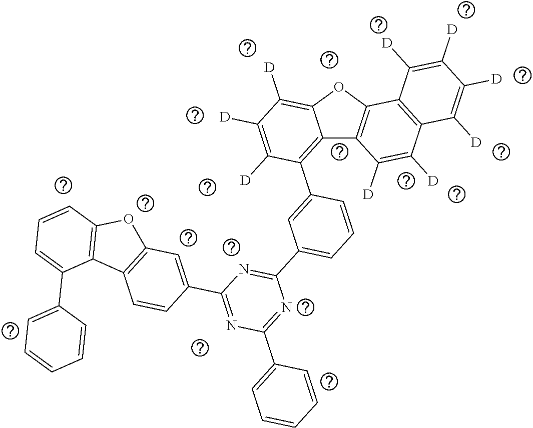 [SMILES: [2H]c1c([2H])c(-c2cccc(-c3nc(-c4ccccc4)nc(-c4ccc5c(c4)oc4cccc(-c6ccccc6)c45)n3)c2)c2c(oc3c4c([2H])c([2H])c([2H])c([2H])c4c([2H])c([2H])c32)c1[2H]] | 76 |
| 1-16 | 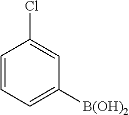 | 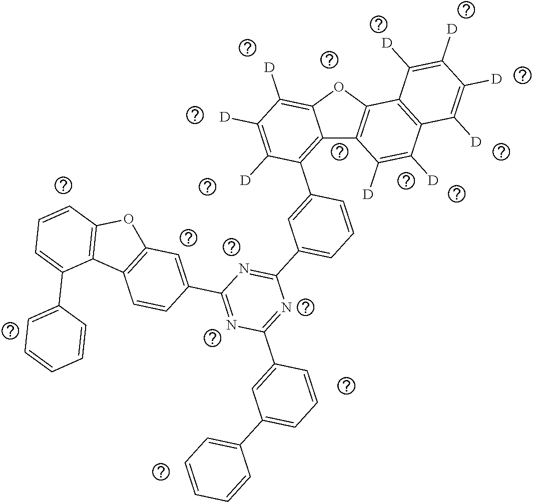 [SMILES: [2H]c1c([2H])c(-c2cccc(-c3nc(-c4cccc(-c5ccccc5)c4)nc(-c4ccc5c(c4)oc4cccc(-c6ccccc6)c45)n3)c2)c2c(oc3c4c([2H])c([2H])c([2H])c([2H])c4c([2H])c([2H])c32)c1[2H]] | 77 |
| 1-17 | 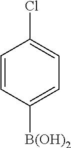 | 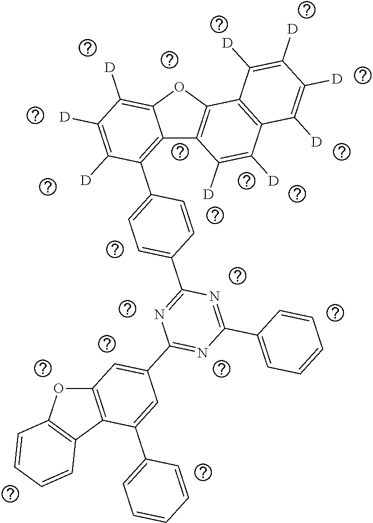 [SMILES: [2H]c1c([2H])c(-c2ccc(-c3nc(-c4ccccc4)nc(-c4cc(-c5ccccc5)c5c(c4)oc4ccccc45)n3)cc2)c2c(oc3c4c([2H])c([2H])c([2H])c([2H])c4c([2H])c([2H])c32)c1[2H]] | 73 |
| 1-18 | 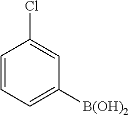 | 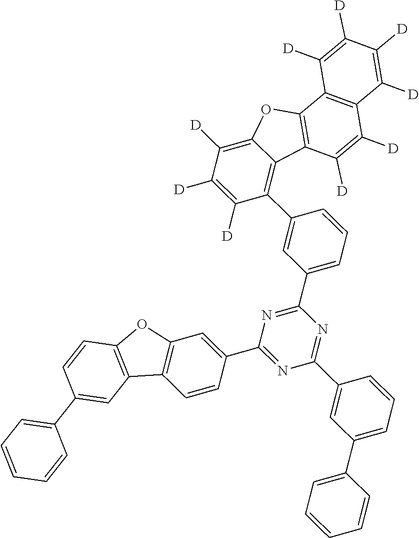 [SMILES: [2H]c1c([2H])c(-c2cccc(-c3nc(-c4cccc(-c5ccccc5)c4)nc(-c4ccc5c(c4)oc4ccc(-c6ccccc6)cc45)n3)c2)c2c(oc3c4c([2H])c([2H])c([2H])c([2H])c4c([2H])c([2H])c32)c1[2H]] | 72 |
| 1-19 | 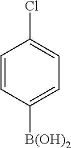 | 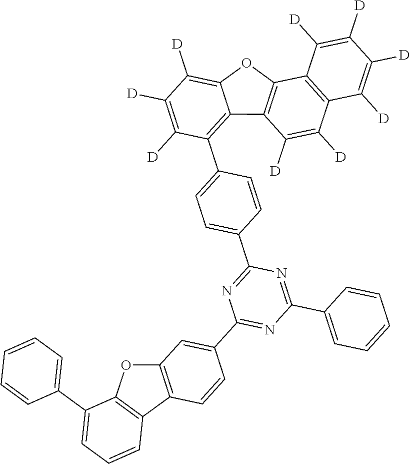 [SMILES: [2H]c1c([2H])c(-c2ccc(-c3nc(-c4ccccc4)nc(-c4ccc5c(c4)oc4c(-c6ccccc6)cccc45)n3)cc2)c2c(oc3c4c([2H])c([2H])c([2H])c([2H])c4c([2H])c([2H])c32)c1[2H]] | 78 |
| 1-20 | 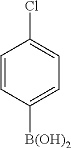 | 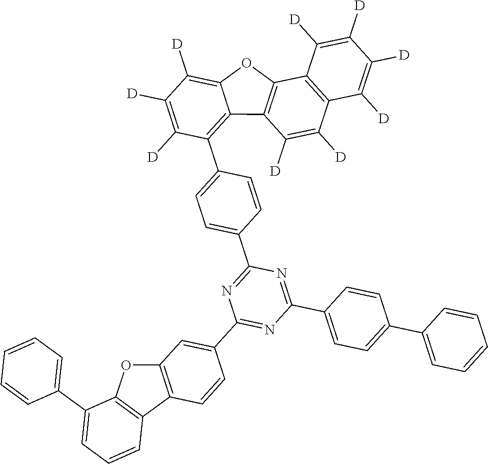 [SMILES: [2H]c1c([2H])c(-c2ccc(-c3nc(-c4ccc(-c5ccccc5)cc4)nc(-c4ccc5c(c4)oc4c(-c6ccccc6)cccc45)n3)cc2)c2c(oc3c4c([2H])c([2H])c([2H])c([2H])c4c([2H])c([2H])c32)c1[2H]] | 80 |
| 1-33 | 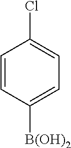 | 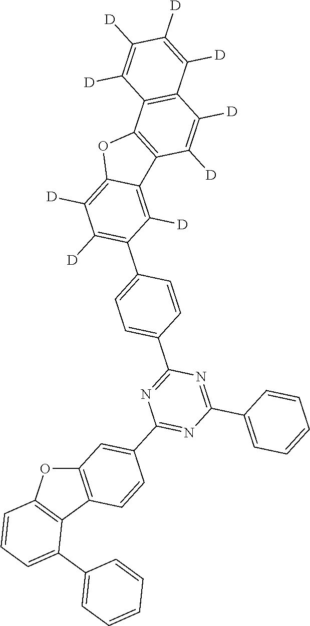 [SMILES: [2H]c1c(-c2ccc(-c3nc(-c4ccccc4)nc(-c4ccc5c(c4)oc4cccc(-c6ccccc6)c45)n3)cc2)c([2H])c2c(oc3c4c([2H])c([2H])c([2H])c([2H])c4c([2H])c([2H])c32)c1[2H]] | 81 |
| 1-34 | 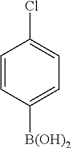 | 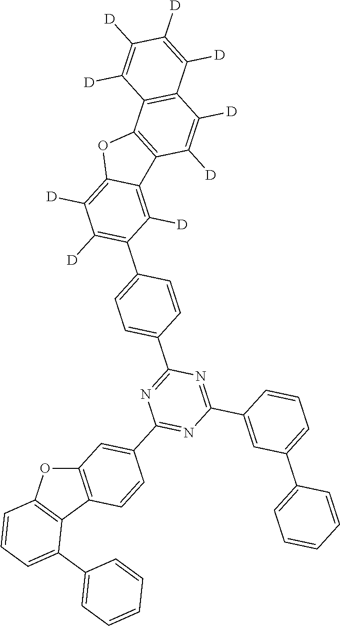 [SMILES: [2H]c1c(-c2ccc(-c3nc(-c4cccc(-c5ccccc5)c4)nc(-c4ccc5c(c4)oc4cccc(-c6ccccc6)c45)n3)cc2)c([2H])c2c(oc3c4c([2H])c([2H])c([2H])c([2H])c4c([2H])c([2H])c32)c1[2H]] | 82 |
| 1-35 | 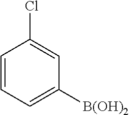 | 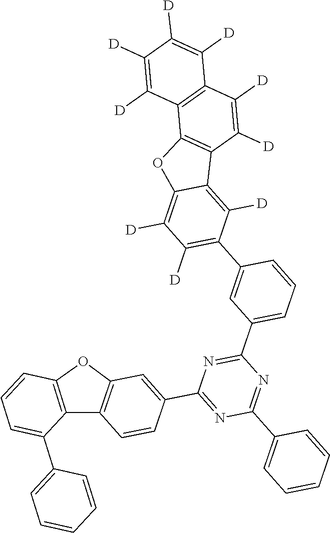 [SMILES: [2H]c1c(-c2cccc(-c3nc(-c4ccccc4)nc(-c4ccc5c(c4)oc4cccc(-c6ccccc6)c45)n3)c2)c([2H])c2c(oc3c4c([2H])c([2H])c([2H])c([2H])c4c([2H])c([2H])c32)c1[2H]] | 78 |
| 1-36 | 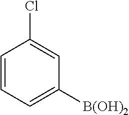 | 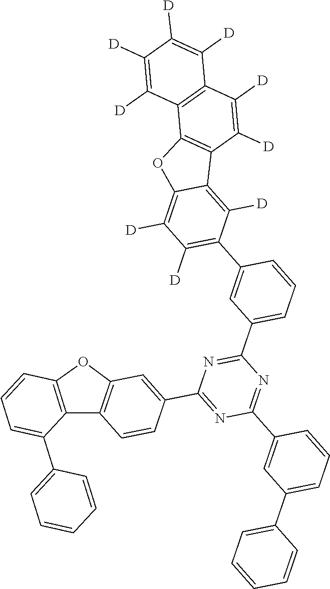 [SMILES: [2H]c1c(-c2cccc(-c3nc(-c4cccc(-c5ccccc5)c4)nc(-c4ccc5c(c4)oc4cccc(-c6ccccc6)c45)n3)c2)c([2H])c2c(oc3c4c([2H])c([2H])c([2H])c([2H])c4c([2H])c([2H])c32)c1[2H]] | 77 |
| 1-37 | 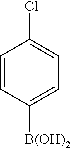 | 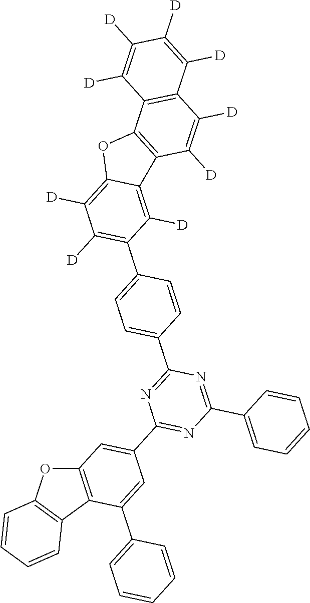 [SMILES: [2H]c1c(-c2ccc(-c3nc(-c4ccccc4)nc(-c4cc(-c5ccccc5)c5c(c4)oc4ccccc45)n3)cc2)c([2H])c2c(oc3c4c([2H])c([2H])c([2H])c([2H])c4c([2H])c([2H])c32)c1[2H]] | 84 |
| 1-38 | 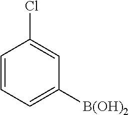 | 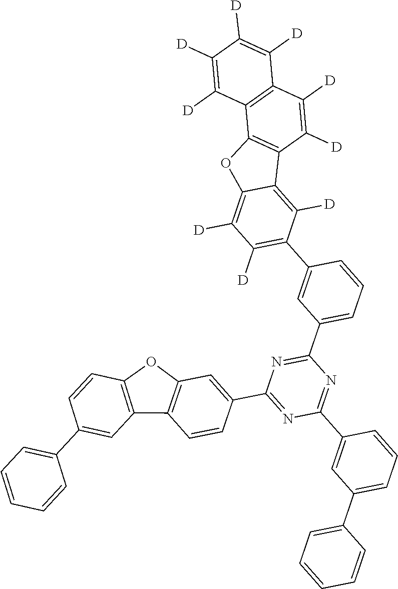 [SMILES: [2H]c1c(-c2cccc(-c3nc(-c4cccc(-c5ccccc5)c4)nc(-c4ccc5c(c4)oc4ccc(-c6ccccc6)cc45)n3)c2)c([2H])c2c(oc3c4c([2H])c([2H])c([2H])c([2H])c4c([2H])c([2H])c32)c1[2H]] | 86 |
| 1-39 | 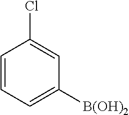 | 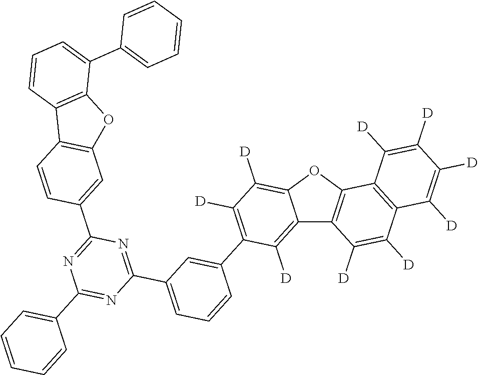 [SMILES: [2H]c1c(-c2cccc(-c3nc(-c4ccccc4)nc(-c4ccc5c(c4)oc4c(-c6ccccc6)cccc45)n3)c2)c([2H])c2c(oc3c4c([2H])c([2H])c([2H])c([2H])c4c([2H])c([2H])c32)c1[2H]] | 87 |
| 1-40 | 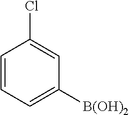 | 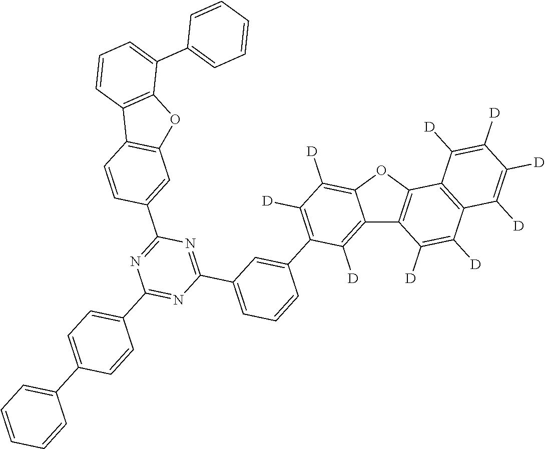 [SMILES: [2H]c1c(-c2cccc(-c3nc(-c4ccc(-c5ccccc5)cc4)nc(-c4ccc5c(c4)oc4c(-c6ccccc6)cccc45)n3)c2)c([2H])c2c(oc3c4c([2H])c([2H])c([2H])c([2H])c4c([2H])c([2H])c32)c1[2H]] | 82 |
| 1-53 | 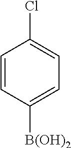 | 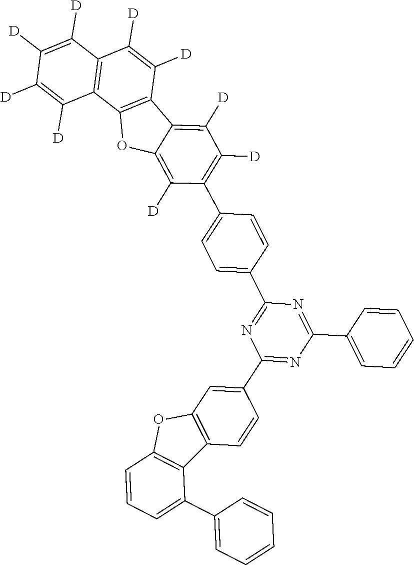 [SMILES: [2H]c1c([2H])c([2H])c2c(c1[2H])c([2H])c([2H])c1c2oc2c([2H])c(-c3ccc(-c4nc(-c5ccccc5)nc(-c5ccc6c(c5)oc5cccc(-c7ccccc7)c56)n4)cc3)c([2H])c([2H])c21] | 79 |
| 1-54 | 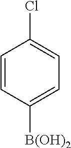 | 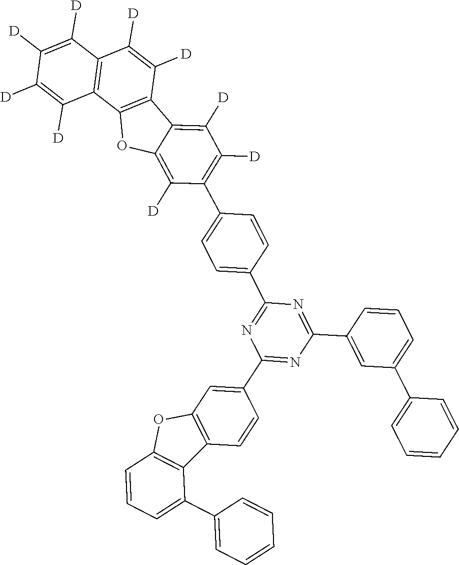 [SMILES: [2H]c1c([2H])c([2H])c2c(c1[2H])c([2H])c([2H])c1c2oc2c([2H])c(-c3ccc(-c4nc(-c5cccc(-c6ccccc6)c5)nc(-c5ccc6c(c5)oc5cccc(-c7ccccc7)c56)n4)cc3)c([2H])c([2H])c21] | 75 |
| 1-55 | 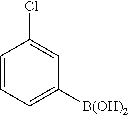 | 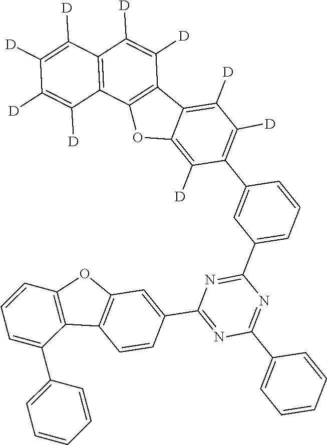 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)oc5cccc(-c7ccccc7)c56)n4)c3)c([2H])c([2H])c21] | 77 |
| 1-56 | 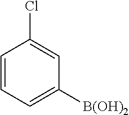 | 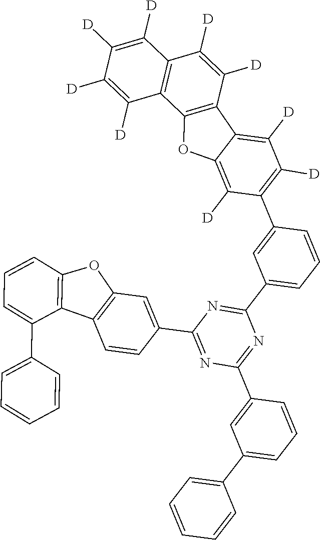 [SMILES: [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)oc5cccc(-c7ccccc7)c56)n4)c3)c([2H])c([2H])c21] | 75 |
| 1-57 | 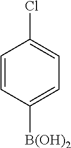 | 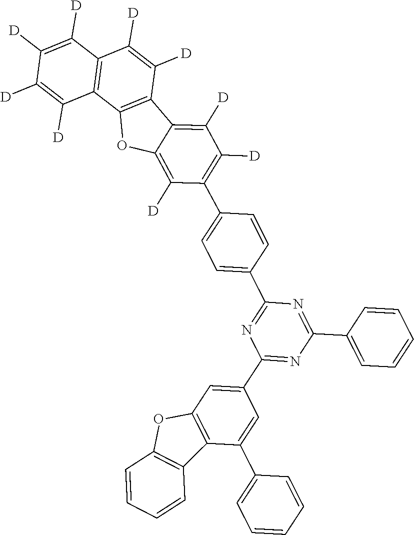 [SMILES: [2H]c1c([2H])c([2H])c2c(c1[2H])c([2H])c([2H])c1c2oc2c([2H])c(-c3ccc(-c4nc(-c5ccccc5)nc(-c5cc(-c6ccccc6)c6c(c5)oc5ccccc56)n4)cc3)c([2H])c([2H])c21] | 84 |
| 1-58 | 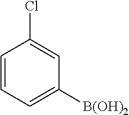 | 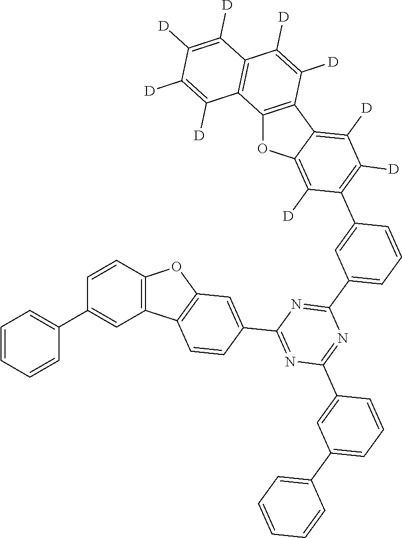 | 82 |
| 1-59 | 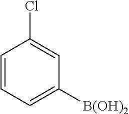 | 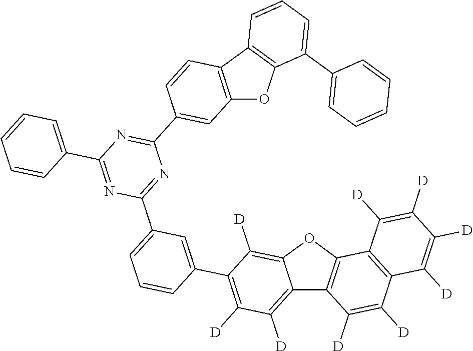 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)oc5c(-c7ccccc7)cccc56)n4)c3)c([2H])c([2H])c21] | 75 |
| 1-60 | 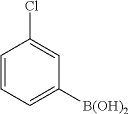 | 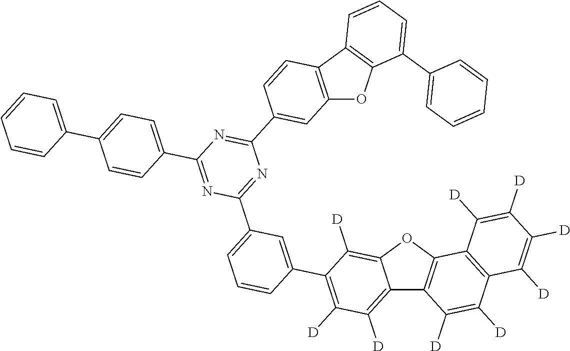 [SMILES: [2H]c1c([2H])c([2H])c2c(c1[2H])c([2H])c([2H])c1c2oc2c([2H])c(-c3cccc(-c4nc(-c5ccc(-c6ccccc6)cc5)nc(-c5ccc6c(c5)oc5c(-c7ccccc7)cccc56)n4)c3)c([2H])c([2H])c21] | 77 |
| 1-73 | 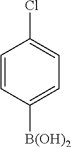 | 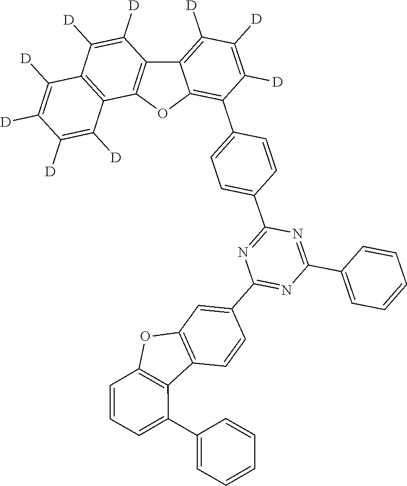 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3ccc4c(c3)oc3cccc(-c5ccccc5)c34)n2)cc1] | 76 |
| 1-74 | 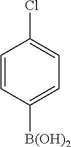 | 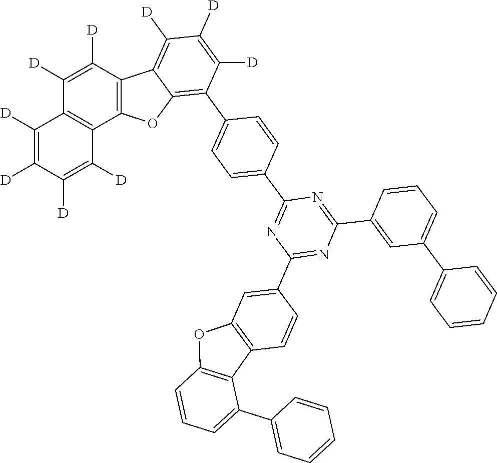 [SMILES: [2H]c1c([2H])c([2H])c2c(oc3c4c([2H])c([2H])c([2H])c([2H])c4c([2H])c([2H])c32)c1-c1ccc(-c2nc(-c3cccc(-c4ccccc4)c3)nc(-c3ccc4c(c3)oc3cccc(-c5ccccc5)c34)n2)cc1] | 79 |
| 1-75 | 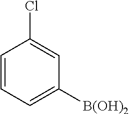 | 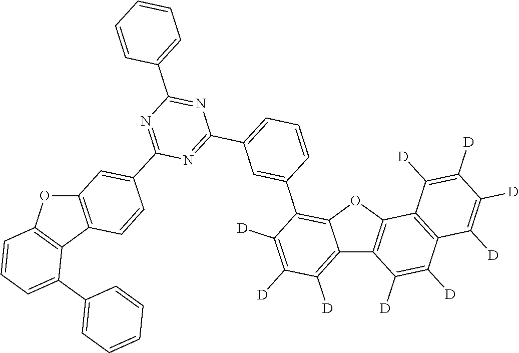 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)oc3cccc(-c5ccccc5)c34)n2)c1] | 80 |
| 1-76 | 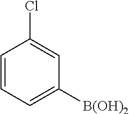 | 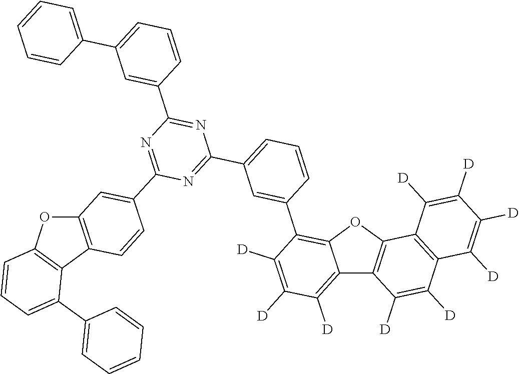 [SMILES: [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3cccc(-c5ccccc5)c34)n2)c1] | 81 |
| 1-77 | 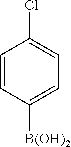 | 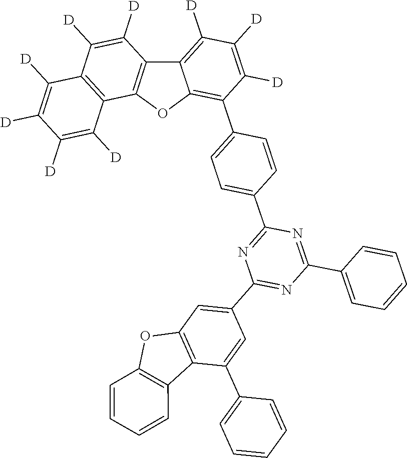 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3cc(-c4ccccc4)c4c(c3)oc3ccccc34)n2)cc1] | 82 |
| 1-78 | 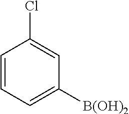 | 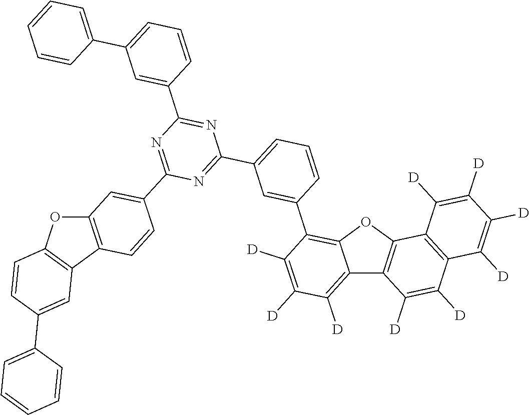 [SMILES: [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3ccc(-c5ccccc5)cc34)n2)c1] | 77 |
| 1-79 | 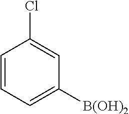 | 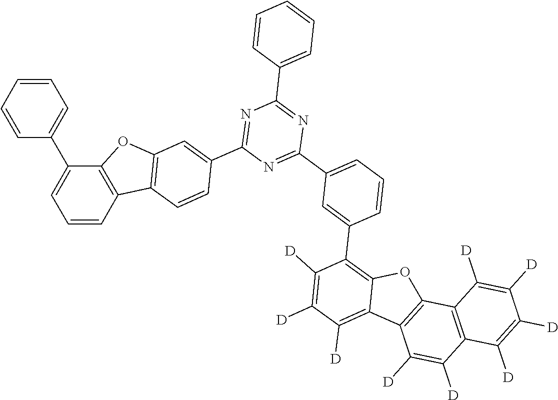 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)oc3c(-c5ccccc5)cccc34)n2)c1] | 70 |
| 1-80 | 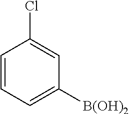 | 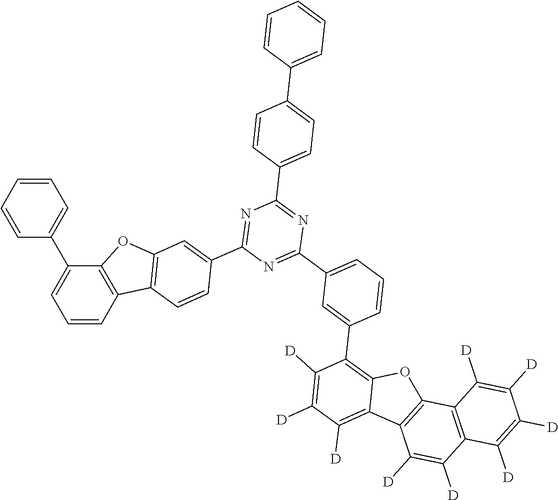 [SMILES: [2H]c1c([2H])c([2H])c2c(oc3c4c([2H])c([2H])c([2H])c([2H])c4c([2H])c([2H])c32)c1-c1cccc(-c2nc(-c3ccc(-c4ccccc4)cc3)nc(-c3ccc4c(c3)oc3c(-c5ccccc5)cccc34)n2)c1] | 79 |
| 1-93 | 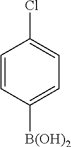 | 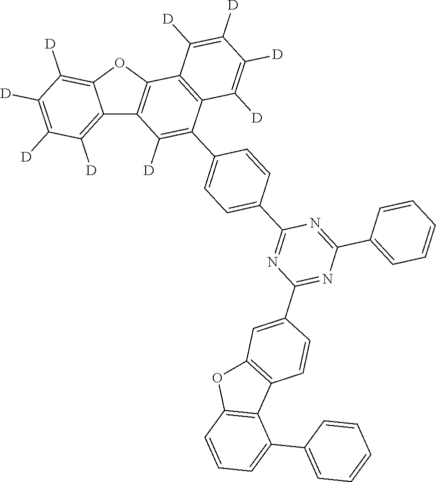 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H]] | 81 |
| 1-94 | 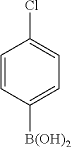 | 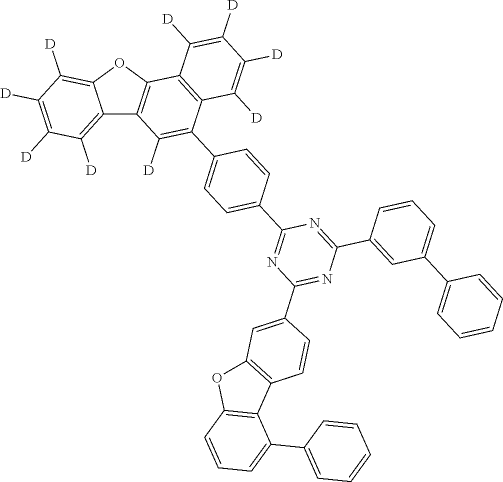 [SMILES: [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]] | 82 |
| 1-95 | 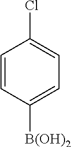 | 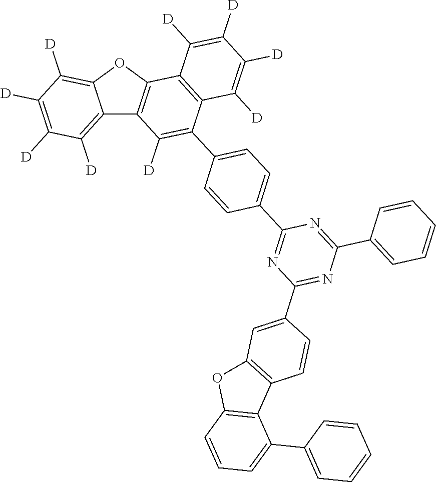 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H]] | 77 |
| 1-96 | 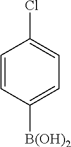 | 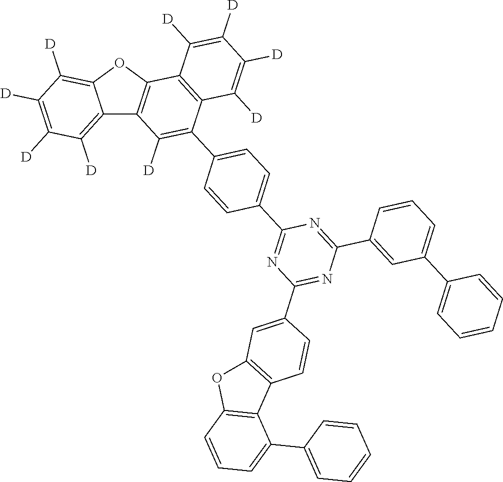 [SMILES: [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]] | 81 |
| 1-97 | 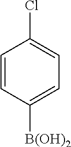 | 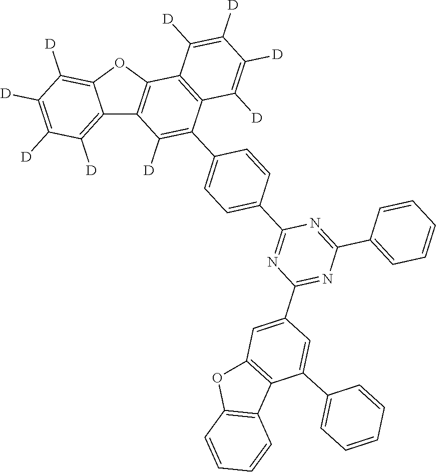 [SMILES: [2H]c1c([2H])c([2H])c2c(oc3c4c([2H])c([2H])c([2H])c([2H])c4c(-c4ccc(-c5nc(-c6ccccc6)nc(-c6cc(-c7ccccc7)c7c(c6)oc6ccccc67)n5)cc4)c([2H])c32)c1[2H]] | 82 |
| 1-98 | 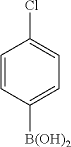 | 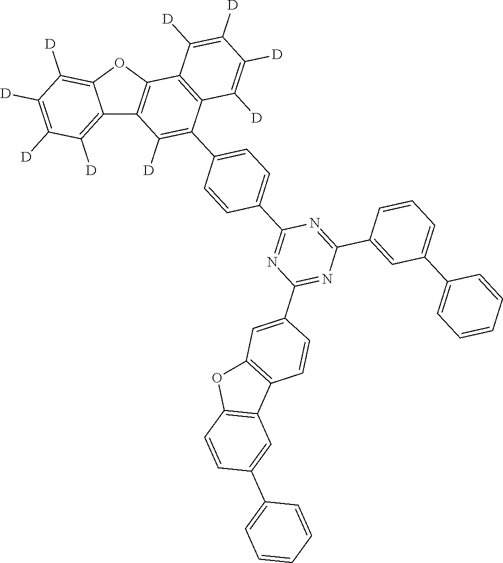 [SMILES: [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6ccc(-c8ccccc8)cc67)n5)cc4)c([2H])c32)c1[2H]] | 77 |
| 1-99 | 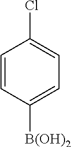 | 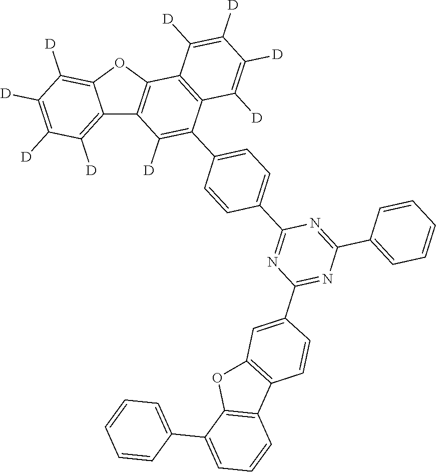 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6c(-c8ccccc8)cccc67)n5)cc4)c([2H])c32)c1[2H]] | 72 |
| 1-100 | 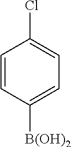 | 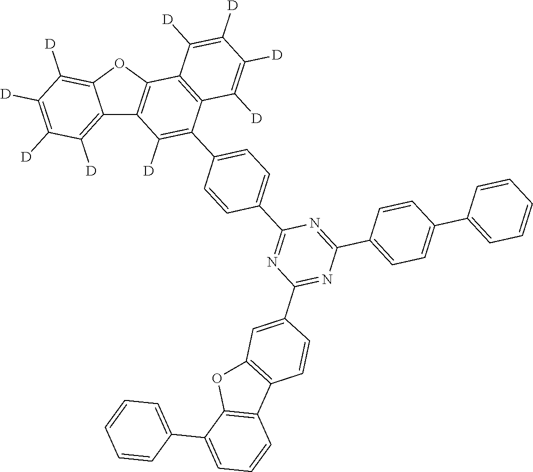 [SMILES: [2H]c1c([2H])c([2H])c2c(oc3c4c([2H])c([2H])c([2H])c([2H])c4c(-c4ccc(-c5nc(-c6ccc(-c7ccccc7)cc6)nc(-c6ccc7c(c6)oc6c(-c8ccccc8)cccc67)n5)cc4)c([2H])c32)c1[2H]] | 79 |
| 1-113 | 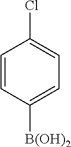 | 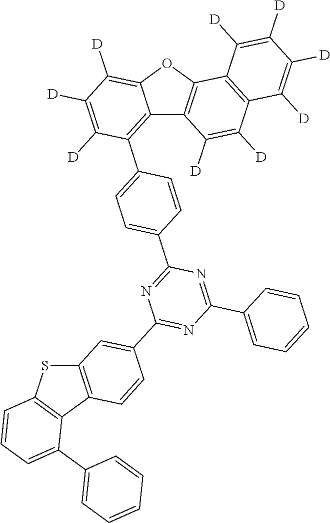 [SMILES: [2H]c1c([2H])c(-c2ccc(-c3nc(-c4ccccc4)nc(-c4ccc5c(c4)sc4cccc(-c6ccccc6)c45)n3)cc2)c2c(oc3c4c([2H])c([2H])c([2H])c([2H])c4c([2H])c([2H])c32)c1[2H]] | 75 |
| 1-114 | 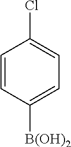 | 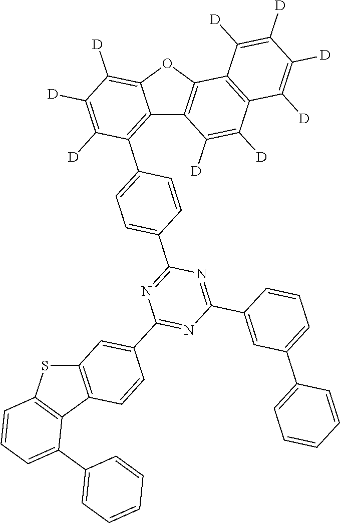 [SMILES: [2H]c1c([2H])c(-c2ccc(-c3nc(-c4cccc(-c5ccccc5)c4)nc(-c4ccc5c(c4)sc4cccc(-c6ccccc6)c45)n3)cc2)c2c(oc3c4c([2H])c([2H])c([2H])c([2H])c4c([2H])c([2H])c32)c1[2H]] | 84 |
| 1-115 | 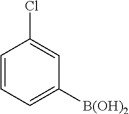 | 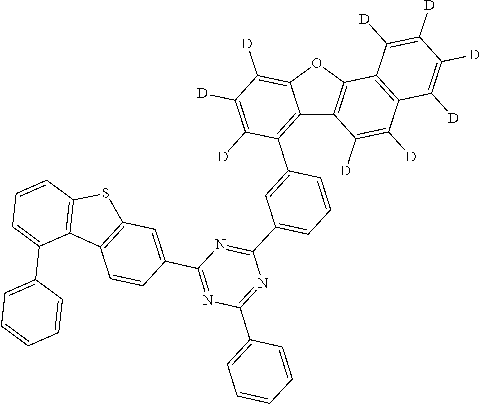 [SMILES: [2H]c1c([2H])c(-c2cccc(-c3nc(-c4ccccc4)nc(-c4ccc5c(c4)sc4cccc(-c6ccccc6)c45)n3)c2)c2c(oc3c4c([2H])c([2H])c([2H])c([2H])c4c([2H])c([2H])c32)c1[2H]] | 85 |
| 1-116 | 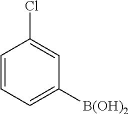 | 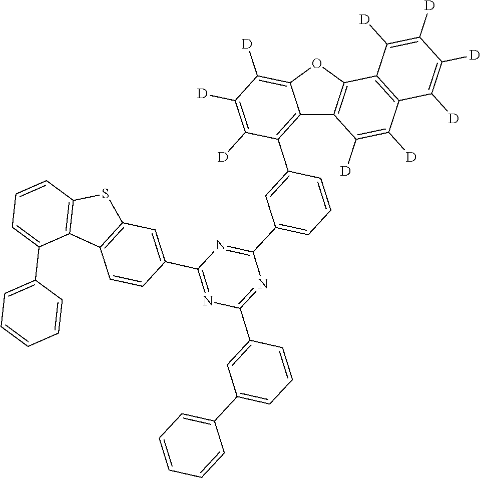 [SMILES: [2H]c1c([2H])c(-c2cccc(-c3nc(-c4cccc(-c5ccccc5)c4)nc(-c4ccc5c(c4)sc4cccc(-c6ccccc6)c45)n3)c2)c2c(oc3c4c([2H])c([2H])c([2H])c([2H])c4c([2H])c([2H])c32)c1[2H]] | 74 |
| 1-117 | 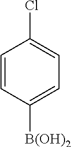 | 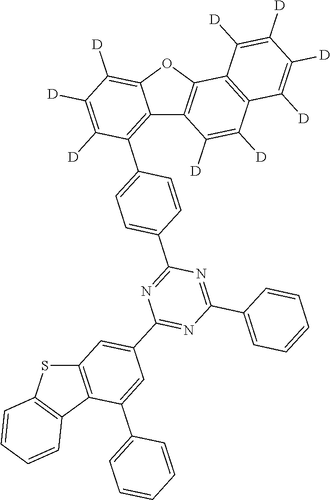 [SMILES: [2H]c1c([2H])c(-c2ccc(-c3nc(-c4ccccc4)nc(-c4cc(-c5ccccc5)c5c(c4)sc4ccccc45)n3)cc2)c2c(oc3c4c([2H])c([2H])c([2H])c([2H])c4c([2H])c([2H])c32)c1[2H]] | 76 |
| 1-118 | 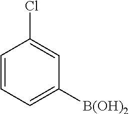 | 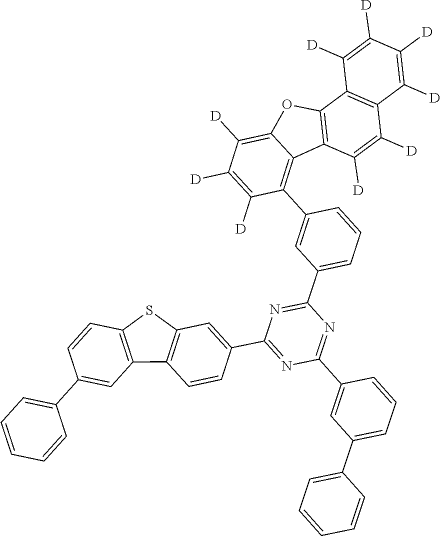 [SMILES: [2H]c1c([2H])c(-c2cccc(-c3nc(-c4cccc(-c5ccccc5)c4)nc(-c4ccc5c(c4)sc4ccc(-c6ccccc6)cc45)n3)c2)c2c(oc3c4c([2H])c([2H])c([2H])c([2H])c4c([2H])c([2H])c32)c1[2H]] | 77 |
| 1-119 | 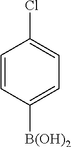 | 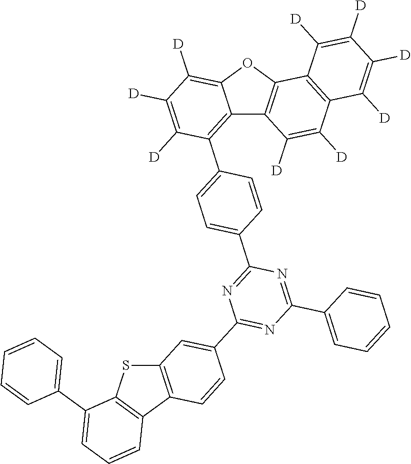 [SMILES: [2H]c1c([2H])c(-c2ccc(-c3nc(-c4ccccc4)nc(-c4ccc5c(c4)sc4c(-c6ccccc6)cccc45)n3)cc2)c2c(oc3c4c([2H])c([2H])c([2H])c([2H])c4c([2H])c([2H])c32)c1[2H]] | 80 |
| 1-120 | 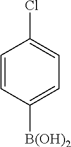 | 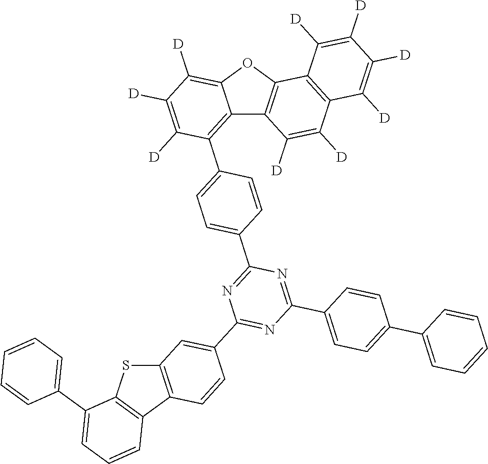 [SMILES: [2H]c1c([2H])c(-c2ccc(-c3nc(-c4ccc(-c5ccccc5)cc4)nc(-c4ccc5c(c4)sc4c(-c6ccccc6)cccc45)n3)cc2)c2c(oc3c4c([2H])c([2H])c([2H])c([2H])c4c([2H])c([2H])c32)c1[2H]] | 76 |
| 1-133 | 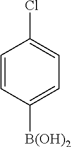 | 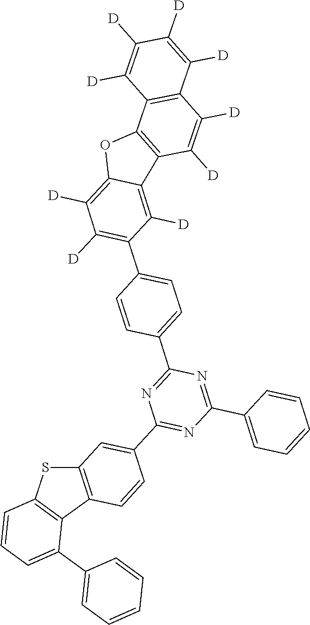 [SMILES: [2H]c1c(-c2ccc(-c3nc(-c4ccccc4)nc(-c4ccc5c(c4)sc4cccc(-c6ccccc6)c45)n3)cc2)c([2H])c2c(oc3c4c([2H])c([2H])c([2H])c([2H])c4c([2H])c([2H])c32)c1[2H]] | 79 |
| 1-134 | 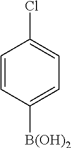 | 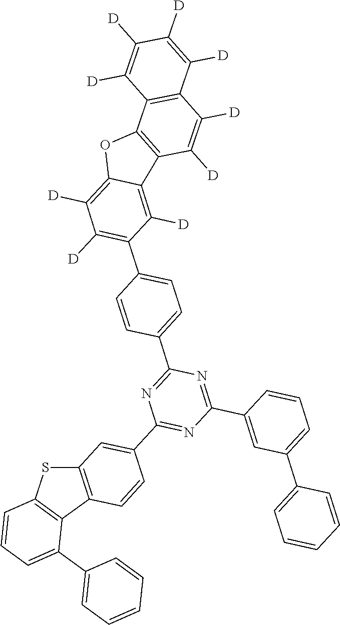 [SMILES: [2H]c1c(-c2ccc(-c3nc(-c4cccc(-c5ccccc5)c4)nc(-c4ccc5c(c4)sc4cccc(-c6ccccc6)c45)n3)cc2)c([2H])c2c(oc3c4c([2H])c([2H])c([2H])c([2H])c4c([2H])c([2H])c32)c1[2H]] | 80 |
| 1-135 | 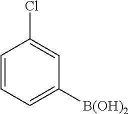 | 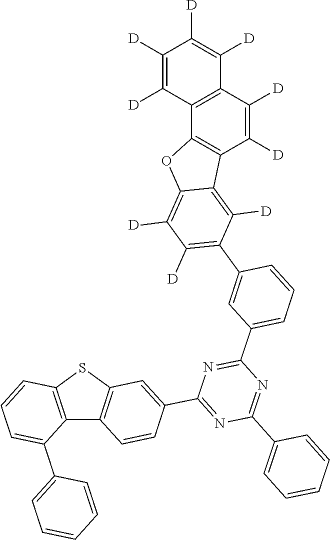 [SMILES: [2H]c1c(-c2cccc(-c3nc(-c4ccccc4)nc(-c4ccc5c(c4)sc4cccc(-c6ccccc6)c45)n3)c2)c([2H])c2c(oc3c4c([2H])c([2H])c([2H])c([2H])c4c([2H])c([2H])c32)c1[2H]] | 81 |
| 1-136 | 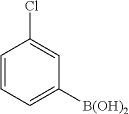 | 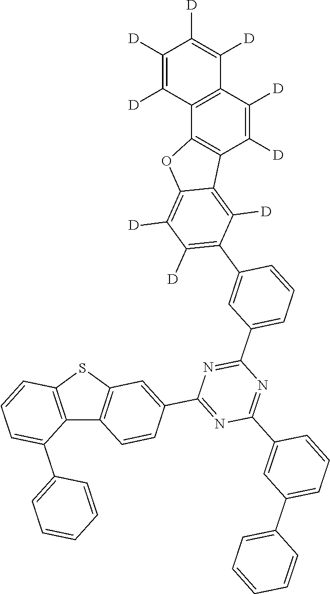 [SMILES: [2H]c1c(-c2cccc(-c3nc(-c4cccc(-c5ccccc5)c4)nc(-c4ccc5c(c4)sc4cccc(-c6ccccc6)c45)n3)c2)c([2H])c2c(oc3c4c([2H])c([2H])c([2H])c([2H])c4c([2H])c([2H])c32)c1[2H]] | 82 |
| 1-137 | 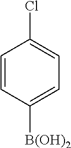 | 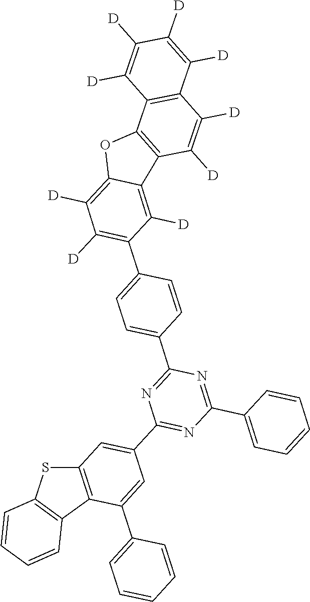 [SMILES: [2H]c1c(-c2ccc(-c3nc(-c4ccccc4)nc(-c4cc(-c5ccccc5)c5c(c4)sc4ccccc45)n3)cc2)c([2H])c2c(oc3c4c([2H])c([2H])c([2H])c([2H])c4c([2H])c([2H])c32)c1[2H]] | 77 |
| 1-138 | 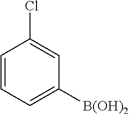 | 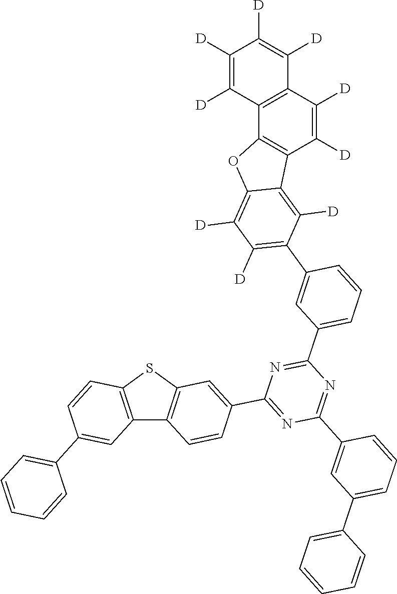 [SMILES: [2H]c1c(-c2cccc(-c3nc(-c4cccc(-c5ccccc5)c4)nc(-c4ccc5c(c4)sc4ccc(-c6ccccc6)cc45)n3)c2)c([2H])c2c(oc3c4c([2H])c([2H])c([2H])c([2H])c4c([2H])c([2H])c32)c1[2H]] | 70 |
| 1-139 | 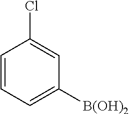 | 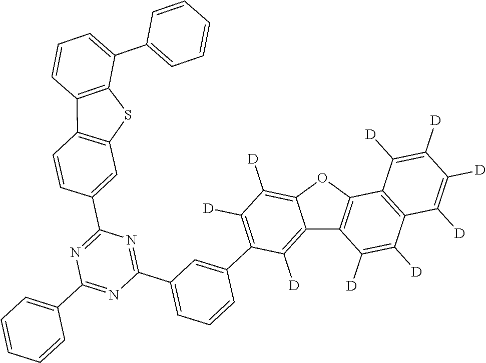 [SMILES: [2H]c1c(-c2cccc(-c3nc(-c4ccccc4)nc(-c4ccc5c(c4)sc4c(-c6ccccc6)cccc45)n3)c2)c([2H])c2c(oc3c4c([2H])c([2H])c([2H])c([2H])c4c([2H])c([2H])c32)c1[2H]] | 79 |
| 1-140 | 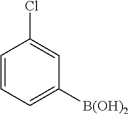 | 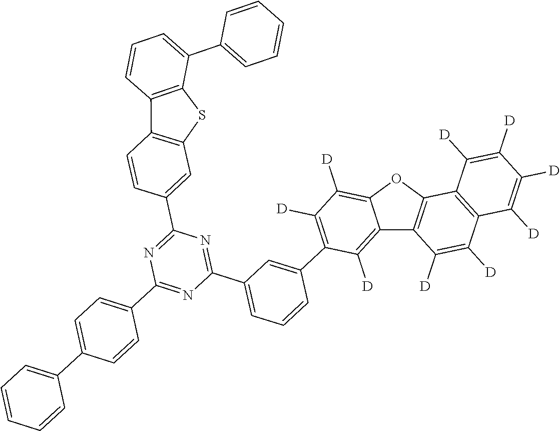 [SMILES: [2H]c1c(-c2cccc(-c3nc(-c4ccc(-c5ccccc5)cc4)nc(-c4ccc5c(c4)sc4c(-c6ccccc6)cccc45)n3)c2)c([2H])c2c(oc3c4c([2H])c([2H])c([2H])c([2H])c4c([2H])c([2H])c32)c1[2H]] | 81 |
| 1-153 | 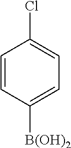 | 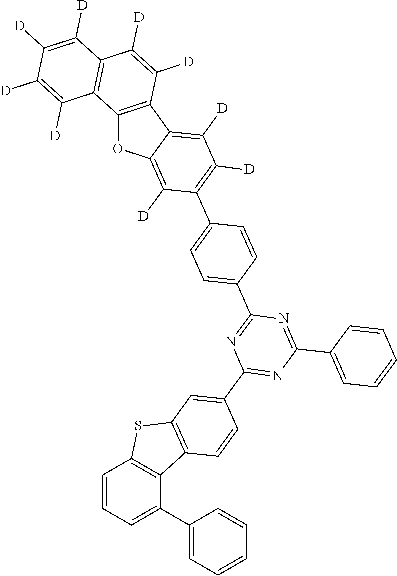 [SMILES: [2H]c1c([2H])c([2H])c2c(c1[2H])c([2H])c([2H])c1c2oc2c([2H])c(-c3ccc(-c4nc(-c5ccccc5)nc(-c5ccc6c(c5)sc5cccc(-c7ccccc7)c56)n4)cc3)c([2H])c([2H])c21] | 82 |
| 1-154 | 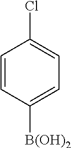 | 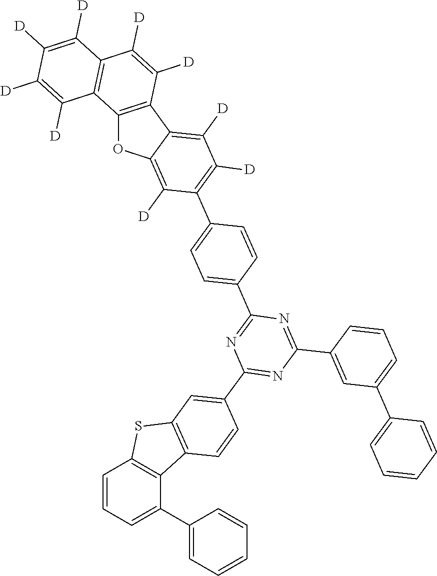 [SMILES: [2H]c1c([2H])c([2H])c2c(c1[2H])c([2H])c([2H])c1c2oc2c([2H])c(-c3ccc(-c4nc(-c5cccc(-c6ccccc6)c5)nc(-c5ccc6c(c5)sc5cccc(-c7ccccc7)c56)n4)cc3)c([2H])c([2H])c21] | 77 |
| 1-155 | 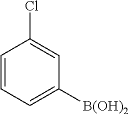 | 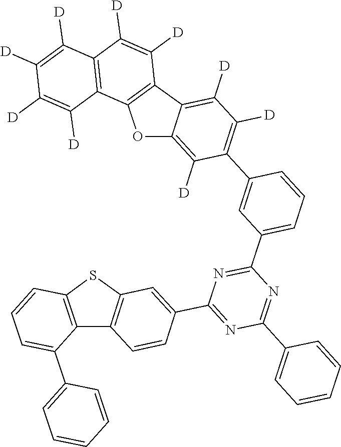 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)sc5cccc(-c7ccccc7)c56)n4)c3)c([2H])c([2H])c21] | 81 |
| 1-156 | 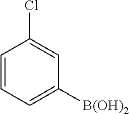 | 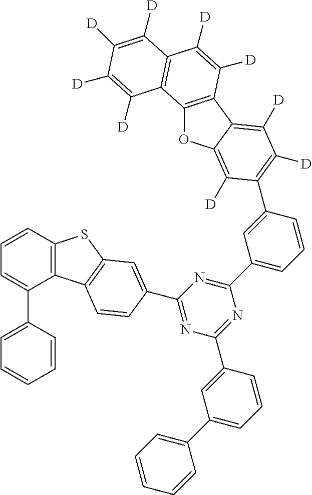 [SMILES: [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)sc5cccc(-c7ccccc7)c56)n4)c3)c([2H])c([2H])c21] | 82 |
| 1-157 | 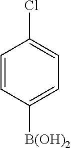 | 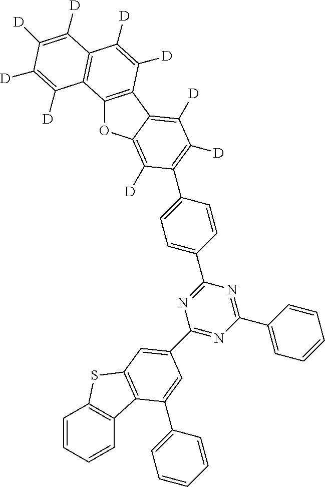 [SMILES: [2H]c1c([2H])c([2H])c2c(c1[2H])c([2H])c([2H])c1c2oc2c([2H])c(-c3ccc(-c4nc(-c5ccccc5)nc(-c5cc(-c6ccccc6)c6c(c5)sc5ccccc56)n4)cc3)c([2H])c([2H])c21] | 76 |
| 1-158 | 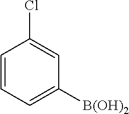 | 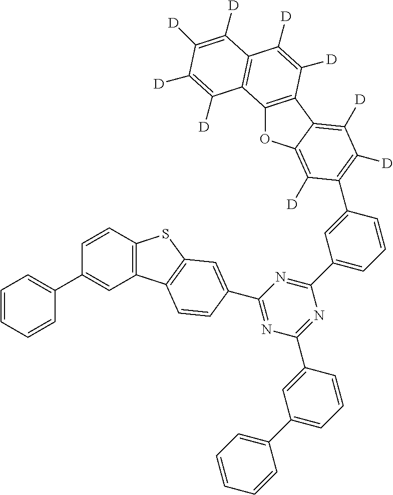 [SMILES: [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)sc5ccc(-c7ccccc7)cc56)n4)c3)c([2H])c([2H])c21] | 79 |
| 1-159 | 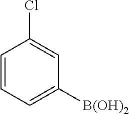 | 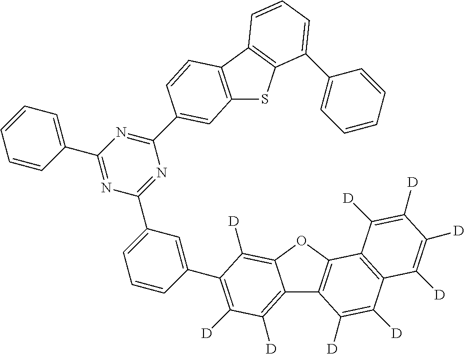 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)sc5c(-c7ccccc7)cccc56)n4)c3)c([2H])c([2H])c21] | 80 |
| 1-160 | 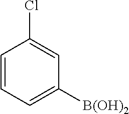 | 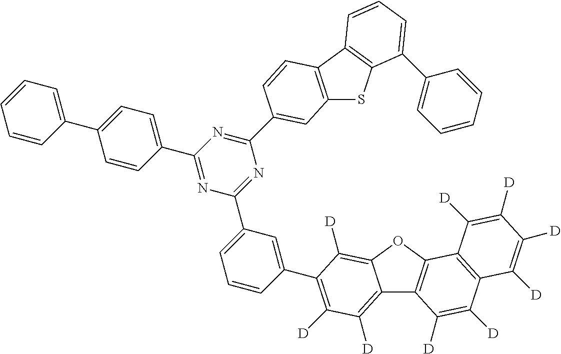 [SMILES: [2H]c1c([2H])c([2H])c2c(c1[2H])c([2H])c([2H])c1c2oc2c([2H])c(-c3cccc(-c4nc(-c5ccc(-c6ccccc6)cc5)nc(-c5ccc6c(c5)sc5c(-c7ccccc7)cccc56)n4)c3)c([2H])c([2H])c21] | 81 |
| 1-173 | 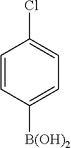 | 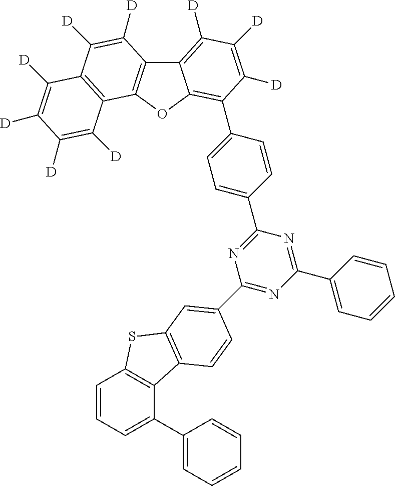 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3ccc4c(c3)sc3cccc(-c5ccccc5)c34)n2)cc1] | 82 |
| 1-174 | 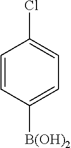 | 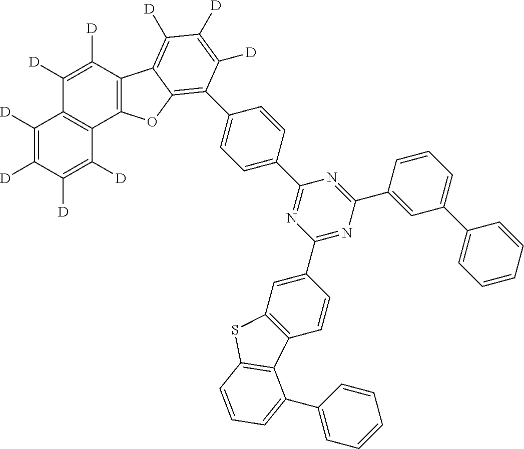 [SMILES: [2H]c1c([2H])c([2H])c2c(oc3c4c([2H])c([2H])c([2H])c([2H])c4c([2H])c([2H])c32)c1-c1ccc(-c2nc(-c3cccc(-c4ccccc4)c3)nc(-c3ccc4c(c3)sc3cccc(-c5ccccc5)c34)n2)cc1] | 77 |
| 1-175 | 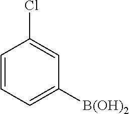 | 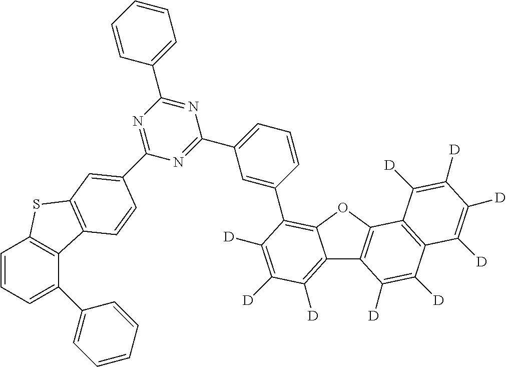 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)sc3cccc(-c5ccccc5)c34)n2)c1] | 70 |
| 1-176 | 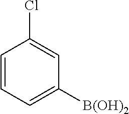 | 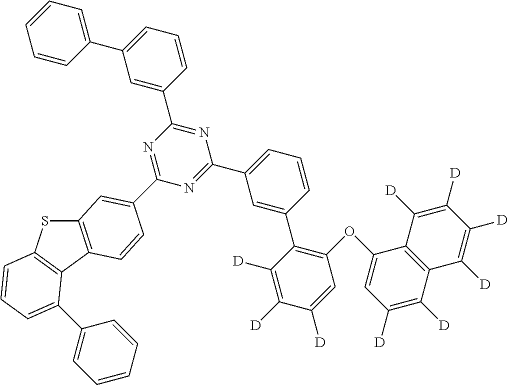 [SMILES: [2H]c1cc(Oc2cc([2H])c([2H])c3c([2H])c([2H])c([2H])c([2H])c23)c(-c2cccc(-c3nc(-c4cccc(-c5ccccc5)c4)nc(-c4ccc5c(c4)sc4cccc(-c6ccccc6)c45)n3)c2)c([2H])c1[2H]] | 79 |
| 1-177 | 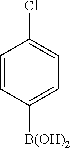 | 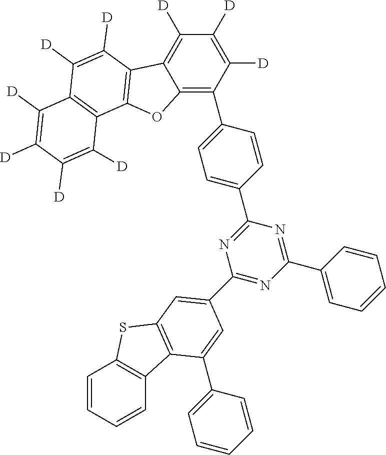 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3cc(-c4ccccc4)c4c(c3)sc3ccccc34)n2)cc1] | 81 |
| 1-178 | 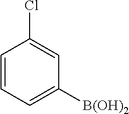 | 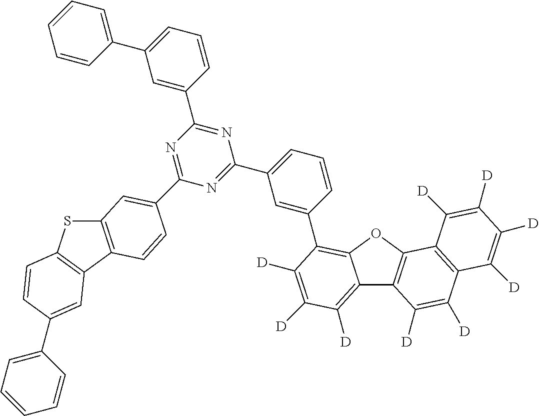 [SMILES: [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3ccc(-c5ccccc5)cc34)n2)c1] | 82 |
| 1-179 | 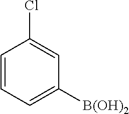 | 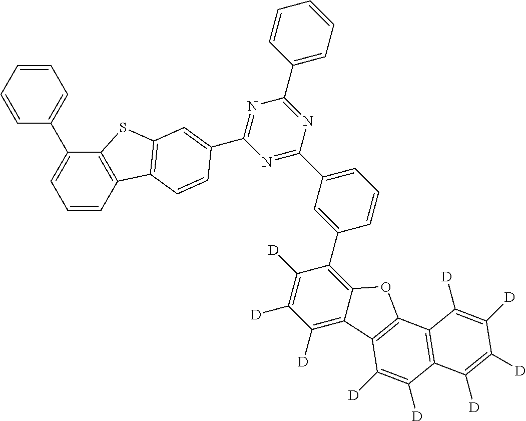 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)sc3c(-c5ccccc5)cccc34)n2)c1] | 77 |
| 1-180 | 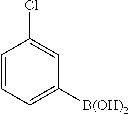 | 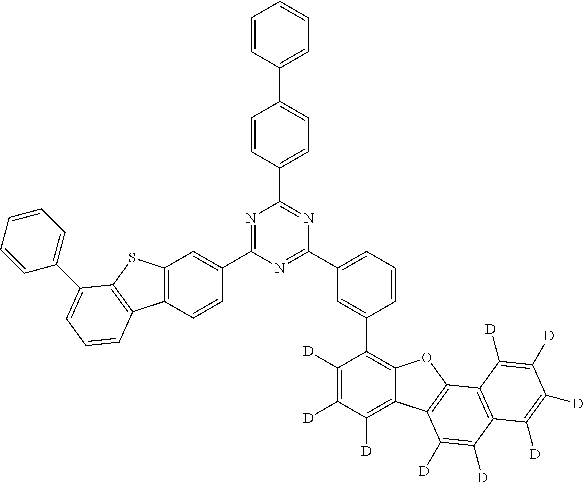 [SMILES: [2H]c1c([2H])c([2H])c2c(oc3c4c([2H])c([2H])c([2H])c([2H])c4c([2H])c([2H])c32)c1-c1cccc(-c2nc(-c3ccc(-c4ccccc4)cc3)nc(-c3ccc4c(c3)sc3c(-c5ccccc5)cccc34)n2)c1] | 81 |
| 1-193 | 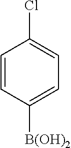 | 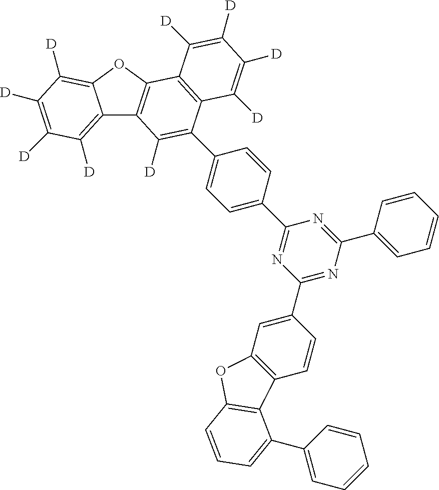 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H]] | 76 |
| 1-194 | 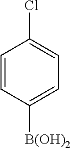 | 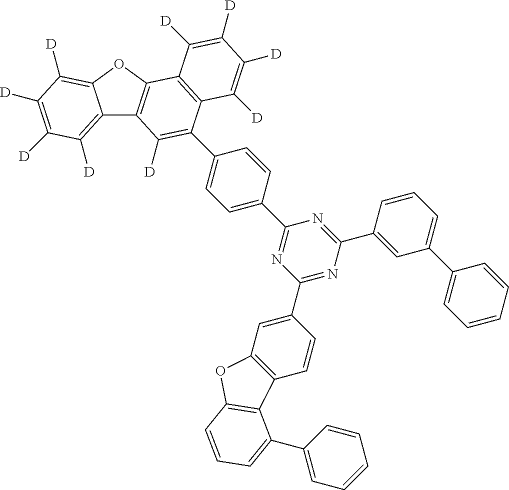 [SMILES: [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]] | 79 |
| 1-195 | 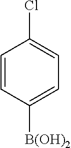 | 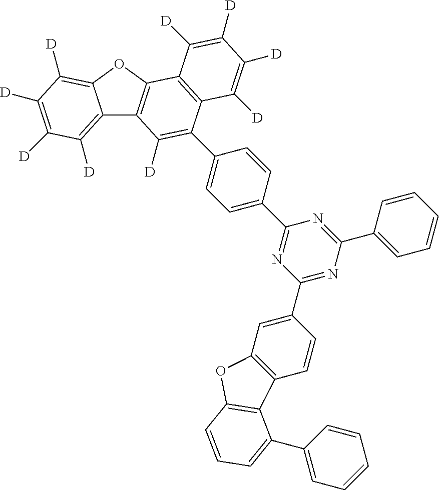 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H]] | 80 |
| 1-196 | 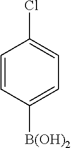 | 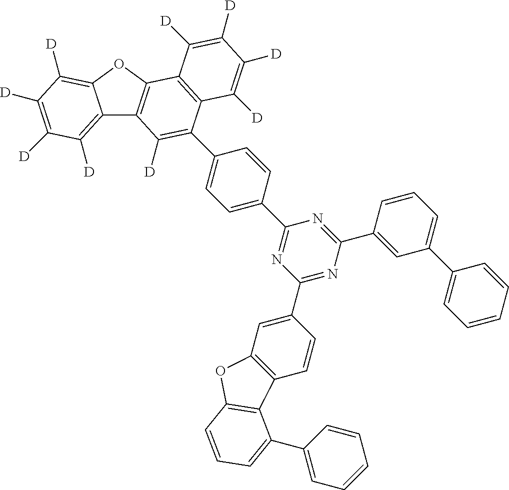 [SMILES: [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]] | 81 |
| 1-197 | 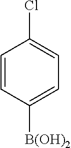 | 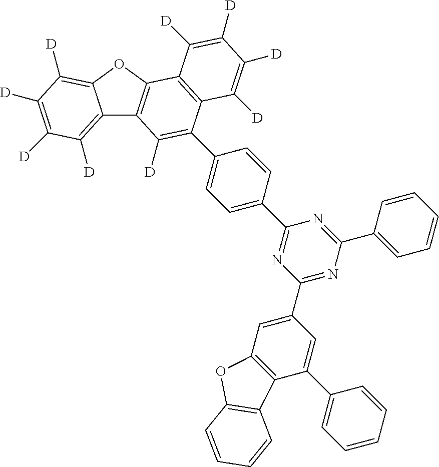 [SMILES: [2H]c1c([2H])c([2H])c2c(oc3c4c([2H])c([2H])c([2H])c([2H])c4c(-c4ccc(-c5nc(-c6ccccc6)nc(-c6cc(-c7ccccc7)c7c(c6)oc6ccccc67)n5)cc4)c([2H])c32)c1[2H]] | 82 |
| 1-198 | 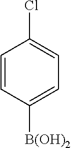 | 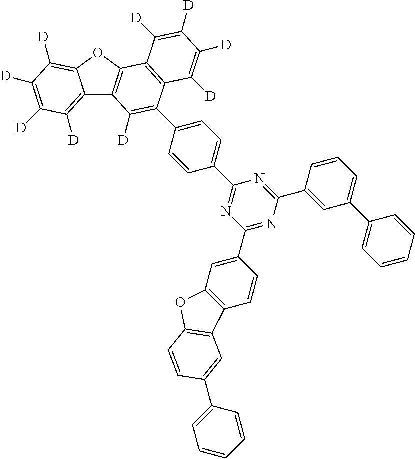 [SMILES: [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6ccc(-c8ccccc8)cc67)n5)cc4)c([2H])c32)c1[2H]] | 77 |
| 1-199 | 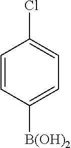 | 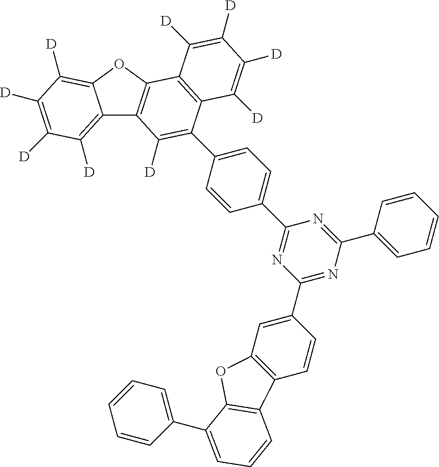 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6c(-c8ccccc8)cccc67)n5)cc4)c([2H])c32)c1[2H]] | 70 |
| 1-200 | 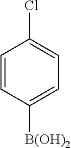 | 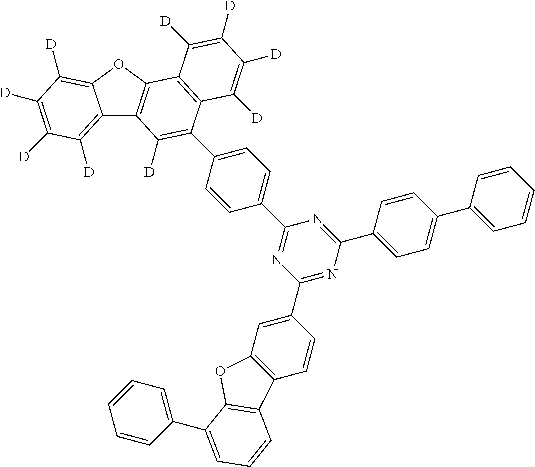 [SMILES: [2H]c1c([2H])c([2H])c2c(oc3c4c([2H])c([2H])c([2H])c([2H])c4c(-c4ccc(-c5nc(-c6ccc(-c7ccccc7)cc6)nc(-c6ccc7c(c6)oc6c(-c8ccccc8)cccc67)n5)cc4)c([2H])c32)c1[2H]] | 79 |
| 1-209 | 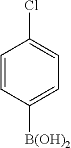 | 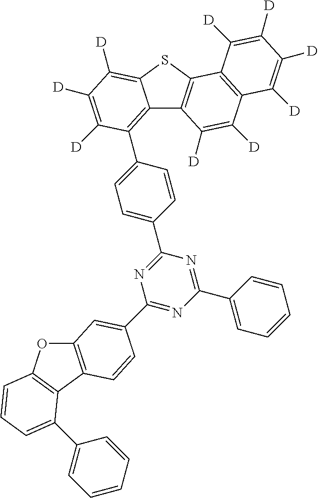 [SMILES: [2H]c1c([2H])c(-c2ccc(-c3nc(-c4ccccc4)nc(-c4ccc5c(c4)oc4cccc(-c6ccccc6)c45)n3)cc2)c2c(sc3c4c([2H])c([2H])c([2H])c([2H])c4c([2H])c([2H])c32)c1[2H]] | 81 |
| 1-210 | 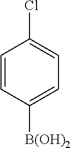 | 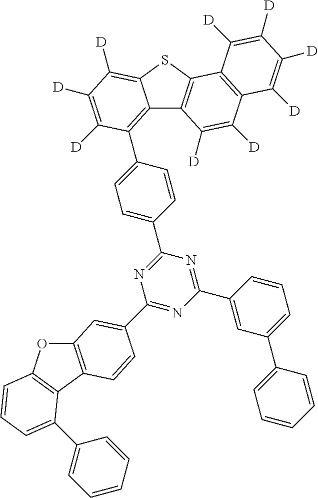 [SMILES: [2H]c1c([2H])c(-c2ccc(-c3nc(-c4cccc(-c5ccccc5)c4)nc(-c4ccc5c(c4)oc4cccc(-c6ccccc6)c45)n3)cc2)c2c(sc3c4c([2H])c([2H])c([2H])c([2H])c4c([2H])c([2H])c32)c1[2H]] | 82 |
| 1-211 | 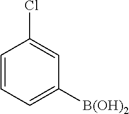 | 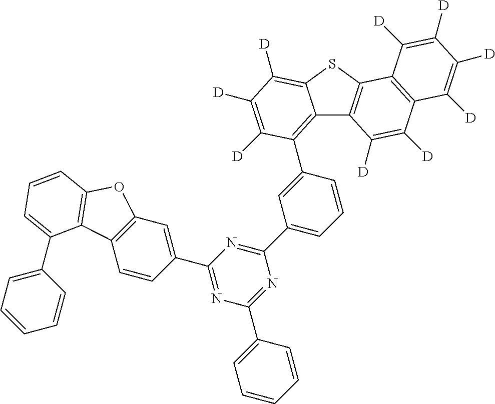 [SMILES: [2H]c1c([2H])c(-c2cccc(-c3nc(-c4ccccc4)nc(-c4ccc5c(c4)oc4cccc(-c6ccccc6)c45)n3)c2)c2c(sc3c4c([2H])c([2H])c([2H])c([2H])c4c([2H])c([2H])c32)c1[2H]] | 77 |
| 1-212 | 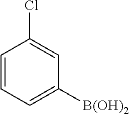 | 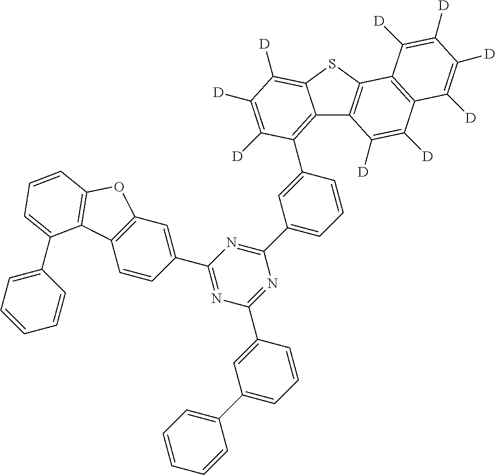 [SMILES: [2H]c1c([2H])c(-c2cccc(-c3nc(-c4cccc(-c5ccccc5)c4)nc(-c4ccc5c(c4)oc4cccc(-c6ccccc6)c45)n3)c2)c2c(sc3c4c([2H])c([2H])c([2H])c([2H])c4c([2H])c([2H])c32)c1[2H]] | 81 |
| 1-219 | 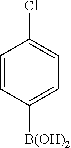 | 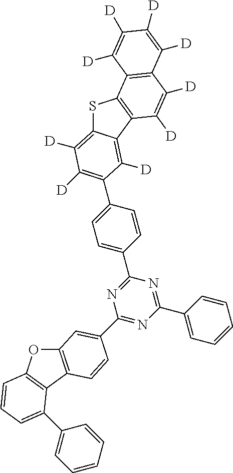 [SMILES: [2H]c1c(-c2ccc(-c3nc(-c4ccccc4)nc(-c4ccc5c(c4)oc4cccc(-c6ccccc6)c45)n3)cc2)c([2H])c2c(sc3c4c([2H])c([2H])c([2H])c([2H])c4c([2H])c([2H])c32)c1[2H]] | 82 |
| 1-220 | 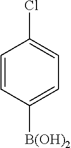 | 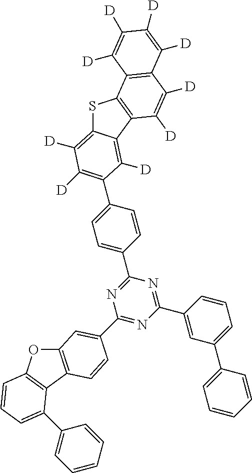 [SMILES: [2H]c1c(-c2ccc(-c3nc(-c4cccc(-c5ccccc5)c4)nc(-c4ccc5c(c4)oc4cccc(-c6ccccc6)c45)n3)cc2)c([2H])c2c(sc3c4c([2H])c([2H])c([2H])c([2H])c4c([2H])c([2H])c32)c1[2H]] | 80 |
| 1-225 | 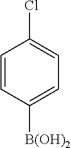 | 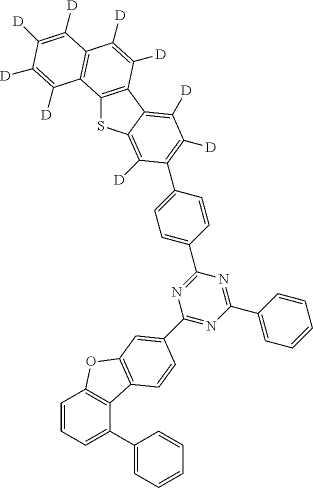 [SMILES: [2H]c1c([2H])c([2H])c2c(c1[2H])c([2H])c([2H])c1c2sc2c([2H])c(-c3ccc(-c4nc(-c5ccccc5)nc(-c5ccc6c(c5)oc5cccc(-c7ccccc7)c56)n4)cc3)c([2H])c([2H])c21] | 82 |
| 1-226 | 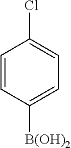 | 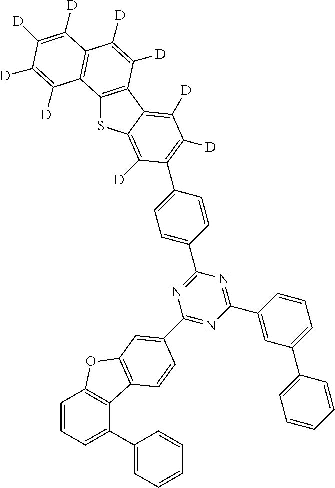 [SMILES: [2H]c1c([2H])c([2H])c2c(c1[2H])c([2H])c([2H])c1c2sc2c([2H])c(-c3ccc(-c4nc(-c5cccc(-c6ccccc6)c5)nc(-c5ccc6c(c5)oc5cccc(-c7ccccc7)c56)n4)cc3)c([2H])c([2H])c21] | 78 |
| 1-227 | 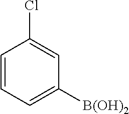 | 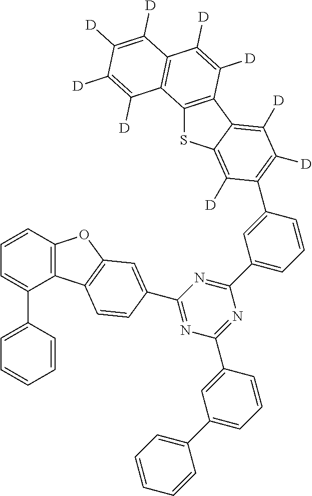 [SMILES: [2H]c1c([2H])c([2H])c2c(c1[2H])c([2H])c([2H])c1c2sc2c([2H])c(-c3cccc(-c4nc(-c5cccc(-c6ccccc6)c5)nc(-c5ccc6c(c5)oc5cccc(-c7ccccc7)c56)n4)c3)c([2H])c([2H])c21] | 77 |
| 1-228 | 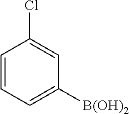 | 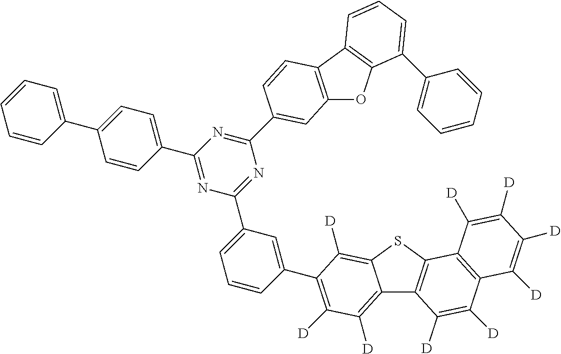 [SMILES: [2H]c1c([2H])c([2H])c2c(c1[2H])c([2H])c([2H])c1c2sc2c([2H])c(-c3cccc(-c4nc(-c5ccc(-c6ccccc6)cc5)nc(-c5ccc6c(c5)oc5c(-c7ccccc7)cccc56)n4)c3)c([2H])c([2H])c21] | 85 |
| 1-233 | 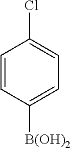 | 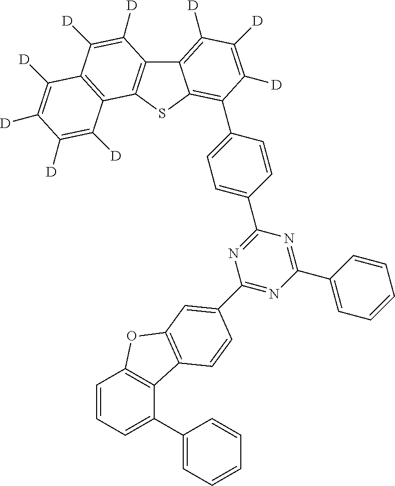 [SMILES: [2H]c1c([2H])c([2H])c2c(sc3c4c([2H])c([2H])c([2H])c([2H])c4c([2H])c([2H])c32)c1-c1ccc(-c2nc(-c3ccccc3)nc(-c3ccc4c(c3)oc3cccc(-c5ccccc5)c34)n2)cc1] | 71 |
| 1-234 | 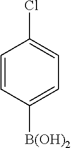 | 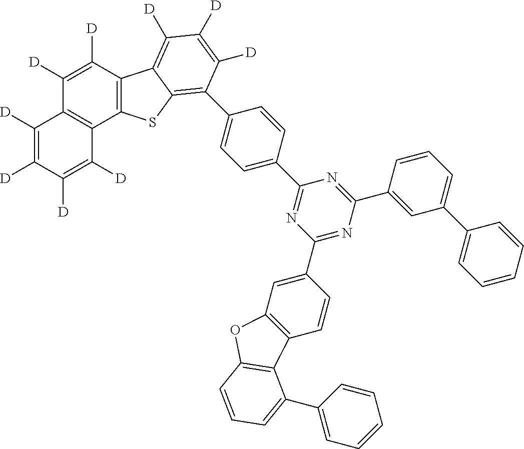 [SMILES: [2H]c1c([2H])c([2H])c2c(sc3c4c([2H])c([2H])c([2H])c([2H])c4c([2H])c([2H])c32)c1-c1ccc(-c2nc(-c3cccc(-c4ccccc4)c3)nc(-c3ccc4c(c3)oc3cccc(-c5ccccc5)c34)n2)cc1] | 74 |
| 1-235 | 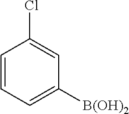 | 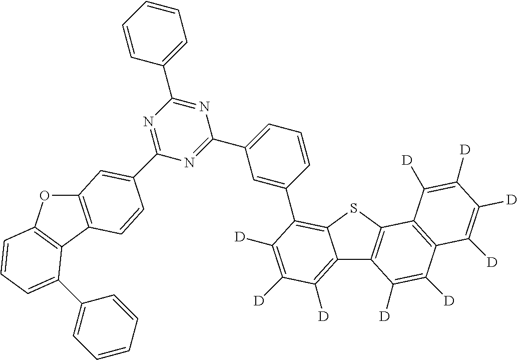 [SMILES: [2H]c1c([2H])c([2H])c2c(sc3c4c([2H])c([2H])c([2H])c([2H])c4c([2H])c([2H])c32)c1-c1cccc(-c2nc(-c3ccccc3)nc(-c3ccc4c(c3)oc3cccc(-c5ccccc5)c34)n2)c1] | 73 |
| 1-236 | 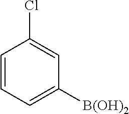 | 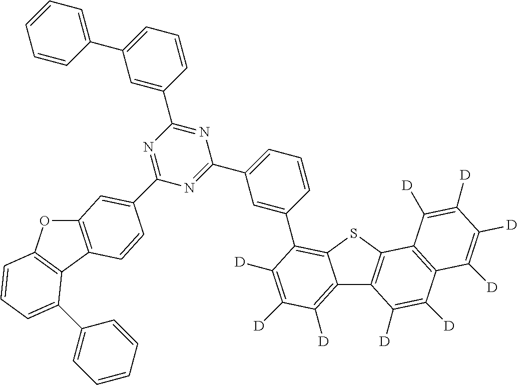 [SMILES: [2H]c1c([2H])c([2H])c2c(sc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3cccc(-c5ccccc5)c34)n2)c1] | 76 |
| 1-239 | 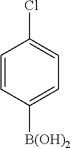 | 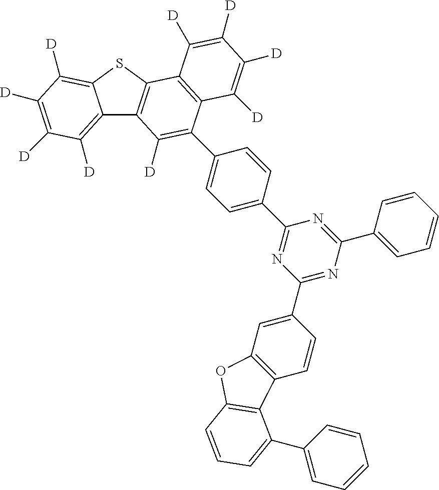 [SMILES: [2H]c1c([2H])c([2H])c2c(sc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H]] | 82 |
| 1-240 | 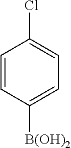 | 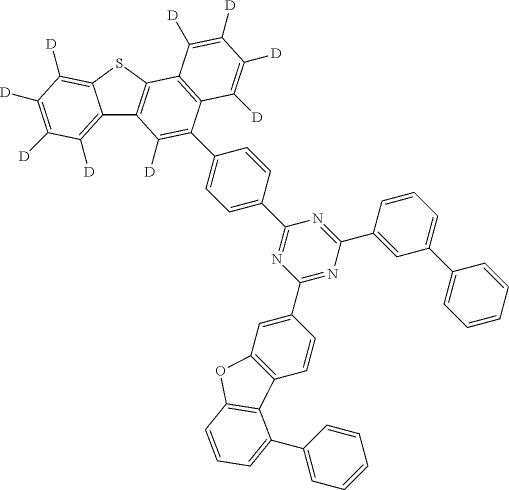 [SMILES: [2H]c1c([2H])c([2H])c2c(sc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]] | 78 |
| 1-245 | 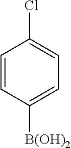 | 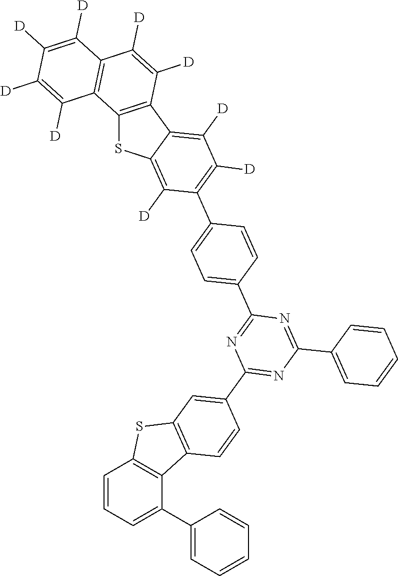 [SMILES: [2H]c1c([2H])c([2H])c2c(c1[2H])c([2H])c([2H])c1c2sc2c([2H])c(-c3ccc(-c4nc(-c5ccccc5)nc(-c5ccc6c(c5)sc5cccc(-c7ccccc7)c56)n4)cc3)c([2H])c([2H])c21] | 77 |
| 1-246 | 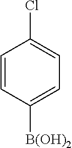 | 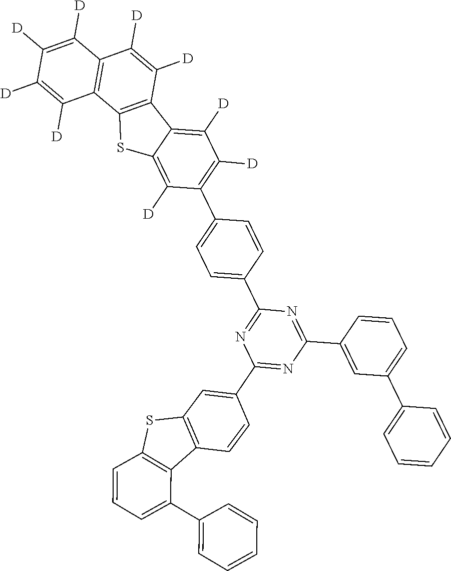 [SMILES: [2H]c1c([2H])c([2H])c2c(c1[2H])c([2H])c([2H])c1c2sc2c([2H])c(-c3ccc(-c4nc(-c5cccc(-c6ccccc6)c5)nc(-c5ccc6c(c5)sc5cccc(-c7ccccc7)c56)n4)cc3)c([2H])c([2H])c21] | 76 |
| 1-247 | 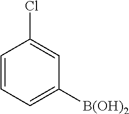 | 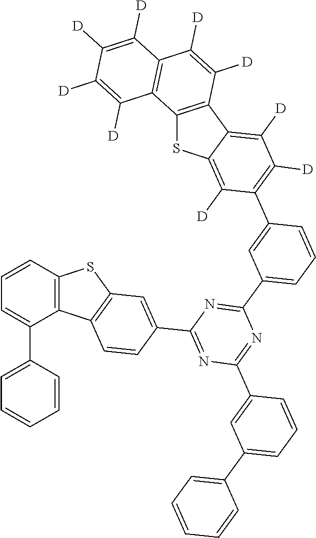 [SMILES: [2H]c1c([2H])c([2H])c2c(c1[2H])c([2H])c([2H])c1c2sc2c([2H])c(-c3cccc(-c4nc(-c5cccc(-c6ccccc6)c5)nc(-c5ccc6c(c5)sc5cccc(-c7ccccc7)c56)n4)c3)c([2H])c([2H])c21] | 75 |
| 1-248 | 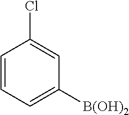 | 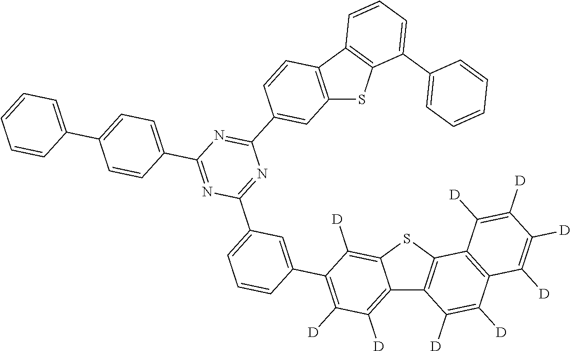 [SMILES: [2H]c1c([2H])c([2H])c2c(c1[2H])c([2H])c([2H])c1c2sc2c([2H])c(-c3cccc(-c4nc(-c5ccc(-c6ccccc6)cc5)nc(-c5ccc6c(c5)sc5c(-c7ccccc7)cccc56)n4)c3)c([2H])c([2H])c21] | 77 |
| 1-253 | 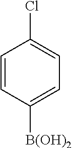 | 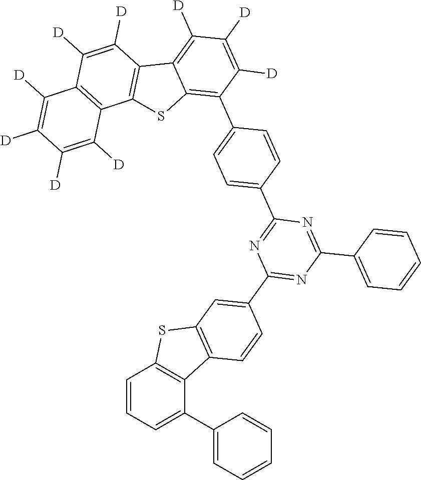 [SMILES: [2H]c1c([2H])c([2H])c2c(sc3c4c([2H])c([2H])c([2H])c([2H])c4c([2H])c([2H])c32)c1-c1ccc(-c2nc(-c3ccccc3)nc(-c3ccc4c(c3)sc3cccc(-c5ccccc5)c34)n2)cc1] | 73 |
| 1-254 | 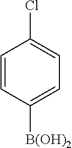 | 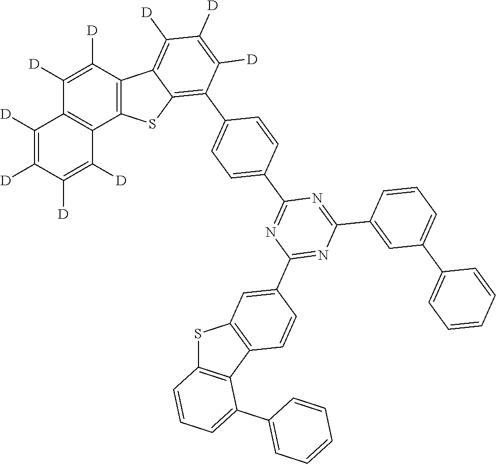 [SMILES: [2H]c1c([2H])c([2H])c2c(sc3c4c([2H])c([2H])c([2H])c([2H])c4c([2H])c([2H])c32)c1-c1ccc(-c2nc(-c3cccc(-c4ccccc4)c3)nc(-c3ccc4c(c3)sc3cccc(-c5ccccc5)c34)n2)cc1] | 84 |
| 1-255 | 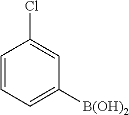 | 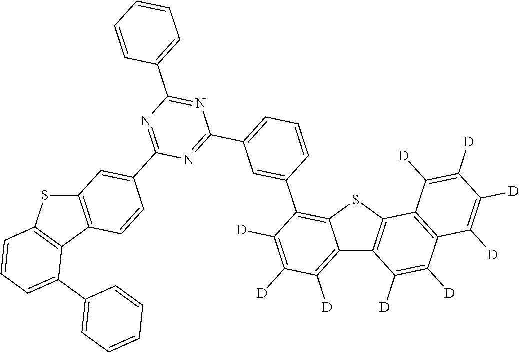 [SMILES: [2H]c1c([2H])c([2H])c2c(sc3c4c([2H])c([2H])c([2H])c([2H])c4c([2H])c([2H])c32)c1-c1cccc(-c2nc(-c3ccccc3)nc(-c3ccc4c(c3)sc3cccc(-c5ccccc5)c34)n2)c1] | 82 |
| 1-256 | 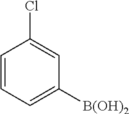 | 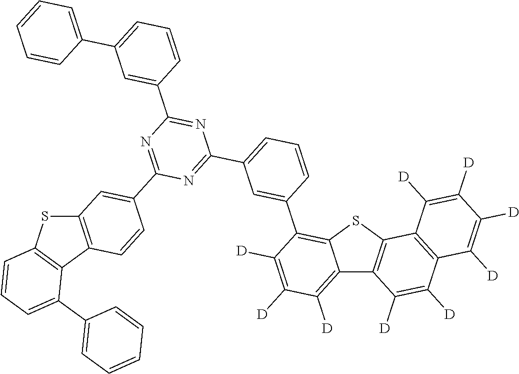 [SMILES: [2H]c1c([2H])c([2H])c2c(sc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3cccc(-c5ccccc5)c34)n2)c1] | 81 |
| 1-259 | 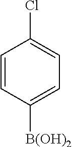 | 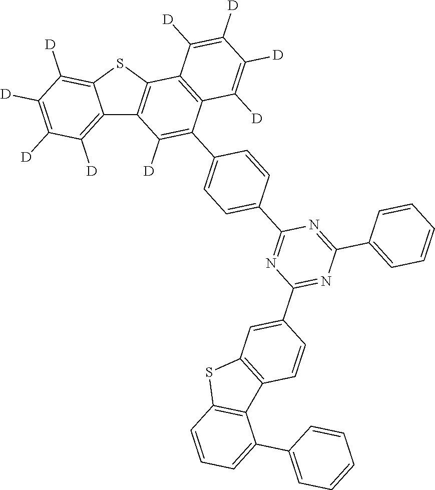 [SMILES: [2H]c1c([2H])c([2H])c2c(sc3c4c([2H])c([2H])c([2H])c([2H])c4c(-c4ccc(-c5nc(-c6ccccc6)nc(-c6ccc7c(c6)sc6cccc(-c8ccccc8)c67)n5)cc4)c([2H])c32)c1[2H]] | 80 |
| 1-260 | 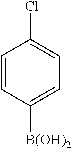 | 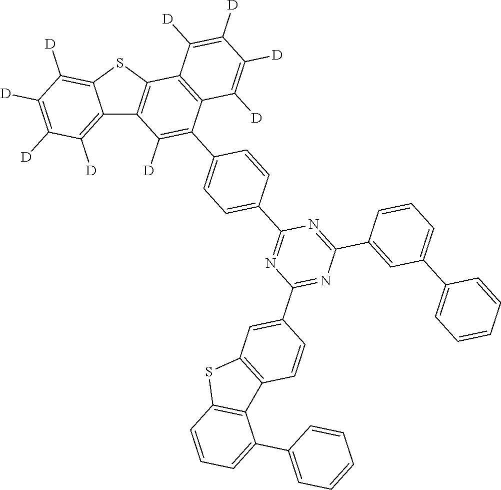 [SMILES: [2H]c1c([2H])c([2H])c2c(sc3c4c([2H])c([2H])c([2H])c([2H])c4c(-c4ccc(-c5nc(-c6cccc(-c7ccccc7)c6)nc(-c6ccc7c(c6)sc6cccc(-c8ccccc8)c67)n5)cc4)c([2H])c32)c1[2H]] | 77 |

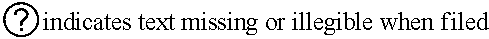

### [Preparation Example 3] Preparation of Compound 1-281

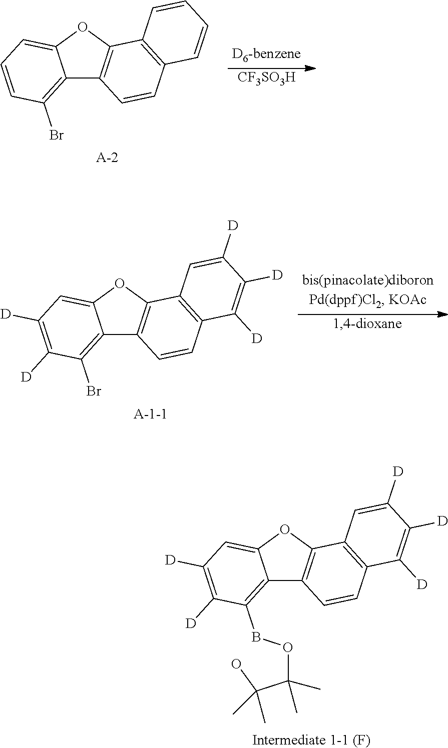

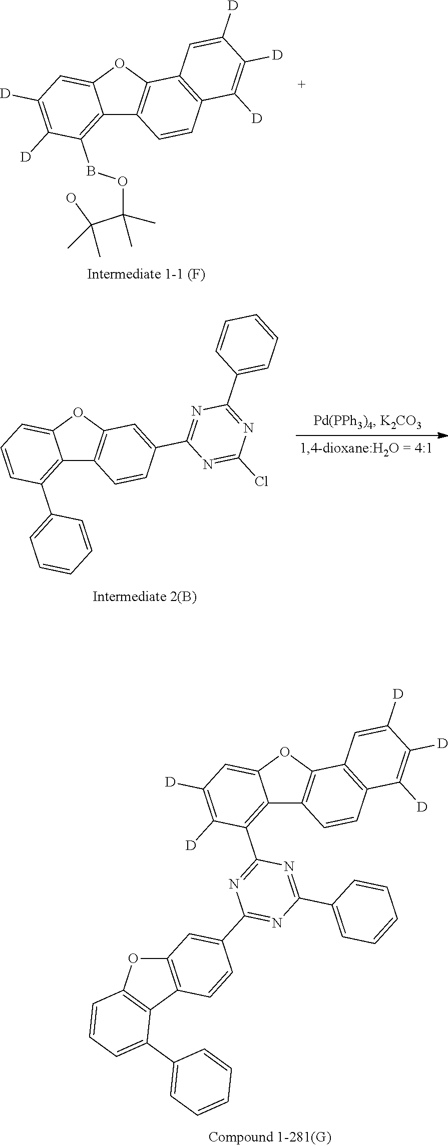

### Synthesis of A-1-1

7-bromonaphtho[1,2-b]benzofuran (A-2) (10 g, 1 eq.), D6-benzene (500 g, 5941.7 mmol.), and CF₃SO₃H (255 g, 1613 mmol) were put into a reaction flask and reacted at 25° C. for 10 hours. After completing the reaction, H₂O was added for neutralization, MC and H₂O were added for extraction, and an organic layer was purified using a column, thereby obtaining Compound A-1-1 (6.9 g, 74%).

In Preparation Example 3, desired Compound (G) was synthesized in the same manner as in Preparation Example 1, except that Reagents A-2, B-4 and B-1 in Table 3 below were used.

TABLE 3

| Com | | | | | Yield |
|---|---|---|---|---|---|
| pound | A-2 | B-4 | B-1 | Compound_{G} | (%) |
| 1- 282 | 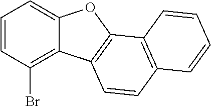 [SMILES: Brc1cccc2oc3c4ccccc4ccc3c12] | 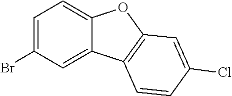 [SMILES: Clc1ccc2c(c1)oc1ccc(Br)cc12] | 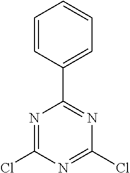 [SMILES: Clc1nc(Cl)nc(-c2ccccc2)n1] | 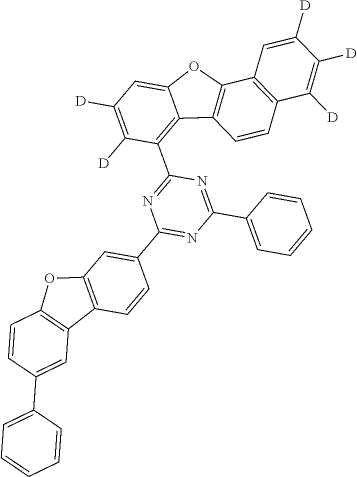 [SMILES: [2H]c1cc2c(ccc3c2oc2cc([2H])c([2H])c(-c4nc(-c5ccccc5)nc(-c5ccc6c(c5)oc5ccc(-c7ccccc7)cc56)n4)c23)c([2H])c1[2H]] | 83 |
| 1- 283 | 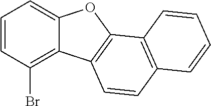 [SMILES: Brc1cccc2oc3c4ccccc4ccc3c12] | 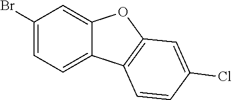 [SMILES: Clc1ccc2c(c1)oc1cc(Br)ccc12] | 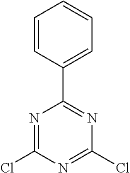 [SMILES: Clc1nc(Cl)nc(-c2ccccc2)n1] | 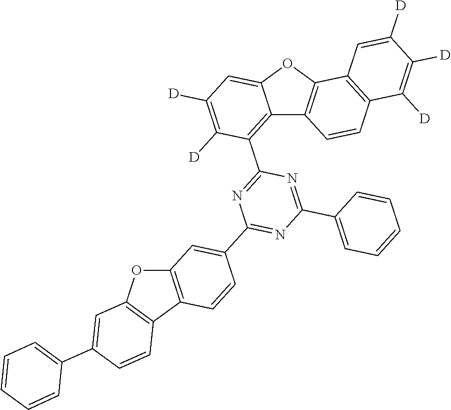 [SMILES: [2H]c1cc2c(ccc3c2oc2cc([2H])c([2H])c(-c4nc(-c5ccccc5)nc(-c5ccc6c(c5)oc5cc(-c7ccccc7)ccc56)n4)c23)c([2H])c1[2H]] | 84 |
| 1- 284 | 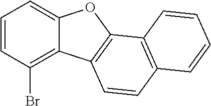 [SMILES: Brc1cccc2oc3c4ccccc4ccc3c12] | 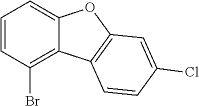 [SMILES: Clc1ccc2c(c1)oc1cccc(Br)c12] | 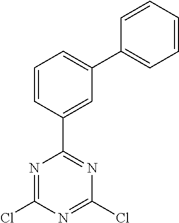 [SMILES: Clc1nc(Cl)nc(-c2cccc(-c3ccccc3)c2)n1] | 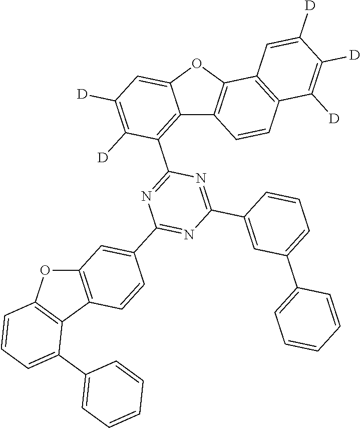 [SMILES: [2H]c1cc2c(ccc3c2oc2cc([2H])c([2H])c(-c4nc(-c5cccc(-c6ccccc6)c5)nc(-c5ccc6c(c5)oc5cccc(-c7ccccc7)c56)n4)c23)c([2H])c1[2H]] | 83 |
| 1- 301 | 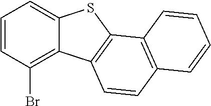 [SMILES: Brc1cccc2sc3c4ccccc4ccc3c12] | 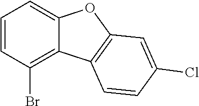 [SMILES: Clc1ccc2c(c1)oc1cccc(Br)c12] | 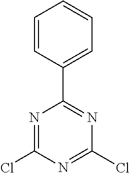 [SMILES: Clc1nc(Cl)nc(-c2ccccc2)n1] | 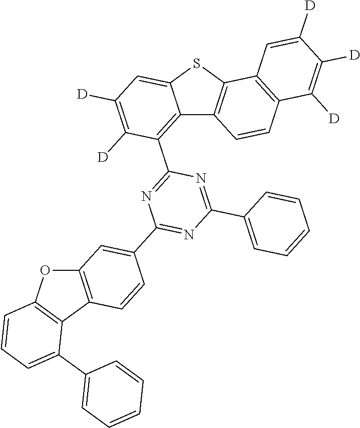 [SMILES: [2H]c1cc2c(ccc3c2sc2cc([2H])c([2H])c(-c4nc(-c5ccccc5)nc(-c5ccc6c(c5)oc5cccc(-c7ccccc7)c56)n4)c23)c([2H])c1[2H]] | 82 |
| 1- 302 | 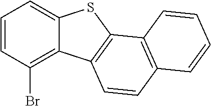 [SMILES: Brc1cccc2sc3c4ccccc4ccc3c12] | 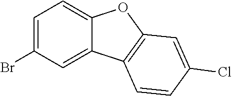 [SMILES: Clc1ccc2c(c1)oc1ccc(Br)cc12] | 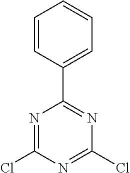 [SMILES: Clc1nc(Cl)nc(-c2ccccc2)n1] | 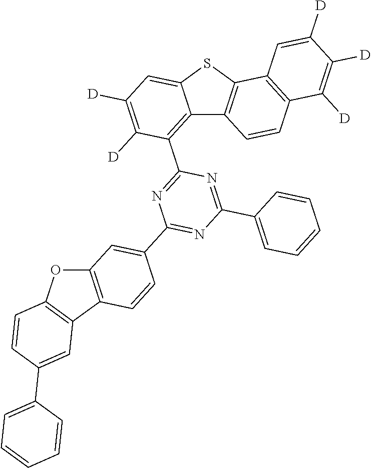 [SMILES: [2H]c1cc2c(ccc3c2sc2cc([2H])c([2H])c(-c4nc(-c5ccccc5)nc(-c5ccc6c(c5)oc5ccc(-c7ccccc7)cc56)n4)c23)c([2H])c1[2H]] | 83 |
| 1- 303 | 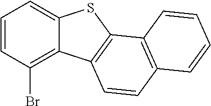 [SMILES: Brc1cccc2sc3c4ccccc4ccc3c12] | 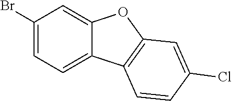 [SMILES: Clc1ccc2c(c1)oc1cc(Br)ccc12] | 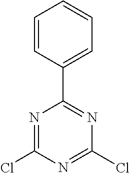 [SMILES: Clc1nc(Cl)nc(-c2ccccc2)n1] | 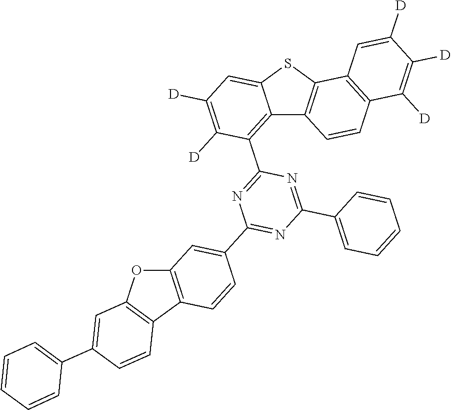 [SMILES: [2H]c1cc2c(ccc3c2sc2cc([2H])c([2H])c(-c4nc(-c5ccccc5)nc(-c5ccc6c(c5)oc5cc(-c7ccccc7)ccc56)n4)c23)c([2H])c1[2H]] | 84 |
| 1- 304 | 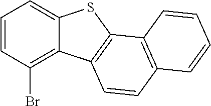 [SMILES: Brc1cccc2sc3c4ccccc4ccc3c12] | 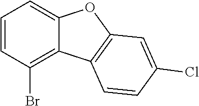 [SMILES: Clc1ccc2c(c1)oc1cccc(Br)c12] | 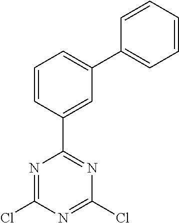 [SMILES: Clc1nc(Cl)nc(-c2cccc(-c3ccccc3)c2)n1] | 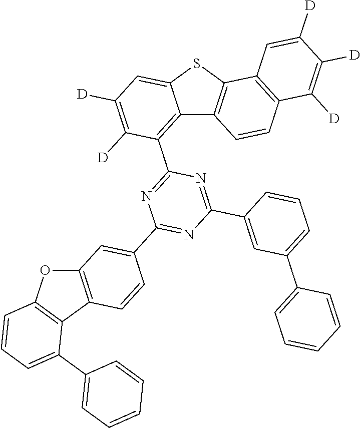 [SMILES: [2H]c1cc2c(ccc3c2sc2cc([2H])c([2H])c(-c4nc(-c5cccc(-c6ccccc6)c5)nc(-c5ccc6c(c5)oc5cccc(-c7ccccc7)c56)n4)c23)c([2H])c1[2H]] | 80 |

### [Preparation Example 4] Preparation of Compound 1-285

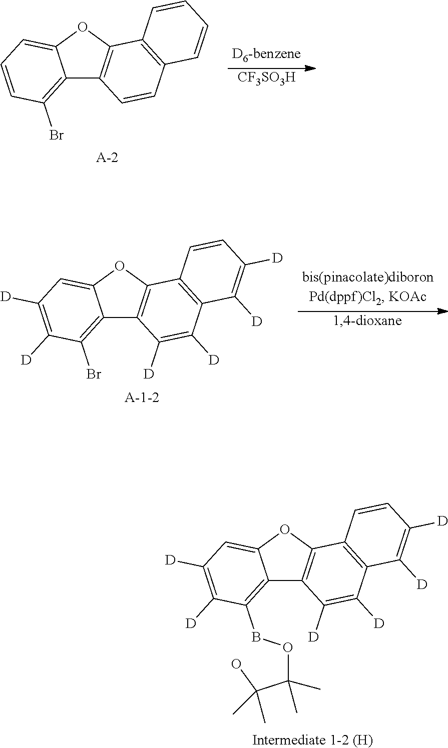

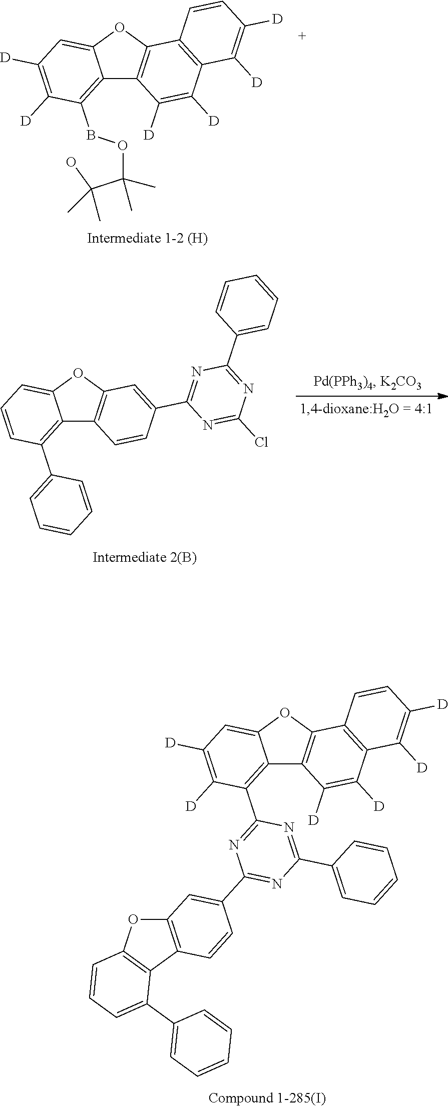

### Synthesis of A-1-2

7-bromonaphtho[1,2-b]benzofuran (A-2)(10 g, 1 eq.), D6-benzene (500 g, 5941.7 mmol.), and CF₃SO₃H (255 g, 1613 mmol) were put into a reaction flask, and reacted at 35° C. for 6 hours. After completing the reaction, H₂O was added for neutralization, MC and H₂O were added for extraction, and an organic layer was purified using a column, thereby obtaining Compound A-1-2 (7.8 g, 72%).

In Preparation Example 4, desired Compound (I) was synthesized in the same manner as in Preparation Example 1, except that Reagents A-2, B-4 and B-1 in Table 4 below were used.

TABLE 4

| Com | | | | | Yield |
|---|---|---|---|---|---|
| pound | A-2 | B-4 | B-1 | Compound¹ | (%) |
| 1- 286 | 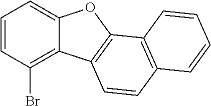 [SMILES: Brc1cccc2oc3c4ccccc4ccc3c12] | 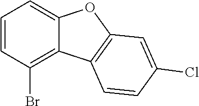 [SMILES: Clc1ccc2c(c1)oc1cccc(Br)c12] | 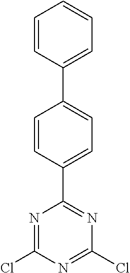 [SMILES: Clc1nc(Cl)nc(-c2ccc(-c3ccccc3)cc2)n1] | 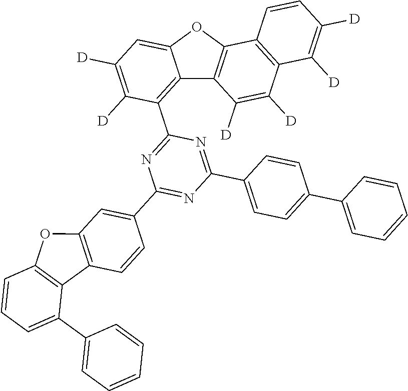 [SMILES: [2H]c1cc2oc3c4ccc([2H])c([2H])c4c([2H])c([2H])c3c2c(-c2nc(-c3ccc(-c4ccccc4)cc3)nc(-c3ccc4c(c3)oc3cccc(-c5ccccc5)c34)n2)c1[2H]] | 80 |
| 1- 287 | 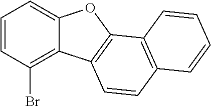 [SMILES: Brc1cccc2oc3c4ccccc4ccc3c12] | 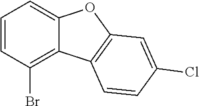 [SMILES: Clc1ccc2c(c1)oc1cccc(Br)c12] | 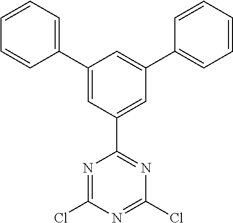 [SMILES: Clc1nc(Cl)nc(-c2cc(-c3ccccc3)cc(-c3ccccc3)c2)n1] | 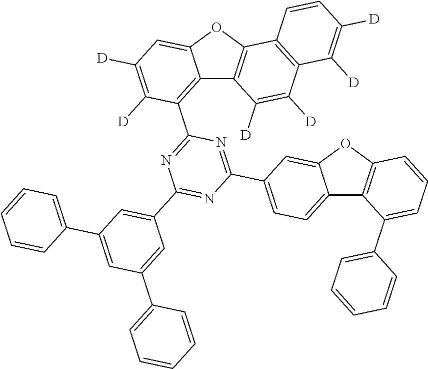 [SMILES: [2H]c1cc2oc3c4ccc([2H])c([2H])c4c([2H])c([2H])c3c2c(-c2nc(-c3cc(-c4ccccc4)cc(-c4ccccc4)c3)nc(-c3ccc4c(c3)oc3cccc(-c5ccccc5)c34)n2)c1[2H]] | 84 |
| 1-288 | 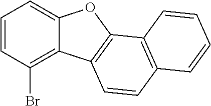 [SMILES: Brc1cccc2oc3c4ccccc4ccc3c12] | 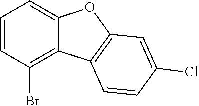 [SMILES: Clc1ccc2c(c1)oc1cccc(Br)c12] | 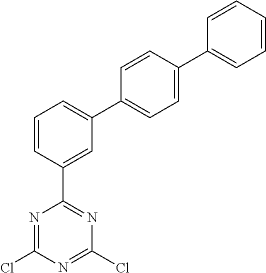 [SMILES: Clc1nc(Cl)nc(-c2cccc(-c3ccc(-c4ccccc4)cc3)c2)n1] | 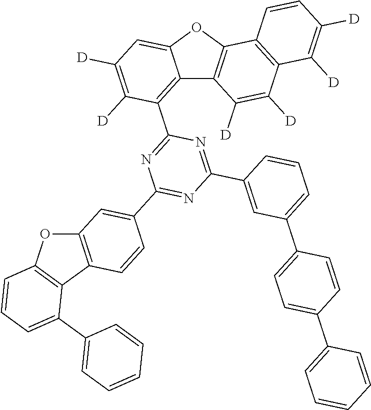 [SMILES: [2H]c1cc2oc3c4ccc([2H])c([2H])c4c([2H])c([2H])c3c2c(-c2nc(-c3cccc(-c4ccc(-c5ccccc5)cc4)c3)nc(-c3ccc4c(c3)oc3cccc(-c5ccccc5)c34)n2)c1[2H]] | 83 |
| 1- 305 | 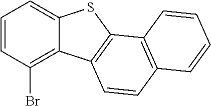 [SMILES: Brc1cccc2sc3c4ccccc4ccc3c12] | 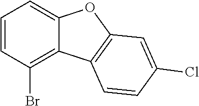 [SMILES: Clc1ccc2c(c1)oc1cccc(Br)c12] | 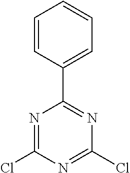 [SMILES: Clc1nc(Cl)nc(-c2ccccc2)n1] | 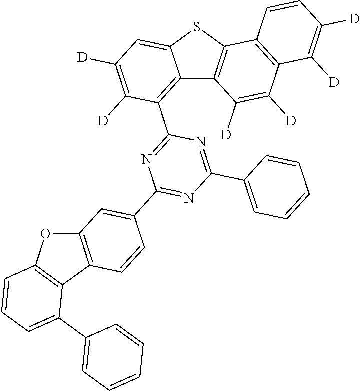 [SMILES: [2H]c1cc2sc3c4ccc([2H])c([2H])c4c([2H])c([2H])c3c2c(-c2nc(-c3ccccc3)nc(-c3ccc4c(c3)oc3cccc(-c5ccccc5)c34)n2)c1[2H]] | 82 |
| 1- 306 | 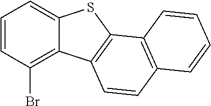 [SMILES: Brc1cccc2sc3c4ccccc4ccc3c12] | 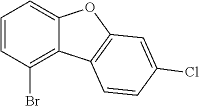 [SMILES: Clc1ccc2c(c1)oc1cccc(Br)c12] | 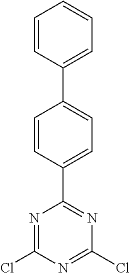 [SMILES: Clc1nc(Cl)nc(-c2ccc(-c3ccccc3)cc2)n1] | 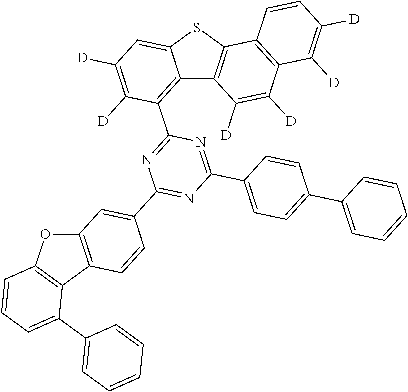 [SMILES: [2H]c1cc2sc3c4ccc([2H])c([2H])c4c([2H])c([2H])c3c2c(-c2nc(-c3ccc(-c4ccccc4)cc3)nc(-c3ccc4c(c3)oc3cccc(-c5ccccc5)c34)n2)c1[2H]] | 83 |
| 1- 307 | 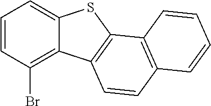 [SMILES: Brc1cccc2sc3c4ccccc4ccc3c12] | 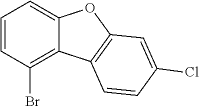 [SMILES: Clc1ccc2c(c1)oc1cccc(Br)c12] | 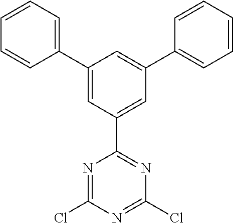 [SMILES: Clc1nc(Cl)nc(-c2cc(-c3ccccc3)cc(-c3ccccc3)c2)n1] | 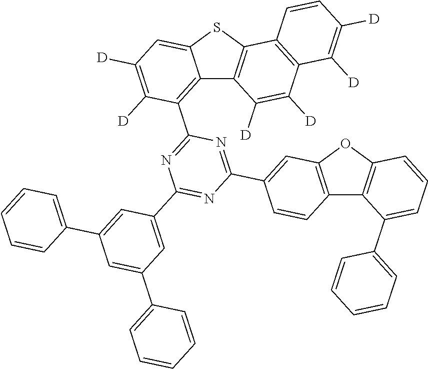 [SMILES: [2H]c1cc2sc3c4ccc([2H])c([2H])c4c([2H])c([2H])c3c2c(-c2nc(-c3cc(-c4ccccc4)cc(-c4ccccc4)c3)nc(-c3ccc4c(c3)oc3cccc(-c5ccccc5)c34)n2)c1[2H]] | 84 |
| 1- 308 | 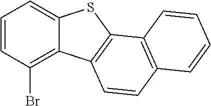 [SMILES: Brc1cccc2sc3c4ccccc4ccc3c12] | 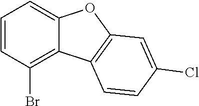 [SMILES: Clc1ccc2c(c1)oc1cccc(Br)c12] | 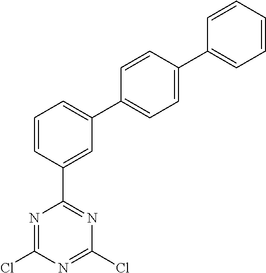 [SMILES: Clc1nc(Cl)nc(-c2cccc(-c3ccc(-c4ccccc4)cc3)c2)n1] | 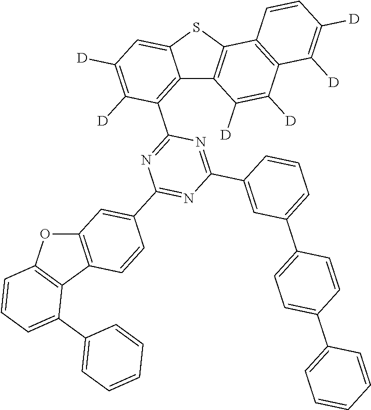 [SMILES: [2H]c1cc2sc3c4ccc([2H])c([2H])c4c([2H])c([2H])c3c2c(-c2nc(-c3cccc(-c4ccc(-c5ccccc5)cc4)c3)nc(-c3ccc4c(c3)oc3cccc(-c5ccccc5)c34)n2)c1[2H]] | 80 |

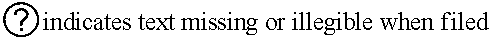

### [Preparation Example 5] Preparation of Compound 1-289

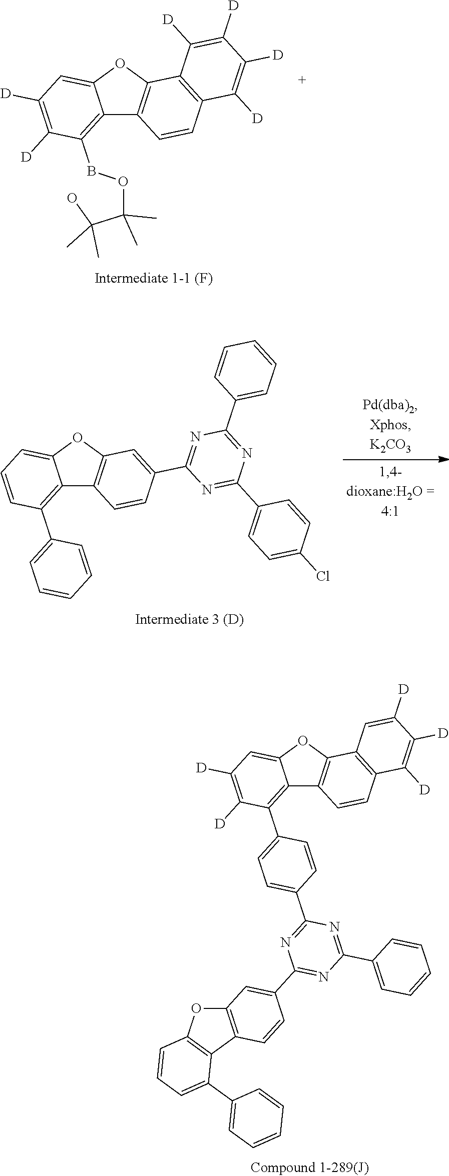

In Preparation Example 5, the synthesis of Intermediate 1-1(F) of Preparation Example 3 and Intermediate 3(D) of Preparation Example 2 was performed in the same manner as in the above-described preparation examples.

In Preparation Example 5, desired Compound (J) below was synthesized in the same manner as described above, except that Intermediate 1-1(F) and Intermediate 3(D) in Table 5 below were used.

TABLE 5

| | | | | Yield |
|---|---|---|---|---|
| Compound | Intermediate 1-1(F) | Intermediate 3(D) | Compound J | (%) |
| 1-290 | 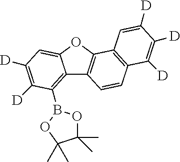 [SMILES: [2H]c1cc2c(ccc3c2oc2cc([2H])c([2H])c(B4OC(C)(C)C(C)(C)O4)c23)c([2H])c1[2H]] | 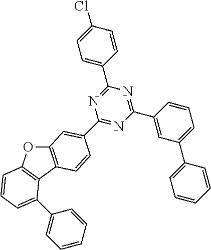 [SMILES: Clc1ccc(-c2nc(-c3cccc(-c4ccccc4)c3)nc(-c3ccc4c(c3)oc3cccc(-c5ccccc5)c34)n2)cc1] | 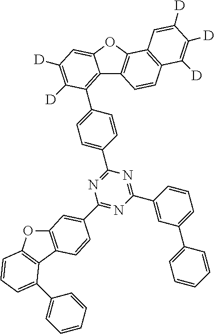 [SMILES: [2H]c1cc2c(ccc3c2oc2cc([2H])c([2H])c(-c4ccc(-c5nc(-c6cccc(-c7ccccc7)c6)nc(-c6ccc7c(c6)oc6cccc(-c8ccccc8)c67)n5)cc4)c23)c([2H])c1[2H]] | 73 |
| 1-291 | 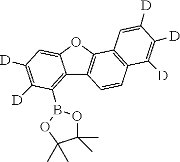 [SMILES: [2H]c1cc2c(ccc3c2oc2cc([2H])c([2H])c(B4OC(C)(C)C(C)(C)O4)c23)c([2H])c1[2H]] | 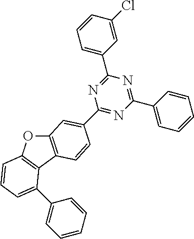 [SMILES: Clc1cccc(-c2nc(-c3ccccc3)nc(-c3ccc4c(c3)oc3cccc(-c5ccccc5)c34)n2)c1] | 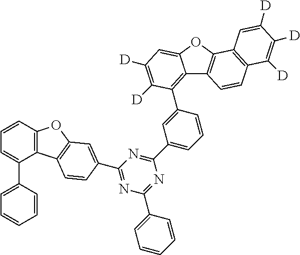 [SMILES: [2H]c1cc2c(ccc3c2oc2cc([2H])c([2H])c(-c4cccc(-c5nc(-c6ccccc6)nc(-c6ccc7c(c6)oc6cccc(-c8ccccc8)c67)n5)c4)c23)c([2H])c1[2H]] | 84 |
| 1-292 | 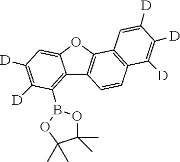 [SMILES: [2H]c1cc2c(ccc3c2oc2cc([2H])c([2H])c(B4OC(C)(C)C(C)(C)O4)c23)c([2H])c1[2H]] | 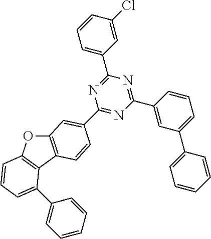 [SMILES: Clc1cccc(-c2nc(-c3cccc(-c4ccccc4)c3)nc(-c3ccc4c(c3)oc3cccc(-c5ccccc5)c34)n2)c1] | 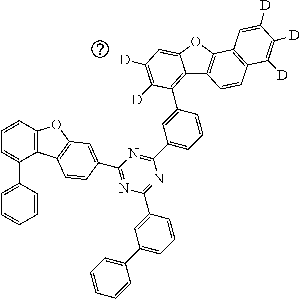 [SMILES: [2H]c1cc2c(ccc3c2oc2cc([2H])c([2H])c(-c4cccc(-c5nc(-c6cccc(-c7ccccc7)c6)nc(-c6ccc7c(c6)oc6cccc(-c8ccccc8)c67)n5)c4)c23)c([2H])c1[2H]] | 83 |
| 1-309 | 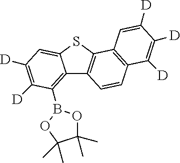 [SMILES: [2H]c1cc2c(ccc3c2sc2cc([2H])c([2H])c(B4OC(C)(C)C(C)(C)O4)c23)c([2H])c1[2H]] | 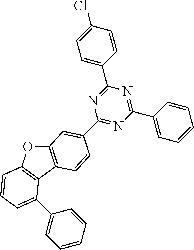 [SMILES: Clc1ccc(-c2nc(-c3ccccc3)nc(-c3ccc4c(c3)oc3cccc(-c5ccccc5)c34)n2)cc1] | 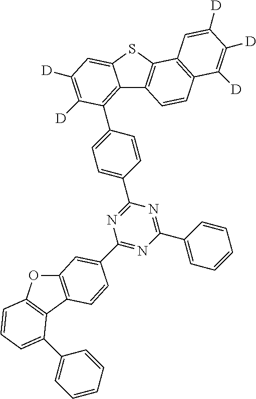 [SMILES: [2H]c1cc2c(ccc3c2sc2cc([2H])c([2H])c(-c4ccc(-c5nc(-c6ccccc6)nc(-c6ccc7c(c6)oc6cccc(-c8ccccc8)c67)n5)cc4)c23)c([2H])c1[2H]] | 82 |
| 1-310 | 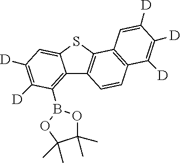 [SMILES: [2H]c1cc2c(ccc3c2sc2cc([2H])c([2H])c(B4OC(C)(C)C(C)(C)O4)c23)c([2H])c1[2H]] | 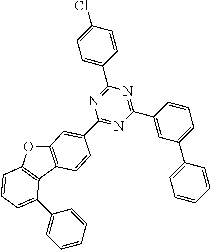 [SMILES: Clc1ccc(-c2nc(-c3cccc(-c4ccccc4)c3)nc(-c3ccc4c(c3)oc3cccc(-c5ccccc5)c34)n2)cc1] | 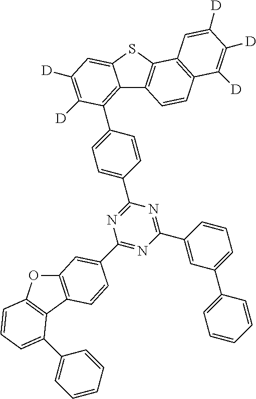 [SMILES: [2H]c1cc2c(ccc3c2sc2cc([2H])c([2H])c(-c4ccc(-c5nc(-c6cccc(-c7ccccc7)c6)nc(-c6ccc7c(c6)oc6cccc(-c8ccccc8)c67)n5)cc4)c23)c([2H])c1[2H]] | 83 |
| 1-311 | 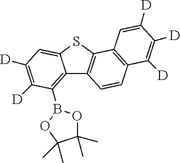 [SMILES: [2H]c1cc2c(ccc3c2sc2cc([2H])c([2H])c(B4OC(C)(C)C(C)(C)O4)c23)c([2H])c1[2H]] | 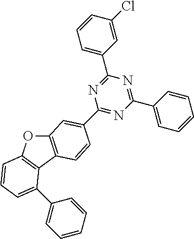 [SMILES: Clc1cccc(-c2nc(-c3ccccc3)nc(-c3ccc4c(c3)oc3cccc(-c5ccccc5)c34)n2)c1] | 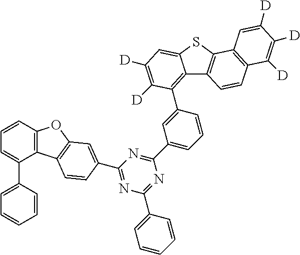 [SMILES: [2H]c1cc2c(ccc3c2sc2cc([2H])c([2H])c(-c4cccc(-c5nc(-c6ccccc6)nc(-c6ccc7c(c6)oc6cccc(-c8ccccc8)c67)n5)c4)c23)c([2H])c1[2H]] | 84 |
| 1-312 | 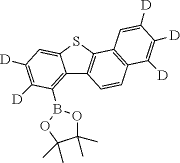 [SMILES: [2H]c1cc2c(ccc3c2sc2cc([2H])c([2H])c(B4OC(C)(C)C(C)(C)O4)c23)c([2H])c1[2H]] | 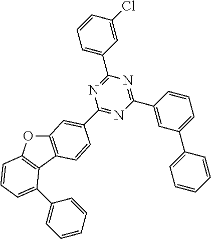 [SMILES: Clc1cccc(-c2nc(-c3cccc(-c4ccccc4)c3)nc(-c3ccc4c(c3)oc3cccc(-c5ccccc5)c34)n2)c1] | 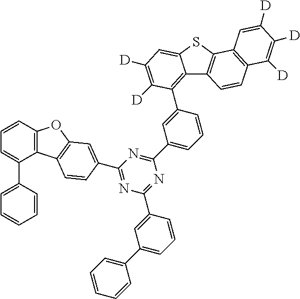 [SMILES: [2H]c1cc2c(ccc3c2sc2cc([2H])c([2H])c(-c4cccc(-c5nc(-c6cccc(-c7ccccc7)c6)nc(-c6ccc7c(c6)oc6cccc(-c8ccccc8)c67)n5)c4)c23)c([2H])c1[2H]] | 80 |

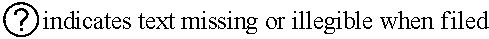

### [Preparation Example 6] Preparation of Compound 1-293

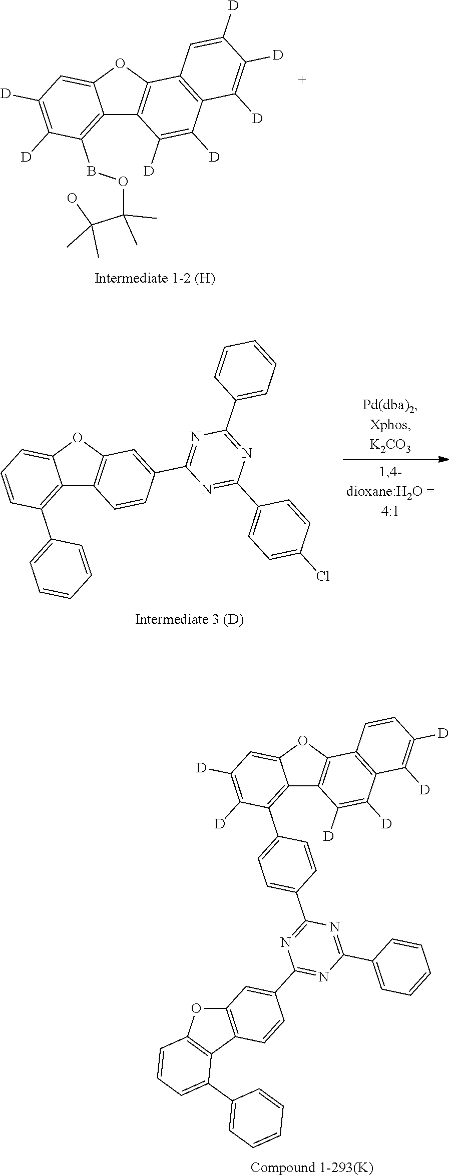

In Preparation Example 6, the synthesis of Intermediate 1-2(H) of Preparation Example 4 and Intermediate 3(D) of Preparation Example 2 were performed in the same manner as in the above-described Preparation Examples.

In Preparation Example 6, desired Compound (K) below was synthesized in the same manner as described above, except that Intermediates 1-2(H) and 3(D) in Table 6 below were used.

TABLE 6

| | | | | Yield |
|---|---|---|---|---|
| Compound | Intermediate 1-2(H) | Intermediate 3(D) | Compound K | (%) |
| 1-294 | 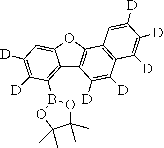 [SMILES: [2H]c1cc2c(c([2H])c1[2H])c([2H])c([2H])c1c2oc2cc([2H])c([2H])c(B3OC(C)(C)C(C)(C)O3)c21] | 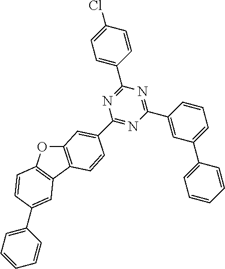 [SMILES: Clc1ccc(-c2nc(-c3cccc(-c4ccccc4)c3)nc(-c3ccc4c(c3)oc3ccc(-c5ccccc5)cc34)n2)cc1] | 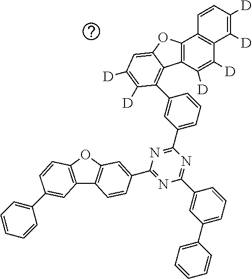 [SMILES: [2H]c1cc2oc3c4ccc([2H])c([2H])c4c([2H])c([2H])c3c2c(-c2cccc(-c3nc(-c4cccc(-c5ccccc5)c4)nc(-c4ccc5c(c4)oc4ccc(-c6ccccc6)cc45)n3)c2)c1[2H]] | 70 |
| 1-295 | 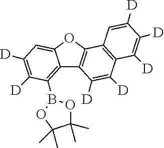 [SMILES: [2H]c1cc2c(c([2H])c1[2H])c([2H])c([2H])c1c2oc2cc([2H])c([2H])c(B3OC(C)(C)C(C)(C)O3)c21] | 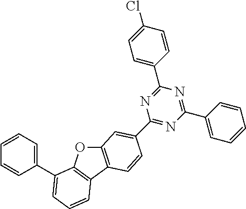 [SMILES: Clc1ccc(-c2nc(-c3ccccc3)nc(-c3ccc4c(c3)oc3c(-c5ccccc5)cccc34)n2)cc1] | 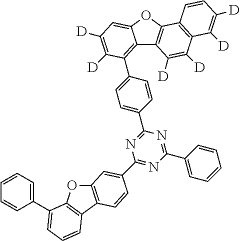 [SMILES: [2H]c1cc2oc3c4ccc([2H])c([2H])c4c([2H])c([2H])c3c2c(-c2ccc(-c3nc(-c4ccccc4)nc(-c4ccc5c(c4)oc4c(-c6ccccc6)cccc45)n3)cc2)c1[2H]] | 74 |
| 1-296 | 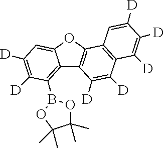 [SMILES: [2H]c1cc2c(c([2H])c1[2H])c([2H])c([2H])c1c2oc2cc([2H])c([2H])c(B3OC(C)(C)C(C)(C)O3)c21] | 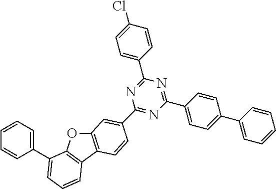 [SMILES: Clc1ccc(-c2nc(-c3ccc(-c4ccccc4)cc3)nc(-c3ccc4c(c3)oc3c(-c5ccccc5)cccc34)n2)cc1] | 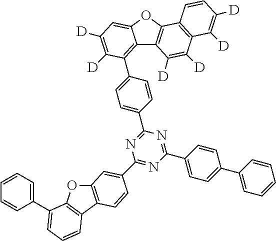 [SMILES: [2H]c1cc2oc3c4ccc([2H])c([2H])c4c([2H])c([2H])c3c2c(-c2ccc(-c3nc(-c4ccc(-c5ccccc5)cc4)nc(-c4ccc5c(c4)oc4c(-c6ccccc6)cccc45)n3)cc2)c1[2H]] | 73 |
| 1-313 | 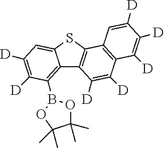 [SMILES: [2H]c1cc2c(c([2H])c1[2H])c([2H])c([2H])c1c2sc2cc([2H])c([2H])c(B3OC(C)(C)C(C)(C)O3)c21] | 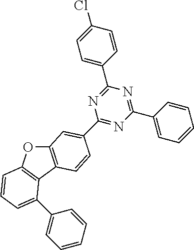 [SMILES: Clc1ccc(-c2nc(-c3ccccc3)nc(-c3ccc4c(c3)oc3cccc(-c5ccccc5)c34)n2)cc1] | 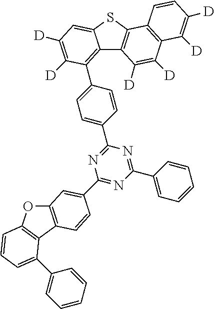 [SMILES: [2H]c1cc2sc3c4ccc([2H])c([2H])c4c([2H])c([2H])c3c2c(-c2ccc(-c3nc(-c4ccccc4)nc(-c4ccc5c(c4)oc4cccc(-c6ccccc6)c45)n3)cc2)c1[2H]] | 72 |
| 1-314 | 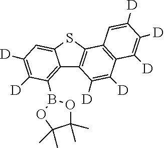 [SMILES: [2H]c1cc2c(c([2H])c1[2H])c([2H])c([2H])c1c2sc2cc([2H])c([2H])c(B3OC(C)(C)C(C)(C)O3)c21] | 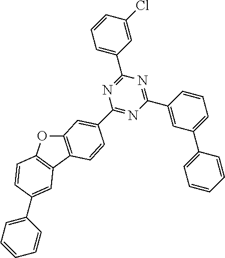 [SMILES: Clc1cccc(-c2nc(-c3cccc(-c4ccccc4)c3)nc(-c3ccc4c(c3)oc3ccc(-c5ccccc5)cc34)n2)c1] | 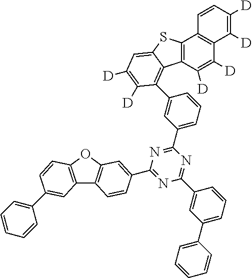 [SMILES: [2H]c1cc2sc3c4ccc([2H])c([2H])c4c([2H])c([2H])c3c2c(-c2cccc(-c3nc(-c4cccc(-c5ccccc5)c4)nc(-c4ccc5c(c4)oc4ccc(-c6ccccc6)cc45)n3)c2)c1[2H]] | 83 |
| 1-315 | 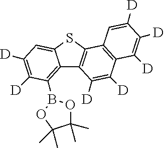 [SMILES: [2H]c1cc2c(c([2H])c1[2H])c([2H])c([2H])c1c2sc2cc([2H])c([2H])c(B3OC(C)(C)C(C)(C)O3)c21] | 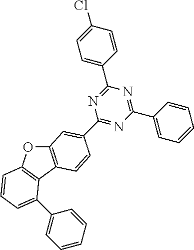 [SMILES: Clc1ccc(-c2nc(-c3ccccc3)nc(-c3ccc4c(c3)oc3cccc(-c5ccccc5)c34)n2)cc1] | 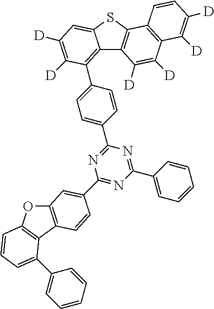 [SMILES: [2H]c1cc2sc3c4ccc([2H])c([2H])c4c([2H])c([2H])c3c2c(-c2ccc(-c3nc(-c4ccccc4)nc(-c4ccc5c(c4)oc4cccc(-c6ccccc6)c45)n3)cc2)c1[2H]] | 84 |
| 1-316 | 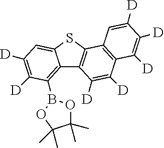 [SMILES: [2H]c1cc2c(c([2H])c1[2H])c([2H])c([2H])c1c2sc2cc([2H])c([2H])c(B3OC(C)(C)C(C)(C)O3)c21] | 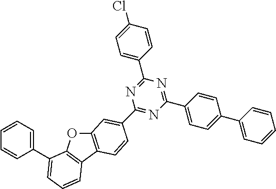 [SMILES: Clc1ccc(-c2nc(-c3ccc(-c4ccccc4)cc3)nc(-c3ccc4c(c3)oc3c(-c5ccccc5)cccc34)n2)cc1] | 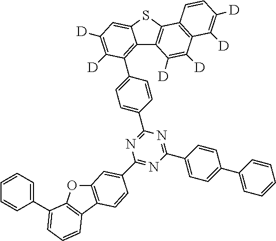 [SMILES: [2H]c1cc2sc3c4ccc([2H])c([2H])c4c([2H])c([2H])c3c2c(-c2ccc(-c3nc(-c4ccc(-c5ccccc5)cc4)nc(-c4ccc5c(c4)oc4c(-c6ccccc6)cccc45)n3)cc2)c1[2H]] | 80 |

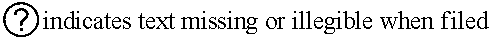

### [Preparation Example 7] Preparation of Compound 1-297

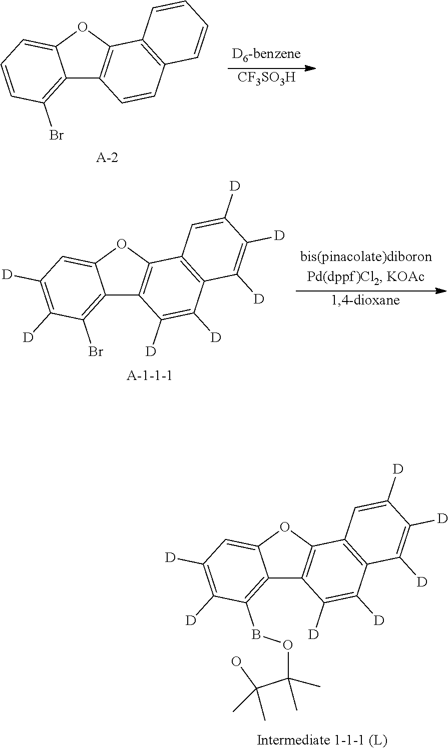

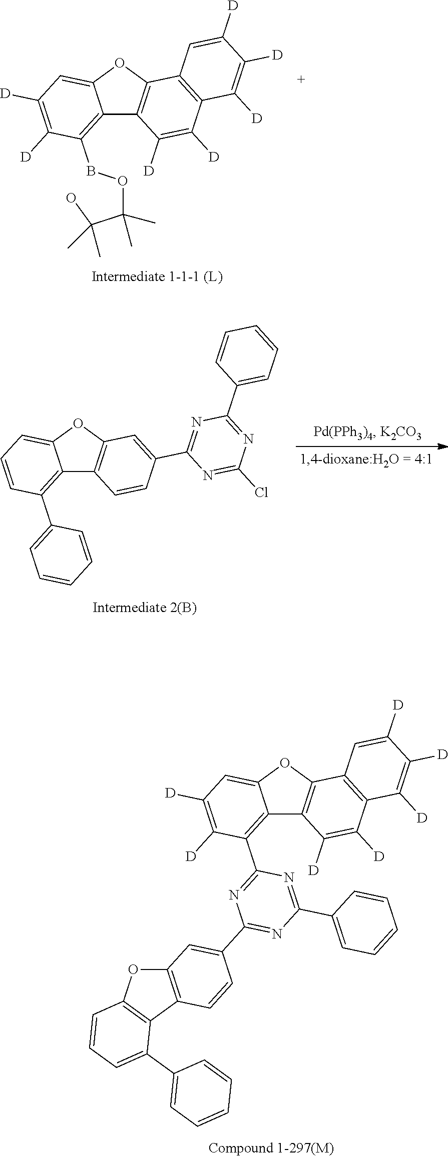

### Synthesis of A-1-1-1

7-bromonaphtho[1,2-b]benzofuran) (A-2)(10 g, 1 eq.), D6-benzene (500 g, 5941.7 mmol.), and CF₃SO₃H (255 g, 1613 mmol) were put into a reaction flask, and reacted at 40° C. for 3 hours. After completing the reaction, H₂O was added for neutralization, MC and H₂O were added for extraction, and an organic layer was purified using a column, thereby obtaining Compound A-1-1-1 (6.5 g, 65%).

In Preparation Example 7, desired Compound (M) was synthesized in the same manner as described above, except that Reagents A-2, B-4 and B-1 of Table 7 below were used.

TABLE 7

| | | | | | Yield |
|---|---|---|---|---|---|
| Compound | A-2 | B-4 | B-1 | CompoundM | (%) |
| 1-298 | 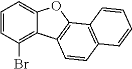 [SMILES: Brc1cccc2oc3c4ccccc4ccc3c12] | 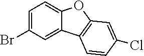 [SMILES: Clc1ccc2c(c1)oc1ccc(Br)cc12] | 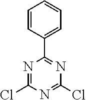 [SMILES: Clc1nc(Cl)nc(-c2ccccc2)n1] | 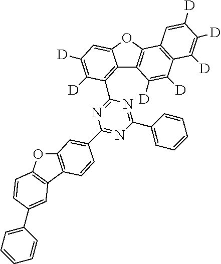 [SMILES: [2H]c1cc2c(c([2H])c1[2H])c([2H])c([2H])c1c2oc2cc([2H])c([2H])c(-c3nc(-c4ccccc4)nc(-c4ccc5c(c4)oc4ccc(-c6ccccc6)cc45)n3)c21] | 83 |
| 1-299 | 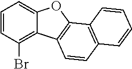 [SMILES: Brc1cccc2oc3c4ccccc4ccc3c12] | 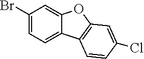 [SMILES: Clc1ccc2c(c1)oc1cc(Br)ccc12] | 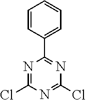 [SMILES: Clc1nc(Cl)nc(-c2ccccc2)n1] | 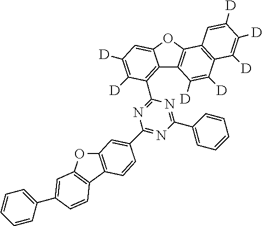 [SMILES: [2H]c1cc2c(c([2H])c1[2H])c([2H])c([2H])c1c2oc2cc([2H])c([2H])c(-c3nc(-c4ccccc4)nc(-c4ccc5c(c4)oc4cc(-c6ccccc6)ccc45)n3)c21] | 84 |
| 1-300 | 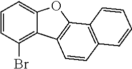 [SMILES: Brc1cccc2oc3c4ccccc4ccc3c12] | 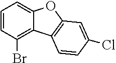 [SMILES: Clc1ccc2c(c1)oc1cccc(Br)c12] | 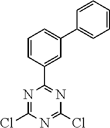 [SMILES: Clc1nc(Cl)nc(-c2cccc(-c3ccccc3)c2)n1] | 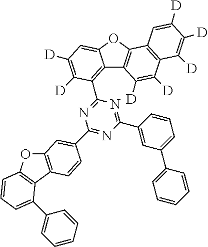 [SMILES: [2H]c1cc2c(c([2H])c1[2H])c([2H])c([2H])c1c2oc2cc([2H])c([2H])c(-c3nc(-c4cccc(-c5ccccc5)c4)nc(-c4ccc5c(c4)oc4cccc(-c6ccccc6)c45)n3)c21] | 83 |
| 1-317 | 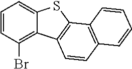 [SMILES: Brc1cccc2sc3c4ccccc4ccc3c12] | 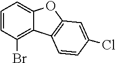 [SMILES: Clc1ccc2c(c1)oc1cccc(Br)c12] | 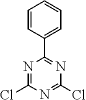 [SMILES: Clc1nc(Cl)nc(-c2ccccc2)n1] | 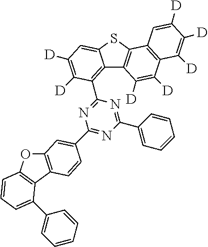 [SMILES: [2H]c1cc2c(c([2H])c1[2H])c([2H])c([2H])c1c2sc2cc([2H])c([2H])c(-c3nc(-c4ccccc4)nc(-c4ccc5c(c4)oc4cccc(-c6ccccc6)c45)n3)c21] | 82 |
| 1-318 | 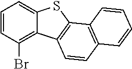 [SMILES: Brc1cccc2sc3c4ccccc4ccc3c12] | 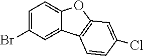 [SMILES: Clc1ccc2c(c1)oc1ccc(Br)cc12] | 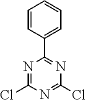 [SMILES: Clc1nc(Cl)nc(-c2ccccc2)n1] | 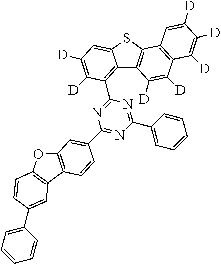 [SMILES: [2H]c1cc2c(c([2H])c1[2H])c([2H])c([2H])c1c2sc2cc([2H])c([2H])c(-c3nc(-c4ccccc4)nc(-c4ccc5c(c4)oc4ccc(-c6ccccc6)cc45)n3)c21] | 83 |
| 1-319 | 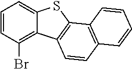 [SMILES: Brc1cccc2sc3c4ccccc4ccc3c12] | 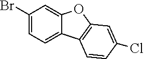 [SMILES: Clc1ccc2c(c1)oc1cc(Br)ccc12] | 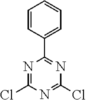 [SMILES: Clc1nc(Cl)nc(-c2ccccc2)n1] | 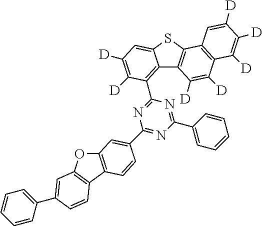 [SMILES: [2H]c1cc2c(c([2H])c1[2H])c([2H])c([2H])c1c2sc2cc([2H])c([2H])c(-c3nc(-c4ccccc4)nc(-c4ccc5c(c4)oc4cc(-c6ccccc6)ccc45)n3)c21] | 84 |
| 1-320 | 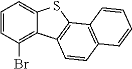 [SMILES: Brc1cccc2sc3c4ccccc4ccc3c12] | 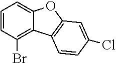 [SMILES: Clc1ccc2c(c1)oc1cccc(Br)c12] | 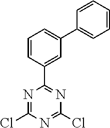 [SMILES: Clc1nc(Cl)nc(-c2cccc(-c3ccccc3)c2)n1] | 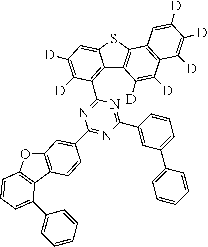 [SMILES: [2H]c1cc2c(c([2H])c1[2H])c([2H])c([2H])c1c2sc2cc([2H])c([2H])c(-c3nc(-c4cccc(-c5ccccc5)c4)nc(-c4ccc5c(c4)oc4cccc(-c6ccccc6)c45)n3)c21] | 80 |

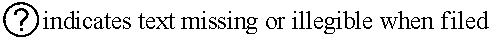

### [Preparation Example 8] Preparation of Compound 2-1(C)

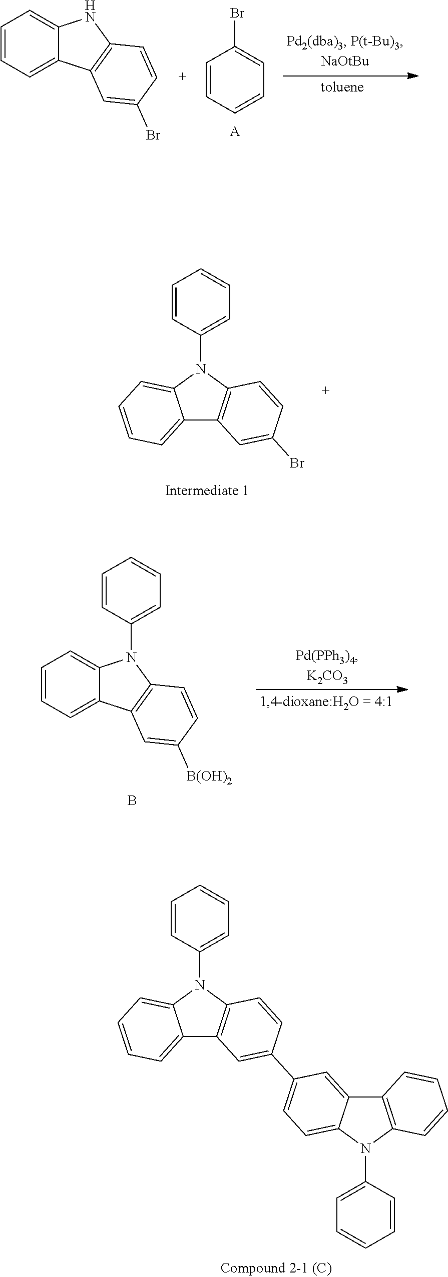

### Synthesis of Intermediate 1

3-bromo-9H-carbazole (10 g, 49.59 mmol), 2-bromobenzene-1-ylium (A) (24.2 g, 148.77 mmol), Pd₂ (dba)₃ (2.27 g, 2.48 mmol), P(t-BU)₃ (2.42 mL, 9.92 mmol), and NaOtBu (9.53 g, 99.18 mmol) were put into a reaction flask, toluene (100 mL) was added, and the reaction mixture was heated at 135° C. for 15 hours. After completing the reaction, MC and H₂O were added for extraction, and then column purification was performed, thereby obtaining Intermediate 1 (14 g, 98%).

### Method of Synthesizing Compound 2-1(C)

Intermediate 1 (14 g, 43.4 mmol), (9-phenyl-9H-carbazol-3-yl)boronic acid (B) (14.9 g, 52 mmol), Pd(PPh₃)₄ (2.5 g, 2.17 mmol), and K₂CO₃ (17.9 g, 130 mmol) and a 1,4-dioxane/water mixture (140 mL/35 mL) were then put into a reaction flask, and heated at 120° C. for 4 hours. After completing the reaction, a solid produced after lowering the temperature to room temperature was washed with distilled water and MeOH, thereby obtaining Compound 2-1(C) (17 g, 80%).

In Preparation Example 8, Compound (C) below was synthesized in the same manner as described above, except that Compounds A and B in Table 8 below were used.

TABLE 8

| | | | | Yield |
|---|---|---|---|---|
| Compound | A | B | C | (%) |
| 2-2 | 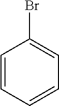 | 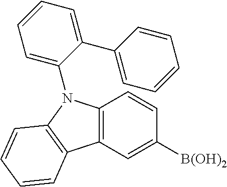 | 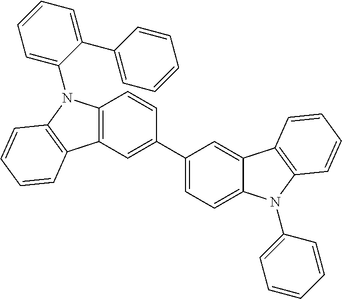 | 82 |
| 2-3 | 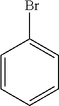 | 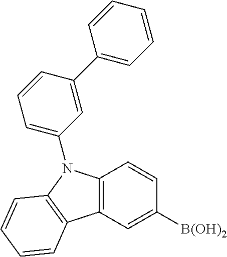 | 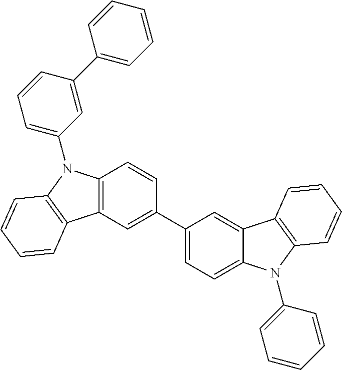 | 80 |
| 2-4 | 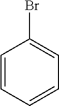 | 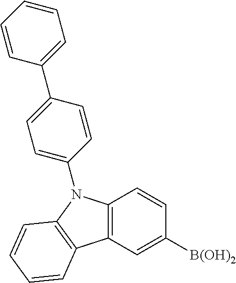 | 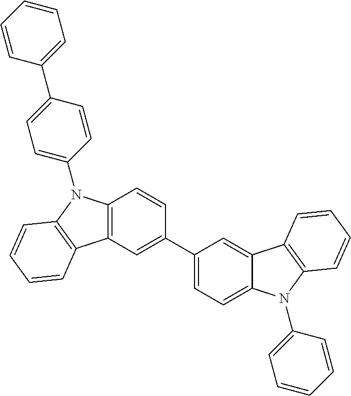 | 83 |
| 2-5 | 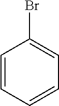 | 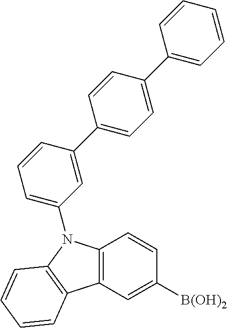 | 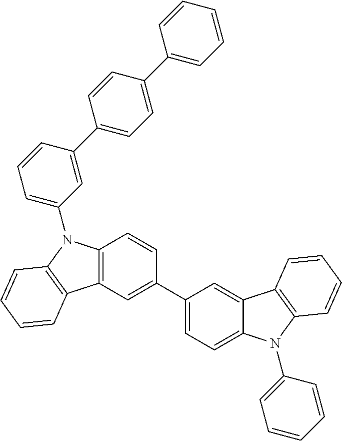 | 88 |
| 2-6 | 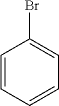 | 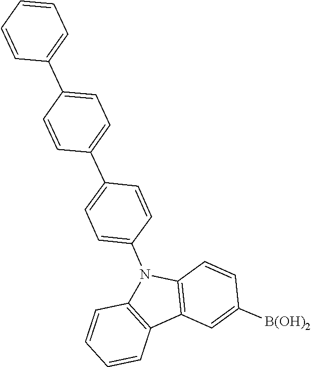 | 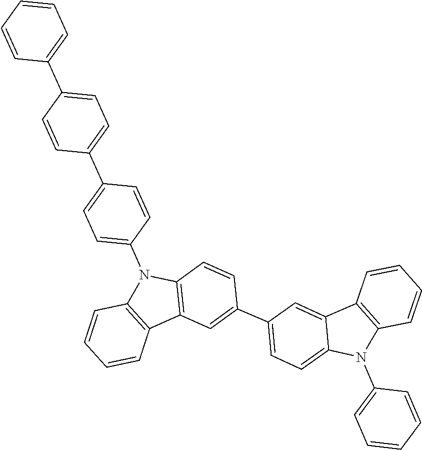 | 86 |
| 2-7 | 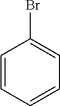 | 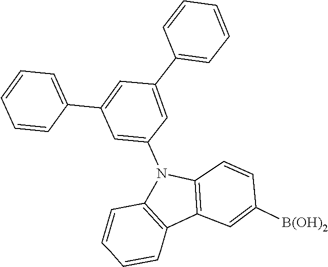 | 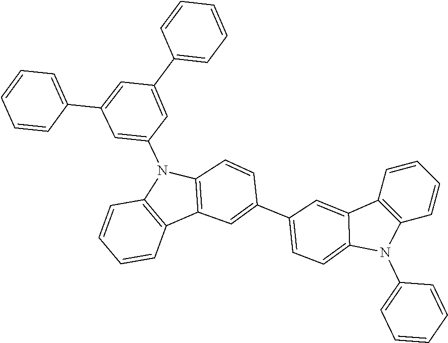 | 80 |
| 2-11 | 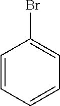 | 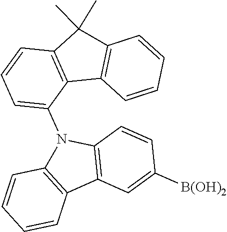 | 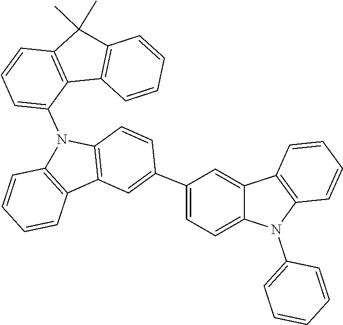 | 81 |
| 2-16 | 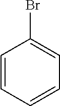 | 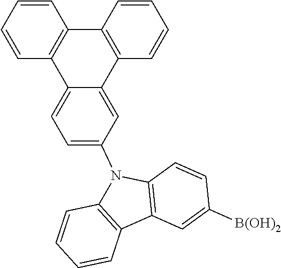 | 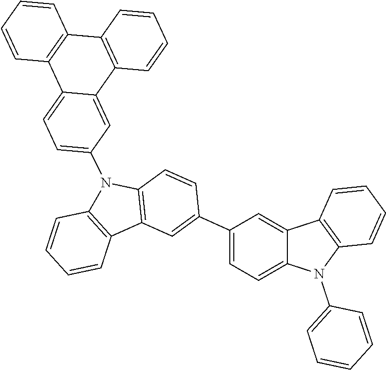 | 80 |
| 2-19 | 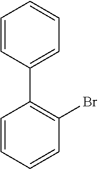 | 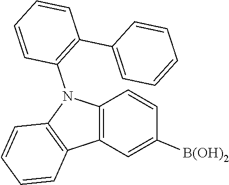 | 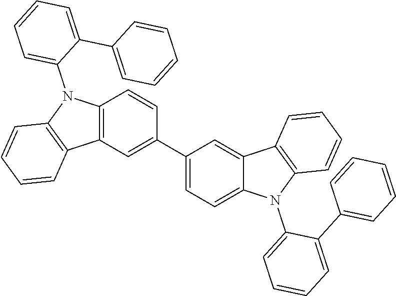 | 83 |
| 2-20 | 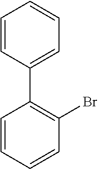 | 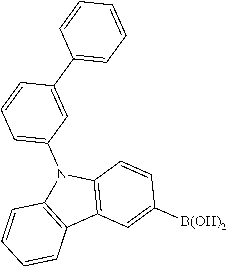 | 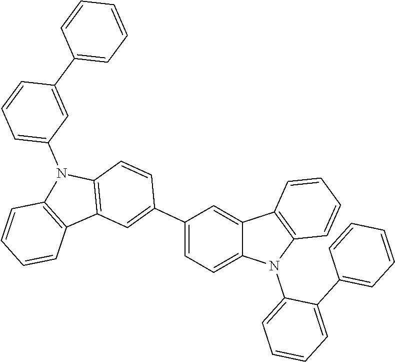 | 81 |
| 2-21 | 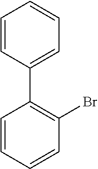 | 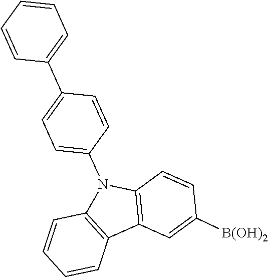 | 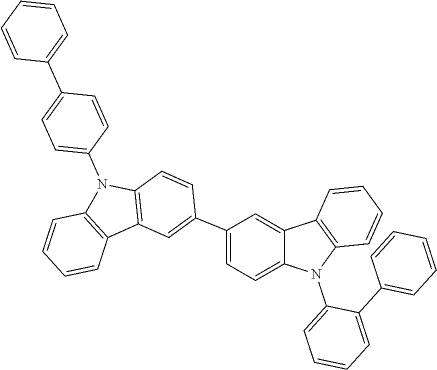 | 80 |
| 2-22 | 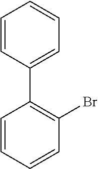 | 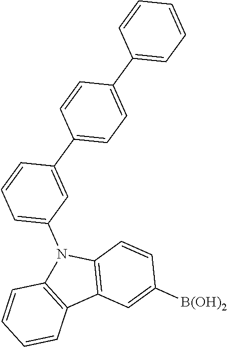 | 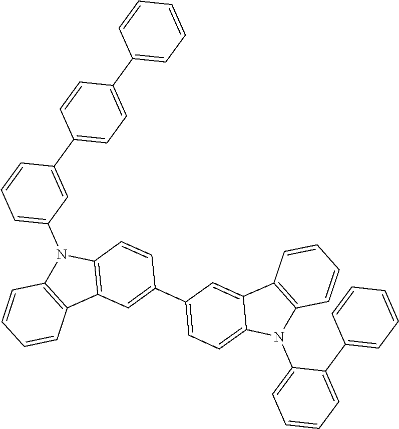 | 79 |
| 2-23 | 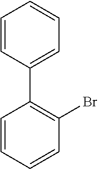 | 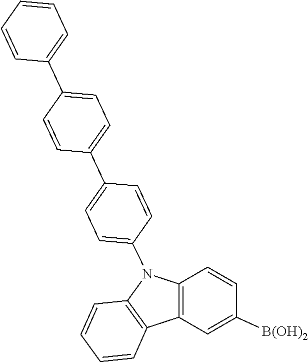 | 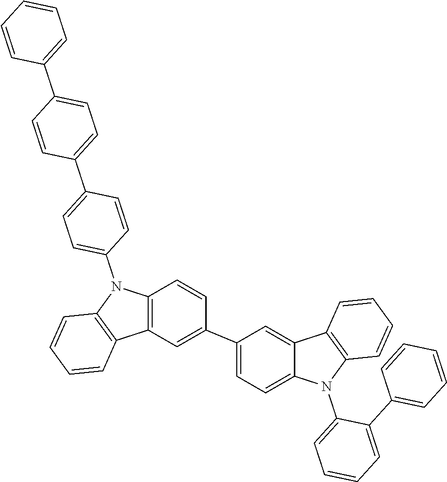 | 82 |
| 2-26 | 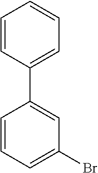 | 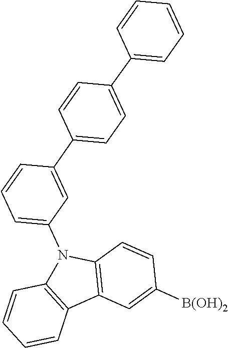 | 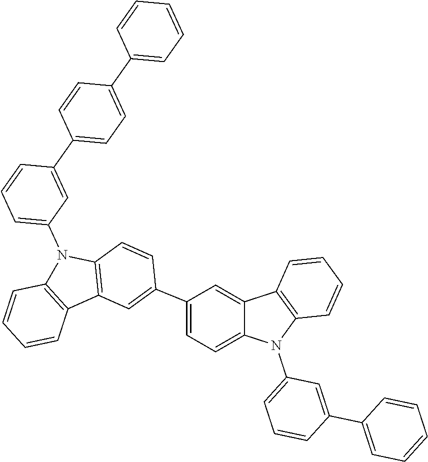 | 81 |
| 2-27 | 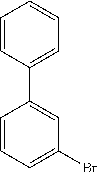 | 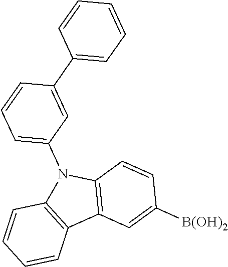 | 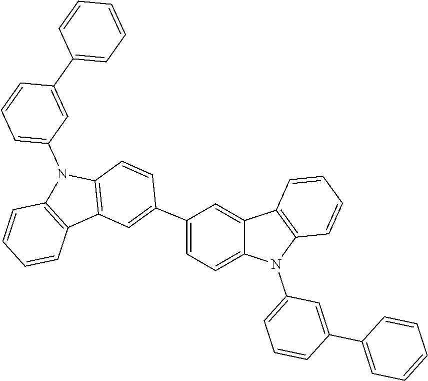 | 85 |
| 2-28 | 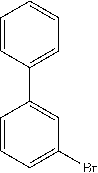 | 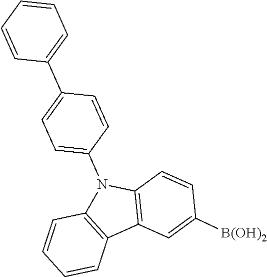 | 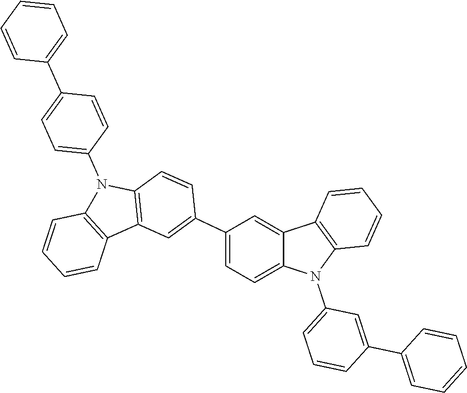 | 81 |
| 2-29 | 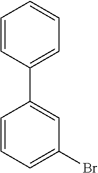 | 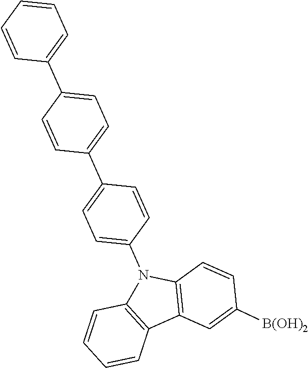 | 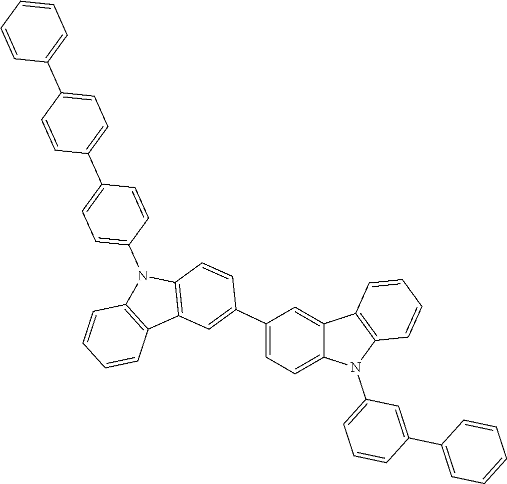 | 80 |
| 2-30 | 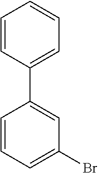 | 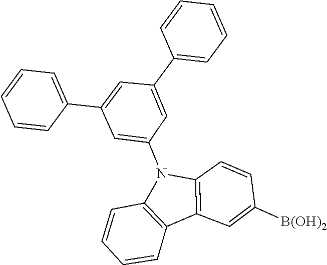 | 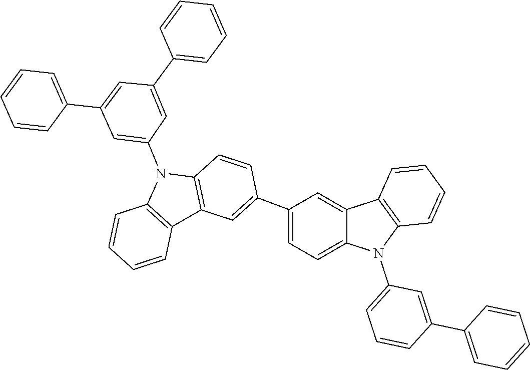 | 79 |
| 2-32 | 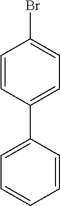 | 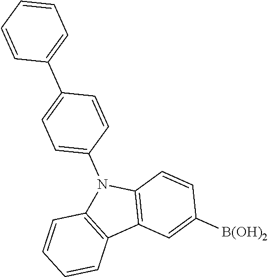 | 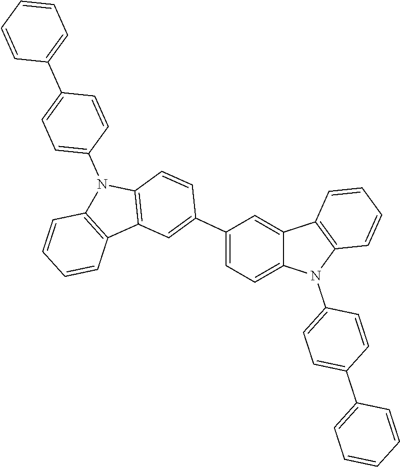 | 81 |
| 2-33 | 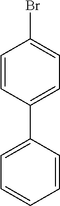 | 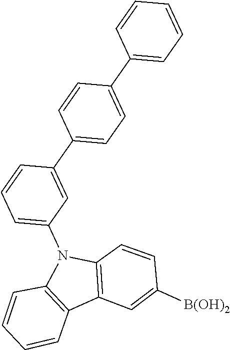 | 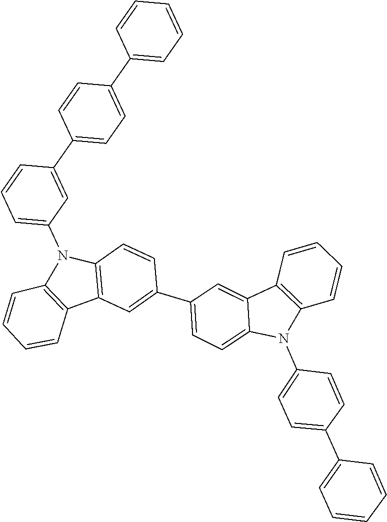 | 81 |
| 2-34 | 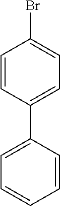 | 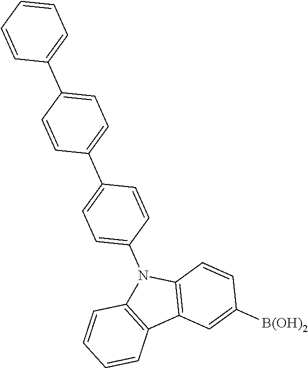 | 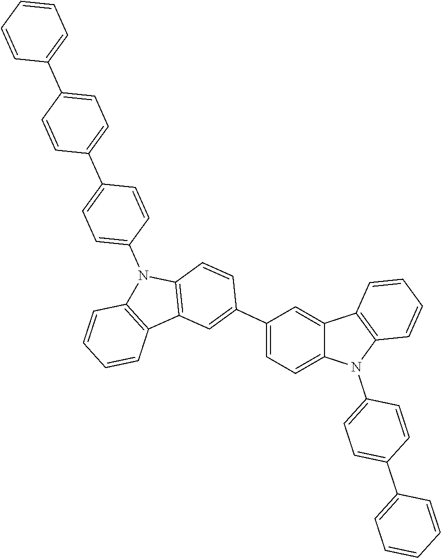 | 82 |
| 2-38 | 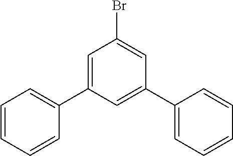 | 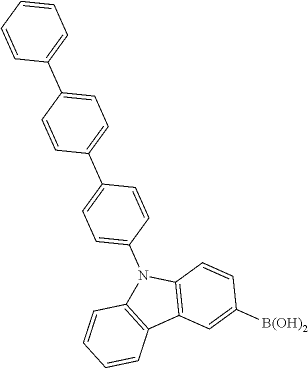 | 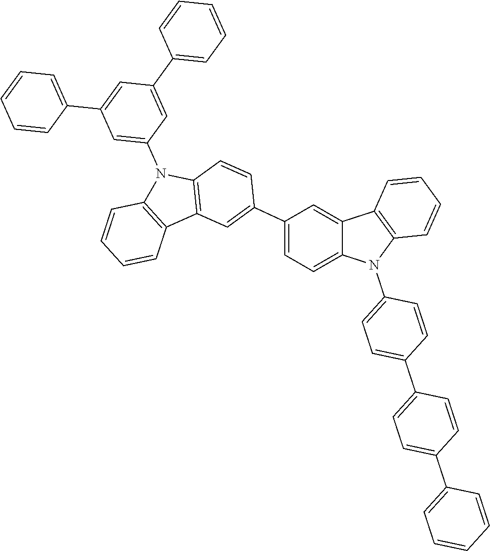 | 82 |
| 2-40 | 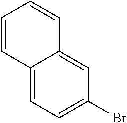 | 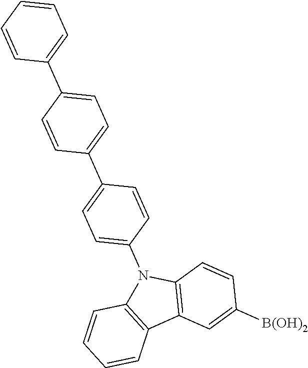 | 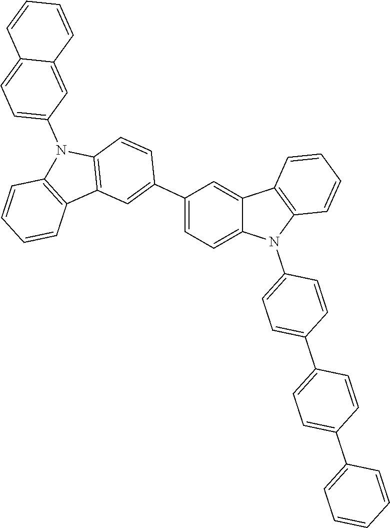 | 81 |
| 2-41 | 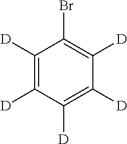 | 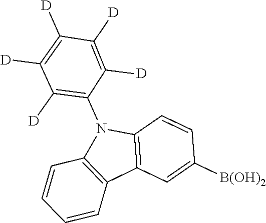 | 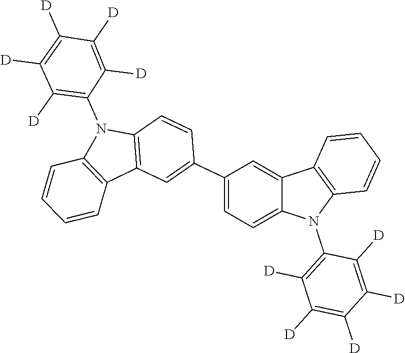 | 80 |
| 2-42 | 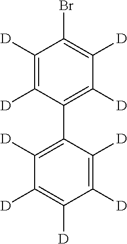 | 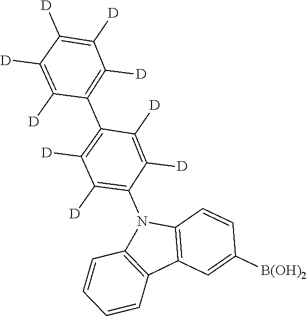 | 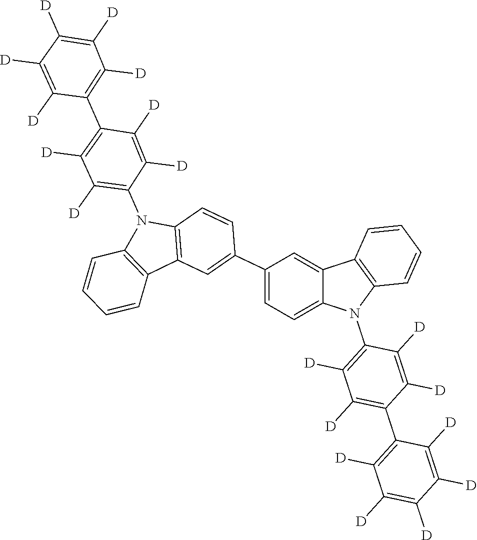 | 79 |
| 2-43 | 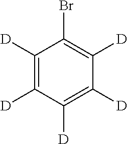 | 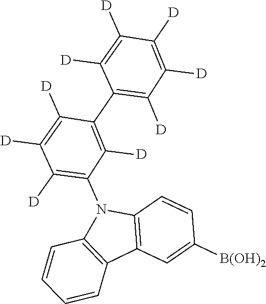 | 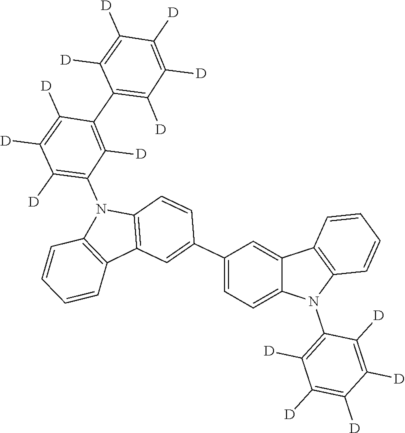 | 78 |
| 2-45 | 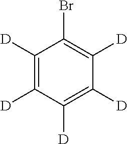 | 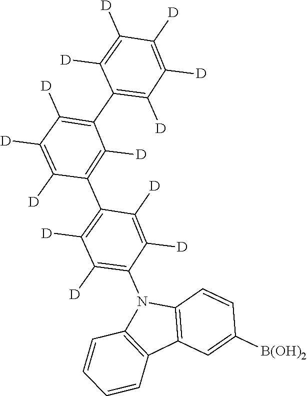 | 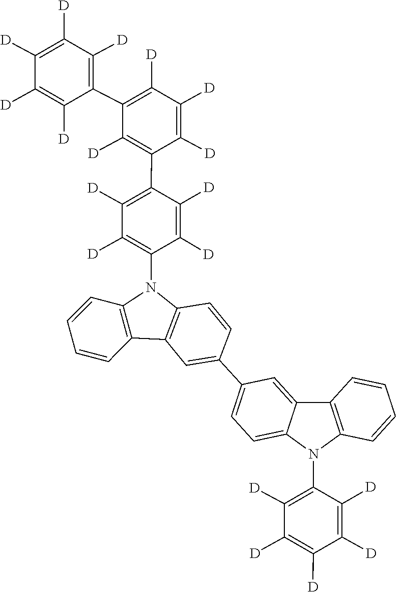 | 79 |
| 2-46 | 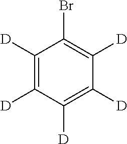 | 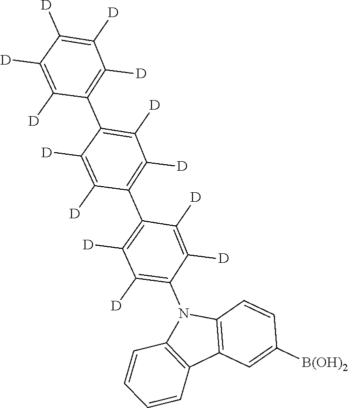 | 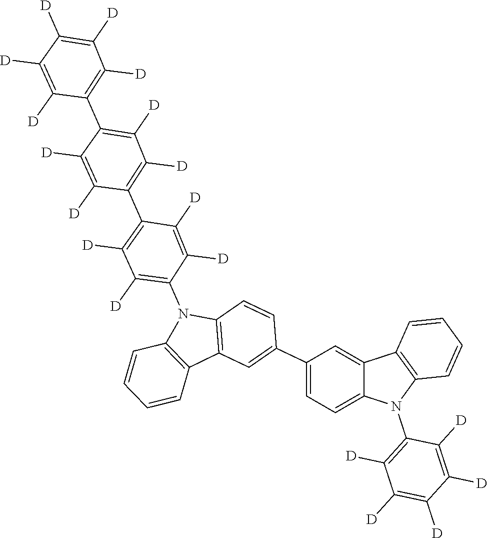 | 80 |
| 2-48 | 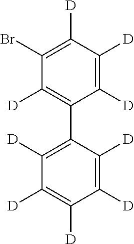 | 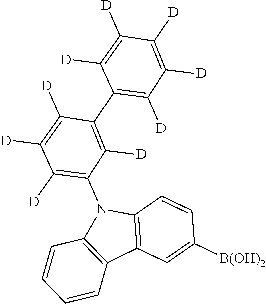 | 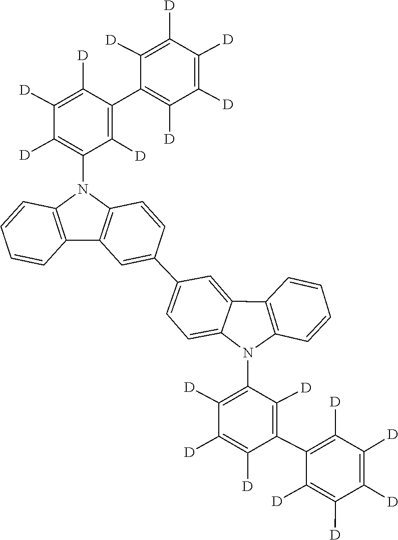 | 81 |
| 2-49 | 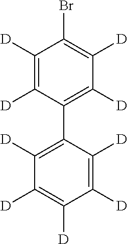 | 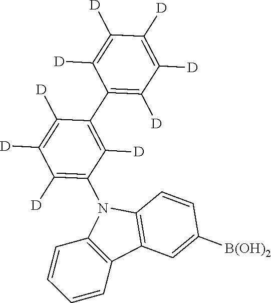 | 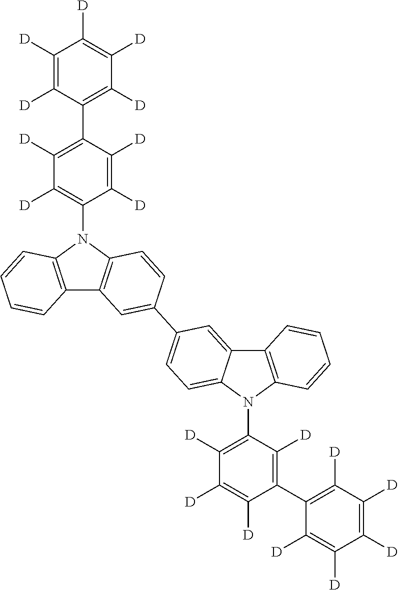 | 83 |
| 2-50 | 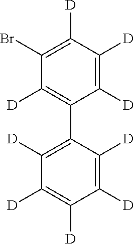 | 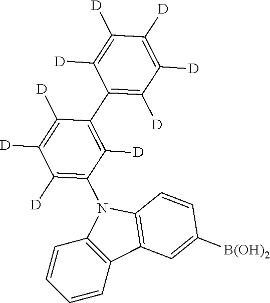 | 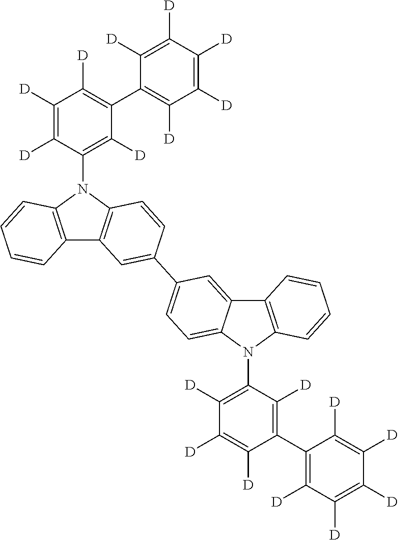 | 82 |
| 2-51 | 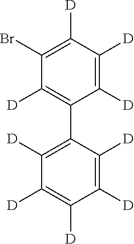 | 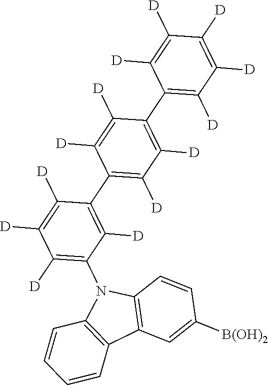 | 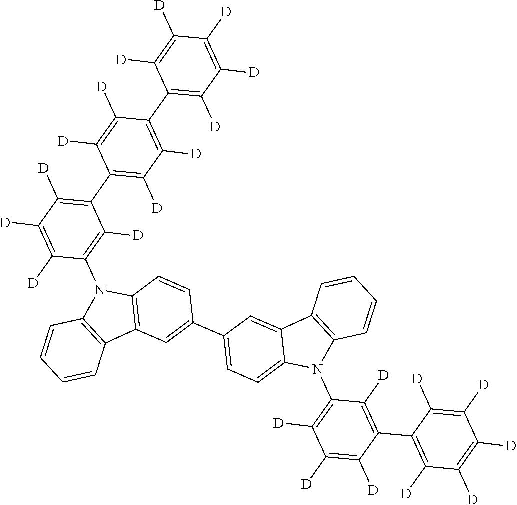 | 84 |
| 2-52 | 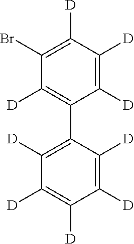 | 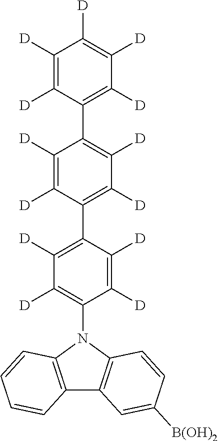 | 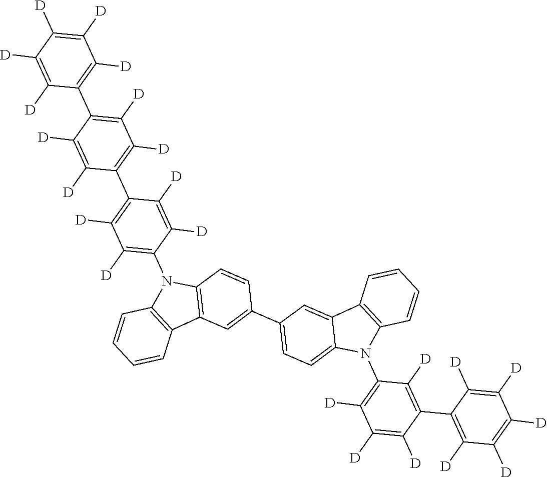 | 80 |
| 2-55 | 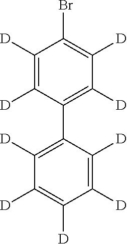 | 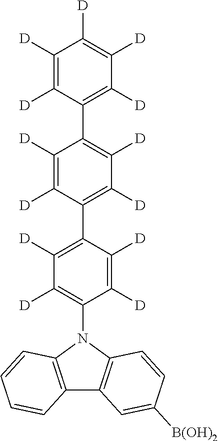 | 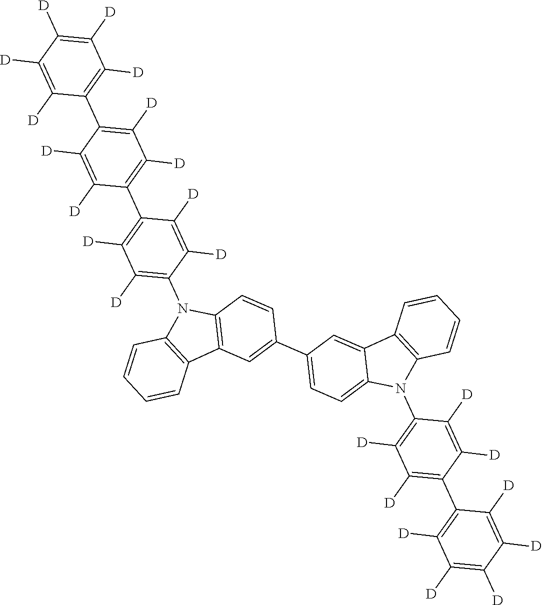 | 81 |
| 2-57 | 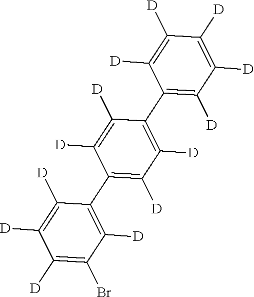 | 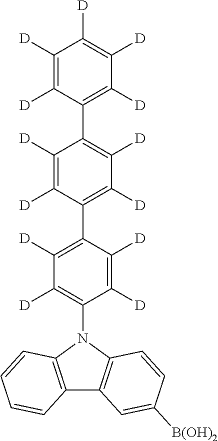 | 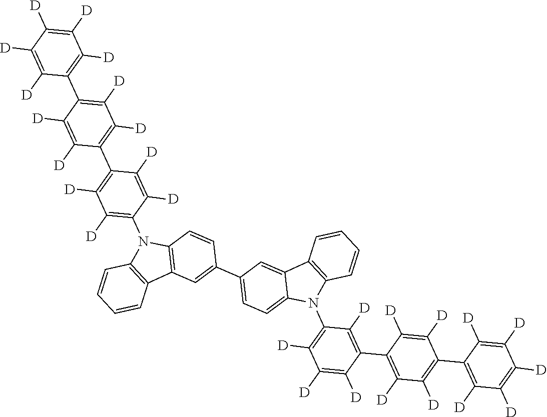 | 82 |
| 2-60 | 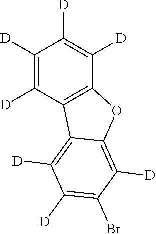 | 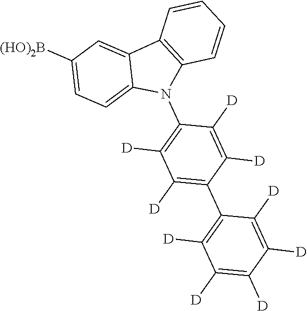 | 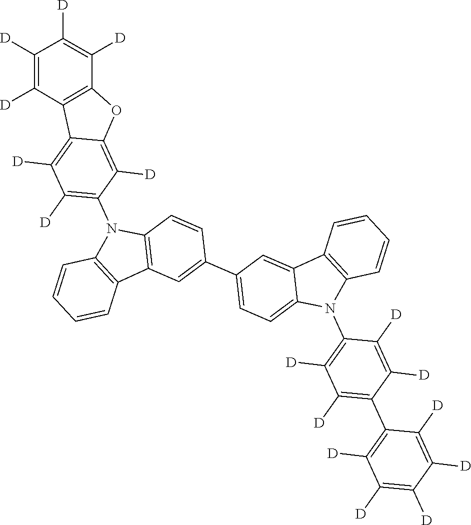 | 82 |

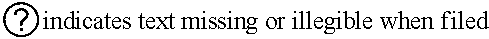

### [Preparation Example 9] Preparation of Compound 2-61(F)

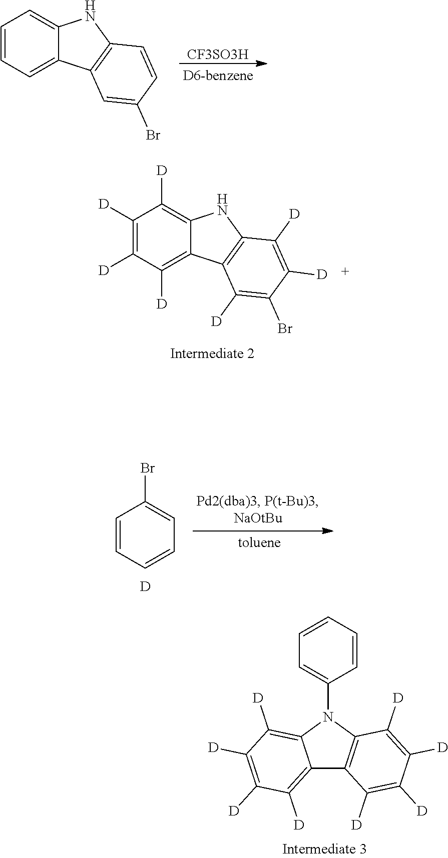

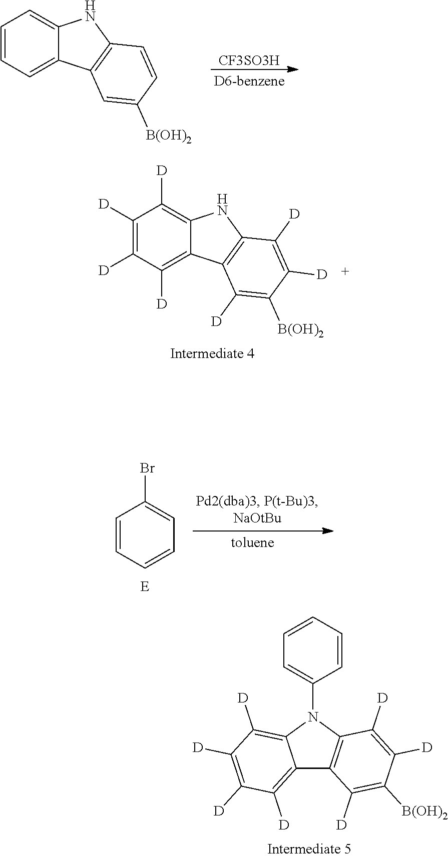

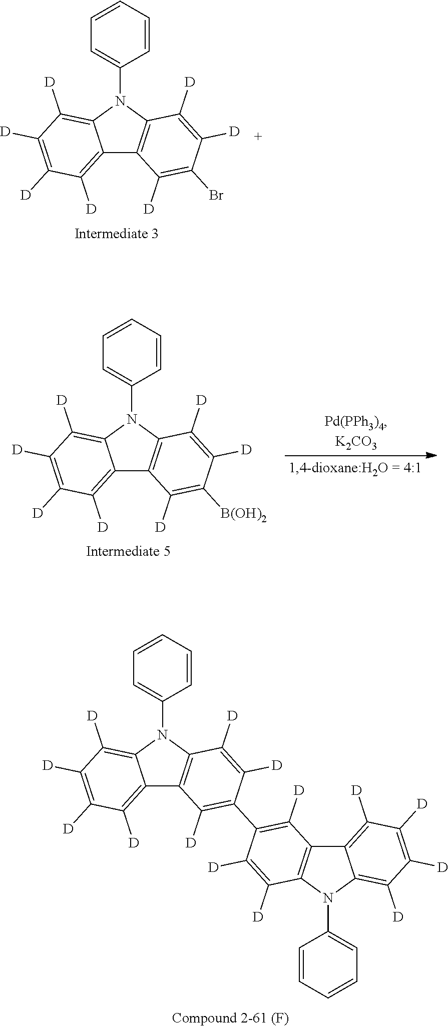 [SMILES: [2H]c1c([2H])c([2H])c2c(c1[2H])c1c([2H])c(Br)c([2H])c([2H])c1n2-c1ccccc1 ; [2H]c1c([2H])c([2H])c2c(c1[2H])c1c([2H])c(B(O)O)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

### Synthesis of Intermediate 2

3-bromo-9H-carbazole (10 g, 40.23 mmol) and 1,000 mL of D6-benzene were added, CF₃SO₃H (170 g, 1075 mmol) was added, and then the resulting mixture was stirred at 50° C. After completing the reaction, D20 was added for neutralization, MC and a Na₂CO₃ aqueous solution were added for extraction, and column purification was performed, thereby obtaining Intermediate 2 (10 g, 98%).

### Synthesis of Intermediate 3

Intermediate 2 (10 g, 39.5 mmol), bromobenzene (12.4 g, 79 mmol), Pd₂(dba)₃ (1.81 g, 1.98 mmol), P(t-Bu)₃ (1.93 mL, 7.9 mmol), and NaOtBu (11.4 g, 118.51 mmol) were added, toluene (100 mL) was added, and then the resulting mixture was heated at 135° C. for 10 hours. After completing the reaction, MC and H₂O were added for extraction, and column purification was performed, thereby obtaining Intermediate 3 (11 g, 84%).

### Synthesis of Intermediate 4

9H-carbazol-3-ylboronic acid (10 g, 47.3 mmol) and 1,000 mL of D6-benzene were added, CF₃SO₃H (170 g, 1,075 mmol) was added, and then the resulting mixture was stirred at 50° C. After completing the reaction, D20 was added for neutralization, MC and a Na₂CO₃ aqueous solution were used for extraction, and column purification was performed, thereby obtaining Intermediate 4 (9 g, 87%).

### Synthesis of Intermediate 5

Intermediate 4 (9 g, 41.3 mmol), bromobenzene (12.9 g, 82.5 mmol), Pd₂(dba)₃ (1.89 g, 2.06 mmol), P(t-Bu)₃ (2 mL, 8.25 mmol), and NaOtBu (7.93 g, 82.54 mmol) were added, toluene (100 mL) was added, and the resulting mixture was heated at 135° C. for 10 hours. After completing the reaction, MC and H₂O was added for extraction, and column purification was performed, thereby obtaining Intermediate 5 (10 g, 82%).

### Synthesis of Compound 2-61(F)

Intermediate 3 (10 g, 30.37 mmol), Intermediate 5 (17.87 g, 60.75 mmol), Pd(PPh₃)₄ (1.39 g, 1.52 mmol), K₂CO₃ (12.59 g, 91.13 mmol), and a 1,4-dioxane/water mixture (140 mL/35 mL) were added and heated at 120° C. for 4 hours. After completing the reaction, a solid produced after lowering the temperature to room temperature was washed with distilled water and MeOH, thereby obtaining Compound 2-61(F) (13 g, 85%).

In Preparation Example 9, Compound (F) below was synthesized in the same manner as described above, except that Compounds D and E in Table 9 below were used.

TABLE 9

| | | | | Yield |
|---|---|---|---|---|
| Compound | D | E | F | (%) |
| 2-62 | 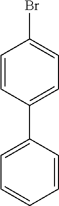 | 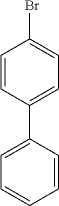 | 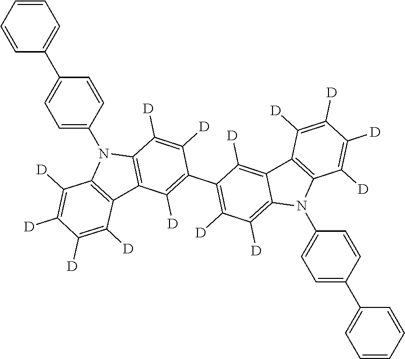 | 82 |
| 2-63 | 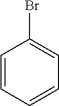 | 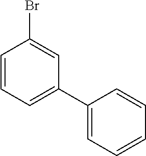 | 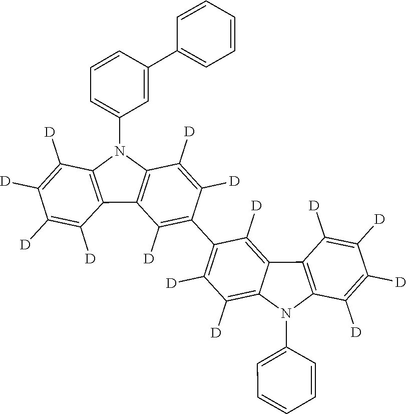 | 84 |
| 2-64 | 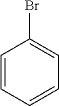 | 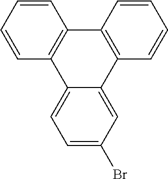 | 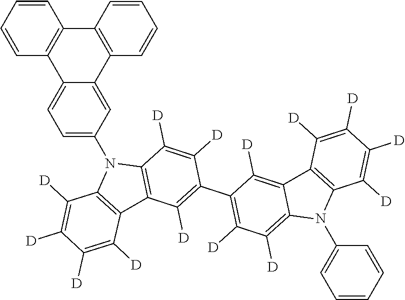 | 80 |
| 2-65 | 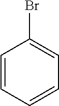 | 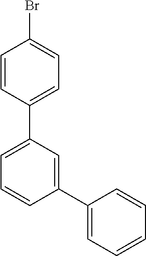 | 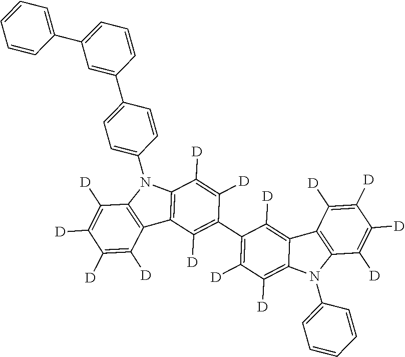 | 81 |
| 2-66 | 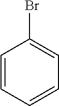 | 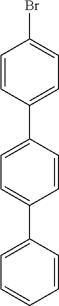 | 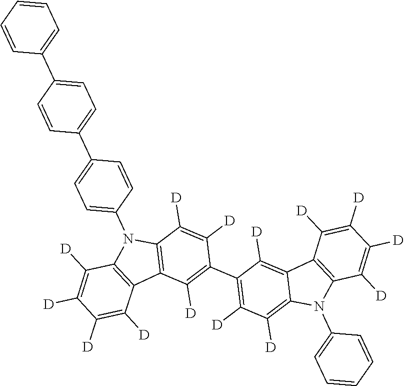 | 82 |
| 2-68 | 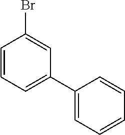 | 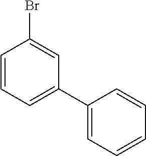 | 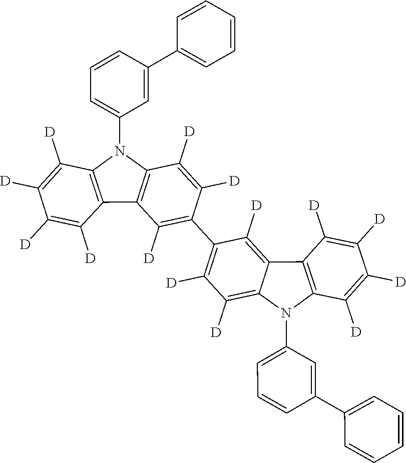 | 84 |
| 2-69 | 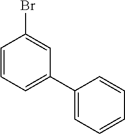 | 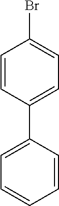 | 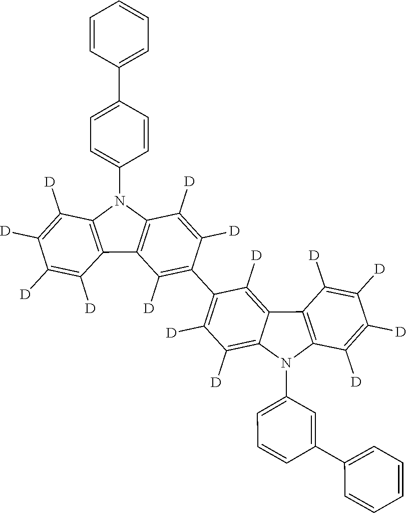 | 80 |
| 2-70 | 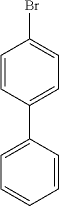 | 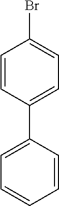 | 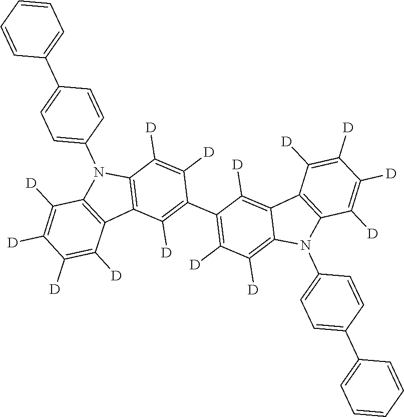 | 81 |
| 2-74 | 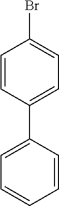 | 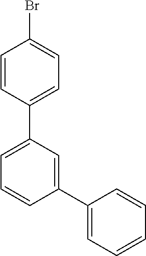 | 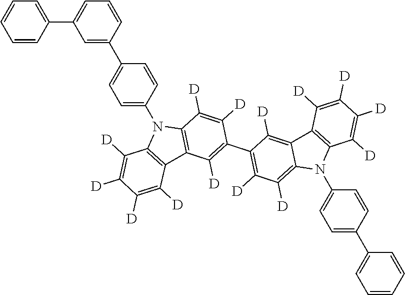 | 81 |
| 2-75 | 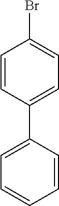 | 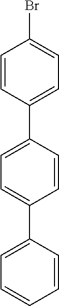 | 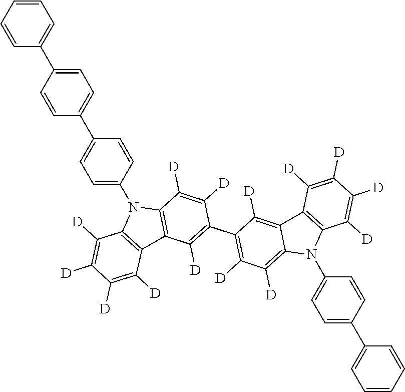 | 80 |
| 2-81 | 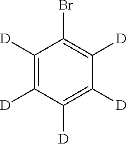 | 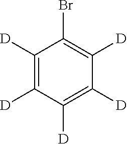 | 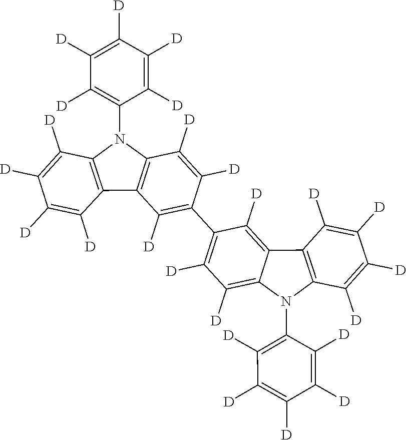 | 83 |

### [Preparation Example 10] Preparation of Compound 2-81(H)

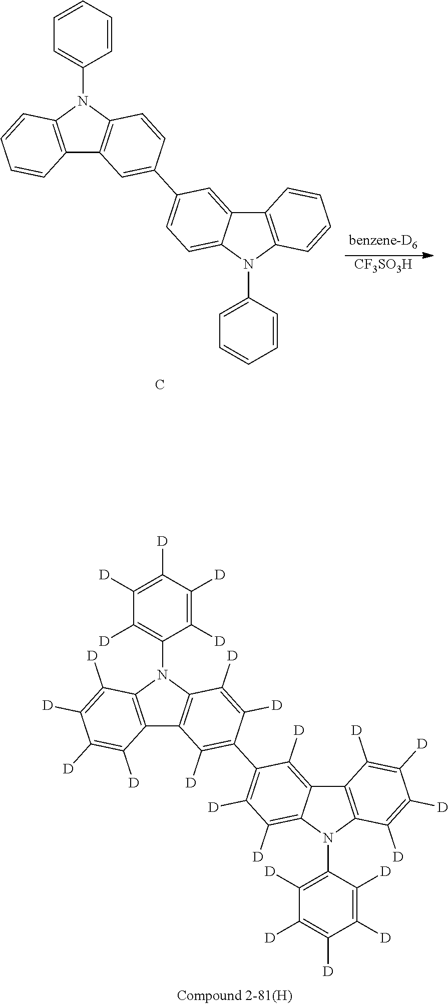

Compound G (10 g, 1 eq.), D6-benzene(500 g, 287.93 eq.), and CF₃SO₃ (245 g, 75.04 eq.) were put into a reaction flask, and reacted at 50° C. for 1 hour. After completing the reaction, H₂O was added for neutralization, MC and H₂O were added for extraction, and an organic layer was purified using a column, thereby obtaining Compound 2-81(H) (7 g, 66%).

In Preparation Example 10, Compound (H) below was synthesized in the same manner as described above, except that Compound C in Table 10 below was used.

TABLE 10

| | | | Yield |
|---|---|---|---|
| Compound | C | H | (%) |
| 2-81 | 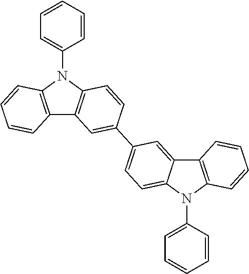 | 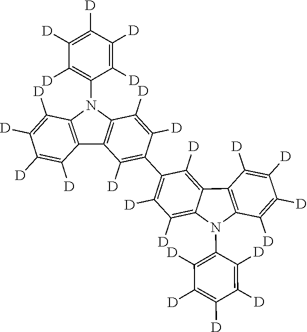 | 63 |
| 2-82 | 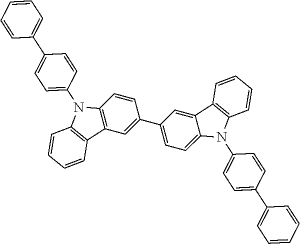 | 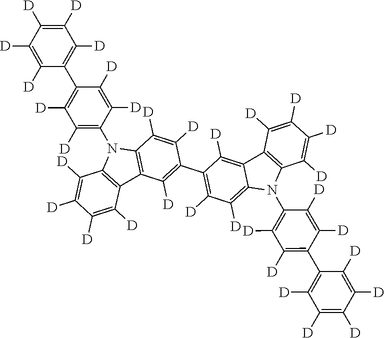 | 62 |
| 2-83 | 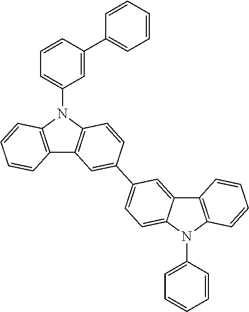 | 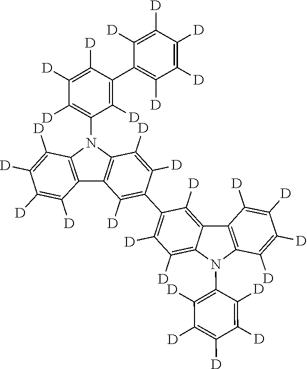 | 64 |
| 2-85 | 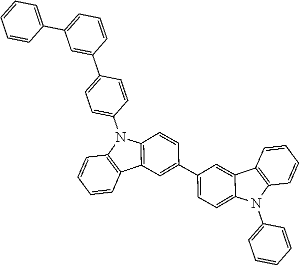 | 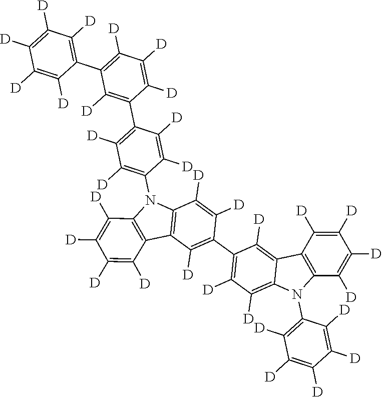 | 60 |
| 2-86 | 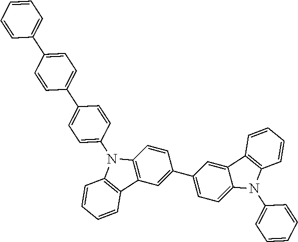 | 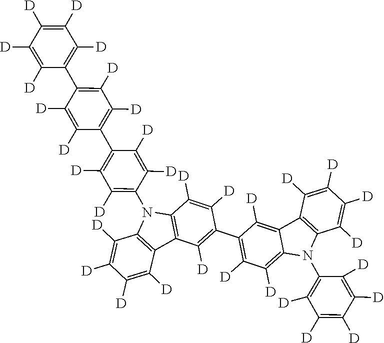 | 61 |
| 2-87 | 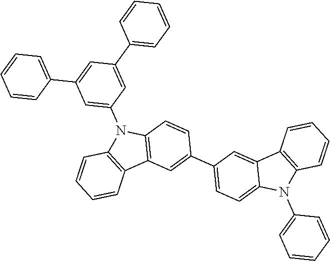 | 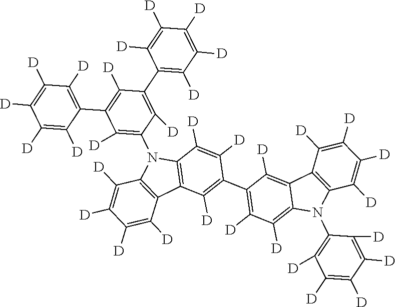 | 61 |
| 2-88 | 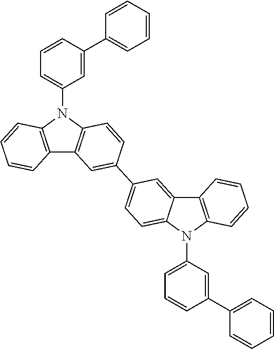 | 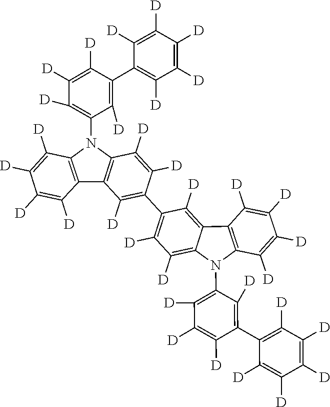 | 62 |
| 2-89 | 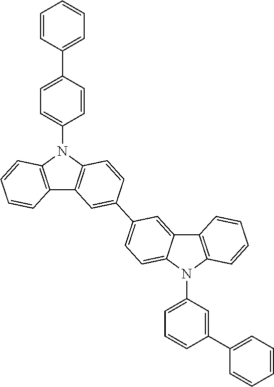 | 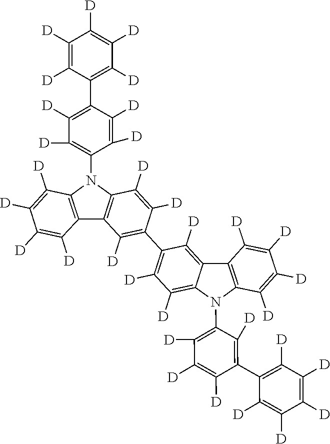 | 62 |
| 2-90 | 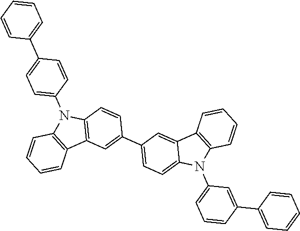 | 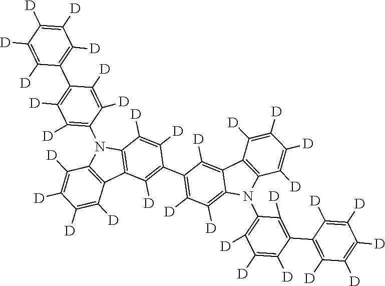 | 64 |
| 2-92 | 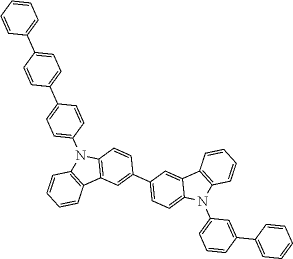 | 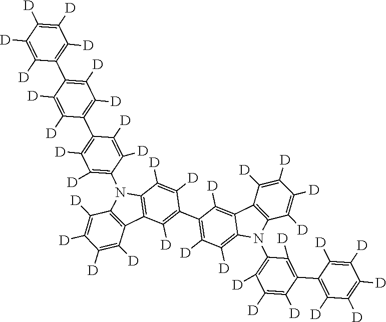 | 60 |
| 2-102 | 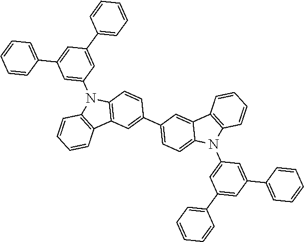 | 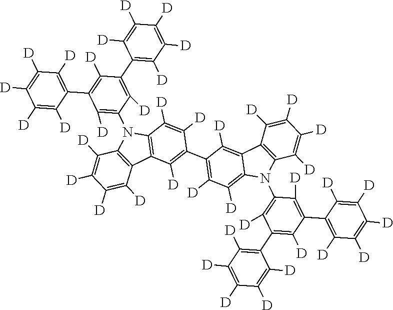 | 61 |

Compounds were prepared in the same manner as described in the preparation examples, and the synthesis confirmation results are shown in Tables 11 and 12 below. Table 11 shows the values measured by field desorption mass spectrometry (FD-MS), and Table 12 shows NMR values.

TABLE 11

| Compound | FD-Mass | Compound | FD-Mass |
|---|---|---|---|
| 1-1 | m/z = 624.73 | 1-2 | m/z = 624.73 |
| | (C43H16D9N3O2 = 624.25) | | (C43H16D9N3O2 = 624.25) |
| 1-3 | m/z = 624.73 | 1-4 | m/z = 624.73 |
| | (C43H16D9N3O2 = 624.25) | | (C43H16D9N3O2 = 624.25) |
| 1-5 | m/z = 700.83 | 1-6 | m/z = 700.83 |
| | (C49H20D9N3O2 = 700.28) | | (C49H20D9N3O2 = 700.28) |
| 1-7 | m/z = 776.92 | 1-8 | m/z = 776.92 |
| | (C55H24D9N3O2 = 776.31) | | (C55H24D9N3O2 = 776.31) |
| 1-9 | m/z = 624.73 | 1-10 | m/z = 624.73 |
| | (C43H16D9N3O2 = 624.25) | | (C43H16D9N3O2 = 624.25) |
| 1-11 | m/z = 624.73 | 1-12 | m/z = 624.73 |
| | (C43H16D9N3O2 = 624.25) | | (C43H16D9N3O2 = 624.25) |
| 1-13 | m/z = 700.83 | 1-14 | m/z = 700.83 |
| | (C49H20D9N3O2 = 700.28) | | (C49H20D9N3O2 = 700.28) |
| 1-15 | m/z = 700.83 | 1-16 | m/z = 776.92 |
| | (C49H20D9N3O2 = 700.28) | | (C55H24D9N3O2 = 776.31) |
| 1-17 | m/z = 700.83 | 1-18 | m/z = 776.92 |
| | (C49H20D9N3O2 = 700.28) | | (C55H24D9N3O2 = 776.31) |
| 1-19 | m/z = 700.83 | 1-20 | m/z = 776.92 |
| | (C49H20D9N3O2 = 700.28) | | (C55H24D9N3O2 = 776.31) |
| 1-21 | m/z = 624.73 | 1-22 | m/z = 624.73 |
| | (C43H16D9N3O2 = 624.25) | | (C43H16D9N3O2 = 624.25) |
| 1-23 | m/z = 624.73 | 1-24 | m/z = 624.73 |
| | (C43H16D9N3O2 = 624.25) | | (C43H16D9N3O2 = 624.25) |
| 1-25 | m/z = 700.83 | 1-26 | m/z = 700.83 |
| | (C49H20D9N3O2 = 700.28) | | (C49H20D9N3O2 = 700.28) |
| 1-27 | m/z = 776.92 | 1-28 | m/z = 776.92 |
| | (C55H24D9N3O2 = 776.31) | | (C55H24D9N3O2 = 776.31) |
| 1-29 | m/z = 624.73 | 1-30 | m/z = 624.73 |
| | (C43H16D9N3O2 = 624.25) | | (C43H16D9N3O2 = 624.25) |
| 1-31 | m/z = 624.73 | 1-32 | m/z = 624.73 |
| | (C43H16D9N3O2 = 624.25) | | (C43H16D9N3O2 = 624.25) |
| 1-33 | m/z = 700.83 | 1-34 | m/z = 700.83 |
| | (C49H20D9N3O2 = 700.28) | | (C49H20D9N3O2 = 700.28) |
| 1-35 | m/z = 700.83 | 1-36 | m/z = 776.92 |
| | (C49H20D9N3O2 = 700.28) | | (C55H24D9N3O2 = 776.31) |
| 1-37 | m/z = 700.83 | 1-38 | m/z = 776.92 |
| | (C49H20D9N3O2 = 700.28) | | (C55H24D9N3O2 = 776.31) |
| 1-39 | m/z = 700.83 | 1-40 | m/z = 776.92 |
| | (C49H20D9N3O2 = 700.28) | | (C55H24D9N3O2 = 776.31) |
| 1-41 | m/z = 624.73 | 1-42 | m/z = 624.73 |
| | (C43H16D9N3O2 = 624.25) | | (C43H16D9N3O2 = 624.25) |
| 1-43 | m/z = 624.73 | 1-44 | m/z = 624.73 |
| | (C43H16D9N3O2 = 624.25) | | (C43H16D9N3O2 = 624.25) |
| 1-45 | m/z = 700.83 | 1-46 | m/z = 700.83 |
| | (C49H20D9N3O2 = 700.28) | | (C49H20D9N3O2 = 700.28) |
| 1-47 | m/z = 776.92 | 1-48 | m/z = 776.92 |
| | (C55H24D9N3O2 = 776.31) | | (C55H24D9N3O2 = 776.31) |
| 1-49 | m/z = 624.73 | 1-50 | m/z = 624.73 |
| | (C43H16D9N3O2 = 624.25) | | (C43H16D9N3O2 = 624.25) |
| 1-51 | m/z = 624.73 | 1-52 | m/z = 624.73 |
| | (C43H16D9N3O2 = 624.25) | | (C43H16D9N3O2 = 624.25) |
| 1-53 | m/z = 700.83 | 1-54 | m/z = 700.83 |
| | (C49H20D9N3O2 = 700.28) | | (C49H20D9N3O2 = 700.28) |
| 1-55 | m/z = 700.83 | 1-56 | m/z = 776.92 |
| | (C49H20D9N3O2 = 700.28) | | (C55H24D9N3O2 = 776.31) |
| 1-57 | m/z = 700.83 | 1-58 | m/z = 776.92 |
| | (C49H20D9N3O2 = 700.28) | | (C55H24D9N3O2 = 776.31) |
| 1-59 | m/z = 700.83 | 1-60 | m/z = 776.92 |
| | (C49H20D9N3O2 = 700.28) | | (C55H24D9N3O2 = 776.31) |
| 1-61 | m/z = 624.73 | 1-62 | m/z = 624.73 |
| | (C43H16D9N3O2 = 624.25) | | (C43H16D9N3O2 = 624.25) |
| 1-63 | m/z = 624.73 | 1-64 | m/z = 624.73 |
| | (C43H16D9N3O2 = 624.25) | | (C43H16D9N3O2 = 624.25) |
| 1-65 | m/z = 700.83 | 1-66 | m/z = 700.83 |
| | (C49H20D9N3O2 = 700.28) | | (C49H20D9N3O2 = 700.28) |
| 1-67 | m/z = 776.92 | 1-68 | m/z = 776.92 |
| | (C55H24D9N3O2 = 776.31) | | (C55H24D9N3O2 = 776.31) |
| 1-69 | m/z = 624.73 | 1-70 | m/z = 624.73 |
| | (C43H16D9N3O2 = 624.25) | | (C43H16D9N3O2 = 624.25) |
| 1-71 | m/z = 624.73 | 1-72 | m/z = 624.73 |
| | (C43H16D9N3O2 = 624.25) | | (C43H16D9N3O2 = 624.25) |
| 1-73 | m/z = 700.83 | 1-74 | m/z = 700.83 |
| | (C49H20D9N3O2 = 700.28) | | (C49H20D9N3O2 = 700.28) |
| 1-75 | m/z = 700.83 | 1-76 | m/z = 776.92 |
| | (C49H20D9N3O2 = 700.28) | | (C55H24D9N3O2 = 776.31) |
| 1-77 | m/z = 700.83 | 1-78 | m/z = 776.92 |
| | (C49H20D9N3O2 = 700.28) | | (C55H24D9N3O2 = 776.31) |
| 1-79 | m/z = 700.83 | 1-80 | m/z = 776.92 |
| | (C49H20D9N3O2 = 700.28) | | (C55H24D9N3O2 = 776.31) |
| 1-81 | m/z = 624.73 | 1-82 | m/z = 624.73 |
| | (C43H16D9N3O2 = 624.25) | | (C43H16D9N3O2 = 624.25) |
| 1-83 | m/z = 624.73 | 1-84 | m/z = 624.73 |
| | (C43H16D9N3O2 = 624.25) | | (C43H16D9N3O2 = 624.25) |
| 1-85 | m/z = 700.83 | 1-86 | m/z = 700.83 |
| | (C49H20D9N3O2 = 700.28) | | (C49H20D9N3O2 = 700.28) |
| 1-87 | m/z = 776.92 | 1-88 | m/z = 776.92 |
| | (C55H24D9N3O2 = 776.31) | | (C55H24D9N3O2 = 776.31) |
| 1-89 | m/z = 624.73 | 1-90 | m/z = 624.73 |
| | (C43H16D9N3O2 = 624.25) | | (C43H16D9N3O2 = 624.25) |
| 1-91 | m/z = 624.73 | 1-92 | m/z = 624.73 |
| | (C43H16D9N3O2 = 624.25) | | (C43H16D9N3O2 = 624.25) |
| 1-93 | m/z = 700.83 | 1-94 | m/z = 700.83 |
| | (C49H20D9N3O2 = 700.28) | | (C49H20D9N3O2 = 700.28) |
| 1-95 | m/z = 700.83 | 1-96 | m/z = 776.92 |
| | (C49H20D9N3O2 = 700.28) | | (C55H24D9N3O2 = 776.31) |
| 1-97 | m/z = 700.83 | 1-98 | m/z = 776.92 |
| | (C49H20D9N3O2 = 700.28) | | (C55H24D9N3O2 = 776.31) |
| 1-99 | m/z = 700.83 | 1-100 | m/z = 776.92 |
| | (C49H20D9N3O2 = 700.28) | | (C55H24D9N3O2 = 776.31) |
| 1-101 | m/z = 640.80 | 1-102 | m/z = 640.80 |
| | (C49H16D9N3OS = 640.23) | | (C49H16D9N3OS = 640.23) |
| 1-103 | m/z = 640.80 | 1-104 | m/z = 640.80 |
| | (C49H16D9N3OS = 640.23) | | (C49H16D9N3OS = 640.23) |
| 1-105 | m/z = 716.89 | 1-106 | m/z = 716.89 |
| | (C49H20D9N3OS = 716.26) | | (C49H20D9N3OS = 716.26) |
| 1-107 | m/z = 792.99 | 1-108 | m/z = 792.99 |
| | (C55H24D9N3OS = 792.29) | | (C55H24D9N3OS = 792.29) |
| 1-109 | m/z = 640.80 | 1-110 | m/z = 640.80 |
| | (C49H16D9N3OS = 640.23) | | (C49H16D9N3OS = 640.23) |
| 1-111 | m/z = 640.80 | 1-112 | m/z = 640.80 |
| | (C49H16D9N3OS = 640.23) | | (C49H16D9N3OS = 640.23) |
| 1-113 | m/z = 716.89 | 1-114 | m/z = 792.99 |
| | (C49H20D9N3OS = 716.26) | | (C55H24D9N3OS = 792.29) |
| 1-115 | m/z = 716.89 | 1-116 | m/z = 792.99 |
| | (C49H20D9N3OS = 716.26) | | (C55H24D9N3OS = 792.29) |
| 1-117 | m/z = 716.89 | 1-118 | m/z = 792.99 |
| | (C49H20D9N3OS = 716.26) | | (C55H24D9N3OS = 792.29) |
| 1-119 | m/z = 716.89 | 1-120 | m/z = 792.99 |
| | (C49H20D9N3OS = 716.26) | | (C55H24D9N3OS = 792.29) |
| 1-121 | m/z = 640.80 | 1-122 | m/z = 640.80 |
| | (C49H16D9N3OS = 640.23) | | (C49H16D9N3OS = 640.23) |
| 1-123 | m/z = 640.80 | 1-124 | m/z = 640.80 |
| | (C49H16D9N3OS = 640.23) | | (C49H16D9N3OS = 640.23) |
| 1-125 | m/z = 716.89 | 1-126 | m/z = 716.89 |
| | (C49H20D9N3OS = 716.26) | | (C49H20D9N3OS = 716.26) |
| 1-127 | m/z = 792.99 | 1-128 | m/z = 792.99 |
| | (C55H24D9N3OS = 792.29) | | (C55H24D9N3OS = 792.29) |
| 1-129 | m/z = 640.80 | 1-130 | m/z = 640.80 |
| | (C49H16D9N3OS = 640.23) | | (C49H16D9N3OS = 640.23) |
| 1-131 | m/z = 640.80 | 1-132 | m/z = 640.80 |
| | (C49H16D9N3OS = 640.23) | | (C49H16D9N3OS = 640.23) |
| 1-133 | m/z = 716.89 | 1-134 | m/z = 792.99 |
| | (C49H20D9N3OS = 716.26) | | (C55H24D9N3OS = 792.29) |
| 1-135 | m/z = 716.89 | 1-136 | m/z = 792.99 |
| | (C49H20D9N3OS = 716.26) | | (C55H24D9N3OS = 792.29) |
| 1-137 | m/z = 716.89 | 1-138 | m/z = 792.99 |
| | (C49H20D9N3OS = 716.26) | | (C55H24D9N3OS = 792.29) |
| 1-139 | m/z = 716.89 | 1-140 | m/z = 792.99 |
| | (C49H20D9N3OS = 716.26) | | (C55H24D9N3OS = 792.29) |
| 1-141 | m/z = 640.80 | 1-142 | m/z = 640.80 |
| | (C49H16D9N3OS = 640.23) | | (C49H16D9N3OS = 640.23) |
| 1-143 | m/z = 640.80 | 1-144 | m/z = 640.80 |
| | (C49H16D9N3OS = 640.23) | | (C49H16D9N3OS = 640.23) |
| 1-145 | m/z = 716.89 | 1-146 | m/z = 716.89 |
| | (C49H20D9N3OS = 716.26) | | (C49H20D9N3OS = 716.26) |
| 1-147 | m/z = 792.99 | 1-148 | m/z = 792.99 |
| | (C55H24D9N3OS = 792.29) | | (C55H24D9N3OS = 792.29) |
| 1-149 | m/z = 640.80 | 1-150 | m/z = 640.80 |
| | (C49H16D9N3OS = 640.23) | | (C49H16D9N3OS = 640.23) |
| 1-151 | m/z = 640.80 | 1-152 | m/z = 640.80 |
| | (C49H16D9N3OS = 640.23) | | (C49H16D9N3OS = 640.23) |
| 1-153 | m/z = 716.89 | 1-154 | m/z = 792.99 |
| | (C49H20D9N3OS = 716.26) | | (C55H24D9N3OS = 792.29) |
| 1-155 | m/z = 716.89 | 1-156 | m/z = 792.99 |
| | (C49H20D9N3OS = 716.26) | | (C55H24D9N3OS = 792.29) |
| 1-157 | m/z = 716.89 | 1-158 | m/z = 792.99 |
| | (C49H20D9N3OS = 716.26) | | (C55H24D9N3OS = 792.29) |
| 1-159 | m/z = 716.89 | 1-160 | m/z = 792.99 |
| | (C49H20D9N3OS = 716.26) | | (C55H24D9N3OS = 792.29) |
| 1-161 | m/z = 640.80 | 1-162 | m/z = 640.80 |
| | (C49H16D9N3OS = 640.23) | | (C49H16D9N3OS = 640.23) |
| 1-163 | m/z = 640.80 | 1-164 | m/z = 640.80 |
| | (C49H16D9N3OS = 640.23) | | (C49H16D9N3OS = 640.23) |
| 1-165 | m/z = 716.89 | 1-166 | m/z = 716.89 |
| | (C49H20D9N3OS = 716.26) | | (C49H20D9N3OS = 716.26) |
| 1-167 | m/z = 792.99 | 1-168 | m/z = 792.99 |
| | (C55H24D9N3OS = 792.29) | | (C55H24D9N3OS = 792.29) |
| 1-169 | m/z = 640.80 | 1-170 | m/z = 640.80 |
| | (C49H16D9N3OS = 640.23) | | (C49H16D9N3OS = 640.23) |
| 1-171 | m/z = 640.80 | 1-172 | m/z = 640.80 |
| | (C49H16D9N3OS = 640.23) | | (C49H16D9N3OS = 640.23) |
| 1-173 | m/z = 716.89 | 1-174 | m/z = 792.99 |
| | (C49H20D9N3OS = 716.26) | | (C55H24D9N3OS = 792.29) |
| 1-175 | m/z = 716.89 | 1-176 | m/z = 792.99 |
| | (C49H20D9N3OS = 716.26) | | (C55H24D9N3OS = 792.29) |
| 1-177 | m/z = 716.89 | 1-178 | m/z = 792.99 |
| | (C49H20D9N3OS = 716.26) | | (C55H24D9N3OS = 792.29) |
| 1-179 | m/z = 716.89 | 1-180 | m/z = 792.99 |
| | (C49H20D9N3OS = 716.26) | | (C55H24D9N3OS = 792.29) |
| 1-181 | m/z = 640.80 | 1-182 | m/z = 640.80 |
| | (C49H16D9N3OS = 640.23) | | (C49H16D9N3OS = 640.23) |
| 1-183 | m/z = 640.80 | 1-184 | m/z = 640.80 |
| | (C49H16D9N3OS = 640.23) | | (C49H16D9N3OS = 640.23) |
| 1-185 | m/z = 716.89 | 1-186 | m/z = 716.89 |
| | (C49H20D9N3OS = 716.26) | | (C49H20D9N3OS = 716.26) |
| 1-187 | m/z = 792.99 | 1-188 | m/z = 792.99 |
| | (C55H24D9N3OS = 792.29) | | (C55H24D9N3OS = 792.29) |
| 1-189 | m/z = 640.80 | 1-190 | m/z = 640.80 |
| | (C49H16D9N3OS = 640.23) | | (C49H16D9N3OS = 640.23) |
| 1-191 | m/z = 640.80 | 1-192 | m/z = 640.80 |
| | (C49H16D9N3OS = 640.23) | | (C49H16D9N3OS = 640.23) |
| 1-193 | m/z = 716.89 | 1-194 | m/z = 792.99 |
| | (C49H20D9N3OS = 716.26) | | (C55H24D9N3OS = 792.29) |
| 1-195 | m/z = 716.89 | 1-196 | m/z = 792.99 |
| | (C49H20D9N3OS = 716.26) | | (C55H24D9N3OS = 792.29) |
| 1-197 | m/z = 716.89 | 1-198 | m/z = 792.99 |
| | (C49H20D9N3OS = 716.26) | | (C55H24D9N3OS = 792.29) |
| 1-199 | m/z = 716.89 | 1-200 | m/z = 792.99 |
| | (C49H20D9N3OS = 716.26) | | (C55H24D9N3OS = 792.29) |
| 1-201 | m/z = 640.80 | 1-202 | m/z = 640.80 |
| | (C49H16D9N3OS = 640.23) | | (C49H16D9N3OS = 640.23) |
| 1-203 | m/z = 640.80 | 1-204 | m/z = 640.80 |
| | (C49H16D9N3OS = 640.23) | | (C49H16D9N3OS = 640.23) |
| 1-205 | m/z = 716.89 | 1-206 | m/z = 716.89 |
| | (C49H20D9N3OS = 716.26) | | (C49H20D9N3OS = 716.26) |
| 1-207 | m/z = 792.99 | 1-208 | m/z = 792.99 |
| | (C55H24D9N3OS = 792.29) | | (C55H24D9N3OS = 792.29) |
| 1-209 | m/z = 716.89 | 1-210 | m/z = 792.99 |
| | (C49H20D9N3OS = 716.26) | | (C55H24D9N3OS = 792.29) |
| 1-211 | m/z = 716.89 | 1-212 | m/z = 792.99 |
| | (C49H20D9N3OS = 716.26) | | (C55H24D9N3OS = 792.29) |
| 1-213 | m/z = 716.89 | 1-214 | m/z = 716.89 |
| | (C49H20D9N3OS = 716.26) | | (C49H20D9N3OS = 716.26) |
| 1-215 | m/z = 792.99 | 1-216 | m/z = 792.99 |
| | (C55H24D9N3OS = 792.29) | | (C55H24D9N3OS = 792.29) |
| 1-217 | m/z = 640.80 | 1-218 | m/z = 640.80 |
| | (C49H16D9N3OS = 640.23) | | (C49H16D9N3OS = 640.23) |
| 1-219 | m/z = 716.89 | 1-220 | m/z = 792.99 |
| | (C49H20D9N3OS = 716.26) | | (C55H24D9N3OS = 792.29) |
| 1-221 | m/z = 640.80 | 1-222 | m/z = 716.89 |
| | (C49H16D9N3OS = 640.23) | | (C49H20D9N3OS = 716.26) |

TABLE 12

| Compound | ¹H NMR(CDCl3, 300 Mz) |
|---|---|
| 1-1 | δ = 8.28(m, 2H), 7.95(d, 1H), 7.79-7.75(m, 4H), 7.64-7.62(m, 2H), 7.51-7.41(m, 7H) |
| 1-8 | δ = 8.24(d, 1H), 7.95(m, 1H), 7.79-7.70(m, 5H), 7.64-7.41(m, 13H), 7.25(s, 4H) |
| 1-10 | δ = 8.28(m, 2H), 7.89-7.79(m, 5H), 7.66(d, 1H), 7.51-7.32(m, 8H) |
| 1-16 | δ = 8.24(m, 2H), 7.95(d, 1H), 7.79-7.41(m, 21H) |
| 1-19 | δ = 8.28(m, 2H), 7.95(d, 1H), 7.85-7.75(m, 5H), 7.64(s, 1H), 7.52-7.41(m, 9H), 7.25(d, 2H) |
| 1-22 | δ = 8.28(m, 2H), 7.95(d, 1H), 7.81-7.71(m, 4H), 7.64(s, 1H), 7.52-7.41(m, 8H) |
| 1-26 | δ = 7.95(d, 1H), 7.85-7.75(m, 6H), 7.64-7.62(m, 2H), 7.52-7.41(m, 9H), 7.25(m, 2H) |
| 1-34 | δ = 8.24(m, 1H), 7.95(m, 1H), 7.85-7.70(m, 7H), 7.64-7.61(m, 13H), 7.25(m, 2H) |
| 1-37 | δ = 8.28(m, 2H), 7.85-7.79(m, 4H), 7.66-7.25(m, 13H) |
| 1-46 | δ = 7.95(m, 1H), 7.85-7.75(m, 6H), 7.64-7.62(m, 2H), 7.52-7.41(m, 9H), 7.25(m, 2H) |
| 1-55 | δ = 8.28-8.24(m, 3H), 7.95(d, 1H), 7.79-7.41(m, 16H) |
| 1-63 | δ = 8.28(d, 2H), 7.95(m, 2H), 7.75(m, 2H), 7.64(s, 2H), 7.52-7.41(m, 8H) |
| 1-73 | δ = 8.28(m, 2H), 7.95(m, 1H), 7.85-7.75(m, 6H), 7.64-7.62(m, 2H), 7.51-7.41(m, 7H), 7.25(m, 2H) |
| 1-84 | δ = 8.28(m, 2H), 7.95(m, 1H), 7.85-7.75(m, 3H), 7.64(s, 1H), 7.52-7.38(m, 9H) |
| 1-94 | δ = 8.24(m, 4H), 7.95(m, 1H), 7.85-7.70(m, 7H), 7.64-7.41(m, 13H), 7.25(m, 2H) |
| 1-101 | δ = 8.28(m, 2H), 8.11-8.08(m, 2H), 7.94(d, 1H), 7.82-7.79(m, 4H), 7.56-7.41(m, 7H) |
| 1-113 | δ = 8.28(m, 2H), 8.11-8.08(m, 2H), 7.94(d, 1H), 7.85-7.79(m, 6H), 7.56-7.41(m, 7H), 7.25(m, 2H) |
| 1-122 | δ = 8.28(m, 2H), 8.11-8.08(m, 2H), 8.00(m, 2H), 7.86-7.82(m, 2H), 7.52-7.41(m, 8H) |
| 1-135 | δ = 8.28(m, 2H), 8.11-8.08(m, 2H), 7.94(d, 1H), 7.82-7.79(m, 4H), 7.70(s, 1H), 7.57-7.41(m, 9H) |
| 1-149 | δ = 8.45(d, 1H), 8.28(m, 2H), 8.04-7.98(m, 2H), 7.79-7.77(m, 3H), 7.51-7.41(m, 8H) |
| 1-165 | δ = 8.24(m, 2H), 8.11-8.08(m, 2H), 7.94(d, 1H), 7.82-7.79(m, 4H), 7.70(s, 1H), 7.57-7.41(m, 11H) |
| 2-3 | δ = 8.55(d, 1H), 8.18-8.09(m, 3H), 8.00-7.87(m, 3H), 7.77(s, 2H), 7.58-7.25(m, 18H) |
| 2-4 | δ = 8.55(d, 1H), 8.18-8.12(m, 2H), 8.00-7.84(m, 3H), 7.79-7.77(m, 4H), 7.68-7.25(m, 22H) |
| 2-7 | δ = 8.55(d, 1H), 8.18-8.09(m, 4H), 8.00-7.94(m, 2H), 7.87(m, 1H), 7.77(m, 2H), 7.69- |
| | 7.63(m, 2H), 7.52-7.25(m, 20H) |
| 2-27 | δ = 8.55(m, 1H), 8.18-8.09(m, 4h), 8.00-7.94(m, 2H), 7.77(m, 2H) |
| 2-28 | δ = 8.55(m, 1H), 8.18-8.09(m, 3H), 8.00-8.79(m, 2H), 7.87(m, 1H), 7.79-7.77(m, 4H), |
| | 7.69-7.63(m, 4H), 7.52-7.25(m, 12H) |
| 2-29 | δ = 8.55(m, 1H), 8.18-8.09(m, 3H), 8.00-7.94(m, 2H0, 7.87(m, 1H), 7.87(m, 1H), 7.79- |
| | 7.77(m, 4H), 7.69-7.63(m, 4H), 7.52-7.25(m, 21H) |
| 2-32 | δ = 8.55(m, 1H), 8.18-8.12(m, 2H), 8.00-7.87(m, 3H), 7.79-7.77(m, 6H), 7.69-7.63(m, |
| | 6H), 7.52-7.25(m, 14H) |
| 2-34 | δ = 8.55(m, 1H), 8.18-8.12(m, 2H), 8.00-7.87(m, 3H), 7.79-7.77(m, 6H), 7.67-7.63(m, |
| | 6H), 7.52-7.25(m, 18H) |
| 2-38 | δ = 8.55(m, 1H), 8.18-8.12(m, 2H), 8.05-7.87(m, 6H), 7.79-7.77(m, 4H), 7.69-7.63(m, |
| | 4H), 7.52-7.25(m, 23H) |
| 2-46 | δ = 8.55(d, 1H), 8.18-8.12(m, 2H), 8.00-7.87(m, 3H), 7.77-7.63(m, 4H), 7.50(m, 1H), |
| | 7.33-7.25(m, 3H) |
| 2-49 | δ = 8.55(d, 1H), 8.18-8.12(m, 2H), 8.00-7.87(m, 3H), 7.77-7.63(m, 4H), 7.50(m, 1H), |
| | 7.33-7.25(m, 3H) |
| 2-62 | δ = 7.79(m, 4H), 7.68(m. 4H), 7.52-7.41(m, 10H) |
| 2-65 | δ = 7.79(m, 2H), 7.70-7.68(m, 3H), 7.58-7.41(m, 13H) |
| 2-70 | δ = 7.79(m, 4H), 7.68(m, 4H), 7.52-7.41(m, 10H) |

### Experimental Example 1

1) Manufacture of Organic Light Emitting Device

A glass substrate on which an ITO thin film was coated to a thickness of 1,500 Å was cleaned with ultrasonic waves in distilled water. After distilled water cleaning, ultrasonic cleaning was performed with a solvent such as acetone, methanol, or isopropyl alcohol followed by drying, and then UVO treatment was performed in an UV cleaner for 5 minutes using UV. Afterward, the substrate was transferred to a plasma cleaner (PT), subjected to plasma treatment to remove the ITP work function and a residual film and then transferred to thermal deposition equipment for organic deposition.

On the ITO transparent electrode (positive electrode), common layers, such as a hole injection layer (4,4′,4″-Tris[2-naphthyl(phenyl)amino]triphenylamine (2-TNATA)) and a hole transport layer (N,N′-Di(1-naphthyl)-N,N′-diphenyl-(1,1′-biphenyl)-4,4′-diamine (NPB), were formed.

An emissive layer was formed thereon through thermal vacuum deposition as follows. The emissive layer used a compound shown in Table 13 below as a host and tris(2-phenylpyridine)iridium (Ir(ppy)₃) as a green phosphorescent dopant, and the host was doped with 7% Ir(ppy)₃ and deposited to a thickness of 400 Å. Afterward, bathocuproine (BCP) was deposited to a thickness of 60 Å as a hole blocking layer, and Alq₃ was deposited thereon to a thickness of 200 Å as an electron transport layer. Finally, after an electron injection layer was formed on the electron transport layer by depositing lithium fluoride (LiF) to a thickness of 10 Å, an aluminum (Al) negative electrode was formed on the electron injection layer by depositing aluminum (Al) to a thickness of 1,200 Å, thereby manufacturing an organic electroluminescent device.

In Table 13 below, Examples and Comparative Examples other than those separately denoted as red hosts were used as green hosts

In the case of Examples used as red hosts, during the formation of an emissive layer, a red phosphorescent dopant Ir(piq)₂(acac) was used instead of a green phosphorescent dopant, and the organic electroluminescent device was manufactured in the same manner as described above, except that, instead of a green phosphorescent dopant, a red phosphorescent dopant Ir(piq)₂(acac) was used to form an emissive layer and bathophenanthroline (BPhen), instead of BCP, was deposited to 30 Å.

Meanwhile, all organic compounds required for manufacturing an OLED were used in the manufacture of an OLED after purification of each material by vacuum sublimination under 10⁻⁸ to 10⁻⁶ torr.

### COMPARATIVE EXAMPLE

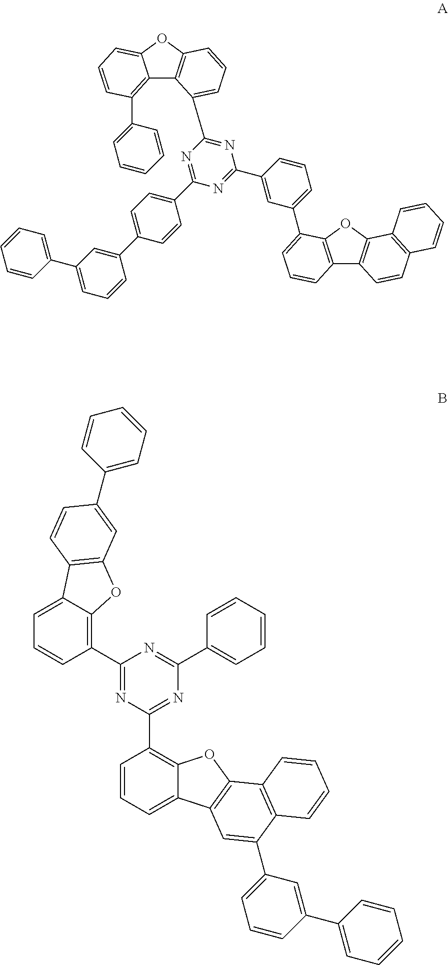 [SMILES: c1ccc(-c2cccc(-c3ccc(-c4nc(-c5cccc(-c6cccc7c6oc6c8ccccc8ccc76)c5)nc(-c5cccc6oc7cccc(-c8ccccc8)c7c56)n4)cc3)c2)cc1 ; c1ccc(-c2cccc(-c3cc4c5cccc(-c6nc(-c7ccccc7)nc(-c7cccc8c7oc7cc(-c9ccccc9)ccc78)n6)c5oc4c4ccccc34)c2)cc1] 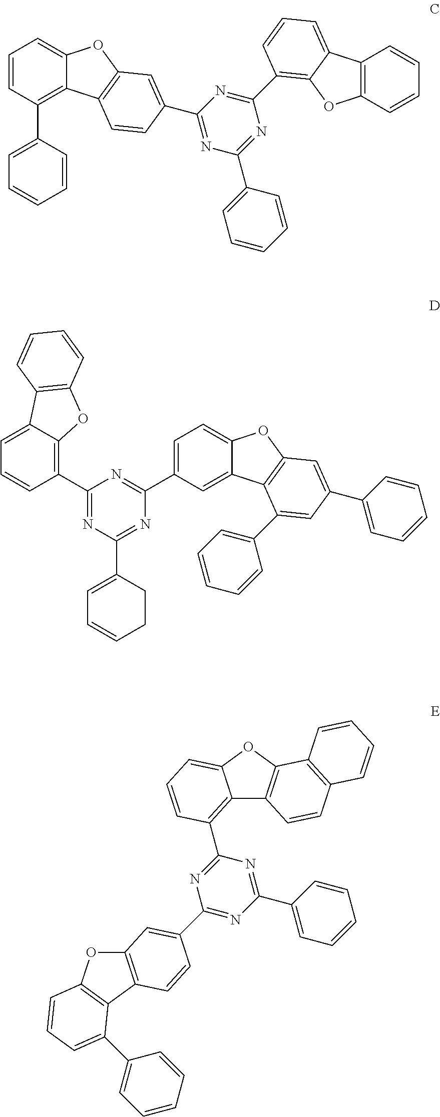 [SMILES: c1ccc(-c2nc(-c3ccc4c(c3)oc3cccc(-c5ccccc5)c34)nc(-c3cccc4c3oc3ccccc34)n2)cc1 ; C1=CCCC(c2nc(-c3ccc4oc5cc(-c6ccccc6)cc(-c6ccccc6)c5c4c3)nc(-c3cccc4c3oc3ccccc34)n2)=C1 ; c1ccc(-c2nc(-c3ccc4c(c3)oc3cccc(-c5ccccc5)c34)nc(-c3cccc4oc5c6ccccc6ccc5c34)n2)cc1] 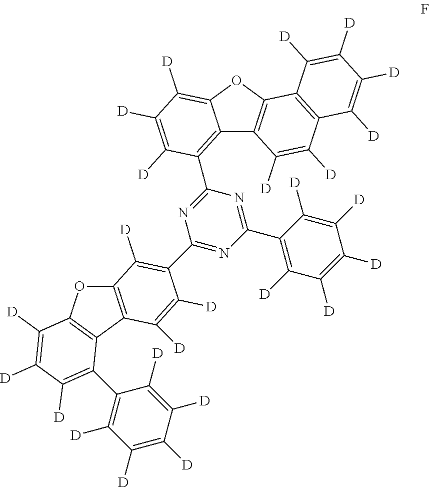 [SMILES: [2H]c1c([2H])c([2H])c(-c2nc(-c3c([2H])c([2H])c4c(oc5c([2H])c([2H])c([2H])c(-c6c([2H])c([2H])c([2H])c([2H])c6[2H])c54)c3[2H])nc(-c3c([2H])c([2H])c([2H])c4oc5c6c([2H])c([2H])c([2H])c([2H])c6c([2H])c([2H])c5c34)n2)c([2H])c1[2H]] [0283] 2) Driving Voltage and Luminous Efficiency of Electroluminescent Device

Electrolumines cent (EL) characteristics of the organic electroluminescent device manufactured as described above were measured using M7000 (McScience), and with the measurement result, T₉₀ was measured when the standard luminance was 6,000 cd/m² using lifetime measurement equipment (M6000, McScience).

The results of measuring the driving voltage, luminous efficiency, color coordinates (CIE), and lifetime of the organic light emitting device manufactured according to the present application are shown in Table 13 below.

TABLE 13

| | | Driving | Efficiency | CIE | Lifetime |
|---|---|---|---|---|---|
| | Compound | voltage (V) | (cd/A) | (x, y) | (T₉₀) |
| Comparative | A | 6.01 | 60.3 | (0.286, 0.686) | 89 |
| Example 1 | | | | | |
| Comparative | B | 6.62 | 65.4 | (0.274, 0.678) | 90 |
| Example 2 | | | | | |
| Comparative | C | 6.32 | 66.2 | (0.279, 0.677) | 91 |
| Example 3 | | | | | |
| Comparative | D | 6.88 | 61.7 | (0.278, 0.677) | 90 |
| Example 4 | | | | | |
| Comparative | E | 5.54 | 78.9 | (0.271, 0.671) | 89 |
| Example 5 | | | | | |
| Comparative | F | 5.32 | 81.2 | (0.275, 0.672) | 92 |
| Example 6 | | | | | |
| Example 1 | 1-1 | 4.58 | 96.7 | (0.277, 0.669) | 116 |
| Example 2 | 1-2 | 4.62 | 90.3 | (0.281, 0.679) | 108 |
| Example 3 | 1-3 | 4.63 | 92.7 | (0.280, 0.677) | 105 |
| Example 4 | 1-4 | 4.64 | 91.4 | (0.279, 0.676) | 107 |
| Example 5 | 1-5 | 4.60 | 96.2 | (0.272, 0.669) | 113 |
| Example 6 | 1-6 | 4.59 | 97.3 | (0.271, 0.671) | 116 |
| Example 7 | 1-7 | 4.58 | 94.2 | (0.275, 0.672) | 114 |
| Example 8 | 1-8 | 4.59 | 95.8 | (0.279, 0.675) | 118 |
| Example 9 | 1-9 | 4.71 | 88.9 | (0.280, 0.676) | 197 |
| Example 10 | 1-10 | 4.72 | 89.2 | (0.278, 0.672) | 98 |
| Example 11 | 1-11 | 4.75 | 90.3 | (0.283, 0.687) | 99 |
| Example 12 | 1-12 | 4.80 | 88.7 | (0.286, 0.686) | 98 |
| Example 13 | 1-13 | 4.57 | 95.9 | (0.274, 0.678) | 114 |
| Example 14 | 1-14 | 4.58 | 97.7 | (0.279, 0.677) | 113 |
| Example 15 | 1-15 | 4.59 | 97.8 | (0.278, 0.677) | 119 |
| Example 16 | 1-16 | 4.57 | 96.3 | (0.276, 0.671) | 118 |
| Example 17 | 1-17 | 4.77 | 88.3 | (0.284, 0.687) | 98 |
| Example 18 | 1-18 | 4.72 | 87.2 | (0.281, 0.684) | 99 |
| Example 19 | 1-19 | 4.70 | 86.3 | (0.283, 0.681) | 97 |
| Example 20 | 1-20 | 4.71 | 89.3 | (0.279, 0.681) | 99 |
| Example 21 | 1-21 | 4.59 | 96.8 | (0.280, 0.679) | 115 |
| Example 22 | 1-22 | 4.63 | 90.7 | (0.281, 0.678) | 109 |
| Example 23 | 1-23 | 4.62 | 93.7 | (0.278, 0.682) | 104 |
| Example 24 | 1-24 | 4.63 | 92.4 | (0.286, 0.692) | 106 |
| Example 25 | 1-25 | 4.61 | 96.2 | (0.274, 0.672) | 116 |
| Example 26 | 1-26 | 4.59 | 97.3 | (0.278, 0.678) | 113 |
| Example 27 | 1-27 | 4.59 | 94.7 | (0.277, 0.674) | 112 |
| Example 28 | 1-28 | 4.60 | 96.8 | (0.279, 0.674) | 119 |
| Example 29 | 1-29 | 4.72 | 87.9 | (0.278, 0.677) | 96 |
| Example 30 | 1-30 | 4.71 | 88.2 | (0.279, 0.681) | 99 |
| Example 31 | 1-31 | 4.76 | 90.2 | (0.280, 0.679) | 98 |
| Example 32 | 1-32 | 4.79 | 89.7 | (0.281, 0.678) | 98 |
| Example 33 | 1-33 | 4.58 | 96.9 | (0.278, 0.682) | 112 |
| Example 34 | 1-34 | 4.57 | 98.7 | (0.286, 0.692) | 114 |
| Example 35 | 1-35 | 4.59 | 97.8 | (0.274, 0.672) | 118 |
| Example 36 | 1-36 | 4.56 | 96.3 | (0.278, 0.678) | 117 |
| Example 37 | 1-37 | 4.79 | 88.3 | (0.277, 0.674) | 96 |
| Example 38 | 1-38 | 4.74 | 87.2 | (0.279, 0.674) | 98 |
| Example 39 | 1-39 | 4.70 | 86.3 | (0.280, 0.676) | 96 |
| Example 40 | 1-40 | 4.71 | 89.3 | (0.278, 0.672) | 99 |
| Example 41 | 1-41 | 4.52 | 95.7 | (0.283, 0.687) | 113 |
| Example 42 | 1-45 | 4.54 | 94.8 | (0.286, 0.686) | 116 |
| Example 43 | 1-46 | 4.60 | 96.2 | (0.274, 0.678) | 115 |
| Example 44 | 1-49 | 4.69 | 93.7 | (0.279, 0.677) | 105 |
| Example 45 | 1-53 | 4.55 | 95.4 | (0.278, 0.677) | 112 |
| Example 46 | 1-55 | 4.57 | 94.7 | (0.276, 0.671) | 116 |
| Example 47 | 1-56 | 4.59 | 94.7 | (0.286, 0.692) | 115 |
| Example 48 | 1-61 | 4.56 | 96.3 | (0.274, 0.672) | 117 |
| Example 49 | 1-67 | 4.60 | 96.6 | (0.278, 0.678) | 114 |
| Example 50 | 1-71 | 4.79 | 88.3 | (0.279, 0.677) | 98 |
| Example 51 | 1-72 | 4.77 | 90.2 | (0.277, 0.674) | 99 |
| Example 52 | 1-73 | 4.55 | 97.2 | (0.279, 0.674) | 113 |
| Example 53 | 1-75 | 4.59 | 97.0 | (0.278, 0.677) | 119 |
| Example 54 | 1-77 | 4.76 | 90.3 | (0.279, 0.681) | 100 |
| Example 55 | 1-78 | 4.70 | 89.7 | (0.280, 0.679) | 99 |
| Example 56 | 1-81 | 4.58 | 97.2 | (0.281, 0.678) | 118 |
| Example 57 | 1-82 | 4.62 | 93.5 | (0.278, 0.682) | 108 |
| Example 58 | 1-85 | 4.57 | 96.8 | (0.286, 0.692) | 118 |
| Example 59 | 1-90 | 4.77 | 90.2 | (0.274, 0.672) | 95 |
| Example 60 | 1-92 | 4.75 | 89.7 | (0.279, 0.681) | 98 |
| Example 61 | 1-101 | 4.55 | 98.7 | (0.277, 0.674) | 117 |
| Example 62 | 1-109 | 4.82 | 88.3 | (0.279, 0.674) | 89 |
| Example 63 | 1-113 | 4.60 | 97.8 | (0.281, 0.678) | 120 |
| Example 64 | 1-116 | 4.58 | 98.9 | (0.278, 0.682) | 118 |
| Example 65 | 1-120 | 4.79 | 88.2 | (0.286, 0.692) | 98 |
| Example 66 | 1-123 | 4.68 | 92.7 | (0.274, 0.672) | 109 |
| Example 67 | 1-125 | 4.52 | 95.6 | (0.278, 0.678) | 114 |
| Example 68 | 1-128 | 4.57 | 99.8 | (0.277, 0.674) | 115 |
| Example 69 | 1-132 | 4.78 | 90.2 | (0.278, 0.682) | 90 |
| Example 70 | 1-138 | 4.59 | 95.4 | (0.286, 0.692) | 113 |
| Example 71 | 1-141 | 4.50 | 95.5 | (0.276, 0.671) | 114 |
| Example 72 | 1-145 | 4.56 | 98.7 | (0.286, 0.692) | 114 |
| Example 73 | 1-149 | 4.77 | 89.9 | (0.274, 0.672) | 93 |
| Example 74 | 1-154 | 4.53 | 101.1 | (0.278, 0.678) | 115 |
| Example 75 | 1-160 | 4.79 | 88.3 | (0.279, 0.677) | 95 |
| Example 76 | 1-173 | 4.59 | 99.7 | (0.277, 0.674) | 112 |
| Example 77 | 1-174 | 4.59 | 99.2 | (0.279, 0.674) | 111 |
| Example 78 | 1-177 | 4.90 | 88.2 | (0.278, 0.677) | 93 |
| Example 79 | 1-180 | 4.81 | 89.7 | (0.279, 0.681) | 92 |
| Example 80 | 1-181 | 4.58 | 96.4 | (0.280, 0.679) | 113 |
| Example 81 | 1-185 | 4.57 | 99.7 | (0.276, 0.671) | 117 |
| Example 82 | 1-193 | 4.55 | 98.4 | (0.286, 0.692) | 115 |
| Example 83 | 1-201 | 4.62 | 91.7 | (0.278, 0.682) | 102 |
| Example 84 | 1-205 | 4.72 | 87.2 | (0.286, 0.692) | 108 |
| Example 85 | 1-210 | 4.69 | 93.2 | (0.274, 0.672) | 103 |
| Example 86 | 1-231 | 4.62 | 86.2 | (0.279, 0.681) | 106 |
| Example 87 | 1-240 | 4.58 | 92.3 | (0.277, 0.674) | 109 |
| Example 88 | 1-241 | 4.60 | 92.4 | (0.279, 0.674) | 110 |
| Example 89 | 1-252 | 4.78 | 92.6 | (0.281, 0.678) | 107 |
| Example 90 | 1-259 | 4.64 | 85.4 | (0.278, 0.682) | 103 |
| Example 91 | 1-281 | 4.64 | 85.3 | (0.278, 0.682) | 99 |
| Example 92 | 1-282 | 4.60 | 85.4 | (0.286, 0.692) | 97 |
| Example 93 | 1-289 | 4.59 | 86.2 | (0.274, 0.672) | 97 |
| Example 94 | 1-294 | 4.58 | 81.7 | (0.278, 0.678) | 95 |
| Example 95 | 1-301 | 4.59 | 78.9 | (0.277, 0.674) | 98 |
| Example 96 | 1-302 | 4.71 | 81.2 | (0.278, 0.682) | 92 |
| Example 97 | 1-305 | 4.72 | 86.3 | (0.286, 0.692) | 93 |
| Example 98 | 1-310 | 4.75 | 85.5 | (0.274, 0.672) | 95 |
| Example 99 | 1-311 | 4.70 | 88.3 | (0.278, 0.678) | 93 |
| Example 100 | 1-318 | 4.71 | 90.3 | (0.277, 0.674) | 197 |

Referring to the results in Table 13, it can be seen that the organic light emitting device including a heterocyclic compound of the present application is superior to the Comparative Examples in terms of driving voltage, luminous efficiency, and lifetime. Particularly, it can be confirmed that the higher the deuterium substitution rate, the lower the driving voltage, resulting in excellent lifetime characteristics.

Specifically, the present application using a compound in which deuterium is included only at a specific position provides an organic light emitting device with a lower driving voltage, high luminous efficiency and a longer lifetime, compared to a comparative example using a compound that does not include deuterium. This is because when the compound of the present application is substituted with deuterium, which has a higher molecular weight than hydrogen, the change in vibrational frequency is reduced and the energy of the molecule was lowered, thereby increasing the stability of the molecule. In addition, since the single bond dissociation energy of carbon and deuterium is higher than that of carbon and hydrogen, it can be confirmed that the lifetime of the device is improved with the increased thermal stability of the molecule.

When the compound including deuterium only at a specific position as in Formula 1 according to the present application was deposited on a silicon wafer, the material including deuterium tends to be packed with a narrower intermolecular distance. In addition, when a thin film surface was observed by an atomic force microscope (AFM), it can be confirmed that the thin film made of the compound including deuterium is deposited with a more uniform surface without aggregation.

In addition, a molecule is thermally damaged by electron transfer during the operation of the organic light emitting device. Particularly, there is high possibility of defects at oxygen or sulfur, which is the most unstable site of Formula 1 of the present application. To prevent this, in the compound corresponding to Formula 1 of the present application, by substituting deuterium, which has a higher molecular weight than hydrogen, in the core structure of the heterocyclic compound, it can be confirmed that the change in vibrational frequency was reduced to lower molecular energy, thereby increasing the stability of the molecule and greatly increasing the lifetime of the device, compared to Comparative Examples.

In other words, when the heterocyclic compound of Formula 1 of the present application is used as a host of the emissive layer, it can be confirmed that it exhibited significantly excellent driving voltage, luminous efficiency and lifetime.

That is, it can be seen that the heterocyclic compound of the present application is a compound in which phenyls of naphthobenzofuran, naphthobenzothiophene, and dibenzofuran, dibenzothiophene, connected to triazine, are substituted, and has excellent thermal stability and luminous efficiency.

The compound of the present application, including naphthobenzofuran, has a shallow HOMO level and a narrow band gap. Due to such characteristics, it can be seen that it is effective in increasing lifetime by strengthening the hole injection characteristic in device evaluation. In addition, steric hindrance occurs between naphthobenzofuran, dibenzofuran, and triazine, resulting in improving driving and efficiency in device evaluation.

In addition, it was confirmed that the efficiency and lifetime are improved because in the case of the compound of the present application, hole transfer occurs close to the center of an EML layer in a recombination area due to deuterium being substituted in naphthobenzofuran, which is responsible for HOMO, compared to a compound in which deuterium is not substituted.

In addition, as aryl groups were substituted in naphthobenzofuran and dibenzofuran, molecular stability and thermal stability were increased due to the steric hindrance in the compound, confirming that the durability and stability of the device are improved, resulting in the extended lifetime of the device.

### Experimental Example 2

1) Manufacture of Organic Light Emitting Device

A glass substrate on which an ITO thin film was coated to a thickness of 1,500 Å was cleaned with ultrasonic waves in distilled water. After distilled water cleaning, ultrasonic cleaning was performed with a solvent such as acetone, methanol, or isopropyl alcohol followed by drying, and then UVO treatment was performed in an UV cleaner for 5 minutes using UV. Afterward, the substrate was transferred to a plasma cleaner (PT), subjected to plasma treatment to remove the ITP work function and a residual film and then transferred to thermal deposition equipment for organic deposition.

On the ITO transparent electrode (positive electrode), common layers, such as a hole injection layer (4,4′,4″-Tris[2-naphthyl(phenyl)amino]triphenylamine (2-TNATA)) and a hole transport layer (N,N′-Di(1-naphthyl)-N,N′-diphenyl-(1,1′-biphenyl)-4,4′-diamine (NPB), were formed.

An emissive layer was formed thereon through thermal vacuum deposition as follows. The emissive layer was deposited to 400 Å in one source after pre-mixing of one type of heterocyclic compound of Formula 1 and one type of compound of Formula 2 as hosts, and a green phosphorescent dopant was deposited by doping Ir(ppy)₃ at 7% the deposition thickness of the emissive layer. Afterward, as a hole blocking layer, 60 Å of BCP was deposited thereon, and as an electron transport layer, 200 Å of Alq₃ was deposited thereon. Finally, lithium fluoride (LiF) was deposited to 10 Å on the electron transport layer, thereby forming an electron injection layer, and on the electron injection layer, an aluminum (Al) negative electrode was deposited to 1,200 Å, thereby forming a negative electrode, resulting in the manufacture of an organic electroluminescent device.

In Table 14, Examples and Comparative Examples other than those separately denoted as red hosts were used as green hosts.

In the case of Examples used as red hosts, the organic electroluminescent device was manufactured in the same manner as described above, except that Ir(piq)₂(acac) was used as a red phosphorescent dopant, instead of a green phosphorescent dopant, to form an emissive layer, and bathophenanthroline (BPhen) was deposited to 30 Å, instead of BCP, to form a hole blocking layer.

Meanwhile, all organic compounds required for manufacturing an OLED were used in the manufacture of an OLED after purification of each material by vacuum sublimination under 10⁻⁸ to 10⁻⁶ torr.

Electroluminescent (EL) characteristics of electroluminescent device manufactured as described above were measured on the organic using M7000 (McScience), and with the measurement results, T₉₀ was measured when the standard luminance was 6,000 cd/m² using lifetime measurement equipment (M6000, MEScience).

The results of measuring the driving voltage, luminous efficiency, color coordinates (CIE), and lifetime of the organic light emitting device manufactured according to the present application are shown in Table 14 below.

TABLE 14

| | Emissive | Ratio | Driving | | | |
|---|---|---|---|---|---|---|
| | layer | (Weight | voltage | Efficiency | CIE | Lifetime |
| | compound | Ratio) | (V) | (cd/A) | (x, y) | (T90) |
| comparative | A:2-82 | 1:3 | 4.78 | 110.3 | (0.277, 0.682) | 206 |
| example 1 | | | | | | |
| comparative | | 1:2 | 4.72 | 111.7 | (0.277, 0.678) | 204 |
| example 2 | | | | | | |
| comparative | B:2-82 | 1:3 | 4.67 | 102.8 | (0.276, 0.679) | 208 |
| example 3 | | | | | | |
| comparative | | 1:2 | 4.64 | 108.7 | (0.277, 0.677) | 206 |
| example 4 | | | | | | |
| comparative | C:2-85 | 1:3.5 | 4.68 | 103.8 | (0.277, 0.680) | 207 |
| example 5 | | | | | | |
| comparative | | 1:2.5 | 4.66 | 104.9 | (0.278, 0.681) | 200 |
| example 6 | | | | | | |
| comparative | | 1:2 | 4.65 | 104.9 | (0.278, 0.681) | 200 |
| example 7 | | | | | | |
| comparative | D:2-86 | 1:3 | 4.67 | 102.3 | (0.277, 0.682) | 206 |
| example 8 | | | | | | |
| comparative | | 1:2 | 4.67 | 105.2 | (0.278, 0.683) | 205 |
| example 9 | | | | | | |
| comparative | E:2-84 | 1:3 | 4.54 | 135.2 | (0.277, 0.682) | 224 |
| example 10 | | | | | | |
| comparative | | 1:2 | 4.50 | 136.6 | (0.277, 0.678) | 222 |
| example 11 | | | | | | |
| comparative | F:2-86 | 1:3.5 | 4.49 | 136.9 | (0.276, 0.679) | 239 |
| example 12 | | | | | | |
| comparative | | 1:2.5 | 4.47 | 138.9 | (0.277, 0.677) | 236 |
| example 13 | | | | | | |
| comparative | | 1:2 | 4.40 | 141.1 | (0.277, 0.680) | 229 |
| example 14 | | | | | | |
| example A | 1-1:2-32 | 1:3.5 | 4.22 | 167.8 | (0.277, 0.677) | 297 |
| example B | | 1:3 | 4.20 | 170.2 | (0.277, 0.680) | 295 |
| example 1 | | 1:2.5 | 4.20 | 170.9 | (0.278, 0.681) | 294 |
| example 2 | | 1:2 | 4.18 | 171.1 | (0.278, 0.681) | 292 |
| example 3 | 1-1:2-47 | 1:3 | 3.85 | 172.3 | (0.277, 0.682) | 304 |
| example 4 | | 1:2.5 | 3.83 | 174.4 | (0.278, 0.683) | 302 |
| example 5 | | 1:2 | 3.82 | 175.2 | (0.277, 0.682) | 300 |
| example 6 | 1-1:2-78 | 1.3.5 | 3.79 | 175.3 | (0.277, 0.678) | 308 |
| example 7 | | 1:3 | 3.76 | 177.2 | (0.276, 0.679) | 307 |
| example 8 | | 1:2.5 | 3.70 | 177.9 | (0.277, 0.677) | 304 |
| example 9 | 1-1:2-89 | 1:3.5 | 3.75 | 176.6 | (0.277, 0.680) | 310 |
| example 10 | | 1:3 | 3.70 | 178.4 | (0.278, 0.681) | 308 |
| example 11 | | 1:2 | 3.68 | 178.8 | (0.277, 0.678) | 304 |
| example 12 | | 1:1 | 3.66 | 179.2 | (0.276, 0.679) | 303 |
| example 13 | 1-2:2-82 | 1:3 | 3.66 | 172.3 | (0.277, 0.677) | 307 |
| example 14 | | 1:2 | 3.59 | 178.8 | (0.277, 0.680) | 306 |
| example 15 | 1-4:2-86 | 1:3 | 3.67 | 173.4 | (0.278, 0.681) | 304 |
| example 16 | | 1:2 | 3.63 | 179.6 | (0.277, 0.682) | 300 |
| example 17 | 1-8:2-87 | 1:2.5 | 3.66 | 172.2 | (0.278, 0.683) | 310 |
| example 18 | | 1:1 | 3.60 | 177.3 | (0.277, 0.682) | 309 |
| example 19 | 1-9:2-25 | 1:3.5 | 4.19 | 160.3 | (0.278, 0.681) | 299 |
| example 20 | | 1:3 | 4.17 | 163.7 | (0.277, 0.682) | 290 |
| example 21 | | 1:2 | 4.09 | 166.7 | (0.278, 0.683) | 283 |
| example 22 | | 1:1 | 4.08 | 168.5 | (0.277, 0.682) | 280 |
| example 23 | 1-9:2-68 | 1:3 | 3.82 | 168.7 | (0.276, 0.679) | 309 |
| example 24 | | 1:2 | 3.80 | 169.2 | (0.277, 0.677) | 305 |
| example 25 | | 1:1 | 3.78 | 170.4 | (0.277, 0.680) | 304 |
| example 26 | 1-9:2-83 | 1:3.5 | 3.70 | 171.4 | (0.278, 0.683) | 310 |
| example 27 | | 1:2 | 3.66 | 173.2 | (0.277, 0.682) | 306 |
| example 28 | 1-12:2-88 | 1:3 | 3.71 | 172.3 | (0.277, 0.677) | 309 |
| example 29 | | 1:2 | 3.68 | 174.6 | (0.277, 0.680) | 304 |
| example 30 | | 1:1 | 3.64 | 177.9 | (0.278, 0.683) | 303 |
| example 31 | 1-16:2-43 | 1:3 | 3.88 | 169.7 | (0.278, 0.681) | 303 |
| example 32 | | 1:2 | 3.86 | 171.0 | (0.277, 0.682) | 301 |
| example 33 | | 1:1 | 3.80 | 171.9 | (0.278, 0.683) | 300 |
| example 34 | 1-16:2-80 | 1:3 | 3.66 | 174.3 | (0.277, 0.682) | 310 |
| example 35 | | 1:2 | 3.64 | 176.7 | (0.278, 0.681) | 306 |
| example 36 | | 1:1 | 3.60 | 179.4 | (0.277, 0.682) | 305 |
| example 37 | 1-16:2-89 | 1:3 | 3.61 | 176.5 | (0.278, 0.683) | 379 |
| example 38 | | 1:2 | 3.59 | 179.3 | (0.277, 0.682) | 370 |
| example 39 | | 1:1 | 3.58 | 179.9 | (0.276, 0.679) | 368 |
| example 40 | 1-18:2-87 | 1:3 | 3.60 | 186.2 | (0.277, 0.677) | 310 |
| example 41 | | 1:2 | 3.59 | 187.3 | (0.277, 0.680) | 308 |
| example 42 | 1-20:2-88 | 1:3 | 3.60 | 182.7 | (0.278, 0.683) | 307 |
| example 43 | | 1:2 | 3.59 | 186.3 | (0.277, 0.682) | 304 |
| example 44 | 1-23:2-87 | 1:3 | 3.68 | 176.3 | (0.277, 0.682) | 288 |
| example 45 | | 1:2 | 3.66 | 180.1 | (0.277, 0.678) | 285 |
| example 46 | 1-28:2-86 | 1:3 | 3.48 | 190.7 | (0.281, 0.676) | 375 |
| example 47 | | 1:2 | 3.40 | 194.4 | (0.280, 0.676) | 373 |
| example 48 | 1-29:2-81 | 1:3 | 3.66 | 178.2 | (0.276, 0.679) | 292 |
| example 49 | | 1:2 | 3.65 | 176.6 | (0.275, 0.679) | 290 |
| example 50 | 1-31:2-85 | 1:3 | 3.42 | 194.2 | (0.278, 0.678) | 377 |
| example 51 | | 1:2 | 3.39 | 196.3 | (0.279, 0.677) | 376 |
| example 52 | 1-34:2-4 | 1:3 | 3.98 | 148.2 | (0.275, 0.680) | 235 |
| example 53 | | 1:2 | 3.92 | 149.7 | (0.274, 0.678) | 230 |
| example 54 | 1-34:2-27 | 1:3 | 3.90 | 148.9 | (0.276, 0.676) | 250 |
| example 55 | | 1:2 | 3.85 | 150.0 | (0.274, 0.679) | 248 |
| example 56 | 1-34:2-32 | 1:3 | 3.91 | 147.7 | (0.288, 0.681) | 250 |
| example 57 | | 1:2 | 3.87 | 148.9 | (0.285, 0.680) | 249 |
| example 58 | 1-34:2-33 | 1:3 | 3.88 | 148.8 | (0.276, 0.679) | 248 |
| example 59 | | 1:2 | 3.85 | 149.2 | (0.276, 0.676) | 246 |
| example 60 | 1-34:2-42 | 1:3 | 3.92 | 152.6 | (0.278, 0.681) | 210 |
| example 61 | | 1:2.5 | 3.92 | 158.4 | (0.275, 0.680) | 207 |
| example 62 | | 1:2 | 3.90 | 150.3 | (0.274, 0.679) | 205 |
| example 63 | | 1:1 | 3.88 | 151.1 | (0.288, 0.681) | 203 |
| example 64 | 1-34:2-45 | 1:3 | 3.96 | 159.4 | (0.285, 0.680) | 230 |
| example 65 | | 1:2 | 3.94 | 151.1 | (0.276, 0.679) | 226 |
| example 66 | | 1:1.5 | 3.85 | 156.3 | (0.276, 0.676) | 220 |
| example 67 | | 1:1 | 3.85 | 157.6 | (0.278, 0.681) | 200 |
| example 68 | 1-34:2-65 | 1:3 | 3.80 | 163.7 | (0.274, 0.679) | 300 |
| example 69 | | 1:2 | 3.78 | 165.5 | (0.288, 0.681) | 296 |
| example 70 | | 1:1.5 | 3.77 | 166.7 | (0.285, 0.680) | 295 |
| example 71 | | 1:1 | 3.74 | 168.9 | (0.276, 0.679) | 290 |
| example 72 | 1-34:2-69 | 1:3 | 3.78 | 164.2 | (0.274, 0.679) | 298 |
| example 73 | | 1:2.5 | 3.76 | 166.4 | (0.288, 0.681) | 295 |
| example 74 | | 1:2 | 3.75 | 167.9 | (0.285, 0.680) | 290 |
| example 75 | 1-34:2-74 | 1:2 | 3.79 | 167.3 | (0.276, 0.679) | 292 |
| example 76 | | 1:1.5 | 3.76 | 169.2 | (0.280, 0.676) | 290 |
| example 77 | | 1:1 | 3.75 | 170.0 | (0.276, 0.679) | 288 |
| example 78 | 1-34:2-82 | 1:3.5 | 3.74 | 171.1 | (0.274, 0.678) | 310 |
| example 79 | | 1:2.5 | 3.72 | 175.6 | (0.276, 0.676) | 308 |
| example 80 | | 1:2 | 3.63 | 177.3 | (0.274, 0.679) | 305 |
| example 81 | | 1:1 | 3.60 | 180.4 | (0.288, 0.681) | 304 |
| example 82 | 1-34:2-83 | 1:3 | 3.62 | 173.2 | (0.280, 0.676) | 309 |
| example 83 | | 1:2 | 3.61 | 178.3 | (0.276, 0.679) | 308 |
| example 84 | 1-38:2-85 | 1:3 | 3.70 | 178.3 | (0.274, 0.679) | 312 |
| example 85 | | 1:2 | 3.68 | 180.7 | (0.288, 0.681) | 309 |
| example 86 | | 1:1 | 3.65 | 181.3 | (0.285, 0.680) | 306 |
| example 87 | 1-39:2-88 | 1:3 | 3.69 | 178.7 | (0.275, 0.680) | 310 |
| example 88 | | 1:2.5 | 3.62 | 180.3 | (0.274, 0.679) | 309 |
| example 89 | | 1:1 | 3.61 | 181.1 | (0.274, 0.679) | 306 |
| example 90 | 1-42:2-83 | 1:3.5 | 3.75 | 173.2 | (0.277, 0.678) | 310 |
| example 91 | | 1:2.5 | 3.70 | 178.3 | (0.276, 0.679) | 309 |
| example 92 | | 1:2 | 3.66 | 180.4 | (0.277, 0.682) | 304 |
| example 93 | 1-43:2-85 | 1:3 | 3.74 | 171.3 | (0.277, 0.678) | 308 |
| example 94 | | 1:2 | 3.70 | 176.1 | (0.276, 0.677) | 304 |
| example 95 | | 1:1.5 | 3.68 | 179.4 | (0.280, 0.679) | 303 |
| example 96 | 1-47:2-87 | 1:2 | 3.75 | 170.6 | (0.278, 0.681) | 310 |
| example 97 | | 1:1 | 3.65 | 179.3 | (0.276, 0.679) | 309 |
| example 98 | 1-49:2-98 | 1:3 | 3.70 | 170.4 | (0.277, 0.678) | 310 |
| example 99 | | 1:2 | 3.62 | 177.7 | (0.276, 0.677) | 305 |
| example 100 | 1-53:2-96 | 1:3 | 3.77 | 170.6 | (0.280, 0.679) | 308 |
| example 101 | | 1:2 | 3.65 | 174.8 | (0.278, 0.681) | 306 |
| example 102 | 1-57:2-88 | 1:3 | 3.71 | 170.3 | (0.276, 0.677) | 310 |
| example 103 | | 1:2.5 | 3.68 | 173.7 | (0.280, 0.679) | 308 |
| example 104 | | 1:2 | 3.65 | 175.4 | (0.278, 0.681) | 307 |
| example 105 | 1-60:2-89 | 1:3.5 | 3.68 | 170.7 | (0.277, 0.678) | 308 |
| example 106 | | 1:2.5 | 3.62 | 172.7. | (0.276, 0.677) | 305 |
| example 107 | | 1:2 | 3.60 | 173.7 | (0.280, 0.679) | 304 |
| example 108 | 1-62:2-80 | 1:3.5 | 3.73 | 170.3 | (0.276, 0.677) | 310 |
| example 109 | | 1:2 | 3.70 | 172.6 | (0.278, 0.681) | 304 |
| example 110 | | 1:1 | 3.65 | 175.3 | (0.276, 0.679) | 302 |
| example 111 | 1-63:2-86 | 1:3 | 3.70 | 171.1 | (0.276, 0.677) | 305 |
| example 112 | | 1:2.5 | 3.64 | 176.3 | (0.280, 0.679) | 303 |
| example 113 | 1-68:2-88 | 1:3.5 | 3.71 | 170.4 | (0.277, 0.678) | 310 |
| example 114 | | 1:2 | 3.68 | 173.1 | (0.276, 0.677) | 306 |
| example 115 | | 1:1 | 3.62 | 174.3 | (0.280, 0.679) | 303 |
| example 116 | 1-69:2-82 | 1:3.5 | 3.60 | 182.3 | (0.276, 0.677) | 310 |
| example 117 | | 1:2.5 | 3.59 | 188.3 | (0.280, 0.679) | 309 |
| example 118 | | 1:2 | 3.56 | 190.1 | (0.278, 0.681) | 308 |
| example 119 | | 1:1 | 3.55 | 190.7 | (0.278, 0.681) | 303 |
| example 120 | 1-71:2-82 | 1:3.5 | 3.70 | 174.2 | (0.276, 0.677) | 300 |
| example 121 | | 1:2.5 | 3.66 | 177.9 | (0.280, 0.679) | 299 |
| example 122 | | 1:2 | 3.65 | 179.2 | (0.278, 0.681) | 294 |
| example 123 | | 1:1 | 3.60 | 180.3 | (0.276, 0.677) | 290 |
| example 124 | 1-72:2-16 | 1:3 | 3.62 | 176.6 | (0.280, 0.679) | 300 |
| example 125 | | 1:2 | 3.60 | 178.4 | (0.276, 0.677) | 292 |
| example 126 | 1-72:2-33 | 1:3 | 3.61 | 177.9 | (0.278, 0.681) | 299 |
| example 127 | | 1:2 | 3.60 | 179.9 | (0.276, 0.679) | 296 |
| example 128 | 1-72:2-42 | 1:3.5 | 3.61 | 183.2 | (0.276, 0.677) | 320 |
| example 129 | | 1:3 | 3.56 | 185.1 | (0.280, 0.679) | 315 |
| example 130 | | 1:2 | 3.55 | 185.5 | (0.277, 0.678) | 314 |
| example 131 | 1-72:2-66 | 1:3 | 3.53 | 187.3 | (0.276, 0.677) | 340 |
| example 132 | | 1:2 | 3.50 | 189.2 | (0.280, 0.679) | 338 |
| example 133 | 1-72:2-74 | 1:3 | 3.52 | 188.3 | (0.280, 0.679) | 339 |
| example 134 | | 1:2 | 3.50 | 190.1 | (0.276, 0.677) | 336 |
| example 135 | 1-72:2-82 | 1:3.5 | 3.47 | 191.3 | (0.277, 0.678) | 380 |
| example 136 | | 1:2.5 | 3.45 | 194.7 | (0.276, 0.677) | 378 |
| example 137 | | 1:2 | 3.41 | 199.3 | (0.280, 0.679) | 377 |
| example 138 | | 1:1 | 3.39 | 200.7 | (0.280, 0.679) | 370 |
| example 139 | 1-74:2-82 | 1:3.5 | 3.60 | 188.3 | (0.274, 0.679) | 311 |
| example 140 | | 1:2.5 | 3.59 | 189.7 | (0.288, 0.681) | 308 |
| example 141 | | 1:2 | 3.57 | 190.3 | (0.285, 0.680) | 307 |
| example 142 | | 1:1 | 3.55 | 192.3 | (0.285, 0.680) | 304 |
| example 143 | 1-81:2-82 | 1:3.5 | 3.50 | 194.4 | (0.276, 0.677) | 380 |
| example 144 | | 1:3 | 3.48 | 197.2 | (0.278, 0.681) | 374 |
| example 145 | | 1:2.5 | 3.45 | 198.3 | (0.276, 0.679) | 370 |
| example 146 | | 1:2 | 3.40 | 199.0 | (0.274, 0.679) | 369 |
| example 147 | | 1:1 | 3.30 | 199.8 | (0.288, 0.681) | 368 |
| example 148 | 1-81:2-81 | 1:3 | 3.66 | 178.3 | (0.277, 0.678) | 310 |
| example 149 | | 1:2 | 3.64 | 180.2 | (0.276, 0.677) | 308 |
| example 150 | 1-81:2-84 | 1:2.5 | 3.63 | 177.6 | (0.280, 0.679) | 309 |
| example 151 | | 1:2 | 3.62 | 178.2 | (0.280, 0.679) | 308 |
| example 152 | | 1:1 | 3.60 | 179.3 | (0.274, 0.679) | 307 |
| example 153 | 1-92:2-82 | 1:3 | 3.70 | 176.3 | (0.288, 0.681) | 310 |
| example 154 | | 1:2.5 | 3.66 | 178.9 | (0.285, 0.680) | 309 |
| example 155 | | 1:1 | 3.62 | 180.2 | (0.285, 0.680) | 308 |
| example 156 | 1-94:2-81 | 1:3 | 3.68 | 176.3 | (0.277, 0.678) | 309 |
| example 157 | | 1:2 | 3.64 | 177.7 | (0.276, 0.677) | 304 |
| example 158 | 1-95:2-83 | 1:2.5 | 3.69 | 177.7 | (0.280, 0.679) | 309 |
| example 159 | | 1:2 | 3.64 | 179.3 | (0.280, 0.679) | 308 |
| example 160 | | 1:1 | 3.60 | 180.4 | (0.274, 0.679) | 306 |
| example 161 | 1-102:2-84 | 1:3 | 3.62 | 174.5 | (0.288, 0.681) | 309 |
| example 162 | | 1:2 | 3.60 | 177.7 | (0.285, 0.680) | 304 |
| example 163 | 1-108:2-81 | 1:3 | 3.58 | 188.7 | (0.285, 0.680) | 310 |
| example 164 | | 1:2 | 3.56 | 190.2 | (0.277, 0.678) | 309 |
| example 165 | 1-118:2-83 | 1:3 | 3.42 | 194.9 | (0.276, 0.677) | 380 |
| example 166 | | 1:2 | 3.40 | 199.2 | (0.280, 0.679) | 377 |
| example 167 | 1-125:2-91 | 1:3 | 3.40 | 196.3 | (0.280, 0.679) | 379 |
| example 168 | | 1:2 | 3.32 | 199.4 | (0.274, 0.679) | 377 |
| example 169 | 1-129:2-97 | 1:3 | 3.70 | 178.4 | (0.288, 0.681) | 310 |
| example 170 | | 1:2 | 3.68 | 179.9 | (0.285, 0.680) | 308 |
| example 171 | 1-143:2-91 | 1:3 | 3.68 | 176.4 | (0.285, 0.680) | 309 |
| example 172 | | 1:2.5 | 3.62 | 177.2 | (0.277, 0.678) | 307 |
| example 173 | | 1:2 | 3.60 | 179.5 | (0.276, 0.677) | 306 |
| example 174 | 1-145:2-91 | 1:3 | 3.64 | 178.4 | (0.280, 0.679) | 310 |
| example 175 | | 1:2.5 | 3.62 | 179.0 | (0.280, 0.679) | 307 |
| example 176 | | 1:2 | 3.60 | 170.1 | (0.274, 0.679) | 306 |
| example 177 | 1-149:2-88 | 1:3 | 3.60 | 181.4 | (0.288, 0.681) | 310 |
| example 178 | | 1:2 | 3.59 | 188.7 | (0.285, 0.680) | 309 |
| example 179 | 1-153:2-97 | 1:3 | 3.64 | 179.2 | (0.285, 0.680) | 304 |
| example 180 | | 1:2 | 3.62 | 180.8 | (0.277, 0.678) | 302 |
| example 181 | 1-281:2-32 | 1:3.5 | 4.22 | 167.8 | (0.277, 0.677) | 297 |
| example 182 | | 1:3 | 4.20 | 170.2 | (0.277, 0.680) | 295 |
| example 183 | | 1:2.5 | 4.20 | 170.9 | (0.278, 0.681) | 294 |
| example 184 | | 1:2 | 4.18 | 171.1 | (0.278, 0.681) | 292 |
| example 185 | 1-285:2-47 | 1:3 | 3.85 | 172.3 | (0.277, 0.682) | 304 |
| example 186 | | 1:2.5 | 3.83 | 174.4 | (0.278, 0.683) | 302 |
| example 187 | | 1:2 | 3.82 | 175.2 | (0.277, 0.682) | 300 |
| example 188 | 1-289:2-78 | 1.3.5 | 3.79 | 175.3 | (0.277, 0.678) | 308 |
| example 189 | | 1:3 | 3.76 | 177.2 | (0.276, 0.679) | 307 |
| example 190 | | 1:2.5 | 3.70 | 177.9 | (0.277, 0.677) | 304 |
| example 191 | 1-318:2-89 | 1:3.5 | 3.75 | 176.6 | (0.277, 0.680) | 310 |
| example 192 | | 1:3 | 3.70 | 178.4 | (0.278, 0.681) | 308 |
| example 193 | | 1:2 | 3.68 | 178.8 | (0.277, 0.678) | 304 |
| example 194 | | 1:1 | 3.66 | 179.2 | (0.276, 0.679) | 303 |

Comparing the results in Table 14 and the results in Table 13, when the heterocyclic compound of Formula 1 and the compound of Formula 2 were used as hosts of the emissive layer at the same time, it can be confirmed that the lifetime is improved by approximately 3 times, and the driving voltage and luminous efficiency were improved by approximately 40% and 50%, respectively.

On the other hand, when a compound not included in the scope of the present application is used in combination with the compound of Formula 2, it can be seen that the lifetime is similar and the performance in terms of driving voltage and luminous efficiency was reduced, compared to when the heterocyclic compound of the present application was used alone.

That is, when the heterocyclic compound of Formula 1 and the compound of Formula 2 according to the present application were simultaneously used as hosts of the emissive layer, it can be confirmed that the driving voltage, luminous efficiency and lifetime are remarkably excellent.

Particularly, comparing Examples which use the same heterocyclic compound of Formula 1 and the compound of Formula 2 with the same structure, and differ only in whether or not deuterium is substituted, when the compound of Formula 2 is substituted with deuterium, it can be confirmed that the driving voltage, luminous efficiency and lifetime are excellent.

This is because, when the compound of Formula 2 also includes deuterium, as well as the heterocyclic compound of Formula 1, the compound of Formula 2 is also more effective in improving the performance of the organic light emitting device as molecular stability and thermal stability increase.

In addition, a device including a combination of Formulas 1 and 2 shows optimal driving, efficiency and lifetime results when the combination ratio of an N-type host and a P-type host is particularly 1:3. This is the result of the tendency for driving and lifetime to increase and efficiency to decrease as the proportion of the N-type host decreases. Particularly, when the proportion of the N-type host is less than 23%, that is, below 1:4, the driving and lifetime tend to be similar or gradually increase. On the other hand, it can be confirmed through experiments that the efficiency rapidly decreased. Accordingly, particularly, in the case of a device manufactured by combining the compounds of the present application, when the proportion of the N type host is 30% or more, good results may be obtained in terms of driving, efficiency and lifetime. Accordingly, it was confirmed that the compound of Formula 1 plays an important role in increasing the efficiency of the device.

That is, when the compound of Formula 1 and the compound of Formula 2 are simultaneously included, much better efficiency and lifetime effects are exhibited. This result can be expected because, when both compounds are included, an exciplex phenomenon occurs.

The exciplex phenomenon is a phenomenon in which energy having magnitudes of the HOMO level of a donor (p-host) and the LUMO level of an acceptor (n-host) is emitted due to the electron exchange between two molecules. When the exciplex phenomenon between two molecules occurs, reverse intersystem crossing (RISC) occurs, and thus the internal quantum efficiency of fluorescence may increase up to 100%. When a donor (p-post) having good hole transport ability and an acceptor (n-host) having good electron transport ability are used as hosts of the emissive layer, since holes are injected into the p-host and electrons are injected into the n-host, a driving voltage may be lowered, thereby helping to improve the lifetime. In the present application, when the compound of Formula 2, serving as a donor, and the compound of Formula 1, serving as an acceptor, were used as hosts of the emissive layer, it can be confirmed that excellent device characteristics are exhibited.

When a compound represented by Formula 2 that includes deuterium is used in the device, compared to when a compound not including deuterium is used, it can be confirmed that the device exhibited excellent performance.

More specifically, among all cases in which Formula 2 of the present application is partially substituted with deuterium, compared to when the hydrogen in each of the aryl groups corresponding to R21 and R22 of Formula 2 is substituted with deuterium, it can be confirmed that, when the hydrogen of biscarbazole is substituted with deuterium, the device exhibited much better performance, and as the deuterium content increased, the device exhibited better performance.

When a heterocyclic compound disclosed in the present specification is used in an organic light emitting device, it is possible to lower the driving voltage of the device, improve light efficiency, and improve the lifetime characteristics of the device. Particularly, since the heterocyclic compound of Formula 1 necessarily includes deuterium at the position of the core structure

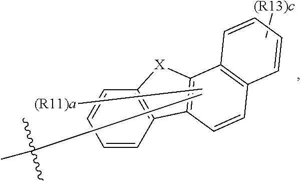

when used as a material for an emissive layer of the organic light emitting device, the stability of the molecule is increased by reducing the change in vibration frequency, and thermal stability is increased due to high single bond dissociation energy, providing excellent performance in terms of driving voltage, light emitting efficiency, and lifetime.

It will be apparent to those skilled in the art that various modifications can be made to the above-described exemplary embodiments of the present application without departing from the spirit or scope of the invention. Thus, it is intended that the present application covers all such modifications provided they come within the scope of the appended claims and their equivalents.

### EXPLANATION OF REFERENCE NUMERALS

100: Substrate
200: Positive electrode
300: Organic material layer
301: Hole injection layer
302: Hole transport layer
303: Emissive layer
304: Hole blocking layer
305: Electron transport layer
306: Electron injection layer
400: Negative electrode

1. A heterocyclic compound of Formula 1 below: 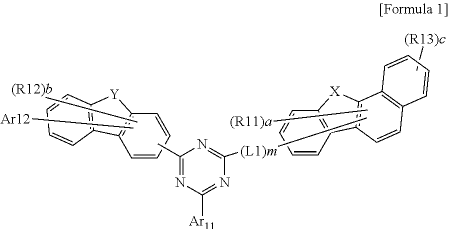
In Formula 1,
X and Y are the same or different, and are each independently O; S; or CRR′,
Ar11 and Ar12 are the same or different, and are each independently a substituted or unsubstituted C1 to C60 alkyl group; a substituted or unsubstituted C3 to C60 cycloalkyl group;
a substituted or unsubstituted C2 to C60 heterocycloalkyl group; a substituted or unsubstituted C6 to C60 aryl group; or a substituted or unsubstituted C2 to C60 heteroaryl group,
L1 is a direct bond; a substituted or unsubstituted C6 to C60 arylene group; or a substituted or unsubstituted C2 to C60 heteroarylene group,
R11 to R13 are the same or different, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R and R′ are the same or different, and are each independently a substituted or unsubstituted C1 to C60 alkyl group; a substituted or unsubstituted C6 to C60 aryl group; or a substituted or unsubstituted C2 to C60 heteroaryl group,
m and c are the same or different, and are each independently an integer of 0 to 4,
a is an integer of 0 to 5,
b is an integer of 0 to 6,
when a to c, and m are 2 or more, the substituents in parentheses are the same or different,
the deuterium content in 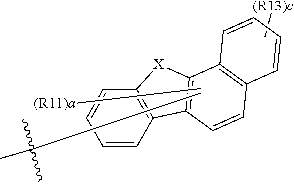
of Formula 1 is more than 0% and 100% or less,
the deuterium content of Formula 1 is less than 100%, and 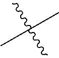
indicates the bonding position with Formula 1.

2. The heterocyclic compound of claim 1, wherein Formula 1 is represented by Formula 3 or 4 below: 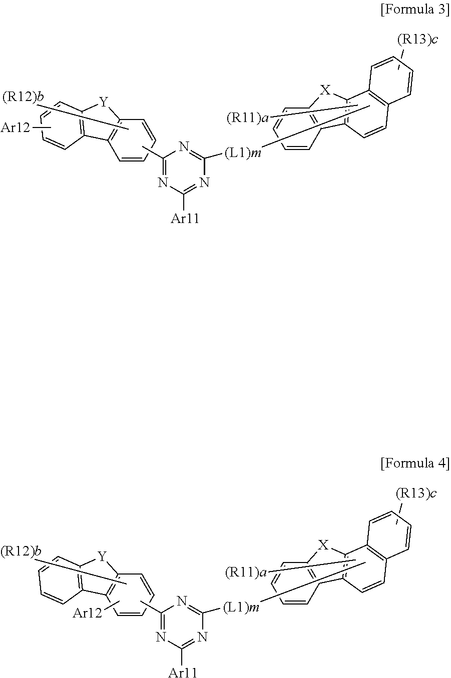
In Formulas 3 and 4,
the definition of each substituent is the same as that in Formula 1.

3. The heterocyclic compound of claim 2, wherein Formula 3 is represented by any one of Formulas 3-1 to 3-4 below: 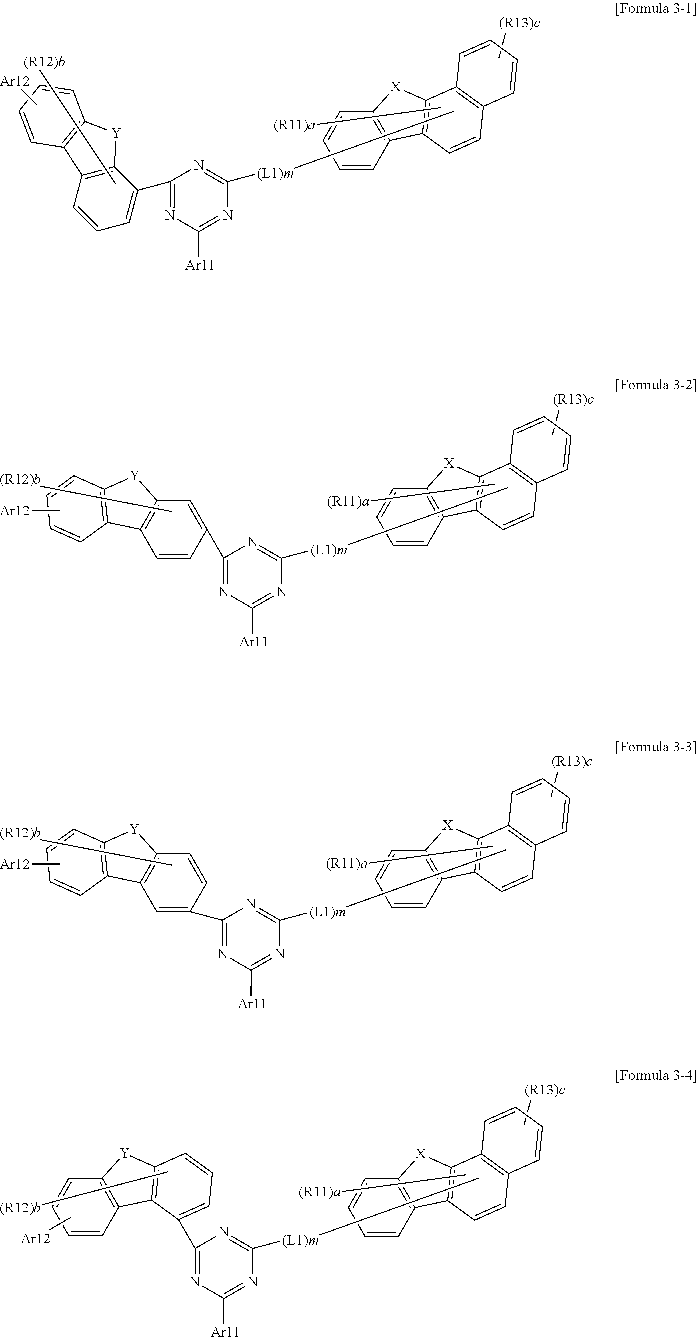
In Formulas 3-1 to 3-4,
the definition of each substituent is the same as that in Formula 3.

4. The heterocyclic compound of claim 2, wherein Formula 4 is represented by any one of Formulas 4-1 to 4-4: 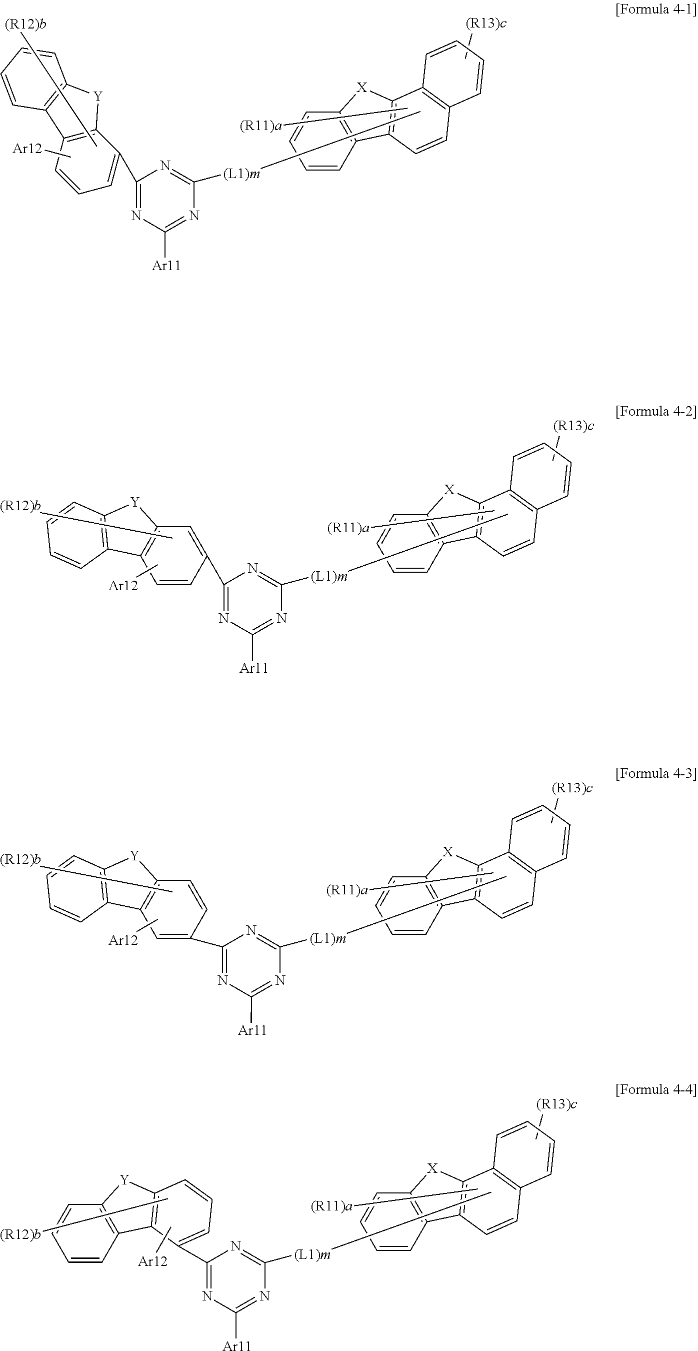
In Formulas 4-1 to 4-4,
the definition of each substituent is the same as that in Formula 4.

5. The heterocyclic compound of claim 1, wherein 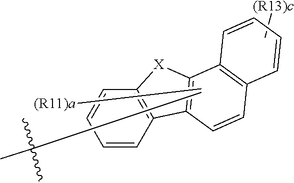
of Formula 1 is represented by any one of Formulas 1-1 to 1-6 below: 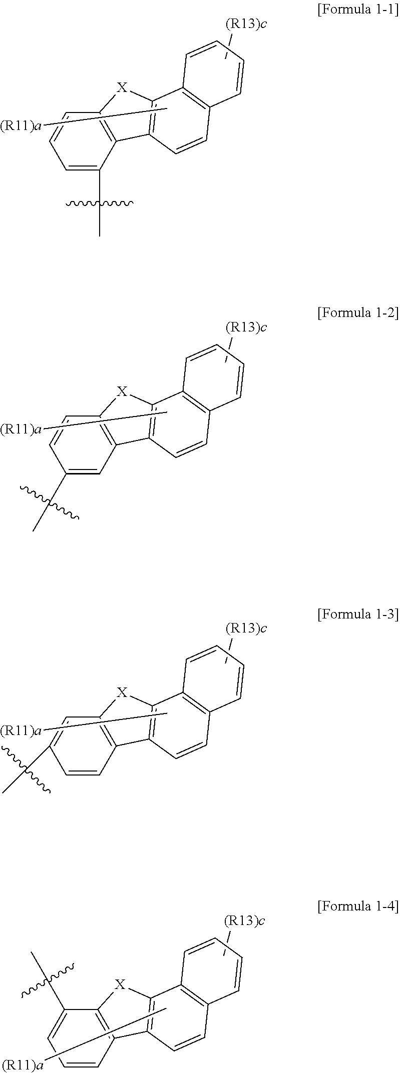 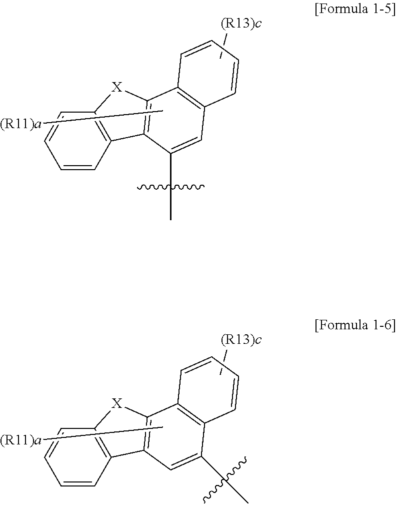
In Formulas 1-1 to 1-6
the definition of each substituent is the same as that in Formula 1.

6. The heterocyclic compound of claim 1, wherein Formula 1 comprises the structures of Formulas A to C below,
the deuterium content in Formula C is 50% X to 100%,
the deuterium contents in Formulas A and B below are 0% to 10%: 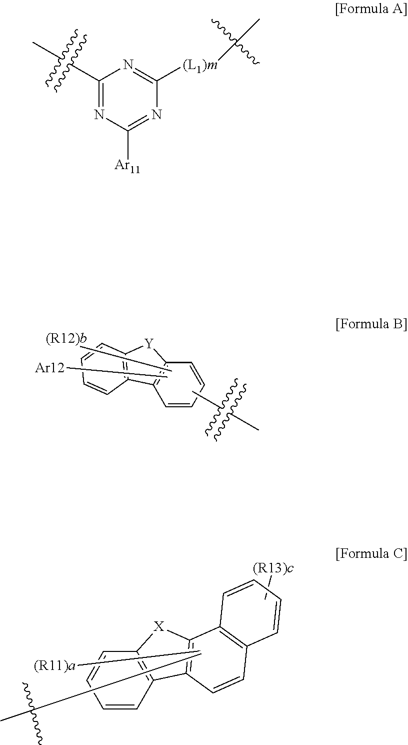
In Formulas A to C, 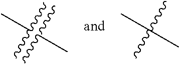
in the structures indicate the positions where the corresponding structure are bonded,
the definition of each substituent is the same as that in Formula 1.

7. The heterocyclic compound of claim 1, wherein the deuterium content of 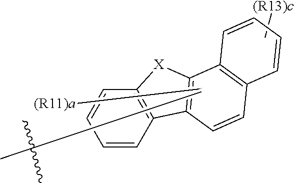
in Formula 1 is more than 50% and 100% or less.

8. The heterocyclic compound of claim 1, wherein 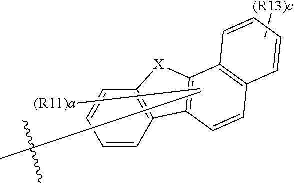
of Formula 1 is any one of Formulas A-1 to A-4 below: 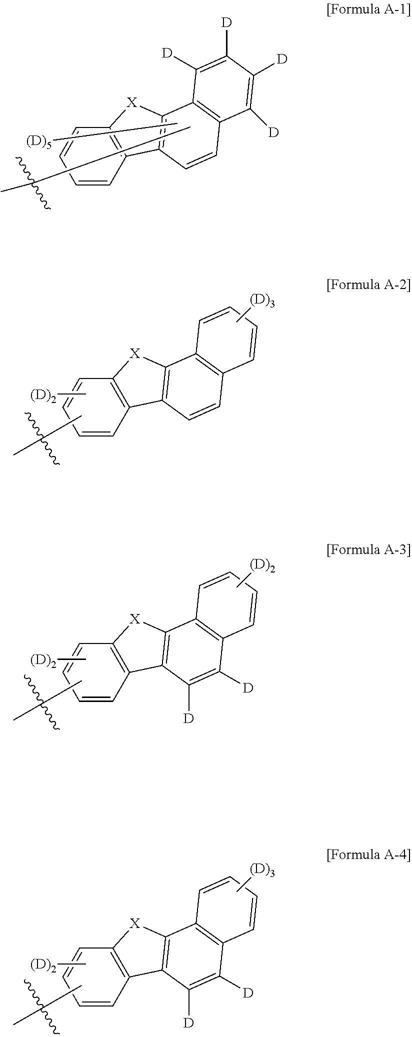
In Formulas A-1 to A-4, 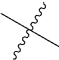
indicates the bonding position with Formula 1,
X is the same as the definition in Formula 1.

9. The heterocyclic compound of claim 1, wherein Formula 1 is represented by any one of the following compounds:

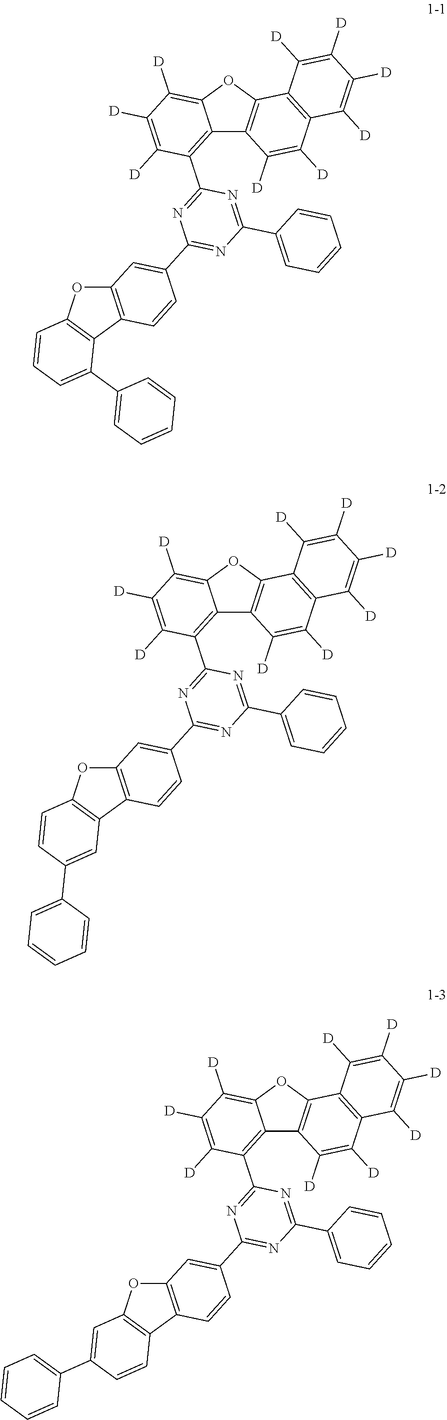 [SMILES: [2H]c1c([2H])c(-c2nc(-c3ccccc3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3ccc(-c5ccccc5)cc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3cc(-c5ccccc5)ccc34)n2)c2c(oc3c4c([2H])c([2H])c([2H])c([2H])c4c([2H])c([2H])c32)c1[2H]]

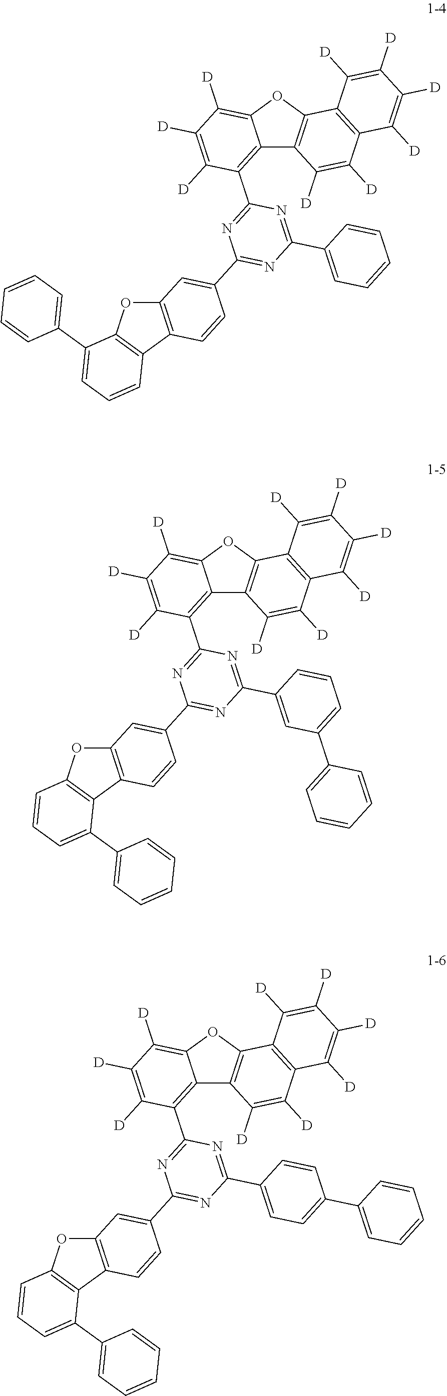 [SMILES: [2H]c1c([2H])c(-c2nc(-c3ccccc3)nc(-c3ccc4c(c3)oc3c(-c5ccccc5)cccc34)n2)c2c(oc3c4c([2H])c([2H])c([2H])c([2H])c4c([2H])c([2H])c32)c1[2H] ; [2H]c1c([2H])c(-c2nc(-c3cccc(-c4ccccc4)c3)nc(-c3ccc4c(c3)oc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccc(-c4ccccc4)cc3)nc(-c3ccc4c(c3)oc3cccc(-c5ccccc5)c34)n2)c2c(oc3c4c([2H])c([2H])c([2H])c([2H])c4c([2H])c([2H])c32)c1[2H]]

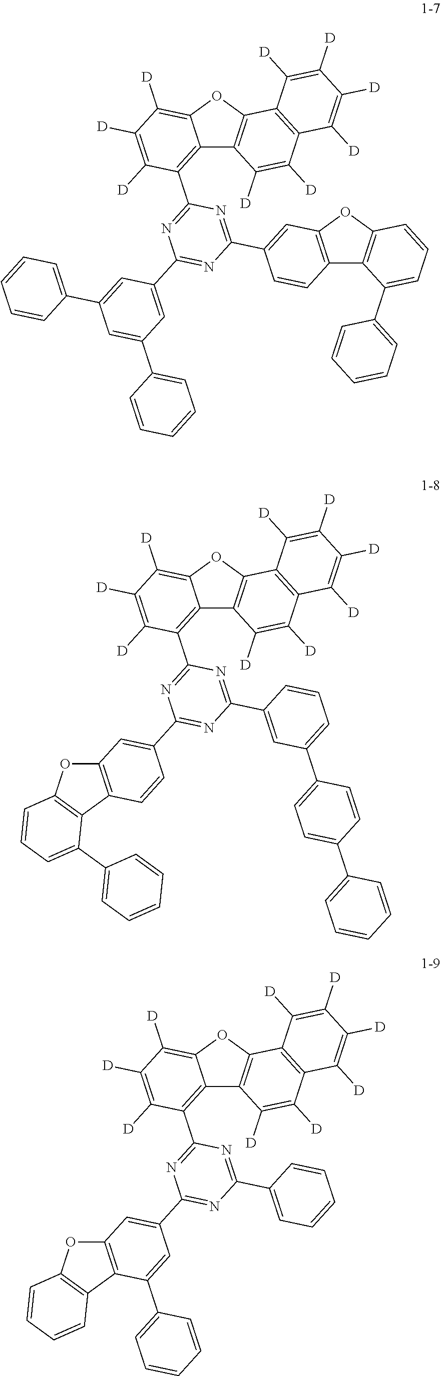

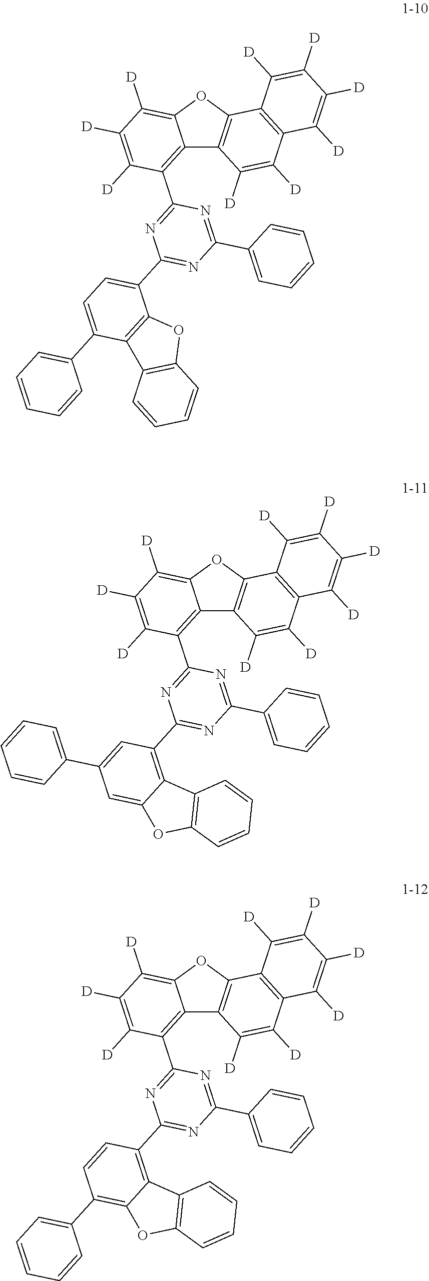 [SMILES: [2H]c1c([2H])c(-c2nc(-c3ccccc3)nc(-c3ccc(-c4ccccc4)c4c3oc3ccccc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3cc(-c4ccccc4)cc4oc5ccccc5c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc(-c4ccccc4)c4oc5ccccc5c34)n2)c2c(oc3c4c([2H])c([2H])c([2H])c([2H])c4c([2H])c([2H])c32)c1[2H]]

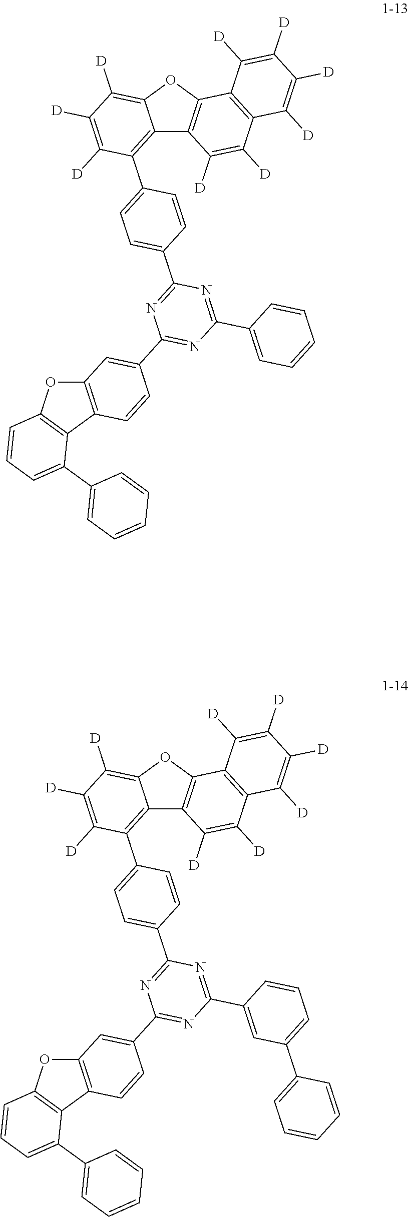 [SMILES: [2H]c1c([2H])c(-c2ccc(-c3nc(-c4ccccc4)nc(-c4ccc5c(c4)oc4cccc(-c6ccccc6)c45)n3)cc2)c2c(oc3c4c([2H])c([2H])c([2H])c([2H])c4c([2H])c([2H])c32)c1[2H] ; [2H]c1c([2H])c(-c2ccc(-c3nc(-c4cccc(-c5ccccc5)c4)nc(-c4ccc5c(c4)oc4cccc(-c6ccccc6)c45)n3)cc2)c2c(oc3c4c([2H])c([2H])c([2H])c([2H])c4c([2H])c([2H])c32)c1[2H]]

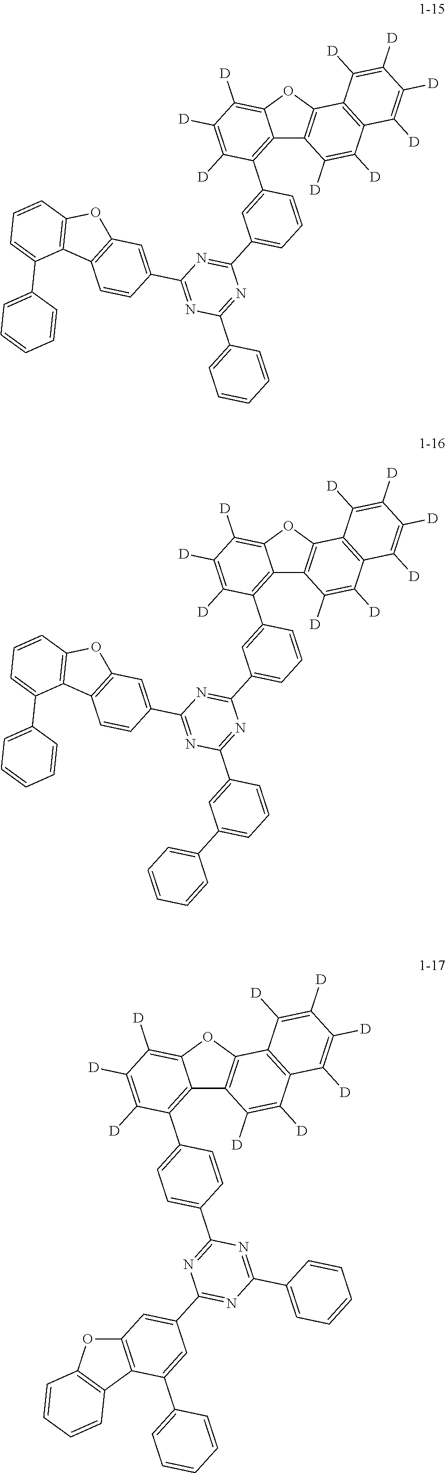 [SMILES: [2H]c1c([2H])c(-c2cccc(-c3nc(-c4ccccc4)nc(-c4ccc5c(c4)oc4cccc(-c6ccccc6)c45)n3)c2)c2c(oc3c4c([2H])c([2H])c([2H])c([2H])c4c([2H])c([2H])c32)c1[2H] ; [2H]c1c([2H])c(-c2cccc(-c3nc(-c4cccc(-c5ccccc5)c4)nc(-c4ccc5c(c4)oc4cccc(-c6ccccc6)c45)n3)c2)c2c(oc3c4c([2H])c([2H])c([2H])c([2H])c4c([2H])c([2H])c32)c1[2H] ; [2H]c1c([2H])c(-c2ccc(-c3nc(-c4ccccc4)nc(-c4cc(-c5ccccc5)c5c(c4)oc4ccccc45)n3)cc2)c2c(oc3c4c([2H])c([2H])c([2H])c([2H])c4c([2H])c([2H])c32)c1[2H]]

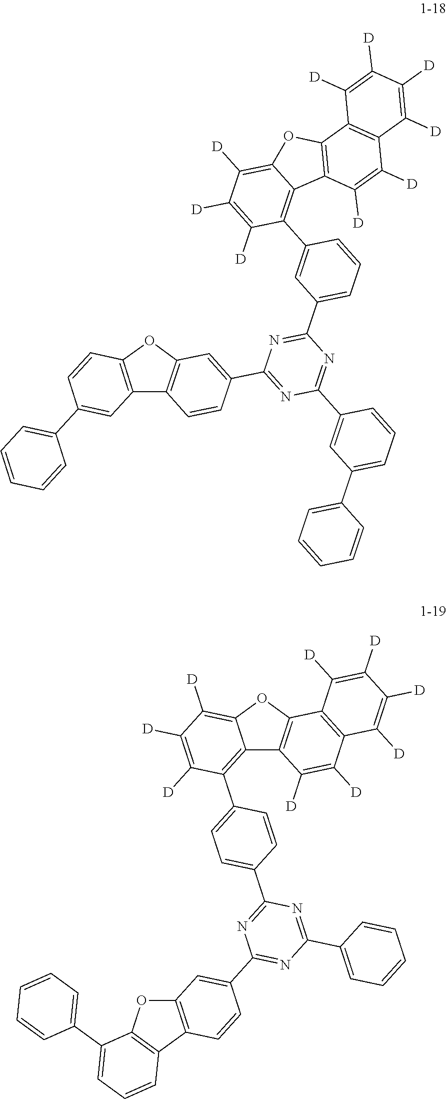 [SMILES: [2H]c1c([2H])c(-c2cccc(-c3nc(-c4cccc(-c5ccccc5)c4)nc(-c4ccc5c(c4)oc4ccc(-c6ccccc6)cc45)n3)c2)c2c(oc3c4c([2H])c([2H])c([2H])c([2H])c4c([2H])c([2H])c32)c1[2H] ; [2H]c1c([2H])c(-c2ccc(-c3nc(-c4ccccc4)nc(-c4ccc5c(c4)oc4c(-c6ccccc6)cccc45)n3)cc2)c2c(oc3c4c([2H])c([2H])c([2H])c([2H])c4c([2H])c([2H])c32)c1[2H]]

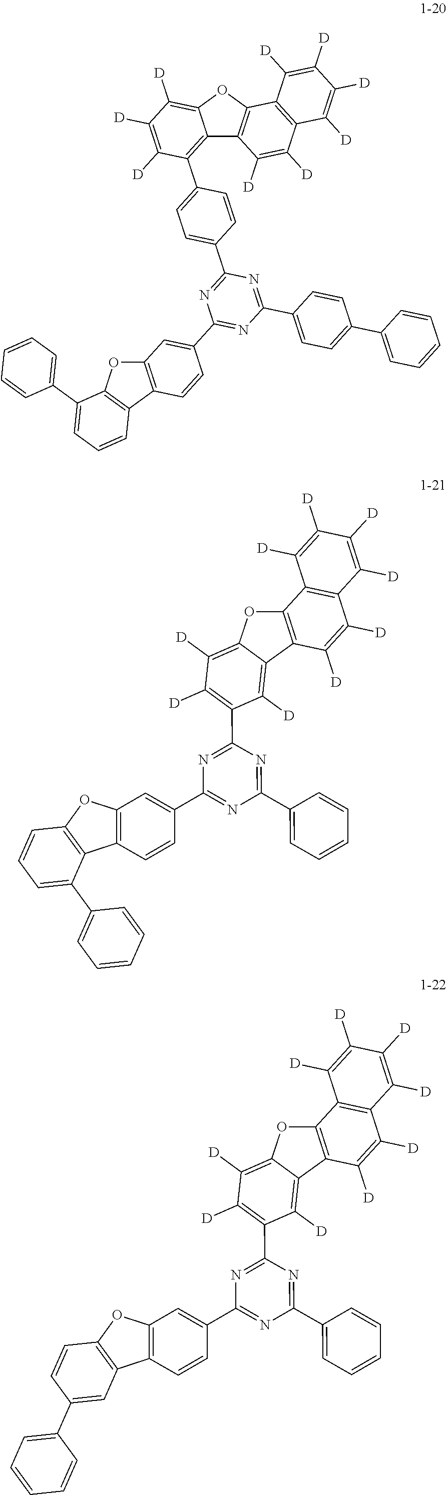 [SMILES: [2H]c1c([2H])c(-c2ccc(-c3nc(-c4ccc(-c5ccccc5)cc4)nc(-c4ccc5c(c4)oc4c(-c6ccccc6)cccc45)n3)cc2)c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)oc3ccc(-c5ccccc5)cc34)n2)c([2H])c2c(oc3c4c([2H])c([2H])c([2H])c([2H])c4c([2H])c([2H])c32)c1[2H]]

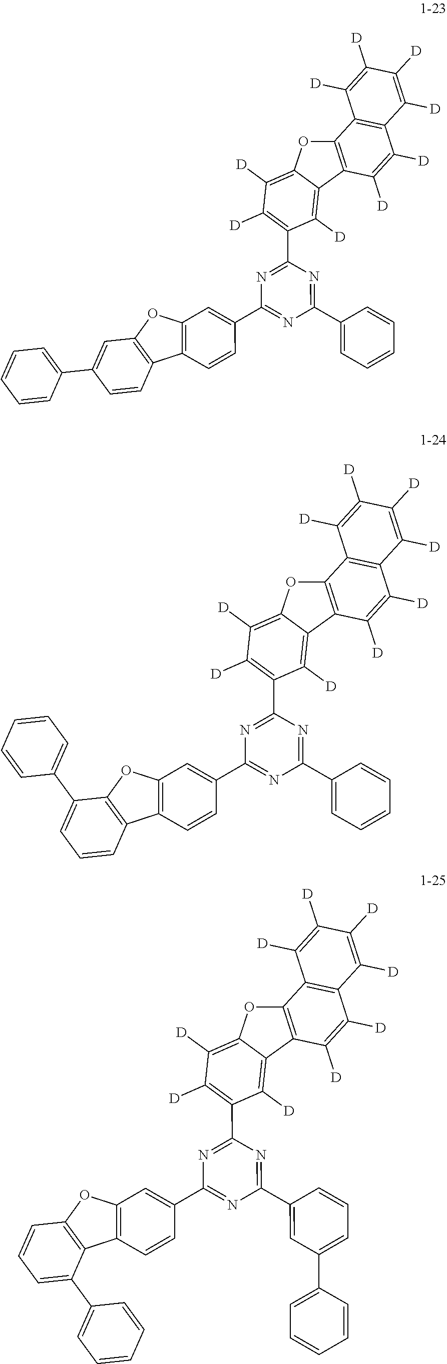 [SMILES: [2H]c1c(-c2nc(-c3ccccc3)nc(-c3ccc4c(c3)oc3cc(-c5ccccc5)ccc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)oc3c(-c5ccccc5)cccc34)n2)c([2H])c2c(oc3c4c([2H])c([2H])c([2H])c([2H])c4c([2H])c([2H])c32)c1[2H] ; [2H]c1c(-c2nc(-c3cccc(-c4ccccc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]]

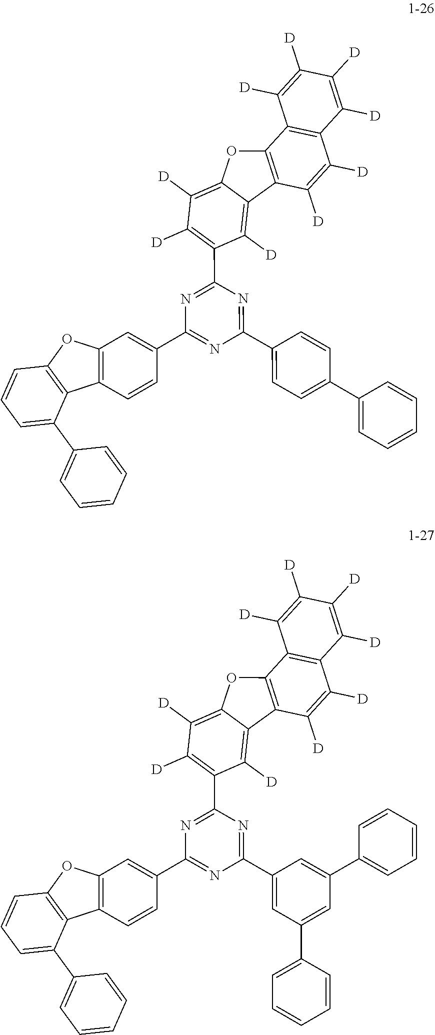 [SMILES: [2H]c1c(-c2nc(-c3ccc(-c4ccccc4)cc3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3cc(-c4ccccc4)cc(-c4ccccc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H]]

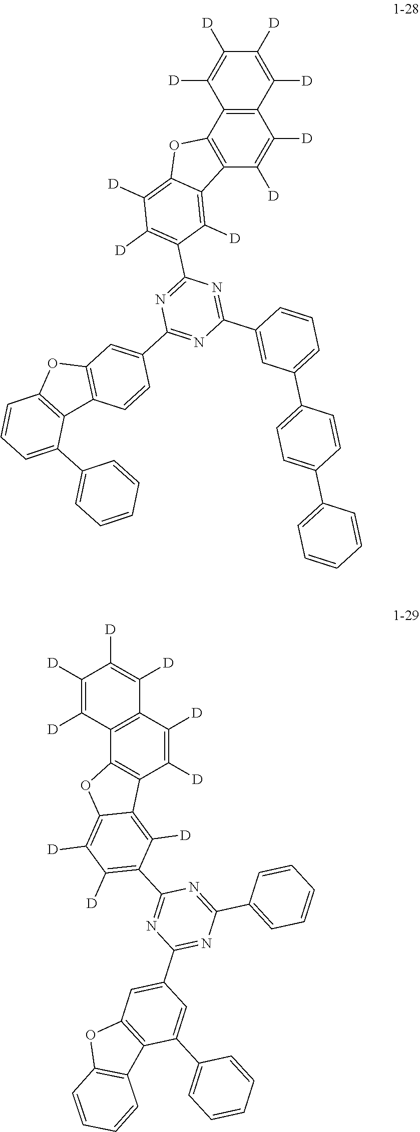 [SMILES: [2H]c1c(-c2nc(-c3cccc(-c4ccc(-c5ccccc5)cc4)c3)nc(-c3ccc4c(c3)oc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3cc(-c4ccccc4)c4c(c3)oc3ccccc34)n2)c([2H])c2c(oc3c4c([2H])c([2H])c([2H])c([2H])c4c([2H])c([2H])c32)c1[2H]]

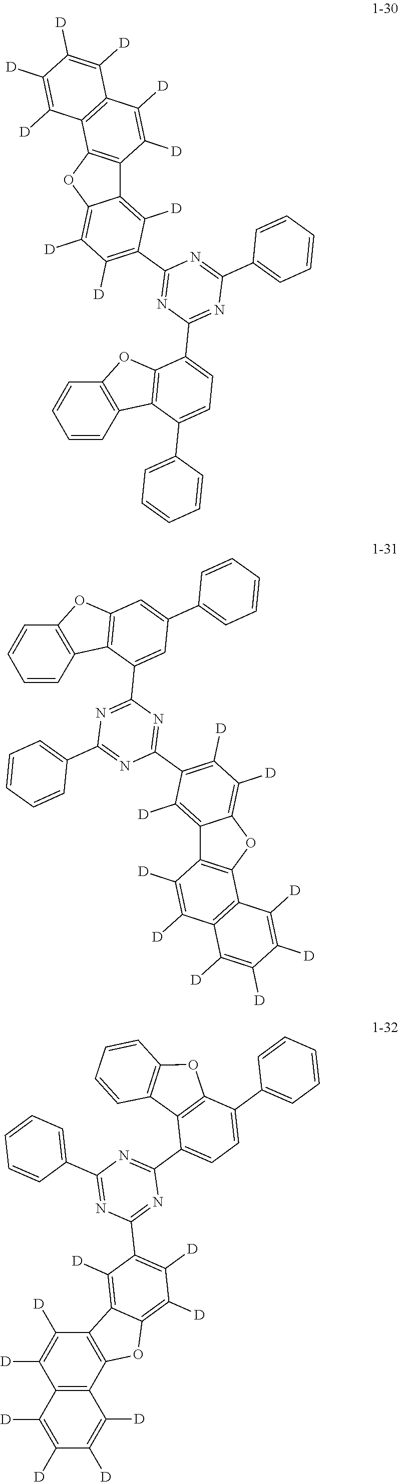

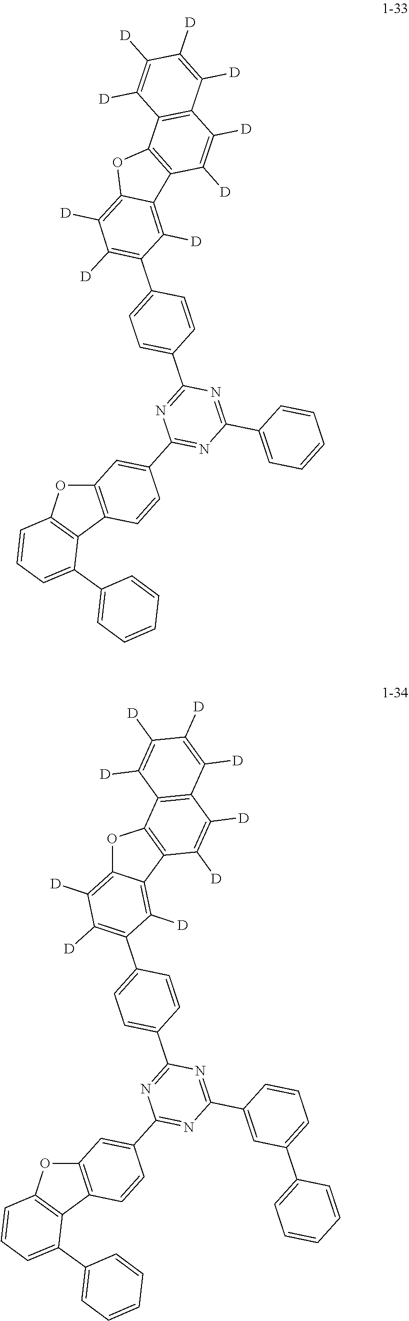 [SMILES: [2H]c1c(-c2ccc(-c3nc(-c4ccccc4)nc(-c4ccc5c(c4)oc4cccc(-c6ccccc6)c45)n3)cc2)c([2H])c2c(oc3c4c([2H])c([2H])c([2H])c([2H])c4c([2H])c([2H])c32)c1[2H] ; [2H]c1c(-c2ccc(-c3nc(-c4cccc(-c5ccccc5)c4)nc(-c4ccc5c(c4)oc4cccc(-c6ccccc6)c45)n3)cc2)c([2H])c2c(oc3c4c([2H])c([2H])c([2H])c([2H])c4c([2H])c([2H])c32)c1[2H]]

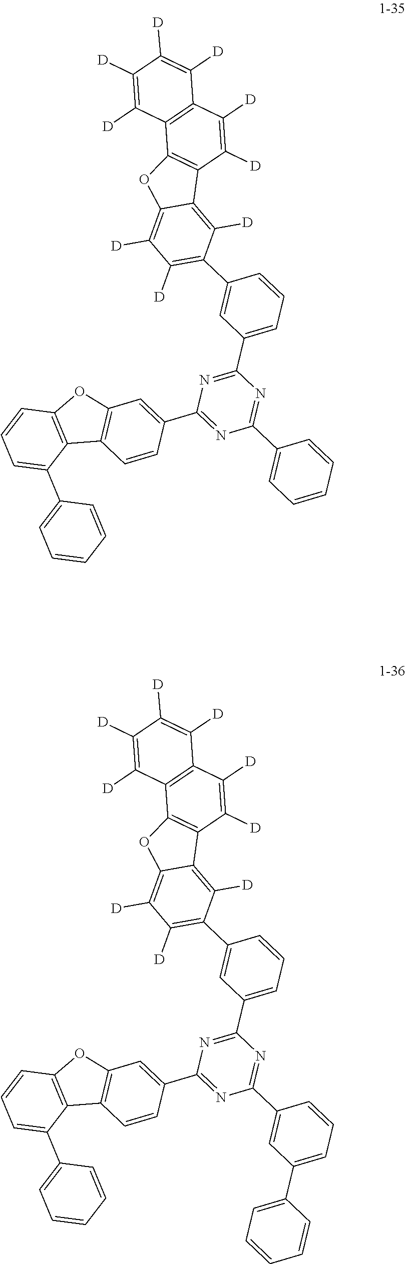 [SMILES: [2H]c1c(-c2cccc(-c3nc(-c4ccccc4)nc(-c4ccc5c(c4)oc4cccc(-c6ccccc6)c45)n3)c2)c([2H])c2c(oc3c4c([2H])c([2H])c([2H])c([2H])c4c([2H])c([2H])c32)c1[2H] ; [2H]c1c(-c2cccc(-c3nc(-c4cccc(-c5ccccc5)c4)nc(-c4ccc5c(c4)oc4cccc(-c6ccccc6)c45)n3)c2)c([2H])c2c(oc3c4c([2H])c([2H])c([2H])c([2H])c4c([2H])c([2H])c32)c1[2H]]

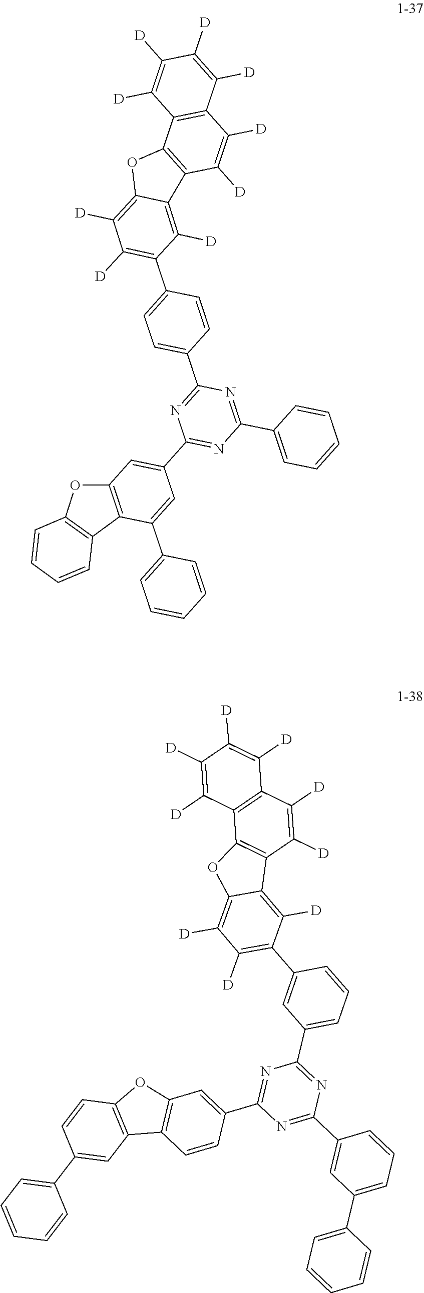 [SMILES: [2H]c1c(-c2ccc(-c3nc(-c4ccccc4)nc(-c4cc(-c5ccccc5)c5c(c4)oc4ccccc45)n3)cc2)c([2H])c2c(oc3c4c([2H])c([2H])c([2H])c([2H])c4c([2H])c([2H])c32)c1[2H] ; [2H]c1c(-c2cccc(-c3nc(-c4cccc(-c5ccccc5)c4)nc(-c4ccc5c(c4)oc4ccc(-c6ccccc6)cc45)n3)c2)c([2H])c2c(oc3c4c([2H])c([2H])c([2H])c([2H])c4c([2H])c([2H])c32)c1[2H]]

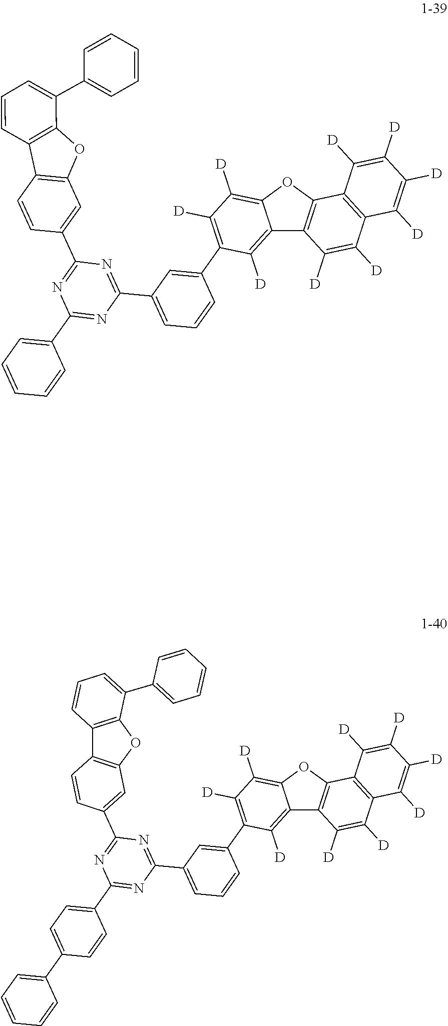 [SMILES: [2H]c1c(-c2cccc(-c3nc(-c4ccccc4)nc(-c4ccc5c(c4)oc4c(-c6ccccc6)cccc45)n3)c2)c([2H])c2c(oc3c4c([2H])c([2H])c([2H])c([2H])c4c([2H])c([2H])c32)c1[2H] ; [2H]c1c(-c2cccc(-c3nc(-c4ccc(-c5ccccc5)cc4)nc(-c4ccc5c(c4)oc4c(-c6ccccc6)cccc45)n3)c2)c([2H])c2c(oc3c4c([2H])c([2H])c([2H])c([2H])c4c([2H])c([2H])c32)c1[2H]]

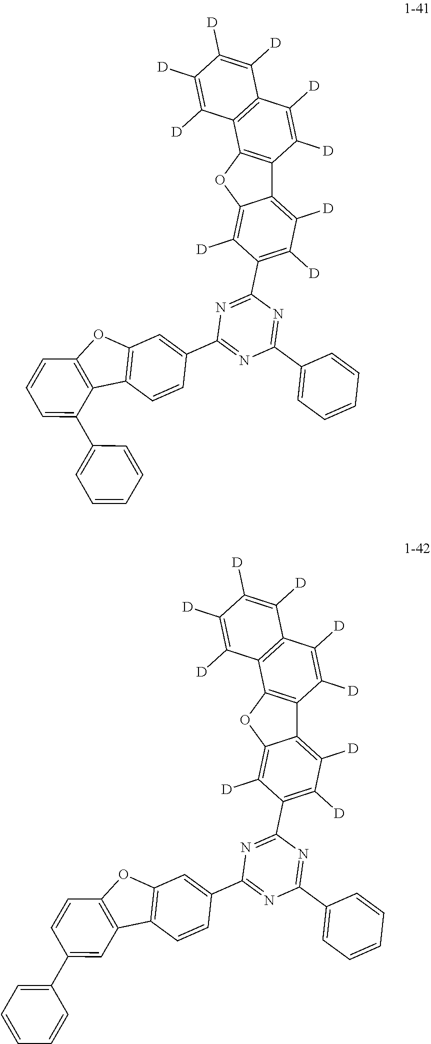 [SMILES: [2H]c1c([2H])c([2H])c2c(c1[2H])c([2H])c([2H])c1c2oc2c([2H])c(-c3nc(-c4ccccc4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)oc4ccc(-c6ccccc6)cc45)n3)c([2H])c([2H])c21]

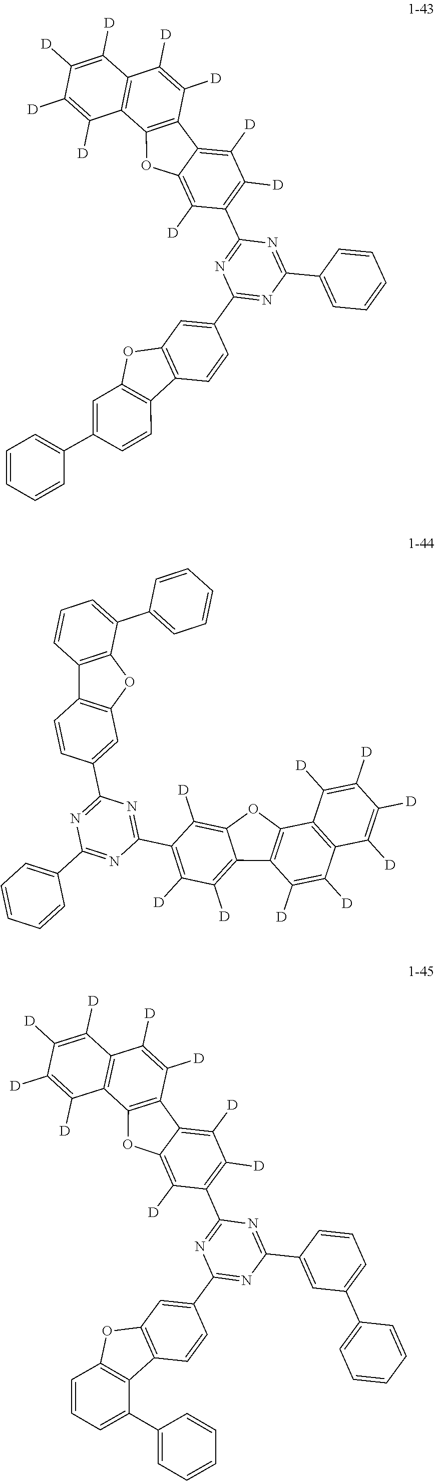 [SMILES: [2H]c1c([2H])c([2H])c2c(c1[2H])c([2H])c([2H])c1c2oc2c([2H])c(-c3nc(-c4ccccc4)nc(-c4ccc5c(c4)oc4cc(-c6ccccc6)ccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)oc4c(-c6ccccc6)cccc45)n3)c([2H])c([2H])c21 ; [2H]c1c([2H])c([2H])c2c(c1[2H])c([2H])c([2H])c1c2oc2c([2H])c(-c3nc(-c4cccc(-c5ccccc5)c4)nc(-c4ccc5c(c4)oc4cccc(-c6ccccc6)c45)n3)c([2H])c([2H])c21]

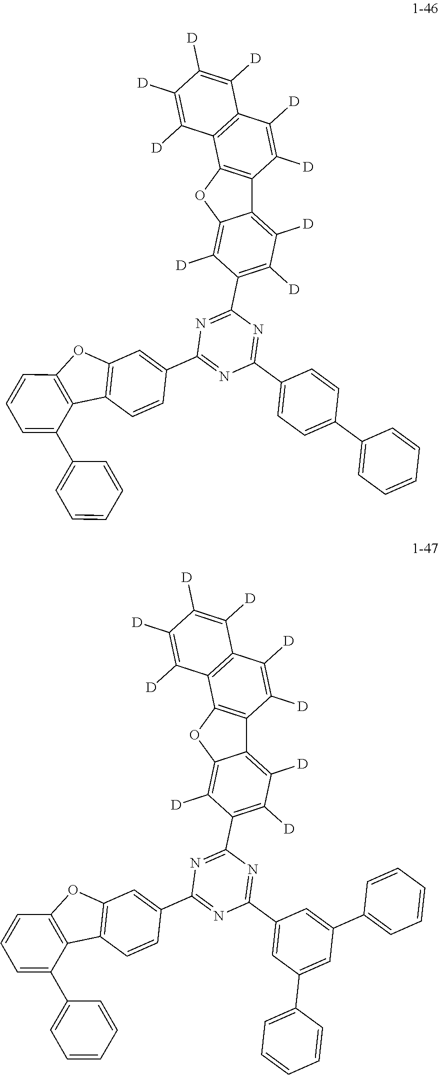 [SMILES: [2H]c1c([2H])c([2H])c2c(c1[2H])c([2H])c([2H])c1c2oc2c([2H])c(-c3nc(-c4ccc(-c5ccccc5)cc4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4cc(-c5ccccc5)cc(-c5ccccc5)c4)nc(-c4ccc5c(c4)oc4cccc(-c6ccccc6)c45)n3)c([2H])c([2H])c21]

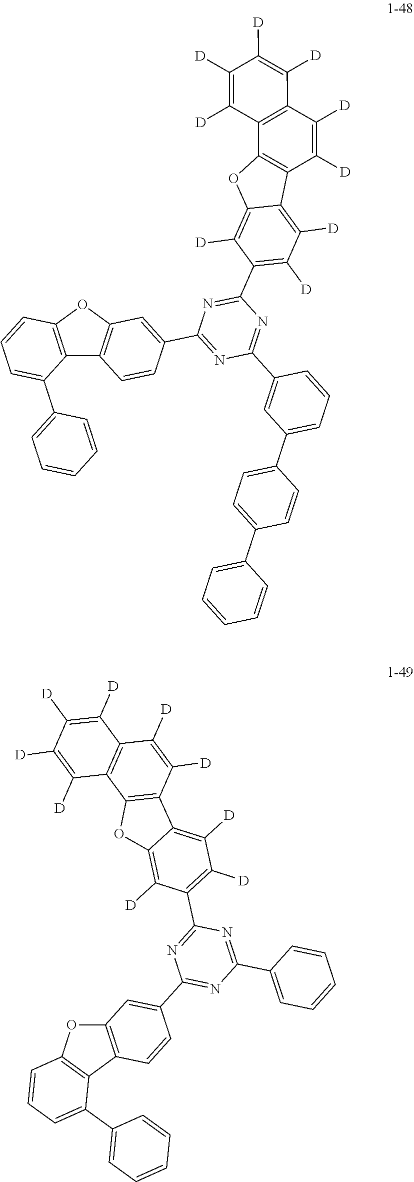 [SMILES: [2H]c1c([2H])c([2H])c2c(c1[2H])c([2H])c([2H])c1c2oc2c([2H])c(-c3nc(-c4cccc(-c5ccc(-c6ccccc6)cc5)c4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)oc4cccc(-c6ccccc6)c45)n3)c([2H])c([2H])c21]

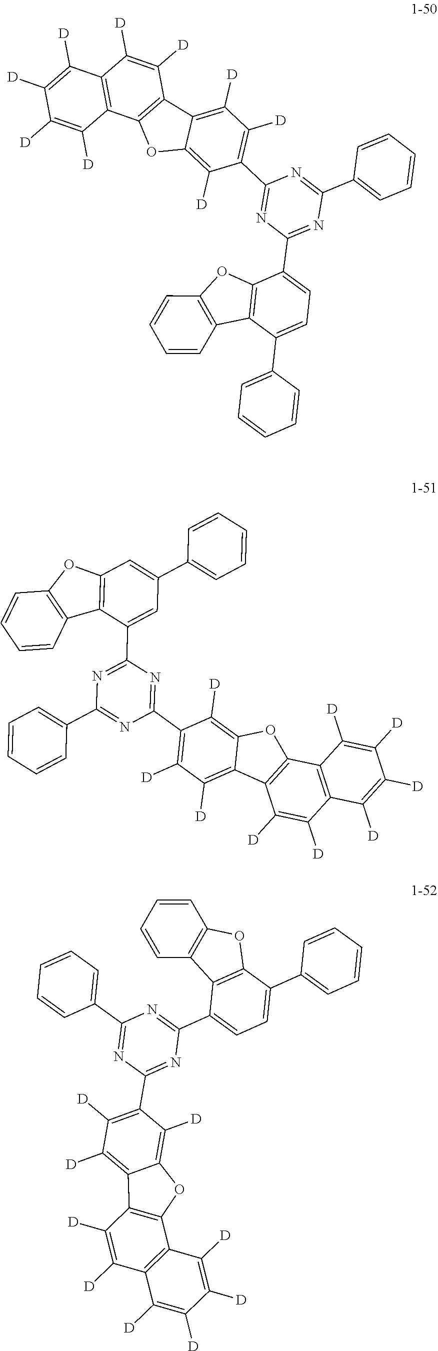 [SMILES: [2H]c1c([2H])c([2H])c2c(c1[2H])c([2H])c([2H])c1c2oc2c([2H])c(-c3nc(-c4ccccc4)nc(-c4ccc(-c5ccccc5)c5c4oc4ccccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4cc(-c5ccccc5)cc5oc6ccccc6c45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc(-c5ccccc5)c5oc6ccccc6c45)n3)c([2H])c([2H])c21]

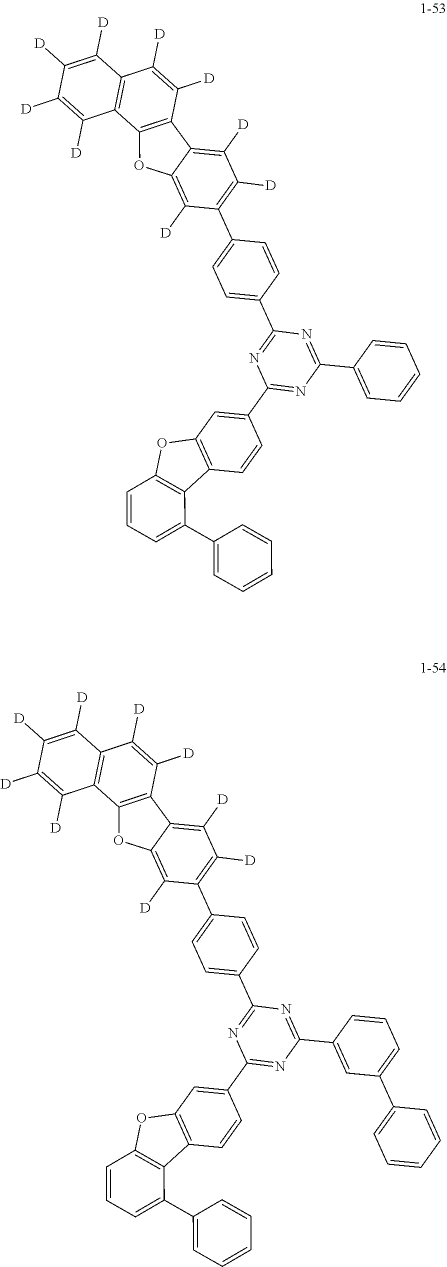 [SMILES: [2H]c1c([2H])c([2H])c2c(c1[2H])c([2H])c([2H])c1c2oc2c([2H])c(-c3ccc(-c4nc(-c5ccccc5)nc(-c5ccc6c(c5)oc5cccc(-c7ccccc7)c56)n4)cc3)c([2H])c([2H])c21 ; [2H]c1c([2H])c([2H])c2c(c1[2H])c([2H])c([2H])c1c2oc2c([2H])c(-c3ccc(-c4nc(-c5cccc(-c6ccccc6)c5)nc(-c5ccc6c(c5)oc5cccc(-c7ccccc7)c56)n4)cc3)c([2H])c([2H])c21]

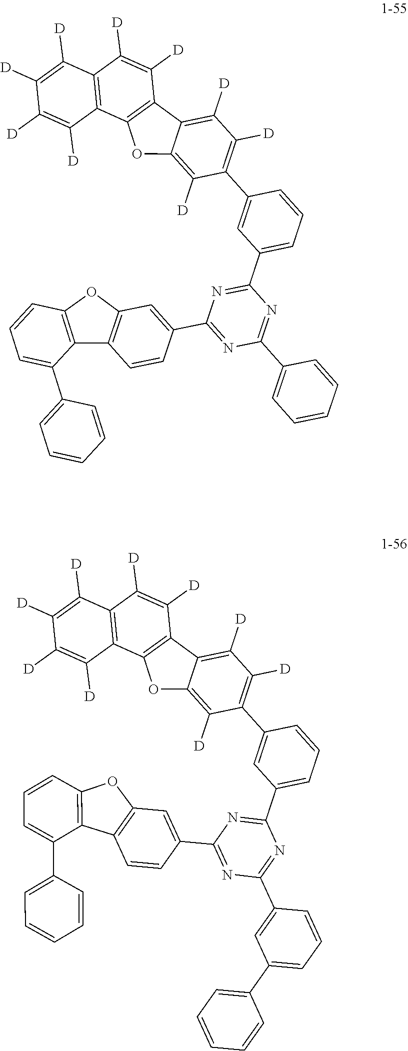 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)oc5cccc(-c7ccccc7)c56)n4)c3)c([2H])c([2H])c21 ; [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)oc5cccc(-c7ccccc7)c56)n4)c3)c([2H])c([2H])c21]

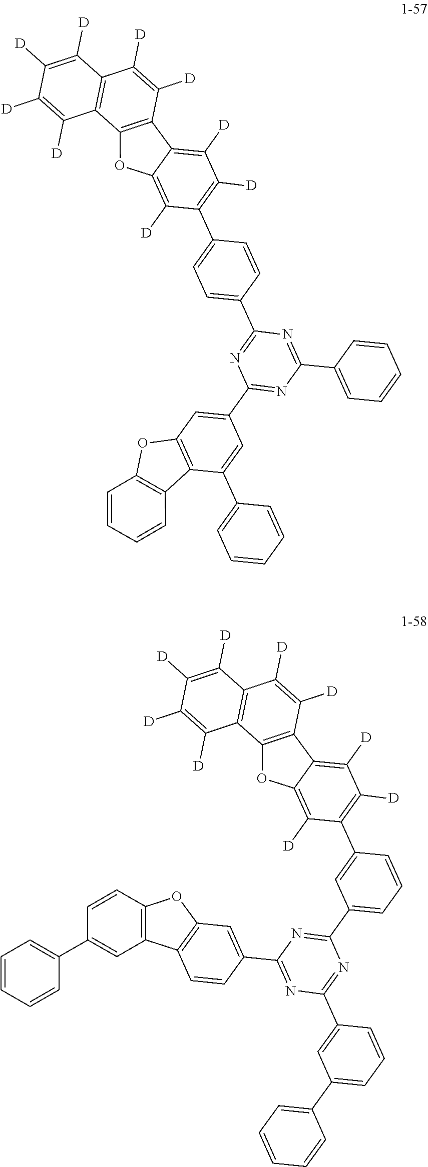 [SMILES: [2H]c1c([2H])c([2H])c2c(c1[2H])c([2H])c([2H])c1c2oc2c([2H])c(-c3ccc(-c4nc(-c5ccccc5)nc(-c5cc(-c6ccccc6)c6c(c5)oc5ccccc56)n4)cc3)c([2H])c([2H])c21 ; [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)oc5ccc(-c7ccccc7)cc56)n4)c3)c([2H])c([2H])c21]

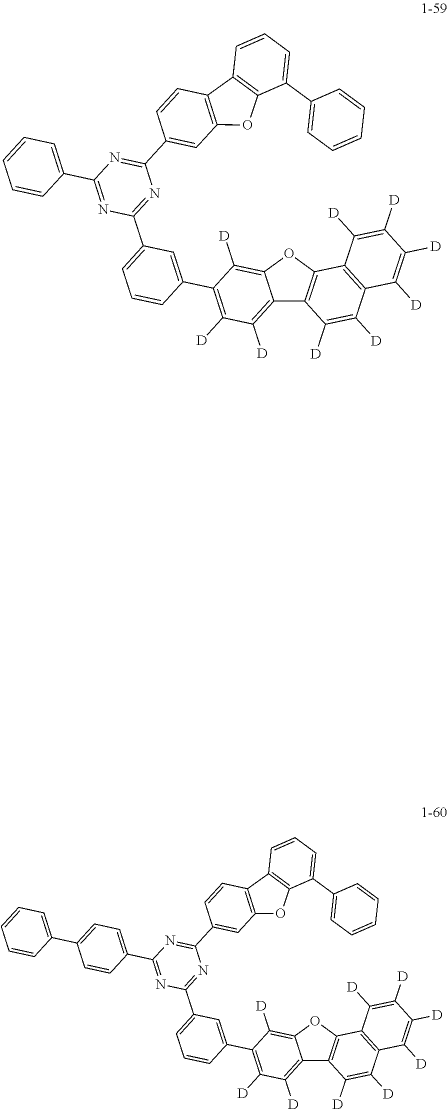 [SMILES: [2H]c1c([2H])c([2H])c2c(c1[2H])c([2H])c([2H])c1c2oc2c([2H])c(-c3cccc(-c4nc(-c5ccccc5)nc(-c5ccc6c(c5)oc5c(-c7ccccc7)cccc56)n4)c3)c([2H])c([2H])c21 ; [2H]c1c([2H])c([2H])c2c(c1[2H])c([2H])c([2H])c1c2oc2c([2H])c(-c3cccc(-c4nc(-c5ccc(-c6ccccc6)cc5)nc(-c5ccc6c(c5)oc5c(-c7ccccc7)cccc56)n4)c3)c([2H])c([2H])c21]

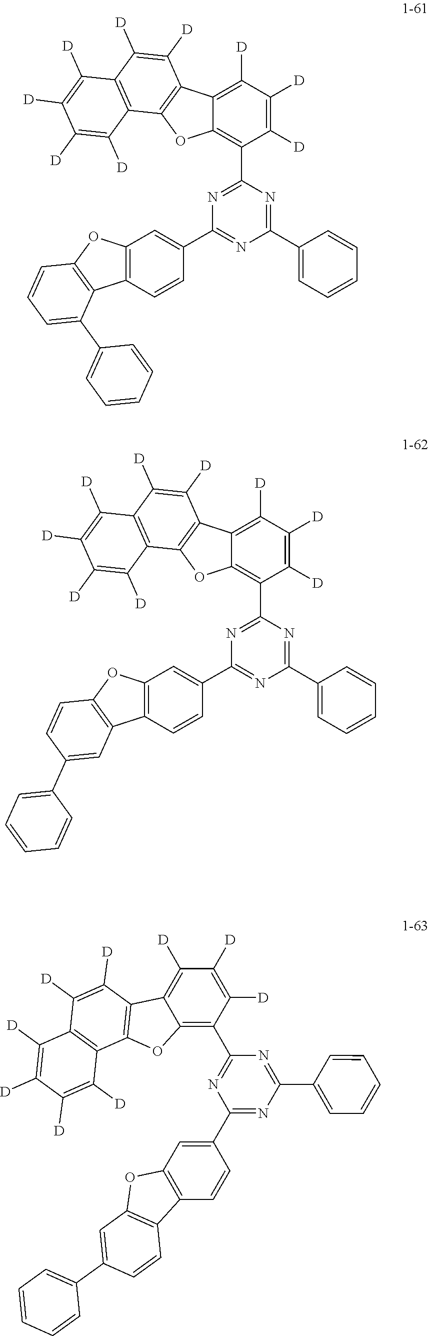 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2ccc(-c4ccccc4)cc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2cc(-c4ccccc4)ccc23)n1]

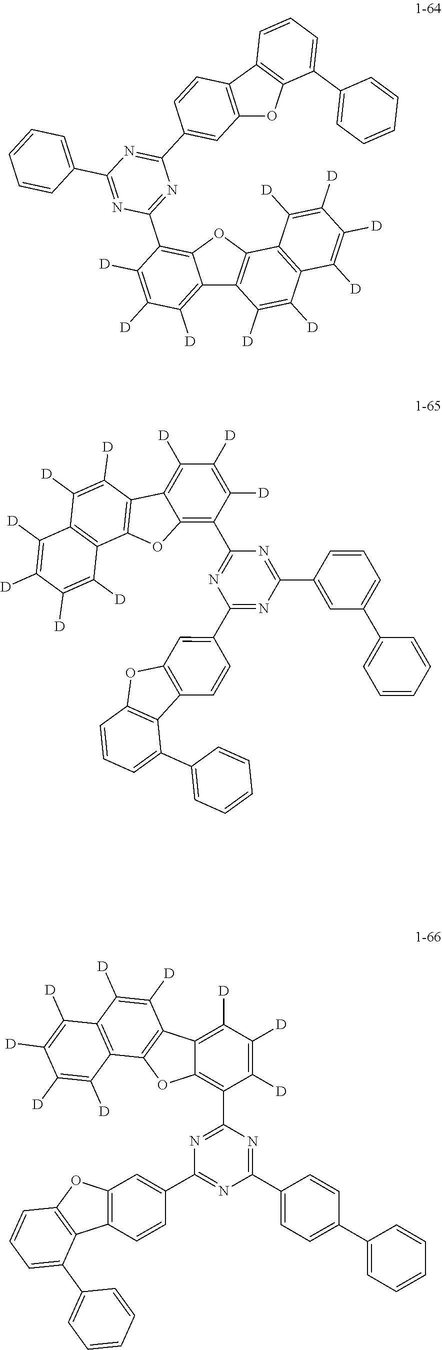 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)oc2c(-c4ccccc4)cccc23)n1 ; [2H]c1c([2H])c([2H])c2c(oc3c4c([2H])c([2H])c([2H])c([2H])c4c([2H])c([2H])c32)c1-c1nc(-c2cccc(-c3ccccc3)c2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccc(-c3ccccc3)cc2)nc(-c2ccc3c(c2)oc2cccc(-c4ccccc4)c23)n1]

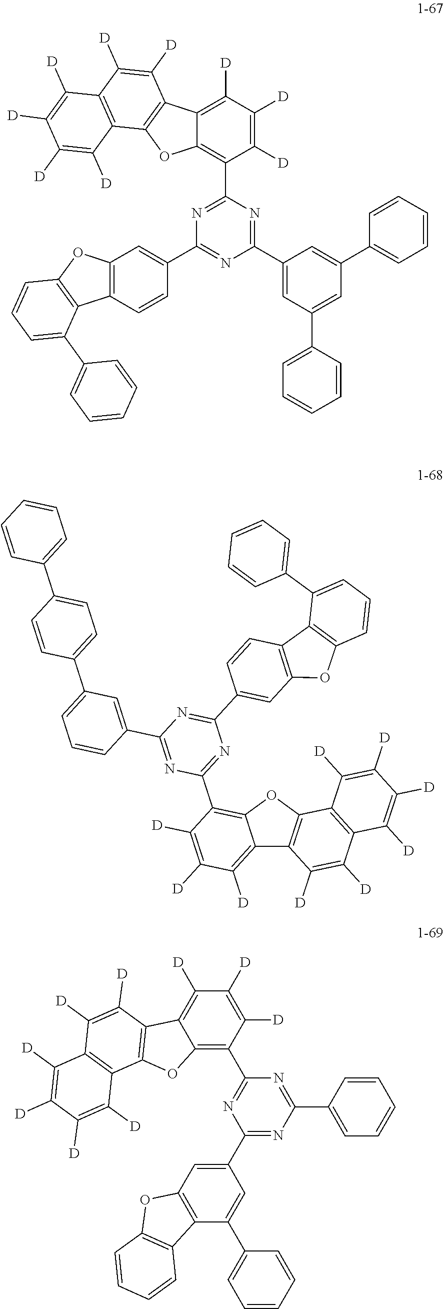 [SMILES: [2H]c1c([2H])c([2H])c2c(oc3c4c([2H])c([2H])c([2H])c([2H])c4c([2H])c([2H])c32)c1-c1nc(-c2cc(-c3ccccc3)cc(-c3ccccc3)c2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2cccc(-c3ccc(-c4ccccc4)cc3)c2)nc(-c2ccc3c(c2)oc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)c3c(c2)oc2ccccc23)n1]

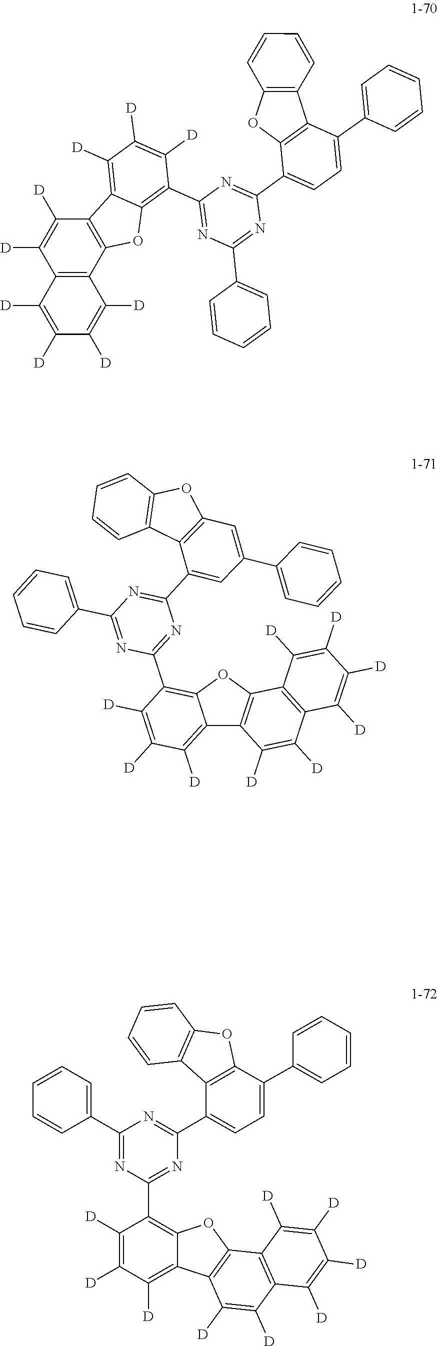 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3c2oc2ccccc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)cc3oc4ccccc4c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3oc4ccccc4c23)n1]

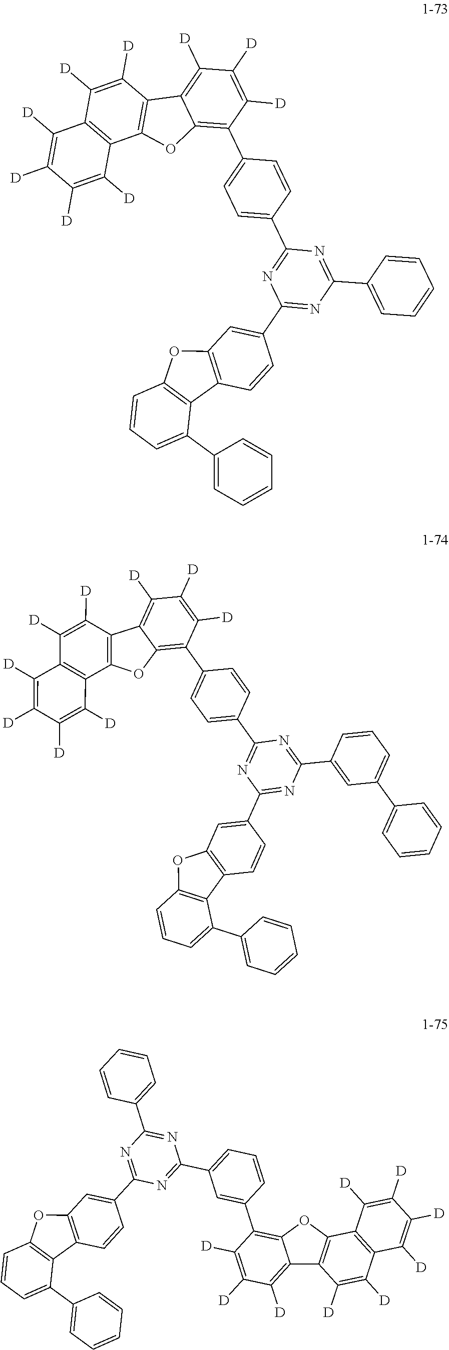 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3ccc4c(c3)oc3cccc(-c5ccccc5)c34)n2)cc1 ; [2H]c1c([2H])c([2H])c2c(oc3c4c([2H])c([2H])c([2H])c([2H])c4c([2H])c([2H])c32)c1-c1ccc(-c2nc(-c3cccc(-c4ccccc4)c3)nc(-c3ccc4c(c3)oc3cccc(-c5ccccc5)c34)n2)cc1 ; [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)oc3cccc(-c5ccccc5)c34)n2)c1]

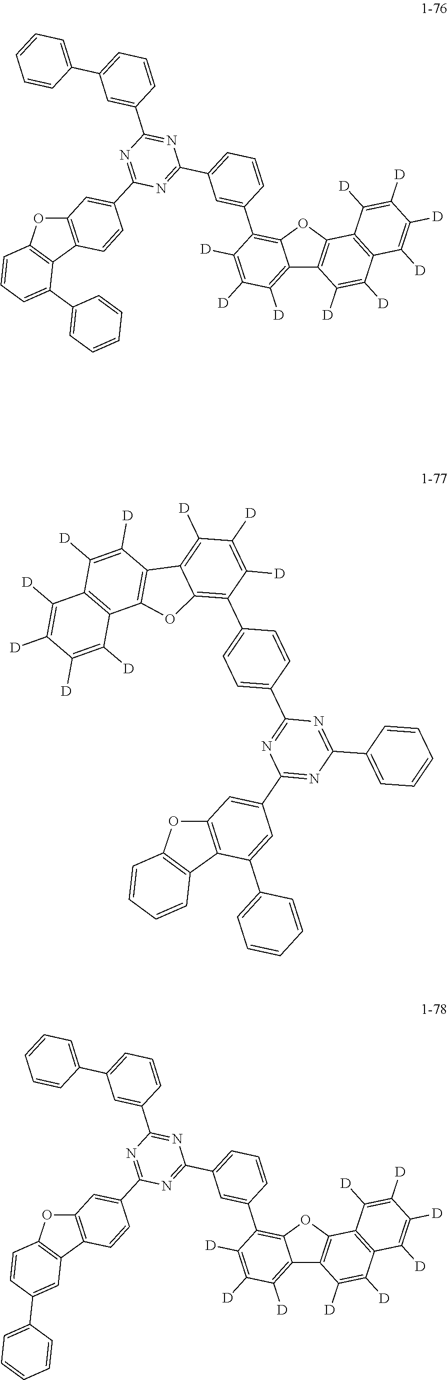 [SMILES: [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3cccc(-c5ccccc5)c34)n2)c1 ; [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3cc(-c4ccccc4)c4c(c3)oc3ccccc34)n2)cc1 ; [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3ccc(-c5ccccc5)cc34)n2)c1]

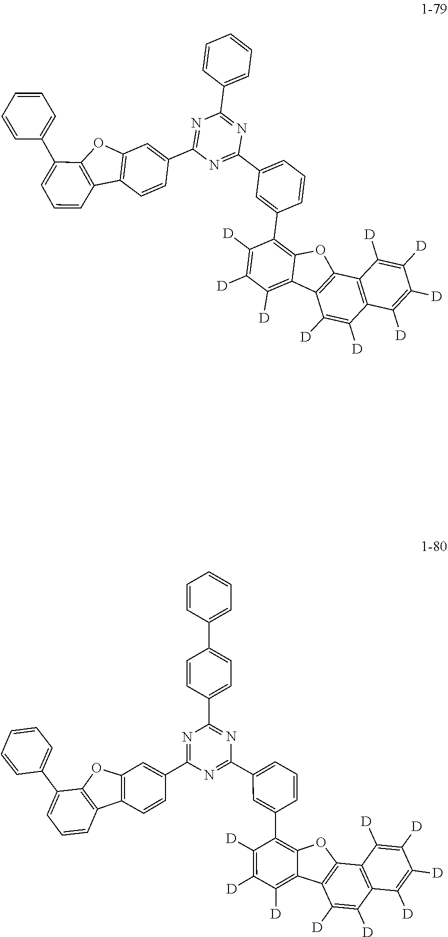 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)oc3c(-c5ccccc5)cccc34)n2)c1 ; [2H]c1c([2H])c([2H])c2c(oc3c4c([2H])c([2H])c([2H])c([2H])c4c([2H])c([2H])c32)c1-c1cccc(-c2nc(-c3ccc(-c4ccccc4)cc3)nc(-c3ccc4c(c3)oc3c(-c5ccccc5)cccc34)n2)c1]

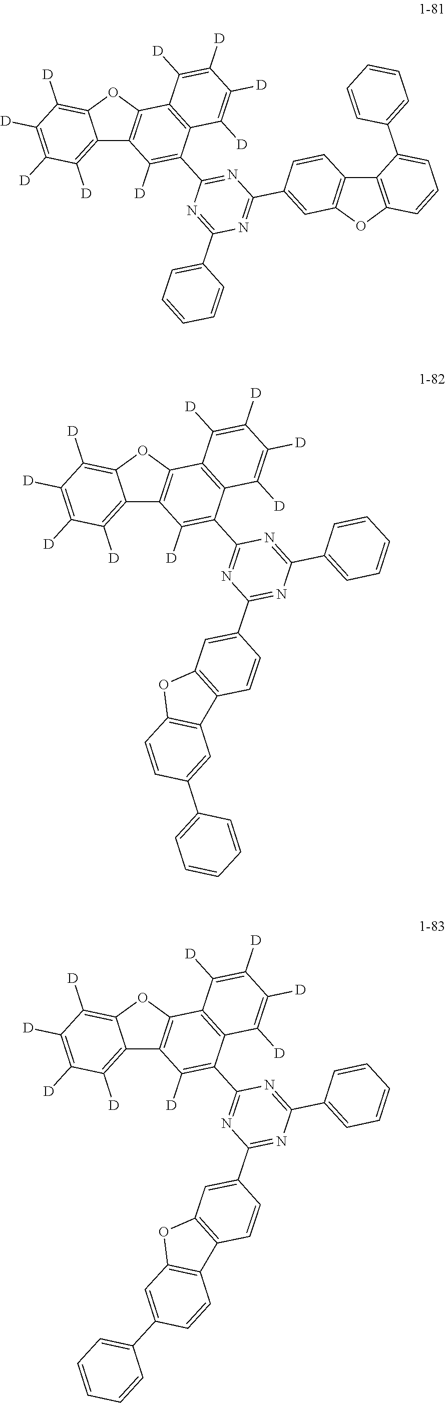

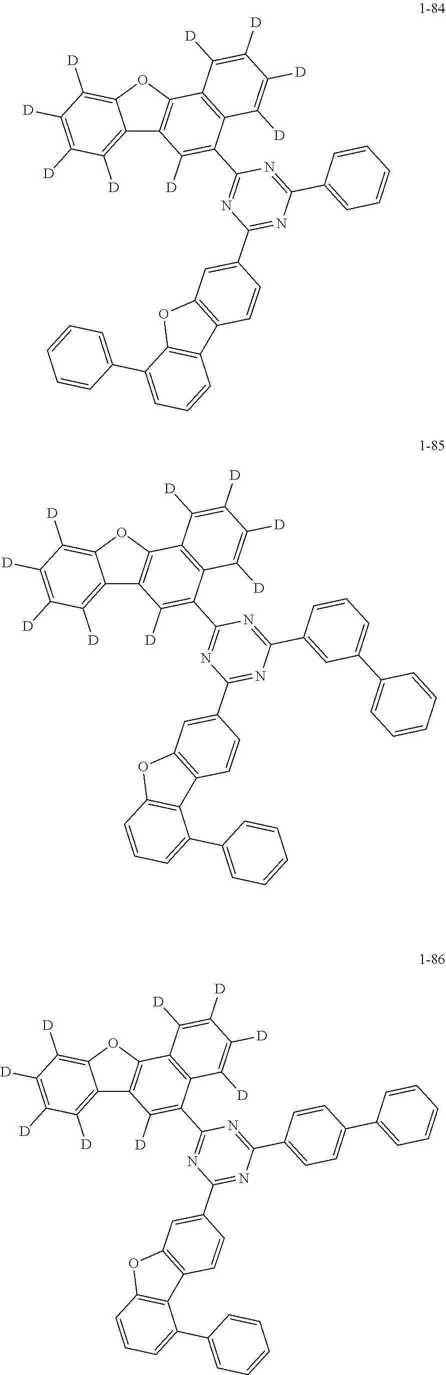

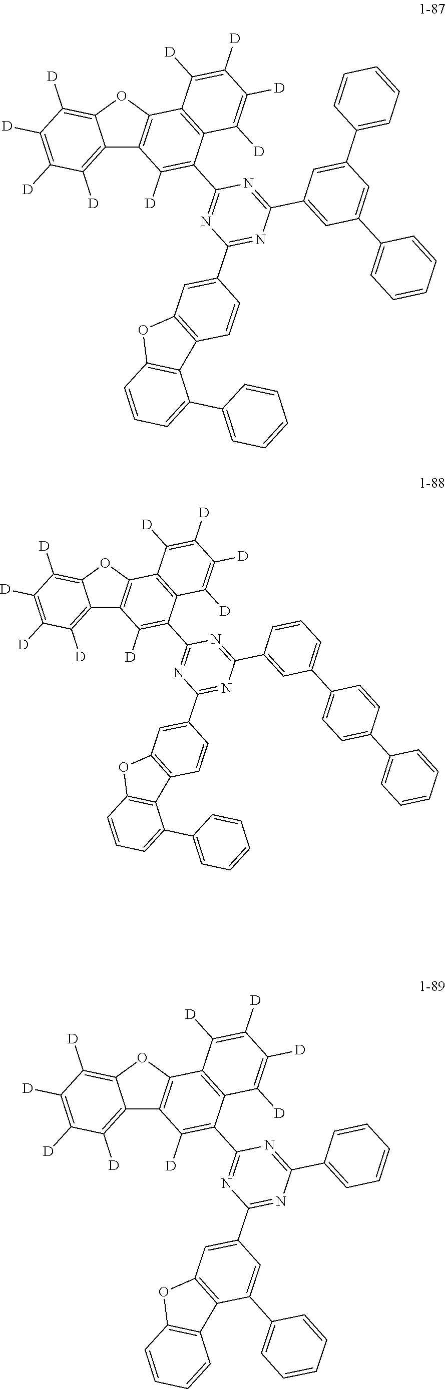

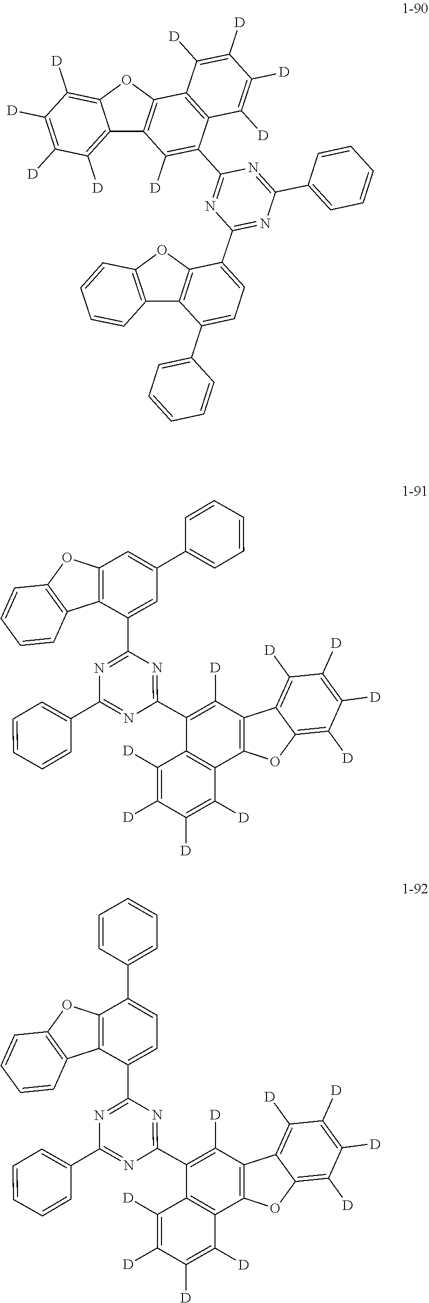

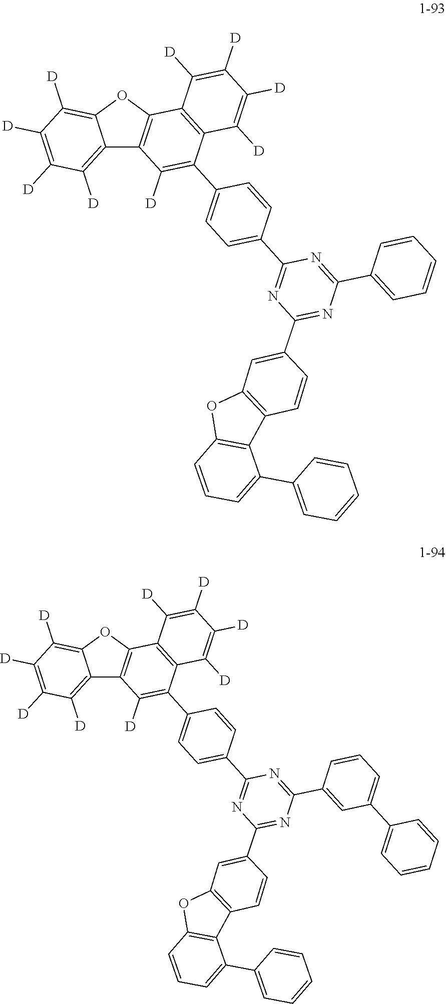 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]]

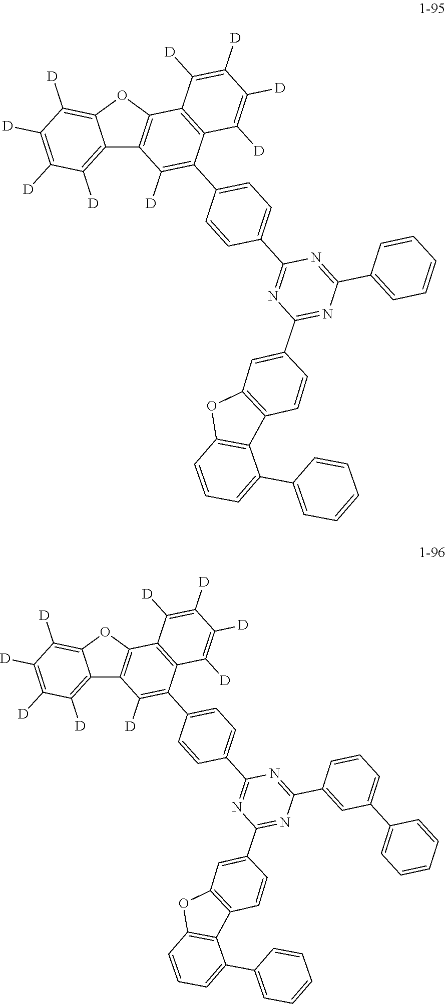 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H]]

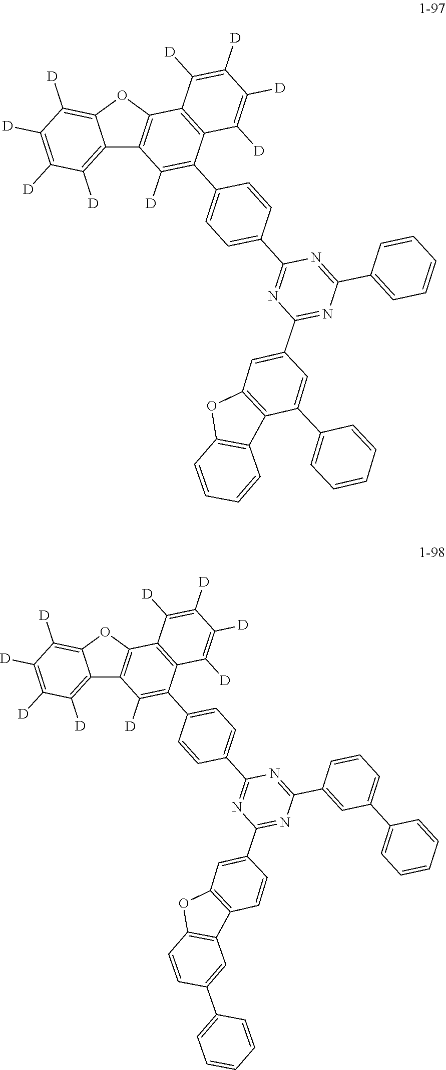 [SMILES: [2H]c1c([2H])c([2H])c2c(oc3c4c([2H])c([2H])c([2H])c([2H])c4c(-c4ccc(-c5nc(-c6ccccc6)nc(-c6cc(-c7ccccc7)c7c(c6)oc6ccccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)oc6ccc(-c8ccccc8)cc67)n5)cc4)c([2H])c32)c1[2H]]

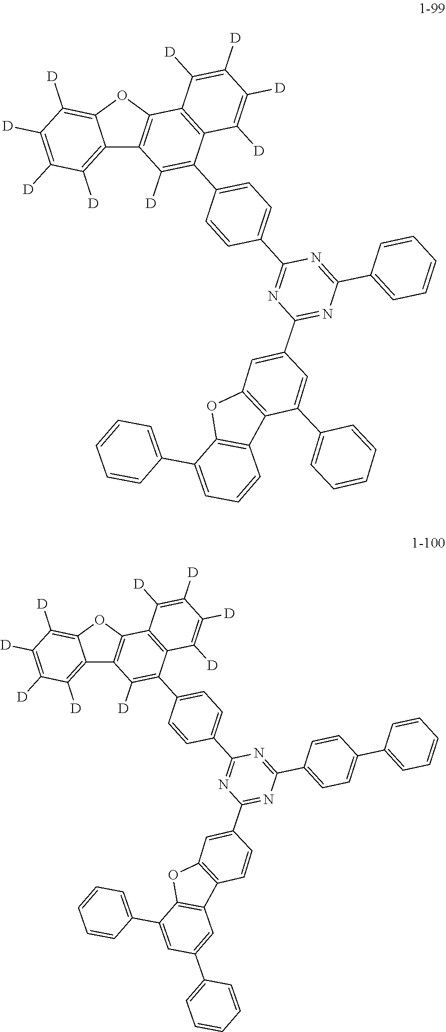 [SMILES: [2H]c1c([2H])c([2H])c2c(oc3c4c([2H])c([2H])c([2H])c([2H])c4c(-c4ccc(-c5nc(-c6ccccc6)nc(-c6cc(-c7ccccc7)c7c(c6)oc6c(-c8ccccc8)cccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6ccc(-c7ccccc7)cc6)nc(-c6ccc7c(c6)oc6c(-c8ccccc8)cc(-c8ccccc8)cc67)n5)cc4)c([2H])c32)c1[2H]]

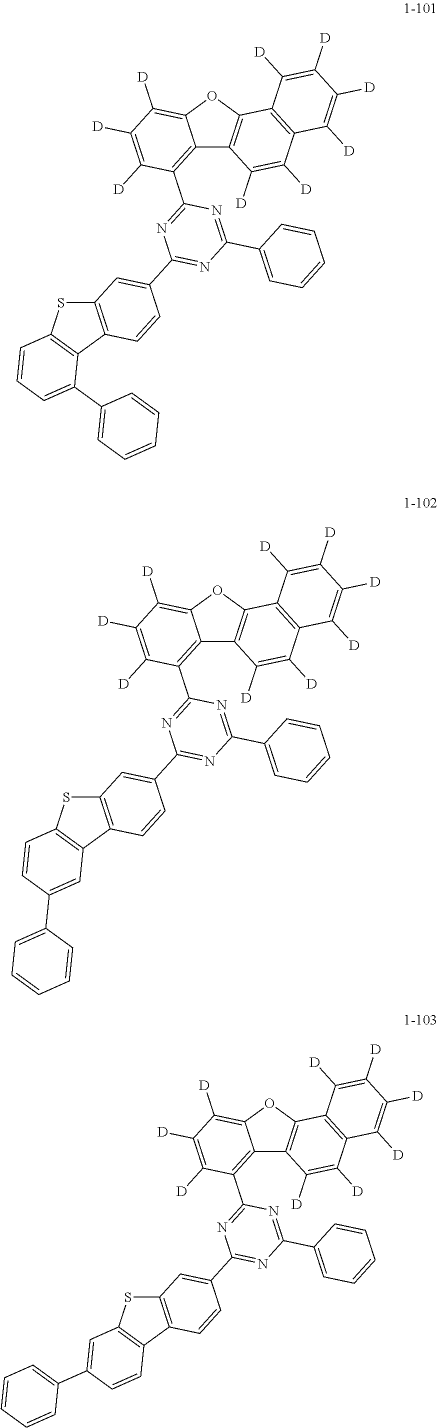 [SMILES: [2H]c1c([2H])c(-c2nc(-c3ccccc3)nc(-c3ccc4c(c3)sc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)sc3ccc(-c5ccccc5)cc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)sc3cc(-c5ccccc5)ccc34)n2)c2c(oc3c4c([2H])c([2H])c([2H])c([2H])c4c([2H])c([2H])c32)c1[2H]]

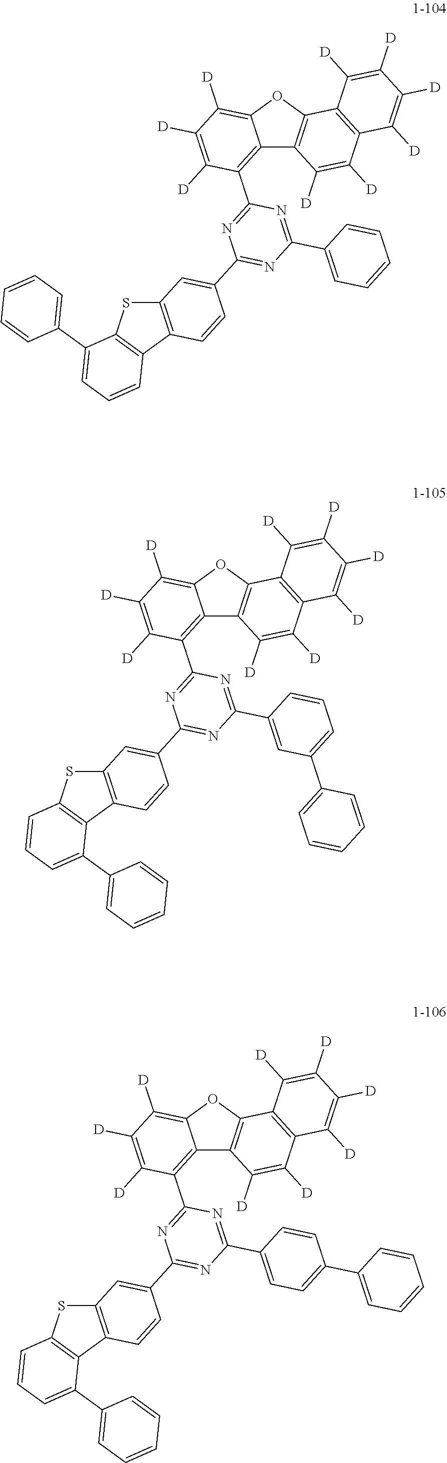 [SMILES: [2H]c1c([2H])c(-c2nc(-c3ccccc3)nc(-c3ccc4c(c3)sc3c(-c5ccccc5)cccc34)n2)c2c(oc3c4c([2H])c([2H])c([2H])c([2H])c4c([2H])c([2H])c32)c1[2H] ; [2H]c1c([2H])c(-c2nc(-c3cccc(-c4ccccc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccc(-c4ccccc4)cc3)nc(-c3ccc4c(c3)sc3cccc(-c5ccccc5)c34)n2)c2c(oc3c4c([2H])c([2H])c([2H])c([2H])c4c([2H])c([2H])c32)c1[2H]]

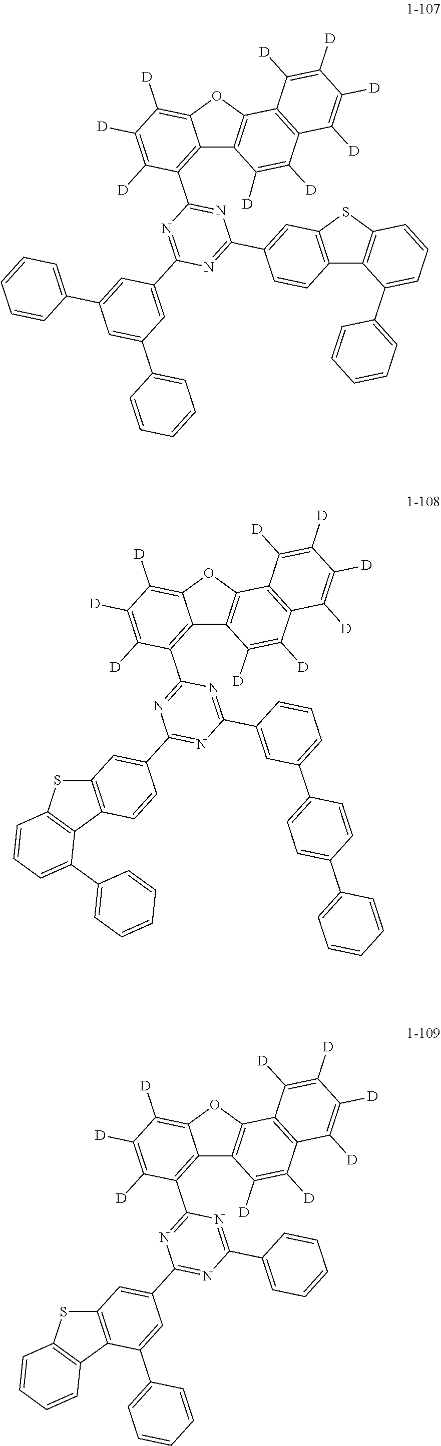 [SMILES: [2H]c1c([2H])c(-c2nc(-c3cc(-c4ccccc4)cc(-c4ccccc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3cccc(-c4ccc(-c5ccccc5)cc4)c3)nc(-c3ccc4c(c3)sc3cccc(-c5ccccc5)c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3cc(-c4ccccc4)c4c(c3)sc3ccccc34)n2)c2c(oc3c4c([2H])c([2H])c([2H])c([2H])c4c([2H])c([2H])c32)c1[2H]]

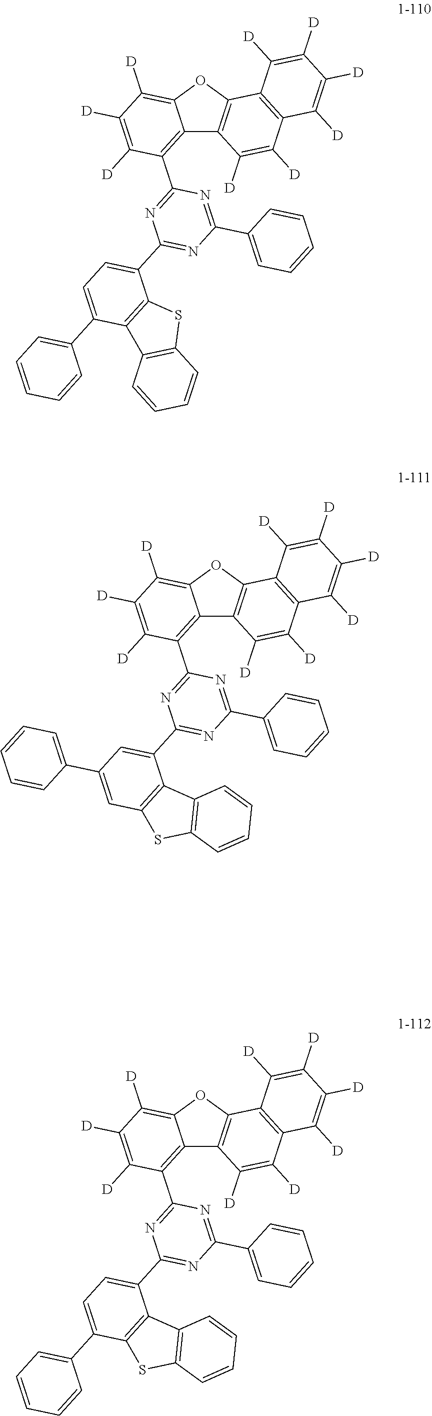 [SMILES: [2H]c1c([2H])c(-c2nc(-c3ccccc3)nc(-c3ccc(-c4ccccc4)c4c3sc3ccccc34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3cc(-c4ccccc4)cc4sc5ccccc5c34)n2)c2c(oc3c4c([2H])c([2H])c([2H])c([2H])c4c([2H])c([2H])c32)c1[2H] ; [2H]c1c([2H])c(-c2nc(-c3ccccc3)nc(-c3ccc(-c4ccccc4)c4sc5ccccc5c34)n2)c2c(oc3c4c([2H])c([2H])c([2H])c([2H])c4c([2H])c([2H])c32)c1[2H]]

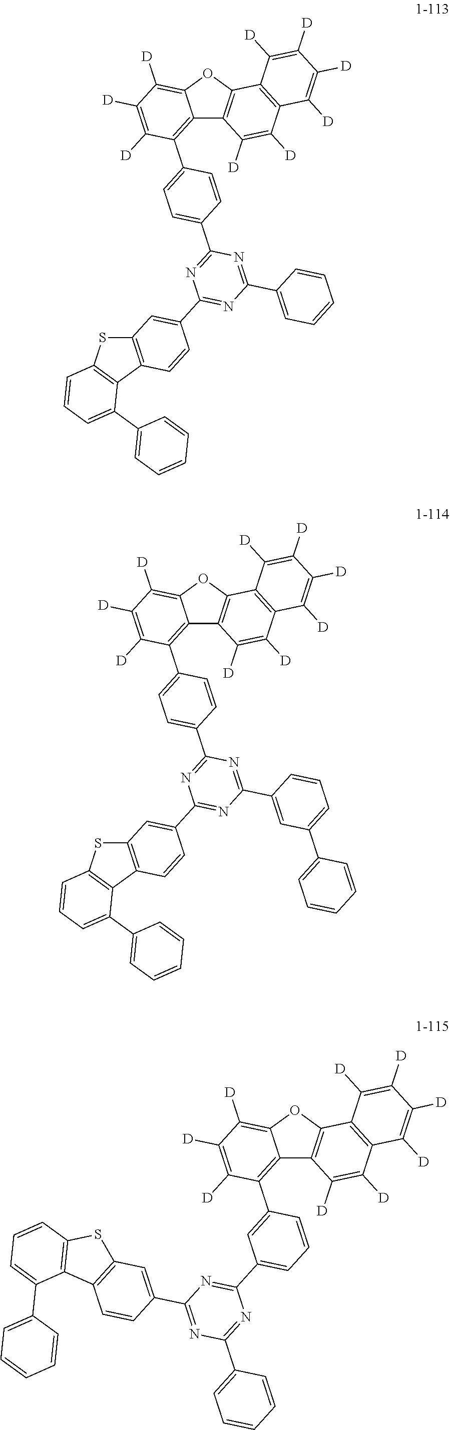 [SMILES: [2H]c1c([2H])c(-c2ccc(-c3nc(-c4ccccc4)nc(-c4ccc5c(c4)sc4cccc(-c6ccccc6)c45)n3)cc2)c2c(oc3c4c([2H])c([2H])c([2H])c([2H])c4c([2H])c([2H])c32)c1[2H] ; [2H]c1c([2H])c(-c2ccc(-c3nc(-c4cccc(-c5ccccc5)c4)nc(-c4ccc5c(c4)sc4cccc(-c6ccccc6)c45)n3)cc2)c2c(oc3c4c([2H])c([2H])c([2H])c([2H])c4c([2H])c([2H])c32)c1[2H] ; [2H]c1c([2H])c(-c2cccc(-c3nc(-c4ccccc4)nc(-c4ccc5c(c4)sc4cccc(-c6ccccc6)c45)n3)c2)c2c(oc3c4c([2H])c([2H])c([2H])c([2H])c4c([2H])c([2H])c32)c1[2H]]

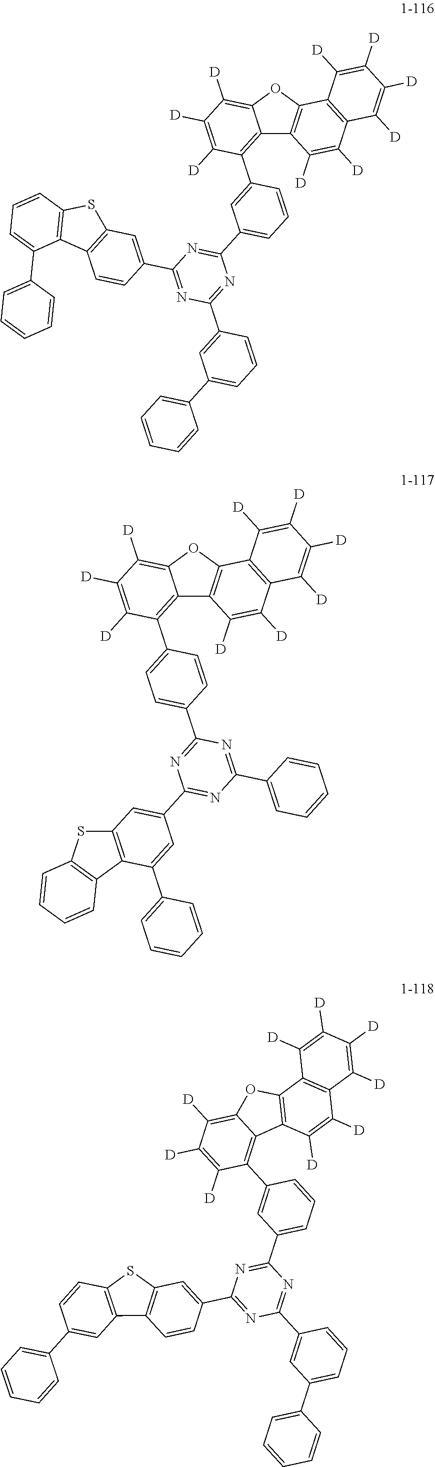 [SMILES: [2H]c1c([2H])c(-c2cccc(-c3nc(-c4cccc(-c5ccccc5)c4)nc(-c4ccc5c(c4)sc4cccc(-c6ccccc6)c45)n3)c2)c2c(oc3c4c([2H])c([2H])c([2H])c([2H])c4c([2H])c([2H])c32)c1[2H] ; [2H]c1c([2H])c(-c2ccc(-c3nc(-c4ccccc4)nc(-c4cc(-c5ccccc5)c5c(c4)sc4ccccc45)n3)cc2)c2c(oc3c4c([2H])c([2H])c([2H])c([2H])c4c([2H])c([2H])c32)c1[2H] ; [2H]c1c([2H])c(-c2cccc(-c3nc(-c4cccc(-c5ccccc5)c4)nc(-c4ccc5c(c4)sc4ccc(-c6ccccc6)cc45)n3)c2)c2c(oc3c4c([2H])c([2H])c([2H])c([2H])c4c([2H])c([2H])c32)c1[2H]]

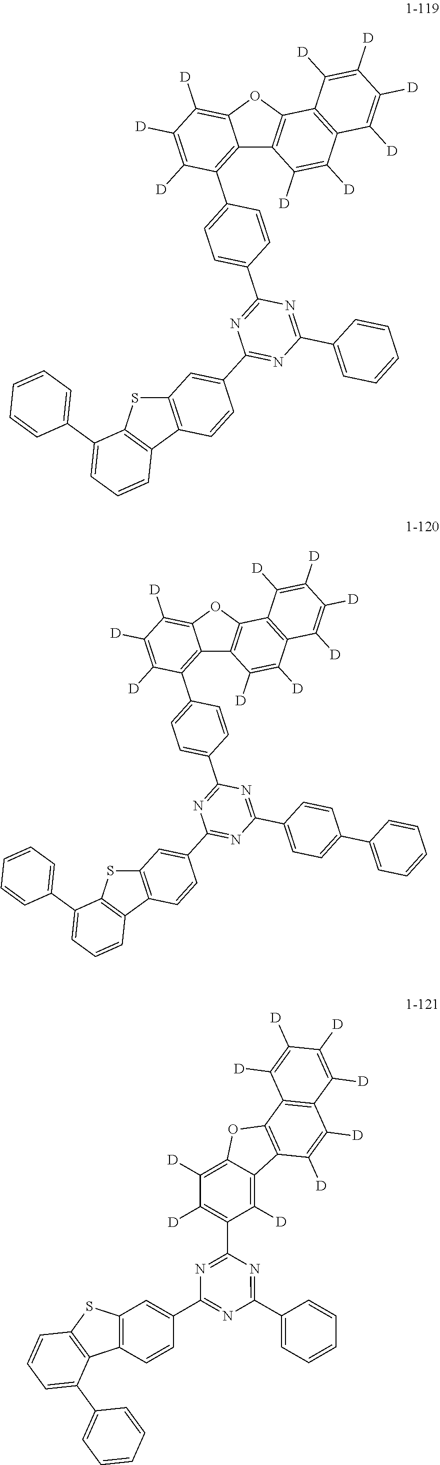 [SMILES: [2H]c1c([2H])c(-c2ccc(-c3nc(-c4ccccc4)nc(-c4ccc5c(c4)sc4c(-c6ccccc6)cccc45)n3)cc2)c2c(oc3c4c([2H])c([2H])c([2H])c([2H])c4c([2H])c([2H])c32)c1[2H] ; [2H]c1c([2H])c(-c2ccc(-c3nc(-c4ccc(-c5ccccc5)cc4)nc(-c4ccc5c(c4)sc4c(-c6ccccc6)cccc45)n3)cc2)c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]]

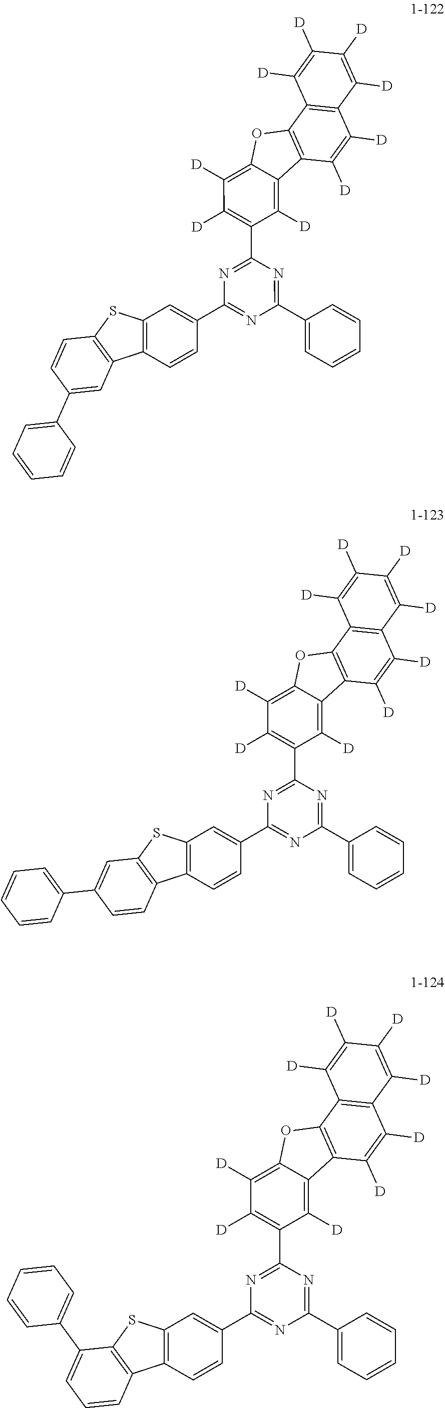 [SMILES: [2H]c1c(-c2nc(-c3ccccc3)nc(-c3ccc4c(c3)sc3ccc(-c5ccccc5)cc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)sc3cc(-c5ccccc5)ccc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc4c(c3)sc3c(-c5ccccc5)cccc34)n2)c([2H])c2c(oc3c4c([2H])c([2H])c([2H])c([2H])c4c([2H])c([2H])c32)c1[2H]]

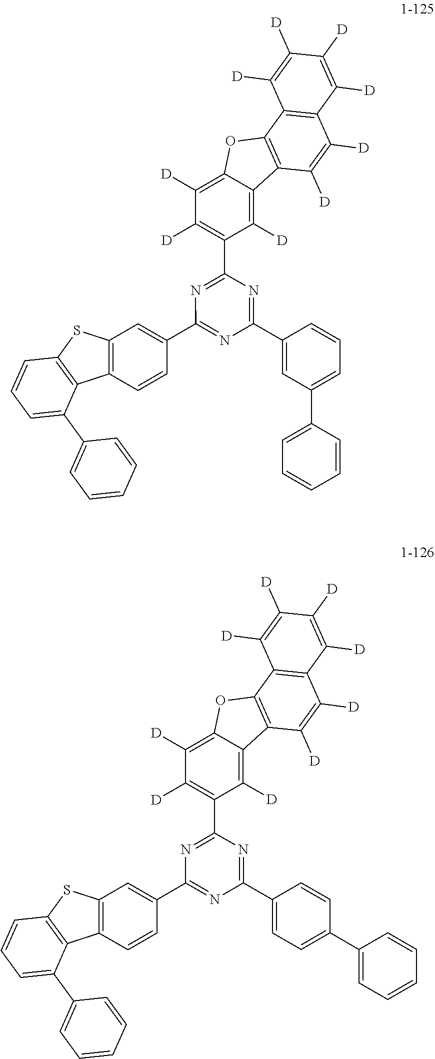 [SMILES: [2H]c1c(-c2nc(-c3cccc(-c4ccccc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3ccc(-c4ccccc4)cc3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]]

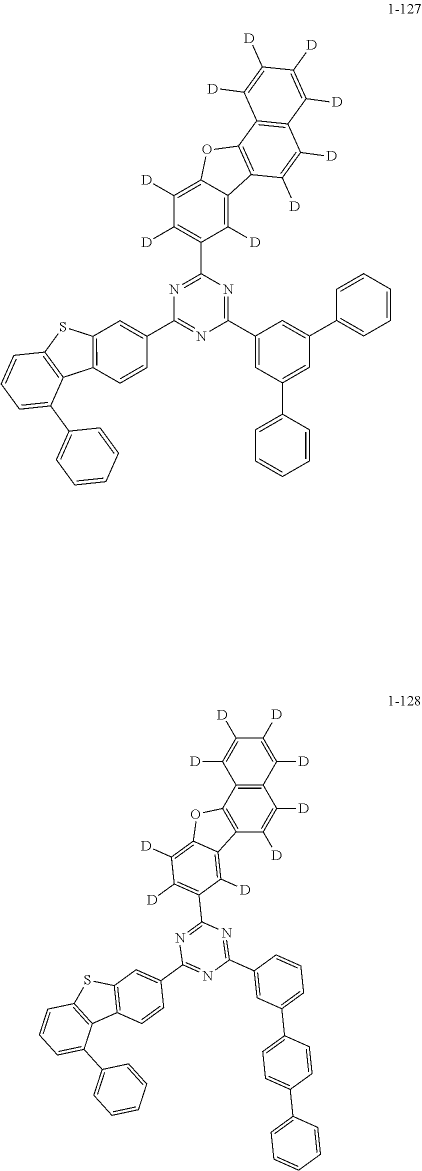 [SMILES: [2H]c1c(-c2nc(-c3cc(-c4ccccc4)cc(-c4ccccc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H] ; [2H]c1c(-c2nc(-c3cccc(-c4ccc(-c5ccccc5)cc4)c3)nc(-c3ccc4c(c3)sc3cccc(-c5ccccc5)c34)n2)c([2H])c2c(oc3c4c([2H])c([2H])c([2H])c([2H])c4c([2H])c([2H])c32)c1[2H]]

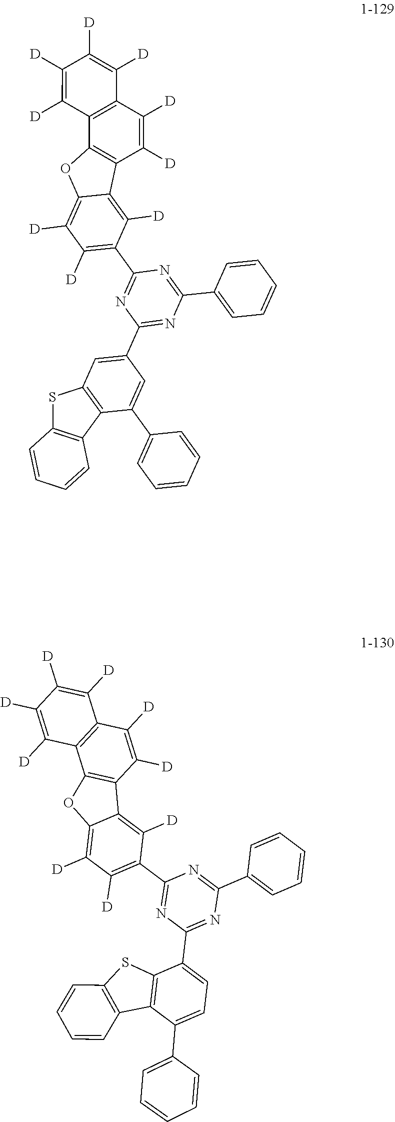 [SMILES: [2H]c1c(-c2nc(-c3ccccc3)nc(-c3cc(-c4ccccc4)c4c(c3)sc3ccccc34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc(-c4ccccc4)c4c3sc3ccccc34)n2)c([2H])c2c(oc3c4c([2H])c([2H])c([2H])c([2H])c4c([2H])c([2H])c32)c1[2H]]

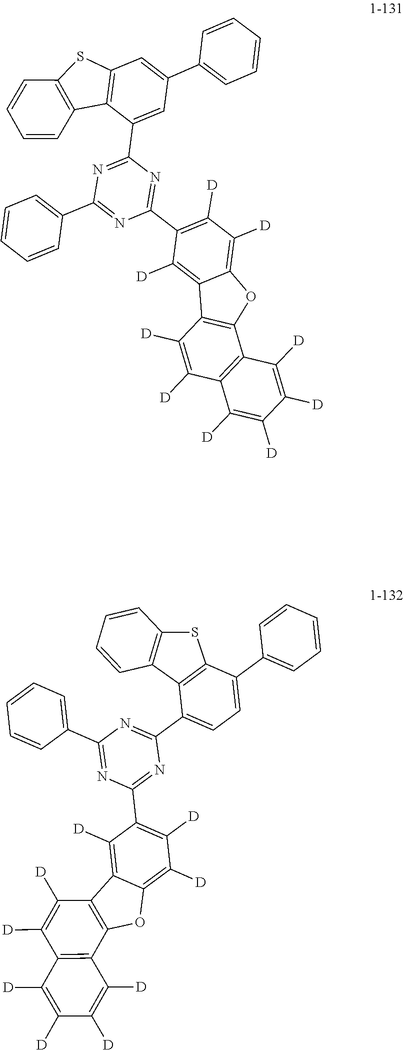 [SMILES: [2H]c1c(-c2nc(-c3ccccc3)nc(-c3cc(-c4ccccc4)cc4sc5ccccc5c34)n2)c([2H])c2c(oc3c4c([2H])c([2H])c([2H])c([2H])c4c([2H])c([2H])c32)c1[2H] ; [2H]c1c(-c2nc(-c3ccccc3)nc(-c3ccc(-c4ccccc4)c4sc5ccccc5c34)n2)c([2H])c2c(oc3c4c([2H])c([2H])c([2H])c([2H])c4c([2H])c([2H])c32)c1[2H]]

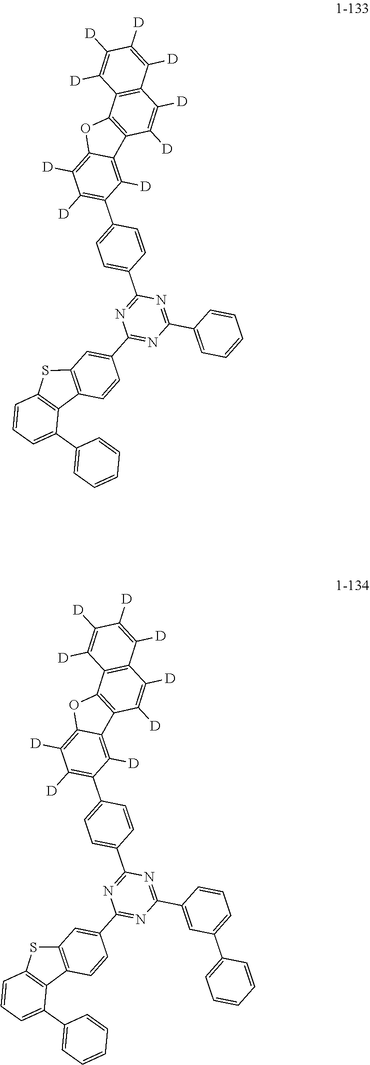 [SMILES: [2H]c1c(-c2ccc(-c3nc(-c4ccccc4)nc(-c4ccc5c(c4)sc4cccc(-c6ccccc6)c45)n3)cc2)c([2H])c2c(oc3c4c([2H])c([2H])c([2H])c([2H])c4c([2H])c([2H])c32)c1[2H] ; [2H]c1c(-c2ccc(-c3nc(-c4cccc(-c5ccccc5)c4)nc(-c4ccc5c(c4)sc4cccc(-c6ccccc6)c45)n3)cc2)c([2H])c2c(oc3c4c([2H])c([2H])c([2H])c([2H])c4c([2H])c([2H])c32)c1[2H]]

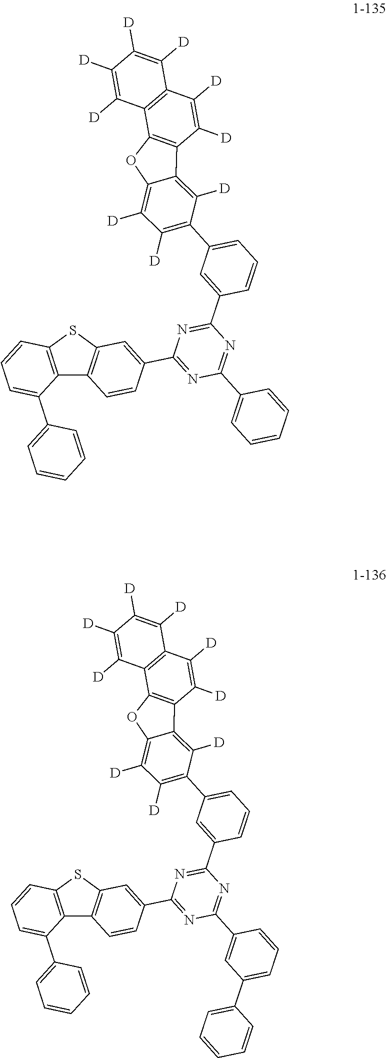 [SMILES: [2H]c1c(-c2cccc(-c3nc(-c4ccccc4)nc(-c4ccc5c(c4)sc4cccc(-c6ccccc6)c45)n3)c2)c([2H])c2c(oc3c4c([2H])c([2H])c([2H])c([2H])c4c([2H])c([2H])c32)c1[2H] ; [2H]c1c(-c2cccc(-c3nc(-c4cccc(-c5ccccc5)c4)nc(-c4ccc5c(c4)sc4cccc(-c6ccccc6)c45)n3)c2)c([2H])c2c(oc3c4c([2H])c([2H])c([2H])c([2H])c4c([2H])c([2H])c32)c1[2H]]

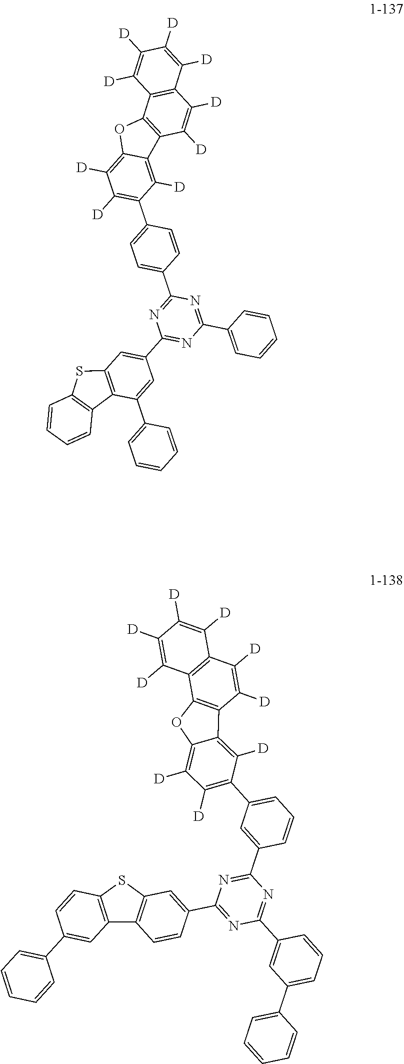 [SMILES: [2H]c1c(-c2ccc(-c3nc(-c4ccccc4)nc(-c4cc(-c5ccccc5)c5c(c4)sc4ccccc45)n3)cc2)c([2H])c2c(oc3c4c([2H])c([2H])c([2H])c([2H])c4c([2H])c([2H])c32)c1[2H] ; [2H]c1c(-c2cccc(-c3nc(-c4cccc(-c5ccccc5)c4)nc(-c4ccc5c(c4)sc4ccc(-c6ccccc6)cc45)n3)c2)c([2H])c2c(oc3c4c([2H])c([2H])c([2H])c([2H])c4c([2H])c([2H])c32)c1[2H]]

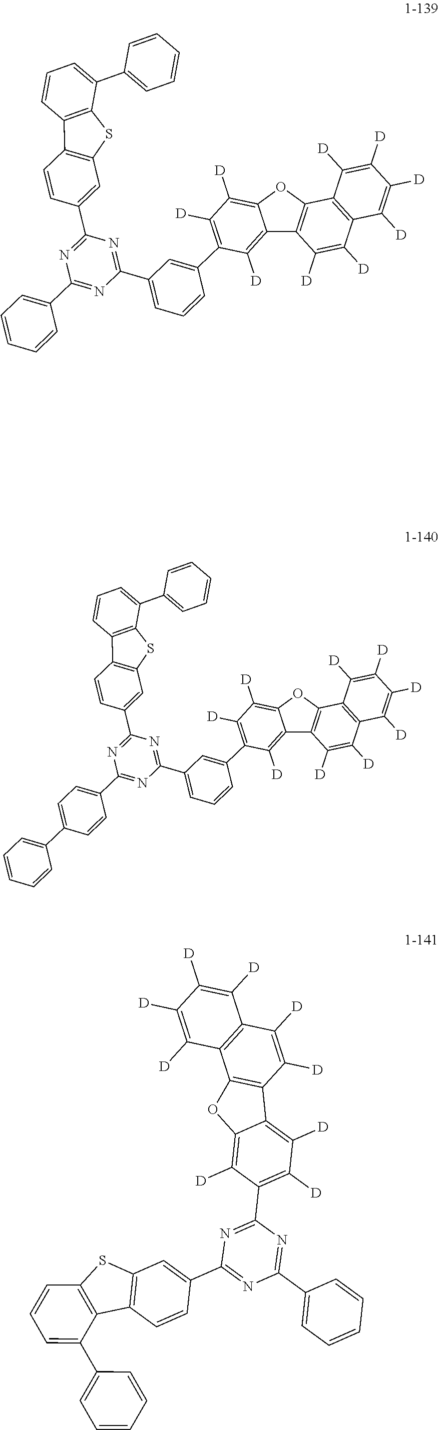 [SMILES: [2H]c1c(-c2cccc(-c3nc(-c4ccccc4)nc(-c4ccc5c(c4)sc4c(-c6ccccc6)cccc45)n3)c2)c([2H])c2c(oc3c4c([2H])c([2H])c([2H])c([2H])c4c([2H])c([2H])c32)c1[2H] ; [2H]c1c(-c2cccc(-c3nc(-c4ccc(-c5ccccc5)cc4)nc(-c4ccc5c(c4)sc4c(-c6ccccc6)cccc45)n3)c2)c([2H])c2c(oc3c4c([2H])c([2H])c([2H])c([2H])c4c([2H])c([2H])c32)c1[2H] ; [2H]c1c([2H])c([2H])c2c(c1[2H])c([2H])c([2H])c1c2oc2c([2H])c(-c3nc(-c4ccccc4)nc(-c4ccc5c(c4)sc4cccc(-c6ccccc6)c45)n3)c([2H])c([2H])c21]

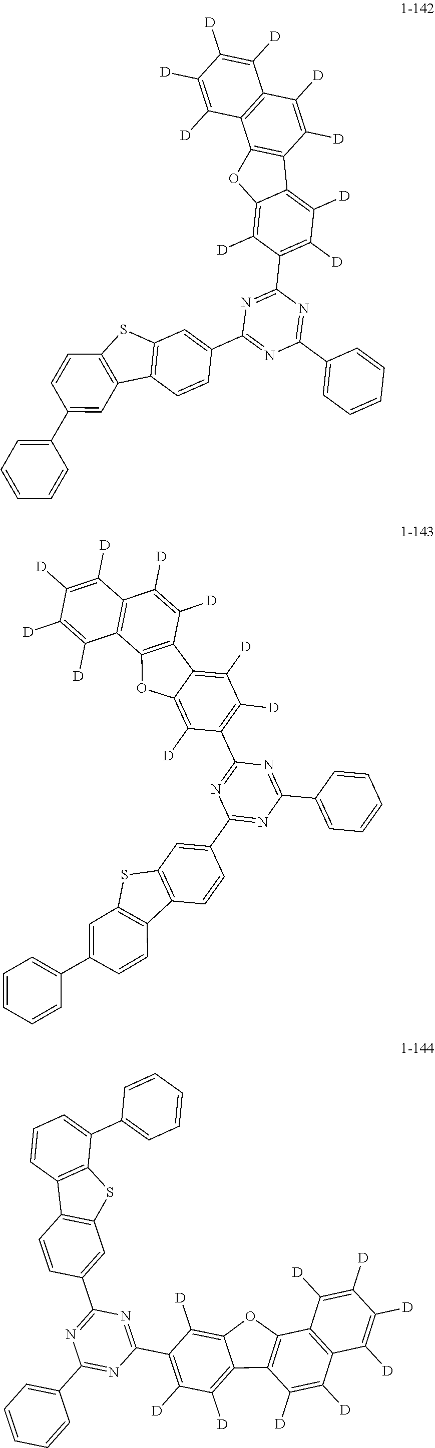 [SMILES: [2H]c1c([2H])c([2H])c2c(c1[2H])c([2H])c([2H])c1c2oc2c([2H])c(-c3nc(-c4ccccc4)nc(-c4ccc5c(c4)sc4ccc(-c6ccccc6)cc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)sc4cc(-c6ccccc6)ccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc5c(c4)sc4c(-c6ccccc6)cccc45)n3)c([2H])c([2H])c21]

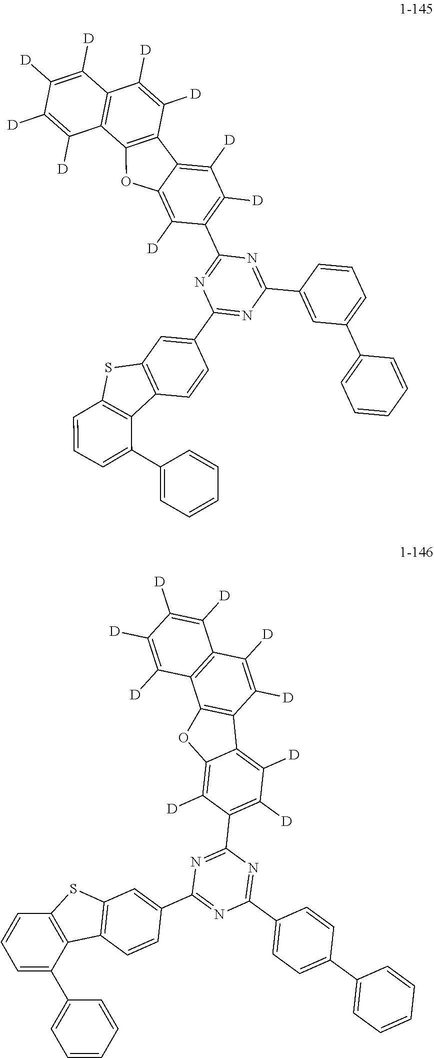 [SMILES: [2H]c1c([2H])c([2H])c2c(c1[2H])c([2H])c([2H])c1c2oc2c([2H])c(-c3nc(-c4cccc(-c5ccccc5)c4)nc(-c4ccc5c(c4)sc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4ccc(-c5ccccc5)cc4)nc(-c4ccc5c(c4)sc4cccc(-c6ccccc6)c45)n3)c([2H])c([2H])c21]

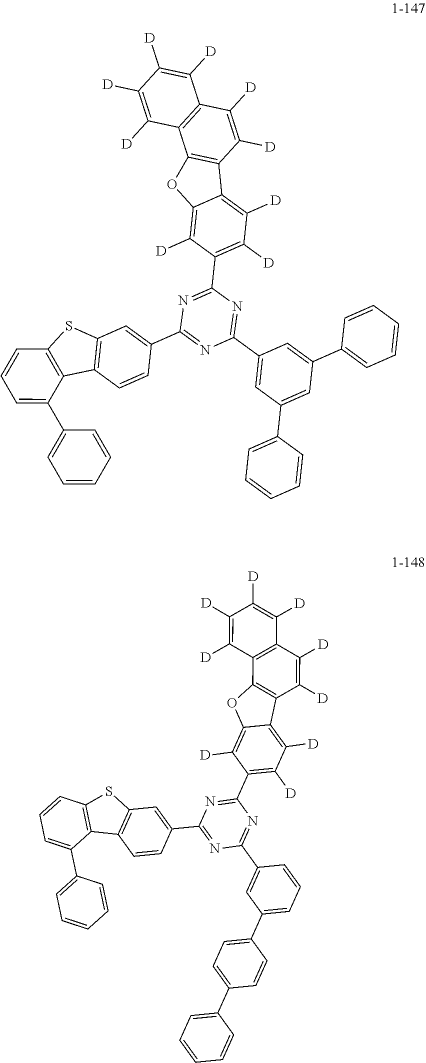 [SMILES: [2H]c1c([2H])c([2H])c2c(c1[2H])c([2H])c([2H])c1c2oc2c([2H])c(-c3nc(-c4cc(-c5ccccc5)cc(-c5ccccc5)c4)nc(-c4ccc5c(c4)sc4cccc(-c6ccccc6)c45)n3)c([2H])c([2H])c21 ; [2H]c1c([2H])c([2H])c2c(c1[2H])c([2H])c([2H])c1c2oc2c([2H])c(-c3nc(-c4cccc(-c5ccc(-c6ccccc6)cc5)c4)nc(-c4ccc5c(c4)sc4cccc(-c6ccccc6)c45)n3)c([2H])c([2H])c21]

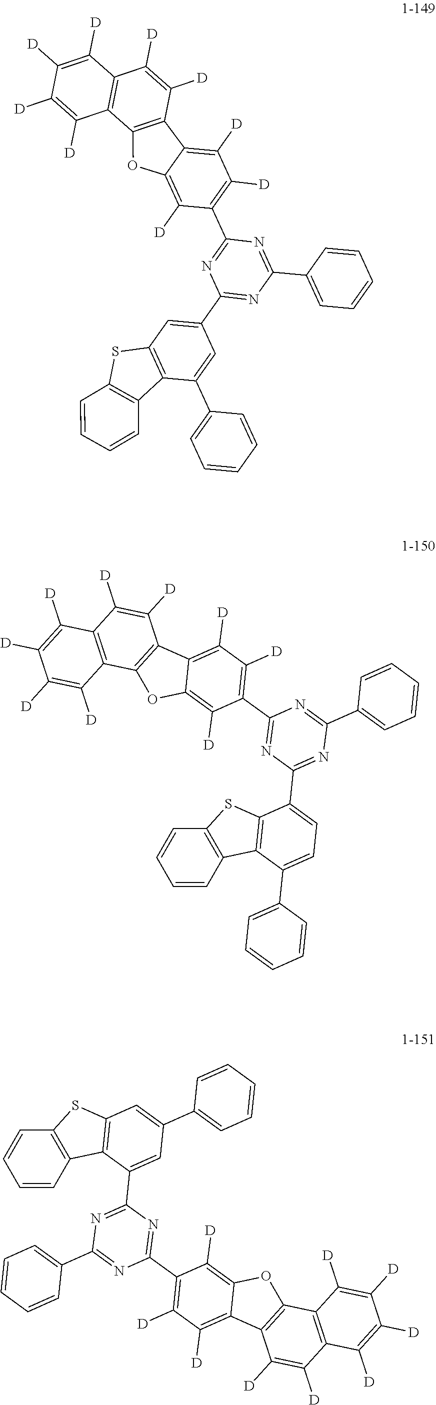 [SMILES: [2H]c1c([2H])c([2H])c2c(c1[2H])c([2H])c([2H])c1c2oc2c([2H])c(-c3nc(-c4ccccc4)nc(-c4cc(-c5ccccc5)c5c(c4)sc4ccccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4ccc(-c5ccccc5)c5c4sc4ccccc45)n3)c([2H])c([2H])c21 ; [2H]c1c([2H])c([2H])c2c(c1[2H])c([2H])c([2H])c1c2oc2c([2H])c(-c3nc(-c4ccccc4)nc(-c4cc(-c5ccccc5)cc5sc6ccccc6c45)n3)c([2H])c([2H])c21]

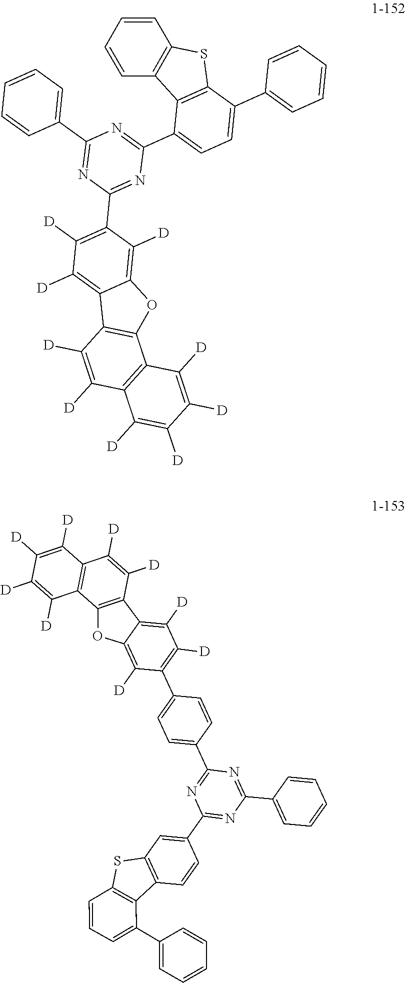 [SMILES: [2H]c1c([2H])c([2H])c2c(c1[2H])c([2H])c([2H])c1c2oc2c([2H])c(-c3nc(-c4ccccc4)nc(-c4ccc(-c5ccccc5)c5sc6ccccc6c45)n3)c([2H])c([2H])c21 ; [2H]c1c([2H])c([2H])c2c(c1[2H])c([2H])c([2H])c1c2oc2c([2H])c(-c3ccc(-c4nc(-c5ccccc5)nc(-c5ccc6c(c5)sc5cccc(-c7ccccc7)c56)n4)cc3)c([2H])c([2H])c21]

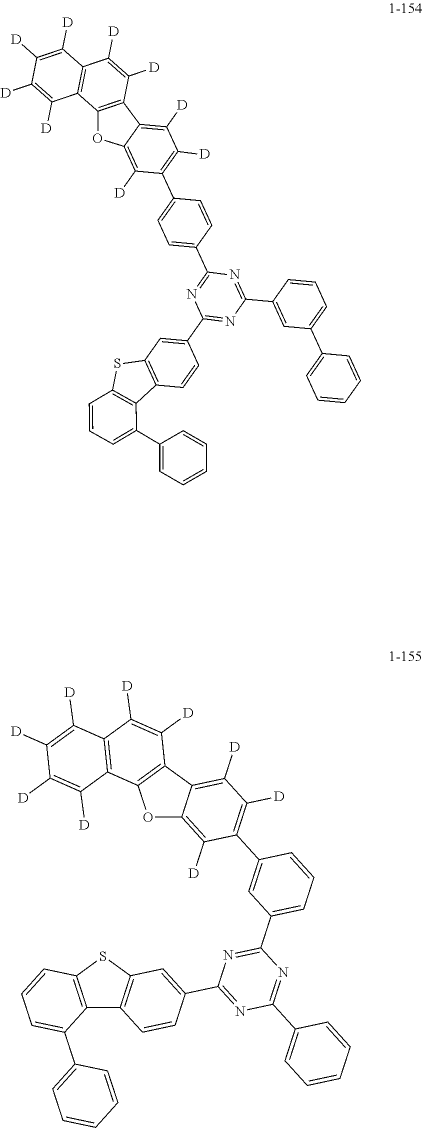 [SMILES: [2H]c1c([2H])c([2H])c2c(c1[2H])c([2H])c([2H])c1c2oc2c([2H])c(-c3ccc(-c4nc(-c5cccc(-c6ccccc6)c5)nc(-c5ccc6c(c5)sc5cccc(-c7ccccc7)c56)n4)cc3)c([2H])c([2H])c21 ; [2H]c1c([2H])c([2H])c2c(c1[2H])c([2H])c([2H])c1c2oc2c([2H])c(-c3cccc(-c4nc(-c5ccccc5)nc(-c5ccc6c(c5)sc5cccc(-c7ccccc7)c56)n4)c3)c([2H])c([2H])c21]

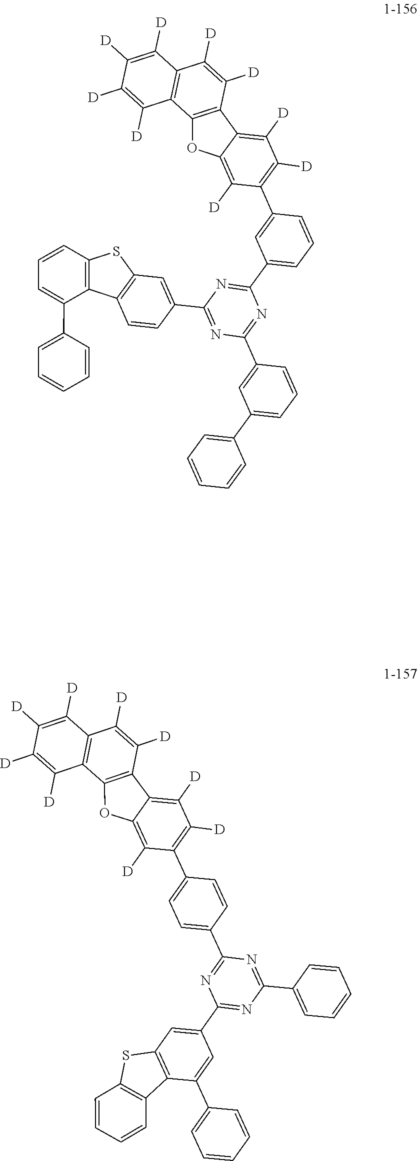 [SMILES: [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)sc5cccc(-c7ccccc7)c56)n4)c3)c([2H])c([2H])c21 ; [2H]c1c([2H])c([2H])c2c(c1[2H])c([2H])c([2H])c1c2oc2c([2H])c(-c3ccc(-c4nc(-c5ccccc5)nc(-c5cc(-c6ccccc6)c6c(c5)sc5ccccc56)n4)cc3)c([2H])c([2H])c21]

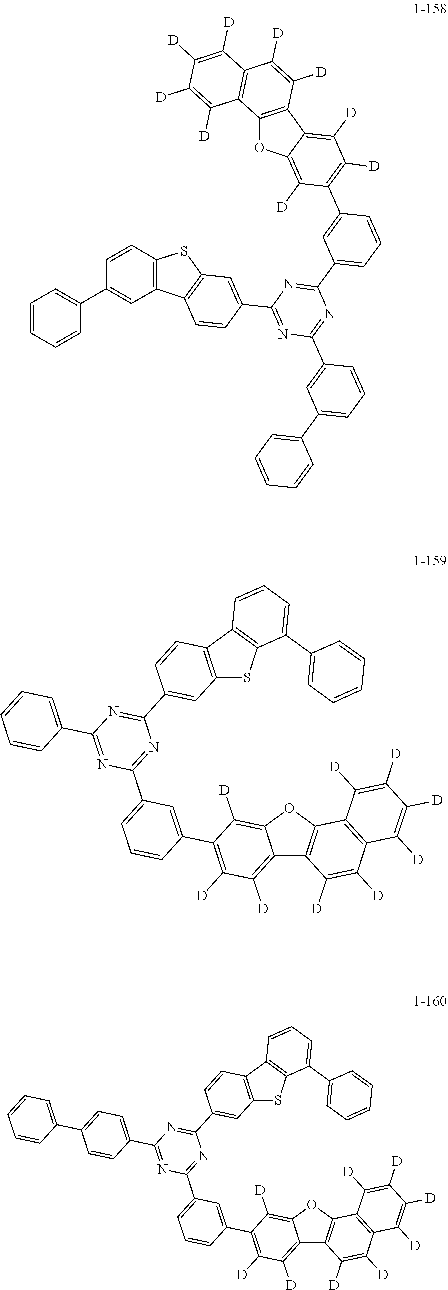 [SMILES: [2H]c1c([2H])c([2H])c2c(c1[2H])c([2H])c([2H])c1c2oc2c([2H])c(-c3cccc(-c4nc(-c5cccc(-c6ccccc6)c5)nc(-c5ccc6c(c5)sc5ccc(-c7ccccc7)cc56)n4)c3)c([2H])c([2H])c21 ; [2H]c1c([2H])c([2H])c2c(c1[2H])c([2H])c([2H])c1c2oc2c([2H])c(-c3cccc(-c4nc(-c5ccccc5)nc(-c5ccc6c(c5)sc5c(-c7ccccc7)cccc56)n4)c3)c([2H])c([2H])c21 ; [2H]c1c([2H])c([2H])c2c(c1[2H])c([2H])c([2H])c1c2oc2c([2H])c(-c3cccc(-c4nc(-c5ccc(-c6ccccc6)cc5)nc(-c5ccc6c(c5)sc5c(-c7ccccc7)cccc56)n4)c3)c([2H])c([2H])c21]

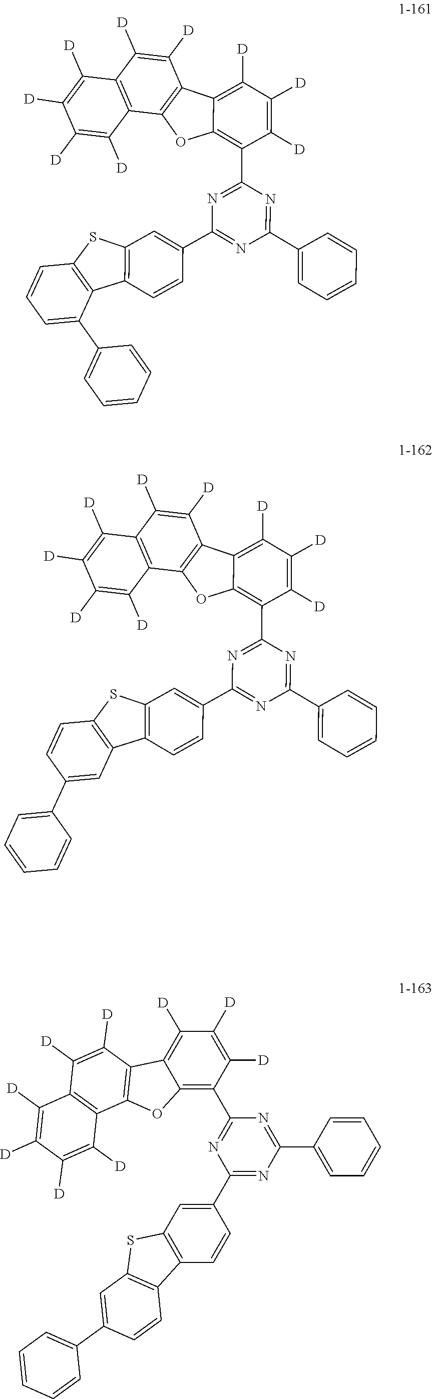 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2ccc(-c4ccccc4)cc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2cc(-c4ccccc4)ccc23)n1]

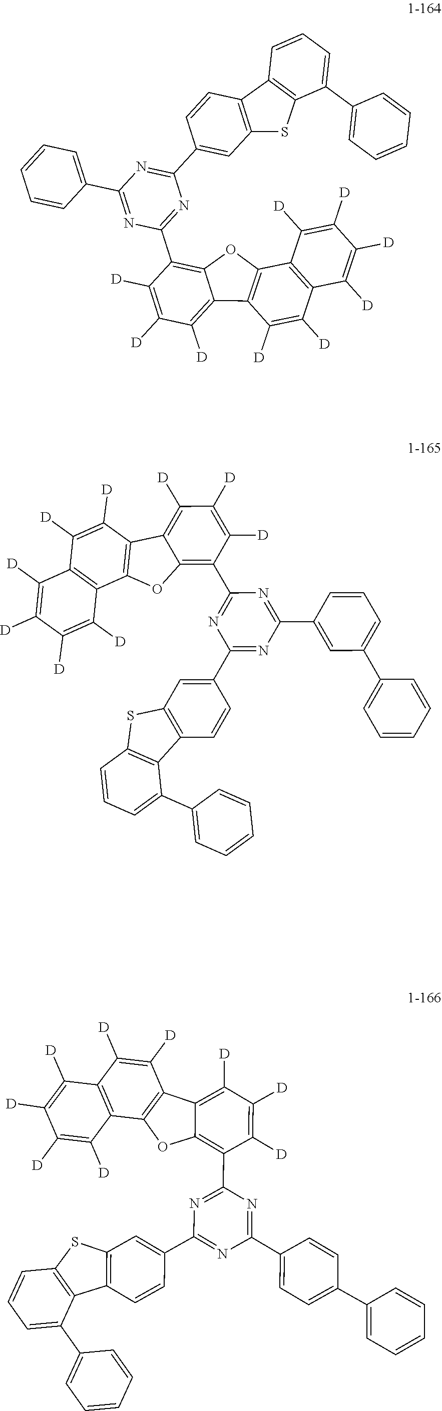 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc3c(c2)sc2c(-c4ccccc4)cccc23)n1 ; [2H]c1c([2H])c([2H])c2c(oc3c4c([2H])c([2H])c([2H])c([2H])c4c([2H])c([2H])c32)c1-c1nc(-c2cccc(-c3ccccc3)c2)nc(-c2ccc3c(c2)sc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccc(-c3ccccc3)cc2)nc(-c2ccc3c(c2)sc2cccc(-c4ccccc4)c23)n1]

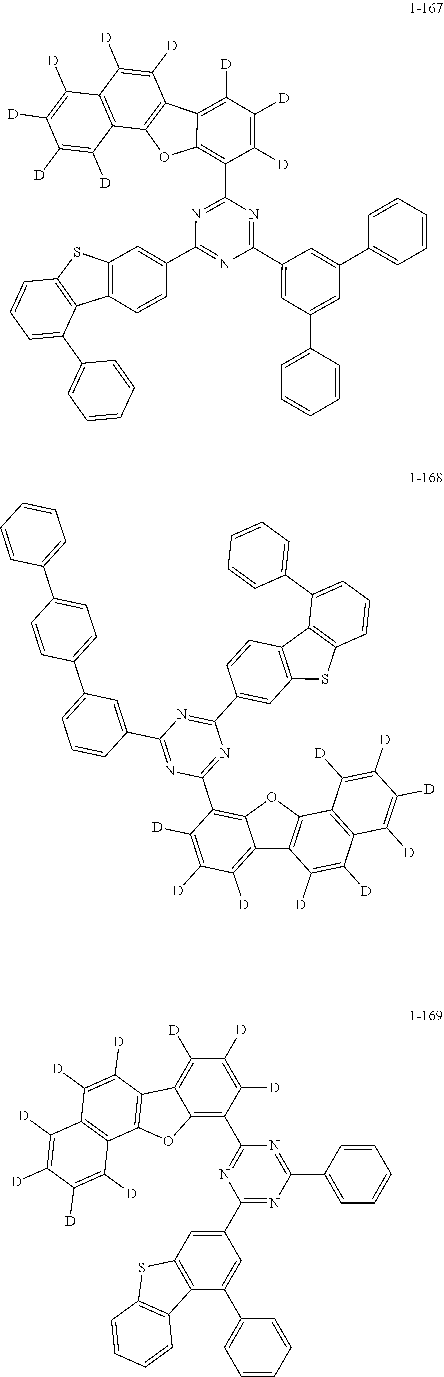 [SMILES: [2H]c1c([2H])c([2H])c2c(oc3c4c([2H])c([2H])c([2H])c([2H])c4c([2H])c([2H])c32)c1-c1nc(-c2cc(-c3ccccc3)cc(-c3ccccc3)c2)nc(-c2ccc3c(c2)sc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2cccc(-c3ccc(-c4ccccc4)cc3)c2)nc(-c2ccc3c(c2)sc2cccc(-c4ccccc4)c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)c3c(c2)sc2ccccc23)n1]

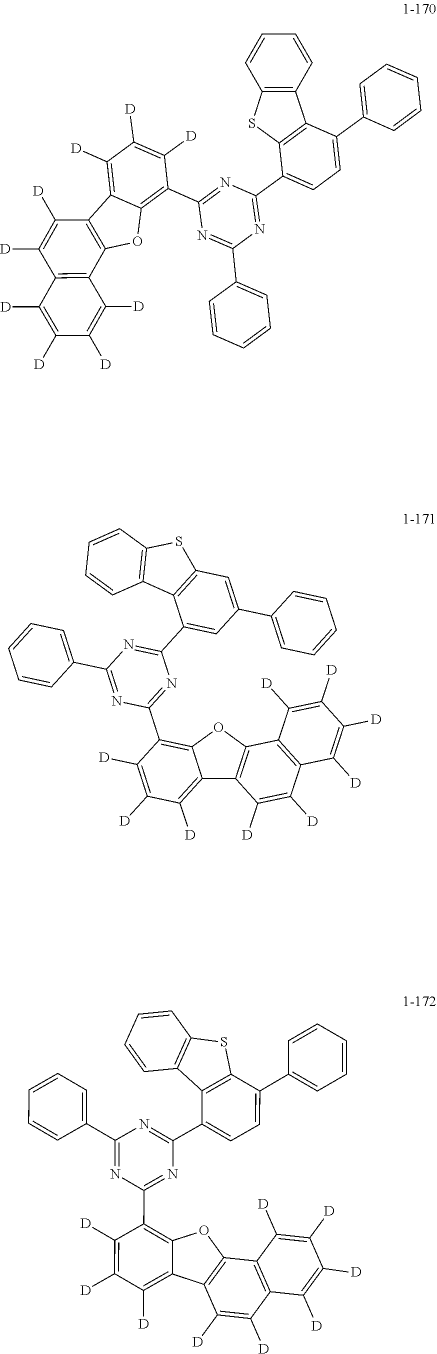 [SMILES: [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3c2sc2ccccc23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2cc(-c3ccccc3)cc3sc4ccccc4c23)n1 ; [2H]c1c([2H])c([2H])c2c(oc3c4c([2H])c([2H])c([2H])c([2H])c4c([2H])c([2H])c32)c1-c1nc(-c2ccccc2)nc(-c2ccc(-c3ccccc3)c3sc4ccccc4c23)n1]

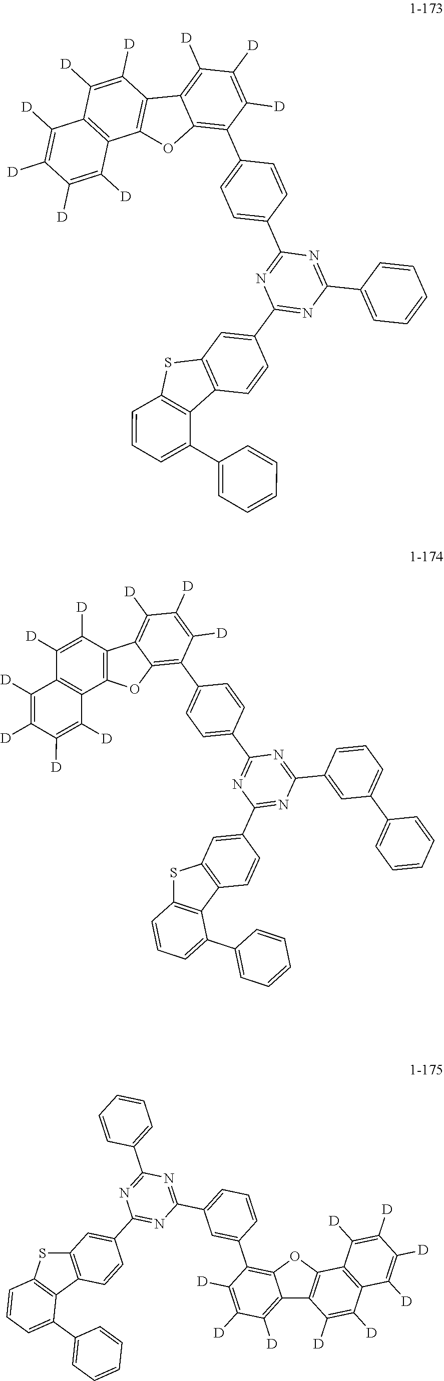 [SMILES: [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3ccc4c(c3)sc3cccc(-c5ccccc5)c34)n2)cc1 ; [2H]c1c([2H])c([2H])c2c(oc3c4c([2H])c([2H])c([2H])c([2H])c4c([2H])c([2H])c32)c1-c1ccc(-c2nc(-c3cccc(-c4ccccc4)c3)nc(-c3ccc4c(c3)sc3cccc(-c5ccccc5)c34)n2)cc1 ; [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)sc3cccc(-c5ccccc5)c34)n2)c1]

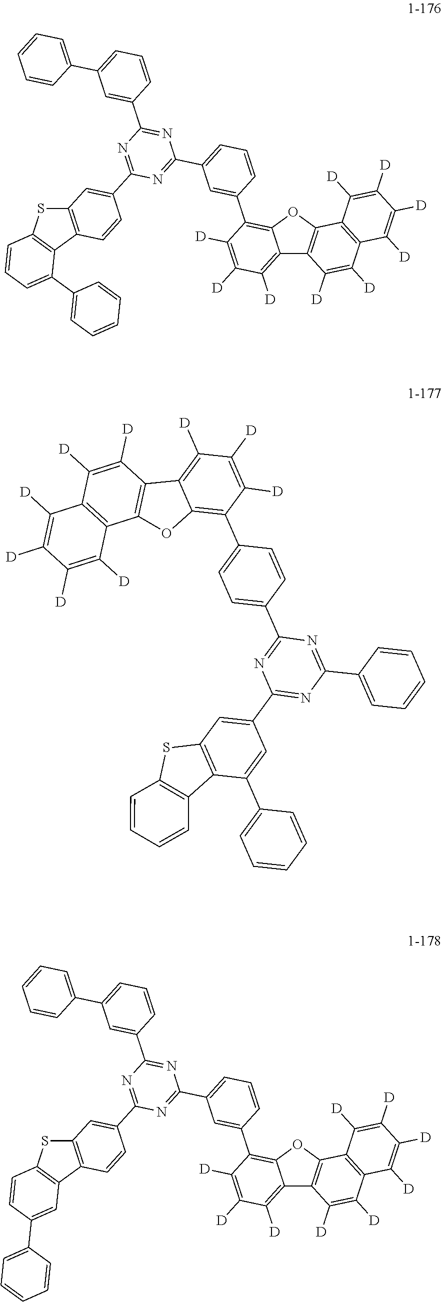 [SMILES: [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3cccc(-c5ccccc5)c34)n2)c1 ; [2H]c1c([2H])c([2H])c2c(oc3c4c([2H])c([2H])c([2H])c([2H])c4c([2H])c([2H])c32)c1-c1ccc(-c2nc(-c3ccccc3)nc(-c3cc(-c4ccccc4)c4c(c3)sc3ccccc34)n2)cc1 ; [2H]c1c([2H])c([2H])c2c(oc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3ccc(-c5ccccc5)cc34)n2)c1]

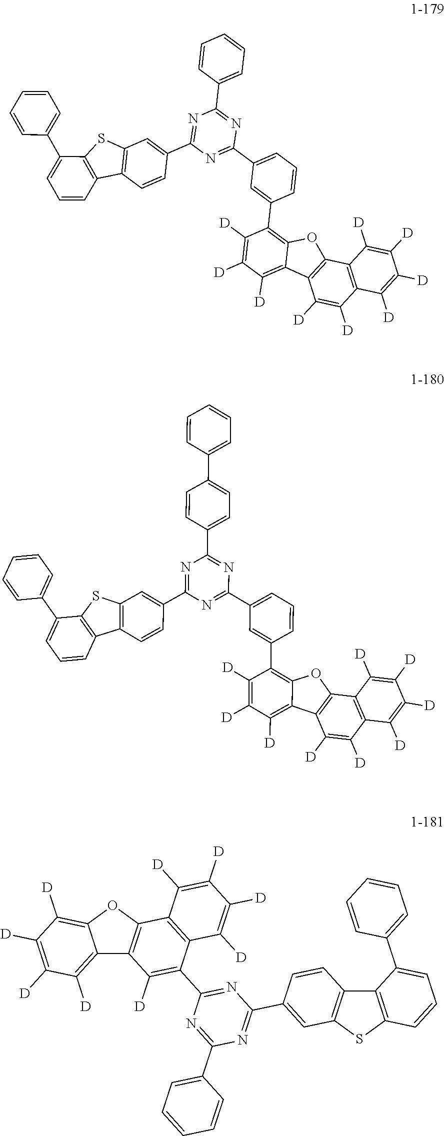 [SMILES: [2H]c1c([2H])c([2H])c2c(oc3c4c([2H])c([2H])c([2H])c([2H])c4c([2H])c([2H])c32)c1-c1cccc(-c2nc(-c3ccccc3)nc(-c3ccc4c(c3)sc3c(-c5ccccc5)cccc34)n2)c1 ; [2H]c1c([2H])c([2H])c2c(oc3c4c([2H])c([2H])c([2H])c([2H])c4c([2H])c([2H])c32)c1-c1cccc(-c2nc(-c3ccc(-c4ccccc4)cc3)nc(-c3ccc4c(c3)sc3c(-c5ccccc5)cccc34)n2)c1 ; [2H]c1c([2H])c([2H])c2c(oc3c4c([2H])c([2H])c([2H])c([2H])c4c(-c4nc(-c5ccccc5)nc(-c5ccc6c(c5)sc5cccc(-c7ccccc7)c56)n4)c([2H])c32)c1[2H]]

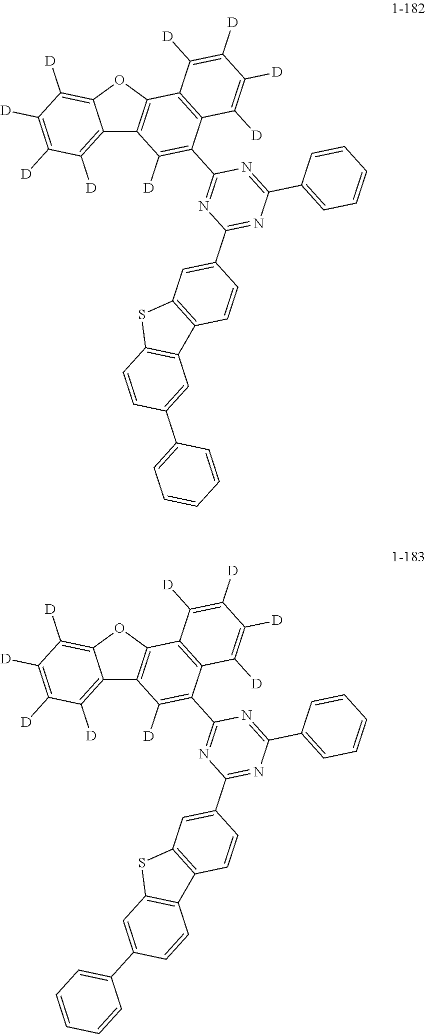 [SMILES: [2H]c1c([2H])c([2H])c2c(oc3c4c([2H])c([2H])c([2H])c([2H])c4c(-c4nc(-c5ccccc5)nc(-c5ccc6c(c5)sc5ccc(-c7ccccc7)cc56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc6c(c5)sc5cc(-c7ccccc7)ccc56)n4)c([2H])c32)c1[2H]]

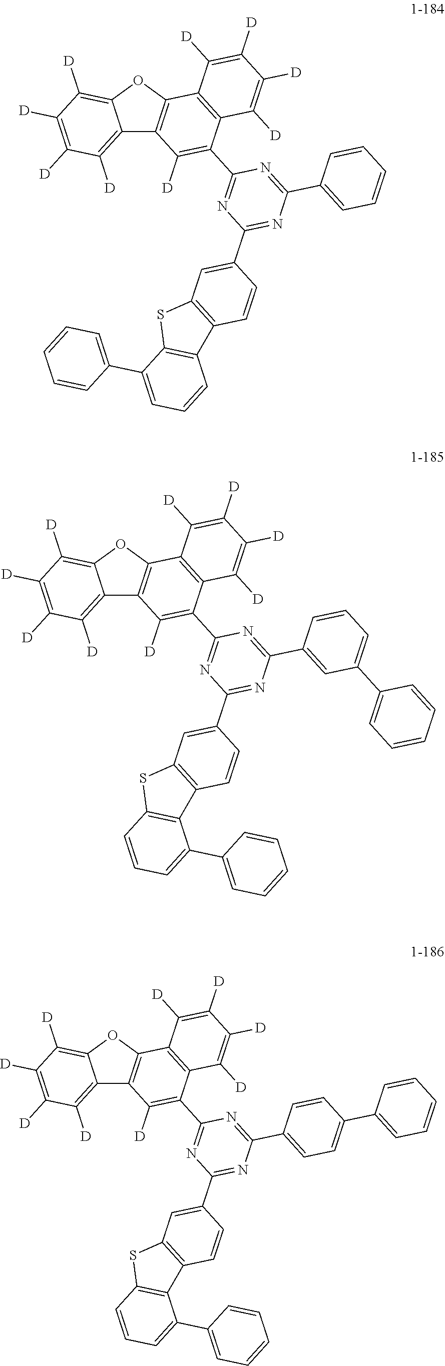 [SMILES: [2H]c1c([2H])c([2H])c2c(oc3c4c([2H])c([2H])c([2H])c([2H])c4c(-c4nc(-c5ccccc5)nc(-c5ccc6c(c5)sc5c(-c7ccccc7)cccc56)n4)c([2H])c32)c1[2H] ; [2H]c1c([2H])c([2H])c2c(oc3c4c([2H])c([2H])c([2H])c([2H])c4c(-c4nc(-c5cccc(-c6ccccc6)c5)nc(-c5ccc6c(c5)sc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5ccc(-c6ccccc6)cc5)nc(-c5ccc6c(c5)sc5cccc(-c7ccccc7)c56)n4)c([2H])c32)c1[2H]]

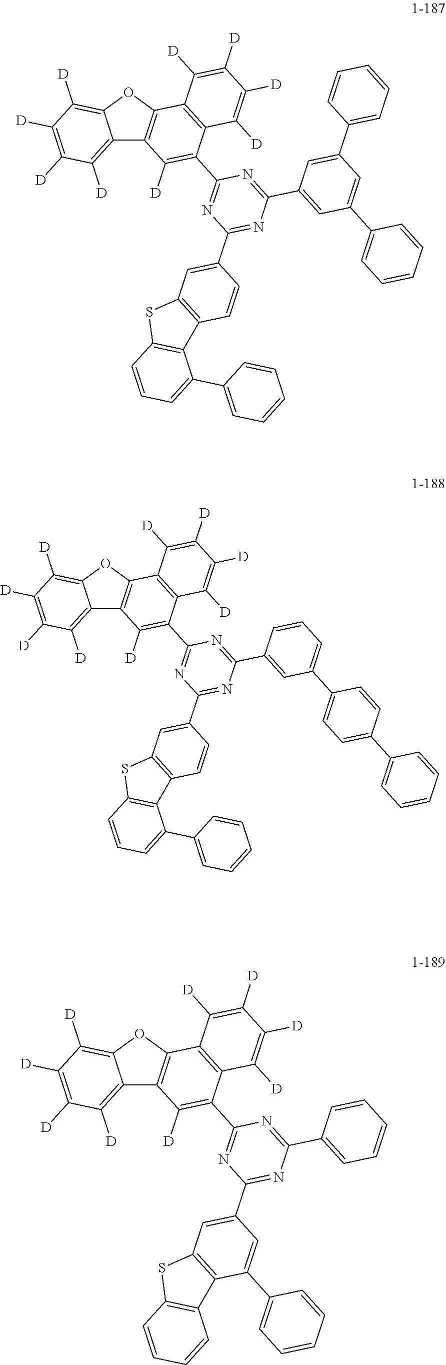 [SMILES: [2H]c1c([2H])c([2H])c2c(oc3c4c([2H])c([2H])c([2H])c([2H])c4c(-c4nc(-c5cc(-c6ccccc6)cc(-c6ccccc6)c5)nc(-c5ccc6c(c5)sc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5cccc(-c6ccc(-c7ccccc7)cc6)c5)nc(-c5ccc6c(c5)sc5cccc(-c7ccccc7)c56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5cc(-c6ccccc6)c6c(c5)sc5ccccc56)n4)c([2H])c32)c1[2H]]

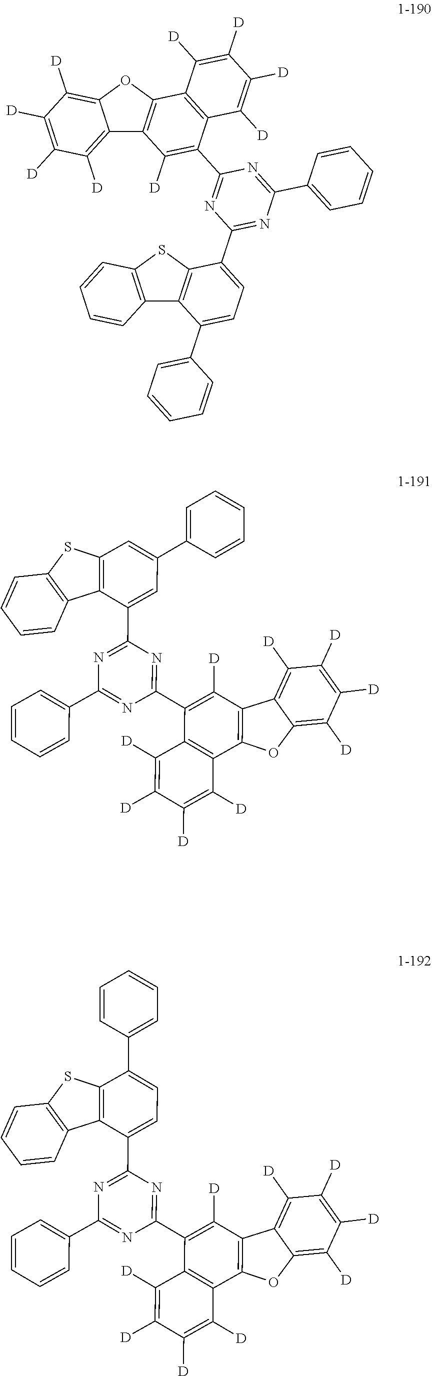 [SMILES: [2H]c1c([2H])c([2H])c2c(oc3c4c([2H])c([2H])c([2H])c([2H])c4c(-c4nc(-c5ccccc5)nc(-c5ccc(-c6ccccc6)c6c5sc5ccccc56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5cc(-c6ccccc6)cc6sc7ccccc7c56)n4)c([2H])c32)c1[2H] ; [2H]c1c([2H])c([2H])c2c(oc3c4c([2H])c([2H])c([2H])c([2H])c4c(-c4nc(-c5ccccc5)nc(-c5ccc(-c6ccccc6)c6sc7ccccc7c56)n4)c([2H])c32)c1[2H]]

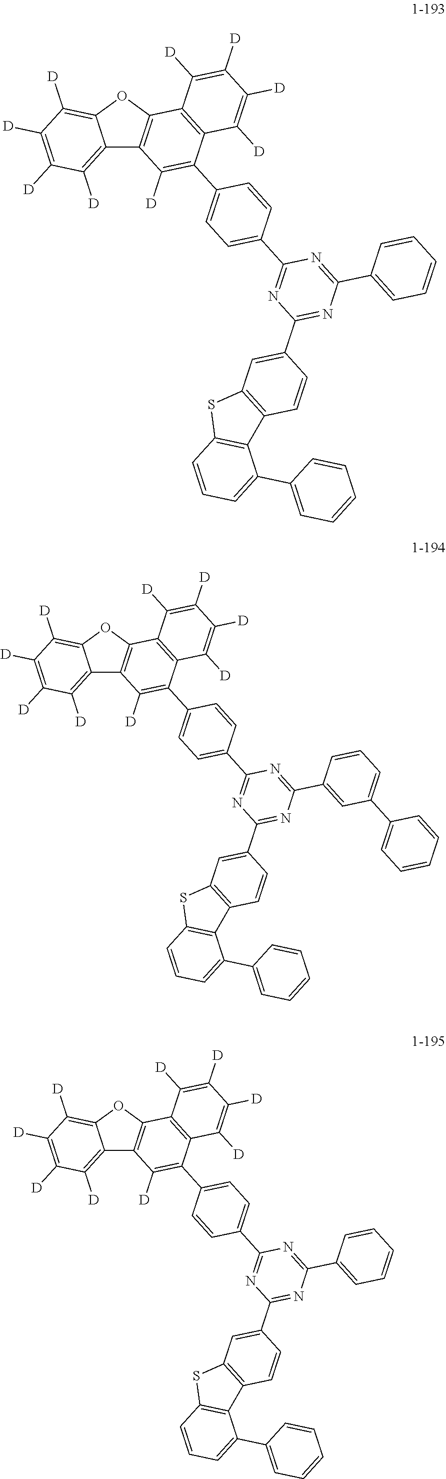 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)sc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)sc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6ccccc6)nc(-c6ccc7c(c6)sc6cccc(-c8ccccc8)c67)n5)cc4)c([2H])c32)c1[2H]]

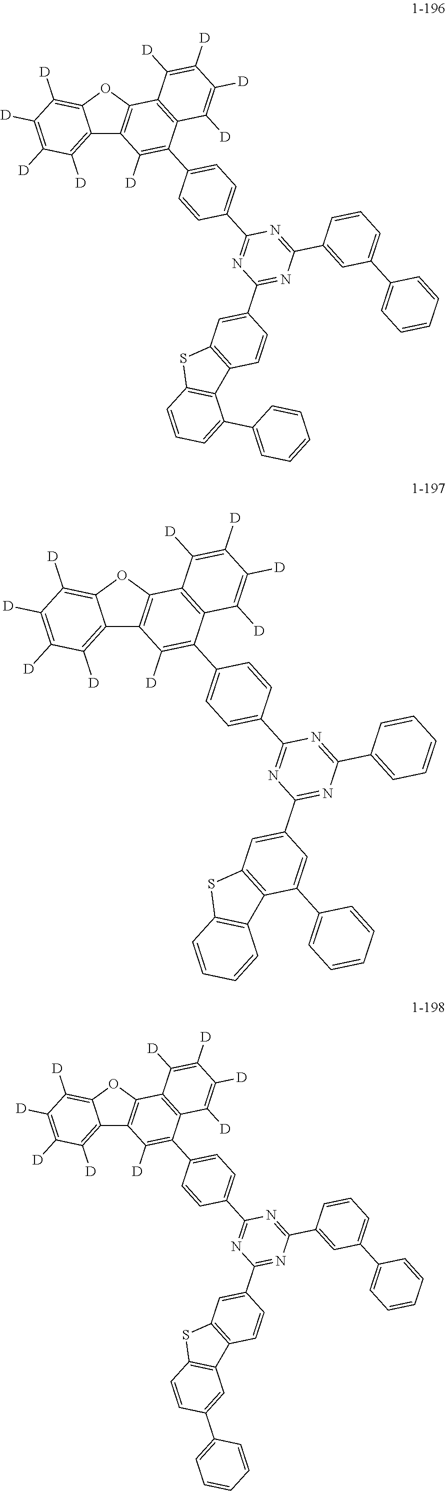 [SMILES: [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)sc6cccc(-c8ccccc8)c67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6ccccc6)nc(-c6cc(-c7ccccc7)c7c(c6)sc6ccccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6cccc(-c7ccccc7)c6)nc(-c6ccc7c(c6)sc6ccc(-c8ccccc8)cc67)n5)cc4)c([2H])c32)c1[2H]]

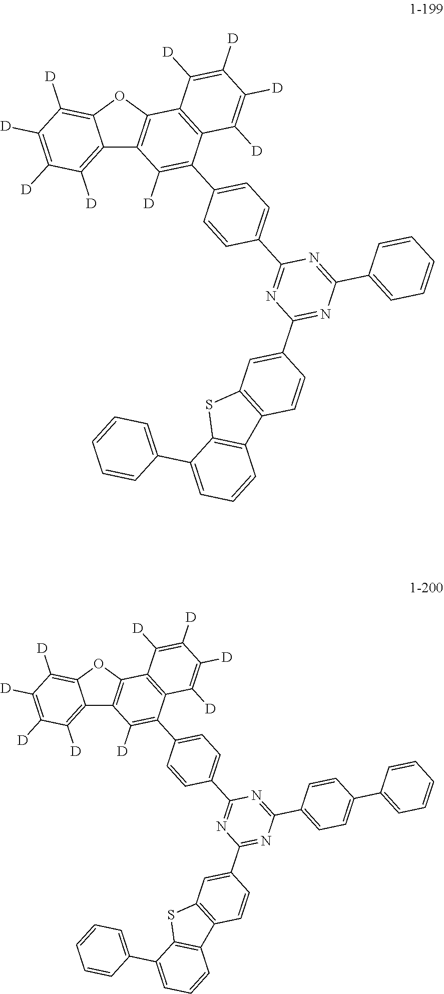 [SMILES: [2H]c1c([2H])c([2H])c2c(oc3c4c([2H])c([2H])c([2H])c([2H])c4c(-c4ccc(-c5nc(-c6ccccc6)nc(-c6ccc7c(c6)sc6c(-c8ccccc8)cccc67)n5)cc4)c([2H])c32)c1[2H] ; [2H]c1c([2H])c([2H])c2c(oc3c4c([2H])c([2H])c([2H])c([2H])c4c(-c4ccc(-c5nc(-c6ccc(-c7ccccc7)cc6)nc(-c6ccc7c(c6)sc6c(-c8ccccc8)cccc67)n5)cc4)c([2H])c32)c1[2H]]

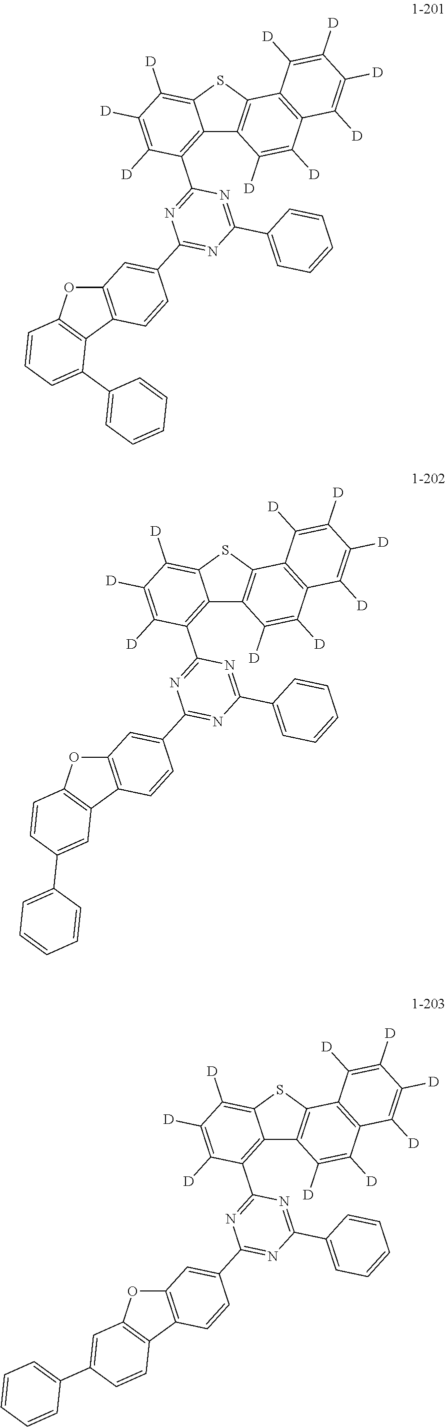 [SMILES: [2H]c1c([2H])c(-c2nc(-c3ccccc3)nc(-c3ccc4c(c3)oc3cccc(-c5ccccc5)c34)n2)c2c(sc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3ccc(-c5ccccc5)cc34)n2)c2c(sc3c4c([2H])c([2H])c([2H])c([2H])c4c([2H])c([2H])c32)c1[2H] ; [2H]c1c([2H])c(-c2nc(-c3ccccc3)nc(-c3ccc4c(c3)oc3cc(-c5ccccc5)ccc34)n2)c2c(sc3c4c([2H])c([2H])c([2H])c([2H])c4c([2H])c([2H])c32)c1[2H]]

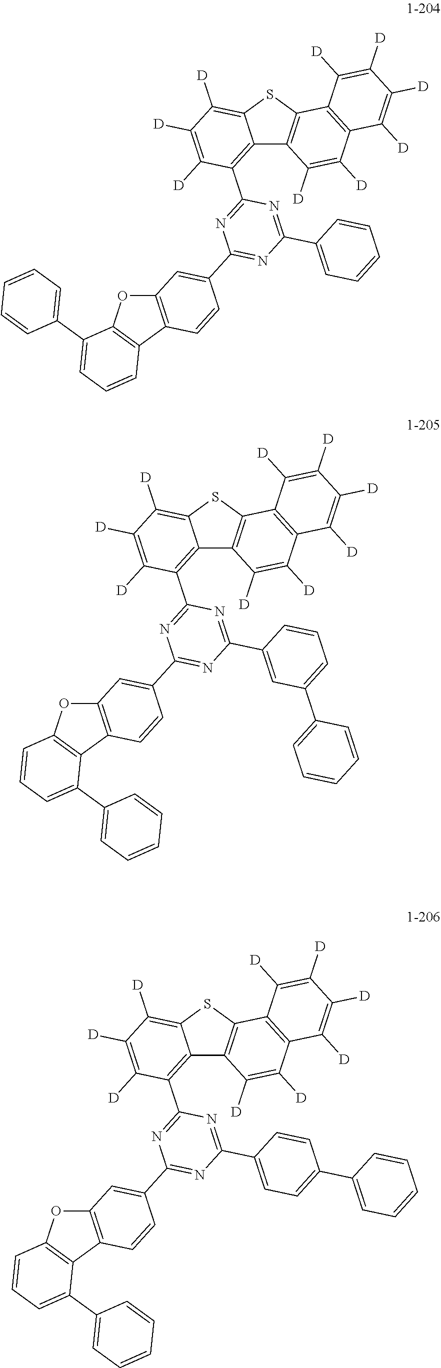 [SMILES: [2H]c1c([2H])c(-c2nc(-c3ccccc3)nc(-c3ccc4c(c3)oc3c(-c5ccccc5)cccc34)n2)c2c(sc3c4c([2H])c([2H])c([2H])c([2H])c4c([2H])c([2H])c32)c1[2H] ; [2H]c1c([2H])c(-c2nc(-c3cccc(-c4ccccc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H] ; [2H]c1c([2H])c(-c2nc(-c3ccc(-c4ccccc4)cc3)nc(-c3ccc4c(c3)oc3cccc(-c5ccccc5)c34)n2)c2c(sc3c4c([2H])c([2H])c([2H])c([2H])c4c([2H])c([2H])c32)c1[2H]]

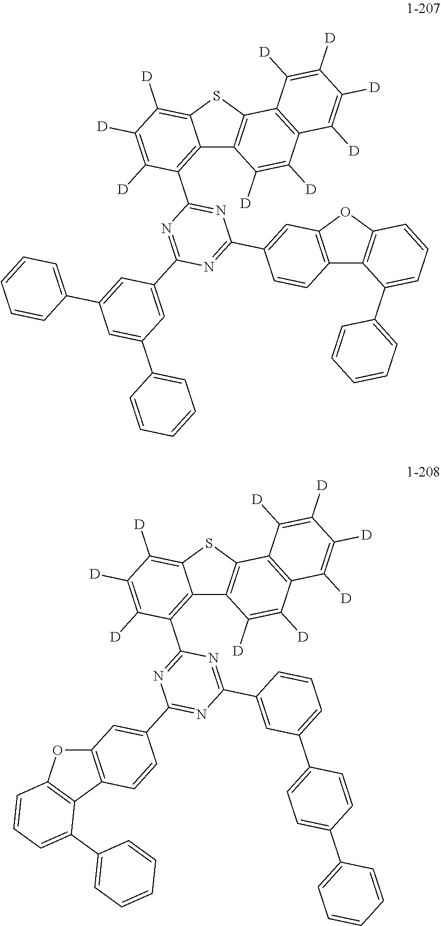 [SMILES: [2H]c1c([2H])c(-c2nc(-c3cc(-c4ccccc4)cc(-c4ccccc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H] ; [2H]c1c([2H])c(-c2nc(-c3cccc(-c4ccc(-c5ccccc5)cc4)c3)nc(-c3ccc4c(c3)oc3cccc(-c5ccccc5)c34)n2)c2c(sc3c4c([2H])c([2H])c([2H])c([2H])c4c([2H])c([2H])c32)c1[2H]]

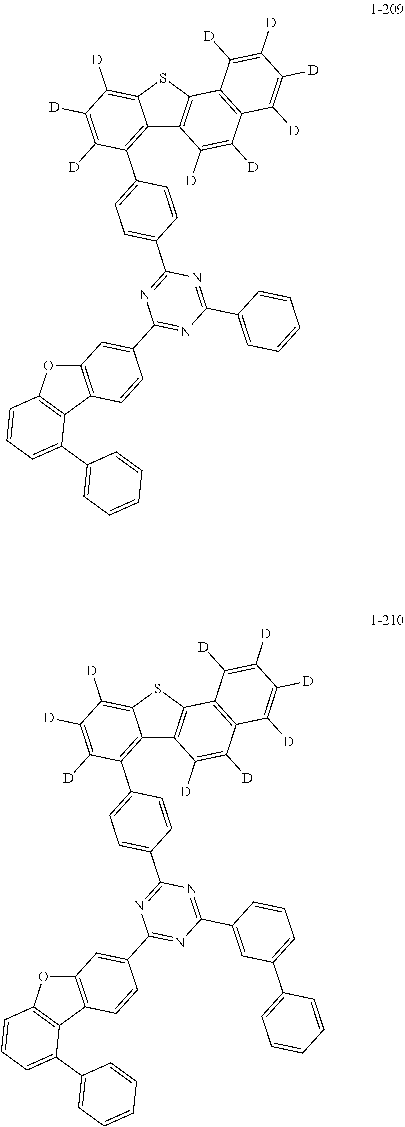 [SMILES: [2H]c1c([2H])c(-c2ccc(-c3nc(-c4ccccc4)nc(-c4ccc5c(c4)oc4cccc(-c6ccccc6)c45)n3)cc2)c2c(sc3c4c([2H])c([2H])c([2H])c([2H])c4c([2H])c([2H])c32)c1[2H] ; [2H]c1c([2H])c(-c2ccc(-c3nc(-c4cccc(-c5ccccc5)c4)nc(-c4ccc5c(c4)oc4cccc(-c6ccccc6)c45)n3)cc2)c2c(sc3c4c([2H])c([2H])c([2H])c([2H])c4c([2H])c([2H])c32)c1[2H]]

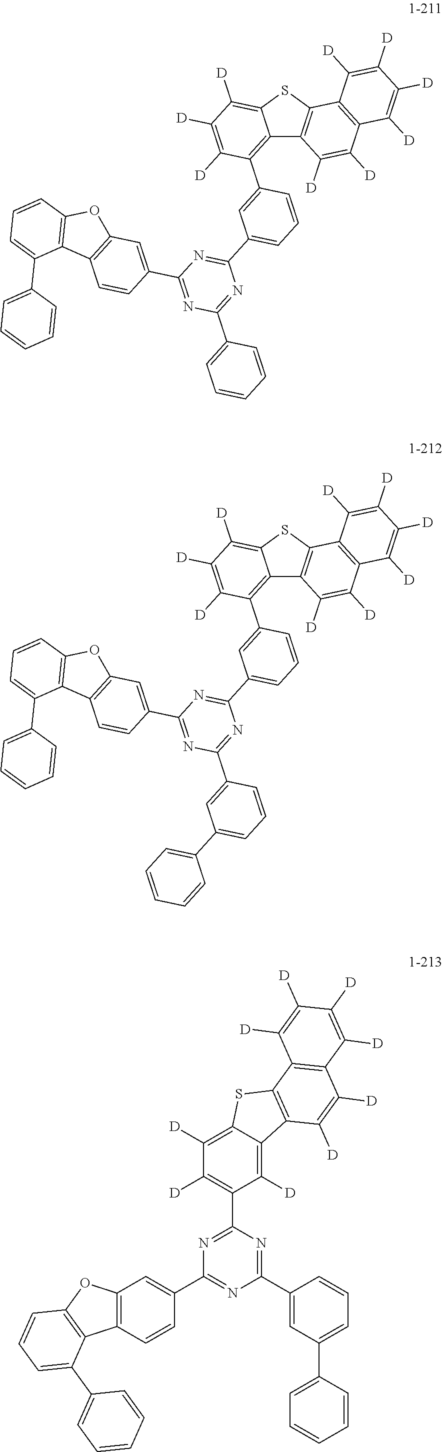 [SMILES: [2H]c1c([2H])c(-c2cccc(-c3nc(-c4ccccc4)nc(-c4ccc5c(c4)oc4cccc(-c6ccccc6)c45)n3)c2)c2c(sc3c4c([2H])c([2H])c([2H])c([2H])c4c([2H])c([2H])c32)c1[2H] ; [2H]c1c([2H])c(-c2cccc(-c3nc(-c4cccc(-c5ccccc5)c4)nc(-c4ccc5c(c4)oc4cccc(-c6ccccc6)c45)n3)c2)c2c(sc3c4c([2H])c([2H])c([2H])c([2H])c4c([2H])c([2H])c32)c1[2H] ; [2H]c1c(-c2nc(-c3cccc(-c4ccccc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H]]

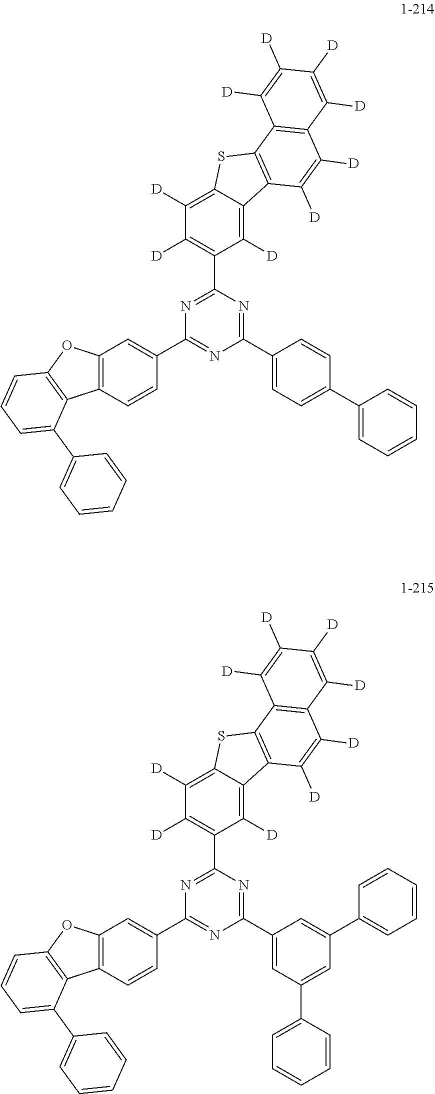 [SMILES: [2H]c1c(-c2nc(-c3ccc(-c4ccccc4)cc3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H] ; [2H]c1c(-c2nc(-c3cc(-c4ccccc4)cc(-c4ccccc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H]]

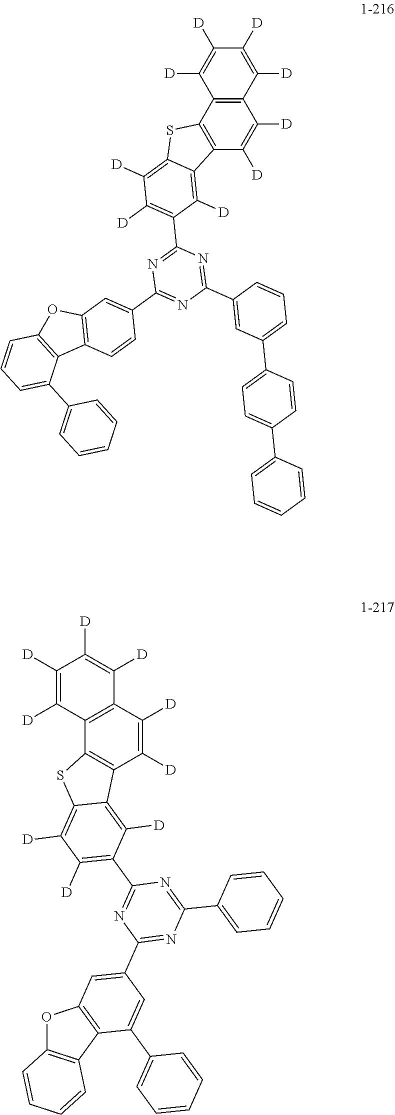 [SMILES: [2H]c1c(-c2nc(-c3cccc(-c4ccc(-c5ccccc5)cc4)c3)nc(-c3ccc4c(c3)oc3cccc(-c5ccccc5)c34)n2)c([2H])c2c(sc3c4c([2H])c([2H])c([2H])c([2H])c4c([2H])c([2H])c32)c1[2H] ; [2H]c1c(-c2nc(-c3ccccc3)nc(-c3cc(-c4ccccc4)c4c(c3)oc3ccccc34)n2)c([2H])c2c(sc3c4c([2H])c([2H])c([2H])c([2H])c4c([2H])c([2H])c32)c1[2H]]

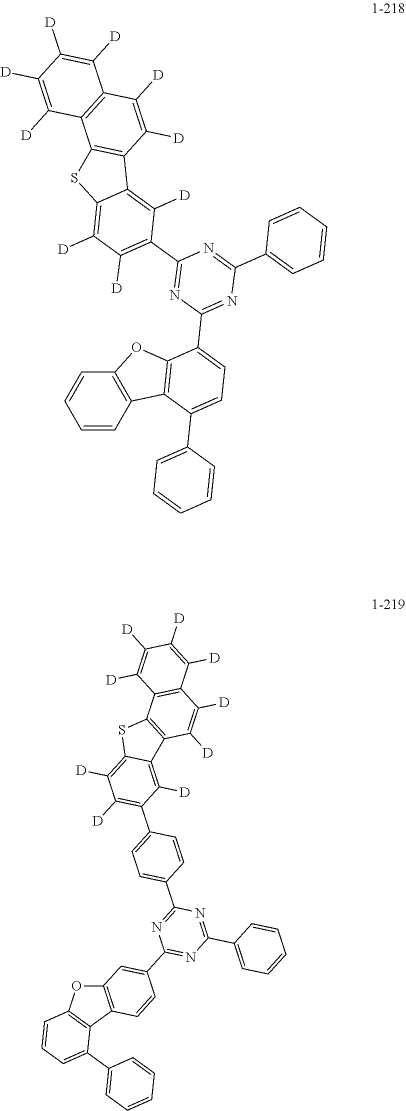 [SMILES: [2H]c1c(-c2nc(-c3ccccc3)nc(-c3ccc(-c4ccccc4)c4c3oc3ccccc34)n2)c([2H])c2c(sc3c4c([2H])c([2H])c([2H])c([2H])c4c([2H])c([2H])c32)c1[2H] ; [2H]c1c(-c2ccc(-c3nc(-c4ccccc4)nc(-c4ccc5c(c4)oc4cccc(-c6ccccc6)c45)n3)cc2)c([2H])c2c(sc3c4c([2H])c([2H])c([2H])c([2H])c4c([2H])c([2H])c32)c1[2H]]

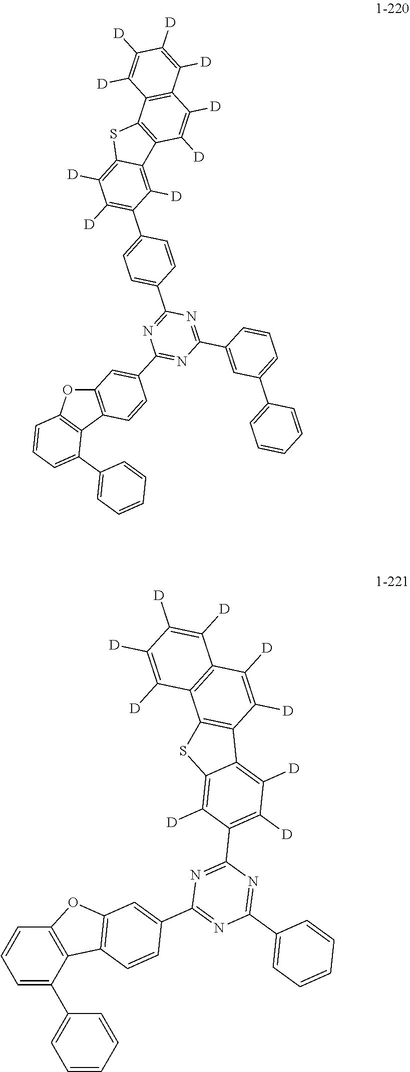 [SMILES: [2H]c1c(-c2ccc(-c3nc(-c4cccc(-c5ccccc5)c4)nc(-c4ccc5c(c4)oc4cccc(-c6ccccc6)c45)n3)cc2)c([2H])c2c(sc3c4c([2H])c([2H])c([2H])c([2H])c4c([2H])c([2H])c32)c1[2H] ; [2H]c1c([2H])c([2H])c2c(c1[2H])c([2H])c([2H])c1c2sc2c([2H])c(-c3nc(-c4ccccc4)nc(-c4ccc5c(c4)oc4cccc(-c6ccccc6)c45)n3)c([2H])c([2H])c21]

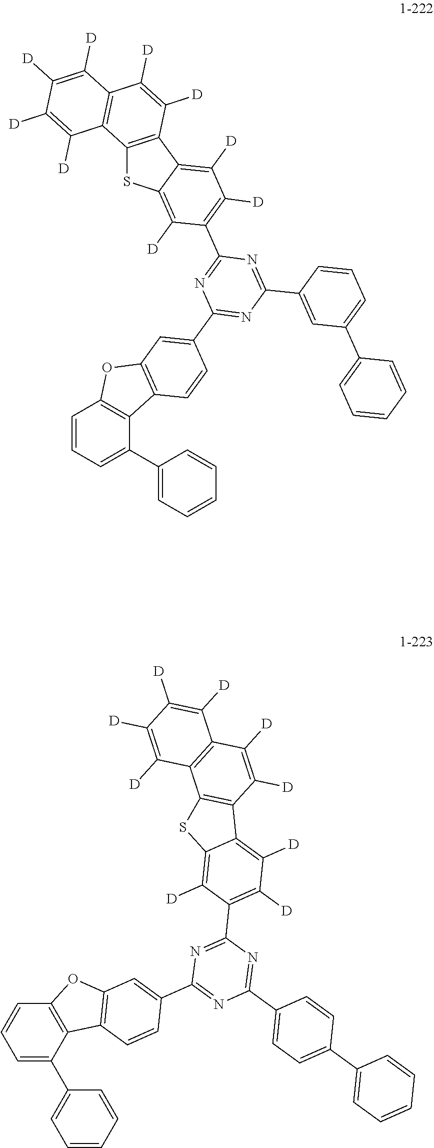 [SMILES: [2H]c1c([2H])c([2H])c2c(c1[2H])c([2H])c([2H])c1c2sc2c([2H])c(-c3nc(-c4cccc(-c5ccccc5)c4)nc(-c4ccc5c(c4)oc4cccc(-c6ccccc6)c45)n3)c([2H])c([2H])c21 ; [2H]c1c([2H])c([2H])c2c(c1[2H])c([2H])c([2H])c1c2sc2c([2H])c(-c3nc(-c4ccc(-c5ccccc5)cc4)nc(-c4ccc5c(c4)oc4cccc(-c6ccccc6)c45)n3)c([2H])c([2H])c21]

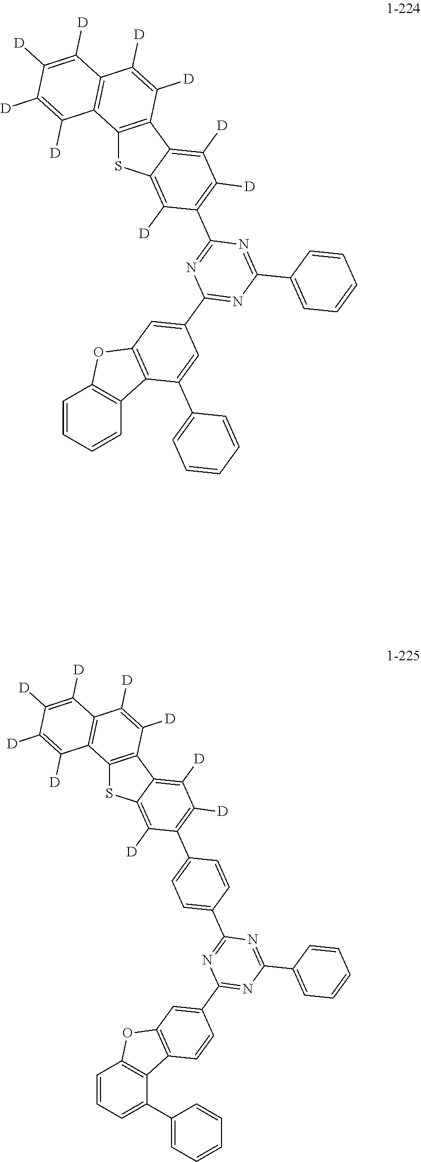 [SMILES: [2H]c1c([2H])c([2H])c2c(c1[2H])c([2H])c([2H])c1c2sc2c([2H])c(-c3nc(-c4ccccc4)nc(-c4cc(-c5ccccc5)c5c(c4)oc4ccccc45)n3)c([2H])c([2H])c21 ; [2H]c1c([2H])c([2H])c2c(c1[2H])c([2H])c([2H])c1c2sc2c([2H])c(-c3ccc(-c4nc(-c5ccccc5)nc(-c5ccc6c(c5)oc5cccc(-c7ccccc7)c56)n4)cc3)c([2H])c([2H])c21]

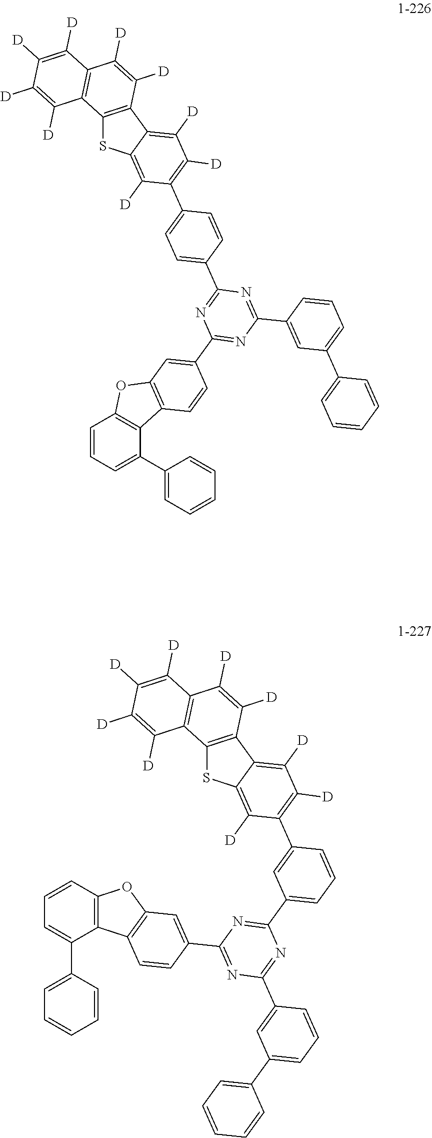 [SMILES: [2H]c1c([2H])c([2H])c2c(c1[2H])c([2H])c([2H])c1c2sc2c([2H])c(-c3ccc(-c4nc(-c5cccc(-c6ccccc6)c5)nc(-c5ccc6c(c5)oc5cccc(-c7ccccc7)c56)n4)cc3)c([2H])c([2H])c21 ; [2H]c1c([2H])c([2H])c2c(c1[2H])c([2H])c([2H])c1c2sc2c([2H])c(-c3cccc(-c4nc(-c5cccc(-c6ccccc6)c5)nc(-c5ccc6c(c5)oc5cccc(-c7ccccc7)c56)n4)c3)c([2H])c([2H])c21]

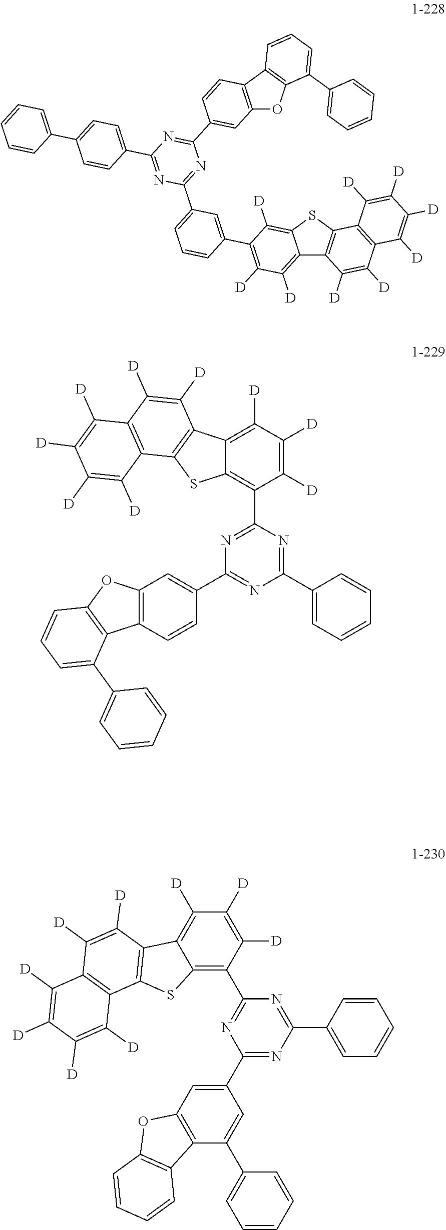 [SMILES: [2H]c1c([2H])c([2H])c2c(c1[2H])c([2H])c([2H])c1c2sc2c([2H])c(-c3cccc(-c4nc(-c5ccc(-c6ccccc6)cc5)nc(-c5ccc6c(c5)oc5c(-c7ccccc7)cccc56)n4)c3)c([2H])c([2H])c21 ; [2H]c1c([2H])c([2H])c2c(sc3c4c([2H])c([2H])c([2H])c([2H])c4c([2H])c([2H])c32)c1-c1nc(-c2ccccc2)nc(-c2ccc3c(c2)oc2cccc(-c4ccccc4)c23)n1 ; [2H]c1c([2H])c([2H])c2c(sc3c4c([2H])c([2H])c([2H])c([2H])c4c([2H])c([2H])c32)c1-c1nc(-c2ccccc2)nc(-c2cc(-c3ccccc3)c3c(c2)oc2ccccc23)n1]

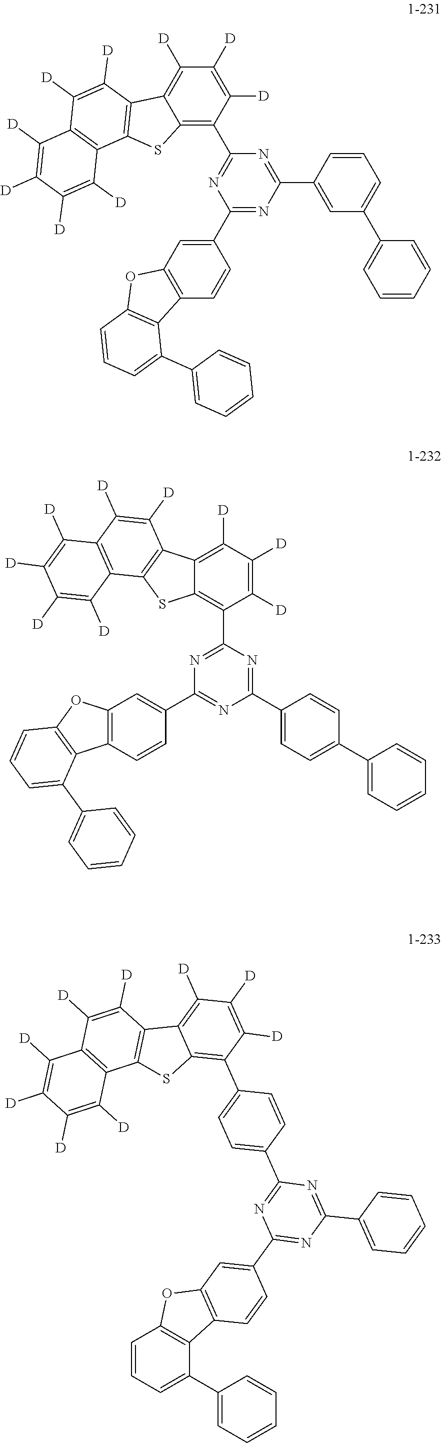 [SMILES: [2H]c1c([2H])c([2H])c2c(sc3c4c([2H])c([2H])c([2H])c([2H])c4c([2H])c([2H])c32)c1-c1nc(-c2cccc(-c3ccccc3)c2)nc(-c2ccc3c(c2)oc2cccc(-c4ccccc4)c23)n1 ; [2H]c1c([2H])c([2H])c2c(sc3c4c([2H])c([2H])c([2H])c([2H])c4c([2H])c([2H])c32)c1-c1nc(-c2ccc(-c3ccccc3)cc2)nc(-c2ccc3c(c2)oc2cccc(-c4ccccc4)c23)n1 ; [2H]c1c([2H])c([2H])c2c(sc3c4c([2H])c([2H])c([2H])c([2H])c4c([2H])c([2H])c32)c1-c1ccc(-c2nc(-c3ccccc3)nc(-c3ccc4c(c3)oc3cccc(-c5ccccc5)c34)n2)cc1]

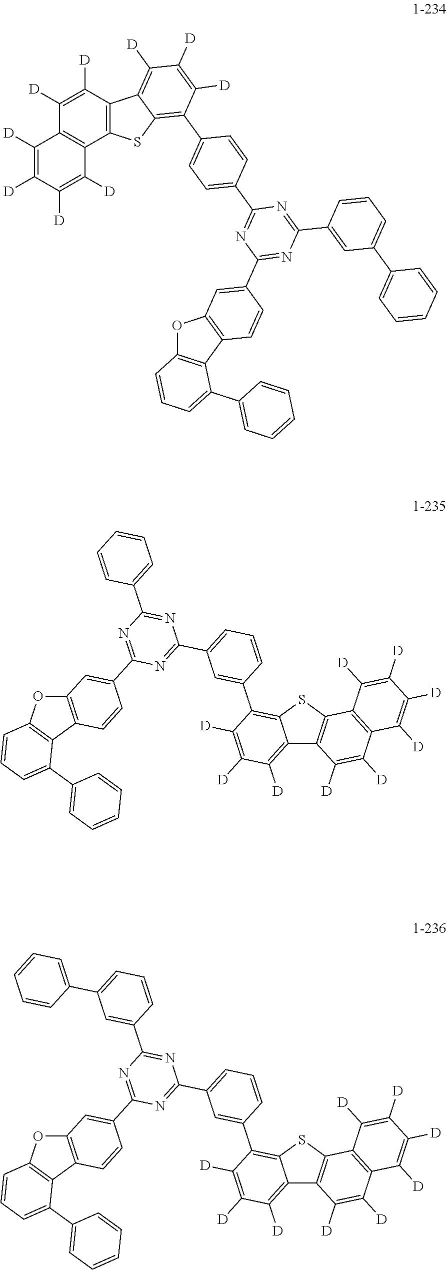 [SMILES: [2H]c1c([2H])c([2H])c2c(sc3c4c([2H])c([2H])c([2H])c([2H])c4c([2H])c([2H])c32)c1-c1ccc(-c2nc(-c3cccc(-c4ccccc4)c3)nc(-c3ccc4c(c3)oc3cccc(-c5ccccc5)c34)n2)cc1 ; [2H]c1c([2H])c([2H])c2c(sc3c4c([2H])c([2H])c([2H])c([2H])c4c([2H])c([2H])c32)c1-c1cccc(-c2nc(-c3ccccc3)nc(-c3ccc4c(c3)oc3cccc(-c5ccccc5)c34)n2)c1 ; [2H]c1c([2H])c([2H])c2c(sc3c4c([2H])c([2H])c([2H])c([2H])c4c([2H])c([2H])c32)c1-c1cccc(-c2nc(-c3cccc(-c4ccccc4)c3)nc(-c3ccc4c(c3)oc3cccc(-c5ccccc5)c34)n2)c1]

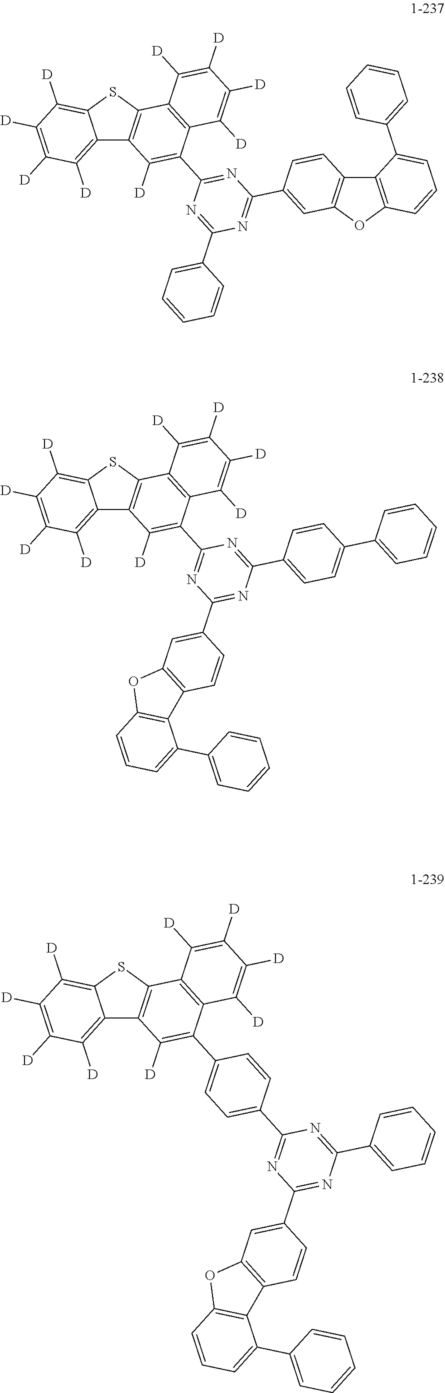 [SMILES: [2H]c1c([2H])c([2H])c2c(sc3c4c([2H])c([2H])c([2H])c([2H])c4c(-c4nc(-c5ccccc5)nc(-c5ccc6c(c5)oc5cccc(-c7ccccc7)c56)n4)c([2H])c32)c1[2H] ; [2H]c1c([2H])c([2H])c2c(sc3c4c([2H])c([2H])c([2H])c([2H])c4c(-c4nc(-c5ccc(-c6ccccc6)cc5)nc(-c5ccc6c(c5)oc5cccc(-c7ccccc7)c56)n4)c([2H])c32)c1[2H] ; [2H]c1c([2H])c([2H])c2c(sc3c4c([2H])c([2H])c([2H])c([2H])c4c(-c4ccc(-c5nc(-c6ccccc6)nc(-c6ccc7c(c6)oc6cccc(-c8ccccc8)c67)n5)cc4)c([2H])c32)c1[2H]]

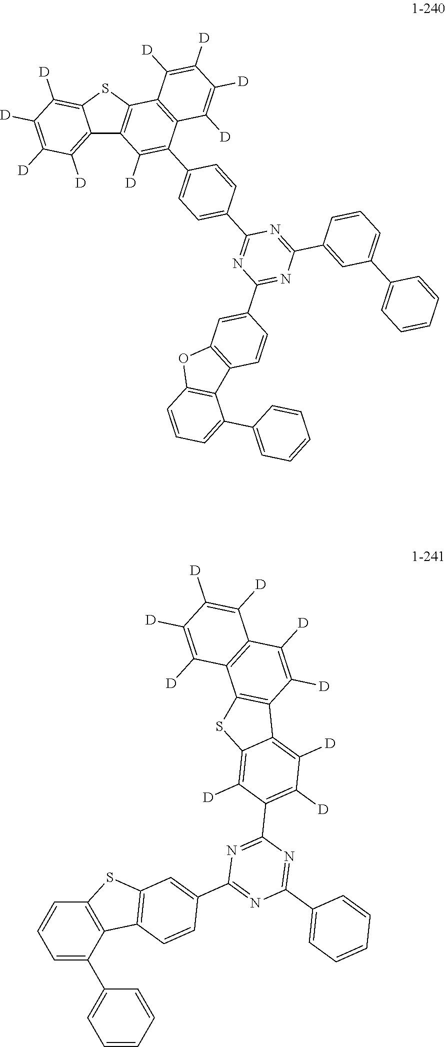 [SMILES: [2H]c1c([2H])c([2H])c2c(sc3c4c([2H])c([2H])c([2H])c([2H])c4c(-c4ccc(-c5nc(-c6cccc(-c7ccccc7)c6)nc(-c6ccc7c(c6)oc6cccc(-c8ccccc8)c67)n5)cc4)c([2H])c32)c1[2H] ; [2H]c1c([2H])c([2H])c2c(c1[2H])c([2H])c([2H])c1c2sc2c([2H])c(-c3nc(-c4ccccc4)nc(-c4ccc5c(c4)sc4cccc(-c6ccccc6)c45)n3)c([2H])c([2H])c21]

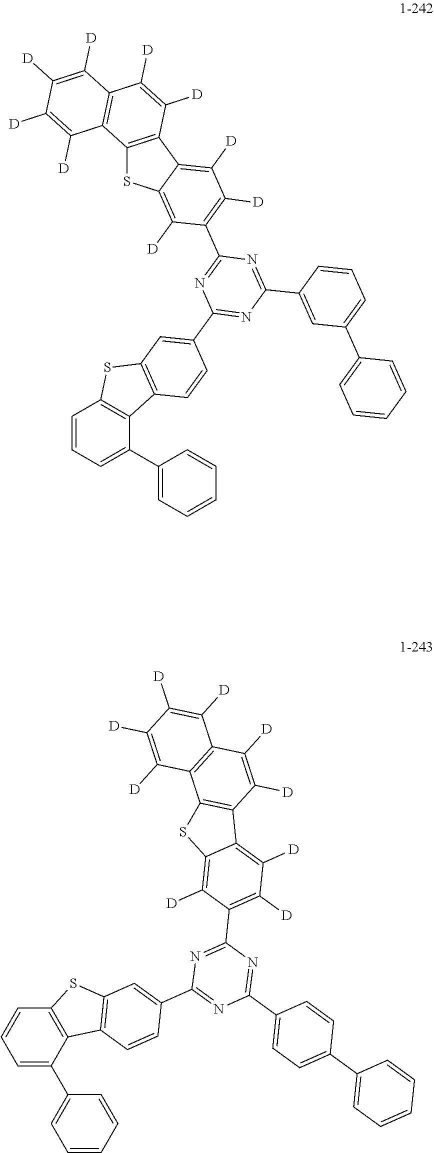 [SMILES: [2H]c1c([2H])c([2H])c2c(c1[2H])c([2H])c([2H])c1c2sc2c([2H])c(-c3nc(-c4cccc(-c5ccccc5)c4)nc(-c4ccc5c(c4)sc4cccc(-c6ccccc6)c45)n3)c([2H])c([2H])c21 ; [2H]c1c([2H])c([2H])c2c(c1[2H])c([2H])c([2H])c1c2sc2c([2H])c(-c3nc(-c4ccc(-c5ccccc5)cc4)nc(-c4ccc5c(c4)sc4cccc(-c6ccccc6)c45)n3)c([2H])c([2H])c21]

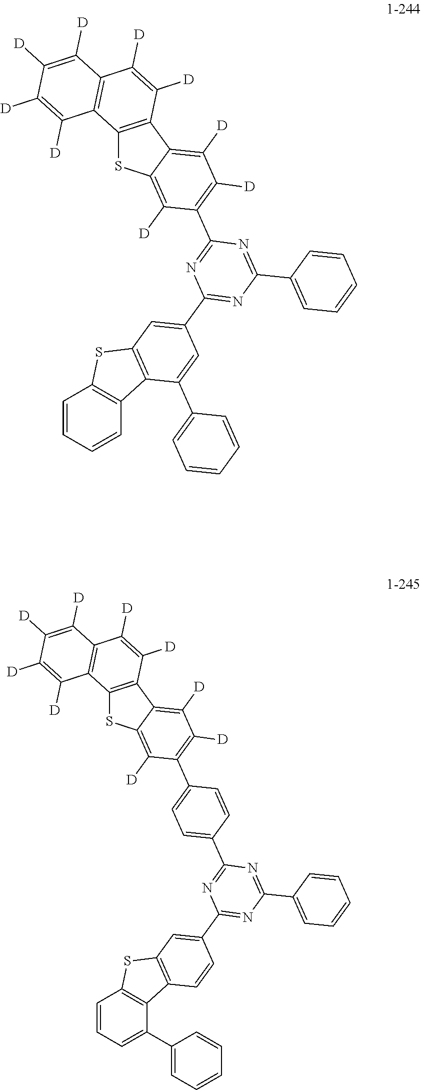 [SMILES: [2H]c1c([2H])c([2H])c2c(c1[2H])c([2H])c([2H])c1c2sc2c([2H])c(-c3nc(-c4ccccc4)nc(-c4cc(-c5ccccc5)c5c(c4)sc4ccccc45)n3)c([2H])c([2H])c21 ; [2H]c1c([2H])c([2H])c2c(c1[2H])c([2H])c([2H])c1c2sc2c([2H])c(-c3ccc(-c4nc(-c5ccccc5)nc(-c5ccc6c(c5)sc5cccc(-c7ccccc7)c56)n4)cc3)c([2H])c([2H])c21]

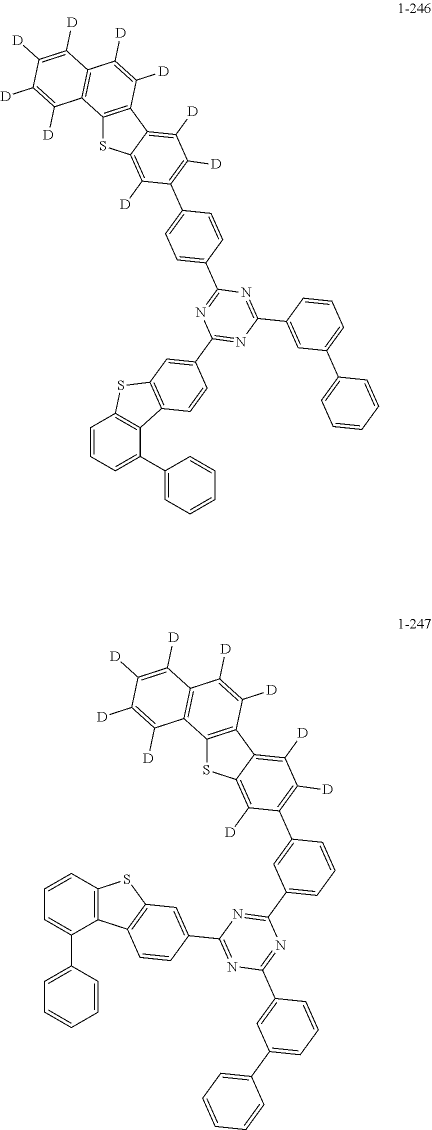 [SMILES: [2H]c1c([2H])c([2H])c2c(c1[2H])c([2H])c([2H])c1c2sc2c([2H])c(-c3ccc(-c4nc(-c5cccc(-c6ccccc6)c5)nc(-c5ccc6c(c5)sc5cccc(-c7ccccc7)c56)n4)cc3)c([2H])c([2H])c21 ; [2H]c1c([2H])c([2H])c2c(c1[2H])c([2H])c([2H])c1c2sc2c([2H])c(-c3cccc(-c4nc(-c5cccc(-c6ccccc6)c5)nc(-c5ccc6c(c5)sc5cccc(-c7ccccc7)c56)n4)c3)c([2H])c([2H])c21]

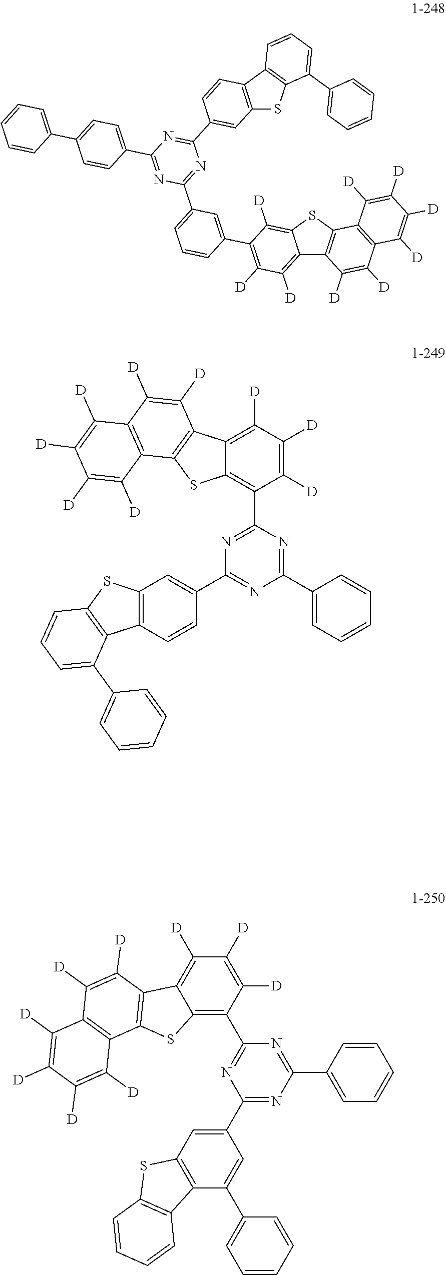 [SMILES: [2H]c1c([2H])c([2H])c2c(c1[2H])c([2H])c([2H])c1c2sc2c([2H])c(-c3cccc(-c4nc(-c5ccc(-c6ccccc6)cc5)nc(-c5ccc6c(c5)sc5c(-c7ccccc7)cccc56)n4)c3)c([2H])c([2H])c21 ; [2H]c1c([2H])c([2H])c2c(sc3c4c([2H])c([2H])c([2H])c([2H])c4c([2H])c([2H])c32)c1-c1nc(-c2ccccc2)nc(-c2ccc3c(c2)sc2cccc(-c4ccccc4)c23)n1 ; [2H]c1c([2H])c([2H])c2c(sc3c4c([2H])c([2H])c([2H])c([2H])c4c([2H])c([2H])c32)c1-c1nc(-c2ccccc2)nc(-c2cc(-c3ccccc3)c3c(c2)sc2ccccc23)n1]

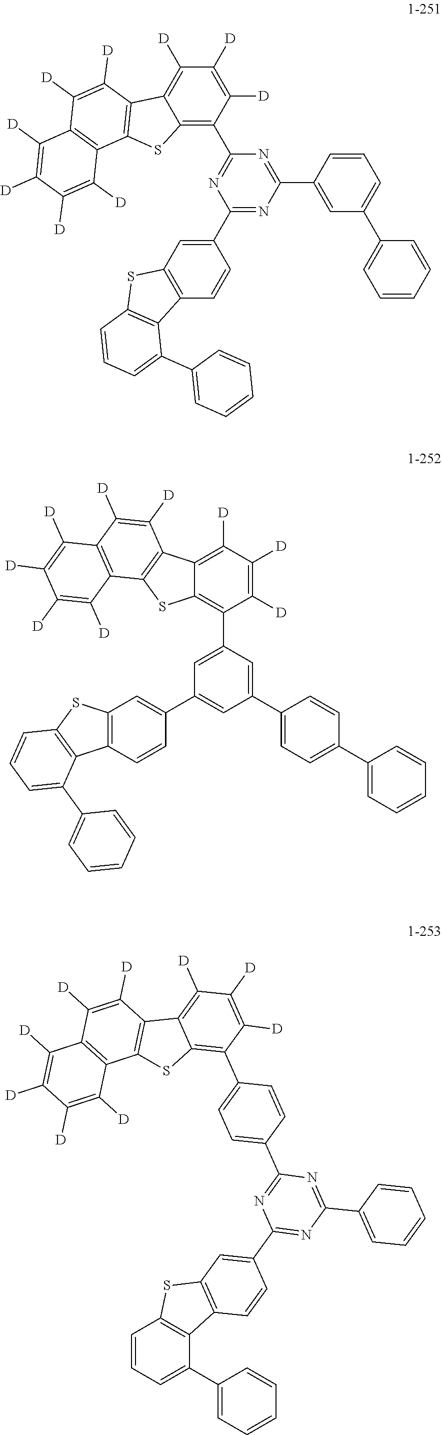 [SMILES: [2H]c1c([2H])c([2H])c2c(sc3c4c([2H])c([2H])c([2H])c([2H])c4c([2H])c([2H])c32)c1-c1nc(-c2cccc(-c3ccccc3)c2)nc(-c2ccc3c(c2)sc2cccc(-c4ccccc4)c23)n1 ; [2H]c1c([2H])c([2H])c2c(sc3c4c([2H])c([2H])c([2H])c([2H])c4c([2H])c([2H])c32)c1-c1cc(-c2ccc(-c3ccccc3)cc2)cc(-c2ccc3c(c2)sc2cccc(-c4ccccc4)c23)c1 ; [2H]c1c([2H])c([2H])c2c(sc3c4c([2H])c([2H])c([2H])c([2H])c4c([2H])c([2H])c32)c1-c1ccc(-c2nc(-c3ccccc3)nc(-c3ccc4c(c3)sc3cccc(-c5ccccc5)c34)n2)cc1]

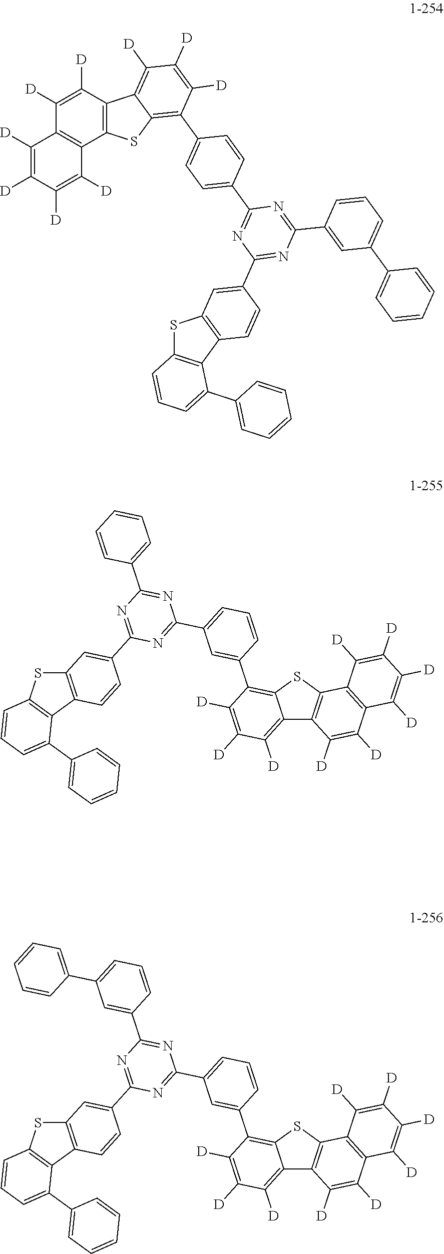 [SMILES: [2H]c1c([2H])c([2H])c2c(sc3c4c([2H])c([2H])c([2H])c([2H])c4c([2H])c([2H])c32)c1-c1ccc(-c2nc(-c3cccc(-c4ccccc4)c3)nc(-c3ccc4c(c3)sc3cccc(-c5ccccc5)c34)n2)cc1 ; [2H]c1c([2H])c([2H])c2c(sc3c4c([2H])c([2H])c([2H])c([2H])c4c([2H])c([2H])c32)c1-c1cccc(-c2nc(-c3ccccc3)nc(-c3ccc4c(c3)sc3cccc(-c5ccccc5)c34)n2)c1 ; [2H]c1c([2H])c([2H])c2c(sc3c4c([2H])c([2H])c([2H])c([2H])c4c([2H])c([2H])c32)c1-c1cccc(-c2nc(-c3cccc(-c4ccccc4)c3)nc(-c3ccc4c(c3)sc3cccc(-c5ccccc5)c34)n2)c1]

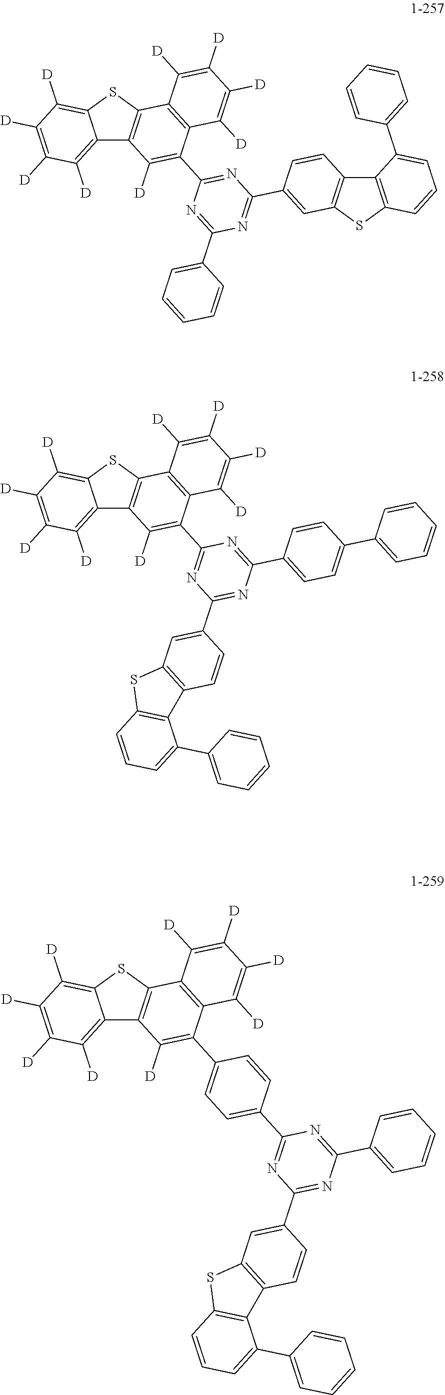 [SMILES: [2H]c1c([2H])c([2H])c2c(sc3c4c([2H])c([2H])c([2H])c([2H])c4c(-c4nc(-c5ccccc5)nc(-c5ccc6c(c5)sc5cccc(-c7ccccc7)c56)n4)c([2H])c32)c1[2H] ; [2H]c1c([2H])c([2H])c2c(sc3c4c([2H])c([2H])c([2H])c([2H])c4c(-c4nc(-c5ccc(-c6ccccc6)cc5)nc(-c5ccc6c(c5)sc5cccc(-c7ccccc7)c56)n4)c([2H])c32)c1[2H] ; [2H]c1c([2H])c([2H])c2c(sc3c4c([2H])c([2H])c([2H])c([2H])c4c(-c4ccc(-c5nc(-c6ccccc6)nc(-c6ccc7c(c6)sc6cccc(-c8ccccc8)c67)n5)cc4)c([2H])c32)c1[2H]]

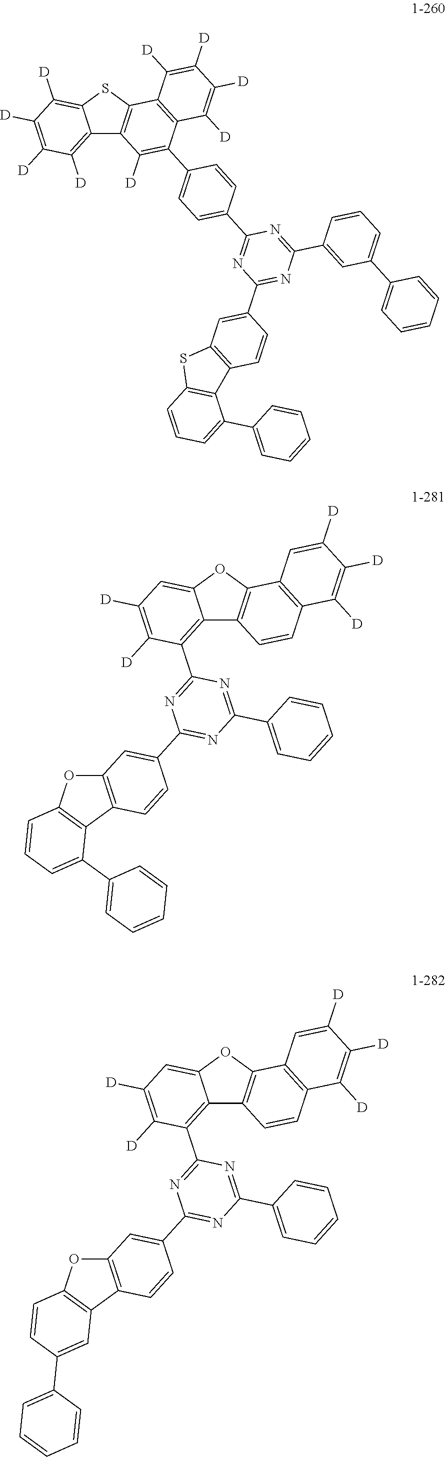 [SMILES: [2H]c1c([2H])c([2H])c2c(sc3c4c([2H])c([2H])c([2H])c([2H])c4c(-c4ccc(-c5nc(-c6cccc(-c7ccccc7)c6)nc(-c6ccc7c(c6)sc6cccc(-c8ccccc8)c67)n5)cc4)c([2H])c32)c1[2H] ; [2H]c1cc2c(ccc3c2oc2cc([2H])c([2H])c(-c4nc(-c5ccccc5)nc(-c5ccc6c(c5)oc5cccc(-c7ccccc7)c56)n4)c23)c([2H])c1[2H] ; [2H]c1cc2c(ccc3c2oc2cc([2H])c([2H])c(-c4nc(-c5ccccc5)nc(-c5ccc6c(c5)oc5ccc(-c7ccccc7)cc56)n4)c23)c([2H])c1[2H]]

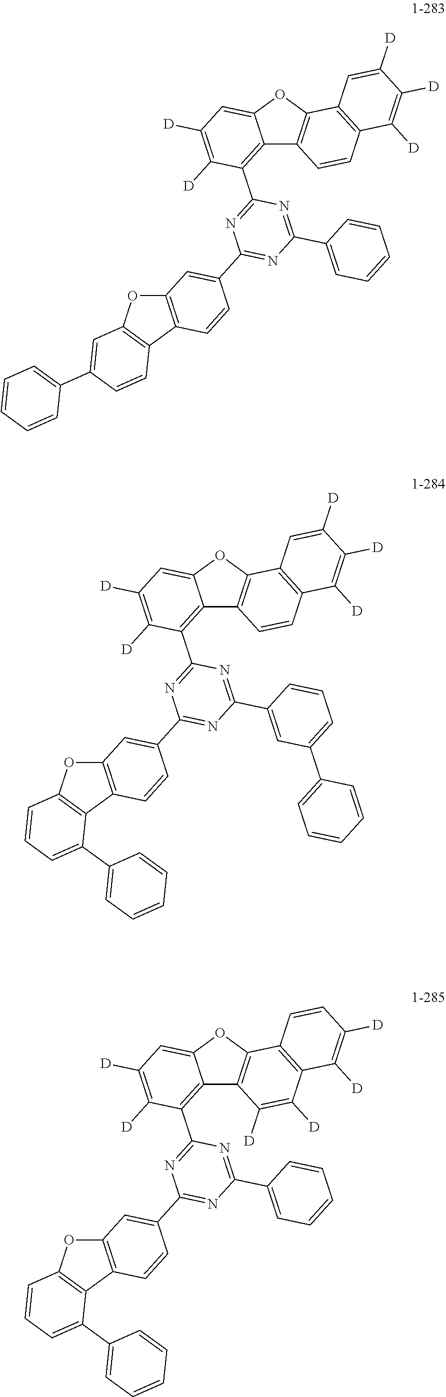 [SMILES: [2H]c1cc2c(ccc3c2oc2cc([2H])c([2H])c(-c4nc(-c5ccccc5)nc(-c5ccc6c(c5)oc5cc(-c7ccccc7)ccc56)n4)c23)c([2H])c1[2H] ; [2H]c1cc2c(ccc3c2oc2cc([2H])c([2H])c(-c4nc(-c5cccc(-c6ccccc6)c5)nc(-c5ccc6c(c5)oc5cccc(-c7ccccc7)c56)n4)c23)c([2H])c1[2H] ; [2H]c1cc2oc3c4ccc([2H])c([2H])c4c([2H])c([2H])c3c2c(-c2nc(-c3ccccc3)nc(-c3ccc4c(c3)oc3cccc(-c5ccccc5)c34)n2)c1[2H]]

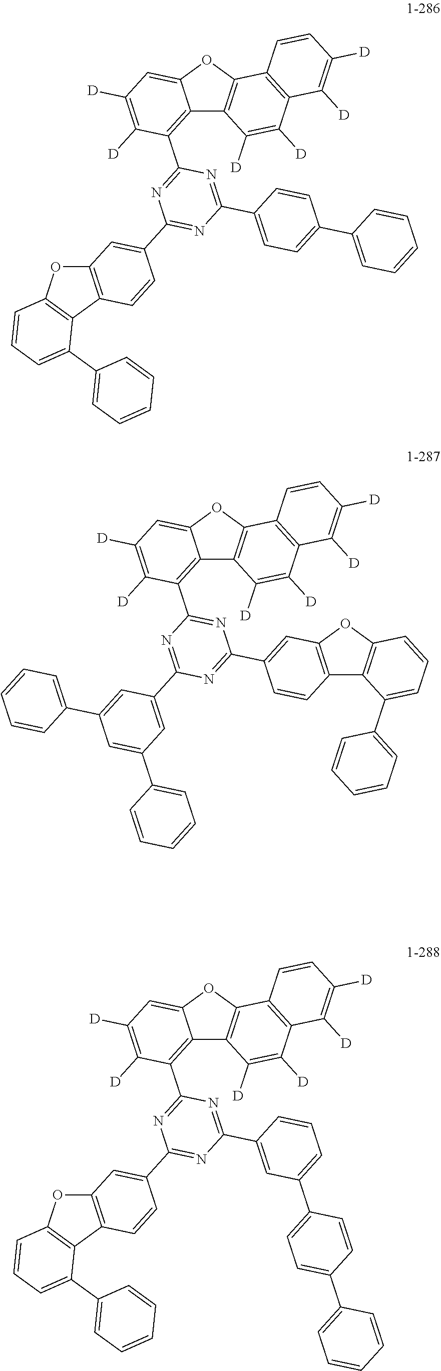 [SMILES: [2H]c1cc2oc3c4ccc([2H])c([2H])c4c([2H])c([2H])c3c2c(-c2nc(-c3ccc(-c4ccccc4)cc3)nc(-c3ccc4c(c3)oc3cccc(-c5ccccc5)c34)n2)c1[2H] ; [2H]c1cc2oc3c4ccc([2H])c([2H])c4c([2H])c([2H])c3c2c(-c2nc(-c3cc(-c4ccccc4)cc(-c4ccccc4)c3)nc(-c3ccc4c(c3)oc3cccc(-c5ccccc5)c34)n2)c1[2H] ; [2H]c1cc2oc3c4ccc([2H])c([2H])c4c([2H])c([2H])c3c2c(-c2nc(-c3cccc(-c4ccc(-c5ccccc5)cc4)c3)nc(-c3ccc4c(c3)oc3cccc(-c5ccccc5)c34)n2)c1[2H]]

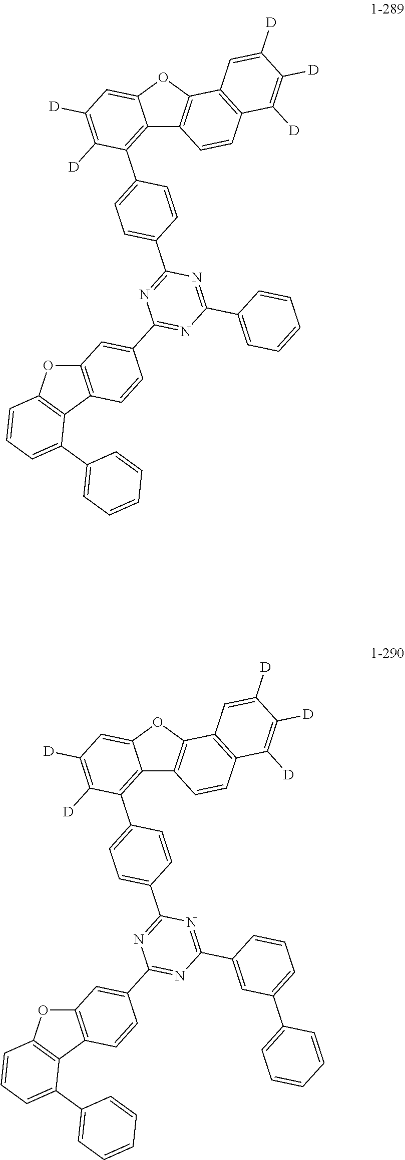 [SMILES: [2H]c1cc2c(ccc3c2oc2cc([2H])c([2H])c(-c4ccc(-c5nc(-c6ccccc6)nc(-c6ccc7c(c6)oc6cccc(-c8ccccc8)c67)n5)cc4)c23)c([2H])c1[2H] ; [2H]c1cc2c(ccc3c2oc2cc([2H])c([2H])c(-c4ccc(-c5nc(-c6cccc(-c7ccccc7)c6)nc(-c6ccc7c(c6)oc6cccc(-c8ccccc8)c67)n5)cc4)c23)c([2H])c1[2H]]

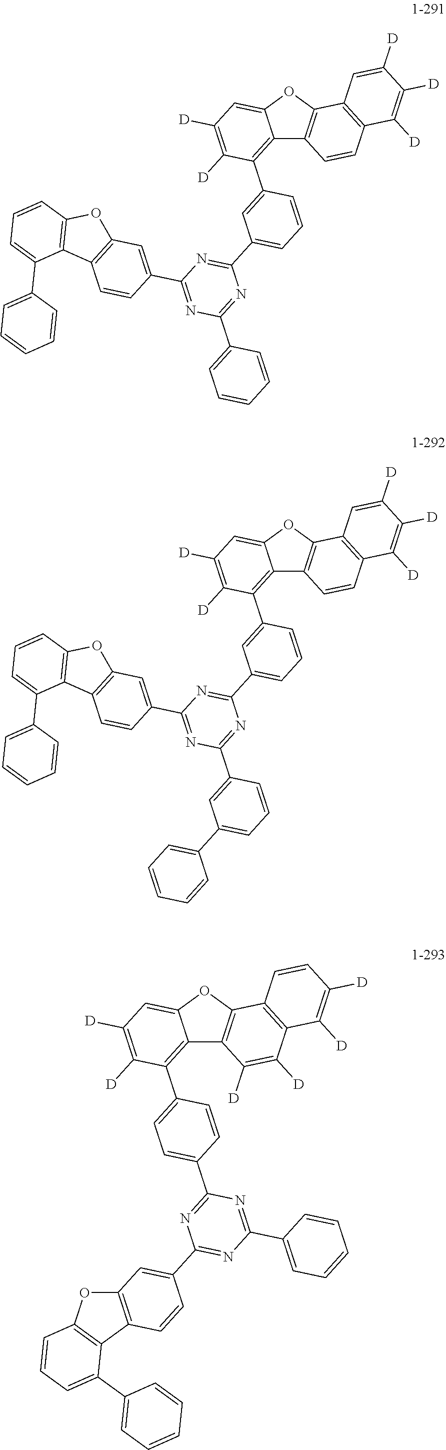 [SMILES: [2H]c1cc2c(ccc3c2oc2cc([2H])c([2H])c(-c4cccc(-c5nc(-c6ccccc6)nc(-c6ccc7c(c6)oc6cccc(-c8ccccc8)c67)n5)c4)c23)c([2H])c1[2H] ; [2H]c1cc2c(ccc3c2oc2cc([2H])c([2H])c(-c4cccc(-c5nc(-c6cccc(-c7ccccc7)c6)nc(-c6ccc7c(c6)oc6cccc(-c8ccccc8)c67)n5)c4)c23)c([2H])c1[2H] ; [2H]c1cc2oc3c4ccc([2H])c([2H])c4c([2H])c([2H])c3c2c(-c2ccc(-c3nc(-c4ccccc4)nc(-c4ccc5c(c4)oc4cccc(-c6ccccc6)c45)n3)cc2)c1[2H]]

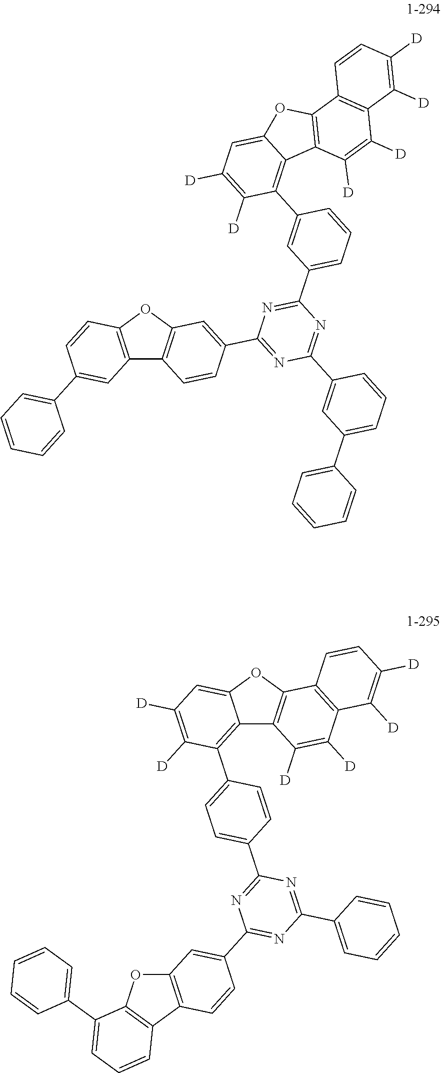 [SMILES: [2H]c1cc2oc3c4ccc([2H])c([2H])c4c([2H])c([2H])c3c2c(-c2cccc(-c3nc(-c4cccc(-c5ccccc5)c4)nc(-c4ccc5c(c4)oc4ccc(-c6ccccc6)cc45)n3)c2)c1[2H] ; [2H]c1cc2oc3c4ccc([2H])c([2H])c4c([2H])c([2H])c3c2c(-c2ccc(-c3nc(-c4ccccc4)nc(-c4ccc5c(c4)oc4c(-c6ccccc6)cccc45)n3)cc2)c1[2H]]

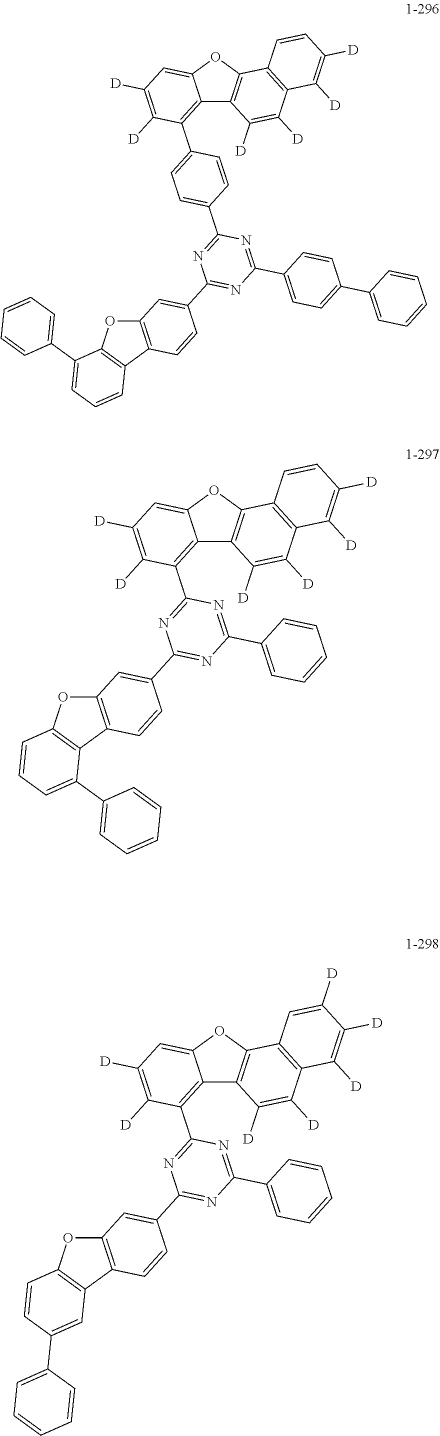 [SMILES: [2H]c1cc2oc3c4ccc([2H])c([2H])c4c([2H])c([2H])c3c2c(-c2ccc(-c3nc(-c4ccc(-c5ccccc5)cc4)nc(-c4ccc5c(c4)oc4c(-c6ccccc6)cccc45)n3)cc2)c1[2H] ; [2H]c1cc2oc3c4ccc([2H])c([2H])c4c([2H])c([2H])c3c2c(-c2nc(-c3ccccc3)nc(-c3ccc4c(c3)oc3cccc(-c5ccccc5)c34)n2)c1[2H] ; [2H]c1cc2c(c([2H])c1[2H])c([2H])c([2H])c1c2oc2cc([2H])c([2H])c(-c3nc(-c4ccccc4)nc(-c4ccc5c(c4)oc4ccc(-c6ccccc6)cc45)n3)c21]

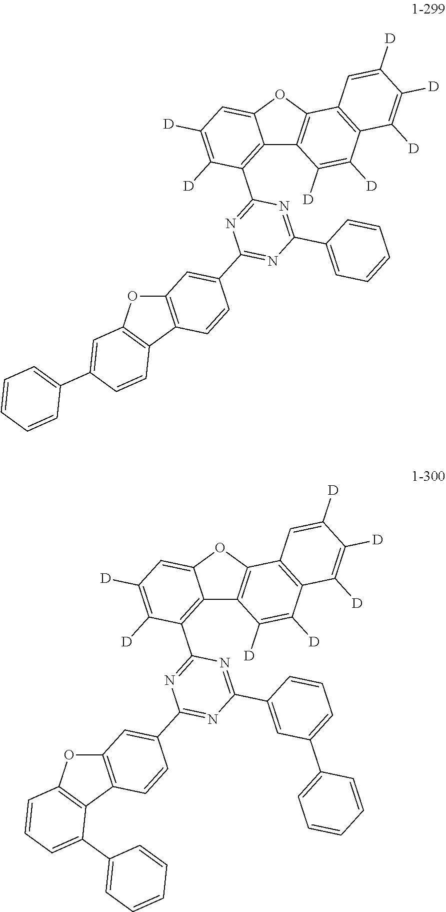 [SMILES: [2H]c1cc2c(c([2H])c1[2H])c([2H])c([2H])c1c2oc2cc([2H])c([2H])c(-c3nc(-c4ccccc4)nc(-c4ccc5c(c4)oc4cc(-c6ccccc6)ccc45)n3)c21 ; [2H]c1cc2c(c([2H])c1[2H])c([2H])c([2H])c1c2oc2cc([2H])c([2H])c(-c3nc(-c4cccc(-c5ccccc5)c4)nc(-c4ccc5c(c4)oc4cccc(-c6ccccc6)c45)n3)c21]

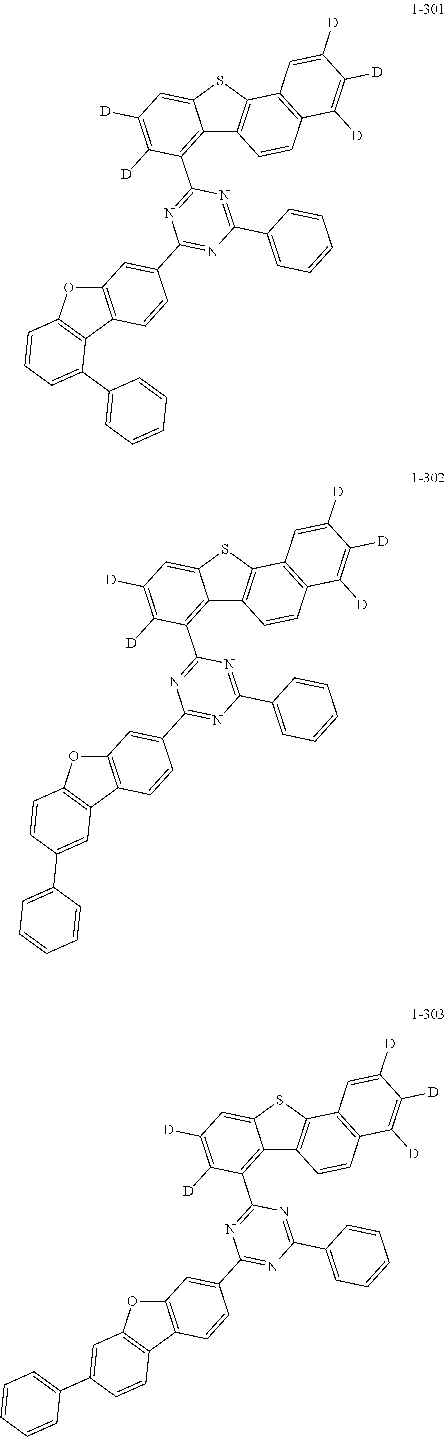 [SMILES: [2H]c1cc2c(ccc3c2sc2cc([2H])c([2H])c(-c4nc(-c5ccccc5)nc(-c5ccc6c(c5)oc5cccc(-c7ccccc7)c56)n4)c23)c([2H])c1[2H] ; [2H]c1cc2c(ccc3c2sc2cc([2H])c([2H])c(-c4nc(-c5ccccc5)nc(-c5ccc6c(c5)oc5ccc(-c7ccccc7)cc56)n4)c23)c([2H])c1[2H] ; [2H]c1cc2c(ccc3c2sc2cc([2H])c([2H])c(-c4nc(-c5ccccc5)nc(-c5ccc6c(c5)oc5cc(-c7ccccc7)ccc56)n4)c23)c([2H])c1[2H]]

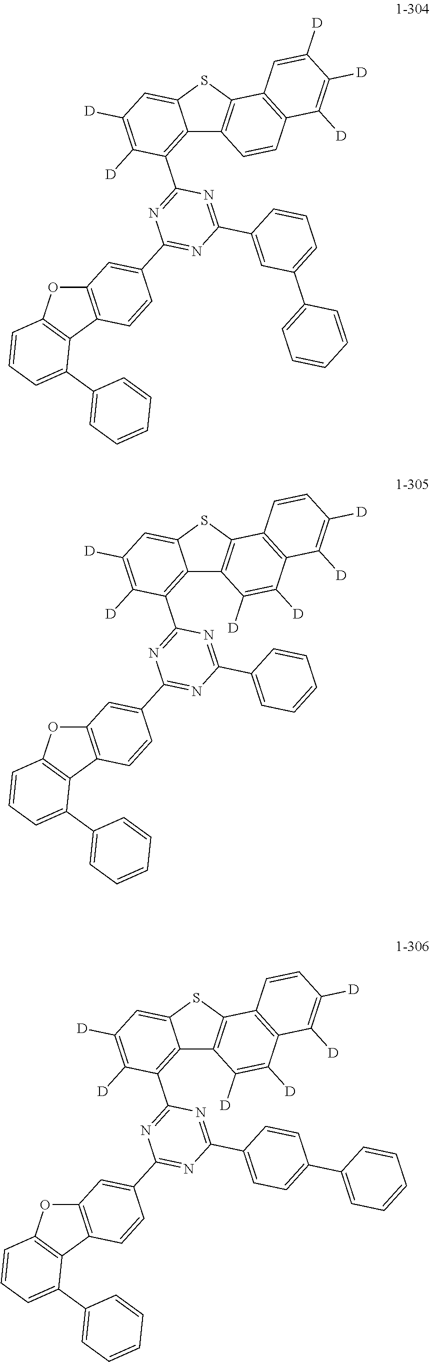 [SMILES: [2H]c1cc2c(ccc3c2sc2cc([2H])c([2H])c(-c4nc(-c5cccc(-c6ccccc6)c5)nc(-c5ccc6c(c5)oc5cccc(-c7ccccc7)c56)n4)c23)c([2H])c1[2H] ; [2H]c1cc2sc3c4ccc([2H])c([2H])c4c([2H])c([2H])c3c2c(-c2nc(-c3ccccc3)nc(-c3ccc4c(c3)oc3cccc(-c5ccccc5)c34)n2)c1[2H] ; [2H]c1cc2sc3c4ccc([2H])c([2H])c4c([2H])c([2H])c3c2c(-c2nc(-c3ccc(-c4ccccc4)cc3)nc(-c3ccc4c(c3)oc3cccc(-c5ccccc5)c34)n2)c1[2H]]

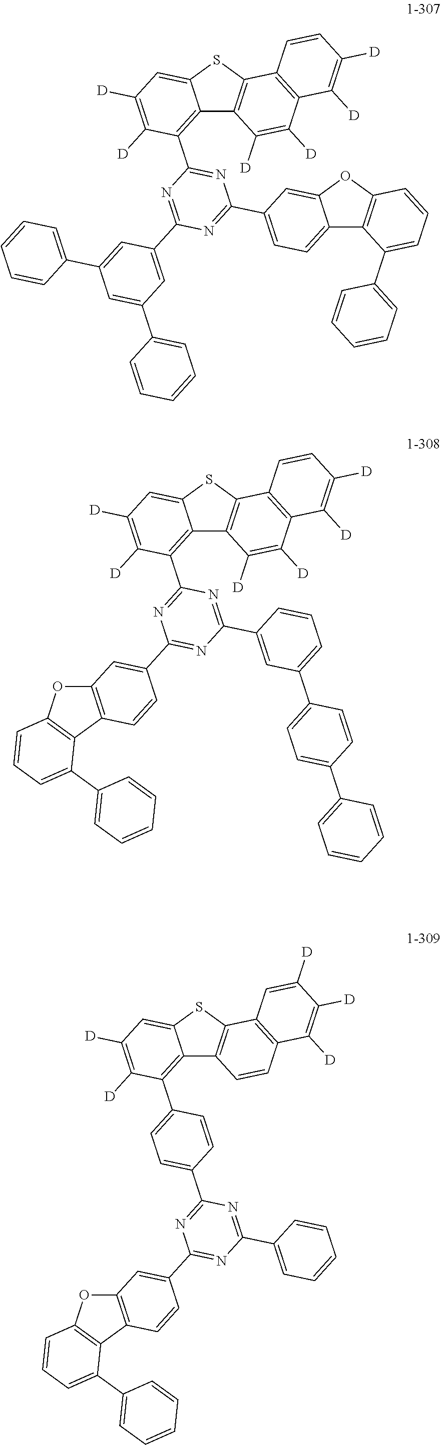 [SMILES: [2H]c1cc2sc3c4ccc([2H])c([2H])c4c([2H])c([2H])c3c2c(-c2nc(-c3cc(-c4ccccc4)cc(-c4ccccc4)c3)nc(-c3ccc4c(c3)oc3cccc(-c5ccccc5)c34)n2)c1[2H] ; [2H]c1cc2sc3c4ccc([2H])c([2H])c4c([2H])c([2H])c3c2c(-c2nc(-c3cccc(-c4ccc(-c5ccccc5)cc4)c3)nc(-c3ccc4c(c3)oc3cccc(-c5ccccc5)c34)n2)c1[2H] ; [2H]c1cc2c(ccc3c2sc2cc([2H])c([2H])c(-c4ccc(-c5nc(-c6ccccc6)nc(-c6ccc7c(c6)oc6cccc(-c8ccccc8)c67)n5)cc4)c23)c([2H])c1[2H]]

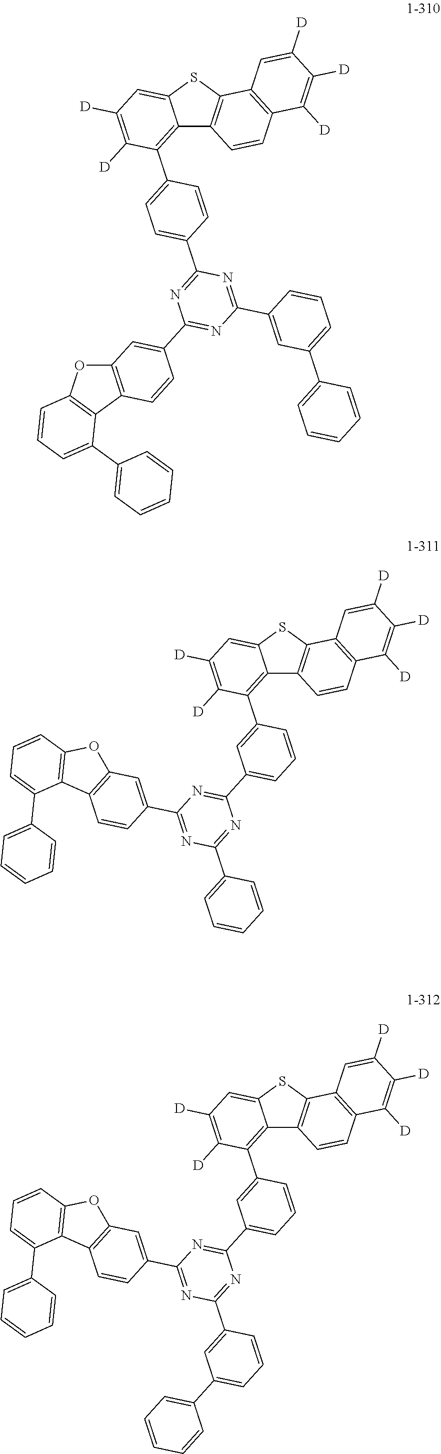 [SMILES: [2H]c1cc2c(ccc3c2sc2cc([2H])c([2H])c(-c4ccc(-c5nc(-c6cccc(-c7ccccc7)c6)nc(-c6ccc7c(c6)oc6cccc(-c8ccccc8)c67)n5)cc4)c23)c([2H])c1[2H] ; [2H]c1cc2c(ccc3c2sc2cc([2H])c([2H])c(-c4cccc(-c5nc(-c6ccccc6)nc(-c6ccc7c(c6)oc6cccc(-c8ccccc8)c67)n5)c4)c23)c([2H])c1[2H] ; [2H]c1cc2c(ccc3c2sc2cc([2H])c([2H])c(-c4cccc(-c5nc(-c6cccc(-c7ccccc7)c6)nc(-c6ccc7c(c6)oc6cccc(-c8ccccc8)c67)n5)c4)c23)c([2H])c1[2H]]

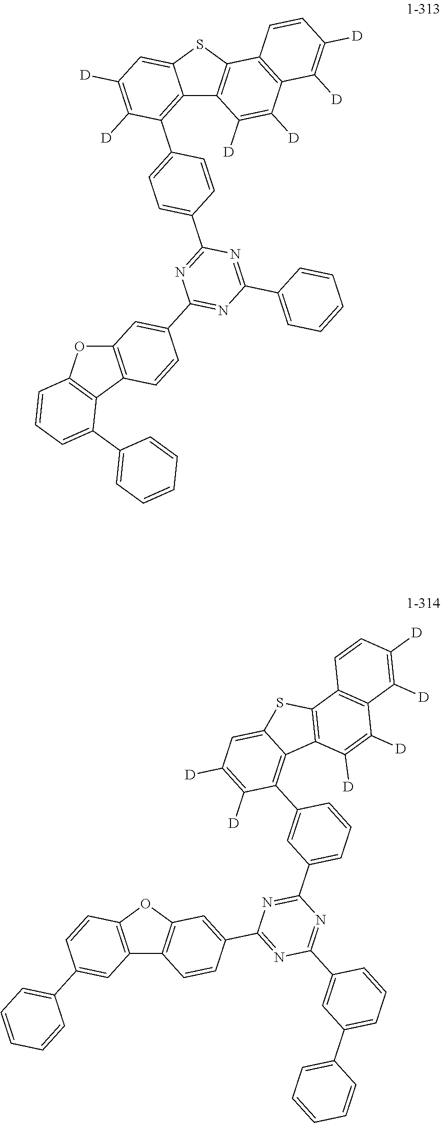 [SMILES: [2H]c1cc2sc3c4ccc([2H])c([2H])c4c([2H])c([2H])c3c2c(-c2ccc(-c3nc(-c4ccccc4)nc(-c4ccc5c(c4)oc4cccc(-c6ccccc6)c45)n3)cc2)c1[2H] ; [2H]c1cc2sc3c4ccc([2H])c([2H])c4c([2H])c([2H])c3c2c(-c2cccc(-c3nc(-c4cccc(-c5ccccc5)c4)nc(-c4ccc5c(c4)oc4ccc(-c6ccccc6)cc45)n3)c2)c1[2H]]

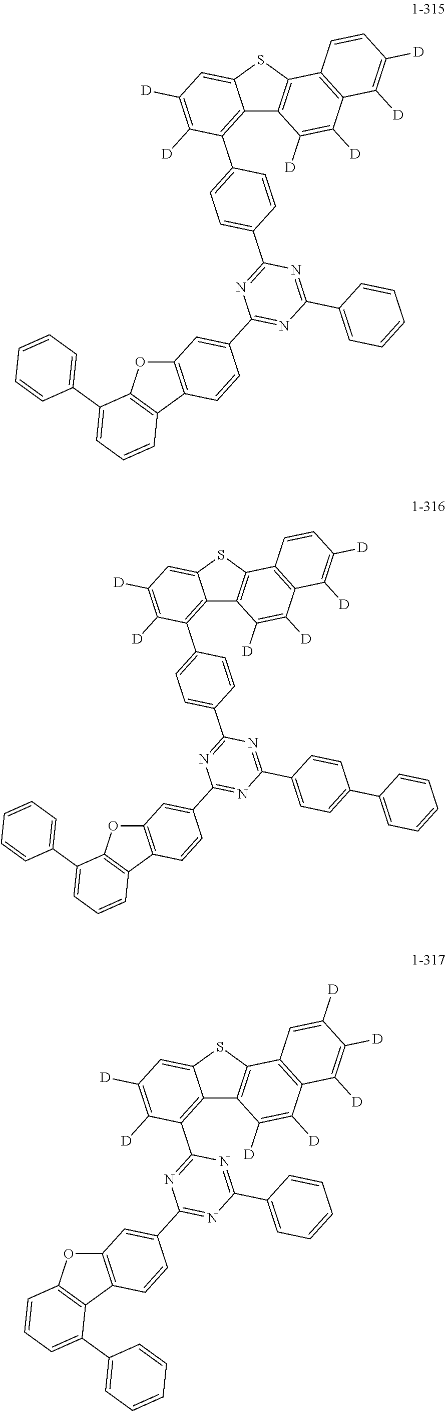 [SMILES: [2H]c1cc2sc3c4ccc([2H])c([2H])c4c([2H])c([2H])c3c2c(-c2ccc(-c3nc(-c4ccccc4)nc(-c4ccc5c(c4)oc4c(-c6ccccc6)cccc45)n3)cc2)c1[2H] ; [2H]c1cc2sc3c4ccc([2H])c([2H])c4c([2H])c([2H])c3c2c(-c2ccc(-c3nc(-c4ccc(-c5ccccc5)cc4)nc(-c4ccc5c(c4)oc4c(-c6ccccc6)cccc45)n3)cc2)c1[2H] ; [2H]c1cc2c(c([2H])c1[2H])c([2H])c([2H])c1c2sc2cc([2H])c([2H])c(-c3nc(-c4ccccc4)nc(-c4ccc5c(c4)oc4cccc(-c6ccccc6)c45)n3)c21]

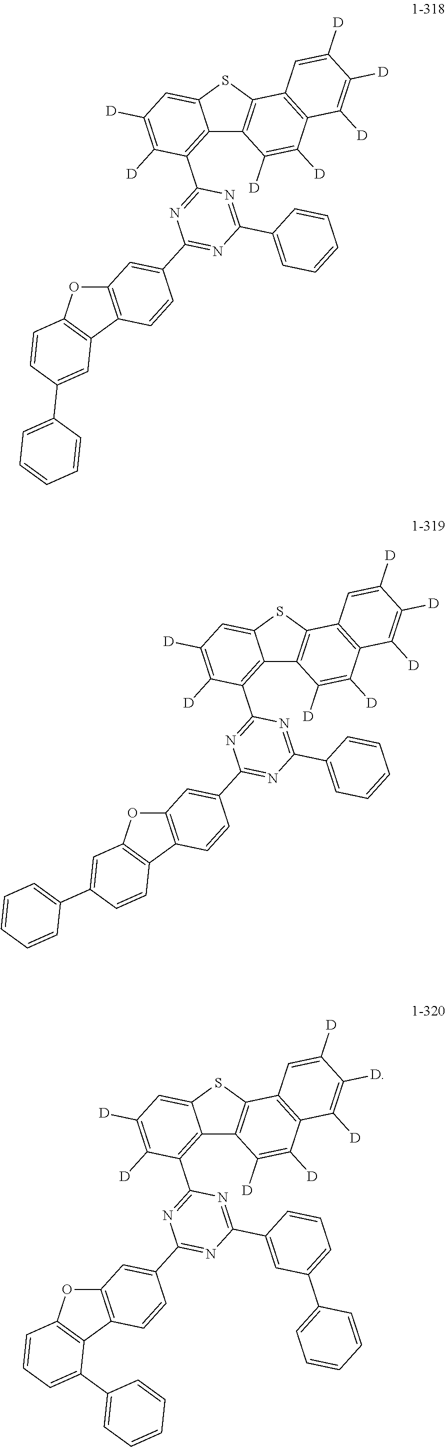

10. An organic light emitting device, comprising:
a first electrode; a second electrode; and one or more organic material layers disposed between the first electrode and the second electrode,
wherein one or more of the organic material layers comprise the heterocyclic compound claim 1.

11. The organic light emitting device of claim 10, wherein the organic layer comprising the heterocyclic compound further comprises a compound of Formula 2 below: 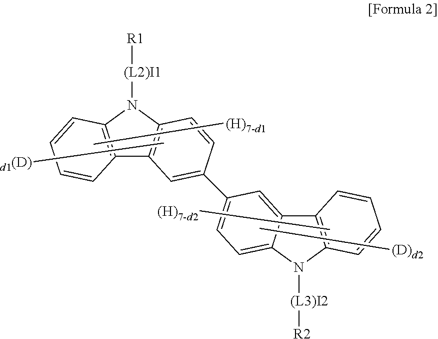
In Formula 2
L2 and L3 are each independently a direct bond; or a substituted or unsubstituted arylene group having 6 to 60 carbon atoms,
each of I1 and I2 is an integer of 1 to 3, and when it is 2 or higher, each substituent in the parentheses is the same or different,
R1 and R2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group 6 to 60 carbon atoms; or a substituted or unsubstituted heteroaryl group having 2 to 60 carbon atoms,
H is hydrogen, and D is deuterium, and
d1 and d2 are each independently an integer of 0 to 7.

12. The organic light emitting device of claim 11, wherein the deuterium content in Formula 2 is 0%, or 10% to 100%.

13. The organic light emitting device of claim 11, wherein Formula 2 is represented by any one of the following compounds:

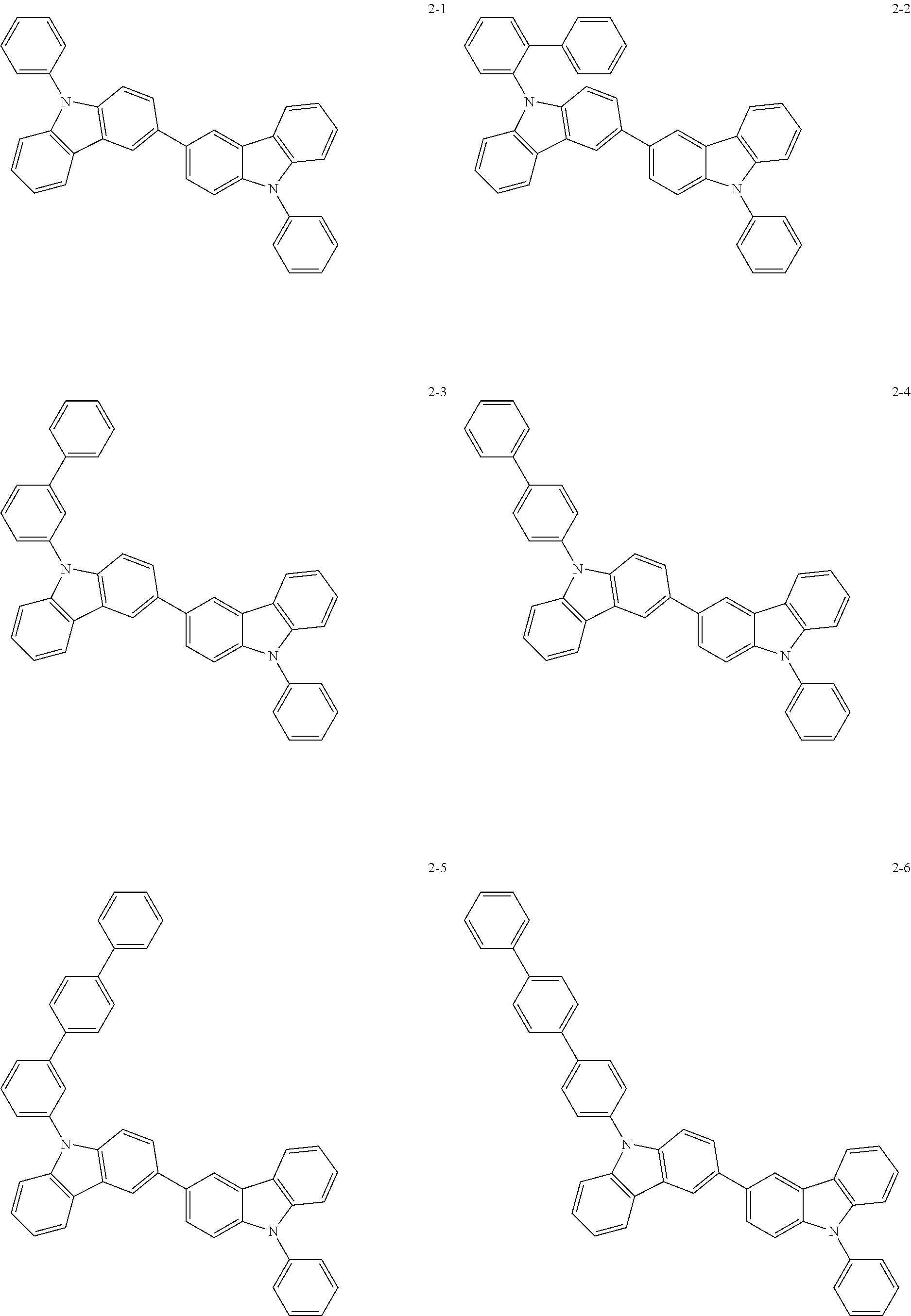 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

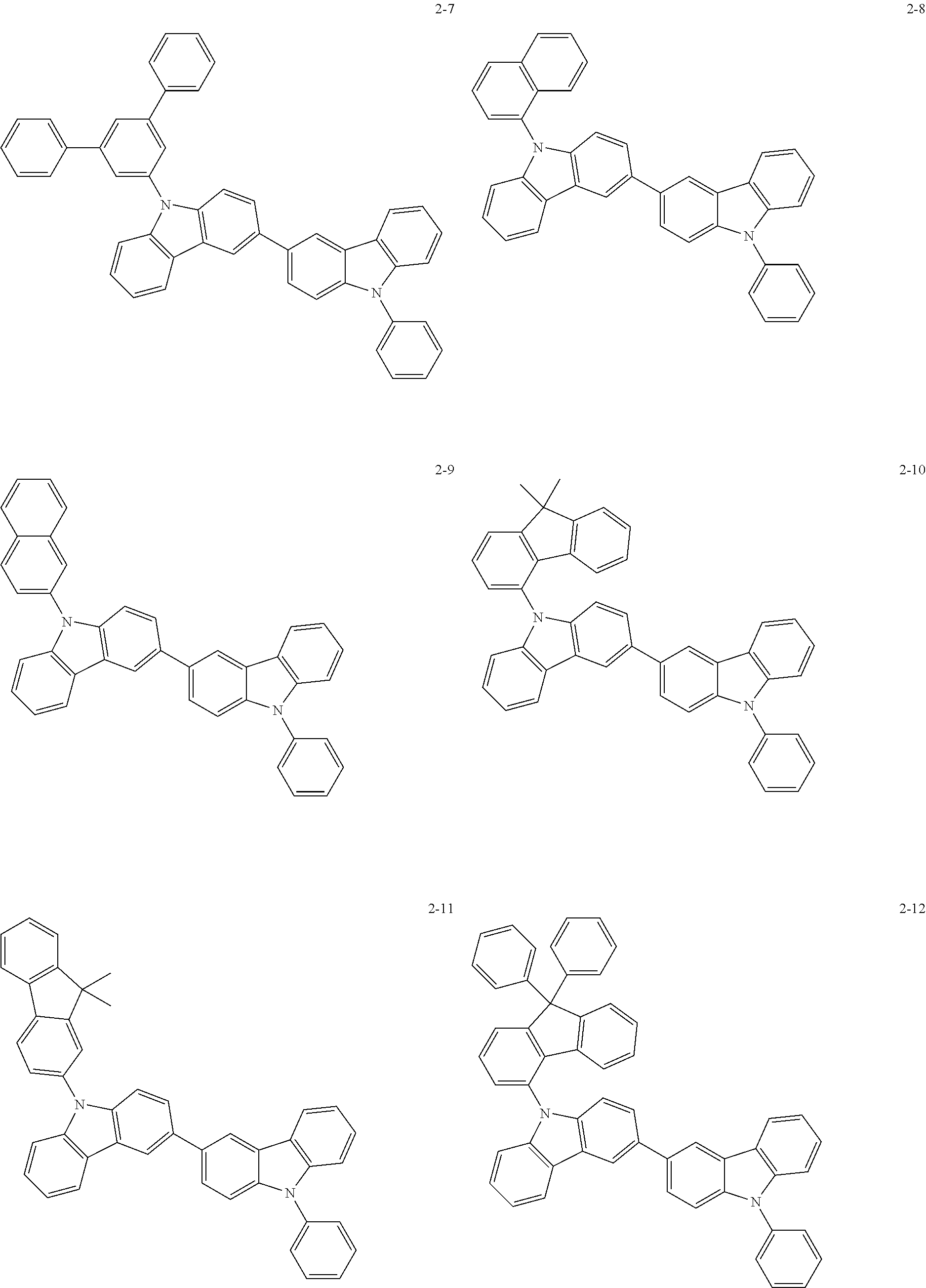

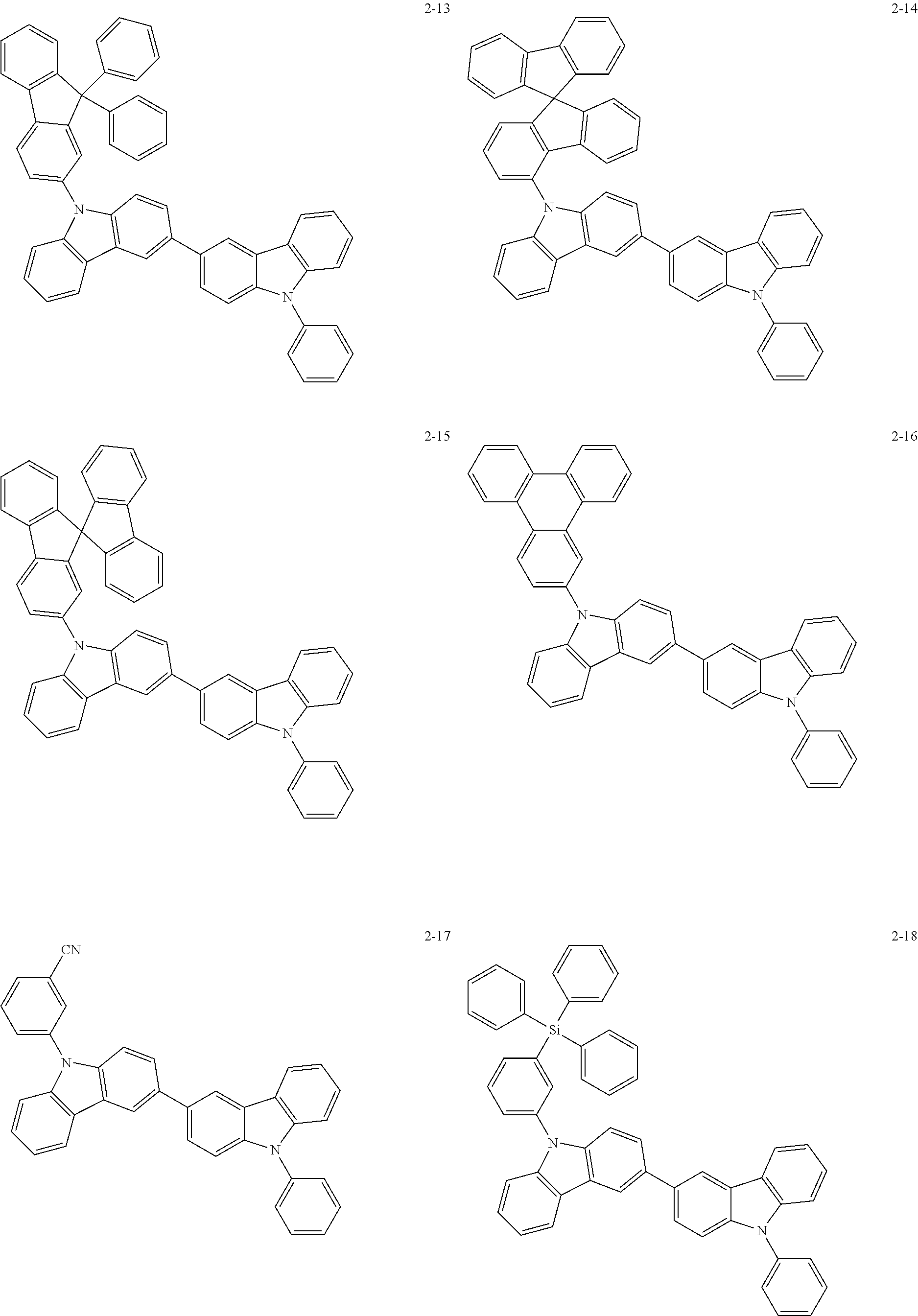

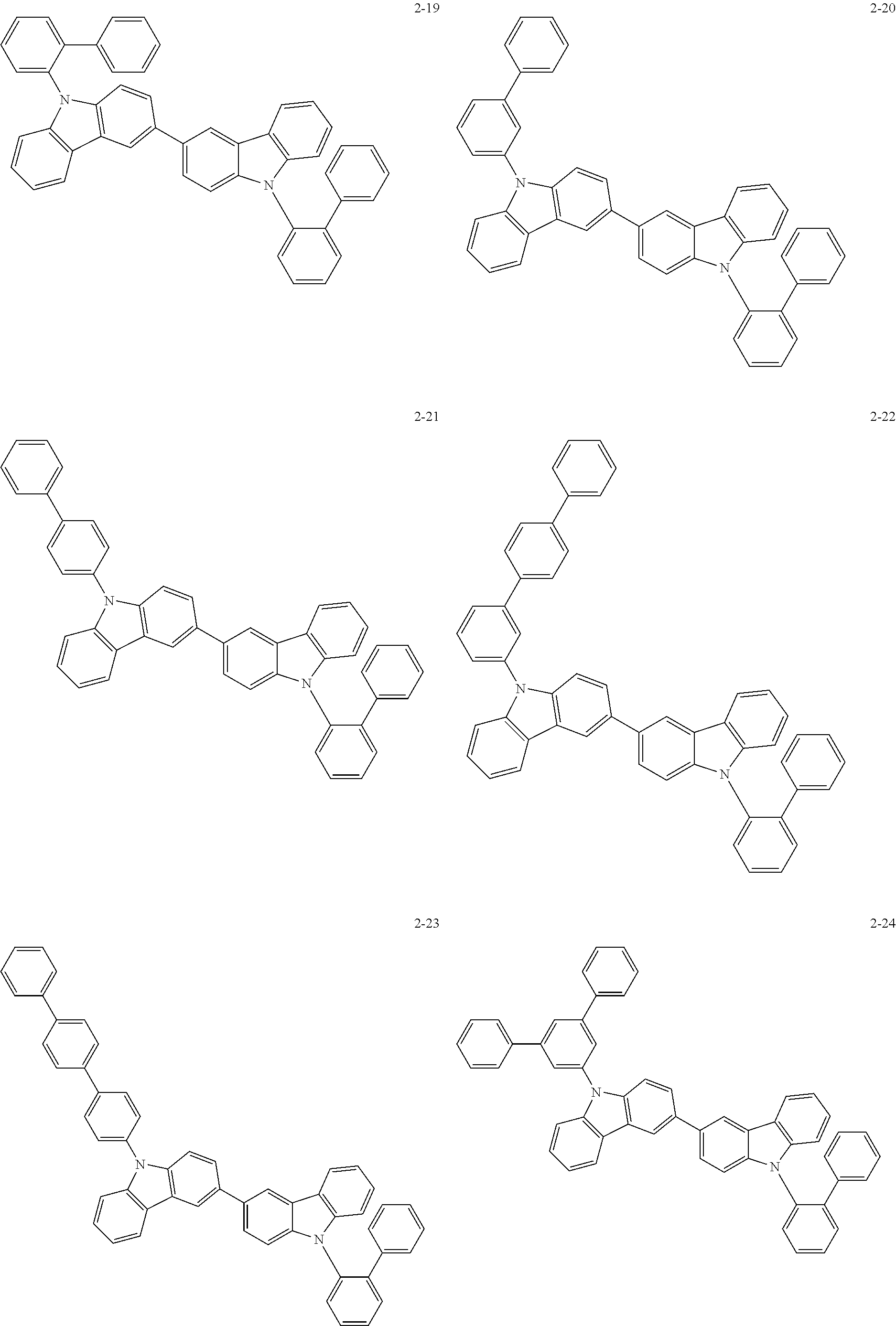

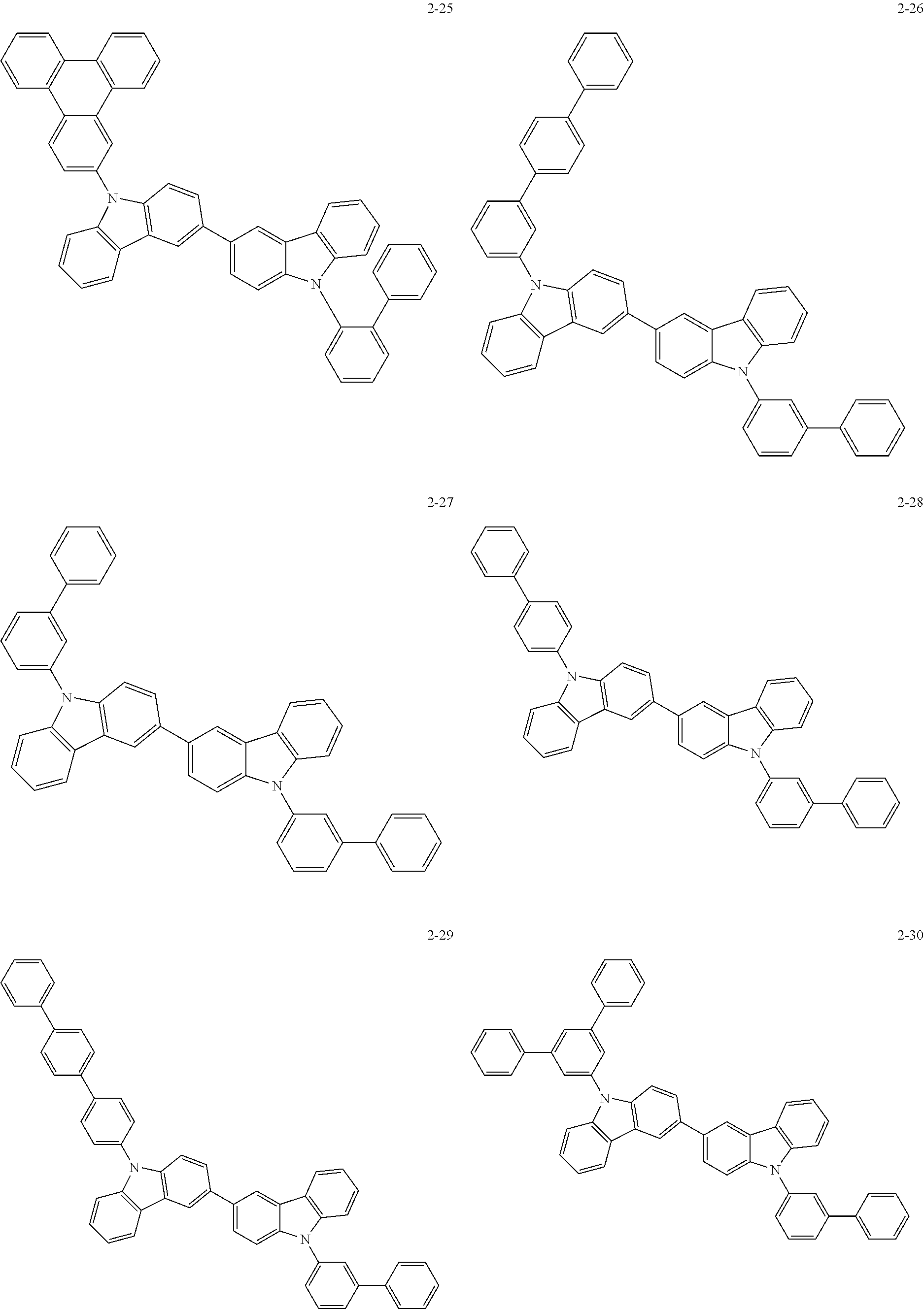

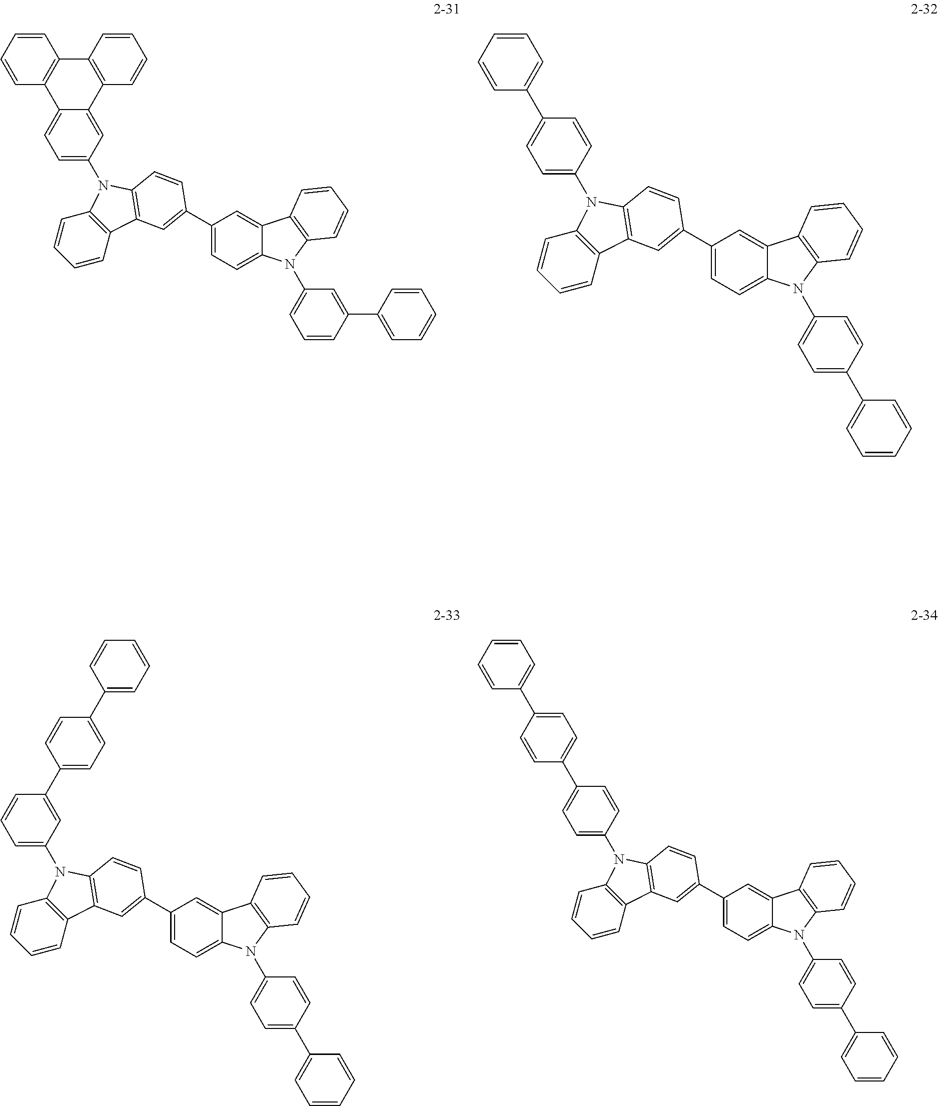 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

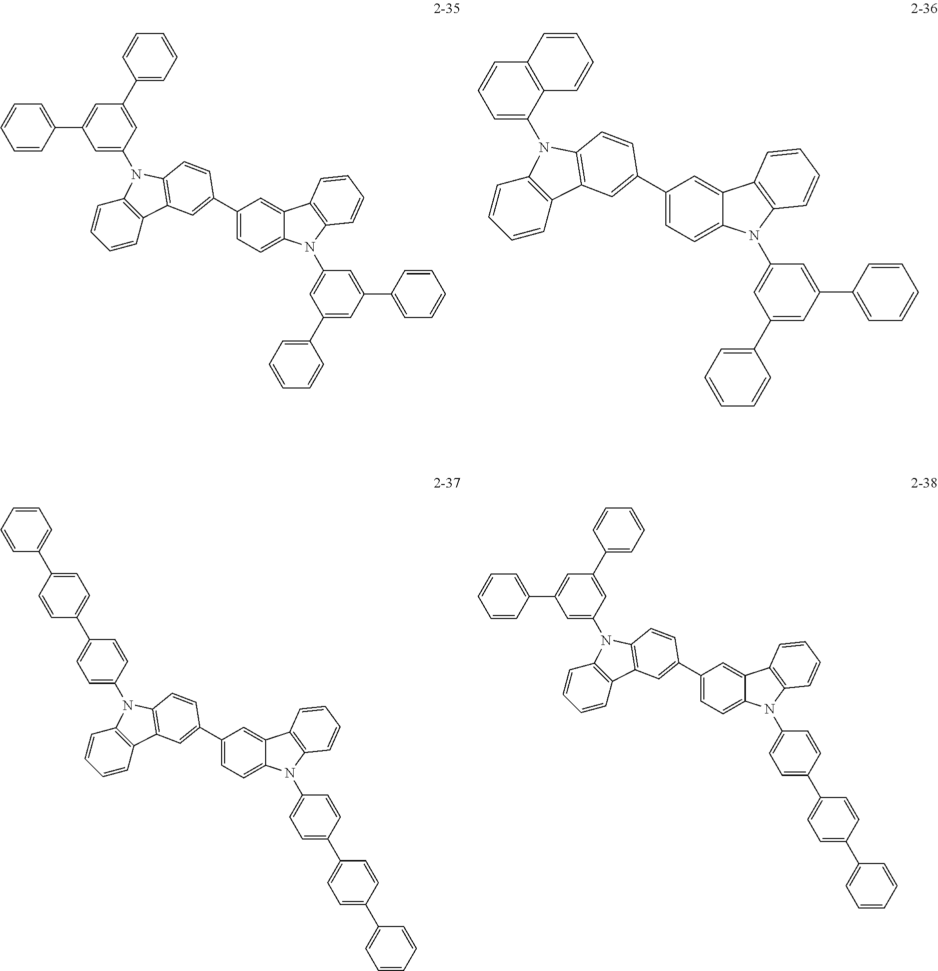 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

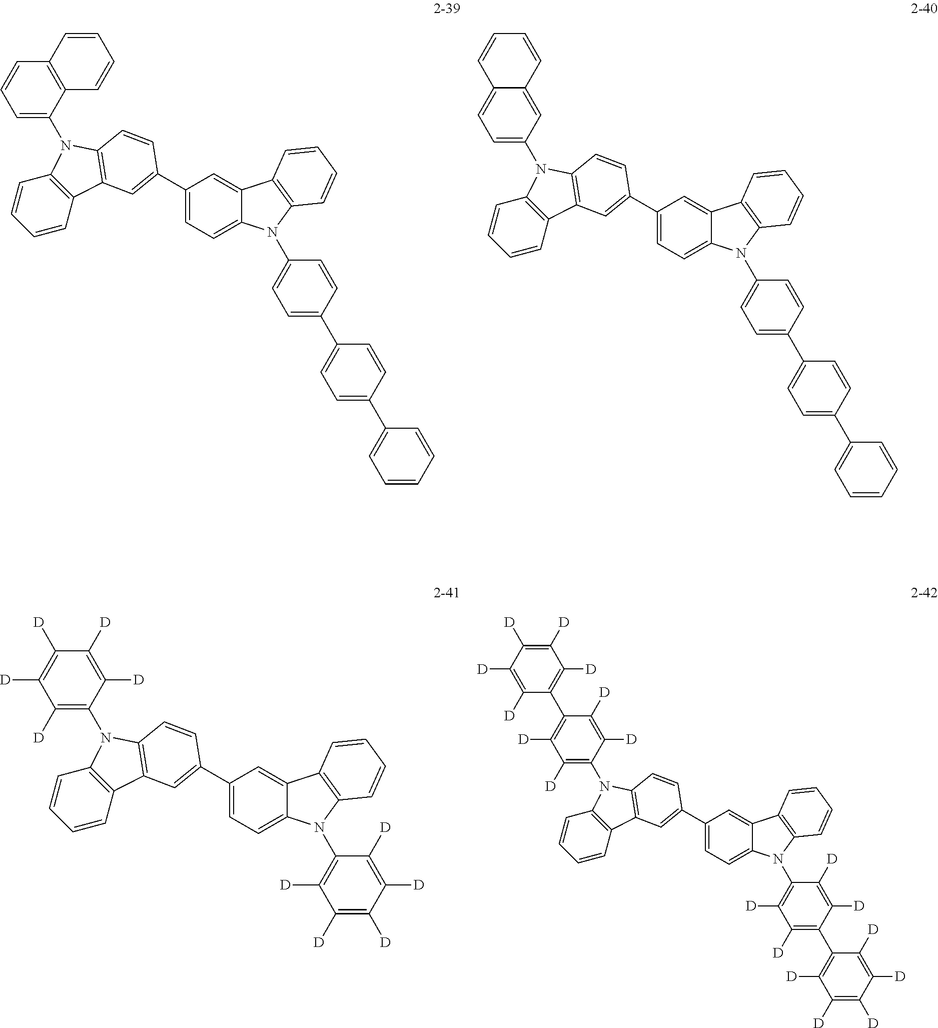 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

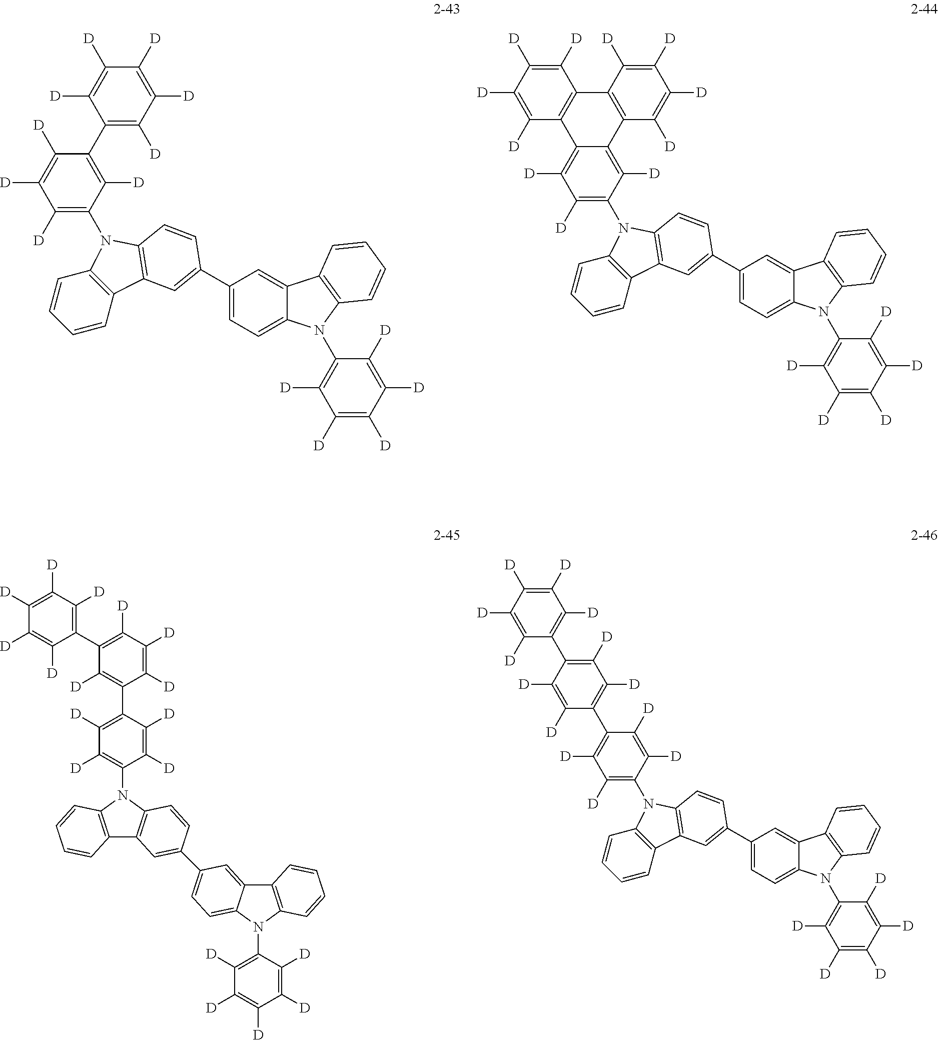

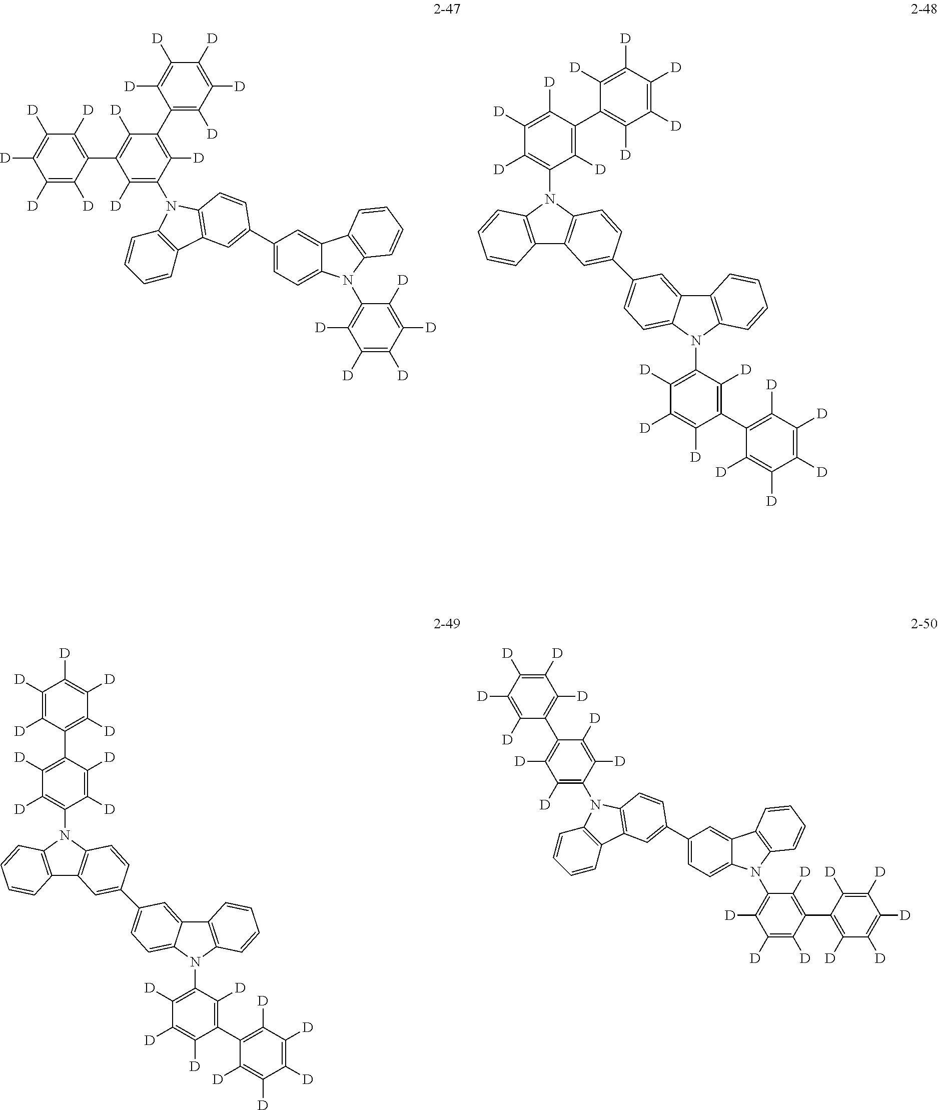

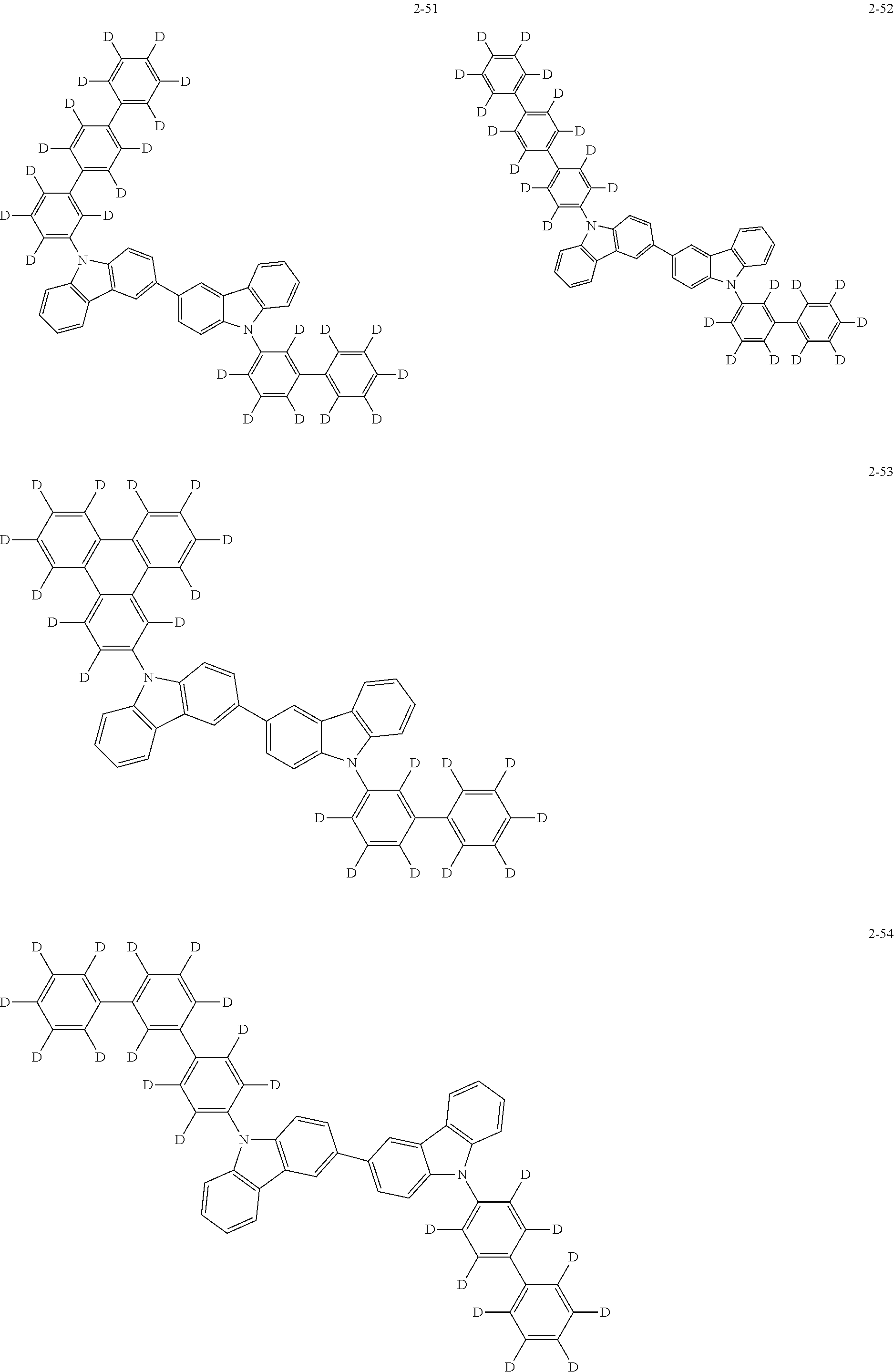

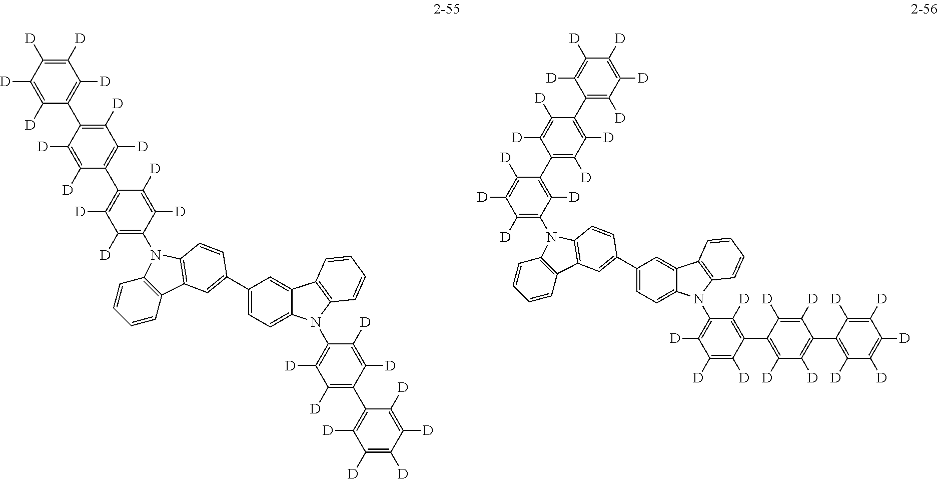 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c3[2H])c([2H])c2[2H])c([2H])c1[2H]]

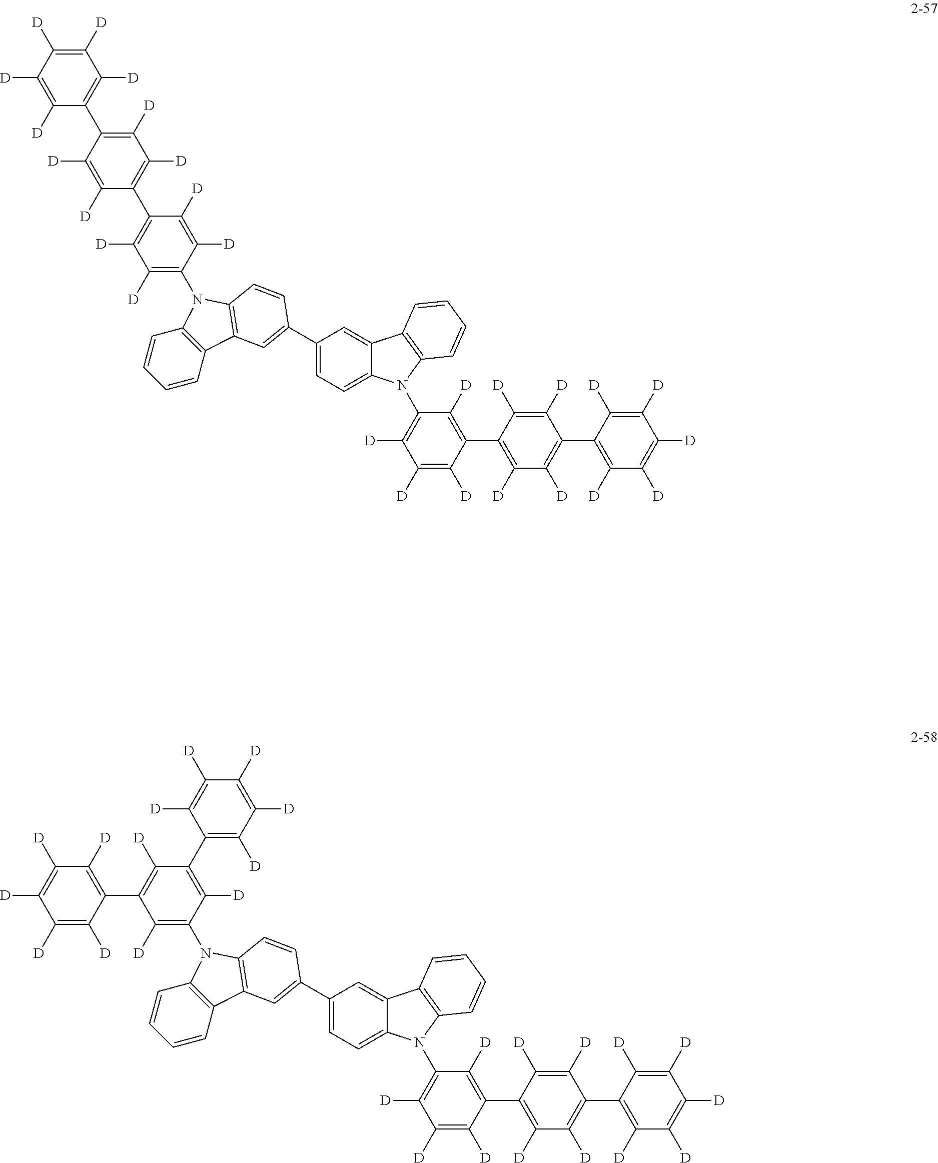

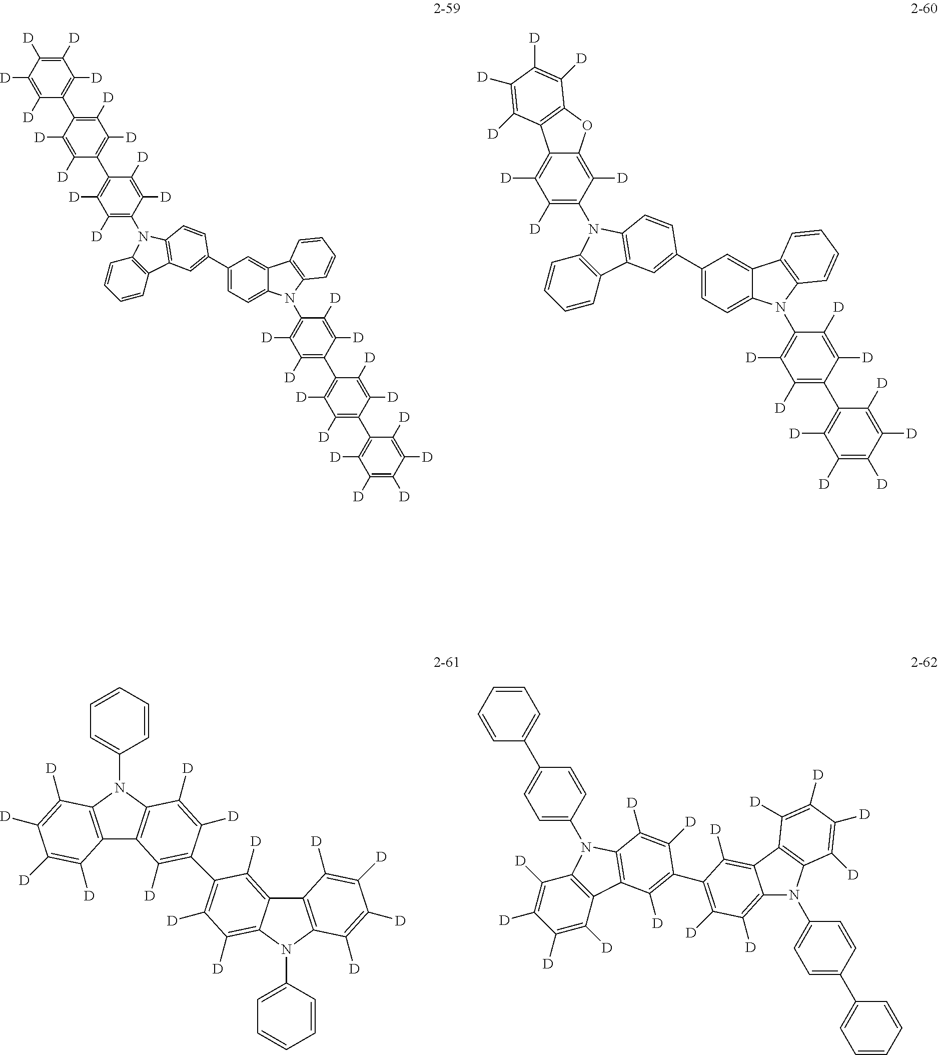

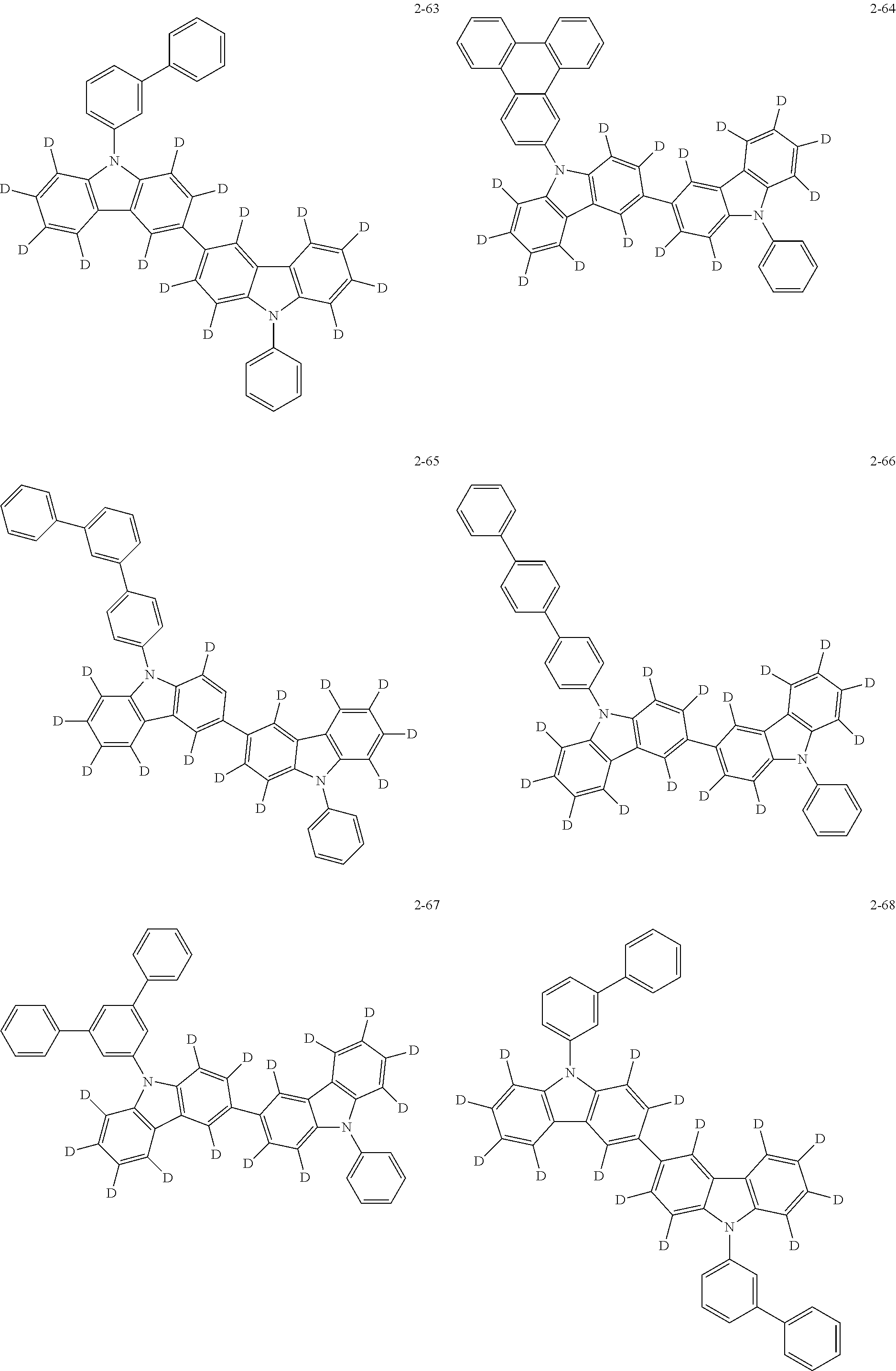

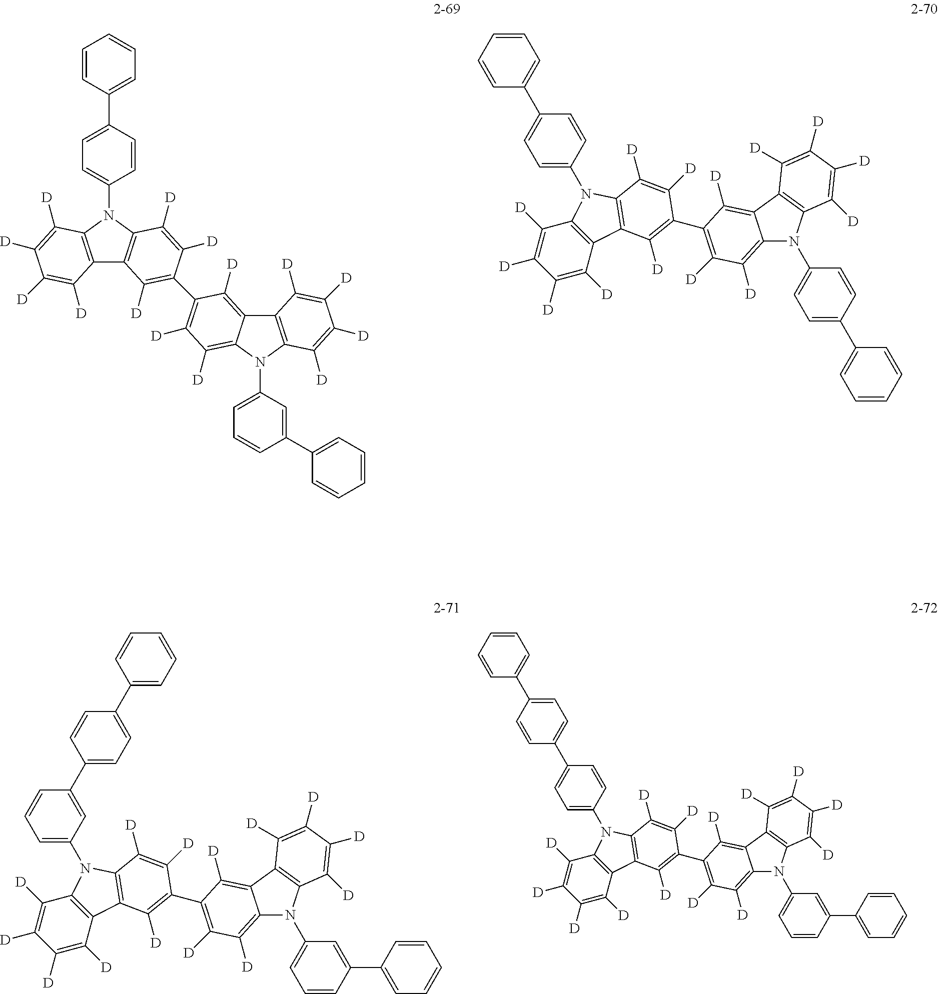

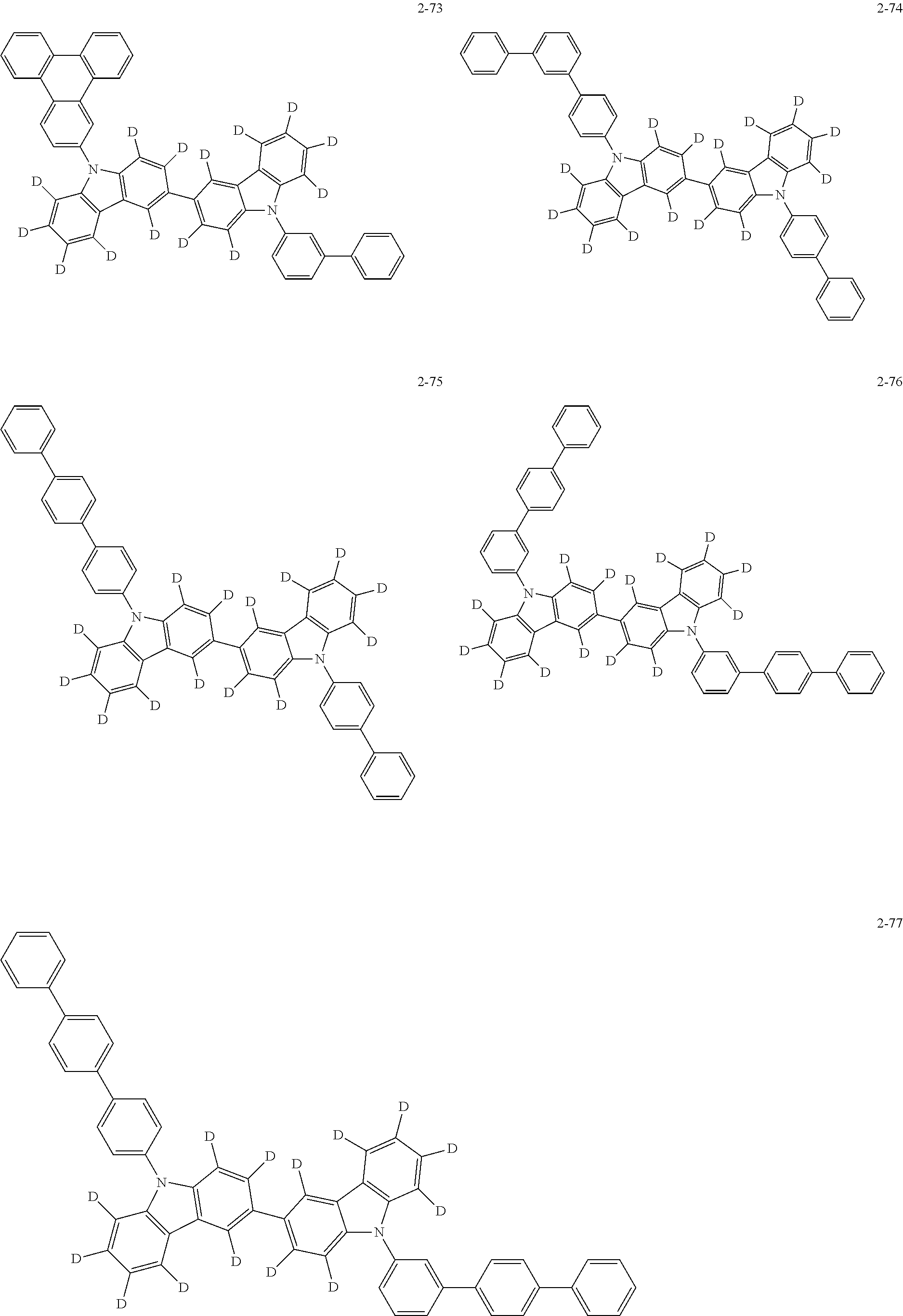

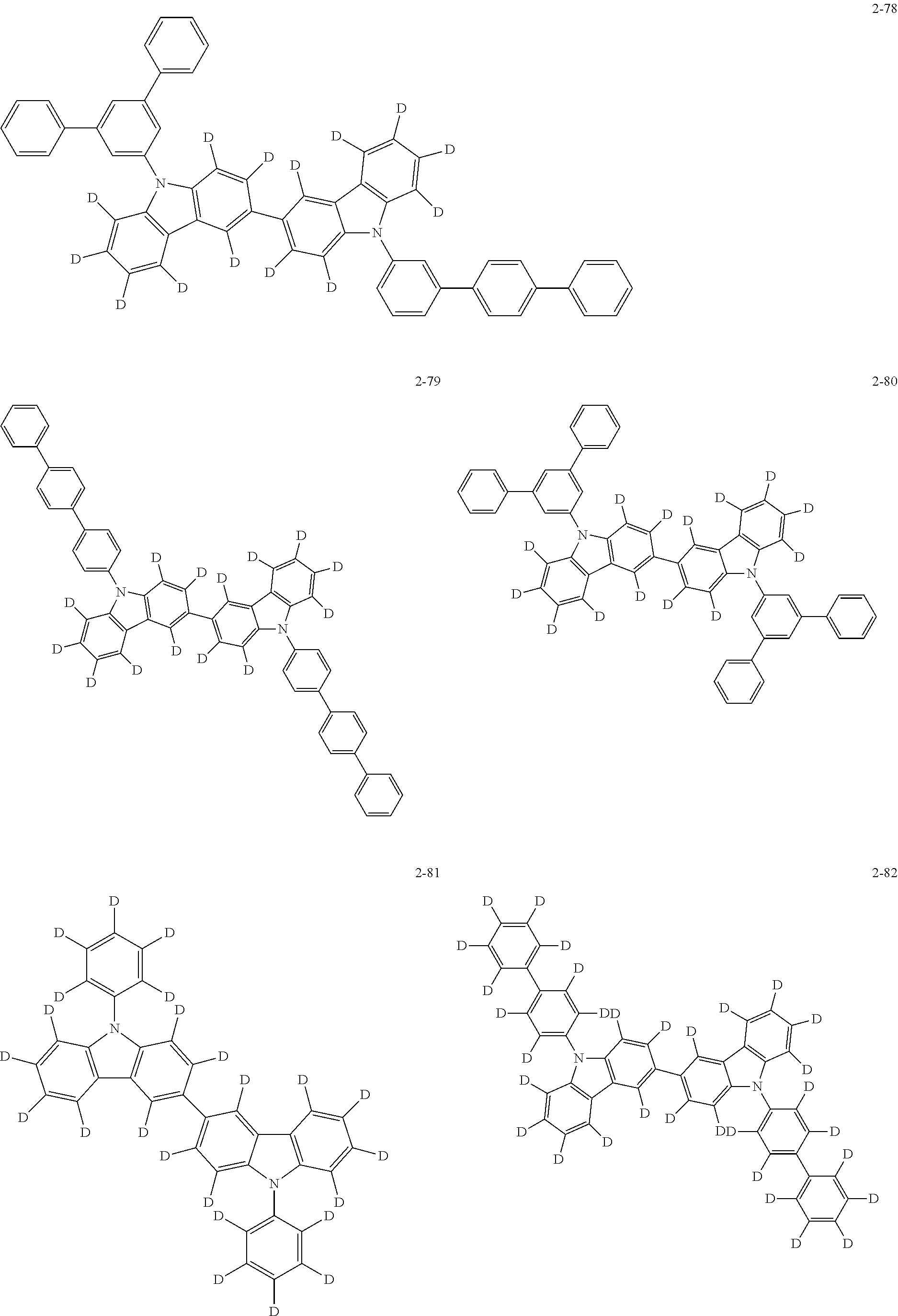

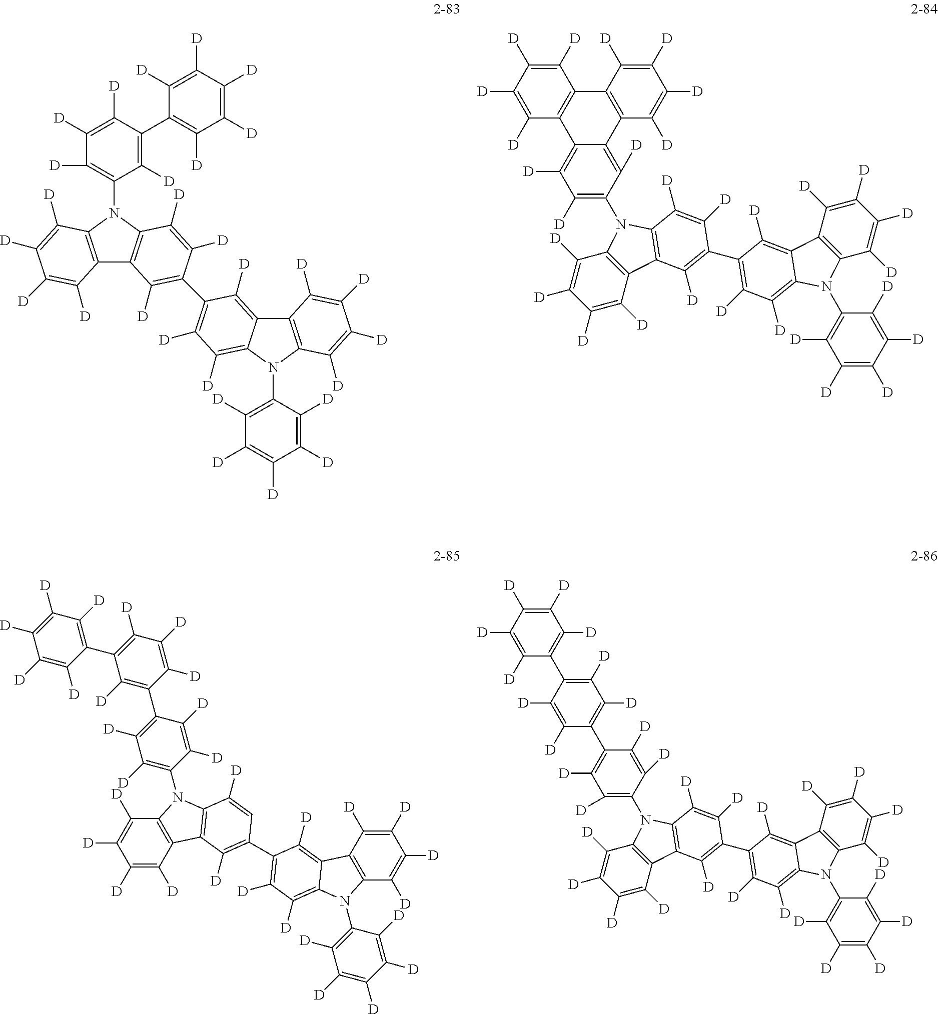

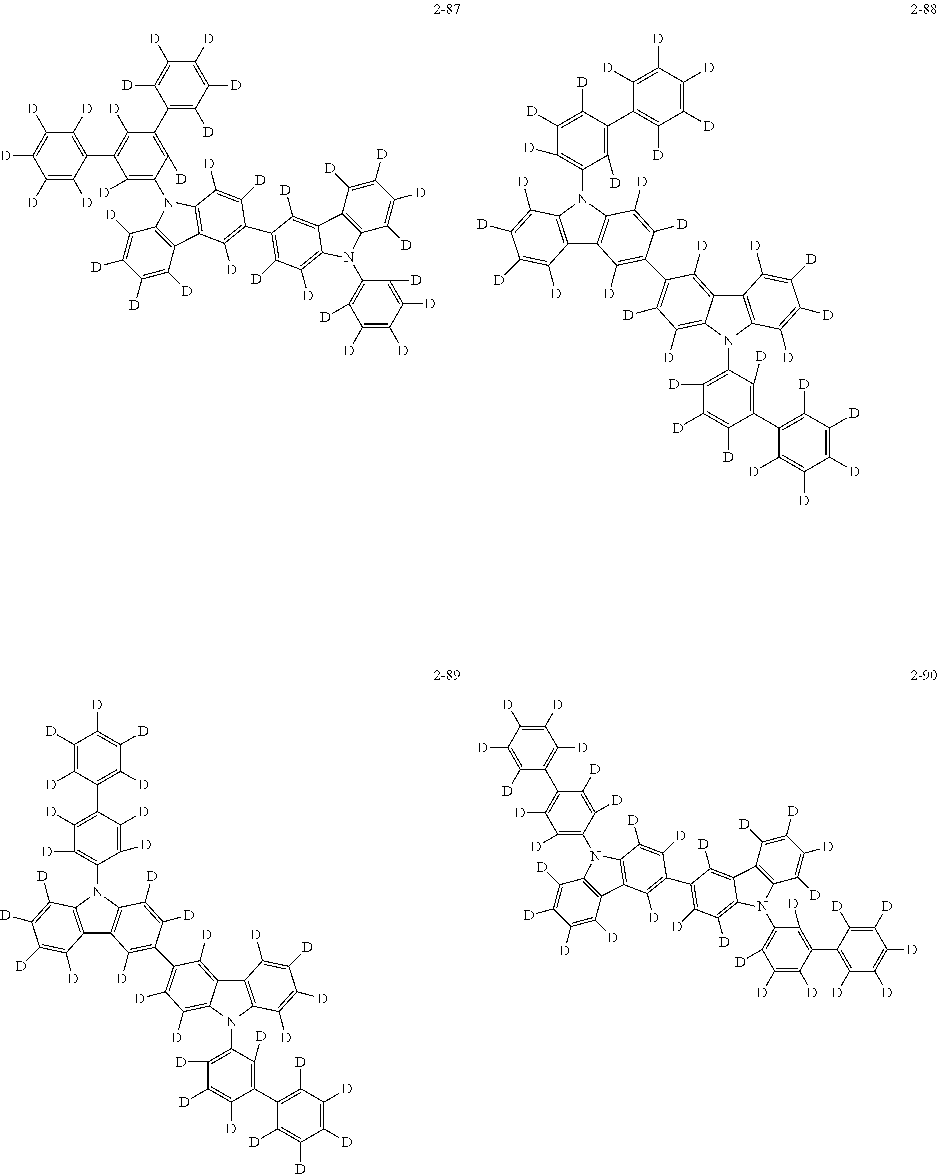

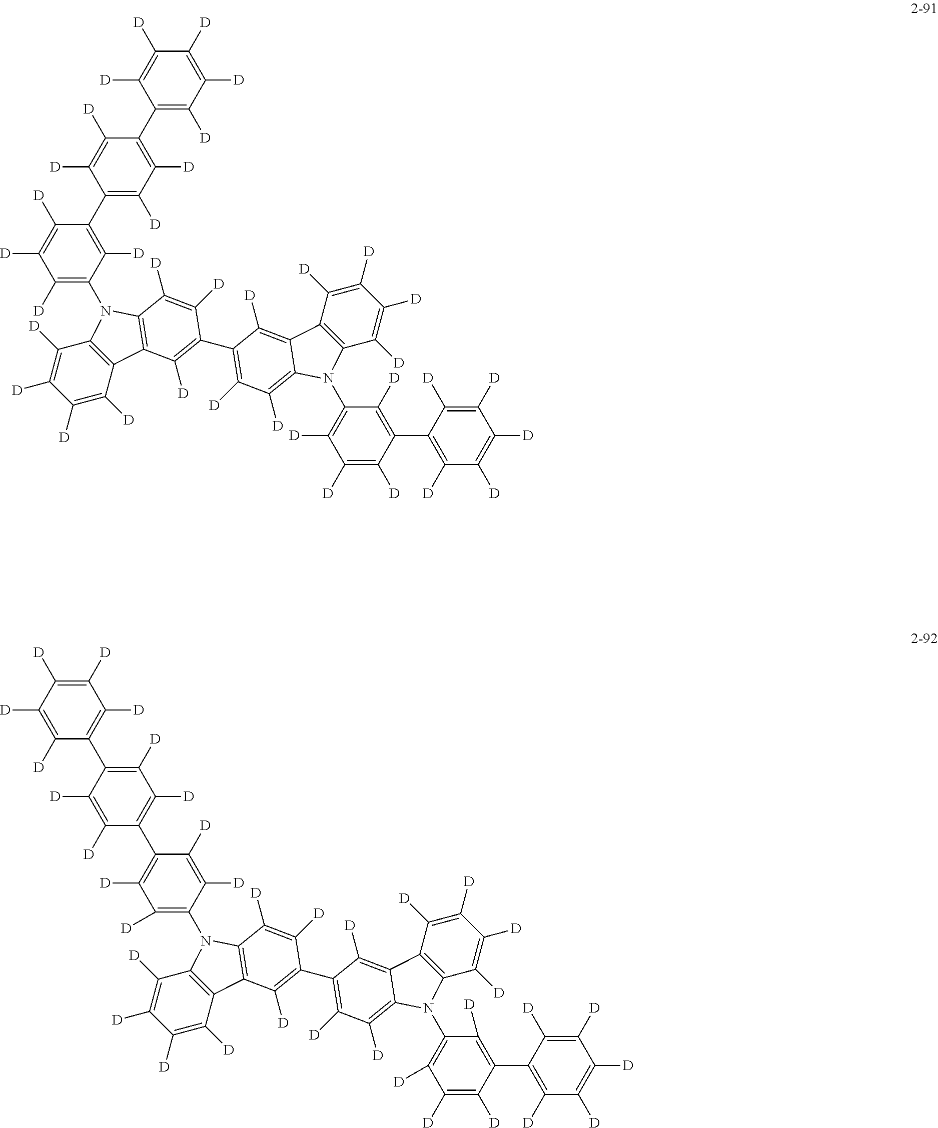

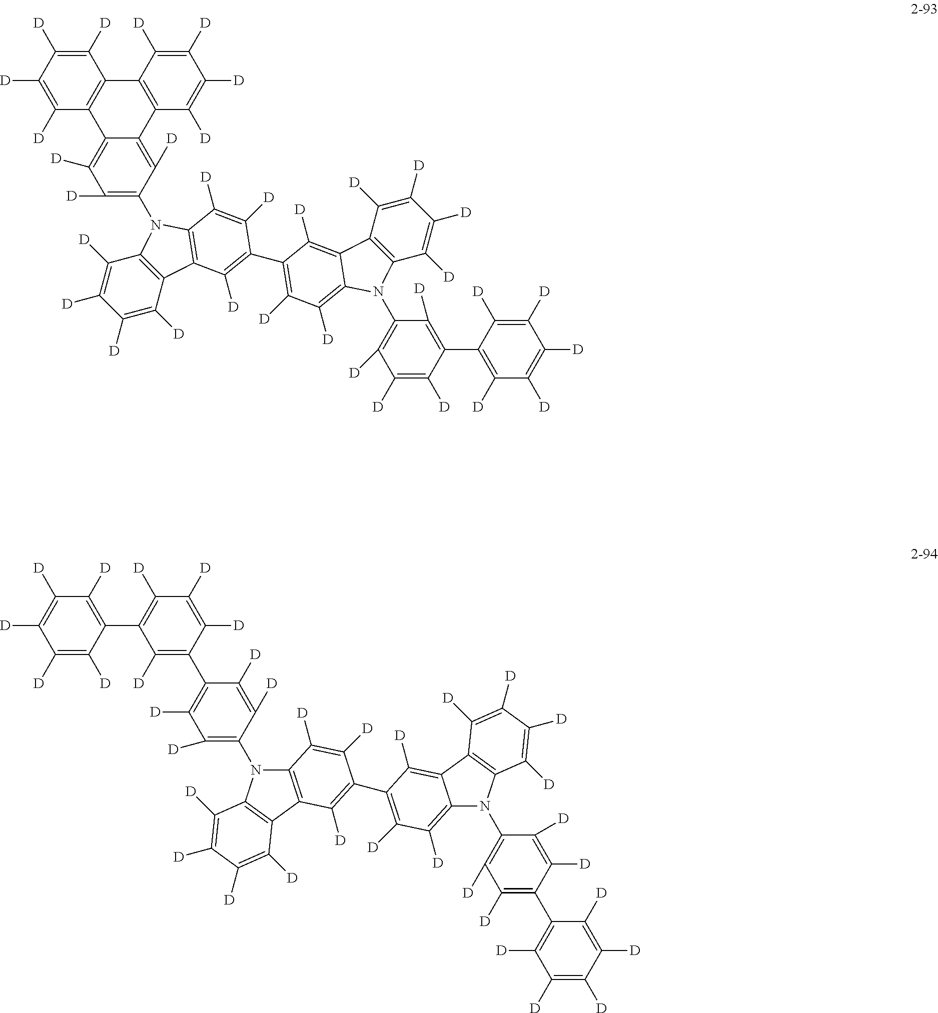

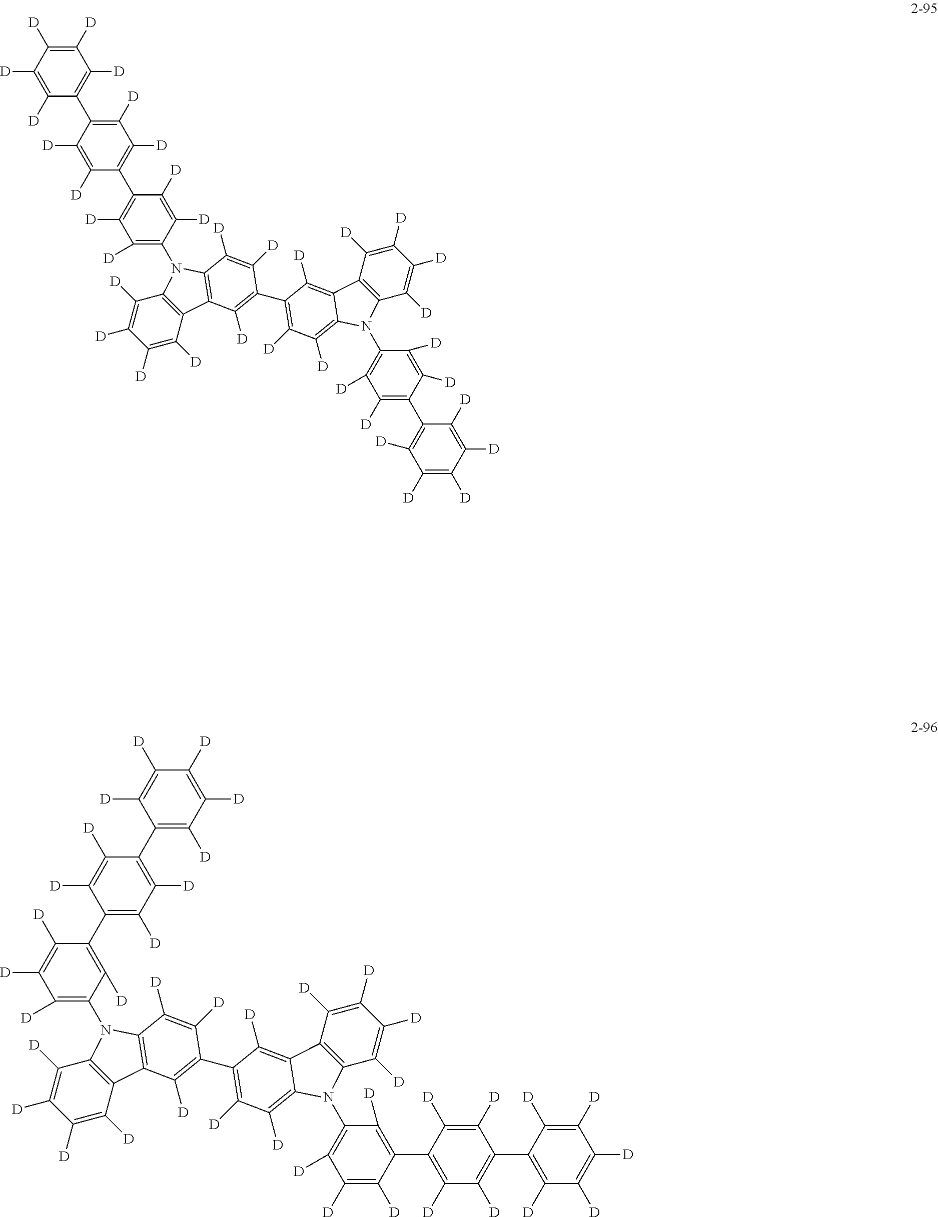

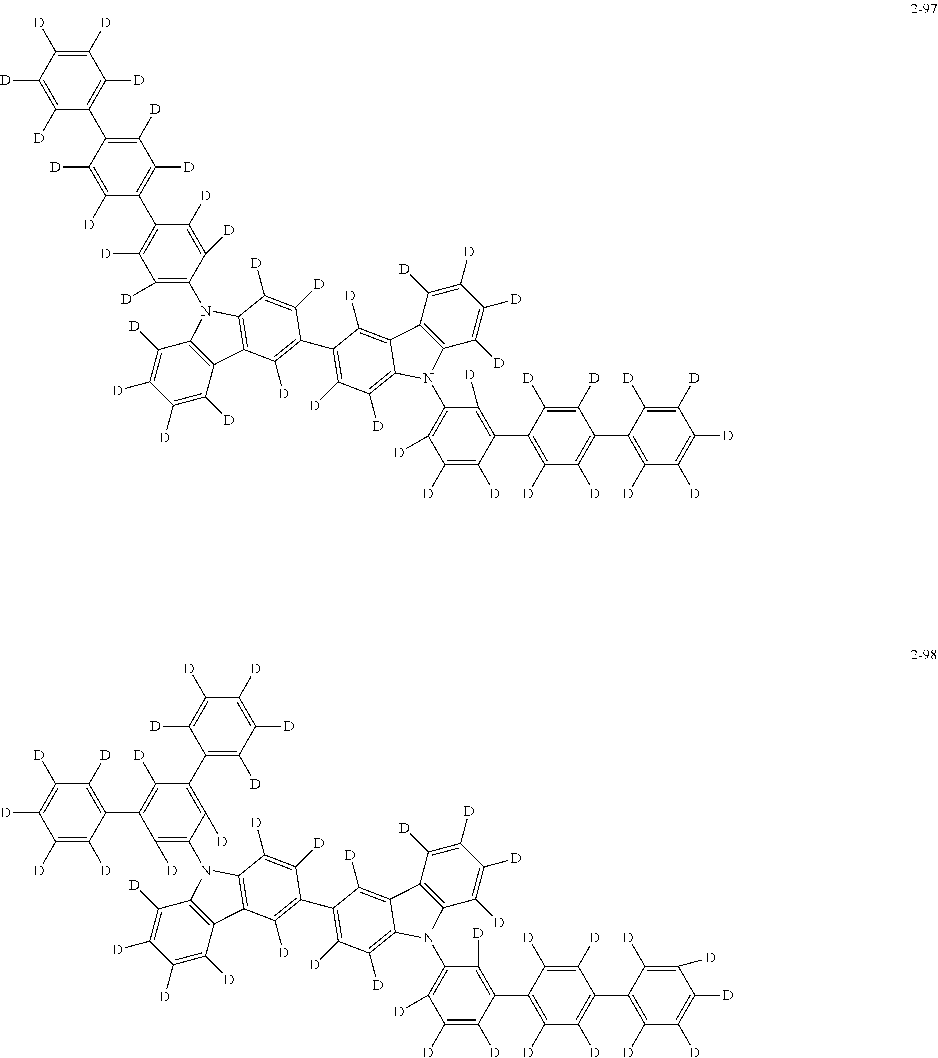

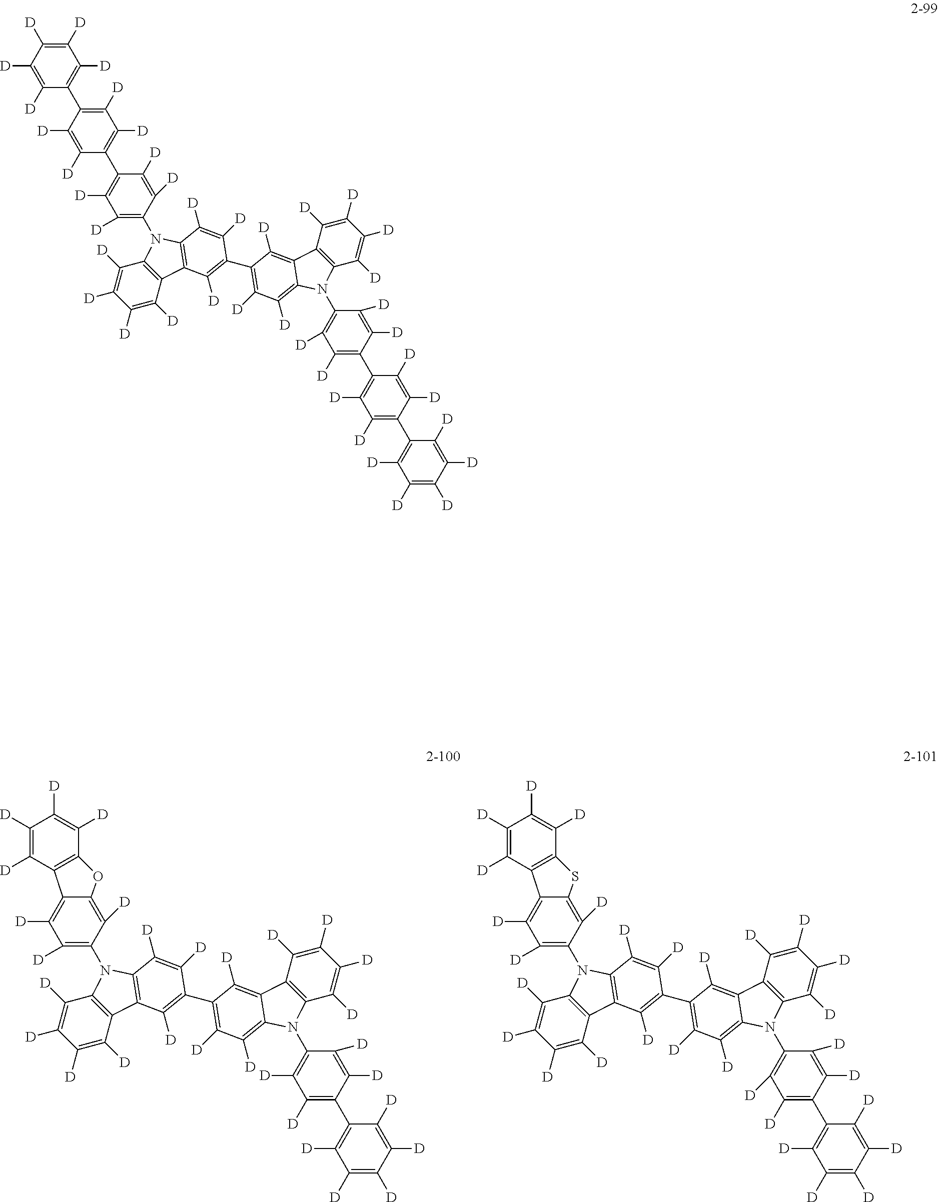

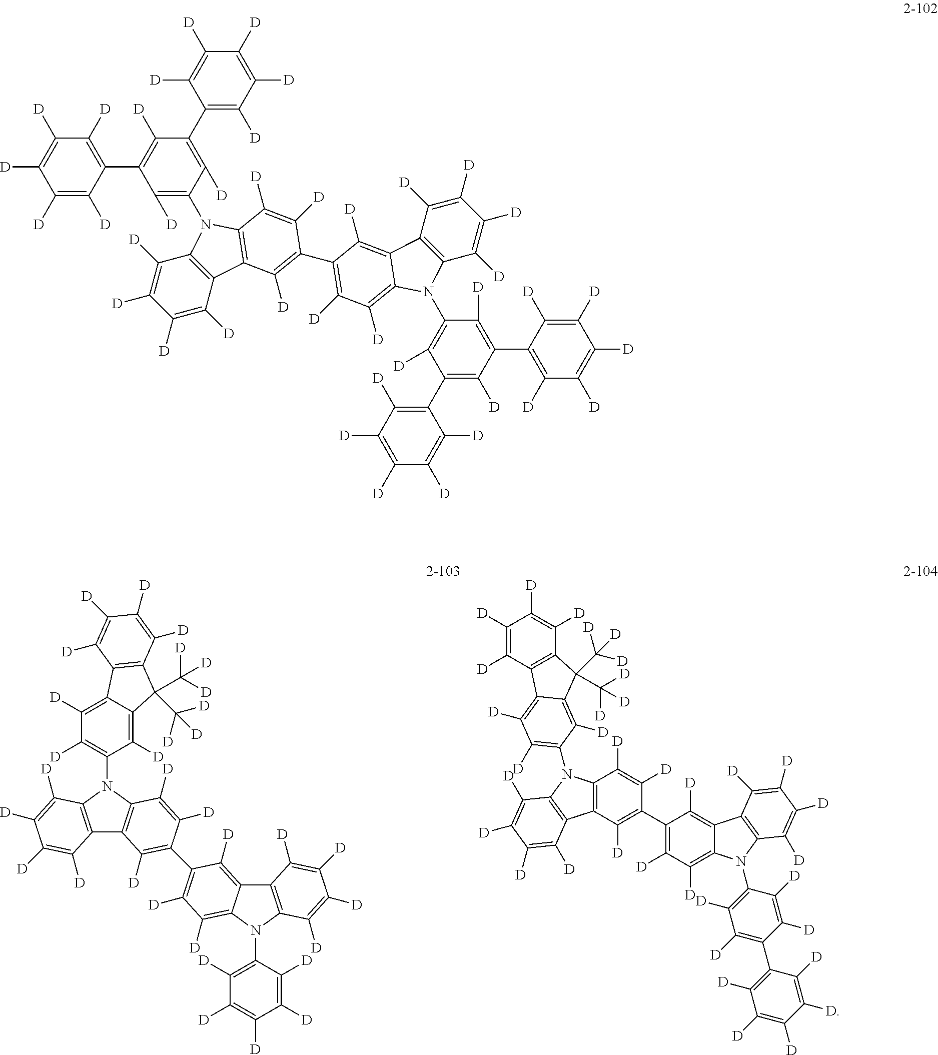

14. The organic light emitting device of claim 10, wherein the organic material layer comprises an emissive layer, and
the emissive layer comprises the heterocyclic compound of Formula 1.

15. The organic light emitting device of claim 11, wherein the organic material layer comprises an emissive layer, and
the emissive layer comprises the heterocyclic compound of Formula 1 and the compound of Formula 2.

16. The organic light emitting device of claim 10, further comprising one or more layers selected from the group consisting of an emissive layer, a hole injection layer, a hole transport layer, an electron injection layer, an electron transport layer, an electron blocking layer, and a hole blocking layer.

17. A composition for an organic material layer of an organic light emitting device, comprising the heterocyclic compound of claim 1.

18. The composition of claim 17, further comprising a compound of Formula 2 below: 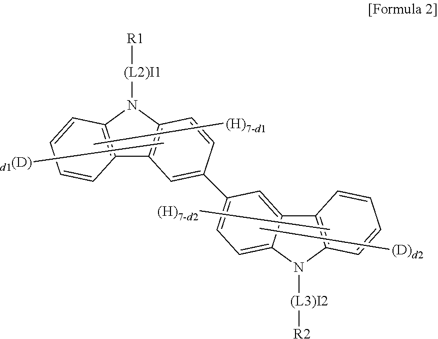
In Formula 2,
L2 and L3 are each independently a direct bond; or a substituted or unsubstituted arylene group having 6 to 60 carbon atoms,
each of I1 and I2 is an integer of 1 to 3, and when it is 2 or higher, each substituent in the parentheses is the same or different,
R1 and R2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group 6 to 60 carbon atoms; or a substituted or unsubstituted heteroaryl group having 2 to 60 carbon atoms,
H is hydrogen, and D is deuterium, and
d1 and d2 are each independently an integer of 0 to 7.

19. The composition of claim 18, wherein the weight ratio of the heterocyclic compound of Formula 1 and the compound of Formula 2 in the composition is 1:10 to 10:1.
